(19) United States
(12) Patent Application Publication
(10) Pub. No.: US 2023/0183743 A1
(43) Pub. Date: Jun. 15, 2023

(54) COMPOSITIONS AND METHODS FOR TREATING GJB2-ASSOCIATED HEARING LOSS

(71) Applicant: Akouos, Inc., Boston, MA (US)

(72) Inventors: Emmanuel John SIMONS, Boston, MA (US); Robert NG, Boston, MA (US); Danielle R. LENZ, Boston, MA (US); Hao CHIANG, Boston, MA (US)

(21) Appl. No.: 17/998,486

(22) PCT Filed: May 13, 2021
(86) PCT No.: PCT/US21/32354

§ 371 Date: Nov. 10, 2022

Related U.S. Application Data
(60) Provisional application No. 63/152,835, filed on Feb. 23, 2021.
(60) Provisional application No. 63/024,468, filed on May 13, 2020.

(51) Int. Cl. C12N 15/86; C07K 14/705; A61P 27/16; A61M 25/00

(52) U.S. Cl. CPC C12N 15/86; C07K 14/705; A61P 27/16; A61M 25/0021; A61M 25/0084; C12N 2750/14143; C12N 2750/14171; C12N 2830/008; C12N 2830/50; A61K 48/00

(57) ABSTRACT

The present disclosure provides constructs comprising a coding sequence operably linked to a promoter, wherein the coding sequence encodes a connexin 26 protein. Exemplary constructs include AAV constructs. Also provided are methods of using disclosed constructs for the treatment of hearing loss and/or deafness.

### CROSS REFERENCE TO RELATED APPLICATIONS

This application claims the benefit of U.S. Provisional Application No. 63,024/468, filed May 13, 2020 and U.S. Provisional Application No. 63/152,835, filed Feb. 23, 2021, which are incorporated herein by reference in their entirety.

### REFERENCE TO SEQUENCE LISTING SUBMITTED ELECTRONICALLY

The content of the electronically submitted sequence listing in ASCII text file (Name: 4833_006PC02_Seqlisting_ST25.txt; Size: 227,027 bytes; and Date of Creation: May 13, 2021) filed with the application is incorporated herein by reference in its entirety.

### BACKGROUND

Hearing loss can be conductive (arising from the ear canal or middle ear), sensorineural (arising from the inner ear or auditory nerve), or mixed. Most forms of nonsyndromic deafness are associated with permanent hearing loss caused by damage to structures in the inner ear (sensorineural deafness), although some forms may involve changes in the middle ear (conductive hearing loss). The great majority of human sensorineural hearing loss is caused by abnormalities in the hair cells of the organ of Corti in the cochlea (poor hair cell function). The hair cells may be abnormal at birth, or may be damaged during the lifetime of an individual (e.g., as a result of noise trauma or infection).

### SUMMARY

The present disclosure provides the recognition that diseases or conditions associated with hearing loss can be treated via, e.g., the replacement or addition of certain gene products. The present disclosure further provides that gene products involved in the development, function, and/or maintenance of inner ear cells can be useful for treatment of diseases or conditions associated with hair cell and/or supporting cell loss. The present disclosure thus provides for the administration of compositions that result in expression of gene products involved in the development, function, and/or maintenance of inner ear cells including supporting cells and hair cell, and/or the use of such compositions in the treatment of hearing loss, or diseases or conditions associated with hearing loss. In some embodiments, a gene product can be encoded by a gap junction beta-2 (GJB2) gene (the GJB2 gene encodes connexin 26 protein) or a characteristic portion thereof. In some embodiments, a gene product can be connexin 26 protein (encoded by a GJB2 gene) or a characteristic portion thereof.

The present disclosure further provides that AAV particles can be useful for administration of compositions that result in expression of gene products involved in the development, function, and/or maintenance of inner ear cells, and/or the treatment of hearing loss, or diseases or conditions associated with hearing loss. As described herein, AAV particles comprise (i) a AAV polynucleotide construct (e.g., a recombinant AAV (rAAV) polynucleotide construct), and (ii) a capsid comprising capsid proteins. In some embodiments, an AAV polynucleotide construct comprises a GJB2 gene or a characteristic portion thereof.

The present disclosure further provides compositions comprising polynucleotide constructs comprising a GJB2 gene or a characteristic portion thereof. In some embodiments, a construct may further include regulatory elements operably attached to a coding sequence. In certain embodiments, included regulatory elements facilitate tissue specific expression at physiologically suitable levels.

Also provided herein are methods of administering constructs and compositions described herein. In certain embodiments, administration involves surgical intervention and the delivery of rAAV particles comprising therapeutic constructs. In certain embodiments AAV particles may be delivered to the inner ear of a subject in need thereof by surgical introduction through the round window membrane. In some embodiments, efficacy of an intervention is determined through established tests, and measurements are compared to known control measurements.

### BRIEF DESCRIPTION OF THE DRAWING

FIG. 1 panel (A) depicts a simplified endogenous AAV genome; panel (B) depicts a simplified recombinant AAV (rAAV) construct capable of expressing a GJB2 gene.

FIG. 2A-2O show panels (A)-(O), which depict alternative exemplary rAAV constructs comprising a GJB2 gene. FIG. 2A depicts a rAAV construct comprising a 5′ ITR, a CAG promoter, a nucleic acid encoding a hGJB2 gene, a bGH polyA, and a 3′ ITR. FIG. 2B depicts a rAAV construct comprising a 5′ ITR, a CAG promoter, a nucleic acid encoding a hGJB2 gene, a 3′ UTR, a bGH polyA, and a 3′ ITR. FIG. 2C depicts a rAAV construct comprising a 5′ ITR, a GJB2 promoter, a nucleic acid encoding a hGJB2 gene, a bGH polyA, a C3 domain, and a 5′ ITR. FIG. 2D depicts a rAAV construct comprising a 5′ ITR, a GJB2 promoter, a nucleic acid encoding a hGJB2 gene, a bGH polyA, a D7 domain, and a 3′ ITR. FIG. 2E depicts a rAAV construct comprising a 5′ ITR, a GJB2 promoter, a hGJB2 gene, a bGH polyA, and a 3′ ITR. FIG. 2F depicts a rAAV construct comprising a 5′ ITR, a CAG promoter, a 5′ UTR, a hGJB2 gene, a FLAG tag, a 3′ UTR, a bGH polyA, and a 3′ ITR. FIG. 2G depicts a rAAV construct comprising a 5′ ITR, a smCBA promoter, a 5′ UTR, a nucleic acid encoding a hGJB2 gene, a FLAG tag, a 3′ UTR, a bGH polyA, and a 3′ ITR. FIG. 2H depicts a rAAV construct comprising a 5′ ITR, a promoter comprising a CMV promoter and a hGJB2 promoter, a 5′ UTR, a nucleic acid encoding a hGJB2 gene, a FLAG tag, a 3′ UTR, a bGH polyA, and a 3′ ITR. FIG. 2I depicts a rAAV construct comprising a 5′ ITR, a promoter comprising a CMV promoter and a GFAP promoter, a 5′ UTR, a nucleic acid encoding a hGJB2 gene, a FLAG tag, a 3′ UTR, a bGH polyA, and a 3′ ITR. FIG. 2J depicts a rAAV construct comprising a 5′ ITR, a GFAP inner ear supporting cell-specific promoter, a 5′ UTR, a nucleic acid encoding a hGJB2 gene, a FLAG tag, a 3′ UTR, a bGH polyA, and a 3′ ITR. FIG. 2K depicts a rAAV construct comprising a 5′ ITR, a CAG promoter, a 5′ UTR, a nucleic acid encoding a hGJB2 gene, a FLAG tag, a destabilization domain, a 3′ UTR, a bGH polyA, and a 3′ ITR. FIG. 2L depicts a rAAV construct comprising a 5′ ITR, a promoter comprising a hGJB2 enhancer and a hGJB2 promoter, a 5′ UTR, a nucleic acid encoding a hGJB2 gene, a FLAG tag, a 3′ UTR, a bGH polyA, and a 3′ ITR. FIG. 2M depicts a rAAV construct comprising a 5′ ITR, a CAG promoter, a 5′ UTR, a hGJB2 promoter, a hGJB2 gene, a FLAG tag, a microRNA regulatory target site, a 3′ UTR, a bGH polyA, and a 3′ ITR. FIG. 2N depicts a rAAV construct comprising a 5′ ITR, a promoter comprising an inner ear supporting cell specific promoter and a hGJB2 minimal promoter, a nucleic acid encoding a hGJB2 gene, a FLAG tag, a 5′ UTR, a bGH polyA, and a 3′ ITR. FIG. 2O depicts a rAAV construct comprising a 5′ ITR, a CAG promoter, a nucleic acid encoding a hGJB2 gene, a FLAG tag, a T2A element, a nucleic acid encoding eGFP, a bGH polyA, and a 3′ ITR

FIG. 3 depicts connexin 26 (Cx26)/GJB2 protein expression from HEK293FT cells that have been exposed to exemplary constructs described herein. Panel (A) Depicts Cx26 protein expression in HEK293FT cells that have been transfected with exemplary rAAV constructs comprising CAG promoters, bands corresponding to Vinculin and Cx26 are marked. Panel (B) Depicts GJB2 protein expression in HEK293FT cells that have been transfected with exemplary constructs comprising hGJB2 coding sequences with GJB2 5′ UTR and 3′UTR sequences, driven by CAG, CMVe-GJB2p, or smCBA promoter/enhancer sequences as noted, bands corresponding to GAPDH and GJB2-FLAG are marked. Panel (C) Depicts GJB2 protein expression in HEK293FT cells that have been transduced with exemplary rAAV particles comprising constructs comprising hGJB2 coding sequences with GJB2 5′ UTR and 3′UTR sequences, driven by CAG, CMVe-GJB2p, or smCBA promoter/enhancer sequences as noted, bands corresponding to GAPDH and GJB2-FLAG are marked, positive control is hGJB2 coding sequence driven by CAG promoter/enhancer without GJB2 5′ UTR or 3′ UTR.

FIG. 4 depicts quantitative PCR (qPCR) results of GJB2 mRNA expression in HEK293FT cells and wild type neonatal CD1 explants that have been transduced with exemplary rAAV constructs.

FIG. 5, panels (A) and (B) depict eGFP protein expression in HEK293T cells under the power of various exemplary promoters, cells were sorted and quantified 72 hours after transfection.

FIG. 6 depicts FLAG protein expression in mouse cochlear explants transduced at P2 with exemplary rAAVAnc80 particles comprising constructs driven by CAG, CMVe-GJB2p, or smCBA promoter/enhancer sequences as noted, explants were fixed after 72h, immunostaining for FLAG is noted in green, immunostaining for hair cell marker Myo7a is noted in red, and nuclear marker DAPI is noted in blue. Panel (A) depicts exemplary explants transduced with AAVAnc80-CAG.5UTR.hGJB2.3F.3UTR (SEQ ID NO: 82) at 5.8E9 vg/explant. Panel (B) depicts exemplary explants transduced with AAVAnc80-smCBA.5UTR.hGJB2.3F.3UTR (SEQ ID NO: 83) at 1.4E10 vg/explant. Panel (C) depicts exemplary explants transduced with AAVAnc80-CMVeGJB2p.5UTR.hGJB2.3F.3UTR (SEQ ID NO: 84) at 1.8E10 vg/explant.

FIG. 7 depicts in vitro expression of GJB2 protein in HEK293FT cells transfected with CAG.5UTR.hGJB2.FLAG.miRTS.3UTR (SEQ ID NO: 87), CAG.5UTR.hGJB2.FLAG.3UTR (SEQ ID NO: 82), or CAG.5UTR.hGJB2.FLAG.GFP constructs. CAG.5UTR.hGJB2.FLAG.miRTS.3UTR comprises miRNA targeting sites (miRTS) for miR-182 and miR-183 in the 3UTR to permit exogenous hGJB2 knockdown in the presence of regulatory miR-182 and/or miR-183. To confirm miRNA regulation of constructs, HEK293FT cells were transfected with hGJB2 comprising plasmids and optionally co-transfected with (+) or without (−) plasmids expressing miR-182 and miR-183. 72 h post transfection the cells were harvested for protein and RNA analysis. Panel (A) Depicts exemplary GJB2 protein levels analyzed using western blot; panel (B) depicts exemplary GJB2 mRNA levels analyzed using qPCR.

FIG. 8 illustrates a perspective of a device for delivering fluid to an inner ear, according to aspects of the present disclosure.

FIG. 9 illustrates a sideview of a bent needle sub-assembly, according to aspects of the present disclosure.

FIG. 10 illustrates a perspective view of a device for delivering fluid to an inner ear, according to aspects of the present disclosure.

FIG. 11 illustrates a perspective view of a bent needle sub-assembly coupled to the distal end of a device, according to aspects of the present disclosure.

FIG. 12 depicts in vivo expression of connexin 26 in wild-type mice (p20) that were administered rAAVAnc80 particles comprising CAG.hGJB2.F.GFP (schematic provided in FIG. 2O) to the cochlea. Expression of connexin 26 in the supporting cells and inner hair cells was detected 10 days after administration. Immunostaining of actin filaments and hair cell stereocilia bundles by phalloidin is noted in blue, GFP is noted in green, FLAG is noted in purple, and endogenous connexin 26 is noted in red. SC—supporting cells; IHC—inner hair cells; OHC—outer hair cells.

### DEFINITIONS

The scope of the present disclosure is defined by the claims appended hereto and is not limited by certain embodiments described herein. Those skilled in the art, reading the present specification, will be aware of various modifications that may be equivalent to such described embodiments, or otherwise within the scope of the claims. In general, terms used herein are in accordance with their understood meaning in the art, unless clearly indicated otherwise. Explicit definitions of certain terms are provided below; meanings of these and other terms in particular instances throughout this specification will be clear to those skilled in the art from context.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

The articles “a” and “an,” as used herein, should be understood to include the plural referents unless clearly indicated to the contrary. Claims or descriptions that include “or” between one or more members of a group are considered satisfied if one, more than one, or all of the group members are present in, employed in, or otherwise relevant to a given product or process unless indicated to the contrary or otherwise evident from the context. In some embodiments, exactly one member of a group is present in, employed in, or otherwise relevant to a given product or process. In some embodiments, more than one, or all group members are present in, employed in, or otherwise relevant to a given product or process. It is to be understood that the present disclosure encompasses all variations, combinations, and permutations in which one or more limitations, elements, clauses, descriptive terms, etc., from one or more of the listed claims is introduced into another claim dependent on the same base claim (or, as relevant, any other claim) unless otherwise indicated or unless it would be evident to one of ordinary skill in the art that a contradiction or inconsistency would arise. Where elements are presented as lists (e.g., in Markush group or similar format), it is to be understood that each subgroup of the elements is also disclosed, and any element(s) can be removed from the group. It should be understood that, in general, where embodiments or aspects are referred to as “comprising” particular elements, features, etc., certain embodiments or aspects “consist,” or “consist essentially of,” such elements, features, etc. For purposes of simplicity, those embodiments have not in every case been specifically set forth in so many words herein. It should also be understood that any embodiment or aspect can be explicitly excluded from the claims, regardless of whether the specific exclusion is recited in the specification.

Throughout the specification, whenever a polynucleotide or polypeptide is represented by a sequence of letters (e.g., A, C, G, and T, which denote adenosine, cytidine, guanosine, and thymidine, respectively in the case of a polynucleotide), such polynucleotides or polypeptides are presented in 5′ to 3′ or N-terminus to C-terminus order, from left to right.

Administration: As used herein, the term “administration” typically refers to administration of a composition to a subject or system to achieve delivery of an agent to a subject or system. In some embodiments, an agent is, or is included in, a composition; in some embodiments, an agent is generated through metabolism of a composition or one or more components thereof. Those of ordinary skill in the art will be aware of a variety of routes that may, in appropriate circumstances, be utilized for administration to a subject, for example a human. For example, in some embodiments, administration may be systematic or local. In some embodiments, a systematic administration can be intravenous. In some embodiments, administration can be local. Local administration can involve delivery to cochlear perilymph via, e.g., injection through a round-window membrane or into scala-tympani, a scala-media injection through endolymph, perilymph and/or endolymph following canalostomy. In some embodiments, administration may involve only a single dose. In some embodiments, administration may involve application of a fixed number of doses. In some embodiments, administration may involve dosing that is intermittent (e.g., a plurality of doses separated in time) and/or periodic (e.g., individual doses separated by a common period of time) dosing. In some embodiments, administration may involve continuous dosing (e.g., perfusion) for at least a selected period of time.

Allele: As used herein, the term “allele” refers to one of two or more existing genetic variants of a specific polymorphic genomic locus.

Amelioration: As used herein, the term “amelioration” refers to prevention, reduction or palliation of a state, or improvement of a state of a subject. Amelioration may include, but does not require, complete recovery or complete prevention of a disease, disorder or condition.

Amino acid: In its broadest sense, as used herein, the term “amino acid” refers to any compound and/or substance that can be incorporated into a polypeptide chain, e.g., through formation of one or more peptide bonds. In some embodiments, an amino acid has a general structure, e.g., H₂N—C(H)(R)—COOH. In some embodiments, an amino acid is a naturally-occurring amino acid. In some embodiments, an amino acid is a non-natural amino acid; in some embodiments, an amino acid is a D-amino acid; in some embodiments, an amino acid is an L-amino acid. “Standard amino acid” refers to any of the twenty standard L-amino acids commonly found in naturally occurring peptides. “Nonstandard amino acid” refers to any amino acid, other than standard amino acids, regardless of whether it is prepared synthetically or obtained from a natural source. In some embodiments, an amino acid, including a carboxy- and/or amino-terminal amino acid in a polypeptide, can contain a structural modification as compared with general structure as shown above. For example, in some embodiments, an amino acid may be modified by methylation, amidation, acetylation, pegylation, glycosylation, phosphorylation, and/or substitution (e.g., of an amino group, a carboxylic acid group, one or more protons, and/or a hydroxyl group) as compared with a general structure. In some embodiments, such modification may, for example, alter circulating half-life of a polypeptide containing a modified amino acid as compared with one containing an otherwise identical unmodified amino acid. In some embodiments, such modification does not significantly alter a relevant activity of a polypeptide containing a modified amino acid, as compared with one containing an otherwise identical unmodified amino acid.

Approximately or About: As used herein, the terms “approximately” or “about” may be applied to one or more values of interest, including a value that is similar to a stated reference value. In some embodiments, the term “approximately” or “about” refers to a range of values that fall within ±10% (greater than or less than) of a stated reference value unless otherwise stated or otherwise evident from context (except where such number would exceed 100% of a possible value). For example, in some embodiments, the term “approximately” or “about” may encompass a range of values that within 10%, 9%, 8%, 7%, 6%, 5%, 4%, 3%, 2%, 1%, or less of a reference value.

Associated: As used herein, the term “associated” describes two events or entities as “associated” with one another, if the presence, level and/or form of one is correlated with that of the other. For example, a particular entity (e.g., polypeptide, genetic signature, metabolite, microbe, etc.) is considered to be associated with a particular disease, disorder, or condition, if its presence, level and/or form correlates with incidence of and/or susceptibility to the disease, disorder, or condition (e.g., across a relevant population). In some embodiments, two or more entities are physically “associated” with one another if they interact, directly or indirectly, so that they are and/or remain in physical proximity with one another. In some embodiments, two or more entities that are physically associated with one another are covalently linked to one another; in some embodiments, two or more entities that are physically associated with one another are not covalently linked to one another but are non-covalently associated, for example by means of hydrogen bonds, van der Waals interaction, hydrophobic interactions, magnetism, and combinations thereof.

Biologically active: As used herein, the term “biologically active” refers to an observable biological effect or result achieved by an agent or entity of interest. For example, in some embodiments, a specific binding interaction is a biological activity. In some embodiments, modulation (e.g., induction, enhancement, or inhibition) of a biological pathway or event is a biological activity. In some embodiments, presence or extent of a biological activity is assessed through detection of a direct or indirect product produced by a biological pathway or event of interest.

Cell Selective Promoter: As used herein, the term “cell selective promoter” refers to a promoter that is predominately active in certain cell types (e.g., transcription of a specific gene occurs only within cells expressing transcription regulatory and/or control proteins that bind to the tissue-specific promoter). In some aspects, an inner ear supporting cell selective promoter is a promoter that is predominately active in one or more supporting cells of the inner ear.

Characteristic portion: As used herein, the term “characteristic portion,” in the broadest sense, refers to a portion of a substance whose presence (or absence) correlates with presence (or absence) of a particular feature, attribute, or activity of the substance. In some embodiments, a characteristic portion of a substance is a portion that is found in a given substance and in related substances that share a particular feature, attribute or activity, but not in those that do not share the particular feature, attribute or activity. In some embodiments, a characteristic portion shares at least one functional characteristic with the intact substance. For example, in some embodiments, a “characteristic portion” of a protein or polypeptide is one that contains a continuous stretch of amino acids, or a collection of continuous stretches of amino acids, that together are characteristic of a protein or polypeptide. In some embodiments, each such continuous stretch generally contains at least 2, 5, 10, 15, 20, 50, or more amino acids. In general, a characteristic portion of a substance (e.g., of a protein, antibody, etc.) is one that, in addition to a sequence and/or structural identity specified above, shares at least one functional characteristic with the relevant intact substance. In some embodiments, a characteristic portion may be biologically active.

Characteristic sequence: As used herein, the term “characteristic sequence” is a sequence that is found in all members of a family of polypeptides or nucleic acids, and therefore can be used by those of ordinary skill in the art to define members of the family.

Characteristic sequence element: As used herein, the phrase “characteristic sequence element” refers to a sequence element found in a polymer (e.g., in a polypeptide or nucleic acid) that represents a characteristic portion of that polymer. In some embodiments, presence of a characteristic sequence element correlates with presence or level of a particular activity or property of a polymer. In some embodiments, presence (or absence) of a characteristic sequence element defines a particular polymer as a member (or not a member) of a particular family or group of such polymers. A characteristic sequence element typically comprises at least two monomers (e.g., amino acids or nucleotides). In some embodiments, a characteristic sequence element includes at least 2, 3, 4, 5, 6, 7, 8, 9, 10, 11, 12, 13, 14, 15, 20, 25, 30, 35, 40, 45, 50, or more monomers (e.g., contiguously linked monomers). In some embodiments, a characteristic sequence element includes at least first and second stretches of contiguous monomers spaced apart by one or more spacer regions whose length may or may not vary across polymers that share a sequence element.

Combination therapy: As used herein, the term “combination therapy” refers to those situations in which a subject is simultaneously exposed to two or more therapeutic regimens (e.g., two or more therapeutic agents). In some embodiments, two or more agents may be administered simultaneously. In some embodiments, two or more agents may be administered sequentially. In some embodiments, two or more agents may be administered in overlapping dosing regimens.

Comparable: As used herein, the term “comparable” refers to two or more agents, entities, situations, sets of conditions, subjects, populations, etc., that may not be identical to one another but that are sufficiently similar to permit comparison therebetween so that one skilled in the art will appreciate that conclusions may reasonably be drawn based on differences or similarities observed. In some embodiments, comparable sets of agents, entities, situations, sets of conditions, subjects, populations, etc. are characterized by a plurality of substantially identical features and one or a small number of varied features. Those of ordinary skill in the art will understand, in context, what degree of identity is required in any given circumstance for two or more such agents, entities, situations, sets of conditions, subjects, populations, etc. to be considered comparable. For example, those of ordinary skill in the art will appreciate that sets of agents, entities, situations, sets of conditions, subjects, populations, etc. are comparable to one another when characterized by a sufficient number and type of substantially identical features to warrant a reasonable conclusion that differences in results obtained or phenomena observed under or with different sets of circumstances, stimuli, agents, entities, situations, sets of conditions, subjects, populations, etc. are caused by or indicative of the variation in those features that are varied.

Construct: As used herein, the term “construct” refers to a composition including a polynucleotide capable of carrying at least one heterologous polynucleotide. In some embodiments, a construct can be a plasmid, a transposon, a cosmid, an artificial chromosome (e.g., a human artificial chromosome (HAC), a yeast artificial chromosome (YAC), a bacterial artificial chromosome (BAC), or a P1-derived artificial chromosome (PAC)) or a viral construct, and any Gateway® plasmids. A construct can, e.g., include sufficient cis-acting elements for expression; other elements for expression can be supplied by the host primate cell or in an in vitro expression system. A construct may include any genetic element (e.g., a plasmid, a transposon, a cosmid, an artificial chromosome, or a viral construct, etc.) that is capable of replicating when associated with proper control elements. Thus, in some embodiments, “construct” may include a cloning and/or expression construct and/or a viral construct (e.g., an adeno-associated virus (AAV) construct, an adenovirus construct, a lentivirus construct, or a retrovirus construct).

Conservative: As used herein, the term “conservative” refers to instances describing a conservative amino acid substitution, including a substitution of an amino acid residue by another amino acid residue having a side chain R group with similar chemical properties (e.g., charge or hydrophobicity). In general, a conservative amino acid substitution will not substantially change functional properties of interest of a protein, for example, ability of a receptor to bind to a ligand. Examples of groups of amino acids that have side chains with similar chemical properties include: aliphatic side chains such as glycine (Gly, G), alanine (Ala, A), valine (Val, V), leucine (Leu, L), and isoleucine (Ile, I); aliphatic-hydroxyl side chains such as serine (Ser, S) and threonine (Thr, T); amide-containing side chains such as asparagine (Asn, N) and glutamine (Gln, Q); aromatic side chains such as phenylalanine (Phe, F), tyrosine (Tyr, Y), and tryptophan (Trp, W); basic side chains such as lysine (Lys, K), arginine (Arg, R), and histidine (His, H); acidic side chains such as aspartic acid (Asp, D) and glutamic acid (Glu, E); and sulfur-containing side chains such as cysteine (Cys, C) and methionine (Met, M). Conservative amino acids substitution groups include, for example, valine/leucine/soleucine (Val/Leu/Ile, V/UI), phenylalanine/tyrosine (Phe/Tyr, F/Y), lysine/arginine (Lys/Arg, K/R), alanine/valine (Ala/Val, A/V), glutamate/aspartate (Glu/Asp, E/D), and asparagine/glutamine (Asn/Gln, N/Q). In some embodiments, a conservative amino acid substitution can be a substitution of any native residue in a protein with alanine, as used in, for example, alanine scanning mutagenesis. In some embodiments, a conservative substitution is made that has a positive value in the PAM250 log-likelihood matrix disclosed in Gonnet et al., 1992, Science 256:1443-1445, which is incorporated herein by reference in its entirety. In some embodiments, a substitution is a moderately conservative substitution wherein the substitution has a nonnegative value in the PAM250 log-likelihood matrix. One skilled in the art would appreciate that a change (e.g., substitution, addition, deletion, etc.) of amino acids that are not conserved between the same protein from different species is less likely to have an effect on the function of a protein and therefore, these amino acids should be selected for mutation. Amino acids that are conserved between the same protein from different species should not be changed (e.g., deleted, added, substituted, etc.), as these mutations are more likely to result in a change in function of a protein.

CONSERVATIVE AMINO ACID SUBSTITUTIONS

| For Amino Acid | Code | Replace With |
|---|---|---|
| Alanine | A | D-ala, Gly, Aib, β-Ala, Acp, L-Cys, D-Cys |
| Arginine | R | D-Arg, Lys, D-Lys, homo-Arg, D-homo-Arg, |
| | | Met, Ile, D-Met, D-Ile, Orn, D-Orn |
| Asparagine | N | D-Asn, Asp, D-Asp, Glu, D-Glu, Gln, D-Gln |
| Aspartic Acid | D | D-Asp, D-Asn, Asn, Glu, D-Glu, Gln, D-Gln |
| Cysteine | C | D-Cys, S—Me-Cys, Met, D-Met, Thr, D-Thr |
| Glutamine | Q | D-Gln, Asn, D-Asn, Glu, D-Glu, Asp, D-Asp |
| Glutamic Acid | E | D-Glu, D-Asp, Asp, Asn, D-Asn, Gln, D-Gln |
| Glycine | G | Ala, D-Ala, Pro, D-Pro, Aib, β-Ala, Acp |
| Isoleucine | I | D-Ile, Val, D-Val, AdaA, AdaG, Leu, D-Leu, |
| | | Met, D-Met |
| Leucine | L | D-Leu, Val, D-Val, AdaA, AdaG, Leu, D-Leu, |
| | | Met, D-Met |
| Lysine | K | D-Lys, Arg, D-Arg, homo-Arg, D-homo-Arg, |
| | | Met, D-Met, Ile, D-Ile, Orn, D-Orn |
| Methionine | M | D-Met, S—Me-Cys, Ile, D-Ile, Leu, D-Leu, |
| | | Val, D-Val |
| Phenylalanine | F | D-Phe, Tyr, D-Thr, L-Dopa, His, D-His, Trp, |
| | | D-Trp, Trans-3,4 or 5-phenylproline, AdaA, |
| | | AdaG, cis-3,4 or 5-phenylproline, Bpa, D-Bpa |
| Proline | P | D-Pro, L-I-thioazolidine-4-carboxylic acid, |
| | | D-or-L-1-oxazolidine-4-carboxylic acid (Kauer, |
| | | U.S. Pat. No. 4,511,390) |
| Serine | S | D-Ser, Thr, D-Thr, allo-Thr, Met, D-Met, Met |
| | | (O), D-Met (O), L-Cys, D-Cys |
| Threonine | T | D-Thr, Ser, D-Ser, allo-Thr, Met, D-Met, Met |
| | | (O), D-Met (O), Val, D-Val |
| Tyrosine | Y | D-Tyr, Phe, D-Phe, L-Dopa, His, D-His |
| Valine | V | D-Val, Leu, D-Leu, Ile, D-Ile, Met, D-Met, |
| | | AdaA, AdaG |

Control: As used herein, the term “control” refers to the art-understood meaning of a “control” being a standard against which results are compared. Typically, controls are used to augment integrity in experiments by isolating variables in order to make a conclusion about such variables. In some embodiments, a control is a reaction or assay that is performed simultaneously with a test reaction or assay to provide a comparator. For example, in one experiment, a “test” (i.e., a variable being tested) is applied. In a second experiment, a “control,” the variable being tested is not applied. In some embodiments, a control is a historical control (e.g., of a test or assay performed previously, or an amount or result that is previously known). In some embodiments, a control is or comprises a printed or otherwise saved record. In some embodiments, a control is a positive control. In some embodiments, a control is a negative control.

Determining, measuring, evaluating, assessing, assaying and analyzing: As used herein, the terms “determining,” “measuring,” “evaluating,” “assessing,” “assaying,” and “analyzing” may be used interchangeably to refer to any form of measurement, and include determining if an element is present or not. These terms include both quantitative and/or qualitative determinations. Assaying may be relative or absolute. For example, in some embodiments, “Assaying for the presence of” can be determining an amount of something present and/or determining whether or not it is present or absent.

Engineered: In general, as used herein, the term “engineered” refers to an aspect of having been manipulated by the hand of man. For example, a cell or organism is considered to be “engineered” if it has been manipulated so that its genetic information is altered (e.g., new genetic material not previously present has been introduced, for example by transformation, mating, somatic hybridization, transfection, transduction, or other mechanism, or previously present genetic material is altered or removed, for example by substitution or deletion mutation, or by mating protocols). As is common practice and is understood by those in the art, progeny of an engineered polynucleotide or cell are typically still referred to as “engineered” even though the actual manipulation was performed on a prior entity.

Excipient: As used herein, the term “excipient” refers to an inactive (e.g., non-therapeutic) agent that may be included in a pharmaceutical composition, for example to provide or contribute to a desired consistency or stabilizing effect. In some embodiments, suitable pharmaceutical excipients may include, for example, starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like.

Expression: As used herein, the term “expression” of a nucleic acid sequence refers to generation of any gene product (e.g., transcript, e.g., mRNA, e.g., polypeptide, etc.) from a nucleic acid sequence. In some embodiments, a gene product can be a transcript. In some embodiments, a gene product can be a polypeptide. In some embodiments, expression of a nucleic acid sequence involves one or more of the following: (1) production of an RNA template from a DNA sequence (e.g., by transcription); (2) processing of an RNA transcript (e.g., by splicing, editing, 5′ cap formation, and/or 3′ end formation); (3) translation of an RNA into a polypeptide or protein; and/or (4) post-translational modification of a polypeptide or protein.

Flanked: As used herein, the term “flanked” refers to a position relative to ends of a reference item. More specifically, in referring to reference nucleic acid sequence(s), “flanked” refers to having sequences upstream and downstream of the reference nucleic acid sequence(s). In some aspects, a flanked referenced nucleic acid sequence has a first sequence or series of nucleotide residues positioned adjacent to the 5′ end of the referenced nucleic acid and a second sequence or series of nucleotide residues positioned adjacent to the 3′ end of the referenced nucleic acid. In some aspects, the upstream and/or downstream flanking sequences are immediately adjacent to the referenced nucleic acid sequence. In some aspects, there are intervening nucleic acids between the upstream and/or downstream flanking sequences and the referenced nucleic acid sequence.

Functional: As used herein, the term “functional” describes something that exists in a form in which it exhibits a property and/or activity by which it is characterized. For example, in some aspects, a “functional” biological molecule is a biological molecule in a form in which it exhibits a property and/or activity by which it is characterized. In some such aspects, a functional biological molecule is characterized relative to another biological molecule which is non-functional in that the “non-functional” version does not exhibit the same or equivalent property and/or activity as the “functional” molecule. A biological molecule may have one function, two functions (i.e., bifunctional) or many functions (i.e., multifunctional).

Gene: As used herein, the term “gene” refers to a DNA sequence in a chromosome that codes for a gene product (e.g., an RNA product, e.g., a polypeptide product). In some embodiments, a gene includes coding sequence (i.e., sequence that encodes a particular product). In some embodiments, a gene includes non-coding sequence. In some particular embodiments, a gene may include both coding (e.g., exonic) and non-coding (e.g., intronic) sequence. In some embodiments, a gene may include one or more regulatory sequences (e.g., promoters, enhancers, etc.) and/or intron sequences that, for example, may control or impact one or more aspects of gene expression (e.g., cell-type-specific expression, inducible expression, etc.). As used herein, the term “gene” generally refers to a portion of a nucleic acid that encodes a polypeptide or fragment thereof, the term may optionally encompass regulatory sequences, as will be clear from context to those of ordinary skill in the art. This definition is not intended to exclude application of the term “gene” to non-protein-coding expression units but rather to clarify that, in most cases, the term as used in this document refers to a polypeptide-coding nucleic acid. In some embodiments, a gene may encode a polypeptide, but that polypeptide may not be functional, e.g., a gene variant may encode a polypeptide that does not function in the same way, or at all, relative to the wild-type gene. In some embodiments, a gene may encode a transcript which, in some embodiments, may be toxic beyond a threshold level. In some embodiments, a gene may encode a polypeptide, but that polypeptide may not be functional and/or may be toxic beyond a threshold level.

Hearing loss: As used herein, the term “hearing loss” may be used to a partial or total inability of a living organism to hear. In some embodiments, hearing loss may be acquired. In some embodiments, hearing loss may be hereditary. In some embodiments, hearing loss may be genetic. In some embodiments, hearing loss may be as a result of disease or trauma (e.g., physical trauma, treatment with one or more agents resulting in hearing loss, etc.). In some embodiments, hearing loss may be due to one or more known genetic causes and/or syndromes. In some embodiments, hearing loss may be of unknown etiology. In some embodiments, hearing loss may or may not be mitigated by use of hearing aids or other treatments.

Heterologous: As used herein, the term “heterologous” may be used in reference to one or more regions of a particular molecule as compared to another region and/or another molecule. In some embodiments, heterologous polypeptide domains, refers to the fact that polypeptide domains do not naturally occur together (e.g., in the same polypeptide). For example, in fusion proteins generated by the hand of man, a polypeptide domain from one polypeptide may be fused to a polypeptide domain from a different polypeptide. In such a fusion protein, two polypeptide domains would be considered “heterologous” with respect to each other, as they do not naturally occur together.

Identity: As used herein, the term “identity” refers to overall relatedness between polymeric molecules, e.g., between nucleic acid molecules (e.g., DNA molecules and/or RNA molecules) and/or between polypeptide molecules. In some embodiments, polymeric molecules are considered to be “substantially identical” to one another if their sequences are at least 25%, 30%, 35%, 40%, 45%, 50%, 55%, 60%, 65%, 70%, 75%, 80%, 85%, 90%, 95%, or 99% identical. Calculation of percent identity of two nucleic acid or polypeptide sequences, for example, can be performed by aligning two sequences for optimal comparison purposes (e.g., gaps can be introduced in one or both of a first and a second sequences for optimal alignment and non-identical sequences can be disregarded for comparison purposes). In some embodiments, a length of a sequence aligned for comparison purposes is at least 30%, at least 40%, at least 50%, at least 60%, at least 70%, at least 80%, at least 90%, at least 95%, or substantially 100% of length of a reference sequence; nucleotides at corresponding positions are then compared. When a position in the first sequence is occupied by the same residue (e.g., nucleotide or amino acid) as a corresponding position in the second sequence, then the two molecules (i.e., first and second) are identical at that position. Percent identity between two sequences is a function of the number of identical positions shared by the two sequences being compared, taking into account the number of gaps, and the length of each gap, which needs to be introduced for optimal alignment of the two sequences. Comparison of sequences and determination of percent identity between two sequences can be accomplished using a mathematical algorithm. For example, percent identity between two nucleotide sequences can be determined using the algorithm of Meyers and Miller (CABIOS, 1989, 4: 11-17, which is herein incorporated by reference in its entirety), which has been incorporated into the ALIGN program (version 2.0). In some embodiments, nucleic acid sequence comparisons made with the ALIGN program use a PAM120 weight residue table, a gap length penalty of 12 and a gap penalty of 4.

Inhibitory nucleic acid: As used herein, the term “inhibitory nucleic acid” refers to a nucleic acid sequence that hybridizes specifically to a target gene, including target DNA or RNA (e.g., a target mRNA (e.g., a connexin gene product, e.g., a connexin mRNA, e.g., GJB2 mRNA)). Thereby, in some embodiments, an inhibitory nucleic acid inhibits expression and/or activity of a target gene. In some embodiments, an inhibitory nucleic acid is a short interfering RNA (siRNA), a short hairpin RNA (shRNA), a microRNA (miRNA), an antisense oligonucleotide, a guide RNA (gRNA), or a ribozyme. In some embodiments, an inhibitory nucleic acid is between about 10 nucleotides to about 30 nucleotides in length (e.g., about 10 nucleotides to about 28 nucleotides, about 10 nucleotides to about 26 nucleotides, about 10 nucleotides to about 24 nucleotides, about 10 nucleotides to about 22 nucleotides, about 10 nucleotides to about 20 nucleotides, about 10 nucleotides to about 18 nucleotides, about 10 nucleotides to about 16 nucleotides, about 10 nucleotides to about 14 nucleotides, about 10 nucleotides to about 12 nucleotides, about 12 nucleotides to about 30 nucleotides, about 12 nucleotides to about 28 nucleotides, about 12 nucleotides to about 26 nucleotides, about 12 nucleotides to about 24 nucleotides, about 12 nucleotides to about 22 nucleotides, about 12 nucleotides to about 20 nucleotides, about 12 nucleotides to about 18 nucleotides, about 12 nucleotides to about 16 nucleotides, about 12 nucleotides to about 14 nucleotides, about 16 nucleotides to about 30 nucleotides, about 16 nucleotides to about 28 nucleotides, about 16 nucleotides to about 26 nucleotides, about 16 nucleotides to about 24 nucleotides, about 16 nucleotides to about 22 nucleotides, about 16 nucleotides to about 20 nucleotides, about 16 nucleotides to about 18 nucleotides, about 18 nucleotides to about 30 nucleotides, about 18 nucleotides to about 28 nucleotides, about 18 nucleotides to about 26 nucleotides, about 18 nucleotides to about 24 nucleotides, about 18 nucleotides to about 22 nucleotides, about 18 nucleotides to about 20 nucleotides, about 20 nucleotides to about 30 nucleotides, about 20 nucleotides to about 28 nucleotides, about 20 nucleotides to about 26 nucleotides, about 20 nucleotides to about 24 nucleotides, about 20 nucleotides to about 22 nucleotides, about 22 nucleotides to about 30 nucleotides, about 22 nucleotides to about 28 nucleotides, about 22 nucleotides to about 26 nucleotides, about 22 nucleotides to about 24 nucleotides, about 24 nucleotides to about 30 nucleotides, about 24 nucleotides to about 28 nucleotides, about 24 nucleotides to about 26 nucleotides, about 26 nucleotides to about 30 nucleotides, about 26 nucleotides to about 28 nucleotides, about 28 nucleotides to about 30 nucleotides, or 11, 12, 13, 14, 15, 16, 17, 18, 19, 20, 21, 22, 23, 24, 25, 26, 27, 28, 29 or 30 nucleotides). In some embodiments, an inhibitory nucleic acid is an inhibitory RNA that targets GJB2. In some such embodiments, an inhibitory GJB2 RNA hybridizes specifically to a target on an RNA molecule comprising GJB2. In some such embodiments, a GJB2 inhibitory RNA includes, e.g., an inhibitory nucleic acid is a short interfering RNA (siRNA), a short hairpin RNA (shRNA), a microRNA (miRNA), an antisense oligonucleotide, a guide RNA (gRNA), or a ribozyme. In some embodiments, hybridizing of an inhibitory GJB2 RNA reduces expression of a GJB2 gene product. Exemplary inhibitory RNA sequences suitable for GJB2 inhibition are provided herein.

Improve, increase, enhance, inhibit or reduce: As used herein, the terms “improve,” “increase,” “enhance,” “inhibit,” “reduce,” or grammatical equivalents thereof, indicate values that are relative to a baseline or other reference measurement. In some embodiments, a value is statistically significantly difference that a baseline or other reference measurement. In some embodiments, an appropriate reference measurement may be or comprise a measurement in a particular system (e.g., in a single individual) under otherwise comparable conditions absent presence of (e.g., prior to and/or after) a particular agent or treatment, or in presence of an appropriate comparable reference agent. In some embodiments, an appropriate reference measurement may be or comprise a measurement in comparable system known or expected to respond in a particular way, in presence of the relevant agent or treatment. In some embodiments, an appropriate reference is a negative reference; in some embodiments, an appropriate reference is a positive reference.

Knockdown: As used herein, the term “knockdown” refers to a decrease in expression of one or more gene products. In some embodiments, an inhibitory nucleic acid achieve knockdown. In some embodiments, a genome editing system described herein achieves knockdown.

Knockout: As used herein, the term “knockout” refers to ablation of expression of one or more gene products. In some embodiments, a genome editing system described herein achieve knockout.

microRNA: As used herein, the term “microRNA” or “miRNA” refer to a class of biomolecules involved in control of gene expression. A mature miRNA is typically an 18-25 nucleotide non-coding RNA that regulates expression of an mRNA including sequences complementary to the miRNA. These small RNA molecules are known to control gene expression by regulating the stability and/or translation of mRNAs. In some aspects, a miRNAs binds to the 3′ UTR of target mRNAs and suppress translation. MiRNAs can also bind to target mRNAs and mediate gene silencing through the RNAi pathway. MiRNAs can also regulate gene expression by causing chromatin condensation.

In some aspects, a microRNA is between about 10 nucleotides to about 30 nucleotides in length (e.g., about 10 nucleotides to about 28 nucleotides, about 10 nucleotides to about 26 nucleotides, about 10 nucleotides to about 24 nucleotides, about 10 nucleotides to about 22 nucleotides, about 10 nucleotides to about 20 nucleotides, about 10 nucleotides to about 18 nucleotides, about 10 nucleotides to about 16 nucleotides, about 10 nucleotides to about 14 nucleotides, about 10 nucleotides to about 12 nucleotides, about 12 nucleotides to about 30 nucleotides, about 12 nucleotides to about 28 nucleotides, about 12 nucleotides to about 26 nucleotides, about 12 nucleotides to about 24 nucleotides, about 12 nucleotides to about 22 nucleotides, about 12 nucleotides to about 20 nucleotides, about 12 nucleotides to about 18 nucleotides, about 12 nucleotides to about 16 nucleotides, about 12 nucleotides to about 14 nucleotides, about 16 nucleotides to about 30 nucleotides, about 16 nucleotides to about 28 nucleotides, about 16 nucleotides to about 26 nucleotides, about 16 nucleotides to about 24 nucleotides, about 16 nucleotides to about 22 nucleotides, about 16 nucleotides to about 20 nucleotides, about 16 nucleotides to about 18 nucleotides, about 18 nucleotides to about 30 nucleotides, about 18 nucleotides to about 28 nucleotides, about 18 nucleotides to about 26 nucleotides, about 18 nucleotides to about 24 nucleotides, about 18 nucleotides to about 22 nucleotides, about 18 nucleotides to about 20 nucleotides, about 20 nucleotides to about 30 nucleotides, about 20 nucleotides to about 28 nucleotides, about 20 nucleotides to about 26 nucleotides, about 20 nucleotides to about 24 nucleotides, about 20 nucleotides to about 22 nucleotides, about 22 nucleotides to about 30 nucleotides, about 22 nucleotides to about 28 nucleotides, about 22 nucleotides to about 26 nucleotides, about 22 nucleotides to about 24 nucleotides, about 24 nucleotides to about 30 nucleotides, about 24 nucleotides to about 28 nucleotides, about 24 nucleotides to about 26 nucleotides, about 26 nucleotides to about 30 nucleotides, about 26 nucleotides to about 28 nucleotides, about 28 nucleotides to about 30 nucleotides, or 11, 12, 13, 14, 15, 16, 17, 18, 19, 20, 21, 22, 23, 24, 25, 26, 27, 28, 29 or 30 nucleotides).

microRNA regulatory target site: As used herein, the term “microRNA regulatory target site” or “miRTS” refers to a sequence that directly interacts with a miRNA on the mRNA transcript. Often, the miRTS is present in the 3′ untranslated region (UTR) of the mRNA, but it may also be present in the coding sequence, or in the 5′ UTR. miRTS are not necessarily perfect complements to miRNAs, usually having only a few bases of complementarity to the miRNA, and often containing one or more mismatches. The miRTS may be any sequence capable of being bound by a miRNA sufficiently that the translation of a gene to which the miRTS is operably linked is repressed by a miRNA silencing mechanism such as the RNA-induced silencing complex (RISC). In some aspects, inclusion of a miRTS into a nucleic acid construct comprising a polynucleotide (e.g., a therapeutic polynucleotide) can result in degradation of the therapeutic polynucleotide after transcription. In some aspects, inclusion of a miRTS into a nucleic acid construct comprising a polynucleotide (e.g., a therapeutic polynucleotide) can result in decreased expression of the therapeutic polynucleotide in cells expressing the miRNA.

Nucleic acid: As used herein, the term “nucleic acid”, in its broadest sense, refers to any compound and/or substance that is or can be incorporated into an oligonucleotide chain. In some embodiments, a nucleic acid is a compound and/or substance that is or can be incorporated into an oligonucleotide chain via a phosphodiester linkage. As will be clear from context, in some embodiments, “nucleic acid” refers to an individual nucleic acid residue (e.g., a nucleotide and/or nucleoside); in some embodiments, “nucleic acid” refers to an oligonucleotide chain comprising individual nucleic acid residues. In some embodiments, a “nucleic acid” is or comprises RNA; in some embodiments, a “nucleic acid” is or comprises DNA. In some embodiments, a nucleic acid is, comprises, or consists of one or more natural nucleic acid residues. In some embodiments, a nucleic acid is, comprises, or consists of one or more nucleic acid analogs. In some embodiments, a nucleic acid analog differs from a nucleic acid in that it does not utilize a phosphodiester backbone. Alternatively or additionally, in some embodiments, a nucleic acid has one or more phosphorothioate and/or 5′-N-phosphoramidite linkages rather than phosphodiester bonds. In some embodiments, a nucleic acid is, comprises, or consists of one or more natural nucleosides (e.g., adenosine, thymidine, guanosine, cytidine, uridine, deoxyadenosine, deoxythymidine, deoxy guanosine, and deoxycytidine). In some embodiments, a nucleic acid is, comprises, or consists of one or more nucleoside analogs (e.g., 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, O(6)-methylguanine, 2-thiocytidine, methylated bases, intercalated bases, and combinations thereof). In some embodiments, a nucleic acid comprises one or more modified sugars (e.g., 2′-fluororibose, ribose, 2′-deoxyribose, arabinose, and hexose) as compared with those in natural nucleic acids. In some embodiments, a nucleic acid has a nucleotide sequence that encodes a functional gene product such as an RNA or protein. In some embodiments, a nucleic acid includes one or more introns. In some embodiments, nucleic acids are prepared by one or more of isolation from a natural source, enzymatic synthesis by polymerization based on a complementary template (in vivo or in vitro), reproduction in a recombinant cell or system, and chemical synthesis. In some embodiments, a nucleic acid is at least 3, 4, 5, 6, 7, 8, 9, 10, 15, 20, 25, 30, 35, 40, 45, 50, 55, 60, 65, 70, 75, 80, 85, 90, 95, 100, 110, 120, 130, 140, 150, 160, 170, 180, 190, 20, 225, 250, 275, 300, 325, 350, 375, 400, 425, 450, 475, 500, 600, 700, 800, 900, 1000, 1500, 2000, 2500, 3000, 3500, 4000, 4500, 5000 or more residues long. In some embodiments, a nucleic acid is partly or wholly single stranded; in some embodiments, a nucleic acid is partly or wholly double stranded. In some embodiments, a nucleic acid has a nucleotide sequence comprising at least one element that encodes, or is complementary to a sequence that encodes, a polypeptide. In some embodiments, a nucleic acid has enzymatic activity.

Operably linked: As used herein, refers to a juxtaposition wherein the components described are in a relationship permitting them to function in their intended manner. A control element “operably linked” to a functional element is associated in such a way that expression and/or activity of the functional element is achieved under conditions compatible with the control element. In some embodiments, “operably linked” control elements are contiguous (e.g., covalently linked) with coding elements of interest; in some embodiments, control elements act in trans to or otherwise at a from the functional element of interest. In some embodiments, “operably linked” refers to functional linkage between a regulatory sequence and a heterologous nucleic acid sequence resulting in expression of the latter. For example, a first nucleic acid sequence is operably linked with a second nucleic acid sequence when the first nucleic acid sequence is placed in a functional relationship with the second nucleic acid sequence. In some embodiments, for example, a functional linkage may include transcriptional control. For instance, a promoter is operably linked to a coding sequence if the promoter affects the transcription or expression of the coding sequence. Operably linked DNA sequences can be contiguous with each other and, e.g., where necessary to join two protein coding regions, are in the same reading frame.

Pharmaceutical composition: As used herein, the term “pharmaceutical composition” refers to a composition in which an active agent is formulated together with one or more pharmaceutically acceptable carriers. In some embodiments, an active agent is present in unit dose amount appropriate for administration in a therapeutic regimen that shows a statistically significant probability of achieving a predetermined therapeutic effect when administered to a relevant population. In some embodiments, a pharmaceutical composition may be specially formulated for administration in solid or liquid form, including those adapted for, e.g., administration, for example, an injectable formulation that is, e.g., an aqueous or non-aqueous solution or suspension or a liquid drop designed to be administered into an ear canal. In some embodiments, a pharmaceutical composition may be formulated for administration via injection either in a particular organ or compartment, e.g., directly into an ear, or systemic, e.g., intravenously. In some embodiments, a formulation may be or comprise drenches (aqueous or non-aqueous solutions or suspensions), tablets, boluses, powders, granules, pastes, capsules, powders, etc. In some embodiments, an active agent may be or comprise an isolated, purified, or pure compound.

Pharmaceutically acceptable: As used herein, the term “pharmaceutically acceptable” which, for example, may be used in reference to a carrier, diluent, or excipient used to formulate a pharmaceutical composition as disclosed herein, means that a carrier, diluent, or excipient is compatible with other ingredients of a composition and not deleterious to a recipient thereof.

Pharmaceutically acceptable carrier: As used herein, the term “pharmaceutically acceptable carrier” means a pharmaceutically-acceptable material, composition or vehicle, such as a liquid or solid filler, diluent, excipient, or solvent encapsulating material, involved in carrying or transporting a subject compound from one organ, or portion of a body, to another organ, or portion of a body. Each carrier must be is “acceptable” in the sense of being compatible with other ingredients of a formulation and not injurious to a patient. Some examples of materials which can serve as pharmaceutically-acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; pH buffered solutions; polyesters, polycarbonates and/or polyanhydrides; and other non-toxic compatible substances employed in pharmaceutical formulations.

Polyadenylation: As used herein, “polyadenylation” refers to the covalent linkage of a polyadenylyl moiety, or its modified variant, to a messenger RNA molecule. In eukaryotic organisms, most messenger RNA (mRNA) molecules are polyadenylated at the 3′ end. In some embodiments, a 3′ poly(A) tail is a long sequence of adenine nucleotides (e.g., 50, 60, 70, 100, 200, 500, 1000, 2000, 3000, 4000, or 5000) added to the pre-mRNA through the action of an enzyme, polyadenylate polymerase. In higher eukaryotes, a poly(A) tail can be added onto transcripts that contain a specific sequence, the polyadenylation signal or “poly(A) sequence.” A poly(A) tail and proteins bound to it aid in protecting mRNA from degradation by exonucleases. Polyadenylation can be affect transcription termination, export of the mRNA from the nucleus, and translation. Typically, polyadenylation occurs in the nucleus immediately after transcription of DNA into RNA, but additionally can also occur later in the cytoplasm. After transcription has been terminated, the mRNA chain can be cleaved through the action of an endonuclease complex associated with RNA polymerase. The cleavage site can be characterized by the presence of the base sequence AAUAAA near the cleavage site. After mRNA has been cleaved, adenosine residues can be added to the free 3′ end at the cleavage site. As used herein, a “poly(A) sequence” is a sequence that triggers the endonuclease cleavage of an mRNA and the additional of a series of adenosines to the 3′ end of the cleaved mRNA.

Polypeptide: As used herein, the term “polypeptide” refers to any polymeric chain of residues (e.g., amino acids) that are typically linked by peptide bonds. In some embodiments, a polypeptide has an amino acid sequence that occurs in nature. In some embodiments, a polypeptide has an amino acid sequence that does not occur in nature. In some embodiments, a polypeptide has an amino acid sequence that is engineered in that it is designed and/or produced through action of the hand of man. In some embodiments, a polypeptide may comprise or consist of natural amino acids, non-natural amino acids, or both. In some embodiments, a polypeptide may include one or more pendant groups or other modifications, e.g., modifying or attached to one or more amino acid side chains, at a polypeptide's N-terminus, at a polypeptide's C-terminus, or any combination thereof. In some embodiments, such pendant groups or modifications may be acetylation, amidation, lipidation, methylation, pegylation, etc., including combinations thereof. In some embodiments, polypeptides may contain L-amino acids, D-amino acids, or both and may contain any of a variety of amino acid modifications or analogs known in the art. In some embodiments, useful modifications may be or include, e.g., terminal acetylation, amidation, methylation, etc. In some embodiments, a protein may comprise natural amino acids, non-natural amino acids, synthetic amino acids, and combinations thereof. The term “peptide” is generally used to refer to a polypeptide having a length of less than about 100 amino acids, less than about 50 amino acids, less than 20 amino acids, or less than 10 amino acids. In some embodiments, a protein is antibodies, antibody fragments, biologically active portions thereof, and/or characteristic portions thereof.

Polynucleotide: As used herein, the term “polynucleotide” refers to any polymeric chain of nucleic acids. In some embodiments, a polynucleotide is or comprises RNA; in some embodiments, a polynucleotide is or comprises DNA. In some embodiments, a polynucleotide is, comprises, or consists of one or more natural nucleic acid residues. In some embodiments, a polynucleotide is, comprises, or consists of one or more nucleic acid analogs. In some embodiments, a polynucleotide analog differs from a nucleic acid in that it does not utilize a phosphodiester backbone. Alternatively or additionally, in some embodiments, a polynucleotide has one or more phosphorothioate and/or 5′-N-phosphoramidite linkages rather than phosphodiester bonds. In some embodiments, a polynucleotide is, comprises, or consists of one or more natural nucleosides (e.g., adenosine, thymidine, guanosine, cytidine, uridine, deoxyadenosine, deoxythymidine, deoxy guanosine, and deoxycytidine). In some embodiments, a polynucleotide is, comprises, or consists of one or more nucleoside analogs (e.g., 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, O(6)-methylguanine, 2-thiocytidine, methylated bases, intercalated bases, and combinations thereof). In some embodiments, a polynucleotide comprises one or more modified sugars (e.g., 2′-fluororibose, ribose, 2′-deoxyribose, arabinose, and hexose) as compared with those in natural nucleic acids. In some embodiments, a polynucleotide has a nucleotide sequence that encodes a functional gene product such as an RNA or protein. In some embodiments, a polynucleotide includes one or more introns. In some embodiments, a polynucleotide is prepared by one or more of isolation from a natural source, enzymatic synthesis by polymerization based on a complementary template (in vivo or in vitro), reproduction in a recombinant cell or system, and chemical synthesis. In some embodiments, a polynucleotide is at least 3, 4, 5, 6, 7, 8, 9, 10, 15, 20, 25, 30, 35, 40, 45, 50, 55, 60, 65, 70, 75, 80, 85, 90, 95, 100, 110, 120, 130, 140, 150, 160, 170, 180, 190, 20, 225, 250, 275, 300, 325, 350, 375, 400, 425, 450, 475, 500, 600, 700, 800, 900, 1000, 1500, 2000, 2500, 3000, 3500, 4000, 4500, 5000 or more residues long. In some embodiments, a polynucleotide is partly or wholly single stranded; in some embodiments, a polynucleotide is partly or wholly double stranded. In some embodiments, a polynucleotide has a nucleotide sequence comprising at least one element that encodes, or is the complement of a sequence that encodes, a polypeptide. In some embodiments, a polynucleotide has enzymatic activity.

Promoter: As used herein, the term “promoter” refers to a nucleic acid sequence that functions to control the transcription of one or more coding sequences (e.g., a gene or transgene, e.g., encoding a therapeutic polypeptide), located upstream with respect to the direction of transcription of the transcription initiation site of the coding sequence. In some aspects, the promoter is structurally identified by the presence of a binding site for DNA-dependent RNA polymerase, transcription initiation sites or other DNA sequence (e.g., a transcription factor binding site, a repressor and/or activator protein binding site, or other sequences of nucleotides that act directly or indirectly to regulate the amount of transcription from the promoter). In some aspects, the promoter can comprise a naturally occurring promoter sequence, a functional fragment thereof, or a mutant of the naturally occurring promoter sequence or a functional fragment thereof.

Protein: As used herein, the term “protein” refers to a polypeptide (i.e., a string of at least two amino acids linked to one another by peptide bonds). Proteins may include moieties other than amino acids (e.g., may be glycoproteins, proteoglycans, etc.) and/or may be otherwise processed or modified. Those of ordinary skill in the art will appreciate that a “protein” can be a complete polypeptide chain as produced by a cell (with or without a signal sequence), or can be a characteristic portion thereof. Those of ordinary skill will appreciate that a protein can sometimes include more than one polypeptide chain, for example linked by one or more disulfide bonds or associated by other means.

Recombinant: As used herein, the term “recombinant” is intended to refer to polypeptides that are designed, engineered, prepared, expressed, created, manufactured, and/or or isolated by recombinant means, such as polypeptides expressed using a recombinant expression construct transfected into a host cell; polypeptides isolated from a recombinant, combinatorial human polypeptide library; polypeptides isolated from an animal (e.g., a mouse, rabbit, sheep, fish, etc.) that is transgenic for or otherwise has been manipulated to express a gene or genes, or gene components that encode and/or direct expression of the polypeptide or one or more component(s), portion(s), element(s), or domain(s) thereof, and/or polypeptides prepared, expressed, created or isolated by any other means that involves splicing or ligating selected nucleic acid sequence elements to one another, chemically synthesizing selected sequence elements, and/or otherwise generating a nucleic acid that encodes and/or directs expression of a polypeptide or one or more component(s), portion(s), element(s), or domain(s) thereof. In some embodiments, one or more of such selected sequence elements is found in nature. In some embodiments, one or more of such selected sequence elements is designed in silico. In some embodiments, one or more such selected sequence elements results from mutagenesis (e.g., in vivo or in vitro) of a known sequence element, e.g., from a natural or synthetic source such as, for example, in the germline of a source organism of interest (e.g., of a human, a mouse, etc).

Reference: As used herein, the term “reference” describes a standard or control relative to which a comparison is performed. For example, in some embodiments, an agent, animal, individual, population, sample, sequence or value of interest is compared with a reference or control agent, animal, individual, population, sample, sequence or value. In some embodiments, a reference or control is tested and/or determined substantially simultaneously with the testing or determination of interest. In some embodiments, a reference or control is a historical reference or control, optionally embodied in a tangible medium. Typically, as would be understood by those skilled in the art, a reference or control is determined or characterized under comparable conditions or circumstances to those under assessment. Those skilled in the art will appreciate when sufficient similarities are present to justify reliance on and/or comparison to a particular possible reference or control. In some embodiments, a reference is a negative control reference; in some embodiments, a reference is a positive control reference.

Regulatory Element: As used herein, the term “regulatory element” or “regulatory sequence” refers to non-coding regions of DNA that regulate, in some way, expression of one or more particular genes. In some embodiments, such genes are apposed or “in the neighborhood” of a given regulatory element. In some embodiments, such genes are located quite far from a given regulatory element. In some embodiments, a regulatory element impairs or enhances transcription of one or more genes. In some embodiments, a regulatory element may be located in cis to a gene being regulated. In some embodiments, a regulatory element may be located in trans to a gene being regulated. For example, in some embodiments, a regulatory sequence refers to a nucleic acid sequence which is regulates expression of a gene product operably linked to a regulatory sequence. In some such embodiments, this sequence may be an enhancer sequence and other regulatory elements which regulate expression of a gene product.

Sample: As used herein, the term “sample” typically refers to an aliquot of material obtained or derived from a source of interest. In some embodiments, a source of interest is a biological or environmental source. In some embodiments, a source of interest may be or comprise a cell or an organism, such as a microbe (e.g., virus), a plant, or an animal (e.g., a human). In some embodiments, a source of interest is or comprises biological tissue or fluid. In some embodiments, a biological tissue or fluid may be or comprise amniotic fluid, aqueous humor, ascites, bile, bone marrow, blood, breast milk, cerebrospinal fluid, cerumen, chyle, chime, ejaculate, endolymph, exudate, feces, gastric acid, gastric juice, lymph, mucus, pericardial fluid, perilymph, peritoneal fluid, pleural fluid, pus, rheum, saliva, sebum, semen, serum, smegma, sputum, synovial fluid, sweat, tears, urine, vaginal secretions, vitreous humour, vomit, and/or combinations or component(s) thereof. In some embodiments, a biological fluid may be or comprise an intracellular fluid, an extracellular fluid, an intravascular fluid (blood plasma), an interstitial fluid, a lymphatic fluid, and/or a transcellular fluid. In some embodiments, a biological fluid may be or comprise a plant exudate. In some embodiments, a biological tissue or sample may be obtained, for example, by aspirate, biopsy (e.g., fine needle or tissue biopsy), swab (e.g., oral, nasal, skin, or vaginal swab), scraping, surgery, washing or lavage (e.g., bronchioalveolar, ductal, nasal, ocular, oral, uterine, vaginal, or other washing or lavage). In some embodiments, a biological sample is or comprises cells obtained from an individual. In some embodiments, a sample is a “primary sample” obtained directly from a source of interest by any appropriate means. In some embodiments, as will be clear from context, the term “sample” refers to a preparation that is obtained by processing (e.g., by removing one or more components of and/or by adding one or more agents to) a primary sample. For example, filtering using a semi-permeable membrane. Such a “processed sample” may comprise, for example nucleic acids or proteins extracted from a sample or obtained by subjecting a primary sample to one or more techniques such as amplification or reverse transcription of nucleic acid, isolation and/or purification of certain components, etc.

Selective expression: As used herein, the term “selective expression” or “selectively expresses” refers to expression of a coding sequence, gene, transgene, or polynucleotide (e.g., a therapeutic polynucleotide) of interest predominately in certain specific cell types (e.g., inner ear cells, e.g., inner ear supporting cells).

Subject: As used herein, the term “subject” refers an organism, typically a mammal (e.g., a human, in some embodiments including prenatal human forms). In some embodiments, a subject is suffering from a relevant disease, disorder or condition. In some embodiments, a subject is susceptible to a disease, disorder, or condition. In some embodiments, a subject displays one or more symptoms or characteristics of a disease, disorder or condition. In some embodiments, a subject does not display any symptom or characteristic of a disease, disorder, or condition. In some embodiments, a subject is someone with one or more features characteristic of susceptibility to or risk of a disease, disorder, or condition. In some embodiments, a subject is a patient. In some embodiments, a subject is an individual to whom diagnosis and/or therapy is and/or has been administered.

Substantially: As used herein, the term “substantially” refers to a qualitative condition of exhibiting total or near-total extent or degree of a characteristic or property of interest. One of ordinary skill in the art will understand that biological and chemical phenomena rarely, if ever, go to completion and/or proceed to completeness or achieve or avoid an absolute result. The term “substantially” is therefore used herein to capture a potential lack of completeness inherent in many biological and chemical phenomena.

Supporting cell: As used herein, the term “support cell,” “supporting cell,” “inner ear support cell,” or “inner ear supporting cell” refers to cells of the inner ear that maintain the structure of the inner ear and maintain the environment of the sensory epithelium of the inner ear. In some aspects, inner ear supporting cells include, but are not limited to, inner phalangeal cells/border cells (IPhC), inner pillar cells (IPC), outer pillar cells (OPC), Deiters' cells rows 1 and 2 (DC1/2), Deiters' cells row 3 (DC3), Hensen's cells (Hec), Claudius cells/outer sulcus cells (CC/OSC), interdental cells (Idc), inner sulcus cells (ISC), Kolliker's organ cells (KO), greater ridge epithelial ridge cells (GER) (including lateral greater epithelial ridge cells (LGER)), and OC90+ cells (OC90), fibroblasts, and other cells of the lateral wall.

Treatment: As used herein, the term “treatment” (also “treat” or “treating”) refers to any administration of a therapy that partially or completely alleviates, ameliorates, eliminates, reverses, relieves, inhibits, delays onset of, reduces severity of, and/or reduces incidence of one or more symptoms, features, and/or causes of a particular disease, disorder, and/or condition. In some embodiments, such treatment may be of a subject who does not exhibit signs of the relevant disease, disorder and/or condition and/or of a subject who exhibits only early signs of the disease, disorder, and/or condition. Alternatively, or additionally, such treatment may be of a subject who exhibits one or more established signs of the relevant disease, disorder and/or condition. In some embodiments, treatment may be of a subject who has been diagnosed as suffering from the relevant disease, disorder, and/or condition. In some embodiments, treatment may be of a subject known to have one or more susceptibility factors that are statistically correlated with increased risk of development of a given disease, disorder, and/or condition.

Variant: As used herein, the term “variant” refers to a version of something, e.g., a gene sequence, that is different, in some way, from another version. To determine if something is a variant, a reference version is typically chosen and a variant is different relative to that reference version. In some embodiments, a variant can have the same or a different (e.g., increased or decreased) level of activity or functionality than a wild type sequence. For example, in some embodiments, a variant can have improved functionality as compared to a wild-type sequence if it is, e.g., codon-optimized to resist degradation, e.g., by an inhibitory nucleic acid, e.g., miRNA. Such a variant is referred to herein as a gain-of-function variant. In some embodiments, a variant has a reduction or elimination in activity or functionality or a change in activity that results in a negative outcome (e.g., increased electrical activity resulting in chronic depolarization that leads to cell death). Such a variant is referred to herein as a loss-of-function variant. For example, in some embodiments, a GJB2 gene sequence is a wild-type sequence, which encodes a functional protein and exists in a majority of members of species with genomes containing the GJB2 gene. In some such embodiments, a gain-of-function variant can be a gene sequence of GJB2 that contains one or more nucleotide differences relative to a wild-type GJB2 gene sequence. In some embodiments, a gain-of-function variant is a codon-optimized sequence which encodes a transcript or polypeptide that may have improved properties (e.g., less susceptibility to degradation, e.g., less susceptibility to miRNA mediated degradation) than its corresponding wild type (e.g., non-codon optimized) version. In some embodiments, a loss-of-function variant has one or more changes that result in a transcript or polypeptide that is defective in some way (e.g., decreased function, non-functioning) relative to the wild type transcript and/or polypeptide. For example, in some embodiments, a mutation in a GJB2 sequence results in a non-functional or otherwise defective connexin 26 (Cx26) protein.

### DETAILED DESCRIPTION OF CERTAIN EMBODIMENTS

### Hearing Loss

Generally, an ear can be described as including: an outer ear, middle ear, inner ear, hearing (acoustic) nerve, and auditory system (which processes sound as it travels from the ear to the brain). In addition to detecting sound, ears also help to maintain balance. Thus, in some embodiments, disorders of the inner ear can cause hearing loss, tinnitus, vertigo, imbalance, or combinations thereof.

Hearing loss can be the result of genetic factors, environmental factors, or a combination of genetic and environmental factors. About half of all people who have tinnitus—phantom noises in their auditory system (ringing, buzzing, chirping, humming, or beating)—also have an over-sensitivity to/reduced tolerance for certain sound frequency and volume ranges, known as hyperacusis (also spelled hyperacousis). A variety of nonsyndromic and syndromic-related hearing losses will be known to those of skill in the art (e.g., DFNB1 and DFNA3. or Bart-Pumphrey syndrome, hystrix-like ichthyosis with deafness (HID), palmoplantar keratoderma with deafness, keratitis-ichthyosis-deafness (KID) syndrome and Vohwinkel syndrome, respectively). Environmental causes of hearing impairment or loss may include, e.g., certain medications, specific infections before or after birth, and/or exposure to loud noise over an extended period. In some embodiments, hearing loss can result from noise, ototoxic agents, presbycusis, disease, infection or cancers that affect specific parts of the ear. In some embodiments, ischemic damage can cause hearing loss via pathophysiological mechanisms. In some embodiments, intrinsic abnormalities, like congenital mutations to genes that play an important role in cochlear anatomy or physiology, or genetic or anatomical changes in supporting and/or hair cells can be responsible for or contribute to hearing loss.

Hearing loss and/or deafness is one of the most common human sensory deficits, and can occur for many reasons. In some embodiments, a subject may be born with hearing loss or without hearing, while others may lose hearing slowly over time. Approximately 36 million American adults report some degree of hearing loss, and one in three people older than 60 and half of those older than 85 experience hearing loss. Approximately 1.5 in 1,000 children are born with profound hearing loss, and another two to three per 1,000 children are born with partial hearing loss (Smith et al., 2005, Lancet 365:879-890, which is incorporated in its entirety herein by reference). More than half of these cases are attributed to a genetic basis (Di Domenico, et al., 2011, J. Cell. Physiol. 226:2494-2499, which is incorporated in its entirety herein by reference).

Treatments for hearing loss currently consist of hearing amplification for mild to severe losses and cochlear implantation for severe to profound losses (Kral and O'Donoghue, 2010, N. Engl. J. Med. 363:1438-1450, which is incorporated in its entirety herein by reference). Recent research in this arena has focused on cochlear hair cell regeneration, applicable to the most common forms of hearing loss, including presbycusis, noise damage, infection, and ototoxicity. There remains a need for effective treatments, such as gene therapy, which can repair and/or mitigate a source of a hearing problem (see e.g., WO 2018/039375, WO 2019/165292, and PCT filing application US2019/060328, each of which is incorporated in its entirety herein by reference).

In some embodiments, nonsyndromic hearing loss and/or deafness is not associated with other signs and symptoms. In some embodiments, syndromic hearing loss and/or deafness occurs in conjunction with abnormalities in other parts of the body. Approximately 70 percent to 80 percent of genetic hearing loss and/or deafness cases are nonsyndromic; remaining cases are often caused by specific genetic syndromes. Nonsyndromic deafness and/or hearing loss can have different patterns of inheritance, and can occur at any age. Types of nonsyndromic deafness and/or hearing loss are generally named according to their inheritance patterns. For example, autosomal dominant forms are designated DFNA, autosomal recessive forms are DFNB, and X-linked forms are DFN. Each type is also numbered in the order in which it was first described. For example, DFNA1 was the first described autosomal dominant type of nonsyndromic deafness. Between 75 percent and 80 percent of genetically causative hearing loss and/or deafness cases are inherited in an autosomal recessive pattern, which means both copies of the gene in each cell have mutations. Usually, each parent of an individual with autosomal recessive hearing loss and/or deafness is a carrier of one copy of the mutated gene, but is not affected by this form of hearing loss. Another 20 percent to 25 percent of nonsyndromic hearing loss and/or deafness cases are autosomal dominant, which means one copy of the altered gene in each cell is sufficient to result in deafness and/or hearing loss. People with autosomal dominant deafness and/or hearing loss most often inherit an altered copy of the gene from a parent who is deaf and/or has hearing loss. Between 1 to 2 percent of cases of deafness and/or hearing loss show an X-linked pattern of inheritance, which means the mutated gene responsible for the condition is located on the X chromosome (one of the two sex chromosomes). Males with X-linked nonsyndromic hearing loss and/or deafness tend to develop more severe hearing loss earlier in life than females who inherit a copy of the same gene mutation. A characteristic of X-linked inheritance is that fathers cannot pass X-linked traits to their sons. Mitochondrial nonsyndromic deafness, which results from changes to mitochondrial DNA, occurs in less than one percent of cases in the United States. The altered mitochondrial DNA is passed from a mother to all of her sons and daughters. This type of deafness is not inherited from fathers. The causes of syndromic and nonsyndromic deafness and/or hearing loss are complex. Researchers have identified more than 30 genes that, when altered, are associated with syndromic and/or nonsyndromic deafness and/or hearing loss; however, some of these genes have not been fully characterized. Different mutations in the same gene can be associated with different types of deafness and/or hearing loss, and some genes are associated with both syndromic and nonsyndromic deafness and/or hearing loss.

In some embodiments, deafness and/or hearing loss can be conductive (arising from the ear canal or middle ear), sensorineural (arising from the inner ear or auditory nerve), or mixed. In some embodiments, nonsyndromic deafness and/or hearing loss is associated with permanent hearing loss caused by damage to structures in the inner ear (sensorineural deafness). In some embodiments, sensorineural hearing loss can be due to poor hair cell function. In some embodiments, sensorineural hearing impairments involve the eighth cranial nerve (the vestibulocochlear nerve) or the auditory portions of the brain. In some such embodiments, only the auditory centers of the brain are affected. In such a situation, cortical deafness may occur, where sounds may be heard at normal thresholds, but quality of sound perceived is so poor that speech cannot be understood. Hearing loss that results from changes in the middle ear is called conductive hearing loss. Some forms of nonsyndromic deafness and/or hearing loss involve changes in both the inner ear and the middle ear, called mixed hearing loss. Hearing loss and/or deafness that is present before a child learns to speak can be classified as prelingual or congenital. Hearing loss and/or deafness that occurs after the development of speech can be classified as postlingual. Most autosomal recessive loci related to syndromic or nonsyndromic hearing loss cause prelingual severe-to-profound hearing loss.

As is known to those of skill in the art, hair cells are sensory receptors for both auditory and vestibular systems of vertebrate ears. Hair cells detect movement in the environment and, in mammals, hair cells are located within the cochlea of the ear, in the organ of Corti. Mammalian ears are known to have two types of hair cells—inner hair cells and outer hair cells. Outer hair cells can amplify low level sound frequencies, either through mechanical movement of hair cell bundles or electrically-driven movement of hair cell soma. Inner hair cells transform vibrations in cochlear fluid into electrical signals that the auditory nerve transmits to the brain. In some embodiments, hair cells may be abnormal at birth, or damaged during the lifetime of an individual. In some embodiments, outer hair cells may be able to regenerate. In some embodiments, inner hair cells are not capable of regeneration after illness or injury. In some embodiments, sensorineural hearing loss is due to abnormalities in hair cells.

As is known to those of skill in the art, hair cells do not occur in isolation, and their function is supported by a wide variety of cells which can collectively be referred to as supporting cells. Supporting cells may fulfil numerous functions, and include a number of cell types, including but not limited to Hensen's cells, Deiters' cells, pillar cells, Claudius cells, inner phalangeal cells, and border cells. In some embodiments, sensorineural hearing loss is due to abnormalities in supporting cells. In some embodiments, supporting cells may be abnormal at birth, or damaged during the lifetime of an individual. In some embodiments, supporting cells may be able to regenerate. In some embodiments, certain supporting cells may not be capable of regeneration.

### Gap Junction Beta-2 (GJB2)

The GJB2 gene is highly conserved across the mammalian class and encodes connexin 26 (Cx26) (also referred to as gap junction beta-2 (GJB2) protein). Connexin 26 is a member of the gap junction protein family, which is also known as the connexin family. Gap junction proteins are specialized proteins, involved in intracellular communication. Mutations in the human GJB2 gene have been associated with hearing loss and deafness (Amorini et al., Ann. Hum. Genet. 79(5):341-349, 2015; Qing et al., Genet. Test Mol. Biomarkers 19(1):52-58, 2015).

The human GJB2 gene is located on chromosome 13q12. It contains two transcriptional isoforms beginning from alternative transcriptional start sites, both of which contain two exons and a single intron encompassing a total of about 5 kilobases (kb) (approximately 5,469 or 4,675 nucleotides respectively) (NCBI Gene ID 2706, NCBI Reference Sequence: NG_008358.1). Both human GJB2 mRNA isoforms comprise a second exon, which completely encodes a full-length connexin 26 in exon two. This coding sequence is approximately 681 nucleotides, and encodes a connexin 26 that is 226 amino acids in length.

A monomer of connexin 26 includes four transmembrane helices linked by two extracellular loops and one shorter intracellular loop, with N- and C-termini on the cytosolic side of the plasma membrane. Gap junctions between cells can be formed in a homomeric and/or heteromeric manner. Connexin 26 has been shown to form functional homomeric channels, as well as functional heteromeric channels with at least connexin 30, connexin 32, connexin 46, and connexin 50. In some embodiments, GJB2 gene associated sensorineural hearing loss (e.g., nonsyndromic or syndromic) may be due to compound heterozygous mutations in GJB2 and in an alternative connexin protein encoding gene. The gap junctions created with connexin 26 transport potassium ions and certain other small molecules across cells. Connexin 26 helps maintain the correct level of intracellular potassium ions, and is required for the maturation of certain cells in the cochlea.

A human GJB2 gene is expressed in a number of tissues, but is known to be involved in important cellular homeostasis functions in the epidermis and inner ear. Within the inner ear, connexin 26 is synthesized by all supporting cell types within the organ of corti, including the inner and outer pillar cells, root cells, interdental cells, fibrocytes from the underlying connective tissue, and basal and intermediate cells from the stria vascularis. In addition, connexin 26 is known to be present in mesenchymal cells in the lateral wall, and type 1 neurons in the spiral ganglion.

The human GJB2 gene has a defined 128 bp long basal promoter just upstream of the canonical first exon in the most abundant isoform. This sequence includes a TATA box and two GC boxes, which are known to be bound by the Sp1 and Sp3 TFs.

There are over 200 defined mutations of GJB2, which show some level of pathogenicity, and various mutations in the GJB2 gene have been associated with hearing loss (e.g., non-syndromic sensorineural hearing loss or syndromic sensorineural hearing loss). For example, the c.35delG allele was found on 65.5% of patients from Eastern Sicily (Amorini et al., Ann. Hum. Genet. 79(5):341-349, 2015). Additional exemplary mutations in a GJB2 gene detected in subjects having nonsyndromic sensorineural hearing loss or syndromic sensorineural hearing loss, and methods of sequencing a nucleic acid encoding GJB2 are described in, e.g., Snoeckx et al., Am. J. Hum. Genet 77: 945-957, 2005; Welch et al., Am. J. Med. Genet A 143: 1567-1573, 2007; Zelante et al., Hum. Mol. Genet. 6:1605-1609, 1997; and Tsukada et al., Annals of Otology, Rhinology & Laryngology. 2015, Vol. 124(5S) 61S-76S, each of which is incorporated in its entirety herein by reference. Methods of detecting mutations in a gene are well-known in the art. Non-limiting examples of such techniques include: real-time polymerase chain reaction (RT-PCR), PCR, Sanger sequencing, Next-generation sequencing, Southern blotting, and Northern blotting.

Multiple disease states associated with sensorineural hearing loss with either non-syndromic or syndromic manifestations have been linked with specific mutations of the human GJB2 gene (see Nickel & Forge, Curr Opin Otolaryngol Head Neck Surg. 2008 October; 16(5):452-7, which is incorporated in its entirety herein by reference). Human GBJ2 gene mutations which lead to syndromic or nonsyndromic hearing loss vary from large deletions that remove either the entirety of GJB2 or GJB2 gene regulatory regions, to hundreds of small scale alterations including nonsense, missense, indels (leading to phase shifting), and splice-site point mutations.

In some embodiments, GJB2 gene mutations such as Gly59Ser, and Asn52Lys are associated with Bart-Pumphrey syndrome. A syndrome defined by manifestations of thickened skin, wart-like growths, and generally congenital moderate to profound sensorineural hearing loss. In other embodiments, GJB2 gene mutations such as Aspn50Asn are associated with Hystrix-like Ichthyosis with deafness & Keratitis-ichthyosis-deafness syndrome. These syndromes are associated with dry scaly skin, generally congenital profound sensorineural hearing loss, and in Keratitis-ichthyosis-deafness syndrome, additional inflammation of the cornea.

In some embodiments, GJB2 gene missense mutations are associated with Palmoplantar keratoderma with deafness. A syndrome associated with thick skin on the palms of the hands and soles of the feet, and mild to profound sensorineural hearing loss which begins in early childhood and gets worse over time, affected individuals may have particular trouble hearing high-pitched sounds. While in other embodiments, GJB2 gene missense mutations are associated with Vohwinkel syndrome. A syndrome associated with skin abnormalities (e.g., thick bands of fibrous tissue around their fingers and toes that may cut off the circulation to the digits and result in spontaneous amputation) and sensorineural hearing loss.

In some embodiments, GJB2 gene mutations are associated with nonsyndromic hearing loss, which may be inherited in either a dominant (e.g., DFNA3) or recessive manner (DFNB1). In some embodiments, loss of function GJB2 gene mutations are associated with nonsyndromic DFNB1 which is inherited in an autosomal recessive manner and presents as mild to profound hearing loss that is generally prelingual and does not become more severe over time. It is estimated that DFNB1 is present in approximately 14 out of every 100,000 live births in the US and EU5. It has been postulated that an early but not always congenital onset of DFNB1 hearing impairment could be followed by a quick progression of the hearing loss. In general, DFNB1 patents treatment options include education, hearing aids, and cochlear implants. Patients generally do not have additional symptoms, and live a normal lifespan. It is estimated that DFNB1 accounts for about 50% of congenital severe-to-profound autosomal recessive non-syndromic hearing loss in many first world countries (e.g., US, France, Britain, and Australia).

In some embodiments, sensorineural hearing loss due to GJB2 gene mutations are inherited in an autosomal dominant manner as nonsyndromic DFNA3. These mutations are generally dominant negative missense mutations that prevent the formation of necessary functional gap junctions. This disease state presents with hearing loss that can be either prelingual or postlingual, ranging from mild to profound, which generally becomes more severe over time.

### GJB2 Polynucleotides

Among other things, the present disclosure provides polynucleotides, e.g., polynucleotides comprising a GJB2 gene or characteristic portion thereof, as well as compositions including such polynucleotides and methods utilizing such polynucleotides and/or compositions.

In some embodiments, a polynucleotide comprising a GJB2 gene or characteristic portion thereof can be DNA or RNA. In some embodiments, DNA can be genomic DNA or cDNA. In some embodiments, RNA can be an mRNA. In some embodiments, a polynucleotide comprises exons and/or introns of a GJB2 gene.

In some embodiments, a gene product is expressed from a polynucleotide comprising a GJB2 gene or characteristic portion thereof. In some embodiments, expression of such a polynucleotide can utilize one or more control elements (e.g., promoters, enhancers, splice sites, poly-adenylation sites, translation initiation sites, etc.). Thus, in some embodiments, a polynucleotide provided herein can include one or more control elements.

In some embodiments, a GJB2 gene is a mammalian GJB2 gene. In some embodiments, a GJB2 gene is a murine GJB2 gene. In some embodiments, a GJB2 gene is a primate GJB2 gene. In some embodiments, a GJB2 gene is a human GJB2 gene. An exemplary human GJB2 coding cDNA sequence is or includes the sequence of SEQ ID NO: 1 or SEQ ID NO: 2. An exemplary human GJB2 spliced cDNA sequence with untranslated regions is or includes the sequence of SEQ ID NO: 3. An alternative transcriptional start site exemplary human GJB2 spliced cDNA sequence with untranslated regions is or includes the sequence of SEQ ID NO: 4. An exemplary human GJB2 genomic DNA sequence can be found in SEQ ID NO: 5.

Exemplary Human GJB2 cDNA coding Sequence
(SEQ ID NO: 1)
ATGGATTGGGGCACGCTGCAGACGATCCTGGGGGGTGTGAACAAACACTCCACCAGCATTGGAAA
GATCTGGCTCACCGTCCTCTTCATTTTTCGCATTATGATCCTCGTTGTGGCTGCAAAGGAGGTGT
GGGGAGATGAGCAGGCCGACTTTGTCTGCAACACCCTGCAGCCAGGCTGCAAGAACGTGTGCTAC
GATCACTACTTCCCCATCTCCCACATCCGGCTATGGGCCCTGCAGCTGATCTTCGTGTCCACGCC
AGCGCTCCTAGTGGCCATGCACGTGGCCTACCGGAGACATGAGAAGAAGAGGAAGTTCATCAAGG
GGGAGATAAAGAGTGAATTTAAGGACATCGAGGAGATCAAAACCCAGAAGGTCCGCATCGAAGGC
TCCCTGTGGTGGACCTACACAAGCAGCATCTTCTTCCGGGTCATCTTCGAAGCCGCCTTCATGTA
CGTCTTCTATGTCATGTACGACGGCTTCTCCATGCAGCGGCTGGTGAAGTGCAACGCCTGGCCTT
GTCCCAACACTGTGGACTGCTTTGTGTCCCGGCCCACGGAGAAGACTGTCTTCACAGTGTTCATG
ATTGCAGTGTCTGGAATTTGCATCCTGCTGAATGTCACTGAATTGTGTTATTTGCTAATTAGATA
TTGTTCTGGGAAGTCAAAAAAGCCAGTT
Exemplary Human GJB2 cDNA coding Sequence
(SEQ ID NO: 2)
ATGGATTGGGGCACGCTGCAGACGATCCTGGGGGGTGTGAACAAACACTCCACCAGCATTGGAAA
GATCTGGCTCACCGTCCTCTTCATTTTTCGCATTATGATCCTCGTTGTGGCTGCAAAGGAGGTGT
GGGGAGATGAGCAGGCCGACTTTGTCTGCAACACCCTGCAGCCAGGCTGCAAGAACGTGTGCTAC
GATCACTACTTCCCCATCTCCCACATCCGGCTATGGGCCCTGCAGCTGATCTTCGTGTCCACGCC
AGCGCTCCTAGTGGCCATGCACGTGGCCTACCGGAGACATGAGAAGAAGAGGAAGTTCATCAAGG
GGGAGATAAAGAGTGAATTTAAGGACATCGAGGAGATCAAAACCCAGAAGGTCCGCATCGAAGGC
TCCCTGTGGTGGACCTACACAAGCAGCATCTTCTTCCGGGTCATCTTCGAAGCCGCCTTCATGTA
CGTCTTCTATGTCATGTACGACGGCTTCTCCATGCAGCGGCTGGTGAAGTGCAACGCCTGGCCTT
GTCCCAACACTGTGGACTGCTTTGTGTCCCGGCCCACGGAGAAGACTGTCTTCACAGTGTTCATG
ATTGCAGTGTCTGGAATTTGCATCCTGCTGAATGTCACTGAATTGTGTTATTTGCTAATTAGATA
TTGTTCTGGGAAGTCAAAAAAGCCAGTTTAA
Exemplary spliced Human GJB2 isoform 1 cDNA including untranslated regions Sequence
(SEQ ID NO: 3)
GTTGCGGCCCCGCAGCGCCCGCGCGCTCCTCTCCCCGACTCGGAGCCCCTCGGCGGCGCCCGGCC
CAGGACCCGCCTAGGAGCGCAGGAGCCCCAGCGCAGAGACCCCAACGCCGAGACCCCCGCCCCGG
CCCCGCCGCGCTTCCTCCCGACGCAGAGCAAACCGCCCAGAGTAGAAGATGGATTGGGGCACGCT
GCAGACGATCCTGGGGGGTGTGAACAAACACTCCACCAGCATTGGAAAGATCTGGCTCACCGTCC
TCTTCATTTTTCGCATTATGATCCTCGTTGTGGCTGCAAAGGAGGTGTGGGGAGATGAGCAGGCC
GACTTTGTCTGCAACACCCTGCAGCCAGGCTGCAAGAACGTGTGCTACGATCACTACTTCCCCAT
CTCCCACATCCGGCTATGGGCCCTGCAGCTGATCTTCGTGTCCACGCCAGCGCTCCTAGTGGCCA
TGCACGTGGCCTACCGGAGACATGAGAAGAAGAGGAAGTTCATCAAGGGGGAGATAAAGAGTGAA
TTTAAGGACATCGAGGAGATCAAAACCCAGAAGGTCCGCATCGAAGGCTCCCTGTGGTGGACCTA
CACAAGCAGCATCTTCTTCCGGGTCATCTTCGAAGCCGCCTTCATGTACGTCTTCTATGTCATGT
ACGACGGCTTCTCCATGCAGCGGCTGGTGAAGTGCAACGCCTGGCCTTGTCCCAACACTGTGGAC
TGCTTTGTGTCCCGGCCCACGGAGAAGACTGTCTTCACAGTGTTCATGATTGCAGTGTCTGGAAT
TTGCATCCTGCTGAATGTCACTGAATTGTGTTATTTGCTAATTAGATATTGTTCTGGGAAGTCAA
AAAAGCCAGTTTAACGCATTGCCCAGTTGTTAGATTAAGAAATAGACAGCATGAGAGGGATGAGG
CAACCCGTGCTCAGCTGTCAAGGCTCAGTCGCTAGCATTTCCCAACACAAAGATTCTGACCTTAA
ATGCAACCATTTGAAACCCCTGTAGGCCTCAGGTGAAACTCCAGATGCCACAATGGAGCTCTGCT
CCCCTAAAGCCTCAAAACAAAGGCCTAATTCTATGCCTGTCTTAATTTTCTTTCACTTAAGTTAG
TTCCACTGAGACCCCAGGCTGTTAGGGGTTATTGGTGTAAGGTACTTTCATATTTTAAACAGAGG
ATATCGGCATTTGTTTCTTTCTCTGAGGACAAGAGAAAAAAGCCAGGTTCCACAGAGGACACAGA
GAAGGTTTGGGTGTCCTCCTGGGGTTCTTTTTGCCAACTTTCCCCACGTTAAAGGTGAACATTGG
TTCTTTCATTTGCTTTGGAAGTTTTAATCTCTAACAGTGGACAAAGTTACCAGTGCCTTAAACTC
TGTTACACTTTTTGGAAGTGAAAACTTTGTAGTATGATAGGTTATTTTGATGTAAAGATGTTCTG
GATACCATTATATGTTCCCCCTGTTTCAGAGGCTCAGATTGTAATATGTAAATGGTATGTCATTC
GCTACTATGATTTAATTTGAAATATGGTCTTTTGGTTATGAATACTTTGCAGCACAGCTGAGAGG
CTGTCTGTTGTATTCATTGTGGTCATAGCACCTAACAACATTGTAGCCTCAATCGAGTGAGACAG
ACTAGAAGTTCCTAGTGATGGCTTATGATAGCAAATGGCCTCATGTCAAATATTTAGATGTAATT
TTGTGTAAGAAATACAGACTGGATGTACCACCAACTACTACCTGTAATGACAGGCCTGTCCAACA
CATCTCCCTTTTCCATGACTGTGGTAGCCAGCATCGGAAAGAACGCTGATTTAAAGAGGTCGCTT
GGGAATTTTATTGACACAGTACCATTTAATGGGGAGGACAAAATGGGGCAGGGGAGGGAGAAGTT
TCTGTCGTTAAAAACAGATTTGGAAAGACTGGACTCTAAAGTCTGTTGATTAAAGATGAGCTTTG
TCTACTTCAAAAGTTTGTTTGCTTACCCCTTCAGCCTCCAATTTTTTAAGTGAAAATATAGCTAA
TAACATGTGAAAAGAATAGAAGCTAAGGTTTAGATAAATATTGAGCAGATCTATAGGAAGATTGA
ACCTGAATATTGCCATTATGCTTGACATGGTTTCCAAAAAATGGTACTCCACATATTTCAGTGAG
GGTAAGTATTTTCCTGTTGTCAAGAATAGCATTGTAAAAGCATTTTGTAATAATAAAGAATAGCT
TTAATGATATGCTTGTAACTAAAATAATTTTGTAATGTATCAAATACATTTAAAACATTAAAATA
TAATCTCTATAATAA
Exemplary spliced Human GJB2 isoform X1 cDNA including untranslated regions
Sequence
(SEQ ID NO: 4)
TTTAGGACCCTTGTTCGCGAAGAGGTGGTGTGCGGCTGAGACCCGCGTCCTCAGGACGGTTCCAT
CAGTGCCTCGATCCTGCCCCACTGGAGGAGGAAGGCAGCCCGAACAGCGCTCACCTAACTAACAG
CTGCTGAGAGCTGGGTTCCGTGGCCATGCACCTGGGACTGCCTTGAGAAGCGTGAGCAAACCGCC
CAGAGTAGAAGATGGATTGGGGCACGCTGCAGACGATCCTGGGGGGTGTGAACAAACACTCCACC
AGCATTGGAAAGATCTGGCTCACCGTCCTCTTCATTTTTCGCATTATGATCCTCGTTGTGGCTGC
AAAGGAGGTGTGGGGAGATGAGCAGGCCGACTTTGTCTGCAACACCCTGCAGCCAGGCTGCAAGA
ACGTGTGCTACGATCACTACTTCCCCATCTCCCACATCCGGCTATGGGCCCTGCAGCTGATCTTC
GTGTCCACGCCAGCGCTCCTAGTGGCCATGCACGTGGCCTACCGGAGACATGAGAAGAAGAGGAA
GTTCATCAAGGGGGAGATAAAGAGTGAATTTAAGGACATCGAGGAGATCAAAACCCAGAAGGTCC
GCATCGAAGGCTCCCTGTGGTGGACCTACACAAGCAGCATCTTCTTCCGGGTCATCTTCGAAGCC
GCCTTCATGTACGTCTTCTATGTCATGTACGACGGCTTCTCCATGCAGCGGCTGGTGAAGTGCAA
CGCCTGGCCTTGTCCCAACACTGTGGACTGCTTTGTGTCCCGGCCCACGGAGAAGACTGTCTTCA
CAGTGTTCATGATTGCAGTGTCTGGAATTTGCATCCTGCTGAATGTCACTGAATTGTGTTATTTG
CTAATTAGATATTGTTCTGGGAAGTCAAAAAAGCCAGTTTAACGCATTGCCCAGTTGTTAGATTA
AGAAATAGACAGCATGAGAGGGATGAGGCAACCCGTGCTCAGCTGTCAAGGCTCAGTCGCTAGCA
TTTCCCAACACAAAGATTCTGACCTTAAATGCAACCATTTGAAACCCCTGTAGGCCTCAGGTGAA
ACTCCAGATGCCACAATGGAGCTCTGCTCCCCTAAAGCCTCAAAACAAAGGCCTAATTCTATGCC
TGTCTTAATTTTCTTTCACTTAAGTTAGTTCCACTGAGACCCCAGGCTGTTAGGGGTTATTGGTG
TAAGGTACTTTCATATTTTAAACAGAGGATATCGGCATTTGTTTCTTTCTCTGAGGACAAGAGAA
AAAAGCCAGGTTCCACAGAGGACACAGAGAAGGTTTGGGTGTCCTCCTGGGGTTCTTTTTGCCAA
CTTTCCCCACGTTAAAGGTGAACATTGGTTCTTTCATTTGCTTTGGAAGTTTTAATCTCTAACAG
TGGACAAAGTTACCAGTGCCTTAAACTCTGTTACACTTTTTGGAAGTGAAAACTTTGTAGTATGA
TAGGTTATTTTGATGTAAAGATGTTCTGGATACCATTATATGTTCCCCCTGTTTCAGAGGCTCAG
ATTGTAATATGTAAATGGTATGTCATTCGCTACTATGATTTAATTTGAAATATGGTCTTTTGGTT
ATGAATACTTTGCAGCACAGCTGAGAGGCTGTCTGTTGTATTCATTGTGGTCATAGCACCTAACA
ACATTGTAGCCTCAATCGAGTGAGACAGACTAGAAGTTCCTAGTGATGGCTTATGATAGCAAATG
GCCTCATGTCAAATATTTAGATGTAATTTTGTGTAAGAAATACAGACTGGATGTACCACCAACTA
CTACCTGTAATGACAGGCCTGTCCAACACATCTCCCTTTTCCATGACTGTGGTAGCCAGCATCGG
AAAGAACGCTGATTTAAAGAGGTCGCTTGGGAATTTTATTGACACAGTACCATTTAATGGGGAGG
ACAAAATGGGGCAGGGGAGGGAGAAGTTTCTGTCGTTAAAAACAGATTTGGAAAGACTGGACTCT
AAAGTCTGTTGATTAAAGATGAGCTTTGTCTACTTCAAAAGTTTGTTTGCTTACCCCTTCAGCCT
CCAATTTTTTAAGTGAAAATATAGCTAATAACATGTGAAAAGAATAGAAGCTAAGGTTTAGATAA
ATATTGAGCAGATCTATAGGAAGATTGAACCTGAATATTGCCATTATGCTTGACATGGTTTCCAA
AAAATGGTACTCCACATATTTCAGTGAGGGTAAGTATTTTCCTGTTGTCAAGAATAGCATTGTAA
AAGCATTTTGTAATAATAAAGAATAGCTTTAATGATATGCTTGTAACTAAAATAATTTTGTAATG
TATCAAATACATTTAAAACATTAAAATATAATCTCTATAATAA
Exemplary Human GJB2 Genomic DNA Sequence
(SEQ ID NO: 5)
GTTGCGGCCCCGCAGCGCCCGCGCGCTCCTCTCCCCGACTCGGAGCCCCTCGGCGGCGCCCGGCC
CAGGACCCGCCTAGGAGCGCAGGAGCCCCAGCGCAGAGACCCCAACGCCGAGACCCCCGCCCCGG
CCCCGCCGCGCTTCCTCCCGACGCAGGTGAGCCCGCCGGCCCCGGACTGCCCGGCCAGGAACCTG
GCGCGGGGAGGGACCGCGAGACCCAGAGCGGTTGCCCGGCCGCGTGGGTCTCGGGGAACCGGGGG
GCTGGACCAACACACGTCCTTGGGCCGGGGGGCGGGGGCCGCCTTCTGGAGCGGGCGTTTCTGCG
GCCGAGCTCCGGAGCTGGAATGGGGCGGCCGGGGAAGTGGACGCGATGGCACCGCCCGGGGTGCG
AGTGGGGCCGGGCGCGCGCGGGAGGGGAAAAAGGCGCGGGCGAGCCGCCAGCGCGAGGTTTGTGG
TGTCGCCGATGTCCCTTCGGGGTACTCTAGCGCAGCCGCCTGGCTACTTGACCCACTGCCACCAA
ACGTTTTAAATTCACCGAAAGCTTAGCTTCGAAGCAAAGCTCCGTTTCGCCGGTGAAGCAGGAAG
CCTTCGCTGCAGGAACTGACCTTTACCTCTTGGAGCGGCTTCTGCAGAAAAATCCCCGGGCAGAG
ATTTGGGCGGAGTTTGCCTAGAACTAACGCGGAGCCAGCCGATCCCGGCCTACCCCGGGGCCAAG
ATTTCAGTGGCTTCCCTTTTTCCTAAACACTTCACGAGGGTCTGTTTCCGGGCTGTGCTCCCCGC
CTAGAAGGAAAATTTTTAGGACCCTTGTTCGCGAAGAGGTGGTGTGCGGCTGAGACCCGCGTCCT
CAGGACGGTTCCATCAGTGCCTCGATCCTGCCCCACTGGAGGAGGAAGGCAGCCCGAACAGCGCT
CACCTAACTAACAGCTGCTGAGAGCTGGGTTCCGTGGCCATGCACCTGGGACTGCCTTGAGAAGC
GTGGTACGGCCGTGTCCCCATGTGACCTTAGAGTCCCTTTCGAAACTGCTGTGCACAGTCGGTCA
CAATTTCAGACACTGGTGAGAAGGGTGGAGGAACCCTCTGGGGACAGCCAGGCAAGGTCGACCAC
CCATCACCTAAGGGTGGAGAAATTTAAGGGGTGAAGAGTCCCTTTTGCCTTTTCTGGATCCTGGT
GATTCACCTAGTGTCTTCCCTAAGGAACTGAACCAACTCCTCCGCTGGCCTCTGGCAGCCCTCCA
GGCGGTGCAGGATGGCGTGGGCCCGGTAGGAAGCTGCATGTAACCGCCCAGGGTCGGGAGGCCAG
GAGGGCAGCTCCTCCTCTGACTTGAATATTGAAAACAACTTCGTCCTGCTTCTGAGCCCCTCTTA
ACCCATGACCCCCTAGCCCATTGGGGAGTAAATCTTAATTTACTCCTCTTCCTGAAAAAGGATCT
TTAAAACAGGTAGCTTCAACTCAAGCTTTATAAAATAACAATATAGGGTTTCTCGGAACTGTATT
TTTCTCAGCTGATGGTAACTGGACAGGTCTGTAGAAGGGTGTATGACCTGGGTTTGGCAGGTGGA
AGAGGGCAAAGGATAAACCCCTCCTCCTGCAGCCCCATATTCTTGGCCAGGTGTATTGTTGTAAA
CCAGGAGAGAGTTTACTTCGGGGAGTATCCTGTTTTCCACTCAGTGAGGGCCAATGAAGAATGTC
TAATTCCATAAGATGCTTTTGTTAAAATCGGAATGTTGCTGTCCTCGGTGGTTCTGCTGTTGGGA
CGGGACTGGCCTGAGCTGTGGGTGCTGTAGCAGGACAACCAGCTCACCTAAGGGCCTCCCAGTCT
GGATTATCAATGGGTCAGTGCTGAACCTGGGCTAAAATATTGTTTTTTCCAATGATGTTGTCTTT
CCCAAGCTCAGTGAAGCTAAATGTTTCACAGGCCTATGTCAATCTGATGTAACTTTCGTGGCCAC
CTCTCTCCTGTTAGCCTCTGACCAAGGTGGCACTGGATGGTTTCTGCCTGACCTTGGTGCCCCGT
GGCAGCGACTGTGGGTCATGAAAGACATTCACTACGAGCCTGCTTCTGGAGTCCATCAGAAAACG
GGATGCAACTTGCCTAAAATGAGGAGAGGAGGATGCTTTTAAGAAAAAGAAGAAGGAGGATTCAC
TACCAGCTCTGAAGGGTGGAAAAGAGATGATTCATCCGGATTGTGGAGAGGGTGGAATCTTGTTT
AGGAGAGCGTTGGTTGTGGCAGGCAGGGTGTAACTATGAATCAGTGAAGACAATTCACATCCTGG
GATGAAAAGAAGGCCATGGGCTCACAGGAGATTATCCACTGGCCTCTCCACATCCGCTTGCAGTA
AGGAGTGTGGGACTCTCCCAAGCTTCAGCGCTGAACTGCAATGCAGTGACGTCGCTTAGCTGGGC
CAGTAACCGAGGGAGTTGAATTTTCTGTCATTTTAAAATAATGTGTCTTTTAAGAAACACTTTGA
AATTAAAACCACAGCCCACAATTATAATGCACTGTTGCAGCACTTATCAAAACAGATATGCTAAC
TGAGCCATCAGTGCCAGCCTGACAGTGAGGCCACCAAGCCATCCACAAAGCCTACACGAAAGTCT
GTGCTCACAGTGGCTTTTCTCCATGAAGAGGGCATTCCTAACCTCTTCCTTTCACGTAGGAGGAA
GCAAGGTCCTTTGTAAAATTTTAACTCGGGGTGCCTCAAATGTAAACTTAACCACTGGTAACAAC
AGTTTCACTGCTACATGCCACGTCTGTGAAAATTCATTCAAGACATTAAGGAAAGTGGCTCAGCA
GAGAGACTAGACATCTTATCCTCACGGTTCTCCTGTACTTGGCCTCTCAGCCTTTGAGCAAGGTT
GGCCCAAGCTAGTATCGGCCCCAGTGGTACAGCCAAAACTTGAGACTGCAAATGGATGCAGCTGT
TGAACGCTGAGTAACTTCTGCAGAGTCAGGAAGACCCAAGGAAGCTCTGCAGAGGATGCAGGGGT
ACGGTCAGAACCCCTGAGTGCCTTTCAGCTAACGAGGACTTTATGACACTCCCCAGCACAGCAAA
TTTTTATGATGTGTTTAAAGATTGGGTGAATTACTCAGGTGAACAAGCTACTTTTTATCAGAGAA
CACCTAAAAACACGTTCAAGAGGGTTTGGGAACTATACATTTAATCCTATGACAAACTAAGTTGG
TTCTGTCTTCACCTGTTTTGGTGAGGTTGTGTAAGAGTTGGTGTTTGCTCAGGAAGAGATTTAAG
CATGCTTGCTTACCCAGACTCAGAGAAGTCTCCCTGTTCTGTCCTAGCTAGTGATTCCTGTGTTG
TGTGCATTCGTCTTTTCCAGAGCAAACCGCCCAGAGTAGAAGATGGATTGGGGCACGCTGCAGAC
GATCCTGGGGGGTGTGAACAAACACTCCACCAGCATTGGAAAGATCTGGCTCACCGTCCTCTTCA
TTTTTCGCATTATGATCCTCGTTGTGGCTGCAAAGGAGGTGTGGGGAGATGAGCAGGCCGACTTT
GTCTGCAACACCCTGCAGCCAGGCTGCAAGAACGTGTGCTACGATCACTACTTCCCCATCTCCCA
CATCCGGCTATGGGCCCTGCAGCTGATCTTCGTGTCCACGCCAGCGCTCCTAGTGGCCATGCACG
TGGCCTACCGGAGACATGAGAAGAAGAGGAAGTTCATCAAGGGGGAGATAAAGAGTGAATTTAAG
GACATCGAGGAGATCAAAACCCAGAAGGTCCGCATCGAAGGCTCCCTGTGGTGGACCTACACAAG
CAGCATCTTCTTCCGGGTCATCTTCGAAGCCGCCTTCATGTACGTCTTCTATGTCATGTACGACG
GCTTCTCCATGCAGCGGCTGGTGAAGTGCAACGCCTGGCCTTGTCCCAACACTGTGGACTGCTTT
GTGTCCCGGCCCACGGAGAAGACTGTCTTCACAGTGTTCATGATTGCAGTGTCTGGAATTTGCAT
CCTGCTGAATGTCACTGAATTGTGTTATTTGCTAATTAGATATTGTTCTGGGAAGTCAAAAAAGC
CAGTTTAACGCATTGCCCAGTTGTTAGATTAAGAAATAGACAGCATGAGAGGGATGAGGCAACCC
GTGCTCAGCTGTCAAGGCTCAGTCGCTAGCATTTCCCAACACAAAGATTCTGACCTTAAATGCAA
CCATTTGAAACCCCTGTAGGCCTCAGGTGAAACTCCAGATGCCACAATGGAGCTCTGCTCCCCTA
AAGCCTCAAAACAAAGGCCTAATTCTATGCCTGTCTTAATTTTCTTTCACTTAAGTTAGTTCCAC
TGAGACCCCAGGCTGTTAGGGGTTATTGGTGTAAGGTACTTTCATATTTTAAACAGAGGATATCG
GCATTTGTTTCTTTCTCTGAGGACAAGAGAAAAAAGCCAGGTTCCACAGAGGACACAGAGAAGGT
TTGGGTGTCCTCCTGGGGTTCTTTTTGCCAACTTTCCCCACGTTAAAGGTGAACATTGGTTCTTT
CATTTGCTTTGGAAGTTTTAATCTCTAACAGTGGACAAAGTTACCAGTGCCTTAAACTCTGTTAC
ACTTTTTGGAAGTGAAAACTTTGTAGTATGATAGGTTATTTTGATGTAAAGATGTTCTGGATACC
ATTATATGTTCCCCCTGTTTCAGAGGCTCAGATTGTAATATGTAAATGGTATGTCATTCGCTACT
ATGATTTAATTTGAAATATGGTCTTTTGGTTATGAATACTTTGCAGCACAGCTGAGAGGCTGTCT
GTTGTATTCATTGTGGTCATAGCACCTAACAACATTGTAGCCTCAATCGAGTGAGACAGACTAGA
AGTTCCTAGTGATGGCTTATGATAGCAAATGGCCTCATGTCAAATATTTAGATGTAATTTTGTGT
AAGAAATACAGACTGGATGTACCACCAACTACTACCTGTAATGACAGGCCTGTCCAACACATCTC
CCTTTTCCATGACTGTGGTAGCCAGCATCGGAAAGAACGCTGATTTAAAGAGGTCGCTTGGGAAT
TTTATTGACACAGTACCATTTAATGGGGAGGACAAAATGGGGCAGGGGAGGGAGAAGTTTCTGTC
GTTAAAAACAGATTTGGAAAGACTGGACTCTAAAGTCTGTTGATTAAAGATGAGCTTTGTCTACT
TCAAAAGTTTGTTTGCTTACCCCTTCAGCCTCCAATTTTTTAAGTGAAAATATAGCTAATAACAT
GTGAAAAGAATAGAAGCTAAGGTTTAGATAAATATTGAGCAGATCTATAGGAAGATTGAACCTGA
ATATTGCCATTATGCTTGACATGGTTTCCAAAAAATGGTACTCCACATATTTCAGTGAGGGTAAG
TATTTTCCTGTTGTCAAGAATAGCATTGTAAAAGCATTTTGTAATAATAAAGAATAGCTTTAATG
ATATGCTTGTAACTAAAATAATTTTGTAATGTATCAAATACATTTAAAACATTAAAATATAATCT
CTATAATAA
Exemplary expanded Human GJB2 Genomic DNA Sequence including certain regulatory
regions
(SEQ ID NO: 6)
GACTGTGAACTTAAGGCACAGCAGAGCTGGGGCTGCTCTTAAGGCCCTGCTGTCTCTCCTCTTAG
TAACAACACCATTTCACATGAAGTGACAGTGGTATCTTTTGTTGCCCTGGAAATGGAATACAACA
ATGGCTTTCCAACTTTTCTGTGGCAGAGACCTACAGACAGAAGTACATTTTACACTGGATCCAGG
ACACACATCAGTCTGAAAACACACACATGAACCAAACGTTTCCTAAAGCATTACTTATCCTTGCT
AATAGCAACACATTCTCATATTCTTTTATACTTCATTTAATTTCATATAAAAAAGAAAAGGAAAG
GAAAGAAATCTATTTCTCAGCCCATTAATAAGGTCAGGAGCAGCAACACCAGACTAGAAGAAAAG
CTTACCTATAGATTTTTCTGCCACCTCTTGAGTGCGTCCAGCTTTCCGACAAGTCTCAGTGCCAT
CTACTGTGCGCTCTGGGTATTGCAATTGCTTTTTTTTTTTTTTTTTTTTTTTTTTTAGAATGAGA
CTAAGTCAGAGAACACAAAGAACTTCTTTCCCCACAGTGGAGATGGCTCTGAAAGCGTTTAAGGA
ATAGCTTAGATGAGTGGCTAACACATTCTCCCGGTTCTGAATTCTAAGACCACAGACTCCATGTC
CAGTCCCCAAAGAGAGGCTTTGCAAGCTACAGAATACCCCTCTGACTGGGACCTCAGGAGCTAAA
CTGACCACGTAATTGGTTCTAGAAAGTGAAACGTTTTAATTTGAAACATCCAAATGAGCATTTTG
TGAAAAGCTACTGCCGTCCATCAAATACAACACAGCCAGGGAGTCATCGCTCTATTGCCCTTGTC
AATCCTACATCTATAGTTTTTTTTGCTACAGCAGTTCATGAGTGTTGACTCTATTCTAACTTGTT
CCAGAAGCCCTTCAAGATGATAGATAGCACAATATTTTTGTAGCCAGAGCTAGAATGTAGAGCTC
TTTTTGGCTTCCTTGTGAATGATCCAGAATTTCCATGTTGGCAAGCCACCATAATTTACAGAATT
TACTTTTTATATTCAATAGAAGTAAAAAAAATTTACCTATTTAAGGAGTTATAGCTCTGGATTCA
TTTCTGACCAAAATGTGCTTTTTGACACAAATACAATTGGAAATGTCTTTGTAATTTATCCACAG
TCTGCCTAGATAATCATAAAAGAACTGCATGGATATATTTGTGAGTAAGAGCACGTGTCCATTCA
GCAAAACCAAGGAGATCAACTAATTCTACCATTGCCTTGAAACGGAGACACATCTAGCAGTTTGA
ATTTCCCCCAAAAGATTGTATGTGTGAAAATAAGAATAGAATGAGGAAAATTTAAAAGCCTATAT
AATAATTTCAGTCACAACTTGGCAATTAGAATTTTATGAGATGTCTTTAATTTGGAAGCAAAGAA
CAATTAAATTATTGAAGGCTGGAATTTTTTTTTAACTCTTTGAATGGAACAACAGATTTTCCCCA
AAAGATTTGACTTTAACAATTTTCAGAAAACATAAGTCAGGGTGTGGTTCAATTACACAGAGAGA
AATTGTAGTGAAATAGTGTTCCCTGTAATAATTACCCACAAAGGAGCACAGTGGAGCCACTCCTG
CATTAAAATTACAGTATCATATGTAAGTTATTATTAATTAACCAGAGATGCCAGGAGCTTGTCAG
TTTCCAACTGCTATTTTGAGGAGAGCTAAAGTTTCTCTTTTTTTGCCAGTTATTATTATTATTAA
TATTTCAACAGCAAGGCAAGAAAAGGGAATGTGGTCCATTAACTAATGGCTCTTGAAAAGACACT
CAATGAATCCAACTTGCCCTAAAACTGCCAAGTGGTAGGACAGTCTCTTCGCGTCTTGCATCATT
TTCTGCCATCACCTACGTGTGATTCGTGAGTCGGAAATTCAACCAAGACATGTTTAATGTATATT
TAGAGCATTCTTCCCGGCGGGAATTCACGGTGCCATTCCATCAGGCAGTTGGCAAGCAGTCACTT
GAAATATTAAGAAATATGATTTGTGTCACACTGATTTATTGCAAAACAGCAACTTCTTTCTTTTT
GGTTCATTTATAAAACAACTGTCAAATTAAAATGCCAAATAGCTTTAAACATTAGCATTTTCACC
TTATAACCTTACAAGTGCATCACTTTAAACATCTGAGTAAAAGTTCAGCTCGATGACAATCACCT
GGGATTTACCTGCATGGTACTAAGCATATATGTAAAAATATTACTGATGGGTATCTCTGGCACTC
TGAAGTGACAAAGTGTAGCCTTCACAGATCTTTGTCAGTTAATCATCAATAGTTACCTGAAAAGT
GCCCACTTGCCATCATTCAAGATCAACCAGGCAGACACCACAGTGAGTTTTCCATCAAAAAACCT
TCTCTATCTGGTCAGTCTCTGCACGTCAATGAGACAAAGGTGTATGCTGCACGCAGCAGTACTAT
CCTAAGCTCCCTGTGTCCTCACCATGGGGCTGGGTGGCTGGGGTGGAGGAACACAGGATTGGGCT
TCAGCTTCTCTAGGGACTGGTACATTAAGAGATGAAGACATAAAAGGTGAGAAAAACATGGTTTA
TTTCCAATGTTTCCATTTCTGTTAAAAGTAATGCTTTCAACAGAAAAAAAATGCAGCAATATAAG
TGTGTAATTTACAAAATAATTTCAGGATTTCTTTAATCATTAATTTGTGGTGTCATCTGTTAACT
GGATTTACGTCTAAGCTCATTTGTAAATAACTTCAAATATCCAAGCCTTCCCTCACCCTTTTCCC
ACCTCACCTCTCCTCCTTCTCCTCCCCTACACTGGAGGACACTATGTACATGCATATAATGTCCT
GCCCTAGAGGAGTCCTGAGCCTACTTGGGAAGAAAACACCAACTCACAGGAAAACAGCAGAAATC
ACACAAAACAGAATAAAAGCAAGCGCTGATCTGTAAGTGAAGACTTAAGTGCTATAGGACTTCCA
GCTACAAATCCTGAAAACACGGAGTGGCTGTGATAATACGACTAGCCAACATCACACAGTAATTT
TGCACATAAGGAGAACTAAATCAAAGAAAACAAGGAAAAGAAAGTTGAGCCTATAATCGTGATAC
AGGCACTAAAATCTCAGGTGACATTTTTCAATGGGGGAAAGTCAGTCAACTTCCGATCTCCAAAC
CATCTTTACTAGCGAGCTTCCCACAATGGTTCTAGAACCTTCCTTCATTCCAACCCAACCAGGAT
TCCAACAGACTCATAAACACCACAGCCTTTGAGAAATTAAAGGGAGAACCCACCAACCGGCGCCC
CACTCCCCACCCCAAGTCACCTCTGGCTCAACCAAGATGCGCTCAGGCCAAGAAAGCTGCCCCAC
CCCACAGGCTTTGCCTGTCATTTTTAACAAGCCGACTCAGCACATCTCTCAGATGGGCCATGCAA
GGCTTTTCGCAGCTCCTGGGGCTTTGCCTCTTCATGAGCAGACACTCCCTCTTAGACTAAGACCT
GGAGCTGGAAAGTAGGTGGTAACCGCGGTACAAAACTCACGCTCGTCCCTGCAGAAACTGCCTAG
GTCGGCCCATGGCCACGGGGCGCCAATTTTTCAAGGAAAAGTCAATGCTAATAATGGTGGCAATC
ACGGGAAATCCATTCTGAGGCCAGATCTGACTTGTCAGGATTAATCATCATTTCCACTTAACTTC
GAACTGACCTGGGTAAAAACGTGAGCGCGAGGGGACCAGGCTGCACCTCTGACCTGGCTCCCCTC
TGCAAAAATCGCGAAGTGGGTGCCCGAGGTGGGGCGGGGGTTGGGGGAGACCTCCCCGGGAGTCC
CCACCCAGCCTGCTCTGCACATCTTAGTCCCTCATCCGCTTGCGCTGTGCAAATCTGTCTTCTGT
CATTTGTATCGCAAGACATCAAAATCCCCAACCAAATGCAAATACTGAGACCTCATAATCTGAGA
CAAAGTTTCACGGTATCCAGAAAGCCCCCAGCAGGTGTGCAGTGCAGAGCCAGCCCCCCAGCGGT
CTTCCGCAGAATCCTATCAGTTTCCCCCTTTCGTGCTGTGTGCATCGAGCAGGAAGGGGCTTGGC
AGGTTTTACCTGCCCTCTTTCCTTTCTGAAAAGTCTGGGCCTCCTCACCCCGAAAGGAGTCACCT
CCTTGCAGTTCCCCAGTTGCGAAAAGAGGAGGAAGTTGGCTGGGCCGGGGGCCGCGGGGGGCACC
CTCCGCAGATGGCGGGACCCCCCTGCCGGCCATGGCAAAAACGAGGCTTGTCTCTCCCACCGCCC
CCAACCTTAGTCCTTGGCACATTGTTGAAAGTAATTGAATAAAATCGGAAATTCGAGAAGGCGTT
CGTTCGGATTGGTGAGATTTTGAGGGGAGAAAGAAGCGGGGACTTCGCCGGCACCAGCGGCGCCC
CCTCCTCGGCCACCGTTAACCCCCATTCCAGAGGGCACTGCCCCGCCACCCAGCCTAGGTCCCCC
TGCGAGAGCCTCGCGGGCCCGCGCAGCCTCCGCGACTCGAACAGATCTTCAGTCCTTGGAGGAAT
GCCTGTTTCTCTAACAATAAAAAATTAAAGAAGCGCTCATAAATGCCAAGTCCTCTCGCACTATG
CGGAGTACAGAGGACAACGACCACAGCCATCCCTGAACCCCGCCCACGGCACAGCGCCGGAGCCG
GGGTCTGGGGCGCCGCTTCCTGGGGGGTCCCGACTCTCAGCCGCCCCCGCTTCACCCGGGCCGCC
AAGGGGCTGGGGGAGGCGGCGCTCGGGGTAACCGGGGGAGACTCAGGGCGCTGGGGGCACTTGGG
GAACTCATGGGGGCTCAAAGGAACTAGGAGATCGGGACCTCGAAGGGGACTTGGGGGGTTCGGGG
CTTTCGGGGGCGGTCGGGGGTTCGCGGACCCGGGAAGCTCTGAGGACCCAGAGGCCGGGCGCGCT
CCGCCCGCGGCGCCGCCCCCTCCGTAACTTTCCCAGTCTCCGAGGGAAGAGGCGGGGTGTGGGGT
GCGGTTAAAAGGCGCCACGGCGGGAGACAGGTGTTGCGGCCCCGCAGCGCCCGCGCGCTCCTCTC
CCCGACTCGGAGCCCCTCGGCGGCGCCCGGCCCAGGACCCGCCTAGGAGCGCAGGAGCCCCAGCG
CAGAGACCCCAACGCCGAGACCCCCGCCCCGGCCCCGCCGCGCTTCCTCCCGACGCAGGTGAGCC
CGCCGGCCCCGGACTGCCCGGCCAGGAACCTGGCGCGGGGAGGGACCGCGAGACCCAGAGCGGTT
GCCCGGCCGCGTGGGTCTCGGGGAACCGGGGGGCTGGACCAACACACGTCCTTGGGCCGGGGGGC
GGGGGCCGCCTTCTGGAGCGGGCGTTTCTGCGGCCGAGCTCCGGAGCTGGAATGGGGCGGCCGGG
GAAGTGGACGCGATGGCACCGCCCGGGGTGCGAGTGGGGCCGGGCGCGCGCGGGAGGGGAAAAAG
GCGCGGGCGAGCCGCCAGCGCGAGGTTTGTGGTGTCGCCGATGTCCCTTCGGGGTACTCTAGCGC
AGCCGCCTGGCTACTTGACCCACTGCCACCAAACGTTTTAAATTCACCGAAAGCTTAGCTTCGAA
GCAAAGCTCCGTTTCGCCGGTGAAGCAGGAAGCCTTCGCTGCAGGAACTGACCTTTACCTCTTGG
AGCGGCTTCTGCAGAAAAATCCCCGGGCAGAGATTTGGGCGGAGTTTGCCTAGAACTAACGCGGA
GCCAGCCGATCCCGGCCTACCCCGGGGCCAAGATTTCAGTGGCTTCCCTTTTTCCTAAACACTTC
ACGAGGGTCTGTTTCCGGGCTGTGCTCCCCGCCTAGAAGGAAAATTTTTAGGACCCTTGTTCGCG
AAGAGGTGGTGTGCGGCTGAGACCCGCGTCCTCAGGACGGTTCCATCAGTGCCTCGATCCTGCCC
CACTGGAGGAGGAAGGCAGCCCGAACAGCGCTCACCTAACTAACAGCTGCTGAGAGCTGGGTTCC
GTGGCCATGCACCTGGGACTGCCTTGAGAAGCGTGGTACGGCCGTGTCCCCATGTGACCTTAGAG
TCCCTTTCGAAACTGCTGTGCACAGTCGGTCACAATTTCAGACACTGGTGAGAAGGGTGGAGGAA
CCCTCTGGGGACAGCCAGGCAAGGTCGACCACCCATCACCTAAGGGTGGAGAAATTTAAGGGGTG
AAGAGTCCCTTTTGCCTTTTCTGGATCCTGGTGATTCACCTAGTGTCTTCCCTAAGGAACTGAAC
CAACTCCTCCGCTGGCCTCTGGCAGCCCTCCAGGCGGTGCAGGATGGCGTGGGCCCGGTAGGAAG
CTGCATGTAACCGCCCAGGGTCGGGAGGCCAGGAGGGCAGCTCCTCCTCTGACTTGAATATTGAA
AACAACTTCGTCCTGCTTCTGAGCCCCTCTTAACCCATGACCCCCTAGCCCATTGGGGAGTAAAT
CTTAATTTACTCCTCTTCCTGAAAAAGGATCTTTAAAACAGGTAGCTTCAACTCAAGCTTTATAA
AATAACAATATAGGGTTTCTCGGAACTGTATTTTTCTCAGCTGATGGTAACTGGACAGGTCTGTA
GAAGGGTGTATGACCTGGGTTTGGCAGGTGGAAGAGGGCAAAGGATAAACCCCTCCTCCTGCAGC
CCCATATTCTTGGCCAGGTGTATTGTTGTAAACCAGGAGAGAGTTTACTTCGGGGAGTATCCTGT
TTTCCACTCAGTGAGGGCCAATGAAGAATGTCTAATTCCATAAGATGCTTTTGTTAAAATCGGAA
TGTTGCTGTCCTCGGTGGTTCTGCTGTTGGGACGGGACTGGCCTGAGCTGTGGGTGCTGTAGCAG
GACAACCAGCTCACCTAAGGGCCTCCCAGTCTGGATTATCAATGGGTCAGTGCTGAACCTGGGCT
AAAATATTGTTTTTTCCAATGATGTTGTCTTTCCCAAGCTCAGTGAAGCTAAATGTTTCACAGGC
CTATGTCAATCTGATGTAACTTTCGTGGCCACCTCTCTCCTGTTAGCCTCTGACCAAGGTGGCAC
TGGATGGTTTCTGCCTGACCTTGGTGCCCCGTGGCAGCGACTGTGGGTCATGAAAGACATTCACT
ACGAGCCTGCTTCTGGAGTCCATCAGAAAACGGGATGCAACTTGCCTAAAATGAGGAGAGGAGGA
TGCTTTTAAGAAAAAGAAGAAGGAGGATTCACTACCAGCTCTGAAGGGTGGAAAAGAGATGATTC
ATCCGGATTGTGGAGAGGGTGGAATCTTGTTTAGGAGAGCGTTGGTTGTGGCAGGCAGGGTGTAA
CTATGAATCAGTGAAGACAATTCACATCCTGGGATGAAAAGAAGGCCATGGGCTCACAGGAGATT
ATCCACTGGCCTCTCCACATCCGCTTGCAGTAAGGAGTGTGGGACTCTCCCAAGCTTCAGCGCTG
AACTGCAATGCAGTGACGTCGCTTAGCTGGGCCAGTAACCGAGGGAGTTGAATTTTCTGTCATTT
TAAAATAATGTGTCTTTTAAGAAACACTTTGAAATTAAAACCACAGCCCACAATTATAATGCACT
GTTGCAGCACTTATCAAAACAGATATGCTAACTGAGCCATCAGTGCCAGCCTGACAGTGAGGCCA
CCAAGCCATCCACAAAGCCTACACGAAAGTCTGTGCTCACAGTGGCTTTTCTCCATGAAGAGGGC
ATTCCTAACCTCTTCCTTTCACGTAGGAGGAAGCAAGGTCCTTTGTAAAATTTTAACTCGGGGTG
CCTCAAATGTAAACTTAACCACTGGTAACAACAGTTTCACTGCTACATGCCACGTCTGTGAAAAT
TCATTCAAGACATTAAGGAAAGTGGCTCAGCAGAGAGACTAGACATCTTATCCTCACGGTTCTCC
TGTACTTGGCCTCTCAGCCTTTGAGCAAGGTTGGCCCAAGCTAGTATCGGCCCCAGTGGTACAGC
CAAAACTTGAGACTGCAAATGGATGCAGCTGTTGAACGCTGAGTAACTTCTGCAGAGTCAGGAAG
ACCCAAGGAAGCTCTGCAGAGGATGCAGGGGTACGGTCAGAACCCCTGAGTGCCTTTCAGCTAAC
GAGGACTTTATGACACTCCCCAGCACAGCAAATTTTTATGATGTGTTTAAAGATTGGGTGAATTA
CTCAGGTGAACAAGCTACTTTTTATCAGAGAACACCTAAAAACACGTTCAAGAGGGTTTGGGAAC
TATACATTTAATCCTATGACAAACTAAGTTGGTTCTGTCTTCACCTGTTTTGGTGAGGTTGTGTA
AGAGTTGGTGTTTGCTCAGGAAGAGATTTAAGCATGCTTGCTTACCCAGACTCAGAGAAGTCTCC
CTGTTCTGTCCTAGCTAGTGATTCCTGTGTTGTGTGCATTCGTCTTTTCCAGAGCAAACCGCCCA
GAGTAGAAGATGGATTGGGGCACGCTGCAGACGATCCTGGGGGGTGTGAACAAACACTCCACCAG
CATTGGAAAGATCTGGCTCACCGTCCTCTTCATTTTTCGCATTATGATCCTCGTTGTGGCTGCAA
AGGAGGTGTGGGGAGATGAGCAGGCCGACTTTGTCTGCAACACCCTGCAGCCAGGCTGCAAGAAC
GTGTGCTACGATCACTACTTCCCCATCTCCCACATCCGGCTATGGGCCCTGCAGCTGATCTTCGT
GTCCACGCCAGCGCTCCTAGTGGCCATGCACGTGGCCTACCGGAGACATGAGAAGAAGAGGAAGT
TCATCAAGGGGGAGATAAAGAGTGAATTTAAGGACATCGAGGAGATCAAAACCCAGAAGGTCCGC
ATCGAAGGCTCCCTGTGGTGGACCTACACAAGCAGCATCTTCTTCCGGGTCATCTTCGAAGCCGC
CTTCATGTACGTCTTCTATGTCATGTACGACGGCTTCTCCATGCAGCGGCTGGTGAAGTGCAACG
CCTGGCCTTGTCCCAACACTGTGGACTGCTTTGTGTCCCGGCCCACGGAGAAGACTGTCTTCACA
GTGTTCATGATTGCAGTGTCTGGAATTTGCATCCTGCTGAATGTCACTGAATTGTGTTATTTGCT
AATTAGATATTGTTCTGGGAAGTCAAAAAAGCCAGTTTAACGCATTGCCCAGTTGTTAGATTAAG
AAATAGACAGCATGAGAGGGATGAGGCAACCCGTGCTCAGCTGTCAAGGCTCAGTCGCTAGCATT
TCCCAACACAAAGATTCTGACCTTAAATGCAACCATTTGAAACCCCTGTAGGCCTCAGGTGAAAC
TCCAGATGCCACAATGGAGCTCTGCTCCCCTAAAGCCTCAAAACAAAGGCCTAATTCTATGCCTG
TCTTAATTTTCTTTCACTTAAGTTAGTTCCACTGAGACCCCAGGCTGTTAGGGGTTATTGGTGTA
AGGTACTTTCATATTTTAAACAGAGGATATCGGCATTTGTTTCTTTCTCTGAGGACAAGAGAAAA
AAGCCAGGTTCCACAGAGGACACAGAGAAGGTTTGGGTGTCCTCCTGGGGTTCTTTTTGCCAACT
TTCCCCACGTTAAAGGTGAACATTGGTTCTTTCATTTGCTTTGGAAGTTTTAATCTCTAACAGTG
GACAAAGTTACCAGTGCCTTAAACTCTGTTACACTTTTTGGAAGTGAAAACTTTGTAGTATGATA
GGTTATTTTGATGTAAAGATGTTCTGGATACCATTATATGTTCCCCCTGTTTCAGAGGCTCAGAT
TGTAATATGTAAATGGTATGTCATTCGCTACTATGATTTAATTTGAAATATGGTCTTTTGGTTAT
GAATACTTTGCAGCACAGCTGAGAGGCTGTCTGTTGTATTCATTGTGGTCATAGCACCTAACAAC
ATTGTAGCCTCAATCGAGTGAGACAGACTAGAAGTTCCTAGTGATGGCTTATGATAGCAAATGGC
CTCATGTCAAATATTTAGATGTAATTTTGTGTAAGAAATACAGACTGGATGTACCACCAACTACT
ACCTGTAATGACAGGCCTGTCCAACACATCTCCCTTTTCCATGACTGTGGTAGCCAGCATCGGAA
AGAACGCTGATTTAAAGAGGTCGCTTGGGAATTTTATTGACACAGTACCATTTAATGGGGAGGAC
AAAATGGGGCAGGGGAGGGAGAAGTTTCTGTCGTTAAAAACAGATTTGGAAAGACTGGACTCTAA
AGTCTGTTGATTAAAGATGAGCTTTGTCTACTTCAAAAGTTTGTTTGCTTACCCCTTCAGCCTCC
AATTTTTTAAGTGAAAATATAGCTAATAACATGTGAAAAGAATAGAAGCTAAGGTTTAGATAAAT
ATTGAGCAGATCTATAGGAAGATTGAACCTGAATATTGCCATTATGCTTGACATGGTTTCCAAAA
AATGGTACTCCACATATTTCAGTGAGGGTAAGTATTTTCCTGTTGTCAAGAATAGCATTGTAAAA
GCATTTTGTAATAATAAAGAATAGCTTTAATGATATGCTTGTAACTAAAATAATTTTGTAATGTA
TCAAATACATTTAAAACATTAAAATATAATCTCTATAATAATTTAAAATCTAATATGGTTTTAAT
AGAACAGCAAATTTTAATTTCATCTATCACTTTTTATATAAATACATTAATGTTTTATATTTCAT
AACACCAATGGGTAAGTTGCCAGAGTGTCTGACCCCATTCTGCCCCAGTTACAGAAAAGCTTCTG
TCACCAGAAAGTTTGGTGGGGAAGGAAGGGAGGAAGATGATTTCTACCTAACCCCGTGCCCACCT
CTACCAGGTTTTTGAGGCATATCAGTCTATGGACAATGTGGTGTTTGGTCTGGAAACGTACCTTG
GTGAATGCTGAGTTGGCTGGACATGACCCGTTTAGCTCCTGGATGAATCCCAGAAGTGGACCTTC
AAAATGTTACTCATAGCATGACCTTGGCTCACTGCAACCTCTGCCTCCCAGGCTCAAGCGATCCT
CCCACCTCAGCGTCCCAAGTAGCTGGGACCACTGGAGTGTGCCACCACACTCCACTAATTTTTTC
ATTTTTTGTAGAAACGAGGTCCCACTATATTGCCCAGTCTGGTCTCGAACTCCTGGGCTGAAGGG
ATCCCCCTGCCTCAGTCTCCTAAAGTGCAAGGATTACAGGCATGGGCCACCGCACCTGGCCTGAA
ACTGCTTTTTATTCCTCAGTGCCCACTTCCATGGGAAATAAGCCTGCCAGGTCAGCCTGTCCCCA
TGGGAGTGACTGCCTGCTACCCCCACAGGCTTGCCCGGCCCTCGTGAGCCTCTCCCAGAGACACC
ACCAACAGTTCTGTTCTTTCATGGTACAAGATTTCCATCCAAGGATTTCAAAGCATTTCACACAT
CAATAATTAGAAGTATTTTCATAGAGGACCATACACTTTTAAAATGGATTTCAAAGAACAAAAAC
CAGTCAACTATCACCCAGGTAATAGAAAATGGGAAATGGTTTCTACCTGACTTCCAAAATGCTCT
GCACATAGACTGTGAAAATAGGATTTTTTAAGCTGGGTGCAGAGGCTTATACCTATAATCCCAAC
ACTTTGGGAGGCTGAGACGAGAGGATCACTTGAGCCCAGGAGTTCAAAACCAGCCTGGGCAATAT
AGGGAGACATTGTTTCTATAAAAAATAAAAATGTTAGCCAGGCAGGCGTGGTAACATGTGCCTGT
AGTCTCAGCTACTCAGGAGGCTGAGGTGGGAAGATTGCTTGAACCTGGGAGGTCCATGCTGCAGT
GAGCTGAGATTGTGCCACTGCACTCCAGCCTAGGCGACAGCAAGATCCTGTCCCAAACAACAACA
ACATCAAAAAACACAGAACTTTTAAAATAAGTACATTCACTTCTACAAGCTATGTAGATTATTAC
TCTCAAGCTATTAAAAGACCAAGCCAAAATAATTATGGGCTACTCTCGACCACTTGTAGGAATGG
ATAGAGAGGTCTGGTCACATGCCTGGAAATTAGAGCTTGAGCTCTGAAAATGATAATCCTGACTA
TATCTCAAAGCATCAGTCTGCACTTTGTATGGAGCAAGAAAAAGCCTTGTGGAAGCGGCCTCCCA
CCCAGCCGAGCCCTCGGCGTGGACAAGCTCTGCTTTTTATGAGCAGTGGGTGCAGCCTCGCTGCT
CCCTCCTCCTGTCAAAAGACAGTCACAGCTGGGGTGAGCAGATCGGGCCCACTTGGGAGGCCCCA
AGGAATATGCTGCAGGGGTCGGGCCTGAGCCACCCCCACGGGTTGGTCTTTGACAACTAGAGAGC
AGCTGAGAGGTGGGTAAAAGCTCACTCACTTACCCTGACCTCAGTGTCCTCATCTTAAAATGGGT
TTCCTGAATCTTTCCCCGGCTTAGTGGCAATGAAATAAGATAATTTATGTAAACGTTCTCCACAT
AGTAAAGCACTAAGTAACATATGACTGTCATCTGTTTTCCACTAGACAGATCCCAACCTGGAAGA
GTGACAGATGGTATTTCAGATACAAGTGACTCAAGCAAAGCTTGATAAACTGGGGGCTGGAAAAA
AATGCACATTTACACAAAGCCTGGAGTAACTGC

TABLE 1
Nucleotides Corresponding to Introns and Exons in SEQ ID NO: 5

| Element | Nucleotides | Element | Nucleotides |
|---|---|---|---|
| Isoform 1 Exon 1 | 1-156 | Isoform 1 Intron 1 | 157-3335 |
| Isoform 1 Exon 2 | 3336-5469 | | |
| Isoform X1 Exon 1 | 795-978 | Isoform X1 Intron 1 | 979-3335 |
| Isoform X1 Exon 2 | 3336-5469 | | |

TABLE 2
Nucleotides Corresponding to Introns and Exons in SEQ ID NO: 6

| Element | Nucleotides | Element | Nucleotides |
|---|---|---|---|
| Isoform 1 Exon 1 | 5,038-5,193 | Isoform 1 Intron 1 | 5,194-8,372 |
| Isoform 1 Exon 2 | 8,373-10,506 | | |
| Isoform X1 Exon 1 | 5,832-6,015 | Isoform X1 Intron 1 | 6,016-8,372 |
| Isoform X1 Exon 2 | 8,373-10,506 | | |

The present disclosure recognizes that certain changes to a polynucleotide sequence will not impact its expression or a protein encoded by said polynucleotide. In some embodiments, a polynucleotide comprises a GJB2 gene having one or more silent mutations. In some embodiments, the disclosure provides a polynucleotide that comprises a GJB2 gene having one or more silent mutations, e.g., a GJB2 gene having a sequence different from SEQ ID NO: 1, 2, 3, 4, 5 or 6 but encoding the same amino acid sequence as a functional GJB2 gene. In some embodiments, the disclosure provides a polynucleotide that comprises a GJB2 gene having a sequence different from SEQ ID NO: 1, 2, 3, 4, 5 or 6 that encodes an amino acid sequence including one or more mutations (e.g., a different amino acid sequence when compared to that produced from a functional GJB2 gene), where the one or more mutations are conservative amino acid substitutions. In some embodiments, the disclosure provides a polynucleotide that comprises a GJB2 gene having a sequence different from SEQ ID NO: 1, 2, 3, 4, 5 or 6 that encodes an amino acid sequence including one or more mutations (e.g., a different amino acid sequence when compared to that produced from a functional GJB2 gene), where the one or more mutations are not within a characteristic portion of a GJB2 gene or an encoded connexin 26 protein. In some embodiments, a polynucleotide in accordance with the present disclosure comprises a GJB2 gene that is at least 85%, at least 90%, at least 95%, at least 98%, or at least 99% identical to a sequence of SEQ ID NO: 1, 2, 3, 4, 5 or 6 In some embodiments, a polynucleotide in accordance with the present disclosure comprises a GJB2 gene that is identical to the sequence of SEQ ID NO: 1, 2, 3, 4, 5 or 6. As can be appreciated in the art, SEQ ID NO: 1, 2, 3, 4, 5 or 6 can be optimized (e.g., codon optimized) to achieve increased or optimal expression in an animal, e.g., a mammal, e.g., a human.

### Polypeptides Encoded by GJB2 Gene

Among other things, the present disclosure provides polypeptides encoded by a GJB2 gene or characteristic portion thereof. In some embodiments, a GJB2 gene is a mammalian GJB2 gene. In some embodiments, a GJB2 gene is a murine GJB2 gene. In some embodiments, a GJB2 gene is a primate GJB2 gene. In some embodiments, a GJB2 gene is a human GJB2 gene.

In some embodiments, a polypeptide comprises a connexin 26 protein or characteristic portion thereof. In some embodiments, a connexin 26 protein or characteristic portion thereof is mammalian connexin 26 protein or characteristic portion thereof, e.g., primate connexin 26 protein or characteristic portion thereof. In some embodiments, a connexin 26 protein or characteristic portion thereof is a human connexin 26 protein or characteristic portion thereof.

In some embodiments, a polypeptide provided herein comprises post-translational modifications. In some embodiments, a connexin 26 protein or characteristic portion thereof provided herein comprises post-translational modifications. In some embodiments, post-translational modifications can comprise but is not limited to glycosylation (e.g., N-linked glycosylation, O-linked glycosylation), phosphorylation, acetylation, amidation, hydroxylation, methylation, ubiquitylation, sulfation, and/or a combination thereof. An exemplary human connexin 26 protein sequence is or includes the sequence of SEQ ID NO: 7.

Exemplary Human Connexin 26 Protein Sequence
(SEQ ID NO: 7)
MDWGTLQTILGGVNKHSTSIGKIWLTVLFIFRIMILVVAAKEVWGDEQAD
FVCNTLQPGCKNVCYDHYFPISHIRLWALQLIFVSTPALLVAMHVAYRRH
EKKRKFIKGEIKSEFKDIEEIKTQKVRIEGSLWWTYTSSIFFRVIFEAAF
MYVFYVMYDGFSMQRLVKCNAWPCPNTVDCFVSRPTEKTVFTVFMIAVSG
ICILLNVTELCYLLIRYCSGKSKKPV

The present disclosure recognizes that certain mutations in an amino acid sequence of a polypeptide described herein (e.g., including connexin 26 or a characteristic portion thereof) will not impact the expression, folding, or activity of the polypeptide. In some embodiments, a polypeptide (e.g., including connexin 26 or a characteristic portion thereof) includes one or more mutations, where the one or more mutations are conservative amino acid substitutions. In some embodiments, a polypeptide in accordance with the present disclosure comprises a connexin 26 or a characteristic portion thereof that is at least 85%, at least 90%, at least 95%, at least 98%, or at least 99% identical to a sequence of SEQ ID NO: 7. In some embodiments, a polypeptide in accordance with the present disclosure comprises a connexin 26 or a characteristic portion thereof that is identical to the sequence of SEQ ID NO: 7. In some embodiments, a polypeptide in accordance with the present disclosure comprises a connexin 26 or a characteristic portion thereof that is at least 85%, at least 90%, at least 95%, at least 98%, or at least 99% identical to a sequence of SEQ ID NO: 7. In some embodiments, a polypeptide in accordance with the present disclosure comprises a connexin 26 protein or a characteristic portion thereof that is identical to the sequence of SEQ ID NO: 7.

### Constructs

Among other things, the present disclosure provides that some polynucleotides as described herein are polynucleotide constructs. Polynucleotide constructs according to the present disclosure include all those known in the art, including cosmids, plasmids (e.g., naked or contained in liposomes) and viral constructs (e.g., lentiviral, retroviral, adenoviral, and adeno-associated viral constructs) that incorporate a polynucleotide comprising a GJB2 gene or characteristic portion thereof. Those of skill in the art will be capable of selecting suitable constructs, as well as cells, for making any of the polynucleotides described herein. In some embodiments, a construct is a plasmid (i.e., a circular DNA molecule that can autonomously replicate inside a cell). In some embodiments, a construct can be a cosmid (e.g., pWE or sCos series).

In some embodiments, a construct is a viral construct. In some embodiments, a viral construct is a lentivirus, retrovirus, adenovirus, or adeno-associated virus construct. In some embodiments, a construct is an adeno-associated virus (AAV) construct (see, e.g., Asokan et al., Mol. Ther. 20: 699-7080, 2012, which is incorporated in its entirety herein by reference). In some embodiments, a viral construct is an adenovirus construct. In some embodiments, a viral construct may also be based on or derived from an alphavirus. Alphaviruses include Sindbis (and VEEV) virus, Aura virus, Babanki virus, Barmah Forest virus, Bebaru virus, Cabassou virus, Chikungunya virus, Eastern equine encephalitis virus, Everglades virus, Fort Morgan virus, Getah virus, Highlands J virus, Kyzylagach virus, Mayaro virus, Me Tri virus, Middelburg virus, Mosso das Pedras virus, Mucambo virus, Ndumu virus, O'nyong-nyong virus, Pixuna virus, Rio Negro virus, Ross River virus, Salmon pancreas disease virus, Semliki Forest virus, Southern elephant seal virus, Tonate virus, Trocara virus, Una virus, Venezuelan equine encephalitis virus, Western equine encephalitis virus, and Whataroa virus. Generally, the genome of such viruses encode nonstructural (e.g., replicon) and structural proteins (e.g., capsid and envelope) that can be translated in the cytoplasm of the host cell. Ross River virus, Sindbis virus, Semliki Forest virus (SFV), and Venezuelan equine encephalitis virus (VEEV) have all been used to develop viral constructs for coding sequence delivery. Pseudotyped viruses may be formed by combining alphaviral envelope glycoproteins and retroviral capsids. Examples of alphaviral constructs can be found in U.S. Publication Nos. 20150050243, 20090305344, and 20060177819; constructs and methods of their making are incorporated herein by reference to each of the publications in its entirety.

Constructs provided herein can be of different sizes. In some embodiments, a construct is a plasmid and can include a total length of up to about 1 kb, up to about 2 kb, up to about 3 kb, up to about 4 kb, up to about 5 kb, up to about 6 kb, up to about 7 kb, up to about 8 kb, up to about 9 kb, up to about 10 kb, up to about 11 kb, up to about 12 kb, up to about 13 kb, up to about 14 kb, or up to about 15 kb. In some embodiments, a construct is a plasmid and can have a total length in a range of about 1 kb to about 2 kb, about 1 kb to about 3 kb, about 1 kb to about 4 kb, about 1 kb to about 5 kb, about 1 kb to about 6 kb, about 1 kb to about 7 kb, about 1 kb to about 8 kb, about 1 kb to about 9 kb, about 1 kb to about 10 kb, about 1 kb to about 11 kb, about 1 kb to about 12 kb, about 1 kb to about 13 kb, about 1 kb to about 14 kb, or about 1 kb to about 15 kb.

In some embodiments, a construct is a viral construct and can have a total number of nucleotides of up to 10 kb. In some embodiments, a viral construct can have a total number of nucleotides in the range of about 1 kb to about 2 kb, 1 kb to about 3 kb, about 1 kb to about 4 kb, about 1 kb to about 5 kb, about 1 kb to about 6 kb, about 1 kb to about 7 kb, about 1 kb to about 8 kb, about 1 kb to about 9 kb, about 1 kb to about 10 kb, about 2 kb to about 3 kb, about 2 kb to about 4 kb, about 2 kb to about 5 kb, about 2 kb to about 6 kb, about 2 kb to about 7 kb, about 2 kb to about 8 kb, about 2 kb to about 9 kb, about 2 kb to about 10 kb, about 3 kb to about 4 kb, about 3 kb to about 5 kb, about 3 kb to about 6 kb, about 3 kb to about 7 kb, about 3 kb to about 8 kb, about 3 kb to about 9 kb, about 3 kb to about 10 kb, about 4 kb to about 5 kb, about 4 kb to about 6 kb, about 4 kb to about 7 kb, about 4 kb to about 8 kb, about 4 kb to about 9 kb, about 4 kb to about 10 kb, about 5 kb to about 6 kb, about 5 kb to about 7 kb, about 5 kb to about 8 kb, about 5 kb to about 9 kb, about 5 kb to about 10 kb, about 6 kb to about 7 kb, about 6 kb to about 8 kb, about 6 kb to about 9 kb, about 6 kb to about 10 kb, about 7 kb to about 8 kb, about 7 kb to about 9 kb, about 7 kb to about 10 kb, about 8 kb to about 9 kb, about 8 kb to about 10 kb, or about 9 kb to about 10 kb.

In some embodiments, a construct is a lentivirus construct and can have a total number of nucleotides of up to 8 kb. In some examples, a lentivirus construct can have a total number of nucleotides of about 1 kb to about 2 kb, about 1 kb to about 3 kb, about 1 kb to about 4 kb, about 1 kb to about 5 kb, about 1 kb to about 6 kb, about 1 kb to about 7 kb, about 1 kb to about 8 kb, about 2 kb to about 3 kb, about 2 kb to about 4 kb, about 2 kb to about 5 kb, about 2 kb to about 6 kb, about 2 kb to about 7 kb, about 2 kb to about 8 kb, about 3 kb to about 4 kb, about 3 kb to about 5 kb, about 3 kb to about 6 kb, about 3 kb to about 7 kb, about 3 kb to about 8 kb, about 4 kb to about 5 kb, about 4 kb to about 6 kb, about 4 kb to about 7 kb, about 4 kb to about 8 kb, about 5 kb to about 6 kb, about 5 kb to about 7 kb, about 5 kb to about 8 kb, about 6 kb to about 8 kb, about 6 kb to about 7 kb, or about 7 kb to about 8 kb

In some embodiments, a construct is an adenovirus construct and can have a total number of nucleotides of up to 8 kb. In some embodiments, an adenovirus construct can have a total number of nucleotides in the range of about 1 kb to about 2 kb, about 1 kb to about 3 kb, about 1 kb to about 4 kb, about 1 kb to about 5 kb, about 1 kb to about 6 kb, about 1 kb to about 7 kb, about 1 kb to about 8 kb, about 2 kb to about 3 kb, about 2 kb to about 4 kb, about 2 kb to about 5 kb, about 2 kb to about 6 kb, about 2 kb to about 7 kb, about 2 kb to about 8 kb, about 3 kb to about 4 kb, about 3 kb to about 5 kb, about 3 kb to about 6 kb, about 3 kb to about 7 kb, about 3 kb to about 8 kb, about 4 kb to about 5 kb, about 4 kb to about 6 kb, about 4 kb to about 7 kb, about 4 kb to about 8 kb, about 5 kb to about 6 kb, about 5 kb to about 7 kb, about 5 kb to about 8 kb, about 6 kb to about 7 kb, about 6 kb to about 8 kb, or about 7 kb to about 8 kb.

Any of the constructs described herein can further include a control sequence, e.g., a control sequence selected from the group of a transcription initiation sequence, a transcription termination sequence, a promoter sequence, an enhancer sequence, an RNA splicing sequence, a polyadenylation (poly(A)) sequence, a Kozak consensus sequence, and/or additional untranslated regions which may house pre- or post-transcriptional regulatory and/or control elements. In some embodiments, a promoter can be a native promoter, a constitutive promoter, an inducible promoter, and/or a tissue-specific promoter. Non-limiting examples of control sequences are described herein.

### AAV Particles

Among other things, the present disclosure provides AAV particles that comprise a construct encoding a GJB2 gene or characteristic portion thereof described herein, and a capsid described herein. In some embodiments, AAV particles can be described as having a serotype, which is a description of the construct strain and the capsid strain. For example, in some embodiments an AAV particle may be described as AAV2, wherein the particle has an AAV2 capsid and a construct that comprises characteristic AAV2 Inverted Terminal Repeats (ITRs). In some embodiments, an AAV particle may be described as a pseudotype, wherein the capsid and construct are derived from different AAV strains, for example, AAV2/9 would refer to an AAV particle that comprises a construct utilizing the AAV2 ITRs and an AAV9 capsid. In some aspects, an AAV capsid is an Anc80 capsid (e.g., an Anc80L65 capsid).

### AAV Construct

The present disclosure provides polynucleotide constructs that comprise a GJB2 gene or characteristic portion thereof. In some embodiments described herein, a polynucleotide comprising a GJB2 gene or characteristic portion thereof can be included in an AAV particle.

In some embodiments, a polynucleotide construct comprises one or more components derived from or modified from naturally occurring AAV genomic construct. In some embodiments, a sequence derived from an AAV construct is an AAV1 construct, an AAV2 construct, an AAV3 construct, an AAV4 construct, an AAV5 construct, an AAV6 construct, an AAV7 construct, an AAV8 construct, an AAV9 construct, an AAV2.7m8 construct, an AAV8BP2 construct, an AAV293 construct, or AAV Anc80 construct. Additional exemplary AAV constructs that can be used herein are known in the art. See, e.g., Kanaan et al., Mol. Ther. Nucleic Acids 8:184-197, 2017; Li et al., Mol. Ther. 16(7): 1252-1260, 2008; Adachi et al., Nat. Commun. 5: 3075, 2014; Isgrig et al., Nat. Commun. 10(1): 427, 2019; and Gao et al., J. Virol. 78(12): 6381-6388, 2004; each of which is incorporated in its entirety herein by reference.

In some embodiments, provided constructs comprise coding sequence, e.g., a GJB2 gene or a characteristic portion thereof, one or more regulatory and/or control sequences, and optionally 5′ and 3′ AAV derived inverted terminal repeats (ITRs). In some embodiments wherein a 5′ and 3′ AAV derived ITR is utilized, the polynucleotide construct may be referred to as a recombinant AAV (rAAV) construct. In some embodiments, provided rAAV constructs are packaged into an AAV capsid to form an AAV particle. In some aspects, an AAV capsid is an Anc80 capsid (e.g., an Anc80L65 capsid).

In some embodiments, AAV derived sequences (which are comprised in a polynucleotide construct) typically include the cis-acting 5′ and 3′ ITR sequences (see, e.g., B. J. Carter, in “Handbook of Parvoviruses,” ed., P. Tijsser, CRC Press, pp. 155 168, 1990, which is incorporated herein by reference in its entirety). Typical AAV2-derived ITR sequences are about 145 nucleotides in length. In some embodiments, at least 80% of a typical ITR sequence (e.g., at least 85%, at least 90%, or at least 95%) is incorporated into a construct provided herein. The ability to modify these ITR sequences is within the skill of the art. (See, e.g., texts such as Sambrook et al., “Molecular Cloning. A Laboratory Manual”, 2d ed., Cold Spring Harbor Laboratory, New York, 1989; and K. Fisher et al., J Virol. 70:520 532, 1996, each of which is incorporated in its entirety by reference). In some embodiments, any of the coding sequences and/or constructs described herein are flanked by 5′ and 3′ AAV ITR sequences. The AAV ITR sequences may be obtained from any known AAV, including presently identified AAV types.

In some embodiments, polynucleotide constructs described in accordance with this disclosure and in a pattern known to the art (see, e.g., Asokan et al., Mol. Ther. 20: 699-7080, 2012, which is incorporated herein by reference in its entirety) are typically comprised of, a coding sequence or a portion thereof, at least one and/or control sequence, and optionally 5′ and 3′ AAV inverted terminal repeats (ITRs). In some embodiments, provided constructs can be packaged into a capsid to create an AAV particle. An AAV particle may be delivered to a selected target cell. In some embodiments, provided constructs comprise an additional optional coding sequence that is a nucleic acid sequence (e.g., inhibitory nucleic acid sequence), heterologous to the construct sequences, which encodes a polypeptide, protein, functional RNA molecule (e.g., miRNA, miRNA inhibitor) or other gene product, of interest. In some embodiments, a nucleic acid coding sequence is operatively linked to and/or control components in a manner that permits coding sequence transcription, translation, and/or expression in a cell of a target tissue.

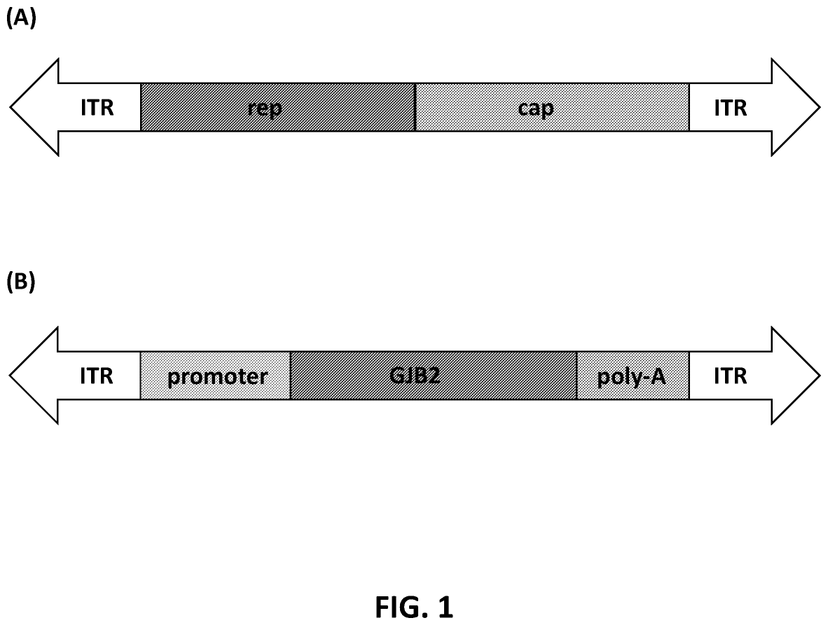

As shown in FIG. 1, panel (A), an unmodified AAV endogenous genome includes two open reading frames, “cap” and “rep,” which are flanked by ITRs. As shown in FIG. 1, panel (B), exemplary rAAV constructs similarly include ITRs flanking a coding region, e.g., a coding sequence (e.g., a GJB2 gene). In some embodiments, an rAAV construct also comprises conventional control elements that are operably linked to the coding sequence in a manner that permits its transcription, translation and/or expression in a cell transfected with the plasmid construct or infected with the virus produced by the disclosure. In some embodiments, an rAAV construct optionally comprises a promoter (shown in FIG. 1, panel (B)), an enhancer, an untranslated region (e.g., a 5′ UTR, 3′ UTR), a Kozak sequence, an internal ribosomal entry site (IRES), splicing sites (e.g., an acceptor site, a donor site), a polyadenylation site (shown in FIG. 1, panel (B)), or any combination thereof.

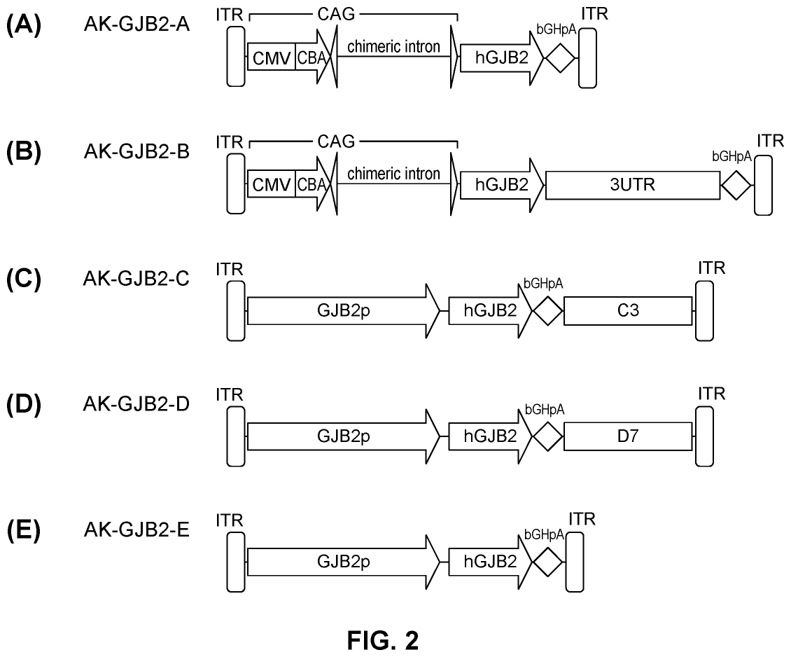

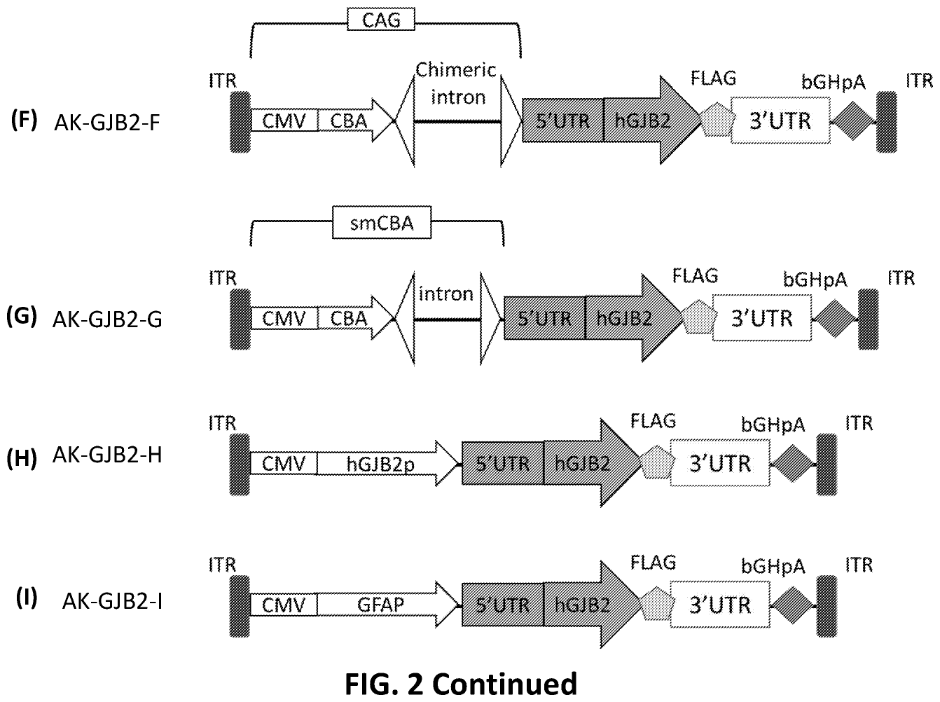

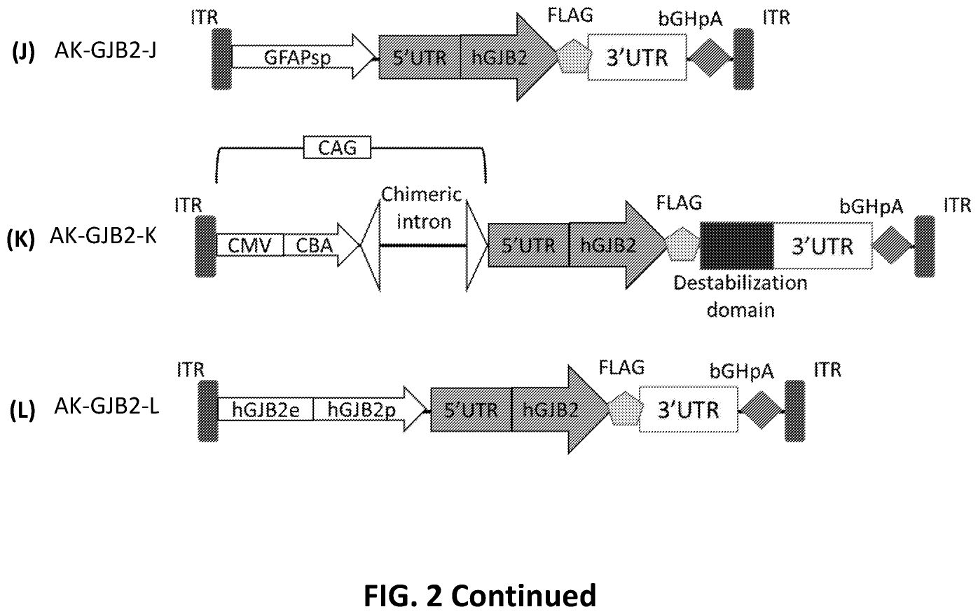

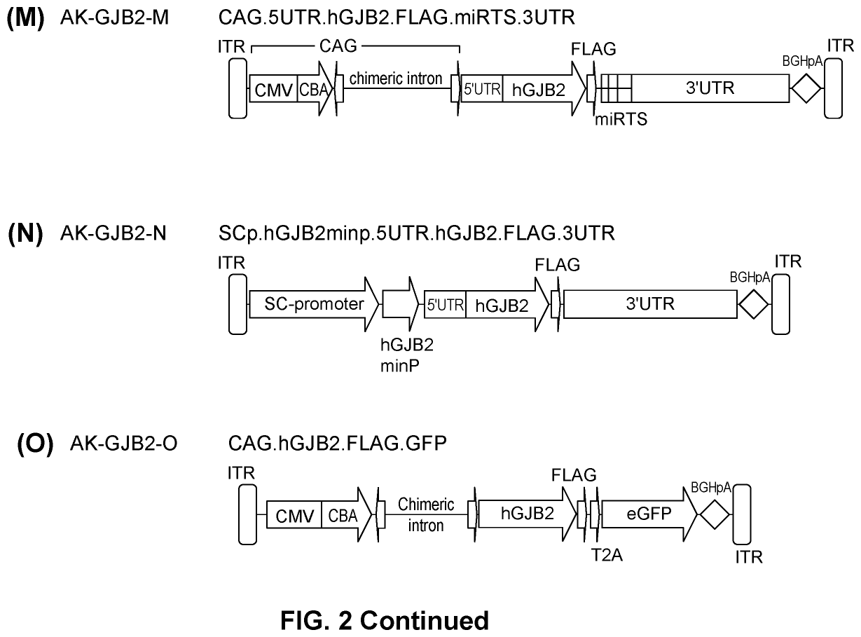

In some aspects, an rAAV construct comprises a 5′ ITR, a promoter, a hGJB2 gene, a polyA, and a 3′ ITR (shown in FIGS. 2A and 2E). In some aspects, an rAAV construct comprises a 5′ ITR, a promoter, a hGJB2 gene, a 3′ UTR, a polyA, and a 3′ ITR (shown in FIG. 2B). In some aspects, an rAAV construct comprises a 5′ ITR, a promoter, a hGJB2 gene, a C3 domain, a polyA, and a 3′ ITR (shown in FIG. 2C). In some aspects, an rAAV construct comprises a 5′ ITR, a promoter, a hGJB2 gene, a D7 domain, a polyA, and a 3′ ITR (shown in FIG. 2D). In some aspects, an rAAV construct comprises a 5′ ITR, a promoter, a 5′ UTR, a hGJB2 gene, an optional FLAG tag, a 3′UTR, a polyA, and a 3′ ITR (shown in FIGS. 2F-2J, 2L, and 2N). In some aspects, an rAAV construct comprises a 5′ ITR, a promoter, a 5′ UTR, a hGJB2 gene, an optional FLAG tag, a 3′ UTR, a microRNA regulatory target site, a polyA, and a 3′ ITR (shown in FIG. 2M). Such additional elements are described further herein.

In some embodiments, a construct is a rAAV construct. In some embodiments, an rAAV construct can include at least 500 bp, at least 1 kb, at least 1.5 kb, at least 2 kb, at least 2.5 kb, at least 3 kb, at least 3.5 kb, at least 4 kb, or at least 4.5 kb. In some embodiments, an AAV construct can include at most 7.5 kb, at most 7 kb, at most 6.5 kb, at most 6 kb, at most 5.5 kb, at most 5 kb, at most 4.5 kb, at most 4 kb, at most 3.5 kb, at most 3 kb, or at most 2.5 kb. In some embodiments, an AAV construct can include about 1 kb to about 2 kb, about 1 kb to about 3 kb, about 1 kb to about 4 kb, about 1 kb to about 5 kb, about 2 kb to about 3 kb, about 2 kb to about 4 kb, about 2 kb to about 5 kb, about 3 kb to about 4 kb, about 3 kb to about 5 kb, or about 4 kb to about 5 kb.

Any of the constructs described herein can further include regulatory and/or control sequences, e.g., a control sequence selected from the group of a transcription initiation sequence, a transcription termination sequence, a promoter sequence, an enhancer sequence, an RNA splicing sequence, a polyadenylation (poly(A)) sequence, a Kozak consensus sequence, and/or any combination thereof. In some embodiments, a promoter can be a native promoter, a constitutive promoter, an inducible promoter, and/or a tissue-specific promoter. Non-limiting examples of control sequences are described herein.

### Exemplary Construct Components

Inverted Terminal Repeat Sequences (ITRs)

AAV derived sequences of a construct typically comprises the cis-acting 5′ and 3′ ITRs (See, e.g., B. J. Carter, in “Handbook of Parvoviruses”, ed., P. Tijsser, CRC Press, pp. 155 168 (1990), which is incorporated in its entirety herein by reference). Generally, ITRs are able to form a hairpin. The ability to form a hairpin can contribute to an ITRs ability to self-prime, allowing primase-independent synthesis of a second DNA strand. ITRs also play a role in integration of AAV construct (e.g., a coding sequence, e.g., a GJB2 gene) into a genome of a subject's cell. ITRs can also aid in efficient encapsidation of an AAV construct in an AAV particle.

An rAAV particle (e.g., an AAV2/Anc80 particle) of the present disclosure can comprise a rAAV construct comprising a coding sequence (e.g., GJB2 gene) and associated elements flanked by a 5′ and a 3′ AAV ITR sequences. In some embodiments, an ITR is or comprises about 145 nucleic acids. In some aspects, an ITR is or comprises about 119 nucleic acids. In some aspects, an ITR is or comprises about 130 nucleic acids. In some embodiments, all or substantially all of a sequence encoding an ITR is used. An AAV ITR sequence may be obtained from any known AAV, including presently identified mammalian AAV types. In some embodiments an ITR is an AAV2 ITR.

An example of a construct molecule employed in the present disclosure is a “cis-acting” construct containing a transgene, in which the selected transgene sequence and associated regulatory elements are flanked by 5′ or “left” and 3′ or “right” AAV ITR sequences. 5′ and left designations refer to a position of an ITR sequence relative to an entire construct, read left to right, in a sense direction. For example, in some embodiments, a 5′ or left ITR is an ITR that is closest to a promoter (as opposed to a polyadenylation sequence) for a given construct, when a construct is depicted in a sense orientation, linearly. Concurrently, 3′ and right designations refer to a position of an ITR sequence relative to an entire construct, read left to right, in a sense direction. For example, in some embodiments, a 3′ or right ITR is an ITR that is closest to a polyadenylation sequence (as opposed to a promoter sequence) for a given construct, when a construct is depicted in a sense orientation, linearly. ITRs as provided herein are depicted in 5′ to 3′ order in accordance with a sense strand. Accordingly, one of skill in the art will appreciate that a 5′ or “left” orientation ITR can also be depicted as a 3′ or “right” ITR when converting from sense to antisense direction. Further, it is well within the ability of one of skill in the art to transform a given sense ITR sequence (e.g., a 5′/left AAV ITR) into an antisense sequence (e.g., 3′/right ITR sequence). One of ordinary skill in the art would understand how to modify a given ITR sequence for use as either a 5′/left or 3′/right ITR, or an antisense version thereof.

For example, in some embodiments an ITR (e.g., a 5′ ITR) can have a sequence according to SEQ ID NO: 8. In some embodiments, an ITR (e.g., a 3′ ITR) can have a sequence according to SEQ ID NO: 9. In some embodiments, an ITR includes one or more modifications, e.g., truncations, deletions, substitutions or insertions, as is known in the art. In some embodiments, an ITR comprises fewer than 145 nucleotides, e.g., 127, 130, 134 or 141 nucleotides. For example, in some embodiments, an ITR comprises 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143 144, or 145 nucleotides. In some aspects, the ITR comprises about 119 nucleotides. In some aspects, the ITR comprises about 130 nucleotides. In some embodiments an ITR (e.g., a 5′ ITR) can have a sequence according to SEQ ID NO: 52. In some embodiments, an ITR (e.g., a 3′ ITR) can have a sequence according to SEQ ID NO: 53.

A non-limiting example of 5′ AAV ITR sequences includes SEQ ID NO: 8 or 52. A non-limiting example of 3′ AAV ITR sequences includes SEQ ID NO: 9 or 53. In some embodiments, the 5′ and a 3′ AAV ITRs (e.g., SEQ ID NOs: 8 and 9, or SEQ ID NOs: 52 and 53) flank a portion of a coding sequence, e.g., all or a portion of a GJB2 gene (e.g., SEQ ID NO: 1, 2, 3, 4, 5, or 6). The ability to modify these ITR sequences is within the skill of the art. (See, e.g., texts such as Sambrook et al. “Molecular Cloning. A Laboratory Manual”, 2d ed., Cold Spring Harbor Laboratory, New York (1989); and K. Fisher et al., J Virol., 70:520 532 (1996), each of which is incorporated in its entirety herein by reference). In some embodiments, a 5′ ITR sequence is at least 85%, 90%, 95%, 98% or 99% identical to a 5′ ITR sequence represented by SEQ ID NO: 8. In some embodiments, a 3′ ITR sequence is at least 85%, 90%, 95%, 98% or 99% identical to a 3′ ITR sequence represented by SEQ ID NO: 9. In some embodiments, a 5′ ITR sequence is at least 85%, 90%, 95%, 98% or 99% identical to a 5′ ITR sequence represented by SEQ ID NO: 52. In some embodiments, a 3′ ITR sequence is at least 85%, 90%, 95%, 98% or 99% identical to a 3′ ITR sequence represented by SEQ ID NO: 53.

Exemplary 5′ AAV ITR
(SEQ ID NO: 8)
TTGGCCACTCCCTCTCTGCGCGCTCGCTCGCTCACTGAGGCCGCCCGGGC
AAAGCCCGGGCGTCGGGCGACCTTTGGTCGCCCGGCCTCAGTGAGCGAGC
GAGCGCGCAGAGAGGGAGTGGCCAACTCCATCACTAGGGGTTCCT
Exemplary 3′ AAV ITR
(SEQ ID NO: 9)
AGGAACCCCTAGTGATGGAGTTGGCCACTCCCTCTCTGCGCGCTCGCTCG
CTCACTGAGGCCGGGCGACCAAAGGTCGCCCGACGCCCGGGCTTTGCCCG
GGCGGCCTCAGTGAGCGAGCGAGCGCGCAGAGAGGGAGTGGCCAA
Exemplary 5′ AAV ITR
(SEQ ID NO: 52)
CTGCGCGCTCGCTCGCTCACTGAGGCCGCCCGGGCGTCGGGCGACCTTTG
GTCGCCCGGCCTCAGTGAGCGAGCGAGCGCGCAGAGAGGGAGTGGCCAAC
TCCATCACTAGGGGTTCCT
Exemplary 3′ AAV ITR
(SEQ ID NO: 53)
AGGAACCCCTAGTGATGGAGTTGGCCACTCCCTCTCTGCGCGCTCGCTCG
CTCACTGAGGCCGGGCGACCAAAGGTCGCCCGACGCCCGGGCTTTGCCCG
GGCGGCCTCAGTGAGCGAGCGAGCGCGCAG

Promoters

In some aspects, the disclosure is directed to constructs comprising a cell selective promoter which can be used to regulate (e.g., increase) expression of connexin 26 protein in a cell (e.g., an inner ear cell, e.g., a supporting cell). In some aspects, the constructs provide reduced toxicity that may be associated with expression of connexin 26 in some cells (e.g., an inner ear cell, e.g., a hair cell).

In some embodiments, a construct (e.g., an rAAV construct) comprises a promoter. The term “promoter” refers to a DNA sequence recognized by enzymes/proteins that can promote and/or initiate transcription of an operably linked gene (e.g., a GJB2 gene). For example, a promoter typically refers to, e.g., a nucleotide sequence to which an RNA polymerase and/or any associated factor binds and from which it can initiate transcription. Thus, in some embodiments, a construct (e.g., an rAAV construct) comprises a promoter operably linked to one of the non-limiting example promoters described herein.

In some embodiments, a promoter is an inducible promoter, a constitutive promoter, a mammalian cell promoter, a viral promoter, a chimeric promoter, an engineered promoter, a tissue-specific promoter, or any other type of promoter known in the art. In some embodiments, a promoter is a RNA polymerase II promoter, such as a mammalian RNA polymerase II promoter. In some embodiments, a promoter is a RNA polymerase III promoter, including, but not limited to, a HI promoter, a human U6 promoter, a mouse U6 promoter, or a swine U6 promoter. A promoter will generally be one that is able to promote transcription in an inner ear cell. In some embodiments, a promoter is a cochlea-specific promoter or a cochlea-oriented promoter. In some embodiments, a promoter is a hair cell specific promoter, or a supporting cell specific promoter.

A variety of promoters are known in the art, which can be used herein. Non-limiting examples of promoters that can be used herein include: human EF1α, human cytomegalovirus (CMV) (U.S. Pat. No. 5,168,062, which is incorporated in its entirety herein by reference), human ubiquitin C (UBC), mouse phosphoglycerate kinase 1, polyoma adenovirus, simian virus 40 (SV40), β-globin, β-actin, α-fetoprotein, γ-globin, β-interferon, γ-glutamyl transferase, mouse mammary tumor virus (MMTV), Rous sarcoma virus, rat insulin, glyceraldehyde-3-phosphate dehydrogenase, metallothionein II (MT II), amylase, cathepsin, MI muscarinic receptor, retroviral LTR (e.g., human T-cell leukemia virus HTLV), AAV ITR, interleukin-2, collagenase, platelet-derived growth factor, adenovirus 5 E2, stromelysin, murine MX gene, glucose regulated proteins (GRP78 and GRP94), α-2-macroglobulin, vimentin, MHC class I gene H-2K b, HSP70, proliferin, tumor necrosis factor, thyroid stimulating hormone a gene, immunoglobulin light chain, T-cell receptor, HLA DQa and DQ, interleukin-2 receptor, MHC class II, MHC class II HLA-DRa, muscle creatine kinase, prealbumin (transthyretin), elastase I, albumin gene, c-fos, c-HA-ras, neural cell adhesion molecule (NCAM), H2B (TH2B) histone, rat growth hormone, human serum amyloid (SAA), troponin I (TN I), duchenne muscular dystrophy, human immunodeficiency virus, ATOH1, GJB2, SLC26A4, LGR5, SYN1, GFAP, GDF6, IGFBP2, RBP7, GJB6, PARM1, and Gibbon Ape Leukemia Virus (GALV) promoters. Additional examples of promoters are known in the art. See, e.g., Lodish, Molecular Cell Biology, Freeman and Company, New York 2007, each of which is incorporated in its entirety herein by reference. In some embodiments, a promoter is the CMV immediate early promoter. In some embodiments, the promoter is a CBA promoter. In some embodiments, the promoter is a CAG promoter or a CAG/CBA promoter. In some embodiments, the promoter comprises or consists of SEQ ID NO: 10. In some embodiments, a promoter comprises or consists of SEQ ID NO: 11. In certain embodiments, a promoter comprises a CMV/CBA enhancer/promoter construct exemplified in SEQ ID NO: 12. In certain embodiments, a promoter comprises a CMV/CBA enhancer/promoter construct exemplified in SEQ ID NO: 13. In certain embodiments, a promoter comprises a CAG promoter or CMV/CBA/SV-40 enhancer/promoter construct exemplified in SEQ ID NO: 14. In certain embodiments, a promoter comprises a CAG promoter or CMV/CBA/SV-40 enhancer/promoter construct exemplified in SEQ ID NO: 15. In some aspects, a promoter comprises a ATOH1 enhance/promoter construct of SEQ ID NO: 16. In some aspects, a promoter comprises a GJB2 enhance/promoter construct of SEQ ID NO: 17. In some aspects, a promoter comprises a GJB2 enhance/promoter construct of SEQ ID NO: 61. In some aspects, a promoter is an endogenous human SLC26A4 enhancer-promoter sequence comprised within SEQ ID NO: 54. In some aspects, a promoter is an endogenous human LGR5 enhancer-promoter sequence comprised within SEQ ID NO: 55. In some aspects, a promoter is an endogenous human SYN1 enhancer-promoter sequence comprised within SEQ ID NO: 56. In some aspects, a promoter is an endogenous human GFAP enhancer-promoter sequence comprised within SEQ ID NO: 57 or SEQ ID NO: 62. In some aspects, a promoter is an endogenous human IGFBP2 enhancer-promoter sequence comprised within SEQ ID NO: 95. In certain aspects, a promoter is an endogenous human RBP7 promoter as set forth in SEQ ID NO: 98. In certain aspects, a promoter is an endogenous human GJB6 promoter as set forth in SEQ ID NO: 101. In certain aspects, a promoter is an endogenous human PARM1 promoter as set forth in SEQ ID NO: 104

In some aspects, the promoter comprises a GJB6 and a hGJB2 minimal promoter. In some aspects, the GJB6 promoter comprises a nucleic acid sequence with at least 85%, at least 90%, at least 95%, at least 96%, at least 97%, at least 98% at least 99%, or 100% identity to SEQ ID NO: 91 and the hGJB2 minimal promoter comprises a nucleic acid sequence with at least 85%, at least 90%, at least 95%, at least 96%, at least 97%, at least 98% at least 99%, or 100% to SEQ ID NO: 91. In some aspects, the GJB6 has the nucleic acid sequence of SEQ ID NO: 91 and the hGJB2 minimal promoter has the nucleic acid sequence of SEQ ID NO: 91.

In some aspects, the promoter comprises a IGFBP2 promoter and a hGJB2 minimal promoter. In some aspects, the IGFBP2 promoter comprises a nucleic acid sequence with at least 85%, at least 90%, at least 95%, at least 96%, at least 97%, at least 98% at least 99%, or 100% identity to SEQ ID NO: 95 and the hGJB2 minimal promoter comprises a nucleic acid sequence with at least 85%, at least 90%, at least 95%, at least 96%, at least 97%, at least 98% at least 99%, or 100% to SEQ ID NO: 91. In some aspects, the IGFBP2 has the nucleic acid sequence of SEQ ID NO: 95 and the hGJB2 minimal promoter has the nucleic acid sequence of SEQ ID NO: 91.

In some aspects, the promoter comprises a RBP7 promoter and a hGJB2 minimal promoter. In some aspects, the RBP7 promoter comprises a nucleic acid sequence with at least 85%, at least 90%, at least 95%, at least 96%, at least 97%, at least 98% at least 99%, or 100% identity to SEQ ID NO: 98 and the hGJB2 minimal promoter comprises a nucleic acid sequence with at least 85%, at least 90/a, at least 95%, at least 96%, at least 97%, at least 98% at least 99%, or 100% to SEQ ID NO: 91. In some aspects, the RBP7 has the nucleic acid sequence of SEQ ID NO: 98 and the hGJB2 minimal promoter has the nucleic acid sequence of SEQ ID NO: 91.

In some aspects, the promoter comprises a GJB6 promoter and a hGJB2 minimal promoter. In some aspects, the GJB6 promoter comprises a nucleic acid sequence with at least 85%, at least 90%, at least 95%, at least 96%, at least 97%, at least 98% at least 99%, or 100% identity to SEQ ID NO: 101 and the hGJB2 minimal promoter comprises a nucleic acid sequence with at least 85%, at least 90%, at least 95%, at least 96%, at least 97%, at least 98% at least 99%, or 100% to SEQ ID NO: 91. In some aspects, the GJB6 has the nucleic acid sequence of SEQ ID NO: 101 and the hGJB2 minimal promoter has the nucleic acid sequence of SEQ ID NO: 91.

In some aspects, the promoter comprises a PARM1 promoter and a hGJB2 minimal promoter. In some aspects, the PARM1 promoter comprises a nucleic acid sequence with at least 85%, at least 90%, at least 95%, at least 96%, at least 97%, at least 98% at least 99%, or 100% identity to SEQ ID NO: 104 and the hGJB2 minimal promoter comprises a nucleic acid sequence with at least 85%, at least 90%, at least 95%, at least 96%, at least 97%, at least 98% at least 99%, or 100% to SEQ ID NO: 91. In some aspects, the PARM1 has the nucleic acid sequence of SEQ ID NO: 104 and the hGJB2 minimal promoter has the nucleic acid sequence of SEQ ID NO: 91.

In some embodiments, a promoter sequence is at least 85%, at least 90%, at least 95%, at least 96%, at least 97%, at least 98% at least 99%, or 100% identical to the promoter sequences represented by SEQ ID NO: 10. In some aspects, a promoter sequence is at least 85%, at least 90%, at least 95%, at least 96%, at least 97%, at least 98% at least 99%, or 100% identical to the promoter sequences represented by SEQ ID NO: 11. In some aspects, a promoter sequence is at least 85%, at least 90%, at least 95%, at least 96%, at least 97%, at least 98% at least 99%, or 100% identical to the promoter sequences represented by SEQ ID NO: 91. In some aspects, a promoter is an endogenous human GDF6 promoter sequence comprised within SEQ ID NO: 90. In some aspects, an promoter sequence is at least 85%, at least 90%, at least 95%, at least 96%, at least 97%, at least 98% at least 99%, or 100% identical to a promoter sequence represented by SEQ ID NO: 95. In some aspects, an promoter sequence is at least 85%, at least 90%, at least 95%, at least 96%, at least 97%, at least 98% at least 99%, or 100% identical to a promoter sequence represented by SEQ ID NO: 98. In some aspects, an promoter sequence is at least 85%, at least 90%, at least 95%, at least 96%, at least 97%, at least 98% at least 99%, or 100% identical to promoter sequence represented by SEQ ID NO: 101. In some aspects, a promoter sequence is at least 85%, at least 90%, at least 95%, at least 96%, at least 97%, at least 98% at least 99%, or 100% identical to promoter sequence represented by SEQ ID NO: 104.

In some aspects, an enhancer-promoter sequence is at least 85%, at least 90%, at least 95%, at least 96%, at least 97%, at least 98% at least 99%, or 100% identical to enhancer-promoter sequence represented by SEQ ID NO: 12. In some aspects, an enhancer-promoter sequence is at least 85%, at least 90%, at least 95%, at least 96%, at least 97%, at least 98% at least 99%, or 100% identical to enhancer-promoter sequence represented by SEQ ID NO: 13. In some aspects, an enhancer-promoter sequence is at least 85%, at least 90%, at least 95%, at least 96%, at least 97%, at least 98% at least 99%, or 100% identical to enhancer-promoter sequence represented by SEQ ID NO: 14. In some aspects, an enhancer-promoter sequence is at least 85%, at least 90%, at least 95%, at least 96%, at least 97%, at least 98% at least 99%, or 100% identical to enhancer-promoter sequence represented by SEQ ID NO: 15. In some aspects, an enhancer-promoter sequence is at least 85%, at least 90%, at least 95%, at least 96%, at least 97%, at least 98% at least 99%, or 100% identical to enhancer-promoter sequence represented by SEQ ID NO: 16. In some aspects, an enhancer-promoter sequence is at least 85%, at least 90%, at least 95%, at least 96%, at least 97%, at least 98% at least 99%, or 100% identical to enhancer-promoter sequence represented by SEQ ID NO: 17. In some aspects, an enhancer-promoter sequence is at least 85%, at least 90%, at least 95%, at least 96%, at least 97%, at least 98% at least 99%, or 100% identical to enhancer-promoter sequence represented by SEQ ID NO: 61. In some aspects, an enhancer-promoter sequence is at least 85%, at least 90%, at least 95%, at least 96%, at least 97%, at least 98% at least 99%, or 100% identical to enhancer-promoter sequence represented by SEQ ID NO: 54. In some aspects, an enhancer-promoter sequence is at least 85%, at least 90%, at least 95%, at least 96%, at least 97%, at least 98% at least 99%, or 100% identical to enhancer-promoter sequence represented by SEQ ID NO: 55. In some aspects, an enhancer-promoter sequence is at least 85%, at least 90%, at least 95%, at least 96%, at least 97%, at least 98% at least 99%, or 100% identical to enhancer-promoter sequence represented by SEQ ID NO: 56. In some aspects, an enhancer-promoter sequence is at least 85%, at least 90%, at least 95%, at least 96%, at least 97%, at least 98% at least 99%, or 100% identical to enhancer-promoter sequence represented by SEQ ID NO: 57 or SEQ ID NO: 62. In some aspects, an promoter sequence is at least 85%, at least 90%, at least 95%, at least 96%, at least 97%, at least 98% at least 99%, or 100% identical to enhancer-promoter sequence represented by SEQ ID NO: 90.

The term “constitutive” promoter refers to a nucleotide sequence that, when operably linked with a nucleic acid encoding a protein (e.g., a connexin 26 protein), causes RNA to be transcribed from the nucleic acid in a cell under most or all physiological conditions.

Examples of constitutive promoters include, without limitation, the retroviral Rous sarcoma virus (RSV) LTR promoter, the cytomegalovirus (CMV) promoter (see, e.g., Boshart et al., Cell 41:521-530, 1985, which is incorporated in its entirety herein by reference), the SV40 promoter, the dihydrofolate reductase promoter, the beta-actin promoter, the phosphoglycerol kinase (PGK) promoter, and the EFl-alpha promoter (Invitrogen). In some aspects, the promoter is a constitutive promoter. In some aspects, the constitutive promoter is a CAG promoter, a CBA promoter, a CMV promoter, a CMV/CBA enhancer/promoter, or a CB7 promoter. In some aspects, the a CMV/CBA enhancer/promoter comprises a nucleic acid with at least 85%, at least 90%, at least 95%, at least 96%, at least 97%, at least 98% at least 99%, or 100% identity to SEQ ID NOs: 12 or 13. In some aspects, the a CMV/CBA enhancer/promoter comprises a nucleic acid of SEQ ID NO: 12. In some aspects, the a CMV/CBA enhancer/promoter comprises a nucleic acid of SEQ ID NO: 13. In some aspects, the CBA promoter comprises a nucleic acid with at least 85%, at least 90%, at least 95%, at least 96%, at least 97%, at least 98% at least 99%, or 100% identity to SEQ ID NOs: 10 or 11. In some aspects, the CBA promoter comprises a nucleic acid of SEQ ID NO: 10. In some aspects, the CBA promoter comprises a nucleic acid of SEQ ID NO: 11.

In some aspects, the CMV promoter comprises a nucleic acid with at least 85%, at least 90%, at least 95%, at least 96%, at least 97%, at least 98% at least 99%, or 100% identity to SEQ ID NOs: 14 or 15. In some aspects, the CMV promoter comprises a nucleic acid of SEQ ID NO: 14. In some aspects, the CMV promoter comprises a nucleic acid of SEQ ID NO: 15.

Inducible promoters allow regulation of gene expression and can be regulated by exogenously supplied compounds, environmental factors such as temperature, or the presence of a specific physiological state, e.g., acute phase, a particular differentiation state of the cell, or in replicating cells only. Inducible promoters and inducible systems are available from a variety of commercial sources, including, without limitation, Invitrogen, Clontech, and Ariad. Additional examples of inducible promoters are known in the art.

Examples of inducible promoters regulated by exogenously supplied compounds include the zinc-inducible sheep metallothionein (MT) promoter, the dexamethasone (Dex)-inducible mouse mammary tumor virus (MMTV) promoter, the T7 polymerase promoter system (WO 98/10088, which is incorporated in its entirety herein by reference); the ecdysone insect promoter (No et al., Proc. Natl. Acad Sci. US.A. 93:3346-3351, 1996, which is incorporated in its entirety herein by reference), the tetracycline-repressible system (Gossen et al., Proc. Natl. Acad Sci. US.A. 89:5547-5551, 1992, which is incorporated in its entirety herein by reference), the tetracycline-inducible system (Gossen et al., Science 268:1766-1769, 1995, see also Harvey et al., Curr. Opin. Chem. Biol. 2:512-518, 1998, each of which is incorporated in their entirety herein by reference), the RU486-inducible system (Wang et al., Nat. Biotech. 15:239-243, 1997, and Wang et al., Gene Ther. 4:432-441, 1997, each of which is incorporated in their entirety herein by reference), and the rapamycin-inducible system (Magari et al., J Clin. Invest. 100:2865-2872, 1997, which is incorporated in its entirety herein by reference).

The term “tissue-specific” promoter refers to a promoter that is active only in certain specific cell types and/or tissues (e.g., transcription of a specific gene occurs only within cells expressing transcription regulatory and/or control proteins that bind to the tissue-specific promoter).

In some embodiments, regulatory and/or control sequences impart tissue-specific gene expression capabilities. In some cases, tissue-specific regulatory and/or control sequences bind tissue-specific transcription factors that induce transcription in a tissue-specific manner.

In some embodiments, a tissue-specific promoter is a cochlea-specific promoter. In some embodiments, a tissue-specific promoter is a cochlear hair cell-specific promoter. Non-limiting examples of cochlear hair cell-specific promoters include but are not limited to: a ATOH1 promoter, a POU4F3 promoter, a LHX3 promoter, a MYO7A promoter, a MYO6 promoter, a a9ACHR promoter, and a a10ACHR promoter. In some embodiments, a promoter is a cochlear hair cell-specific promoter such as a PRESTIN promoter or an ONCOMOD promoter. See, e.g., Zheng et al., Nature 405:149-155, 2000; Tian et al., Dev. Dyn. 23 1: 199-203, 2004; and Ryan et al., Adv. Otorhinolaryngol. 66: 99-115, 2009, each of which is incorporated in their entirety herein by reference.

In some embodiments, a tissue-specific promoter is an ear cell specific promoter. In some embodiments, a tissue-specific promoter is an inner ear cell specific promoter. In some embodiments, a promoter is a characteristic fragment of a tissue-specific promoter. Non-limiting examples of inner ear non-sensory cell-specific promoters include but are not limited to: GJB2, GJB6, SLC26A4, TECTA, DFNA5, COCH, NDP, SYN1, GFAP, PLP, TAK1, IGFBP2, RBP7, GDF6, PARM1, or SOX21. In some embodiments, a cochlear non-sensory cell specific promoter may be an inner ear supporting cell specific promoter. Non-limiting examples of inner ear supporting cell specific promoters include but are not limited to: SOX2, FGFR3, PROX1, GLAST1, LGR5, HES1, HES5, NOTCH1, JAG1, CDKN1A, CDKN1B, SOX10, P75, CD44, HEY2, LFNG, or S100b.

In some aspects, a cell selective promoter is an ear cell selective promoter. In some aspects, a cell selective promoter is an inner ear cell selective promoter. In some aspects, a promoter is a characteristic fragment of a cell selective promoter. In some aspects, the promoter is a supporting cell selective promoter. In some aspects, the promoter is an inner ear supporting cell selective promoter.

In some aspects, the promoter is a supporting cell selective promoter. In some aspects, the promoter is a hair cell selective promoter. In some aspects, the supporting cells are selected from one or more of inner phalangeal cells/border cells (IPhC), inner pillar cells (IPC), outer pillar cells (OPC), Deiters' cells rows 1 and 2 (DC1/2), Deiters' cells row 3 (DC3), Hensen's cells (Hec), Claudius cells/outer sulcus cells (CC/OSC), interdental cells (Idc), inner sulcus cells (ISC), Kolliker's organ cells (KO), Lateral greater epithelial ridge cells (LGER), and OC90+ cells (OC90).

In some aspects, supporting cell selective promoters are selected from one or more of GJB6, GDF6, PARM1, RBP7, and IGFBP2.

In some aspects, the promoter is an inner ear medial support cell selective promoter. In some aspects, inner ear medial support cells are selected from one or more of lateral greater epithelial ridge cells and inner sulcus cells. In some aspects, inner ear medial support cell selective promoters are selected from one or more of GJB6, IGFBP2, GDF6, PARM1, and GFAP. In some aspects, the promoter is an inner ear sensory epithelial support cell selective promoter. In some aspects, sensory epithelial support cells are selected from one or more of inner pillar cells, outer pillar cells, dieter cells, and inner phalangeal cells. In some aspects, inner ear sensory epithelial support cell selective promoters are selected from one or more of GJB6, IGFBP2, RBP7, GDF6, PARM1, and GFAP.

In some aspects, the inner ear supporting cell selective promoter is a GJB2 promoter. In some aspects, the GJB2 enhance/promoter comprises a nucleic acid sequence with at least 85%, at least 90%, at least 95%, at least 96%, at least 97%, at least 98% at least 99%, or 100% identity to SEQ ID NO: 17. In some aspects, the GJB2 enhance/promoter comprises the nucleic acid sequence of SEQ ID NO: 17. In some aspects, the GJB2 promoter comprises a nucleic acid sequence with at least 85%, at least 90%, at least 95%, at least 96%, at least 97%, at least 98% at least 99%, or 100% identity to SEQ ID NO: 61. In some aspects, the GJB2 promoter comprises the nucleic acid sequence of SEQ ID NO: 61. In some aspects, the GJB2 minimal promoter comprises a nucleic acid sequence with at least 85%, at least 90%, at least 95%, at least 96%, at least 97%, at least 98% at least 99%, or 100% identity to SEQ ID NO: 91. In some aspects, the GJB2 minimal promoter comprises the nucleic acid sequence of SEQ ID NO: 91.

In some aspects, the inner ear supporting cell selective promoter is a GJB6 promoter. In some aspects, the GJB6 promoter comprises a nucleic acid sequence with at least 85%, at least 90%, at least 95%, at least 96%, at least 97%, at least 98% at least 99%, or 100% identity to SEQ ID NO: 101. In some aspects, the GJB6 promoter comprises the nucleic acid sequence of SEQ ID NO: 101.

In some aspects, the inner ear supporting cell selective promoter is a SLC26A4 promoter. In some aspects, the SLC26A4 promoter comprises a nucleic acid sequence with at least 85%, at least 90%, at least 95%, at least 96%, at least 97%, at least 98% at least 99%, or 100% identity to SEQ ID NO: 54. In some aspects, the SLC26A4 promoter comprises the nucleic acid sequence of SEQ ID NO: 54.

In some aspects, the inner ear supporting cell selective promoter is a GFAP promoter. In some aspects, the GFAP promoter comprises a nucleic acid sequence with at least 85%, at least 90%, at least 95%, at least 96%, at least 97%, at least 98% at least 99%, or 100% identity to SEQ ID NO: 57. In some aspects, the GFAP promoter comprises the nucleic acid sequence of SEQ ID NO: 57. In some aspects, the GFAP promoter comprises a nucleic acid sequence with at least 85%, at least 90%, at least 95%, at least 96%, at least 97%, at least 98% at least 99%, or 100% identity to SEQ ID NO: 62. In some aspects, the GFAP promoter comprises the nucleic acid sequence of SEQ ID NO: 62.

In some aspects, the inner ear supporting cell selective promoter is a IGFBP2 promoter. In some aspects, the IGFBP2 promoter comprises a nucleic acid sequence with at least 85%, at least 90%, at least 95%, at least 96%, at least 97%, at least 98% at least 99%, or 100% identity to SEQ ID NO: 95. In some aspects, the IGFBP2 promoter comprises the nucleic acid sequence of SEQ ID NO: 95.

In some aspects, the inner ear supporting cell selective promoter is a RBP7 promoter. In some aspects, the RBP7 promoter comprises a nucleic acid sequence with at least 85%, at least 90%, at least 95%, at least 96%, at least 97%, at least 98% at least 99%, or 100% identity to SEQ ID NO: 98. In some aspects, the RBP7 promoter comprises the nucleic acid sequence of SEQ ID NO: 98.

In some aspects, the inner ear supporting cell selective promoter is a GDF6 promoter. In some aspects, the GDF6 promoter comprises a nucleic acid sequence with at least 85%, at least 90%, at least 95%, at least 96%, at least 97%, at least 98% at least 99%, or 100% identity to SEQ ID NO: 90. In some aspects, the GDF6 promoter comprises the nucleic acid sequence of SEQ ID NO: 90.

In some aspects, the inner ear supporting cell selective promoter is a PARM1 promoter. In some aspects, the PARM1 promoter comprises a nucleic acid sequence with at least 85%, at least 90%, at least 95%, at least 96%, at least 97%, at least 98% at least 99%, or 100% identity to SEQ ID NO: 40. In some aspects, the PARM1 promoter comprises the nucleic acid sequence of SEQ ID NO: 40.

In some aspects, the inner ear supporting cell selective promoter is a LGR5 promoter. In some aspects, the LGR5 promoter comprises a nucleic acid sequence with at least 85%, at least 90%, at least 95%, at least 96%, at least 97%, at least 98% at least 99%, or 100% identity to SEQ ID NO: 55. In some aspects, the LGR5 promoter comprises the nucleic acid sequence of SEQ ID NO: 55.

In some aspects, the inner ear supporting cell selective promoter is a ATOH1 promoter. In some aspects, the ATOH1 promoter comprises a nucleic acid sequence with at least 85%, at least 90%, at least 95%, at least 96%, at least 97%, at least 98% at least 99%, or 100% identity to SEQ ID NO: 16. In some aspects, the ATOH1 promoter comprises the nucleic acid sequence of SEQ ID NO: 16.

In some aspects, the inner ear supporting cell selective promoter comprises a GJB6 and a hGJB2 minimal promoter. In some aspects, the GJB6 promoter comprises a nucleic acid sequence with at least 85%, at least 90%, at least 95%, at least 96%, at least 97%, at least 98% at least 99%, or 100% identity to SEQ ID NO: 91 and the hGJB2 minimal promoter comprises a nucleic acid sequence with at least 85%, at least 90%, at least 95%, at least 96%, at least 97%, at least 98% at least 99%, or 100% to SEQ ID NO: 91. In some aspects, the GJB6 has the nucleic acid sequence of SEQ ID NO: 91 and the hGJB2 minimal promoter has the nucleic acid sequence of SEQ ID NO: 91.

In some aspects, the inner ear supporting cell selective promoter comprises a IGFBP2 promoter and a hGJB2 minimal promoter. In some aspects, the IGFBP2 promoter comprises a nucleic acid sequence with at least 85%, at least 90%, at least 95%, at least 96%, at least 97%, at least 98% at least 99%, or 100% identity to SEQ ID NO: 95 and the hGJB2 minimal promoter comprises a nucleic acid sequence with at least 85%, at least 90%, at least 95%, at least 96%, at least 97%, at least 98% at least 99%, or 100% to SEQ ID NO: 91. In some aspects, the IGFBP2 has the nucleic acid sequence of SEQ ID NO: 95 and the hGJB2 minimal promoter has the nucleic acid sequence of SEQ ID NO: 91.

In some aspects, the inner ear supporting cell selective promoter comprises a RBP7 promoter and a hGJB2 minimal promoter. In some aspects, the RBP7 promoter comprises a nucleic acid sequence with at least 85%, at least 90%, at least 95%, at least 96%, at least 97%, at least 98% at least 99%, or 100% identity to SEQ ID NO: 98 and the hGJB2 minimal promoter comprises a nucleic acid sequence with at least 85%, at least 90%, at least 95%, at least 96%, at least 97%, at least 98% at least 99%, or 100% to SEQ ID NO: 91. In some aspects, the RBP7 has the nucleic acid sequence of SEQ ID NO: 98 and the hGJB2 minimal promoter has the nucleic acid sequence of SEQ ID NO: 91.

In some aspects, the inner ear supporting cell selective promoter comprises a GJB6 promoter and a hGJB2 minimal promoter. In some aspects, the GJB6 promoter comprises a nucleic acid sequence with at least 85%, at least 90%, at least 95%, at least 96%, at least 97%, at least 98% at least 99%, or 100% identity to SEQ ID NO: 101 and the hGJB2 minimal promoter comprises a nucleic acid sequence with at least 85%, at least 90%, at least 95%, at least 96%, at least 97%, at least 98% at least 99%, or 100% to SEQ ID NO: 91. In some aspects, the GJB6 has the nucleic acid sequence of SEQ ID NO: 101 and the hGJB2 minimal promoter has the nucleic acid sequence of SEQ ID NO: 91.

In some aspects, the inner ear supporting cell selective promoter comprises a PARM1 promoter and a hGJB2 minimal promoter. In some aspects, the PARM1 promoter comprises a nucleic acid sequence with at least 85%, at least 90%, at least 95%, at least 96%, at least 97%, at least 98% at least 99%, or 100% identity to SEQ ID NO: 104 and the hGJB2 minimal promoter comprises a nucleic acid sequence with at least 85%, at least 90%, at least 95%, at least 96%, at least 97%, at least 98% at least 99%, or 100% to SEQ ID NO: 91. In some aspects, the PARM1 has the nucleic acid sequence of SEQ ID NO: 104 and the hGJB2 minimal promoter has the nucleic acid sequence of SEQ ID NO: 91.

In some aspects, the inner ear supporting cell selective promoter comprises a GJB6 and a hGJB2 minimal promoter. In some aspects, the GJB6 promoter comprises a nucleic acid sequence with at least 85%, at least 90%, at least 95%, at least 96%, at least 97%, at least 98% at least 99%, or 100% identity to SEQ ID NO: 91 and the hGJB2 minimal promoter comprises a nucleic acid sequence with at least 85%, at least 90%, at least 95%, at least 96%, at least 97%, at least 98% at least 99%, or 100% to SEQ ID NO: 91. In some aspects, the GJB6 has the nucleic acid sequence of SEQ ID NO: 91 and the hGJB2 minimal promoter has the nucleic acid sequence of SEQ ID NO: 91.

In some aspects, the inner ear supporting cell selective promoter comprises a IGFBP2 promoter and a hGJB2 minimal promoter. In some aspects, the IGFBP2 promoter comprises a nucleic acid sequence with at least 85%, at least 90%, at least 95%, at least 96%, at least 97%, at least 98% at least 99%, or 100% identity to SEQ ID NO: 95 and the hGJB2 minimal promoter comprises a nucleic acid sequence with at least 85%, at least 90%, at least 95%, at least 96%, at least 97%, at least 98% at least 99%, or 100% to SEQ ID NO: 91. In some aspects, the IGFBP2 has the nucleic acid sequence of SEQ ID NO: 95 and the hGJB2 minimal promoter has the nucleic acid sequence of SEQ ID NO: 91.

In some aspects, the inner ear supporting cell selective promoter comprises a RBP7 promoter and a hGJB2 minimal promoter. In some aspects, the RBP7 promoter comprises a nucleic acid sequence with at least 85%, at least 90%, at least 95%, at least 96%, at least 97%, at least 98% at least 99%, or 100% identity to SEQ ID NO: 98 and the hGJB2 minimal promoter comprises a nucleic acid sequence with at least 85%, at least 90%, at least 95%, at least 96%, at least 97%, at least 98% at least 99%, or 100% to SEQ ID NO: 91. In some aspects, the RBP7 has the nucleic acid sequence of SEQ ID NO: 98 and the hGJB2 minimal promoter has the nucleic acid sequence of SEQ ID NO: 91.

In some aspects, the inner ear supporting cell selective promoter comprises a GJB6 promoter and a hGJB2 minimal promoter. In some aspects, the GJB6 promoter comprises a nucleic acid sequence with at least 85%, at least 90%, at least 95%, at least 96%, at least 97%, at least 98% at least 99%, or 100% identity to SEQ ID NO: 101 and the hGJB2 minimal promoter comprises a nucleic acid sequence with at least 85%, at least 90%, at least 95%, at least 96%, at least 97%, at least 98% at least 99%, or 100% to SEQ ID NO: 91. In some aspects, the GJB6 has the nucleic acid sequence of SEQ ID NO: 101 and the hGJB2 minimal promoter has the nucleic acid sequence of SEQ ID NO: 91.

In some aspects, the inner ear supporting cell selective promoter comprises a PARM1 promoter and a hGJB2 minimal promoter. In some aspects, the PARM1 promoter comprises a nucleic acid sequence with at least 85%, at least 90%, at least 95%, at least 96%, at least 97%, at least 98% at least 99%, or 100% identity to SEQ ID NO: 104 and the hGJB2 minimal promoter comprises a nucleic acid sequence with at least 85%, at least 90%, at least 95%, at least 96%, at least 97%, at least 98% at least 99%, or 100% to SEQ ID NO: 91. In some aspects, the PARM1 has the nucleic acid sequence of SEQ ID NO: 104 and the hGJB2 minimal promoter has the nucleic acid sequence of SEQ ID NO: 91.

In some embodiments, provided AAV constructs comprise a promoter sequence selected from a CAG, a CBA, a CMV, or a CB7 promoter. In some embodiments of any of the therapeutic compositions described herein, the first or sole AAV construct further includes at least one promoter sequence selected from Cochlea and/or inner ear specific promoters.

Exemplary CBA promoter
(SEQ ID NO: 10)
GTCGAGGTGAGCCCCACGTTCTGCTTCACTCTCCCCATCTCCCCCCCCTCCCCACCCCCAATTTT
GTATTTATTTATTTTTTAATTATTTTGTGCAGCGATGGGGGCGGGGGGGGGGGGGGCGCGCGCCA
GGCGGGGCGGGGCGGGGCGAGGGGCGGGGCGGGGCGAGGCGGAGAGGTGCGGCGGCAGCCAATCA
GAGCGGCGCGCTCCGAAAGTTTCCTTTTATGGCGAGGCGGCGGCGGCGGCGGCCCTATAAAAAGC
GAAGCGCGCGGCGGGCG
Exemplary CBA promoter
(SEQ ID NO: 11)
GTCGAGGTGAGCCCCACGTTCTGCTTCACTCTCCCCATCTCCCCCCCCTCCCCACCCCCAATTTT
GTATTTATTTATTTTTTAATTATTTTGTGCAGCGATGGGGGCGGGGGGGGGGGGGGGGCGCGCGC
CAGGCGGGGCGGGGCGGGGCGAGGGGCGGGGCGGGGCGAGGCGGAGAGGTGCGGCGGCAGCCAAT
CAGAGCGGCGCGCTCCGAAAGTTTCCTTTTATGGCGAGGCGGCGGCGGCGGCGGCCCTATAAAAA
GCGAAGCGCGCGGCGGGCG
Exemplary CMV/CBA enhancer/promoter
(SEQ ID NO: 12)
GACATTGATTATTGACTAGTTATTAATAGTAATCAATTACGGGGTCATTAGTTCATAGCCCATAT
ATGGAGTTCCGCGTTACATAACTTACGGTAAATGGCCCGCCTGGCTGACCGCCCAACGACCCCCG
CCCATTGACGTCAATAATGACGTATGTTCCCATAGTAACGCCAATAGGGACTTTCCATTGACGTC
AATGGGTGGACTATTTACGGTAAACTGCCCACTTGGCAGTACATCAAGTGTATCATATGCCAAGT
ACGCCCCCTATTGACGTCAATGACGGTAAATGGCCCGCCTGGCATTATGCCCAGTACATGACCTT
ATGGGACTTTCCTACTTGGCAGTACATCTACGTATTAGTCATCGCTATTACCATGGGTCGAGGTG
AGCCCCACGTTCTGCTTCACTCTCCCCATCTCCCCCCCCTCCCCACCCCCAATTTTGTATTTATT
TATTTTTTAATTATTTTGTGCAGCGATGGGGGCGGGGGGGGGGGGGGCGCGCGCCAGGCGGGGCG
GGGCGGGGCGAGGGGCGGGGCGGGGCGAGGCGGAGAGGTGCGGCGGCAGCCAATCAGAGCGGCGC
GCTCCGAAAGTTTCCTTTTATGGCGAGGCGGCGGCGGCGGCGGCCCTATAAAAAGCGAAGCGCGC
GGCGGGCG
Exemplary CMV/CBA enhancer/promoter
(SEQ ID NO: 13)
GACATTGATTATTGACTAGTTATTAATAGTAATCAATTACGGGGTCATTAGTTCATAGCCCATAT
ATGGAGTTCCGCGTTACATAACTTACGGTAAATGGCCCGCCTGGCTGACCGCCCAACGACCCCCG
CCCATTGACGTCAATAATGACGTATGTTCCCATAGTAACGCCAATAGGGACTTTCCATTGACGTC
AATGGGTGGACTATTTACGGTAAACTGCCCACTTGGCAGTACATCAAGTGTATCATATGCCAAGT
ACGCCCCCTATTGACGTCAATGACGGTAAATGGCCCGCCTGGCATTATGCCCAGTACATGACCTT
ATGGGACTTTCCTACTTGGCAGTACATCTACGTATTAGTCATCGCTATTACCATGGGTCGAGGTG
AGCCCCACGTTCTGCTTCACTCTCCCCATCTCCCCCCCCTCCCCACCCCCAATTTTGTATTTATT
TATTTTTTAATTATTTTGTGCAGCGATGGGGGCGGGGGGGGGGGGGGGGCGCGCGCCAGGCGGGG
CGGGGCGGGGCGAGGGGCGGGGCGGGGCGAGGCGGAGAGGTGCGGCGGCAGCCAATCAGAGCGGC
GCGCTCCGAAAGTTTCCTTTTATGGCGAGGCGGCGGCGGCGGCGGCCCTATAAAAAGCGAAGCGC
GCGGCGGGCG
Exemplary CAG enhancer/promoter
(SEQ ID NO: 14)
GACATTGATTATTGACTAGTTATTAATAGTAATCAATTACGGGGTCATTAGTTCATAGCCCATAT
ATGGAGTTCCGCGTTACATAACTTACGGTAAATGGCCCGCCTGGCTGACCGCCCAACGACCCCCG
CCCATTGACGTCAATAATGACGTATGTTCCCATAGTAACGCCAATAGGGACTTTCCATTGACGTC
AATGGGTGGACTATTTACGGTAAACTGCCCACTTGGCAGTACATCAAGTGTATCATATGCCAAGT
ACGCCCCCTATTGACGTCAATGACGGTAAATGGCCCGCCTGGCATTATGCCCAGTACATGACCTT
ATGGGACTTTCCTACTTGGCAGTACATCTACGTATTAGTCATCGCTATTACCATGGGTCGAGGTG
AGCCCCACGTTCTGCTTCACTCTCCCCATCTCCCCCCCCTCCCCACCCCCAATTTTGTATTTATT
TATTTTTTAATTATTTTGTGCAGCGATGGGGGCGGGGGGGGGGGGGGCGCGCGCCAGGCGGGGCG
GGGCGGGGCGAGGGGCGGGGCGGGGCGAGGCGGAGAGGTGCGGCGGCAGCCAATCAGAGCGGCGC
GCTCCGAAAGTTTCCTTTTATGGCGAGGCGGCGGCGGCGGCGGCCCTATAAAAAGCGAAGCGCGC
GGCGGGCGGGAGTCGCTGCGTTGCCTTCGCCCCGTGCCCCGCTCCGCGCCGCCTCGCGCCGCCCG
CCCCGGCTCTGACTGACCGCGTTACTCCCACAGGTGAGCGGGCGGGACGGCCCTTCTCCTCCGGG
CTGTAATTAGCGCTTGGTTTAATGACGGCTCGTTTCTTTTCTGTGGCTGCGTGAAAGCCTTAAAG
GGCTCCGGGAGGGCCCTTTGTGCGGGGGGGAGCGGCTCGGGGGGTGCGTGCGTGTGTGTGTGCGT
GGGGAGCGCCGCGTGCGGCCCGCGCTGCCCGGCGGCTGTGAGCGCTGCGGGCGCGGCGCGGGGCT
TTGTGCGCTCCGCGTGTGCGCGAGGGGAGCGCGGCCGGGGGCGGTGCCCCGCGGTGCGGGGGGGC
TGCGAGGGGAACAAAGGCTGCGTGCGGGGTGTGTGCGTGGGGGGGTGAGCAGGGGGTGTGGGCGC
GGCGGTCGGGCTGTAACCCCCCCCTGCACCCCCCTCCCCGAGTTGCTGAGCACGGCCCGGCTTCG
GGTGCGGGGCTCCGTGCGGGGCGTGGCGCGGGGCTCGCCGTGCCGGGCGGGGGGTGGCGGCAGGT
GGGGGTGCCGGGCGGGGCGGGGCCGCCTCGGGCCGGGGAGGGCTCGGGGGAGGGGCGCGGCGGCC
CCCGGAGCGCCGGCGGCTGTCGAGGCGCGGCGAGCCGCAGCCATTGCCTTTTATGGTAATCGTGC
GAGAGGGCGCAGGGACTTCCTTTGTCCCAAATCTGTGCGGAGCCGAAATCTGGGAGGCGCCGCCG
CACCCCCTCTAGCGGGCGCGGGGCGAAGCGGTGCGGCGCCGGCAGGAAGGAAATGGGCGGGGAGG
GCCTTCGTGCGTCGCCGCGCCGCCGTCCCCTTCTCCCTCTCCAGCCTCGGGGCTGTCCGCGGGGG
GACGGCTGCCTTCGGGGGGGACGGGGCAGGGCGGGGTTCGGCTTCTGGCGTGTGACCGGCGGCTC
TAGAGCCTCTGCTAACCATGTTCATGCCTTCTTCTTTTTCCTACAG
Exemplary CAG enhancer/promoter
(SEQ ID NO: 15)
GACATTGATTATTGACTAGTTATTAATAGTAATCAATTACGGGGTCATTAGTTCATAGCCCATAT
ATGGAGTTCCGCGTTACATAACTTACGGTAAATGGCCCGCCTGGCTGACCGCCCAACGACCCCCG
CCCATTGACGTCAATAATGACGTATGTTCCCATAGTAACGCCAATAGGGACTTTCCATTGACGTC
AATGGGTGGACTATTTACGGTAAACTGCCCACTTGGCAGTACATCAAGTGTATCATATGCCAAGT
ACGCCCCCTATTGACGTCAATGACGGTAAATGGCCCGCCTGGCATTATGCCCAGTACATGACCTT
ATGGGACTTTCCTACTTGGCAGTACATCTACGTATTAGTCATCGCTATTACCATGGGTCGAGGTG
AGCCCCACGTTCTGCTTCACTCTCCCCATCTCCCCCCCCTCCCCACCCCCAATTTTGTATTTATT
TATTTTTTAATTATTTTGTGCAGCGATGGGGGCGGGGGGGGGGGGGGGGCGCGCGCCAGGCGGGG
CGGGGCGGGGCGAGGGGCGGGGCGGGGCGAGGCGGAGAGGTGCGGCGGCAGCCAATCAGAGCGGC
GCGCTCCGAAAGTTTCCTTTTATGGCGAGGCGGCGGCGGCGGCGGCCCTATAAAAAGCGAAGCGC
GCGGCGGGCGGGAGTCGCTGCGTTGCCTTCGCCCCGTGCCCCGCTCCGCGCCGCCTCGCGCCGCC
CGCCCCGGCTCTGACTGACCGCGTTACTCCCACAGGTGAGCGGGCGGGACGGCCCTTCTCCTCCG
GGCTGTAATTAGCGCTTGGTTTAATGACGGCTCGTTTCTTTTCTGTGGCTGCGTGAAAGCCTTAA
AGGGCTCCGGGAGGGCCCTTTGTGCGGGGGGGAGCGGCTCGGGGGGTGCGTGCGTGTGTGTGTGC
GTGGGGAGCGCCGCGTGCGGCCCGCGCTGCCCGGCGGCTGTGAGCGCTGCGGGCGCGGCGCGGGG
CTTTGTGCGCTCCGCGTGTGCGCGAGGGGAGCGCGGCCGGGGGCGGTGCCCCGCGGTGCGGGGGG
GCTGCGAGGGGAACAAAGGCTGCGTGCGGGGTGTGTGCGTGGGGGGGTGAGCAGGGGGTGTGGGC
GCGGCGGTCGGGCTGTAACCCCCCCCTGCACCCCCCTCCCCGAGTTGCTGAGCACGGCCCGGCTT
CGGGTGCGGGGCTCCGTGCGGGGCGTGGCGCGGGGCTCGCCGTGCCGGGCGGGGGGTGGCGGCAG
GTGGGGGTGCCGGGCGGGGCGGGGCCGCCTCGGGCCGGGGAGGGCTCGGGGGAGGGGCGCGGCGG
CCCCCGGAGCGCCGGCGGCTGTCGAGGCGCGGCGAGCCGCAGCCATTGCCTTTTATGGTAATCGT
GCGAGAGGGCGCAGGGACTTCCTTTGTCCCAAATCTGTGCGGAGCCGAAATCTGGGAGGCGCCGC
CGCACCCCCTCTAGCGGGCGCGGGGCGAAGCGGTGCGGCGCCGGCAGGAAGGAAATGGGCGGGGA
GGGCCTTCGTGCGTCGCCGCGCCGCCGTCCCCTTCTCCCTCTCCAGCCTCGGGGCTGTCCGCGGG
GGGACGGCTGCCTTCGGGGGGGACGGGGCAGGGCGGGGTTCGGCTTCTGGCGTGTGACCGGCGGC
TCTAGAGCCTCTGCTAACCATGTTCATGCCTTCTTCTTTTTCCTACAG

In certain embodiments, a promoter is an endogenous human ATOH1 enhancer-promoter as set forth in SEQ ID NO: 16. In some embodiments, an enhancer-promoter sequence is at least 85%, at least 90%, at least 95%, at least 96%, at least 97%, at least 98% at least 99%, or 100% identical to enhancer-promoter sequence represented by SEQ ID NO: 16. In some embodiments, a promoter is an endogenous human ATOH1 enhancer-promoter sequence comprised within SEQ ID NO: 16.

Exemplary Human ATOH1 enhancer-promoter
(SEQ ID NO: 16)
CTATGGAGTTTGCATAACAAACGTTTGGCAGCTCGCTCTCTTACACTCCA
TTAACAAGCTGTAACATATAGCTGCAGGTTGCTATAATCTCATTAATATT
TTGGAAACTTGAATATTGAGTATTTCTGAGTGCTCATTCCCCATATGCCA
GCCACTTCTGCCATGCTGACTGGTTCCTTTCTCTCCATTATTAGCAATTA
GCTTCTTACCTTCCAAAGTCAGATCCAAGGTATCCAAGATACTAGCAAAG
GAATCAACTATGTGTGCAAGTTAAGCATGCTTAATATCACCCAAACAAAC
AAAGAGGCAGCATTTCTTAAAGTAATGAAGATAGATAAATCGGGTTAGTC
CTTTGCGACACTGCTGGTGCTTTCTAGAGTTTTATATATTTTAAGCAGCT
TGCTTTATATTCTGTCTTTGCCTCCCACCCCACCAGCACTTTTATTTGTG
GAGGGTTTTGGCTCGCCACACTTTGGGAAACTTATTTGATTTCACGGAGA
GCTGAAGGAAGATCATTTTTGGCAACAGACAAGTTTAAACACGATTTCTA
TGGGACATTGCTAACTGGGGCCCCTAAGGAGAAAGGGGAAACTGAGCGGA
GAATGGGTTAAATCCTTGGAAGCAGGGGAGAGGCAGGGGAGGAGAGAAGT
CGGAGGAGTATAAAGAAAAGGACAGGAACCAAGAAGCGTGGGGGTGGTTT
GCCGTAATGTGAGTGTTTCTTAATTAGAGAACGGTTGACAATAGAGGGTC
TGGCAGAGGCTCCTGGCCGCGGTGCGGAGCGTCTGGAGCGGAGCACGCGC
TGTCAGCTGGTGAGCGCACTCTCCTTTCAGGCAGCTCCCCGGGGAGCTGT
GCGGCCACATTTAACACCATCATCACCCCTCCCCGGCCTCCTCAACCTCG
GCCTCCTCCTCGTCGACAGCCTTCCTTGGCCCCCACCAGCAGAGCTCACA
GTAGCGAGCGTCTCTCGCCGTCTCCCGCACTCGGCCGGGGCCTCTCTCCT
CCCCCAGCTGCGCAGCGGGAGCCGCCACTGCCCACTGCACCTCCCAGCAA
CCAGCCCAGCACGCAAAGAAGCTGCGCAAAGTTAAAGCCAAGCAATGCCA
AGGGGAGGGGAAGCTGGAGGCGGGCTTTGAGTGGCTTCTGGGCGCCTGGC
GGGTCCAGAATCGCCCAGAGCCGCCCGCGGTCGTGCACATCTGACCCGAG
TCAGCTTGGGCACCAGCCGAGAGCCGGCTCCGCACCGCTCCCGCACCCCA
GCCGCCGGGGTGGTGACACACACCGGAGTCGAATTACAGCCCTGCAATTA
ACATATGAATCTGACGAATTTAAAAGAAGGAAAAAAAAAAAAAAACCTGA
GCAGGCTTGGGAGTCCTCTGCACACAAGAACTTTTCTCGGGGTGTAAAAA
CTCTTTGATTGGCTGCTCGCACGCGCCTGCCCGCGCCCTCCATTGGCTGA
GAAGACACGCGACCGGCGCGAGGAGGGGGTTGGGAGAGGAGCGGGGGGAG
ACTGAGTGGCGCGTGCCGCTTTTTAAAGGGGCGCAGCGCCTTCAGCAACC
GGAGAAGCATAGTTGCACGCGACCTGGTGTGTGATCTCCGAGTGGGTGGG
GGAGGGTCGAGGAGGGAAAAAAAAATAAGACGTTGCAGAAGAGACCCGGA
AAGGGCCTTTTTTTTGGTTGAGCTGGTGTCCCAGTGCTGCCTCCGATCCT
GAGCCTCCGAGCCTTTGCAGTGCAA

In certain embodiments, a promoter is an endogenous human GJB2 enhancer-promoter as set forth in SEQ ID NO: 17, or SEQ ID NO: 61. In some embodiments, an enhancer-promoter sequence is at least 85%, at least 90%, at least 95%, at least 96%, at least 97%, at least 98% at least 99%, or 100% identical to enhancer-promoter sequence represented by SEQ ID NO: 17 or SEQ ID NO: 61. In some embodiments, a promoter is an endogenous human GJB2 enhancer-promoter sequence comprised within SEQ ID NO: 61. In some aspects, a promoter is GJB2 minimal promoter of SEQ ID NO: 91. In some aspects, a promoter is at least 85%, at least 90%, at least 95%, at least 96%, at least 97%, at least 98% at least 99%, or 100% identical to SEQ ID NO: 91.

Exemplary Human GJB2 enhancer-promoter
(SEQ ID NO: 17)
AAGCTTCGGTGAATTTAAAACGTTTGGTGGCAGTGGGTCAAGTAGCCAGG
CGGCTGCGCTAGAGTACCCCGAAGGGACATCGGCGACACCACAAACCTCG
CGCTGGCGGCTCGCCCGCGCCTTTTTCCCCTCCCGCGCGCGCCCGGCCCC
ACTCGCACCCCGGGCGGTGCCATCGCGTCCACTTCCCCGGCCGCCCCATT
CCAGCTCCGGAGCTCGGCCGCAGAAACGCCCGCTCCAGAAGGCGGCCCCC
GCCCCCCGGCCCAAGGACGTGTGTTGGTCCAGCCCCCCGGTTCCCCGAGA
CCCACGCGGCCGGGCAACCGCTCTGGGTCTCGCGGTCCCTCCCCGCGCCA
GGTTCCTGGCCGGGCAGTCCGGGGCCGGCGGGCTCACCTGCGTCGGGAGG
AAGCGCGGCGGGGCCGGGGCGGGGGTCTCGGCGTTGGGGTCTCTGCGCTG
GGGCTCCTGCGCTCCTAGGCGGGTCCTGGGCCGGGCGCCGCCGAGGGGCT
CCGAGTCGGGGAGAGGAGCGCGCGGGCGCTGCGGGGCCGCAACACCTGTC
TCCCGCCGTGGCGCCTTTTAACCGCACCCCACACCCCGCCTCTTCCCTCG
GAGACTGGGAAAGTTACGGAGGGGGCGGCGCCGCGGGCGGAGCGCGCCCG
GCCTCTGGGTCCTCAGAGCTTCCCGGGTCCGCGAACCCCCGACCGCCCCC
GAAAGCCCCGAACCCCCCAAGTCCCCTTCGAGGTCCCGATCTCCTAGTTC
CTTTGAGCCCCCATGAGTTCCCCAAGTGCCCCCAGCGCCCTGAGTCTCCC
CCGGTTACCCCGAGCGCCGCCTCCCCCAGCCCCTTGGCGGCCCGGGTGAA
GCGGGGGCGGCTGAGAGTCGGGACCCCCCAGGAAGCGGCGCCCCAGACCC
CGGCTCCGGCGCTGTGCCGTGGGCGGGGTTCAGGGATGGCTGTGGTCGTT
GTCCTCTGTACTCCGCATAGTGCGAGAGGACTTGGCATTTATGAGCGCTT
CTTTAATTTTTTATTGTTAGAGAAACAGGCATTCCTCCAAGGACTGAAGA
TCTGTTCGAGTCGCGGAGGCTGCGCGGGCCCGCGAGGCTCTCGCAGGGGG
ACCTAGGCTGGGTGGCGGGGCAGTGCCCTCTGGAATGGGGGTTAACGGTG
GCCGAGGAGGGGGCGCCGCTGGTGCCGGCGAAGTCCCCGCTTCTTTCTCC
CCTCAAAATCTCACCAATCCGAACGAACGCCTTCTCGAATTTCCGATTTT
ATTCAATTACTTTCAACAATGTGCCAAGGACTAAGGTTGGGGGCGGTGGG
AGAGACAAGCCTCGTTTTTGCCATGGCCGGCAGGGGGGTCCCGCCATCTG
CGGAGGGTGCCCCCCGCGGCCCCCGGCCCAGCCAACTTCCTCCTCTTTTC
GCAACTGGGGAACTGCAAGGAGGTGACTCCTTTCGGGGTGAGGAGGCCCA
GACTTTTCAGAAAGGAAAGAGGGCAGGTAAAACCTGCCAAGCCCCTTCCT
GCTCGATGCACACAGCACGAAAGGGGGAAACTGATAGGATTCTGCGGAAG
CTT
Exemplary Human GJB2 promoter
(SEQ ID NO: 61)
AAGCTTCCGCAGAATCCTATCAGTTTCCCCCTTTCGTGCTGTGTGCATCG
AGCAGGAAGGGGCTTGGCAGGTTTTACCTGCCCTCTTTCCTTTCTGAAAA
GTCTGGGCCTCCTCACCCCGAAAGGAGTCACCTCCTTGCAGTTCCCCAGT
TGCGAAAAGAGGAGGAAGTTGGCTGGGCCGGGGGCCGCGGGGGGCACCCT
CCGCAGATGGCGGGACCCCCCTGCCGGCCATGGCAAAAACGAGGCTTGTC
TCTCCCACCGCCCCCAACCTTAGTCCTTGGCACATTGTTGAAAGTAATTG
AATAAATCGGAAATTCGAGAAGGCGTTCGTTCGGATTGGTGAGATTTTGA
GGGGAGAAAGAAGCGGGGACTTCGCCGGCACCAGCGGCGCCCCCTCCTCG
GCCACCGTTAACCCCCATTCCAGAGGGCACTGCCCCGCCACCCAGCCTAG
GTCCCCCTGCGAGAGCCTCGCGGGCCCGCGCAGCCTCCGCGACTCGAACA
GATCTTCAGTCCTTGGAGGAATGCCTGTTTCTCTAACAATAAAAAATTAA
AGAAGCGCTCATAAATGCCAAGTCCTCTCGCACTATGCGGAGTACAGAGG
ACAACGACCACAGCCATCCCTGAACCCCGCCCACGGCACAGCGCCGGAGC
CGGGGTCTGGGGCGCCGCTTCCTGGGGGGTCCCGACTCTCAGCCGCCCCC
GCTTCACCCGGGCCGCCAAGGGGCTGGGGGAGGCGGCGCTCGGGGTAACC
GGGGGAGACTCAGGGCGCTGGGGGCACTTGGGGAACTCATGGGGGCTCAA
AGGAACTAGGAGATCGGGACCTCGAAGGGGACTTGGGGGGTTCGGGGCTT
TCGGGGGCGGTCGGGGGTTCGCGGACCCGGGAAGCTCTGAGGACCCAGAG
GCCGGGCGCGCTCCGCCCGCGGCGCCGCCCCCTCCGTAACTTTCCCAGTC
TCCGAGGGAAGAGGCGGGGTGTGGGGTGCGGTTAAAAGGCGCCACGGCGG
GAGACAGGT
Exemplary Human GJB2 minimal promoter
(SEQ ID NO: 91)
AAGCTCTGAGGACCCAGAGGCCGGGCGCGCTCCGCCCGCGGCGCCGCCCC
CTCCGTAACTTTCCCAGTCTCCGAGGGAAGAGGCGGGGTGTGGGGTGCGG
TTAAAAGGCGCCACGGCGGGAGACAGGT

In certain embodiments, a promoter is an endogenous human SLC26A4 enhancer-promoter as set forth in SEQ ID NO: 54. In some embodiments, an enhancer-promoter sequence is at least 85%, at least 90%, at least 95%, at least 96%, at least 97%, at least 98% at least 99%, or 100% identical to enhancer-promoter sequence represented by SEQ ID NO: 54. In some embodiments, a promoter is an endogenous human SLC26A4 enhancer-promoter sequence comprised within SEQ ID NO: 54.

Exemplary Human SLC26A4 enhancer-promoter
(SEQ ID NO: 54)
CGGAAGGTTGATGTACAGAGGTCTGTATTTTGGAGCCTCTTCTGTATTTA
CTTCAGAACACTAACAATCAGGCGAGAATGTTCTGGTTTATCAAACCCTT
CCTTCTGCCTTTCATCTTAACCATGCATTAGTTTTAACAAAGTTCATCCC
AACAGAAGACAAAACACTGATGAGGTAGGATAGCTCCAGCTCCTCCTCCC
TCTCTTCTAGTCTTGATTTCCATGTAGTCCAGTTTATTCCTTCCCTGATT
GTCCAGGAGAATGAGAAAAAGAAAAAACAGAGTCTAGTGGGTAAGAAAGG
GCCACCTGGACGGCTTGATTTGGATTGTGAAATAAAACACACACACATGC
ACACGTAGAATAAGTGGCTAAAATCTGAGTAAATCGTGAACTCTCTGTAT
CCTCCACCCATTGAATACTCCTAAAAGACTTTCTAGAAATTCAAGGACTT
ATTAATATAGAAACCTGGCCATTGTTCCTCTTCTCCTCCCCATGTGGTAT
GAGAGCACCTGTGGCAGGCTCCCAGAGACCACGGACCTCTTCCTCTAGGC
GGGCTCTGCTCTTCTTTAAGGAGTCCCACAGGGCCTGGCCCGCCCCTGAC
CTCGCAACCCTTGAGATTAGTAACGGGATGAGTGAGGATCCGGGTGGCCC
CTGCGTGGCAGCCAGTAAGAGTCTCAGCCTTCCCGGTTCGGGAAAGGGGA
AGAATGCAGGAGGGGTAGGATTTCTTTCCTGATAGGATCGGTTGGGAAAG
ACCGCAGCCTGTGTGTGTCTTTCCCTTCGACCAAGGTGTCTGTTGCTCCG
TAAATAAAACGTCCCACTGCCTTCTGAGAGCGCTATAAAGGCAGCGGAAG
GGTAGTCCGCGGGGCATTCCGGGCGGGGCGCGAGCAGAGACAGGTGAGTT

In certain embodiments, a promoter is an endogenous human LGR5 enhancer-promoter as set forth in SEQ ID NO: 55. In some embodiments, an enhancer-promoter sequence is at least 85%, at least 90%, at least 95%, at least 96%, at least 97%, at least 98% at least 99%, or 100% identical to enhancer-promoter sequence represented by SEQ ID NO: 55. In some embodiments, a promoter is an endogenous human LGR5 enhancer-promoter sequence comprised within SEQ ID NO: 55.

Exemplary Human LGR5 enhancer-promoter
(SEQ ID NO: 55)
AGGGCTATTTGTACCTCAACGAGGGCTTCTCTCCAAGAAAGCCCTGAATC
CTTTTCCTCCTTTTTCCTGCAGATTCACTATAGGACACTTTTTGAAGCAA
GAGCATGCATTTTCCCCCTGGCGCTCTGCAGCGGTTCTCAGAGCCCAGTG
TCACTCACATAGGTGGGACTGCTCTCAGTTCAGAGAGCGCTGGGACACTT
AAGATGAAAAGTCCCTGGAAGTTAGCAAACAGCCATCTGTCACTCTGGCA
TCGATTTACTAAAAGTGACTTCTAGGGTATTCTAAACCACTTTTAAAAAA
CAAATGAGTCACTTCGACTTCCTCACCCCGCAAGAGATAGGAAGGCAGCA
GTGGAGTGCTCGCTCAGGAGCTGTATTTGTTTAGCGATTAGCCTAGAGCT
TTGATTTTAGGGCAAAAGCGAGCCAGACAGTGCGGCAGACGTAAGGATCA
AAAAGGCCACCTATCATTCGCCGGGGACGCCTGCCTCCTTACCCTGATAA
CGTAACTATTTCTCTGCATAGGATTTTAGTTTTTGTGTTTTTGTTTTGTT
TTATTCTGTTTAATCACTTCAAGTATCTCATCCATTATTTGAAGCGGGCT
CGGAGGAAACGTGCCGCATCCTCCAGTCCTTGTGCGTCTGTTTAGGTCTC
TCCGAAGCAGGTCCCTCTCGACTCTTAGATCTGGGTCTCCAGCACGCATG
AAGGGGTAAGGGTGGGGGGGTCCCCTATTCCGGCGCGCGGCGTTGAGCAC
TGAATCTTCCAGGCGGAGGCTCAGTGGGAGCGCCGAGAACTCGCCAGTAC
CGCGCGCTGCCTGCTGCCTGCTGCCTCCCAGCCCAGGACTTGGGAAAGGA
GGGAGGGGACAAGTGGAGGGAAAGTGGGGCCGGGCGGGGGGTGCCTGGGA
AGCCAGGCTGCGCTGACGTCACTGGGCGCGCAATTCGGGCTGGAGCGCTT
TAAAAAACGAGCGTGCAAGCAGAGATGCTGCTCCACACCGCTCAGGCCGC
GAGCAGCAGCAAGGCGCACCGCCACTGTCGCCGCTGCAGCCAGGGCTGCT
CCGAAGGCCGGCGTGGCGGCAACCGGCACCTCTGTCCCCGCCGCGCTTCT
CCTCGCCGCCCACGCCGTGGGGTCAGGAACGCGGCGTCTGGCGCTGCAGA
CGCCCGCTGAGTTGCAGAAGCCCACGGAGCGGCGCCCGGCGCGCCACGGC
CCGTAGCAGTCCGGTGCTGCTCTCCGCCCGCGTCCGGCTCGTGGCCCCCT
ACTTCGGGCACCGACCGGT

In certain embodiments, a promoter is an endogenous human SYN1 enhancer-promoter as set forth in SEQ ID NO: 56. In some embodiments, an enhancer-promoter sequence is at least 85%, at least 90%, at least 95%, at least 96%, at least 97%, at least 98% at least 99%, or 100% identical to enhancer-promoter sequence represented by SEQ ID NO: 56. In some embodiments, a promoter is an endogenous human SYN1 enhancer-promoter sequence comprised within SEQ ID NO: 56.

Exemplary Human SYN1 enhancer-promoter
(SEQ ID NO: 56)
TGCGTATGAGTGCAAGTGGGTTTTAGGACCAGGATGAGGCGGGGTGGGGG
TGCCTACCTGACGACCGACCCCGACCCACTGGACAAGCACCCAACCCCCA
TTCCCCAAATTGCGCATCCCCTATCAGAGAGGGGGAGGGGAAACAGGATG
CGGCGAGGCGCGTGCGCACTGCCAGCTTCAGCACCGCGGACAGTGCCTTC
GCCCCCGCCTGGCGGCGCGCGCCACCGCCGCCTCAGCACTGAAGGCGCGC
TGACGTCACTCGCCGGTCCCCCGCAAACTCCCCTTCCCGGCCACCTTGGT
CGCGTCCGCGCCGCCGCCGGCCCAGCCGGACCGCACCACGCGAGGCGCGA
GATAGGGGGGCACGGGCGCGACCATCTGCGCTGCGGCGCCGGCGACTCAG
CGCTGCCTCAGTCTGCGGTGGGCAGCGGAGGAGTCGTGTCGTGCCTGAGA
GCGCAGTCGAGAA

In certain embodiments, a promoter is an endogenous human GFAP enhancer-promoter as set forth in SEQ ID NO: 57, or SEQ ID NO: 62. In some embodiments, an enhancer-promoter sequence is at least 85%, at least 90%, at least 95%, at least 96%, at least 97%, at least 98% at least 99%, or 100% identical to enhancer-promoter sequence represented by SEQ ID NO: 57 or SEQ ID NO: 62. In some embodiments, a promoter is an endogenous human GFAP enhancer-promoter sequence comprised within SEQ ID NO: 57 or SEQ ID NO: 62.

Exemplary Human GFAP enhancer-promoter
(SEQ ID NO: 57)
CCCACCTCCCTCTCTGTGCTGGGACTCACAGAGGGAGACCTCAGGAGGCAGTCTGTCCATCACAT
GTCCAAATGCAGAGCATACCCTGGGCTGGGCGCAGTGGCGCACAACTGTAATTCCAGCACTTTGG
GAGGCTGATGTGGAAGGATCACTTGAGCCCAGAAGTTCTAGACCAGCCTGGGCAACATGGCAAGA
CCCTATCTCTACAAAAAAAGTTAAAAAATCAGCCACGTGTGGTGACACACACCTGTAGTCCCAGC
TATTCAGGAGGCTGAGGTGAGGGGATCACTTAAGGCTGGGAGGTTGAGGCTGCAGTGAGTCGTGG
TTGCGCCACTGCACTCCAGCCTGGGCAACAGTGAGACCCTGTCTCAAAAGACAAAAAAAAAAAAA
AAAAAAAAAAGAACATATCCTGGTGTGGAGTAGGGGACGCTGCTCTGACAGAGGCTCGGGGGCCT
GAGCTGGCTCTGTGAGCTGGGGAGGAGGCAGACAGCCAGGCCTTGTCTGCAAGCAGACCTGGCAG
CATTGGGCTGGCCGCCCCCCAGGGCCTCCTCTTCATGCCCAGTGAATGACTCACCTTGGCACAGA
CACAATGTTCGGGGTGGGCACAGTGCCTGCTTCCCGCCGCACCCCAGCCCCCCTCAAATGCCTTC
CGAGAAGCCCATTGAGCAGGGGGCTTGCATTGCACCCCAGCCTGACAGCCTGGCATCTTGGGATA
AAAGCAGCACAGCCCCCTAGGGGCTGCCCTTGCTGTGTGGCGCCACCGGCGGTGGAGAACAAGGC
TCTATTCAGCCTGTGCCCAGGAAAGGGGATCAGGGGATGCCCAGGCATGGACAGTGGGTGGCAGG
GGGGGAGAGGAGGGCTGTCTGCTTCCCAGAAGTCCAAGGACACAAATGGGTGAGGGGACTGGGCA
GGGTTCTGACCCTGTGGGACCAGAGTGGAGGGCGTAGATGGACCTGAAGTCTCCAGGGACAACAG
GGCCCAGGTCTCAGGCTCCTAGTTGGGCCCAGTGGCTCCAGCGTTTCCAAACCCATCCATCCCCA
GAGGTTCTTCCCATCTCTCCAGGCTGATGTGTGGGAACTCGAGGAAATAAATCTCCAGTGGGAGA
CGGAGGGGTGGCCAGGGAAACGGGGCGCTGCAGGAATAAAGACGAGCCAGCACAGCCAGCTCATG
TGTAACGGCTTTGTGGAGCTGTCAAGGCCTGGTCTCTGGGAGAGAGGCACAGGGAGGCCAGACAA
GGAAGGGGTGACCTGGAGGGACAGATCCAGGGGCTAAAGTCCTGATAAGGCAAGAGAGTGCCGGC
CCCCTCTTGCCCTATCAGGACCTCCACTGCCACATAGAGGCCATGATTGACCCTTAGACAAAGGG
CTGGTGTCCAATCCCAGCCCCCAGCCCCAGAACTCCAGGGAATGAATGGGCAGAGAGCAGGAATG
TGGGACATCTGTGTTCAAGGGAAGGACTCCAGGAGTCTGCTGGGAATGAGGCCTAGTAGGAAATG
AGGTGGCCCTTGAGGGTACAGAACAGGTTCATTCTTCGCCAAATTCCCAGCACCTTGCAGGCACT
TACAGCTGAGTGAGATAATGCCTGGGTTATGAAATCAAAAAGTTGGAAAGCAGGTCAGAGGTCAT
CTGGTACAGCCCTTCCTTCCCTTTTTTTTTTTTTTTTTTGTGAGACAAGGTCTCTCTCTGTTGCC
CAGGCTGGAGTGGCGCAAACACAGCTCACTGCAGCCTCAACCTACTGGGCTCAAGCAATCCTCCA
GCCTCAGCCTCCCAAAGTGCTGGGATTACAAGCATGAGCCACCCCACTCAGCCCTTTCCTTCCTT
TTTAATTGATGCATAATAATTGTAAGTATTCATCATGGTCCAACCAACCCTTTCTTGACCCACCT
TCCTAGAGAGAGGGTCCTCTTGCTTCAGCGGTCAGGGCCCCAGACCCATGGTCTGGCTCCAGGTA
CCACCTGCCTCATGCAGGAGTTGGCGTGCCCAGGAAGCTCTGCCTCTGGGCACAGTGACCTCAGT
GGGGTGAGGGGAGCTCTCCCCATAGCTGGGCTGCGGCCCAACCCCACCCCCTCAGGCTATGCCAG
GGGGTGTTGCCAGGGGCACCCGGGCATCGCCAGTCTAGCCCACTCCTTCATAAAGCCCTCGCATC
CCAGGAGCGAGCAGAGCCAGAGCAGGTTGGAGAGGAGACGCATCACCTCCGCTGCTCGC
Exemplary Human GFAP enhancer-promoter
(SEQ ID NO: 62)
GAACATATCCTGGTGTGGAGTAGGGGACGCTGCTCTGACAGAGGCTCGGGGGCCTGAGCTGGCTC
TGTGAGCTGGGGAGGAGGCAGACAGCCAGGCCTTGTCTGCAAGCAGACCTGGCAGCATTGGGCTG
GCCGCCCCCCAGGGCCTCCTCTTCATGCCCAGTGAATGACTCACCTTGGCACAGACACAATGTTC
GGGGTGGGCACAGTGCCTGCTTCCCGCCGCACCCCAGCCCCCCTCAAATGCCTTCCGAGAAGCCC
ATTGAGCAGGGGGCTTGCATTGCACCCCAGCCTGACAGCCTGGCATCTTGGGATAAAAGCAGCAC
AGCCCCCTAGGGGCTGCCCTTGCTGTGTGGCGCCACCGGCGGTGGAGAACAAGGCTCTATTCAGC
CTGTGCCCAGGAAAGGGGATCAGGGGATGCCCAGGCATGGACAGTGGGTGGCAGGGGGGGAGAGG
AGGGCTGTCTGCTTCCCAGAAGTCCAAGGACACAAATGGGTGAGGGGAGCTCTCCCCATAGCTGG
GCTGCGGCCCAACCCCACCCCCTCAGGCTATGCCAGGGGGTGTTGCCAGGGGCACCCGGGCATCG
CCAGTCTAGCCCACTCCTTCATAAAGCCCTCGCATCCCAGGAGCGAGCAGAGCCAGAGCAGGTTG
GAGAGGAGACGCATCACCTCCGCTGCTCGC

In certain aspects, a promoter is an endogenous human GDF6 promoter as set forth in SEQ ID NO: 90. In some aspects, an promoter sequence is at least 85%, at least 90%, at least 95%, at least 96%, at least 97%, at least 98% at least 99%, or 100% identical to a promoter sequence represented by SEQ ID NO: 90. In some aspects, a promoter is an endogenous human GDF6 promoter sequence comprised within SEQ ID NO: 90.

Exemplary Human GDF6 promoter
(SEQ ID NO: 90)
CCACAGGTAACTCCGTCGGCGTCCACAGGGGGGCAGGAGATACCATACTG
CACAGTTGTACGTCTTCCATCTGTTTGGTGTAGAAAAATCTAACCACTAC
AAGAATGCCACGGGCACTGTGGCAGACAGAAGCAGCGCTACGCCGCATCG
CCTTTCAGCGTGCAGGCCCAGGAATGAGCGAGGCAGTGGGCGGGGAAGAC
AGGCACGGGGAATCTGGGGACAGATAAAGGAAACTCGTGATGGGGCGAGG
CTGGGCTGAAGAGAAACAGATTGGGGTAGAGCTGCAAAGGGAGGGGTCCA
CTGGAAGGCGAGGGGGGAGGCCGGGAAGAGAGAGGGTGGGAAGGCAGTGT
GAGATGGGAGGGCAGTGTGAGAAGAAAAGCAGGCTGGGGAAGAGGGATTG
GAATGCAGAAGGAACTTGGGGAAGGAGGAAGTCCTGCAGGCGGGAGGGAA
AGAAGAGAGGGGGAGCAGCTAAAGTCTGCGTCAGAAGAGGTTGGGGACTG
CGAGAGGAGAGGCTGGGGCCTGCAGGGGAGCGCAGCAGCTTTTAGCATCG
ATCCAAACTCTAAAGACTCGTGGCCTTTGCCTGACCTCGAGGGTCGGGAA
TAGACGCCTGTCTTTGTGGAGAGCGATACCCAACCGAGAAAATGGGGCTG
TTCCGAGCTGGGCCCTGCGCCTGGCCCAGGGCGAGGCTTCTCTGGCTCCG
GGCTGGCCCCTGAGGGGCAGCACGCAGCCTGCAGCAGAGGCGCCTGCTCC
AAGCTGTCTCTTGGGGGCGCCGCCGCCGCTTCCCTCCTCCGGGGCCGCTC
GCTCCCAGGAAAGTGGAGGCGGCTGGCGAGGACCGAGAGCCGGGGCCGCG
CTGCGGAGGGACCACACCTCCGGGAGTTCGAGGGGGACCCTGGCGCGGCG
GGCCAGCCTTTCGGGCCGGCAGCGCCCGCCTTCCCCCGGTCAGCGCTTGC
GGCCCGCGCCGCGCGCACCGCCCGGCAACCCCGCGCGCGTCCCGCGGGGG
CGCTGCGTCTTCCTGCCACACCGGCGCACCGCGGCCCCTCTCCCCCACAC
CTCCGGCCCGCACCACCCGGCTCTCCTCCCACCCTCCCCACCCCTCCTCT
GCCCTCCCTCCCCATTCCTCCCCTCCCGGCGAGGGGCGGGAGGGGGCGTG
GCGGGGCCGGGGTTTGTGTGGCTGGGACCCGGCTCCTC

In certain aspects, a promoter is an endogenous human IGFBP2 promoter as set forth in SEQ ID NO: 95. In some aspects, an promoter sequence is at least 85%, at least 90%, at least 95%, at least 96%, at least 97%, at least 98% at least 99%, or 100% identical to a promoter sequence represented by SEQ ID NO: 95. In some aspects, a promoter is an endogenous human IGFBP2 enhancer-promoter sequence comprised within SEQ ID NO: 95.

Exemplary Human IGFBP2 promoter
(SEQ ID NO: 95)
AAGAAACTTGCCCGAGTTTACACAGCTAGTAAATGGTTGCATTAGTCAGG
ACAGCTAGCCTATATTACAATAACAACCCTCTCAAATCCTAATGGCTTAA
AACAACAGAGGTTTAATTTATACTCATTAGCTGTTCAAGGCAGGAGGCTC
TATTCTCTAATCCATACAGTCACTCAGGATCCAGGCTGGTGGAGACCCTG
CCATATTGTAGCCTCACCATTTAAAACATGAAGAAGATAGAAAGTGAGGA
GTCATGTAGGTTTTGTTCCGTTGCCTCAGGCTAGGAGTGACAGGTCACTT
CATCTCACTCACAGCTCACTGCCCACAACTAGTCACTTGTGACTGTGCGA
GTTAAGCTTCTGTGTGTGAAGGAAGGAAAAGAGAATGGGATAAAGGTGAA
CATCAGCAGGCTCTACCACAGTAGTTTGAACCAAGACTTGAGCCTAGGTC
ATGTGGCTTCAGAATCTTTGCTCTTAATCACACTAAACAGCCTCTGTAAG
TCATCTTTCCTTCATCCAGTGCCTAAGAACATGCAGTCCAATGCCCTCAT
CCTTCAGAAGAACTTGAGTGAACTCAGAGAAATTGAGTAGAGTGCCACAG
CATGCCCAAGGCCACACACCCTGAGGTTGGCAGTAGGTCCTGAGTTAGAG
TTGTCATTTCTTGGCTCCCCTGGTAGTAGTGGAAAGGTAAGGTTTTGACA
TACTAGTTGGATGACCACGGGCAGGTCACTTAAATTGTCTAAGCATCGTT
TGACCCTTGTAAGAATTAAATGAAATAGCACCTGTAAAAGTGTCTGCACG
GACTTACTGCTGTTAGTTTTGTTCCTTTCTTCCTGTTGTCACTGCACTTC
CCTGCCTGTTACCCAGGCCATGCAGACCAGCCAGGCCTTCGACTTACAGT
GCGGATAAGATTCCAAATCTCCACGGCTGGTTTCCATGCTTTCTTCCAGG
CTTCTGAGGACCCTGTGCTCTGGTTTCTTCTATTTCTTTTCTATTACTTT
TCTGTTACTCTTGAGCACACTTGCTGGAAGCAATATGCATCCAGTTCTCC
CTCTCTTGCCTCATTACACTTTGCAGAACAACTCCAATCCCTTCCAACCA
AGTAGTCCCTTTGAATTTCTTGTCACCCAAGGAATCTCTCTGACAGGGGT
CTTTGTTAGGGTCACACCCCAGGAGATGGTTGATTATGGCTGAGTCCAGC
CTGGAATGATGGGGGTTGGGGGCAGCTTGGGTAGATGACTCAGTAAATCA
AACAGAACAATGAAAGGAGGTCATGCTTGTCCATCTGCATTATTGAAGAC
AGCCATAAATGGCCTTACCCCAGAGCGGGTCTGTCACACCTGGAGAGCTG
ATCTGACCTCTCCAAGACCCCTGCAACTGAGTGTTCTGGGATCTGTCCTG
CAACAAGTGCCTCGAGATTTGTAGGTGGGGGCCCAGAGGGAGGGGGTCTG
CAGACGAAGGGGGCAGGTTTTGCGGGGCACTTAGGGTTCTCATAGGTTGT
AGTCACGAGCTCC

In certain aspects, a promoter is an endogenous human RBP7 promoter as set forth in SEQ ID NO: 98. In some aspects, an promoter sequence is at least 85%, at least 90%, at least 95%, at least 96%, at least 97%, at least 98% at least 99%, or 100% identical to a promoter sequence represented by SEQ ID NO: 98. In some aspects, a promoter is an endogenous human RBP7 enhancer-promoter sequence comprised within SEQ ID NO: 98.

Exemplary Human RBP7 promoter
(SEQ ID NO: 98)
CCCATGGCTCTGTTAAAATCAAAGAAACATCTTTTCCAACAGCCCTTTCA
AACTCCTCATCGCATCTCACTGGCTGATTCAGTCATTTAAACCTGCTTCT
CCCTAAAGCTGATCACTGGCTAAGCTAATAGGGTTTCCGGGATTGGTTTA
GCCTGATACTAATCCAGGTCTACCTTCAGGAGCCAGACCAAACTGCCTAT
TGGCATTGCATTCTTGCAGTAGGGAGGGGAGGTATGGATGGTGTGGAGTC
CACCACAAGGTCCATGCCAGTCTTTGCTGAACCAGCATCAGACTCCATCA
AGCAACAGATGAGAGGTTCCATGATAAAGTGGCCCTCAGCAATCCCCATC
CATTGCTGTCTAGGAAGAACAGTGCTTGTACACAGGTTTAGGACCTCAGT
CTTGGCTGTAATCTTCTGGTTTACTTTGCCAGCACCAAACAGAAGGAAAG
AAAGGGCTCAAATTTGACCAAATAAATTATGCTTCTCCTTCCAGAGATAA
CCTTGAGTCCTGTCTAGGAAGATATTAGAATTGTAAAGAAAAAAAAAATT
ACTCCTTATCCTATGGCAAGTGGAGTCTATGTCTACTTCAGCTGAAATTA
AATCCTGTCCATAATAGATGACCCTTGCTCAAGCTGGCCAGAAGCCATAC
CAACCAGCACGAAGGTTAAAACTATTATTAGTTTTTTCTGTGATTTTCAT
TTTCAGGCCAAGTTTTAGAACAATAAGATTTTAAGAATAGGAAGTAAGTA
AGATTTCTGCATATCCTGTTCTCTTAGTCAGCTGAATTTTTTTTTTTTTT
TTTTTAGTCCTAACTCAGCCTCCCAAAGTGCTGGGATTACAGGCGTGAGC
CACCGCACCAAGCCTGGAATCTATGTCTTACAGTTATGAGAATCAACAGC
TAGCTCATTATGGGCAAGGTGATGTCACTCTGGCTTCTCAATGAAAATGG
CATTTCTCCCTTGGAAAAGGTCATAGCCAGTCAGTCAGTCAGTCACGGGA
GCGCAGCGGCTTCTAGGGGTGAGTGGGACCCACGCGGCCCCACCTGCTCC
TCCCGCGCGCGGCCCCACCCCCCTGCCCCGCCCCGCCTGGTTTATAG

In certain aspects, a promoter is an endogenous human GJB6 promoter as set forth in SEQ ID NO: 101. In some aspects, an promoter sequence is at least 85%, at least 90%, at least 95%, at least 96%, at least 97%, at least 98% at least 99%, or 100% identical to promoter sequence represented by SEQ ID NO: 101. In some aspects, a promoter is an endogenous human GJB6 promoter sequence comprised within SEQ ID NO: 101.

Exemplary Human GJB6 promoter
(SEQ ID NO: 101)
AAATAGCTTCCAACGTTTCCACCCCACCAGCCCTTGCACCACTCCCTGTA
CTGGCCCTGAGCTTTCTAGTCTTGACTGAAAAGCGGGGAGGCAATGTGGT
CTCTCCTGGTGCACTGTCCCGAGGAAGGCCTGCTCCGCTTCCCCGGAGGA
GTCTTCAAAGGATGGAGGTAATTAATAAAAACAACCCCTGTACCTCCTCT
AAGTGGTCATTAATTAATAAAGAACCTCCAGGCTCCTATAGGAGAGGTCT
GTGCACCCCGCGGGCTATGAGAAGGCTGGATCACCCAGAAAGACTGAGGA
TGTGTCCTGGCAAAAACACAGCCTGCCCCTCACACTGCTCCCCACGGGTG
CACTAGGGAGGAAGAGTTCCCTCGAGGGCCTGAGCAGGCGCCCCACACCT
GCACCCGTGCAGAGGGGGCTGGGCCCGCCCTCTGCGCTCCCGAGGGAGAG
CCCTACCCCCTGCATCCCCGGTACCCCGTTCCCTCCAAGGGCCGGAAAGA
GGGCCCCGCGCACTGTGCACTTCTTAGGGGTCCCCCACCCTGCGCCCCCG
CCACGGGAAAAAGGTCCCCGCTCTGCGCATCCGGCCCCGGAGGGACAGCC
CCGGTCCTGCACTCCTTGCTCCTCAGGGGGACGGTCCGCGCCCAGCGGCT
AGTGCGCCCCGGGTAGGTGGGGGCGGGGGGCTCGTCGAGTGACAGCGCTC
GCCTCCCGCAGCCCGCCCGAGCCGCGTCAGGGCAG

In certain aspects, a promoter is an endogenous human PARM1 promoter as set forth in SEQ ID NO: 104. In some aspects, a promoter sequence is at least 85%, at least 90%, at least 95%, at least 96%, at least 97%, at least 98% at least 99%, or 100% identical to promoter sequence represented by SEQ ID NO: 104. In some aspects, a promoter is an endogenous human PARM1 promoter sequence comprised within SEQ ID NO: 104.

Exemplary Human PARM1 promoter
(SEQ ID NO: 104)
TGTACAGGAGATAGTCAGGGAATTAGTAATTTTCAAAGAGGTGACTTTG
AATTCAAACTTAAATATCATCTTCAGCTGAAACAAAGAAGGGGTGCAGT
TATGAGGAAGTGACCAGGTAAAGCATGGCAAACAAAGGTAAAGTTTGTT
ATGCGTATTTAAGTCAGAGCCCTCTCCATTGATAAGAGTTTCCAGTAAT
TTAGTGCCATCCTTTTCTTGCTATAGAGTTCTCGTCTCTATCTGAGCAC
GCAAAAATAACATGCTTTCTTGCTTTCTTGAAGTTGGGCATGGCCATTG
ACTTGCCTTAGCCCATATTTTTCTGTGAAGTGGTCTTCAAAAACCTATA
TTTCTGCCATAGAGTCACTTACTTAACCTGCCCTATTTAAAGGGGCTAA
TGCCTGATAGAATGTCGCTGCATAACTCCATCTGTGTGTGGTCCCTGCA
TCCATGACAACCAAAACCCAGATGCAGAAATTGTTCCTAATCACATAGA
TTACCCTAGAAACCGGAAGGGCCTTGAAGTCAAAAGCATTCAGAGAACA
TGCTGAACAAATTGAATTTGCAGTTTATCTGGCCAGGGAGGATGGAGAG
GGGATGGGCACTTGGTCTGAGTATTTTTTGTTTCTCATTCCAACAGAAA
TTACTAGATTTACCAAAAAATCTACAAGTGGTAGTGTGATAGAGTCAGG
CAGAGGAATTGACCATAGATAAGGTGCTCAGGACTCCTAGAGTCAGCTT
CTGGTATGTGAGAAAGAAGTGAGAACAGAGCCCATGGCATATGAAGAAG
ATATTACAGAAAAAAGAAAGCTGCCTTCCACGCAAATCATTTCTTTACA
AAGGCTTGTTAACTCCTGCAGTGCCAAGAAGCTGAATGCAGCGGCAGAC
ATCCTGGTTCGGGCCCCAGGAAGCTCAGCCGGGTTTAATGTGGATGAGG
GTTTAATGATGTACACGCAGAAGTGTTTTGACAAATGAAGAAGGTCCTC
ATTCTTGGAACATGTGCCGGTTCTCCGAGGGAACTCCTAAAAGGCTGTA
AGCTCATGTAGGAAAAGCTGAGCTAGATTCCTAAGGGCAGAGATGTGCT
CACATTTCTTTGCATCCCTAGTTCCCAGCACAGTGCAAGGCGCTGCAAA
CATTTGCTGAACCCAGGGTCTCGTGTCTTGACTGTCCAGCAGAGGCCGC
TCTGGGCCGGGGCTCTCGGGACCTGAGGGCTGAGAGAAGGAAGGCCAGG
GGGTGGCCCAGTCATCGCCGCGGGGCCCGGGTGGGAGGGGTTTGGCAGC
GGCAGGCGCGGCGGCGGCGGCGGAGGCGGAGGCGGCCCCGGG

Enhancers

In some instances, a construct can include an enhancer sequence. The term “enhancer” refers to a nucleotide sequence that can increase the level of transcription of a nucleic acid encoding a protein of interest (e.g., a connexin 26 protein). Enhancer sequences (generally 50-1500 bp in length) generally increase the level of transcription by providing additional binding sites for transcription-associated proteins (e.g., transcription factors). In some embodiments, an enhancer sequence is found within an intronic sequence. Unlike promoter sequences, enhancer sequences can act at much larger distance away from the transcription start site (e.g., as compared to a promoter). Non-limiting examples of enhancers include a RSV enhancer, a CMV enhancer, and/or a SV40 enhancer. In some embodiments, a construct comprises a CMV enhancer exemplified by SEQ ID NO: 18. In some embodiments, a construct comprises a CMV enhancer exemplified by SEQ ID NO: 63. In some embodiments, a construct comprises a chimeric intron enhancer exemplified by SEQ ID NO: 64. In some embodiments, a construct comprises a GJB2 enhancer exemplified by SEQ ID NO: 65. In some embodiments, an enhancer sequence is at least 85%, at least 90%, at least 95%, at least 96%, at least 97%, at least 98% at least 99%, or 100% identical to the enhancer sequence represented by SEQ ID NO: 18. In some embodiments, an enhancer sequence is at least 85%, at least 90%, at least 95%, at least 96%, at least 97%, at least 98% at least 99%, or 100% identical to the enhancer sequence represented by SEQ ID NO: 63. In some embodiments, an enhancer sequence is at least 85%, at least 90%, at least 95%, at least 96%, at least 97%, at least 98% at least 99%, or 100% identical to the enhancer sequence represented by SEQ ID NO: 64. In some embodiments, an enhancer sequence is at least 85%, at least 90%, at least 95%, at least 96%, at least 97%, at least 98% at least 99%, or 100% identical to the enhancer sequence represented by SEQ ID NO: 65. In some embodiments, an SV-40 derived enhancer is the SV-40 T intron sequence, which is exemplified by SEQ ID NO: 19. In some embodiments, an enhancer sequence is at least 85%, at least 90%, at least 95%, at least 96%, at least 97%, at least 98% at least 99%, or 100% identical to the enhancer sequence represented by SEQ ID NO: 19.

Exemplary CMV enhancer
(SEQ ID NO: 18)
GACATTGATTATTGACTAGTTATTAATAGTAATCAATTACGGGGTCATTA
GTTCATAGCCCATATATGGAGTTCCGCGTTACATAACTTACGGTAAATGG
CCCGCCTGGCTGACCGCCCAACGACCCCCGCCCATTGACGTCAATAATGA
CGTATGTTCCCATAGTAACGCCAATAGGGACTTTCCATTGACGTCAATGG
GTGGACTATTTACGGTAAACTGCCCACTTGGCAGTACATCAAGTGTATCA
TATGCCAAGTACGCCCCCTATTGACGTCAATGACGGTAAATGGCCCGCCT
GGCATTATGCCCAGTACATGACCTTATGGGACTTTCCTACTTGGCAGTAC
ATCTACGTATTAGTCATCGCTATTACCATGG
Exemplary CMV enhancer
(SEQ ID NO: 63)
GACATTGATTATTGACTAGTTATTAATAGTAATCAATTACGGGGTCATTA
GTTCATAGCCCATATATGGAGTTCCGCGTTACATAACTTACGGTAAATGG
CCCGCCTGGCTGACCGCCCAACGACCCCCGCCCATTGACGTCAATAATGA
CGTATGTTCCCATAGTAACGCCAATAGGGACTTTCCATTGACGTCAATGG
GTGGACTATTTACGGTAAACTGCCCACTTGGCAGTACATCAAGTGTATCA
TATGCCAAGTACGCCCCCTATTGACGTCAATGACGGTAAATGGCCCGCCT
GGCATTATGCCCAGTACATGACCTTATGGGACTTTCCTACTTGGCAGTAC
ATCTACGTATTAGTCATCGCTATTACCATGGT
Exemplary SV-40 synthetic intron
(SEQ ID NO: 19)
GGAGTCGCTGCGTTGCCTTCGCCCCGTGCCCCGCTCCGCGCCGCCTCGCG
CCGCCCGCCCCGGCTCTGACTGACCGCGTTACTCCCACAGGTGAGCGGGC
GGGACGGCCCTTCTCCTCCGGGCTGTAATTAGCGCTTGGTTTAATGACGG
CTCGTTTCTTTTCTGTGGCTGCGTGAAAGCCTTAAAGGGCTCCGGGAGGG
CCCTTTGTGCGGGGGGGAGCGGCTCGGGGGGTGCGTGCGTGTGTGTGTGC
GTGGGGAGCGCCGCGTGCGGCCCGCGCTGCCCGGCGGCTGTGAGCGCTGC
GGGCGCGGCGCGGGGCTTTGTGCGCTCCGCGTGTGCGCGAGGGGAGCGCG
GCCGGGGGCGGTGCCCCGCGGTGCGGGGGGGCTGCGAGGGGAACAAAGGC
TGCGTGCGGGGTGTGTGCGTGGGGGGGTGAGCAGGGGGTGTGGGCGCGGC
GGTCGGGCTGTAACCCCCCCCTGCACCCCCCTCCCCGAGTTGCTGAGCAC
GGCCCGGCTTCGGGTGCGGGGCTCCGTGCGGGGCGTGGCGCGGGGCTCGC
CGTGCCGGGCGGGGGGTGGCGGCAGGTGGGGGTGCCGGGCGGGGCGGGGC
CGCCTCGGGCCGGGGAGGGCTCGGGGGAGGGGCGCGGCGGCCCCCGGAGC
GCCGGCGGCTGTCGAGGCGCGGCGAGCCGCAGCCATTGCCTTTTATGGTA
ATCGTGCGAGAGGGCGCAGGGACTTCCTTTGTCCCAAATCTGTGCGGAGC
CGAAATCTGGGAGGCGCCGCCGCACCCCCTCTAGCGGGCGCGGGGCGAAG
CGGTGCGGCGCCGGCAGGAAGGAAATGGGCGGGGAGGGCCTTCGTGCGTC
GCCGCGCCGCCGTCCCCTTCTCCCTCTCCAGCCTCGGGGCTGTCCGCGGG
GGGACGGCTGCCTTCGGGGGGGACGGGGCAGGGCGGGGTTCGGCTTCTGG
CGTGTGACCGGCGGCTCTAGAGCCTCTGCTAACCATGTTCATGCCTTCTT
CTTTTTCCTACAG
Exemplary chimeric intron
(SEQ ID NO: 64)
GGAGTCGCTGCGTTGCCTTCGCCCCGTGCCCCGCTCCGCGCCGCCTCGCG
CCGCCCGCCCCGGCTCTGACTGACCGCGTTACTCCCACAGGTGAGCGGGC
GGGACGGCCCTTCTCCTCCGGGCTGTAATTAGCGCTTGGTTTAATGACGG
CTTGTTTCTTTTCTGTGGCTGCGTGAAAGCCTTGAGGGGCTCCGGGAGCT
AGAGCCTCTGCTAACCATGTTCATGCCTTCTTCTTTTTCCTACAG
Exemplary GJB2 enhancer
(SEQ ID NO: 65)
CTTCTTCTGGAGTCTTTTCTGGAATAATTCTGGGAGTGGGCTCAGCCTGC
GGGAGAGTAACATTTTTATAACTTGATAGATGTAGCTGAGATGCCTCCCA
GAGGGGAGACCCGCCTCTCCTCCGGCAGCTGTGCACGTAGGCTTGTTCCC
AGCAGCCTGGCCAGGGTGGTCCACCTGGTGTTTCTCATCTTCTTTCCCCG
GAGCGCTGACTCCTGCGCGTCCTCTTGGAAGACTCTTGACAGGACGGGTG
TTTTATGGGTGTGATTCAGTGTCCTCTTGCATCAGTTCAATGTGGTGGTG
TTCAATCAACCCTTGTAGCGTTAGCAAAATTTGCTCAAGTCATTCCGCAG
GAATGTCTGTGTCTTGCTTCCAAGAAAGCTTGTAAGTGCCGGCAACAGGC
CAAGCAGCTCACAAACCTGACCACAAGCCTGTGAGTAATTGTGGGGCAGC
ACTTAGCAGTCTTTTATTTTCGACTTATTAAAGTCTCATCTTGGCCTCAC
CTTCTCCCTGGAAGGTGGCGTGGGTGGGAACCACTGGGTCAGATCTTTTT
CACCCTTGCCGTGGAGCCAGTTTCCTGTTGCATGTGGGGGAAGCAACATG
TGGTGAAGAGTATAGAAAACGAAAACATGTGGGTACAGTATGTATAAGTG
GAGGGAACAAACTCATAATTCCAACTAGTTTCTCATGAGAGACTCATGAA
TCATTGTGGTAGTTCTCAATATAAACTTAATCTAGGCCGGATGTGGTGGC
TCACACCTGTAATCTCAGCACTCTGGGTGGATCACTTGAGGTCAGGAGTT
TGAGACCAGTCTGACCAACATGGAGAAACCCCATCGCTACTAAAAATACA
AAATTATCCAGATGTGGTGGCTCACACCTGTAATCCCAGCACTTTGGGAG
GCTGAGGCGGGTGGATCACTTGAGGTCAGGAGTTTGAGACCAGCCTGACC
AACATGGAGAAACTGTGTCTCTACTAAAAATACAAAATTAGCTGGGCGTG
GTGACGCATGCCTGTAATCCCAGCTATTTGGAGGCCGAAGCAGG

Flanking Untranslated Regions, 5′ UTRs and 3′ UTRs

In some embodiments, any of the constructs described herein can include an untranslated region (UTR), such as a 5′ UTR or a 3′ UTR. UTRs of a gene are transcribed but not translated. A 5′ UTR starts at the transcription start site and continues to the start codon but does not include the start codon. A 3′ UTR starts immediately following the stop codon and continues until the transcriptional termination signal. The regulatory and/or control features of a UTR can be incorporated into any of the constructs, compositions, kits, or methods as described herein to enhance or otherwise modulate the expression of a connexin 26 protein.

Natural 5′ UTRs include a sequence that plays a role in translation initiation. in some embodiments, a 5′ UTR can comprise sequences, like Kozak sequences, which are commonly known to be involved in the process by which the ribosome initiates translation of many genes. Kozak sequences have the consensus sequence CCR(A/G)CCAUGG, where R is a purine (A or G) three bases upstream of the start codon (AUG), and the start codon is followed by another “G”. The 5′ UTRs have also been known to form secondary structures that are involved in elongation factor binding.

In some embodiments, a 5′ UTR is included in any of the constructs described herein. Non-limiting examples of 5′ UTRs, including those from the following genes: albumin, serum amyloid A, Apolipoprotein A/B/E, transferrin, alpha fetoprotein, erythropoietin, and Factor VIII, can be used to enhance expression of a nucleic acid molecule, such as an mRNA.

In some embodiments, a 5′ UTR from an mRNA that is transcribed by a cell in the cochlea can be included in any of the constructs, compositions, kits, and methods described herein. In some embodiments, a 5′ UTR is derived from the endogenous GJB2 gene loci and may include all or part of the endogenous sequence exemplified by SEQ ID NO: 20, SEQ ID NO: 21, or SEQ ID NO: 66. In some embodiments, a 5′ UTR sequence is at least 85%, at least 90%, at least 95%, at least 96%, at least 97%, at least 98% at least 99%, or 100% identical to the 5′ UTR sequence represented by SEQ ID NO: 20, SEQ ID NO: 21, or SEQ ID NO: 66.

3′ UTRs are found immediately 3′ to the stop codon of the gene of interest. In some embodiments, a 3′ UTR from an mRNA that is transcribed by a cell in the cochlea can be included in any of the constructs, compositions, kits, and methods described herein. In some embodiments, a 3′ UTR is derived from the endogenous GJB2 gene loci and may include all or part of the endogenous sequence exemplified by SEQ ID NO: 22. In some embodiments, a 3′ UTR sequence is at least 85%, at least 90%, at least 95%, at least 96%, at least 97%, at least 98% at least 99%, or 100% identical to the 3′ UTR sequence represented by SEQ ID NO: 22. In some embodiments, a 3′ UTR is derived from the endogenous GJB2 gene loci and may include all or part of the endogenous sequence exemplified by SEQ ID NO: 67, or SEQ ID NO: 68. In some embodiments, a 3′ UTR sequence is at least 85%, at least 90%, at least 95%, at least 96%, at least 97%, at least 98% at least 99%, or 100% identical to the 3′ UTR sequence represented by SEQ ID NO: 67, or SEQ ID NO: 68.

In some embodiments, a UTR may comprise a non-endogenous regulatory region. In some embodiments, a UTR that comprises a non-endogenous regulatory region is a 3′ UTR. In some embodiments, a UTR that comprises a non-endogenous regulatory region is a 5′ UTR. In some embodiments, a non-endogenous regulatory region may be a target of at least one inhibitory nucleic acid. In some embodiments, an inhibitory nucleic acid inhibits expression and/or activity of a target gene. In some embodiments, an inhibitory nucleic acid is a short interfering RNA (siRNA), a short hairpin RNA (shRNA), a microRNA (miRNA), an antisense oligonucleotide, a guide RNA (gRNA), or a ribozyme. In some embodiments, an inhibitory nucleic acid is an endogenous molecule. In some embodiments, an inhibitory nucleic acid is a non-endogenous molecule. In some embodiments, an inhibitory nucleic acid displays a tissue specific expression pattern. In some embodiments, an inhibitory nucleic acid displays a cell specific expression pattern. In some embodiments, an inhibitory nucleic acid is expressed in inner ear hair cells (e.g., IHCs and/or OHCs). In some aspects, an inhibitory nucleic acid is expressed in inner ear hair cells, spiral ganglion cells, lateral supporting cells, basilar membrane cells, medial supporting cells, spiral limbus cells, inner sulcus cells, or any combination thereof. In some aspects, the inhibitory nucleic acid reduces, suppresses, inhibits, or eliminates expression of Connexin 26. In some aspects, the inhibitory nucleic acid reduces, suppresses, inhibits, or eliminates expression of Connexin 26 in inner ear hair cells, spiral ganglion cells, lateral supporting cells, basilar membrane cells, medial supporting cells, spiral limbus cells, inner sulcus cells, or any combination thereof.

In some aspects, the inhibitory nucleic acid reduces, suppresses, inhibits, or eliminates toxicity associated with the expression of Connexin 26. In some aspects, the inhibitory nucleic acid reduces, suppresses, inhibits, or eliminates toxicity associated with the expression of Connexin 26 in inner ear hair cells, spiral ganglion cells, lateral supporting cells, basilar membrane cells, medial supporting cells, spiral limbus cells, inner sulcus cells, or any combination thereof.

In some embodiments, a construct may comprise more than one non-endogenous regulatory regions, e.g., two, three, four, five, six, seven, eight, nine, or ten regulatory regions. In some embodiments, a construct may comprise four non-endogenous regulatory regions. In some embodiments, a construct may comprise more than one non-endogenous regulatory regions, wherein at least one of the more than one non-endogenous regulatory regions are not the same as at least one of the other non-endogenous regulatory regions.

In some aspects, the disclosure is directed to constructs comprising microRNA regulatory target site (miRTS) which can be used to regulate (e.g., reduce) expression of connexin 26 in a cell (e.g., an inner ear cell, e.g., a hair cell). In some aspects, the constructs provide reduced toxicity that may be associated with expression of connexin 26 in some cells (e.g., an inner ear cell, e.g., a hair cell).

In some embodiments, a non-endogenous regulatory region included in a UTR may comprise a miRNA regulatory target sites (miRTS). In some embodiments, a miRTS may be a human miRNA-182 target sequence. In some embodiments, a UTR may include all or part of the miRNA-182 target sequence. In some embodiments, a UTR may contain more than one miRNA-182 target sequence. In some embodiments, more than one miRNA-182 target sequences may be dispersed at multiple locations in a UTR. In some aspects, the 3′ UTR may include all or part of the miRNA-182 target sequence. In some aspects, the 3′ UTR may contain more than one miRNA-182 target sequence. In some aspects, more than one miRNA-182 target sequences may be dispersed at multiple locations in the 3′ UTR. In some aspects, the miRNA-182 target sequence comprises the nucleic acid sequence with at least 85%, at least 90%, at least 95%, at least 96%, at least 97%, at least 98%, at least 99%, or 100% identity to SEQ ID NO: 78. In some aspects, the miRNA-182 target sequence comprises the nucleic acid sequence of SEQ ID NO: 78.

In some embodiments, a miRTS may be a human miRNA-183 target sequence. In some embodiments, a UTR may include all or part of the miRNA-183 target sequence. In some embodiments, a UTR may contain more than one miRNA-183 target sequence. In some embodiments, more than one miRNA-183 target sequences may be dispersed at multiple locations in a UTR. In some aspects, the 3′ UTR may include all or part of the miRNA-183 target sequence. In some aspects, the 3′ UTR may contain more than one miRNA-183 target sequence. In some aspects, more than one miRNA-183 target sequences may be dispersed at multiple locations in the 3′ UTR. In some aspects, the miRNA-183 target sequence comprises the nucleic acid sequence with at least 85%, at least 90%, at least 95%, at least 96%, at least 97%, at least 98%, at least 99%, or 100% identity to SEQ ID NO: 79. In some aspects, the miRNA-183 target sequence comprises the nucleic acid sequence of SEQ ID NO: 79.

In some aspects, a miRTS may be a human miRNA-194 target sequence. In some aspects, a UTR may include all or part of the miRNA-194 target sequence. In some aspects, a UTR may contain more than one miRNA-194 target sequence. In some aspects, more than one miRNA-194 target sequences may be dispersed at multiple locations in a UTR. In some aspects, the 3′ UTR may include all or part of the miRNA-194 target sequence. In some aspects, the 3′ UTR may contain more than one miRNA-194 target sequence. In some aspects, more than one miRNA-194 target sequences may be dispersed at multiple locations in the 3′ UTR. In some aspects, the miRNA-194 target sequence comprises the nucleic acid sequence with at least 85%, at least 90%, at least 95%, at least 96%, at least 97%, at least 98%, at least 99%, or 100% identity to SEQ ID NO: 107. In some aspects, the miRNA-194 target sequence comprises the nucleic acid sequence of SEQ ID NO: 107.

In some aspects, a miRTS may be a human miRNA-140 target sequence. In some aspects, a UTR may include all or part of the miRNA-140 target sequence. In some aspects, a UTR may contain more than one miRNA-140 target sequence. In some aspects, more than one miRNA-140 target sequences may be dispersed at multiple locations in a UTR. In some aspects, the 3′ UTR may include all or part of the miRNA-140 target sequence. In some aspects, the 3′ UTR may contain more than one miRNA-140 target sequence. In some aspects, more than one miRNA-140 target sequences may be dispersed at multiple locations in the 3′ UTR. In some aspects, the miRNA-140 target sequence comprises the nucleic acid sequence with at least 85%, at least 90%, at least 95%, at least 96%, at least 97%, at least 98%, at least 99%, or 100% identity to SEQ ID NO: 108. In some aspects, the miRNA-140 target sequence comprises the nucleic acid sequence of SEQ ID NO: 108.

In some aspects, a miRTS may be a human miRNA-18a target sequence. In some aspects, a UTR may include all or part of the miRNA-18a target sequence. In some aspects, a UTR may contain more than one miRNA-18a target sequence. In some aspects, more than one miRNA-18a target sequences may be dispersed at multiple locations in a UTR. In some aspects, the 3′ UTR may include all or part of the miRNA-18a target sequence. In some aspects, the 3′ UTR may contain more than one miRNA-18a target sequence. In some aspects, more than one miRNA-18a target sequences may be dispersed at multiple locations in the 3′ UTR. In some aspects, the miRNA-18a target sequence comprises the nucleic acid sequence with at least 85%, at least 90%, at least 95%, at least 96%, at least 97%, at least 98%, at least 99%, or 100% identity to SEQ ID NO: 109. In some aspects, the miRNA-18a target sequence comprises the nucleic acid sequence of SEQ ID NO: 109.

In some aspects, a miRTS may be a human miRNA-99a target sequence. In some aspects, a UTR may include all or part of the miRNA-99a target sequence. In some aspects, a UTR may contain more than one miRNA-99a target sequence. In some aspects, more than one miRNA-99a target sequences may be dispersed at multiple locations in a UTR. In some aspects, the 3′ UTR may include all or part of the miRNA-99a target sequence. In some aspects, the 3′ UTR may contain more than one miRNA-99a target sequence. In some aspects, more than one miRNA-99a target sequences may be dispersed at multiple locations in the 3′ UTR. In some aspects, the miRNA-99a target sequence comprises the nucleic acid sequence with at least 85%, at least 90%, at least 95%, at least 96%, at least 97%, at least 98%, at least 99%, or 100% identity to SEQ ID NO: 110. In some aspects, the miRNA-99a target sequence comprises the nucleic acid sequence of SEQ ID NO: 110.

In some aspects, a miRTS may be a human miRNA-30b target sequence. In some aspects, a UTR may include all or part of the miRNA-30b target sequence. In some aspects, a UTR may contain more than one miRNA-30b target sequence. In some aspects, more than one miRNA-30b target sequences may be dispersed at multiple locations in a UTR. In some aspects, the 3′ UTR may include all or part of the miRNA-30b target sequence. In some aspects, the 3′ UTR may contain more than one miRNA-30b target sequence. In some aspects, more than one miRNA-30b target sequences may be dispersed at multiple locations in the 3′ UTR. In some aspects, the miRNA-30b target sequence comprises the nucleic acid sequence with at least 85%, at least 90%, at least 95%, at least 96%, at least 97%, at least 98%, at least 99%, or 100% identity to SEQ ID NO: 111. In some aspects, the miRNA-30b target sequence comprises the nucleic acid sequence of SEQ ID NO: 111.

In some aspects, a miRTS may be a human miRNA-15a target sequence. In some aspects, a UTR may include all or part of the miRNA-15a target sequence. In some aspects, a UTR may contain more than one miRNA-15a target sequence. In some aspects, more than one miRNA-15a target sequences may be dispersed at multiple locations in a UTR. In some aspects, the 3′ UTR may include all or part of the miRNA-15a target sequence. In some aspects, the 3′ UTR may contain more than one miRNA-15a target sequence. In some aspects, more than one miRNA-15a target sequences may be dispersed at multiple locations in the 3′ UTR. In some aspects, the miRNA-15a target sequence comprises the nucleic acid sequence with at least 85%, at least 90%, at least 95%, at least 96%, at least 97%, at least 98%, at least 99%, or 100% identity to SEQ ID NO: 112. In some aspects, the miRNA-15a target sequence comprises the nucleic acid sequence of SEQ ID NO: 112.

In some aspects, the miRTS may be a target sequence for a miRNA that is expressed in specific cells of the inner ear. In some aspects, the miRTS may be a target sequence for a miRNA that is expressed in ear hair cells, spiral ganglion cells, lateral supporting cells, basilar membrane cells, medial supporting cells, spiral limbus cells, inner sulcus cells, or any combination thereof.

In some aspects, the miRTS may be a target sequence for a miRNA that is expressed in ear hair cells. In some aspects, the miRNA that is expressed in ear hair cells reduces, decreases, or suppresses expression of the GJB2 protein (Connexin 26). In some aspects, miRNAs that are expressed in ear hair cells are miR-194, miR-140, miR-18a, miR-99a, miR-30b, miR-15a, miR182, or miR-183. In some aspects, the miRNA that is expressed in ear hair cells is miR-194. In some aspects, the miRNA-194 target sequence comprises the nucleic acid sequence with at least 85%, at least 90%, at least 95%, at least 96%, at least 97%, at least 98%, at least 99%, or 100% identity to SEQ ID NO: 107. In some aspects, the miRNA-194 target sequence comprises the nucleic acid sequence of SEQ ID NO: 107. In some aspects, the miRNA that is expressed in ear hair cells is miR-140. In some aspects, the miRNA-140 target sequence comprises the nucleic acid sequence with at least 85%, at least 90%, at least 95%, at least 96%, at least 97%, at least 98%, at least 99%, or 100% identity to SEQ ID NO: 108. In some aspects, the miRNA-140 target sequence comprises the nucleic acid sequence of SEQ ID NO: 108. In some aspects, the miRNA that is expressed in ear hair cells is miR-18a. In some aspects, the miRNA-18a target sequence comprises the nucleic acid sequence with at least 85%, at least 90%, at least 95%, at least 96%, at least 97%, at least 98%, at least 99%, or 100% identity to SEQ ID NO: 109. In some aspects, the miRNA-18a target sequence comprises the nucleic acid sequence of SEQ ID NO: 109. In some aspects, the miRNA that is expressed in ear hair cells is miR-99a. In some aspects, the miRNA-99a target sequence comprises the nucleic acid sequence with at least 85%, at least 90%, at least 95%, at least 96%, at least 97%, at least 98%, at least 99%, or 100% identity to SEQ ID NO: 110. In some aspects, the miRNA-99a target sequence comprises the nucleic acid sequence of SEQ ID NO: 110. In some aspects, the miRNA that is expressed in ear hair cells is miR-30b. In some aspects, the miRNA-30b target sequence comprises the nucleic acid sequence with at least 85%, at least 90%, at least 95%, at least 96%, at least 97%, at least 98%, at least 99%, or 100% identity to SEQ ID NO: 111. In some aspects, the miRNA-30b target sequence comprises the nucleic acid sequence of SEQ ID NO: 111. In some aspects, the miRNA that is expressed in ear hair cells is miR-15a. In some aspects, the miRNA-15a target sequence comprises the nucleic acid sequence with at least 85%, at least 90%, at least 95%, at least 96%, at least 97%, at least 98%, at least 99%, or 100% identity to SEQ ID NO: 112. In some aspects, the miRNA-15a target sequence comprises the nucleic acid sequence of SEQ ID NO: 112. In some aspects, the miRNA that is expressed in ear hair cells is miR-182. In some aspects, the miRNA-182 target sequence comprises the nucleic acid sequence with at least 85%, at least 90%, at least 95%, at least 96%, at least 97%, at least 98%, at least 99%, or 100% identity to SEQ ID NO: 78. In some aspects, the miRNA-182 target sequence comprises the nucleic acid sequence of SEQ ID NO: 78. In some aspects, the miRNA that is expressed in ear hair cells is miR-183. In some aspects, the miRNA-183 target sequence comprises the nucleic acid sequence with at least 85%, at least 90%, at least 95%, at least 96%, at least 97%, at least 98%, at least 99%, or 100% identity to SEQ ID NO: 79. In some aspects, the miRNA-183 target sequence comprises the nucleic acid sequence of SEQ ID NO: 79.

In some aspects, the miRTS may be a target sequence for a miRNA that is expressed in the spiral ganglion cells. In some aspects, the miRNA that is expressed in the spiral ganglion cells reduces, decreases, or suppresses expression of the GJB2 protein (Connexin 26). In some aspects, miRNAs that are expressed in the spiral ganglion cells are miR-194, miR-18a, miR-99a, miR-30b, miR-15a, miR182, or miR-183. In some aspects, the miRNA that is expressed in ear hair cells is miR-194. In some aspects, the miRNA-194 target sequence comprises the nucleic acid sequence with at least 85%, at least 90%, at least 95%, at least 96%, at least 97%, at least 98%, at least 99%, or 100% identity to SEQ ID NO: 107. In some aspects, the miRNA-194 target sequence comprises the nucleic acid sequence of SEQ ID NO: 107. In some aspects, the miRNA that is expressed in ear hair cells is miR-18a. In some aspects, the miRNA-18a target sequence comprises the nucleic acid sequence with at least 85%, at least 90%, at least 95%, at least 96%, at least 97%, at least 98%, at least 99%, or 100% identity to SEQ ID NO: 109. In some aspects, the miRNA-18a target sequence comprises the nucleic acid sequence of SEQ ID NO: 109. In some aspects, the miRNA that is expressed in ear hair cells is miR-99a. In some aspects, the miRNA-99a target sequence comprises the nucleic acid sequence with at least 85%, at least 90%, at least 95%, at least 96%, at least 97%, at least 98%, at least 99%, or 100% identity to SEQ ID NO: 110. In some aspects, the miRNA-99a target sequence comprises the nucleic acid sequence of SEQ ID NO: 110. In some aspects, the miRNA that is expressed in ear hair cells is miR-30b. In some aspects, the miRNA-30b target sequence comprises the nucleic acid sequence with at least 85%, at least 90%, at least 95%, at least 96%, at least 97%, at least 98%, at least 99%, or 100% identity to SEQ ID NO: 111. In some aspects, the miRNA-30b target sequence comprises the nucleic acid sequence of SEQ ID NO: 111. In some aspects, the miRNA that is expressed in ear hair cells is miR-15a. In some aspects, the miRNA-15a target sequence comprises the nucleic acid sequence with at least 85%, at least 90%, at least 95%, at least 96%, at least 97%, at least 98%, at least 99%, or 100% identity to SEQ ID NO: 112. In some aspects, the miRNA-15a target sequence comprises the nucleic acid sequence of SEQ ID NO: 112. In some aspects, the miRNA that is expressed in ear hair cells is miR-182. In some aspects, the miRNA-182 target sequence comprises the nucleic acid sequence with at least 85%, at least 90%, at least 95%, at least 96%, at least 97%, at least 98%, at least 99%, or 100% identity to SEQ ID NO: 78. In some aspects, the miRNA-182 target sequence comprises the nucleic acid sequence of SEQ ID NO: 78. In some aspects, the miRNA that is expressed in ear hair cells is miR-183. In some aspects, the miRNA-183 target sequence comprises the nucleic acid sequence with at least 85%, at least 90%, at least 95%, at least 96%, at least 97%, at least 98%, at least 99%, or 100% identity to SEQ ID NO: 79. In some aspects, the miRNA-183 target sequence comprises the nucleic acid sequence of SEQ ID NO: 79.

In some aspects, the miRTS may be a target sequence for a miRNA that is expressed in basilar membrane cells. In some aspects, the miRNA that is expressed in the basilar membrane cells reduces, decreases, or suppresses expression of the GJB2 protein (Connexin 26). In some aspects, miRNAs that are expressed in basilar membrane cells are miR-99a, miR-30b, and miR-15a. In some aspects, the miRNA that is expressed in ear hair cells is miR-99a. In some aspects, the miRNA-99a target sequence comprises the nucleic acid sequence with at least 85%, at least 90%, at least 95%, at least 96%, at least 97%, at least 98%, at least 99%, or 100% identity to SEQ ID NO: 110. In some aspects, the miRNA-99a target sequence comprises the nucleic acid sequence of SEQ ID NO: 110. In some aspects, the miRNA that is expressed in ear hair cells is miR-30b. In some aspects, the miRNA-30b target sequence comprises the nucleic acid sequence with at least 85%, at least 90%, at least 95%, at least 96%, at least 97%, at least 98%, at least 99%, or 100% identity to SEQ ID NO: 111. In some aspects, the miRNA-30b target sequence comprises the nucleic acid sequence of SEQ ID NO: 111. In some aspects, the miRNA that is expressed in ear hair cells is miR-15a. In some aspects, the miRNA-15a target sequence comprises the nucleic acid sequence with at least 85%, at least 90%, at least 95%, at least 96%, at least 97%, at least 98%, at least 99%, or 100% identity to SEQ ID NO: 112. In some aspects, the miRNA-15a target sequence comprises the nucleic acid sequence of SEQ ID NO: 112.

In some aspects, the miRTS may be a target sequence for a miRNA that is expressed in lateral supporting cells. In some aspects, the miRNA that is expressed in lateral supporting cells reduces, decreases, or suppresses expression of the GJB2 protein (Connexin 26). In some aspects, miRNAs that are expressed in lateral supporting cells are miR-99a, miR-30b, and miR-15a. In some aspects, the miRNA that is expressed in ear hair cells is miR-99a. In some aspects, the miRNA-99a target sequence comprises the nucleic acid sequence with at least 85%, at least 90%, at least 95%, at least 96%, at least 97%, at least 98%, at least 99%, or 100% identity to SEQ ID NO: 110. In some aspects, the miRNA-99a target sequence comprises the nucleic acid sequence of SEQ ID NO: 110. In some aspects, the miRNA that is expressed in ear hair cells is miR-30b. In some aspects, the miRNA-30b target sequence comprises the nucleic acid sequence with at least 85%, at least 90%, at least 95%, at least 96%, at least 97%, at least 98%, at least 99%, or 100% identity to SEQ ID NO: 111. In some aspects, the miRNA-30b target sequence comprises the nucleic acid sequence of SEQ ID NO: 111. In some aspects, the miRNA that is expressed in ear hair cells is miR-15a. In some aspects, the miRNA-15a target sequence comprises the nucleic acid sequence with at least 85%, at least 90%, at least 95%, at least 96%, at least 97%, at least 98%, at least 99%, or 100% identity to SEQ ID NO: 112. In some aspects, the miRNA-15a target sequence comprises the nucleic acid sequence of SEQ ID NO: 112.

In some aspects, the miRTS may be a target sequence for a miRNA that is expressed in medial supporting cells. In some aspects, the miRNA that is expressed in medial supporting cells reduces, decreases, or suppresses expression of the GJB2 protein (Connexin 26). In some aspects, miRNAs that are expressed in medial supporting cells are miR182 and miR-183. In some aspects, the miRNA that is expressed in ear hair cells is miR-182. In some aspects, the miRNA-182 target sequence comprises the nucleic acid sequence with at least 85%, at least 90%, at least 95%, at least 96%, at least 97%, at least 98%, at least 99%, or 100% identity to SEQ ID NO: 78. In some aspects, the miRNA-182 target sequence comprises the nucleic acid sequence of SEQ ID NO: 78. In some aspects, the miRNA that is expressed in ear hair cells is miR-183. In some aspects, the miRNA-183 target sequence comprises the nucleic acid sequence with at least 85%, at least 90%, at least 95%, at least 96%, at least 97%, at least 98%, at least 99%, or 100% identity to SEQ ID NO: 79. In some aspects, the miRNA-183 target sequence comprises the nucleic acid sequence of SEQ ID NO: 79.

In some aspects, the miRTS may be a target sequence for a miRNA that is expressed in spiral limbus cells. In some aspects, the miRNA that is expressed in spiral limbus cells reduces, decreases, or suppresses expression of the GJB2 protein (Connexin 26). In some aspects, miRNAs that are expressed in spiral limbus cells are miR182 and miR-183. In some aspects, the miRNA that is expressed in ear hair cells is miR-182. In some aspects, the miRNA-182 target sequence comprises the nucleic acid sequence with at least 85%, at least 90%, at least 95%, at least 96%, at least 97%, at least 98%, at least 99%, or 100% identity to SEQ ID NO: 78. In some aspects, the miRNA-182 target sequence comprises the nucleic acid sequence of SEQ ID NO: 78. In some aspects, the miRNA that is expressed in ear hair cells is miR-183. In some aspects, the miRNA-183 target sequence comprises the nucleic acid sequence with at least 85%, at least 90%, at least 95%, at least 96%, at least 97%, at least 98%, at least 99%, or 100% identity to SEQ ID NO: 79. In some aspects, the miRNA-183 target sequence comprises the nucleic acid sequence of SEQ ID NO: 79.

In some aspects, the miRTS may be a target sequence for a miRNA that is expressed in inner sulcus cells. In some aspects, the miRNA that is expressed in inner sulcus cells reduces, decreases, or suppresses expression of the GJB2 protein (Connexin 26). In some aspects, miRNAs that are expressed in inner sulcus cells are miR182 and miR-183. In some aspects, the miRNA that is expressed in ear hair cells is miR-182. In some aspects, the miRNA-182 target sequence comprises the nucleic acid sequence with at least 85%, at least 90%, at least 95%, at least 96%, at least 97%, at least 98%, at least 99%, or 100% identity to SEQ ID NO: 78. In some aspects, the miRNA-182 target sequence comprises the nucleic acid sequence of SEQ ID NO: 78. In some aspects, the miRNA that is expressed in ear hair cells is miR-183. In some aspects, the miRNA-183 target sequence comprises the nucleic acid sequence with at least 85%, at least 90%, at least 95%, at least 96%, at least 97%, at least 98%, at least 99%, or 100% identity to SEQ ID NO: 79. In some aspects, the miRNA-183 target sequence comprises the nucleic acid sequence of SEQ ID NO: 79.

In some embodiments, a non-endogenous regulatory region included in a UTR may comprise multiple miRNA regulatory target sites (miRTS). In some embodiments, a UTR may comprise at least one miRNA-182 target site and at least one miRNA-183 target site. In some embodiments, a non-endogenous regulatory region included in a UTR is a destabilizing domain, and is exemplified by SEQ ID NO: 80. In some embodiments, a UTR may include a sequence that is at least 85%, at least 90%, at least 95%, at least 96%, at least 97%, at least 98% at least 99%, or 100% identical to a non-endogenous regulatory region exemplified by SEQ ID NO: 80.

3′ UTRs are known to have stretches of adenosines and uridines (in the RNA form) or thymidines (in the DNA form) embedded in them. These AU-rich signatures are particularly prevalent in genes with high rates of turnover. Based on their sequence features and functional properties, the AU-rich elements (AREs) can be separated into three classes (Chen et al., Mol. Cell. Biol. 15:5777-5788, 1995; Chen et al., Mol. Cell Biol. 15:2010-2018, 1995, each of which is incorporated herein by reference in its entirety): Class I AREs contain several dispersed copies of an AUUUA motif within U-rich regions. For example, c-Myc and MyoD mRNAs contain class I AREs. Class II AREs possess two or more overlapping UUAUUUA(U/A) (U/A) nonamers. GM-CSF and TNF-alpha mRNAs are examples that contain class II AREs. Class III AREs are less well defined. These U-rich regions do not contain an AUUUA motif, two well-studied examples of this class are c-Jun and myogenin mRNAs.

Most proteins binding to the AREs are known to destabilize the messenger, whereas members of the ELAV family, most notably HuR, have been documented to increase the stability of mRNA. HuR binds to AREs of all the three classes. Engineering the HuR specific binding sites into the 3′ UTR of nucleic acid molecules will lead to HuR binding and thus, stabilization of the message in vivo.

In some embodiments, the introduction, removal, or modification of 3′ UTR AREs can be used to modulate the stability of an mRNA encoding a connexin 26 protein. In other embodiments, AREs can be removed or mutated to increase the intracellular stability and thus increase translation and production of a connexin 26 protein.

In other embodiments, non-ARE sequences may be incorporated into the 5′ or 3′ UTRs. In some embodiments, introns or portions of intron sequences may be incorporated into the flanking regions of the polynucleotides in any of the constructs, compositions, kits, and methods provided herein. Incorporation of intronic sequences may increase protein production as well as mRNA levels.

Exemplary 5′ UTR Sequence
(SEQ ID NO: 20)
GTTGCGGCCCCGCAGCGCCCGCGCGCTCCTCTCCCCGACTCGGAGCCCCTCGGCGGCGCCCGGCC
CAGGACCCGCCTAGGAGCGCAGGAGCCCCAGCGCAGAGACCCCAACGCCGAGACCCCCGCCCCGG
CCCCGCCGCGCTTCCTCCCGACGCAGAGCAAACCGCCCAGAGTAGAAG
Exemplary 5′ UTR Sequence
(SEQ ID NO: 21)
TTTAGGACCCTTGTTCGCGAAGAGGTGGTGTGCGGCTGAGACCCGCGTCCTCAGGACGGTTCCAT
CAGTGCCTCGATCCTGCCCCACTGGAGGAGGAAGGCAGCCCGAACAGCGCTCACCTAACTAACAG
CTGCTGAGAGCTGGGTTCCGTGGCCATGCACCTGGGACTGCCTTGAGAAGCGTGAGCAAACCGCC
CAGAGTAGAAG
Exemplary 5′ UTR Sequence
(SEQ ID NO: 66)
GTTGCGGCCCCGCAGCGCCCGCGCGCTCCTCTCCCCGACTCGGAGCCCCTCGGCGGCGCCCGGCC
CAGGACCCGCCTAGGAGCGCAGGAGCCCCAGCGCAGAGACCCCAACGCCGAGACCCCCGCCCCGG
CCCCGCCGCGCTTCCTCCCGACGCAGTTTAGGACCCTTGTTCGCGAAGAGGTGGTGTGCGGCTGA
GACCCGCGTCCTCAGGACGGTTCCATCAGTGCCTCGATCCTGCCCCACTGGAGGAGGAAGGCAGC
CCGAACAGCGCTCACCTAACTAACAGCTGCTGAGAGCTGGGTTCCGTGGCCATGCACCTGGGACT
GCCTTGAGAAGCGTGAGCAAACCGCCCAGAGTAGAAG
Exemplary 3′ UTR Sequence
(SEQ ID NO: 22)
CGCATTGCCCAGTTGTTAGATTAAGAAATAGACAGCATGAGAGGGATGAGGCAACCCGTGCTCAG
CTGTCAAGGCTCAGTCGCTAGCATTTCCCAACACAAAGATTCTGACCTTAAATGCAACCATTTGA
AACCCCTGTAGGCCTCAGGTGAAACTCCAGATGCCACAATGGAGCCTCTGCTCCCCTAAAGCCTC
AAAACAAAGGCCTAATTCTATGCCTGTCTTAATTTTCTTTCACTTAAGTTAGTTCCACTGAGACC
CCAGGCTGTTAGGGGTTATTGGTGTAAGGTACTTTCATATTTTAAACAGAGGATATCGGCATTTG
TTTCTTTCTCTGAGGACAAGAGAAAAAAGCCAGGTTCCACAGAGGACACAGAGAAGGTTTGGGTG
TCCTCCTGGGGTTCTTTTTGCCAACTTTCCCCACGTTAAAGGTGAACATTGGTTCTTTCATTTGC
TTTGGAAGTTTTAATCTCTAACAGTGGACAAAGTTACCAGTGCCTTAAACTCTGTTACACTTTTT
GGAAGTGAAAACTTTGTAGTATGATAGGTTATTTTGATGTAAAGATGTTCTGGATACCATTATAT
GTTCCCCCTGTTTCAGAGGCTCAGATTGTAATATGTAAATGGTATGTCATTCGCTACTATGATTT
AATTTGAAATATGGTCTTTTGGTTATGAATACTTTGCAGCACAGCTGAGAGGCTGTCTGTTGTAT
TCATTGTGGTCATAGCACCTAACAACATTGTAGCCTCAATCGAGTGAGACAGACTAGAAGTTCCT
AGTGATGGCTTATGATAGCAAATGGCCTCATGTCAAATATTTAGATGTAATTTTGTGTAAGAAAT
ACAGACTGGATGTACCACCAACTACTACCTGTAATGACAGGCCTGTCCAACACATCTCCCTTTTC
CATGACTGTGGTAGCCAGCATCGGAAAGAACGCTGATTTAAAGAGGTCGCTTGGGAATTTTATTG
ACACAGTACCATTTAATGGGGAGGACAAAATGGGGCAGGGGAGGGAGAAGTTTCTGTCGTTAAAA
ACAGATTTGGAAAGACTGGACTCTAAAGTCTGTTGATTAAAGATGAGCTTTGTCTACTTCAAAAG
TTTGTTTGCTTACCCCTTCAGCCTCCAATTTTTTAAGTGAAAATATAGCTAATAACATGTGAAAA
GAATAGAAGCTAAGGTTTAGATAAATATTGAGCAGATCTATAGGAAGATTGAACCTGAATATTGC
CATTATGCTTGACATGGTTTCCAAAAAATGGTACTCCACATATTTCAGTGAGGGTAAGTATTTTC
CTGTTGTCAAGAATAGCATTGTAAAAGCATTTTGTAATAATAAAGAATAGCTTTAATGATATGCT
TGTAACTAAAATAATTTTGTAATGTATCAAATACATTTAAAACATTAAAATATAATCTCTATAAT
AA
Exemplary 3′ UTR Sequence
(SEQ ID NO: 67)
GAAATAGACAGCATGAGAGGGATGAGGCAACCCGTGCTCAGCTGTCAAGGCTCAGTCGCTAGCAT
TTCCCAACACAAAGATTCTGACCTTAAATGCAACCATTTGAAACCCCTGTAGGCCTCAGGTGAAA
CTCCAGATGCCACAATGGAGCTCTGCTCCCCTAAAGCCTCAAAACAAAGGCCTAATTCTATGCCT
GTCTTAATTTTCTTTCACTTAAGTTAGTTCCACTGAGACCCCAGGCTGTTAGGGGTTATTGGTGT
AAGGTACTTTCATATTTTAAACAGAGGATATCGGCATTTGTTTCTTTCTCTGAGGACAAGAGAAA
AAAGCCAGGTTCCACAGAGGACACAGAGAAGGTTTGGGTGTCCTCCTGGGGTTCTTTTTGCCAAC
TTTCCCCACGTTAAAGGTGAACATTGGTTCTTTCATTTGCTTTGGAAGTTTTAATCTCTAACAGT
GGACAAAGTTACCAGTGCCTTAAACTCTGTTACACTTTTTGGAAGTGAAAACTTTGTAGTATGAT
AGGTTATTTTGATGTAAAGATGTTCTGGATACCATTATATGTTCCCCCTGTTTCAGAGGCTCAGA
TTGTAATATGTAAATGGTATGTCATTCGCTACTATGATTTAATTTGAAATATGGTCTTTTGGTTA
TGAATACTTTGCAGCACAGCTGAGAGGCTGTCTGTTGTATTCATTGTGGTCATAGCACCTAACAA
CATTGTAGCCTCAATCGAGTGAGACAGACTAGAAGTTCCTAGTGATGGCTTATGATAGCAAATGG
CCTCATGTCAAATATTTAGATGTAATTTTGTGTAAGAAATACAGACTGGATGTACCACCAACTAC
TACCTGTAATGACAGGCCTGTCCAACACATCTCCCTTTTCCATGACTGTGGTAGCCAGCATCGGA
AAGAACGCTGATTTAAAGAGGTCGCTTGGGAATTTTATTGACACAGTACCATTTAATGGGGAGGA
CAAAATGGGGCAGGGGAGGGAGAAGTTTCTGTCGTTAAAAACAGATTTGGAAAGACTGGACTCTA
AAGTCTGTTGATTAAAGATGAGCTTTGTCTACTTCAAAAGTTTGTTTGCTTACCCCTTCAGCCTC
CAATTTTTTAAGTGAAAATATAGCTAATAACATGTGAAAAGAATAGAAGCTAAGGTTTAGATAAA
TATTGAGCAGATCTATAGGAAGATTGAACCTGAATATTGCCATTATGCTTGACATGGTTTCCAAA
AAATGGTACTCCACATATTTCAGTGAGGGTAAGTATTTTCCTGTTGTCAAGAATAGCATTGTAAA
AGCATTTTGTAATAATAAAGAATAGCTTTAATGATATGCTTGTAACTAAAATAATTTTGTAATGT
ATCAAATACATTTAAAACATTAAAATATAATCTCTATAATAA
Exemplary 3′ UTR Sequence
(SEQ ID NO: 68)
CGCATTGCCCAGTTGTTAGATTAAGAAATAGACAGCATGAGAGGGATGAGGCAACCCGTGCTCAG
CTGTCAAGGCTCAGTCGCTAGCATTTCCCAACACAAAGATTCTGACCTTAAATGCAACCATTTGA
AACCCCTGTAGGCCTCAGGTGAAACTCCAGATGCCACAATGGAGCCTCTGCTCCCCTAAAGCCTC
AAAACAAAGGCCTAATTCTATGCCTGTCTTAATTTTCTTTCACTTAAGTTAGTTCCACTGAGACC
CCAGGCTGTTAGGGGTTATTGGTGTAAGGTACTTTCATATTTTAAACAGAGGATATCGGCATTTG
TTTCTTTCTCTGAGGACAAGAGAAAAAAGCCAGGTTCCACAGAGGACACAGAGAAGGTTTGGGTG
TCCTCCTGGGGTTCTTTTTGCCAACTTTCCCCACGTTAAAGGTGAACATTGGTTCTTTCATTTGC
TTTGGAAGTTTTAATCTCTAACAGTGGACAAAGTTACCAGTGCCTTAAACTCTGTTACACTTTTT
GGAAGTGAAAACTTTGTAGTATGATAGGTTATTTTGATGTAAAGATGTTCTGGATACCATTATAT
GTTCCCCCTGTTTCAGAGGCTCAGATTGTAATATGTAAATGGTATGTCATTCGCTACTATGATTT
AATTTGAAATATGGTCTTTTGGTTATGAATACTTTGCAGCACAGCTGAGAGGCTGTCTGTTGTAT
TCATTGTGGTCATAGCACCTAACAACATTGTAGCCTCAATCGAGTGAGACAGACTAGAAGTTCCT
AGTGATGGCTTATGATAGCAAATGGCCTCATGTCAAATATTTAGATGTAATTTTGTGTAAGAAAT
ACAGACTGGATGTACCACCAACTACTACCTGTAATGACAGGCCTGTCCAACACATCTCCCTTTTC
CATGACTGTGGTAGCCAGCATCGGAAAGAACGCTGATTTAAAGAGGTCGCTTGGGAATTTTATTG
ACACAGTACCATTTAATGGGGAGGACAAAATGGGGCAGGGGAGGGAGAAGTTTCTGTCGTTAAAA
ACAGATTTGGAAAGACTGGACTCTAAAGTCTGTTGATTAAAGATGAGCTTTGTCTACTTCAAAAG
TTTGTTTGCTTACCCCTTCAGCCTCCAATTTTTTAAGTGAAAATATAGCTAATAACATGTGAAAA
GAATAGAAGCTAAGGTTTAGATAAATATTGAGCAGATCTATAGGAAGATTGAACCTGAATATTGC
CATTATGCTTGAC
Exemplary 3′ UTR Sequence
(SEQ ID NO: 69)
GAGCTCAGTGTGAGTTCTACCATTGCCAAACTCGAGCAGTGAATTCTACCAGTGCCATAGGATCC
AGTGTGAGTTCTACCATTGCCAAAGGTACCCAGTGAATTCTACCAGTGCCATAGTTAACCGCATT
GCCCAGTTGTTAGATTAAGAAATAGACAGCATGAGAGGGATGAGGCAACCCGTGCTCAGCTGTCA
AGGCTCAGTCGCTAGCATTTCCCAACACAAAGATTCTGACCTTAAATGCAACCATTTGAAACCCC
TGTAGGCCTCAGGTGAAACTCCAGATGCCACAATGGAGCCTCTGCTCCCCTAAAGCCTCAAAACA
AAGGCCTAATTCTATGCCTGTCTTAATTTTCTTTCACTTAAGTTAGTTCCACTGAGACCCCAGGC
TGTTAGGGGTTATTGGTGTAAGGTACTTTCATATTTTAAACAGAGGATATCGGCATTTGTTTCTT
TCTCTGAGGACAAGAGAAAAAAGCCAGGTTCCACAGAGGACACAGAGAAGGTTTGGGTGTCCTCC
TGGGGTTCTTTTTGCCAACTTTCCCCACGTTAAAGGTGAACATTGGTTCTTTCATTTGCTTTGGA
AGTTTTAATCTCTAACAGTGGACAAAGTTACCAGTGCCTTAAACTCTGTTACACTTTTTGGAAGT
GAAAACTTTGTAGTATGATAGGTTATTTTGATGTAAAGATGTTCTGGATACCATTATATGTTCCC
CCTGTTTCAGAGGCTCAGATTGTAATATGTAAATGGTATGTCATTCGCTACTATGATTTAATTTG
AAATATGGTCTTTTGGTTATGAATACTTTGCAGCACAGCTGAGAGGCTGTCTGTTGTATTCATTG
TGGTCATAGCACCTAACAACATTGTAGCCTCAATCGAGTGAGACAGACTAGAAGTTCCTAGTGAT
GGCTTATGATAGCAAATGGCCTCATGTCAAATATTTAGATGTAATTTTGTGTAAGAAATACAGAC
TGGATGTACCACCAACTACTACCTGTAATGACAGGCCTGTCCAACACATCTCCCTTTTCCATGAC
TGTGGTAGCCAGCATCGGAAAGAACGCTGATTTAAAGAGGTCGCTTGGGAATTTTATTGACACAG
TACCATTTAATGGGGAGGACAAAATGGGGCAGGGGAGGGAGAAGTTTCTGTCGTTAAAAACAGAT
TTGGAAAGACTGGACTCTAAAGTCTGTTGATTAAAGATGAGCTTTGTCTACTTCAAAAGTTTGTT
TGCTTACCCCTTCAGCCTCCAATTTTTTAAGTGAAAATATAGCTAATAACATGTGAAAAGAATAG
AAGCTAAGGTTTAGATAAATATTGAGCAGATCTATAGGAAGATTGAACCTGAATATTGCCATTAT
GCTTGAC
miRNA-182 target sequence
(SEQ ID NO: 78)
AGTGTGAGTTCTACCATTGCCAAA
miRNA-183 target sequence
(SEQ ID NO: 79)
AGTGAATTCTACCAGTGCCATA
miRNA-194 target sequence
(SEQ ID NO: 107)
TCCACATGGAGTTGCTGTTACA
miRNA-140 target sequence
(SEQ ID NO: 108)
CCGTGGTTCTACCCTGTGGTA
miRNA-18a target sequence
(SEQ ID NO: 109)
CTATCTGCACTAGATGCACCTTA
miRNA-99a target sequence
(SEQ ID NO: 110)
CACAAGATCGGATCTACGGGTT
miRNA-30b target sequence
(SEQ ID NO: 111)
CTGAGTGTAGGATGTTTACA
miRNA-15a target sequence
(SEQ ID NO: 112)
CACAAACCATTATGTGCTGCTA

Internal Ribosome Entry Sites (IRES)

In some embodiments, a construct encoding a connexin 26 protein can include an internal ribosome entry site (IRES). An IRES forms a complex secondary structure that allows translation initiation to occur from any position with an mRNA immediately downstream from where the IRES is located (see, e.g., Pelletier and Sonenberg, Mol. Cell. Biol. 8(3):1103-1112, 1988).

There are several IRES sequences known to those in skilled in the art, including those from, e.g., foot and mouth disease virus (FMDV), encephalomyocarditis virus (EMCV), human rhinovirus (HRV), cricket paralysis virus, human immunodeficiency virus (HIV), hepatitis A virus (HAV), hepatitis C virus (HCV), and poliovirus (PV). See e.g., Alberts, Molecular Biology of the Cell, Garland Science, 2002; and Hellen et al., Genes Dev. 15(13):1593-612, 2001, each of which is incorporated in its entirety herein by reference.

In some embodiments, the IRES sequence that is incorporated into a construct that encodes a connexin 26 protein, or a C-terminal portion of a connexin 26 protein is the foot and mouth disease virus (FMDV) 2A sequence. The Foot and Mouth Disease Virus 2A sequence is a small peptide (approximately 18 amino acids in length) that has been shown to mediate the cleavage of polyproteins (Ryan, M D et al., EMBO 4:928-933, 1994; Mattion et al., J Virology 70:8124-8127, 1996; Furler et al., Gene Therapy 8:864-873, 2001; and Halpin et al., Plant Journal 4:453-459, 1999, each of which is incorporated in its entirety herein by reference). The cleavage activity of the 2A sequence has previously been demonstrated in artificial systems including plasmids and gene therapy constructs (AAV and retroviruses) (Ryan et al., EMBO 4:928-933, 1994; Mattion et al., J Virology 70:8124-8127, 1996; Furler et al., Gene Therapy 8:864-873, 2001; and Halpin et al., Plant Journal 4:453-459, 1999; de Felipe et al., Gene Therapy 6:198-208, 1999; de Felipe et al., Human Gene Therapy I I: 1921-1931, 2000; and Klump et al., Gene Therapy 8:811-817, 2001, each of which is incorporated in its entirety herein by reference).

An IRES can be utilized in an AAV construct. In some embodiments, a construct encoding the C-terminal portion of the connexin 26 protein can include a polynucleotide internal ribosome entry site (IRES). In some embodiments, an IRES can be part of a composition comprising more than one construct. In some embodiments, an IRES is used to produce more than one polypeptide from a single gene transcript.

Splice Sites

In some embodiments, any of the constructs provided herein can include splice donor and/or splice acceptor sequences, which are functional during RNA processing occurring during transcription. In some embodiments, splice sites are involved in trans-splicing.

Exemplary splice donor intron
(SEQ ID NO: SEQ ID NO: 23)
GTAAGTATCAAGGTTACAAGACAGGTTTAAGGAGACCAATAGAAACTGGG
CTTGTCGAGACAGAGAAGACTCTTGCGTTTCT
Exemplary splice acceptor intron
(SEQ ID NO: SEQ ID NO: 24)
GATAGGCACCTATTGGTCTTACTGACATCCACTTTGCCTTTCTCTCCACA
G

Polyadenylation Sequences

In some embodiments, a construct provided herein can include a polyadenylation (poly(A)) signal sequence. Most nascent eukaryotic mRNAs possess a poly(A) tail at their 3′ end, which is added during a complex process that includes cleavage of the primary transcript and a coupled polyadenylation reaction driven by the poly(A) signal sequence (see, e.g., Proudfoot et al., Cell 108:501-512, 2002, which is incorporated herein by reference in its entirety). A poly(A) tail confers mRNA stability and transferability (Molecular Biology of the Cell, Third Edition by B. Alberts et al., Garland Publishing, 1994, which is incorporated herein by reference in its entirety). In some embodiments, a poly(A) signal sequence is positioned 3′ to the coding sequence.

As used herein, “polyadenylation” refers to the covalent linkage of a polyadenylyl moiety, or its modified variant, to a messenger RNA molecule. In eukaryotic organisms, most messenger RNA (mRNA) molecules are polyadenylated at the 3′ end. A 3′ poly(A) tail is a long sequence of adenine nucleotides (e.g., 50, 60, 70, 100, 200, 500, 1000, 2000, 3000, 4000, or 5000) added to the pre-mRNA through the action of an enzyme, polyadenylate polymerase. In some embodiments, a poly(A) tail is added onto transcripts that contain a specific sequence, e.g., a polyadenylation (or poly(A)) signal. A poly(A) tail and associated proteins aid in protecting mRNA from degradation by exonucleases. Polyadenylation also plays a role in transcription termination, export of the mRNA from the nucleus, and translation. Polyadenylation typically occurs in the nucleus immediately after transcription of DNA into RNA, but also can occur later in the cytoplasm. After transcription has been terminated, an mRNA chain is cleaved through the action of an endonuclease complex associated with RNA polymerase. A cleavage site is usually characterized by the presence of the base sequence AAUAAA near the cleavage site. After the mRNA has been cleaved, adenosine residues are added to the free 3′ end at the cleavage site.

As used herein, a “poly(A) signal sequence” or “polyadenylation signal sequence” is a sequence that triggers the endonuclease cleavage of an mRNA and the addition of a series of adenosines to the 3′ end of the cleaved mRNA.

There are several poly(A) signal sequences that can be used, including those derived from bovine growth hormone (bGH) (Woychik et al., Proc. Natl. Acad Sci. US.A. 81(13):3944-3948, 1984; U.S. Pat. No. 5,122,458, each of which is incorporated herein by reference in its entirety), mouse-β-globin, mouse-α-globin (Orkin et al., EMBO J 4(2):453-456, 1985; Thein et al., Blood 71(2):313-319, 1988, each of which is incorporated herein by reference in its entirety), human collagen, polyoma virus (Batt et al., Mol. Cell Biol. 15(9):4783-4790, 1995, which is incorporated herein by reference in its entirety), the Herpes simplex virus thymidine kinase gene (HSV TK), IgG heavy-chain gene polyadenylation signal (US 2006/0040354, which is incorporated herein by reference in its entirety), human growth hormone (hGH) (Szymanski et al., Mol. Therapy 15(7):1340-1347, 2007, which is incorporated herein by reference in its entirety), the group comprising a SV40 poly(A) site, such as the SV40 late and early poly(A) site (Schek et al., Mol. Cell Biol. 12(12):5386-5393, 1992, which is incorporated herein by reference in its entirety).

The poly(A) signal sequence can be AATAAA. The AATAAA sequence may be substituted with other hexanucleotide sequences with homology to AATAAA and that are capable of signaling polyadenylation, including ATTAAA, AGTAAA, CATAAA, TATAAA, GATAAA, ACTAAA, AATATA, AAGAAA, AATAAT, AAAAAA, AATGAA, AATCAA, AACAAA, AATCAA, AATAAC, AATAGA, AATTAA, or AATAAG (see, e.g., WO 06/12414, which is incorporated herein by reference in its entirety).

In some embodiments, a poly(A) signal sequence can be a synthetic polyadenylation site (see, e.g., the pCl-neo expression construct of Promega that is based on Levitt el al., Genes Dev. 3(7):1019-1025, 1989, which is incorporated herein by reference in its entirety). In some embodiments, a poly(A) signal sequence is the polyadenylation signal of soluble neuropilin-1 (sNRP) (AAATAAAATACGAAATG) (see, e.g., WO 05/073384, which is incorporated herein by reference in its entirety). In some embodiments, a poly(A) signal sequence comprises or consists of the SV40 poly(A) site. In some embodiments, a poly(A) signal comprises or consists of SEQ ID NO: 25. In some embodiments, a poly(A) signal sequence comprises or consists of bGHpA. In some embodiments, a poly(A) signal comprises or consists of SEQ ID NO: 26. Additional examples of poly(A) signal sequences are known in the art. In some embodiments, a poly(A) sequence is at least 85%, at least 90%, at least 95%, at least 96%, at least 97%, at least 98% at least 99%, or 100% identical to the poly(A) sequence represented by SEQ ID NO: 25.

Exemplary bGH poly(A) signal sequence
(SEQ ID NO: 25)
CTGTGCCTTCTAGTTGCCAGCCATCTGTTGTTTGCCCCTCCCCCGTGCCT
TCCTTGACCCTGGAAGGTGCCACTCCCACTGTCCTTTCCTAATAAAATGA
GGAAATTGCATCGCATTGTCTGAGTAGGTGTCATTCTATTCTGGGGGGTG
GGGTGGGGCAGGACAGCAAGGGGGAGGATTGGGAAGACAATAGCAGGCAT
GCTGGGGATGCGGTGGGCTCTATGG
Exemplary SV40 poly(A) signal sequence
(SEQ ID NO: 26)
AACTTGTTTATTGCAGCTTATAATGGTTACAAATAAAGCAATAGCATCAC
AAATTTCACAAATAAAGCATTTTTTTCACTGCATTCTAGTTGTGGTTTGT
CCAAACTCATCAATGTATCTTA

Additional Sequences

In some embodiments, constructs of the present disclosure may include one or more filler sequences. In some embodiments, filler sequences may function as regulatory elements, altering construct expression. In some such embodiments, filler sequences may not be fully removed prior to manufacturing for administration to a subject. In some embodiments, filler sequences may have functional roles including as linker sequences, as regulatory regions, or as stabilizing regions. As will be appreciated by those skilled in the art, filler sequences may vary significantly in primary sequence while retaining their desired function. In some embodiments, constructs may contain any combination of filler sequences, exemplary filler sequences which may function as regulatory sequences are represented by SEQ ID NO: 27, or 28.

In some embodiments, constructs of the present disclosure may comprise a T2A element or sequence. In some embodiments, constructs of the present disclosure may include one or more cloning sites. In some such embodiments, cloning sites may not be fully removed prior to manufacturing for administration to a subject. In some embodiments, cloning sites may have functional roles including as linker sequences, portions of a Kozak site, or as sites encoding a stop codon. As will be appreciated by those skilled in the art, cloning sites may vary significantly in primary sequence while retaining their desired function. In some embodiments, constructs may contain any combination of cloning sites, exemplary cloning sites are represented by SEQ ID NO: 29, 30, 31, 32, 33, 34, 35, 36, 37, or 92. In some embodiments, constructs may contain additional cloning sites less than five nucleotides in length.

Exemplary Regulatory sequence C3
(SEQ ID NO: 27)
CTTCTTCTGGAGTCTTTTCTGGAATAATTCTGGGAGTGGGCTCAGCCTGCGGGAGAGTAACATTT
TTATAACTTGATAGATGTAGCTGAGATGCCTCCCAGAGGGGAGACCCGCCTCTCCTCCGGCAGCT
GTGCACGTAGGCTTGTTCCCAGCAGCCTGGCCAGGGTGGTCCACCTGGTGTTTCTCATCTTCTTT
CCCCGGAGCGCTGACTCCTGCGCGTCCTCTTGGAAGACTCTTGACAGGACGGGTGTTTTATGGGT
GTGATTCAGTGTCCTCTTGCATCAGTTCAATGTGGTGGTGTTCAATCAACCCTTGTAGCGTTAGC
AAAATTTGCTCAAGTCATTCCGCAGGAATGTCTGTGTCTTGCTTCCAAGAAAGCTTGTAAGTGCC
GGCAACAGGCCAAGCAGCTCACAAACCTGACCACAAGCCTGTGAGTAATTGTGGGGCAGCACTTA
GCAGTCTTTTATTTTCGACTTATTAAAGTCTCATCTTGGCCTCACCTTCTCCCTGGAAGGTGGCG
TGGGTGGGAACCACTGGGTCAGATCTTTTTCACCCTTGCCGTGGAGCCAGTTTCCTGTTGCATGT
GGGGGAAGCAACATGTGGTGAAGAGTATAGAAAACGAAAACATGTGGGTACAGTATGTATAAGTG
GAGGGAACAAACTCATAATTCCAACTAGTTTCTCATGAGAGACTCATGAATCATTGTGGTAGTTC
TCAATATAAACTTAATCTAGGCCGGATGTGGTGGCTCACACCTGTAATCTCAGCACTCTGGGTGG
ATCACTTGAGGTCAGGAGTTTGAGACCAGTCTGACCAACATGGAGAAACCCCATCGCTACTAAAA
ATACAAAATTATCCAGATGTGGTGGCTCACACCTGTAATCCCAGCACTTTGGGAGGCTGAGGCGG
GTGGATCACTTGAGGTCAGGAGTTTGAGACCAGCCTGACCAACATGGAGAAACTGTGTCTCTACT
AAAAATACAAAATTAGCTGGGCGTGGTGACGCATGCCTGTAATCCCAGCTATTTGGAGGCCGAAG
CAGG
Exemplary Regulatory sequence D7
(SEQ ID NO: 28)
CTTCTTCTGGAGTCTTTTCTGGAATAATTCTGGGAGTGGGCTCAGCCTGCGGGAGAGTAACATTT
TTATAACTTGATAGATGTAGCTGAGATGCCTCCCAGAGGGGAGACCCGCCTCTCCTCCGGCAGCT
GTGCACGTAGGCTTGTTCCCAGCAGCCTGGCCAGGGTGGTCCACCTGGTGTTTCTCATCTTCTTT
CCCCGGAGCGCTGACTCCTGCGCGTCCTCTTGGAAGACTCTTGACAGGACGGGTGTTTTATGGGT
GTGATTCAGTGTCCTCTTGCATCAGTTCAATGTGGTGGTGTTCAATCAACCCTTGTAGCGTTAGC
AAAATTTGCTCAAGTCATTCCGCAGGAATGTCTGTGTCTTGCTTCCAAGAAAGCTTGTAAGTGCC
GGCAACAGGCCAAGCAGCTCACAAACCTGACCACAAGCCTGTGAGTAATTGTGGGGCAGCACTTA
GCAGTCTTTTATTTTCGACTTATTAAAGTCTCATCTTGGCCTCACCTTCTCCCTGGAAGGTGGCG
TGGGTGGGAACCACTGGGTCAGATCTTTTTCACCCTTGCCGTGGAGCCAGTTTCCTGTTGCATGT
GGGGGAAGCAACATGTGGTGAAGAGTATAGAAAACGAAAACATGTGGGTACAGTATGTATAAGTG
GAGGGAACAAACTCATAATTCCAACTAGTTTCTCATGAGAGACTCATGAATCATTGTGGTAGTTC
TCAATATAAACTTAATCTAGGCCGGATGTGGTGGCTCACACCTGTAATCTCAGCACTCTGGGTGG
ATCACTTGAGGTCAGGAGTTTGAGACCAGTCTGACCAACATGGAGAAACCCCATCGCTACTAAAA
ATACAAAATTATCCAGATGTGGTGGCTCACACCTGTAATCCCAGCACTTTGGGAGGCTGAGGCGG
GTGGATCACTTGAGGTCAGGAGTTTGAGACCAGCCTGACCAACATGGAGAAACTGTGTCTCTACT
AAAAATACAAAATTAGCTGGGCGTGGTGACGCATGCCTGTAATCCCAGCTATTTGGAGGCCGAAG
CAGG
Exemplary cloning site A
(SEQ ID NO: 29)
TTGTCGACGCGGCCGCACGCGT
Exemplary cloning site B
(SEQ ID NO: 30)
CTCCTGGGCAACGTGCTGGTTATTGTGACCGGTGCCACC
Exemplary cloning site C
(SEQ ID NO: 31)
TAAGAGCTCGCTGATCAGCCTCGA
Exemplary cloning site D
(SEQ ID NO: 32)
AAGCTTGAATTCAGCTGACGTGCCTCGGACCGCCTAGG
Exemplary cloning site E
(SEQ ID NO: 33)
TAAGAGCTC
Exemplary cloning site F
(SEQ ID NO: 34)
GCTGATCAGCCTCGA
Exemplary cloning site G
(SEQ ID NO: 35)
GGCATTCCGGTACTGTTGGTAAAGCCACCAGCAAACCGCCCAGAGTAGAAGACCGGTGGCCACC
Exemplary cloning site H
(SEQ ID NO: 36)
AAGCTTGAATTC
Exemplary cloning site I
(SEQ ID NO: 37)
AGCTGACGTGCCTCGGACCGCCTAGG
Exemplary cloning site J
(SEQ ID NO: 70)
GCGGCCGCACGCGT
Exemplary cloning site K
(SEQ ID NO: 71)
GCGGCCGCACGCGTGGT
Exemplary cloning site L
(SEQ ID NO: 72)
CTCCTGGGCAACGTGCTGGTTATTGTGACCGGT
Exemplary cloning site M
(SEQ ID NO: 73)
CGCTAGCCACC
Exemplary cloning site N
(SEQ ID NO: 74)
ACCGGTCGCTAGCCACC
Exemplary cloning site O
(SEQ ID NO: 75)
GAGCTCGCTGATCAGCCTCGA
Exemplary cloning site P
(SEQ ID NO: 76)
AAGCTTGAATTCAGCTGACGTGCCTCGGACCGCT
Exemplary cloning site Q
(SEQ ID NO: 92)
CTCACCGGT
Exemplary linker sequence
(SEQ ID NO: 77)
GGATCCCGGGCT

### Destabilization Domains

In some embodiments, any of the constructs provided herein can optionally include a sequence encoding a destabilizing domain (“a destabilizing sequence”) for temporal control of protein expression. Non-limiting examples of destabilizing sequences include sequences encoding a FK506 sequence, a dihydrofolate reductase (DHFR) sequence, or other exemplary destabilizing sequences.

In the absence of a stabilizing ligand, a protein sequence operatively linked to a destabilizing sequence is degraded by ubiquitination. In contrast, in the presence of a stabilizing ligand, protein degradation is inhibited, thereby allowing the protein sequence operatively linked to the destabilizing sequence to be actively expressed. As a positive control for stabilization of protein expression, protein expression can be detected by conventional means, including enzymatic, radiographic, colorimetric, fluorescence, or other spectrographic assays; fluorescent activating cell sorting (FACS) assays; immunological assays (e.g., enzyme linked immunosorbent assay (ELISA), radioimmunoassay (RIA), and immunohistochemistry).

Additional examples of destabilizing sequences are known in the art. In some embodiments, the destabilizing sequence is a FK506- and rapamycin-binding protein (FKBP12) sequence, and the stabilizing ligand is Shield-1 (Shld1) (Banaszynski et al., (2012) Cell 126(5): 995-1004, which is incorporated in its entirety herein by reference). In some embodiments, a destabilizing sequence is a DHFR sequence, and a stabilizing ligand is trimethoprim (TMP) (Iwamoto et al., (2010) Chem Biol 17:981-988, which is incorporated in its entirety herein by reference).

In some embodiments, a destabilizing sequence is a FKBP12 sequence, and a presence of an AAV construct carrying the FKBP12 gene in a subject cell (e.g., a supporting cochlear outer hair cell) is detected by western blotting. In some embodiments, a destabilizing sequence can be used to verify the temporally-specific activity of any of the AAV constructs described herein.

In some embodiments, a destabilizing domain may be a target site for an inhibitory nucleic acid. In some embodiments, a destabilizing domain is a non-endogenous sequence that has been introduced into a regulatory region of an RNA molecule. In some embodiments, a destabilizing domain may permit temporal and/or spatial control of an mRNA molecule. In some embodiments, a destabilizing domain may be a target of endogenously expressed inhibitory nucleic acid molecules. In some embodiments, a destabilizing domain may be an miRNA regulatory target site and/or sites (miRTS) as described herein. In some embodiments, a destabilizing domain is represented by SEQ ID NO: 78. In some embodiments, a destabilizing domain is represented by SEQ ID NO: 79. In some embodiments, a destabilizing domain is represented by SEQ ID NO: 80.

Exemplary mRNA destabilizing domain sequence
(SEQ ID NO: 78)
AGTGTGAGTTCTACCATTGCCAAA
Exemplary mRNA destabilizing domain sequence
(SEQ ID NO: 79)
AGTGAATTCTACCAGTGCCATA
Exemplary mRNA destabilizing domain Sequence
(SEQ ID NO: 80)
GAGCTCAGTGTGAGTTCTACCATTGCCAAACTCGAGCAGTGAATTCTACC
AGTGCCATAGGATCCAGTGTGAGTTCTACCATTGCCAAAGGTACCCAGTG
AATTCTACCAGTGCCATAGTTAAC
Exemplary DHFR destabilizing amino acid sequence
(SEQ ID NO: 38)
MISLIAALAVDYVIGMENAMPWNLPADLAWFKRNTLNKPVIMGRHTWESI
GRPLPGRKNIILSSQPSTDDRVTWVKSVDEAIAACGDVPEIMVIGGGRVI
EQFLPKAQKLYLTHIDAEVEGDTHFPDYEPDDWESVFSEFHDADAQNSHS
YCFEILERR
Exemplary DHFR destabilizing nucleotide sequence
(SEQ ID NO: 39)
GGTACCATCAGTCTGATTGCGGCGTTAGCGGTAGATTACGTTATCGGCAT
GGAAAACGCCATGCCGTGGAACCTGCCTGCCGATCTCGCCTGGTTTAAAC
GCAACACCTTAAATAAACCCGTGATTATGGGCCGCCATACCTGGGAATCA
ATCGGTCGTCCGTTGCCAGGACGCAAAAATATTATCCTCAGCAGTCAACC
GAGTACGGACGATCGCGTAACGTGGGTGAAGTCGGTGGATGAAGCCATCG
CGGCGTGTGGTGACGTACCAGAAATCATGGTGATTGGCGGCGGTCGCGTT
ATTGAACAGTTCTTGCCAAAAGCGCAAAAACTGTATCTGACGCATATCGA
CGCAGAAGTGGAAGGCGACACCCATTTCCCGGATTACGAGCCGGATGACT
GGGAATCGGTATTCAGCGAATTCCACGATGCTGATGCGCAGAACTCTCAC
AGCTATTGCTTTGAGATTCTGGAGCGGCGATAA
Exemplary destabilizing domain
(SEQ ID NO: 40)
ATCAGTCTGATTGCGGCGTTAGCGGTAGATTACGTTATCGGCATGGAAAA
CGCCATGCCGTGGAACCTGCCTGCCGATCTCGCCTGGTTTAAACGCAACA
CCTTAAATAAACCCGTGATTATGGGCCGCCATACCTGGGAATCAATCGGT
CGTCCGTTGCCAGGACGCAAAAATATTATCCTCAGCAGTCAACCGAGTAC
GGACGATCGCGTAACGTGGGTGAAGTCGGTGGATGAAGCCATCGCGGCGT
GTGGTGACGTACCAGAAATCATGGTGATTGGCGGCGGTCGCGTTATTGAA
CAGTTCTTGCCAAAAGCGCAAAAACTGTATCTGACGCATATCGACGCAGA
AGTGGAAGGCGACACCCATTTCCCGGATTACGAGCCGGATGACTGGGAAT
CGGTATTCAGCGAATTCCACGATGCTGATGCGCAGAACTCTCACAGCTAT
TGCTTTGAGATTCTGGAGCGGCGA
Exemplary FKBP12 destabilizing peptide amino acid
sequence
(SEQ ID NO: 41)
MGVEKQVIRPGNGPKPAPGQTVTVHCTGFGKDGDLSQKFWSTKDEGQKPF
SFQIGKGAVIKGWDEGVIGMQIGEVARLRCSSDYAYGAGGFPAWGIQPNS
VLDFEIEVLSVQ

Reporter Sequences or Elements

In some embodiments, constructs provided herein can optionally include a sequence encoding a reporter polypeptide and/or protein (“a reporter sequence”). Non-limiting examples of reporter sequences include DNA sequences encoding: a beta-lactamase, a beta-galactosidase (LacZ), an alkaline phosphatase, a thymidine kinase, a green fluorescent protein (GFP), a red fluorescent protein, an mCherry fluorescent protein, a yellow fluorescent protein, a chloramphenicol acetyltransferase (CAT), and a luciferase. Additional examples of reporter sequences are known in the art. When associated with control elements which drive their expression, the reporter sequence can provide signals detectable by conventional means, including enzymatic, radiographic, colorimetric, fluorescence, or other spectrographic assays; fluorescent activating cell sorting (FACS) assays; immunological assays (e.g., enzyme linked immunosorbent assay (ELISA), radioimmunoassay (RIA), and immunohistochemistry).

In some embodiments, a reporter sequence is the LacZ gene, and the presence of a construct carrying the LacZ gene in a mammalian cell (e.g., a cochlear hair cell) is detected by assays for beta-galactosidase activity. When the reporter is a fluorescent protein (e.g., green fluorescent protein) or luciferase, the presence of a construct carrying the fluorescent protein or luciferase in a mammalian cell (e.g., a cochlear hair cell) may be measured by fluorescent techniques (e.g., fluorescent microscopy or FACS) or light production in a luminometer (e.g., a spectrophotometer or an IVIS imaging instrument). In some embodiments, a reporter sequence can be used to verify the tissue-specific targeting capabilities and tissue-specific promoter regulatory and/or control activity of any of the constructs described herein.

In some embodiments, a reporter sequence is a FLAG tag (e.g., a 3×FLAG tag), and the presence of a construct carrying the FLAG tag in a mammalian cell (e.g., an inner ear cell, e.g., a cochlear hair or supporting cell) is detected by protein binding or detection assays (e.g., Western blots, immunohistochemistry, radioimmunoassay (RIA), mass spectrometry). An exemplary 3×FLAG tag sequence is provided as SEQ ID NO: 42.

Exemplary 3xFLAG tag sequence
(SEQ ID NO: 42)
GGATCCCGGGCTGACTACAAAGACCATGACGGTGATTATAAAGATCATGA
CATCGACTACAAGGATGACGATGACAAG
Exemplary 3xFLAG tag sequence with stop codon
(SEQ ID NO: 81)
GACTACAAAGACCATGACGGTGATTATAAAGATCATGACATCGACTACAA
GGATGACGATGACAAGTAA
Exemplary barcode tag
(SEQ ID NO: 93)
GTGTCACC
Exemplary barcode tag
(SEQ ID NO: 96)
CACAACCT
Exemplary barcode tag
(SEQ ID NO: 99)
CGTGTGTT
Exemplary barcode tag
(SEQ ID NO: 102)
TCGTGGGT
Exemplary barcode tag
(SEQ ID NO: 105)
GCAAACTG

AAV Capsids

The present disclosure provides one or more polynucleotide constructs packaged into an AAV capsid. In some embodiments, an AAV capsid is from or derived from an AAV capsid of an AAV2, 3, 4, 5, 6, 7, 8, 9, 10, rh8, rh10, rh39, rh43 or Anc80 serotype, or one or more hybrids thereof. In some embodiments, an AAV capsid is from an AAV ancestral serotype. In some embodiments, an AAV capsid is an ancestral (Anc) AAV capsid. An Anc capsid is created from a construct sequence that is constructed using evolutionary probabilities and evolutionary modeling to determine a probable ancestral sequence. Thus, an Anc capsid/construct sequence is not known to have existed in nature. For example, in some embodiments, an AAV capsid is an Anc80 capsid (e.g., an Anc80L65 capsid). In some embodiments, an AAV capsid is created using a template nucleotide coding sequence comprising SEQ ID NO: 43. In some embodiments, the capsid comprises a polypeptide represented by SEQ ID NO: 44. In some embodiments, the capsid comprises a polypeptide with at least 85%, at least 90%, at least 95%, at least 96%, at least 97%, at least 98% at least 99%, or 100% identical to the polypeptide represented by SEQ ID NO: 44.

As provided herein, any combination of AAV capsids and AAV constructs (e.g., comprising AAV ITRs) may be used in recombinant AAV (rAAV) particles of the present disclosure. For example, wild-type or variant AAV2 ITRs and Anc80 capsid, wild-type or variant AAV2 ITRs and AAV6 capsid, etc. In some embodiments of the present disclosure, an AAV particle is wholly comprised of AAV2 components (e.g., capsid and ITRs are AAV2 serotype). In some embodiments, an AAV particle is an AAV2/6, AAV2/8 or AAV2/9 particle (e.g., an AAV6, AAV8 or AAV9 capsid with an AAV construct having AAV2 ITRs). In some aspects, an AAV capsid is an Anc80 capsid (e.g., an Anc80L65 capsid). In some embodiments of the present disclosure, an AAV particle is an AAV2/Anc80 particle that comprises an Anc80 capsid (e.g., comprising a polypeptide of SEQ ID NO: 44) that encapsidates an AAV construct with AAV2 ITRs (e.g., SEQ ID NOs: 8 and 9) flanking a portion of a coding sequence, for example, a GJB2 gene or characteristic portion thereof (e.g., SEQ ID NO: 1, 2, 3, 4, 5, or 6). Other AAV particles are known in the art and are described in, e.g., Sharma et al., Brain Res Bull. 2010 Feb. 15; 81(2-3): 273, which is incorporated in its entirety herein by reference. In some embodiments, a capsid sequence is at least 85%, at least 90%, at least 95%, at least 96%, at least 97%, at least 98% at least 99%, or 100% identical to a capsid nucleotide or amino acid sequence represented by SEQ ID NO: 43 or 44, respectively.

Exemplary AAV Anc80 Capsid DNA Sequence
(SEQ ID NO: 43)
ATGGCTGCCGATGGTTATCTTCCAGATTGGCTCGAGGACAACCTCTCTGA
GGGCATTCGCGAGTGGTGGGACTTGAAACCTGGAGCCCCGAAACCCAAAG
CCAACCAGCAAAAGCAGGACGACGGCCGGGGTCTGGTGCTTCCTGGCTAC
AAGTACCTCGGACCCTTCAACGGACTCGACAAGGGGGAGCCCGTCAACGC
GGCGGACGCAGCGGCCCTCGAGCACGACAAGGCCTACGACCAGCAGCTCA
AAGCGGGTGACAATCCGTACCTGCGGTATAACCACGCCGACGCCGAGTTT
CAGGAGCGTCTGCAAGAAGATACGTCTTTTGGGGGCAACCTCGGGCGAGC
AGTCTTCCAGGCCAAGAAGCGGGTTCTCGAACCTCTCGGTCTGGTTGAGG
AAGGCGCTAAGACGGCTCCTGGAAAGAAGAGACCGGTAGAGCAATCACCC
CAGGAACCAGACTCCTCTTCGGGCATCGGCAAGAAAGGCCAGCAGCCCGC
GAAGAAGAGACTCAACTTTGGGCAGACAGGCGACTCAGAGTCAGTGCCCG
ACCCTCAACCACTCGGAGAACCCCCCGCAGCCCCCTCTGGTGTGGGATCT
AATACAATGGCAGCAGGCGGTGGCGCTCCAATGGCAGACAATAACGAAGG
CGCCGACGGAGTGGGTAACGCCTCAGGAAATTGGCATTGCGATTCCACAT
GGCTGGGCGACAGAGTCATCACCACCAGCACCCGAACCTGGGCCCTCCCC
ACCTACAACAACCACCTCTACAAGCAAATCTCCAGCCAATCGGGAGCAAG
CACCAACGACAACACCTACTTCGGCTACAGCACCCCCTGGGGGTATTTTG
ACTTTAACAGATTCCACTGCCACTTCTCACCACGTGACTGGCAGCGACTC
ATCAACAACAACTGGGGATTCCGGCCCAAGAGACTCAACTTCAAGCTCTT
CAACATCCAGGTCAAGGAGGTCACGACGAATGATGGCACCACGACCATCG
CCAATAACCTTACCAGCACGGTTCAGGTCTTTACGGACTCGGAATACCAG
CTCCCGTACGTCCTCGGCTCTGCGCACCAGGGCTGCCTGCCTCCGTTCCC
GGCGGACGTCTTCATGATTCCTCAGTACGGGTACCTGACTCTGAACAATG
GCAGTCAGGCCGTGGGCCGTTCCTCCTTCTACTGCCTGGAGTACTTTCCT
TCTCAAATGCTGAGAACGGGCAACAACTTTGAGTTCAGCTACACGTTTGA
GGACGTGCCTTTTCACAGCAGCTACGCGCACAGCCAAAGCCTGGACCGGC
TGATGAACCCCCTCATCGACCAGTACCTGTACTACCTGTCTCGGACTCAG
ACCACGAGTGGTACCGCAGGAAATCGGACGTTGCAATTTTCTCAGGCCGG
GCCTAGTAGCATGGCGAATCAGGCCAAAAACTGGCTACCCGGGCCCTGCT
ACCGGCAGCAACGCGTCTCCAAGACAGCGAATCAAAATAACAACAGCAAC
TTTGCCTGGACCGGTGCCACCAAGTATCATCTGAATGGCAGAGACTCTCT
GGTAAATCCCGGTCCCGCTATGGCAACCCACAAGGACGACGAAGACAAAT
TTTTTCCGATGAGCGGAGTCTTAATATTTGGGAAACAGGGAGCTGGAAAT
AGCAACGTGGACCTTGACAACGTTATGATAACCAGTGAGGAAGAAATTAA
AACCACCAACCCAGTGGCCACAGAACAGTACGGCACGGTGGCCACTAACC
TGCAATCGTCAAACACCGCTCCTGCTACAGGGACCGTCAACAGTCAAGGA
GCCTTACCTGGCATGGTCTGGCAGAACCGGGACGTGTACCTGCAGGGTCC
TATCTGGGCCAAGATTCCTCACACGGACGGACACTTTCATCCCTCGCCGC
TGATGGGAGGCTTTGGACTGAAACACCCGCCTCCTCAGATCCTGATTAAG
AATACACCTGTTCCCGCGAATCCTCCAACTACCTTCAGTCCAGCTAAGTT
TGCGTCGTTCATCACGCAGTACAGCACCGGACAGGTCAGCGTGGAAATTG
AATGGGAGCTGCAGAAAGAAAACAGCAAACGCTGGAACCCAGAGATTCAA
TACACTTCCAACTACAACAAATCTACAAATGTGGACTTTGCTGTTGACAC
AAATGGCGTTTATTCTGAGCCTCGCCCCATCGGCACCCGTTACCTCACCC
GTAATCTG
Exemplary AAV Anc80 Capsid Amino Acid Sequence
(SEQ ID NO: 44)
MAADGYLPDWLEDNLSEGIREWWDLKPGAPKPKANQQKQDDGRGLVLPGY
KYLGPFNGLDKGEPVNAADAAALEHDKAYDQQLKAGDNPYLRYNHADAEF
QERLQEDTSFGGNLGRAVFQAKKRVLEPLGLVEEGAKTAPGKKRPVEQSP
QEPDSSSGIGKKGQQPAKKRLNFGQTGDSESVPDPQPLGEPPAAPSGVGS
NTMAAGGGAPMADNNEGADGVGNASGNWHCDSTWLGDRVITTSTRTWALP
TYNNHLYKQISSQSGASTNDNTYFGYSTPWGYFDFNRFHCHFSPRDWQRL
INNNWGFRPKRLNFKLFNIQVKEVTTNDGTTTIANNLTSTVQVFTDSEYQ
LPYVLGSAHQGCLPPFPADVFMIPQYGYLTLNNGSQAVGRSSFYCLEYFP
SQMLRTGNNFEFSYTFEDVPFHSSYAHSQSLDRLMNPLIDQYLYYLSRTQ
TTSGTAGNRTLQFSQAGPSSMANQAKNWLPGPCYRQQRVSKTANQNNNSN
FAWTGATKYHLNGRDSLVNPGPAMATHKDDEDKFFPMSGVLIFGKQGAGN
SNVDLDNVMITSEEEIKTTNPVATEQYGTVATNLQSSNTAPATGTVNSQG
ALPGMVWQNRDVYLQGPIWAKIPHTDGHFHPSPLMGGFGLKHPPPQILIK
NTPVPANPPTTFSPAKFASFITQYSTGQVSVEIEWELQKENSKRWNPEIQ
YTSNYNKSTNVDFAVDTNGVYSEPRPIGTRYLTRNL

### Compositions

Among other things, the present disclosure provides compositions. In some embodiments, a composition comprises a construct as described herein. In some embodiments, a composition comprises one or more constructs as described herein. In some embodiments, a composition comprises a plurality of constructs as described herein. In some embodiments, when more than one construct is included in the composition, the constructs are each different.

In some embodiments, a composition comprises an AAV particle as described herein. In some embodiments, a composition comprises one or more AAV particles as described herein. In some embodiments, a composition comprises a plurality of AAV particles. In come embodiments, when more than one AAV particle is included in the composition, the AAV particles are each different.

In some embodiments, a composition comprises connexin 26 protein. In some embodiments, a composition comprises a cell.

In some embodiments, a composition is or comprises a pharmaceutical composition.

Dosing and Volume of Administration

In some embodiments, a composition disclosed herein, e.g., one or a plurality of AAV vectors disclosed herein, is administered as a single dose or as a plurality of doses.

In some embodiments, a composition disclosed herein is administered as a single dose. In some embodiments, a composition disclosed herein is administered as a plurality of doses, e.g., 2, 3, 4, 5, 6, 7, 8, 9 or 10 doses.

In some embodiments, a composition disclosed herein (e.g., a composition comprising one or a plurality of rAAV constructs disclosed herein) is administered at a volume of about 0.01 mL, about 0.02 mL, about 0.03 mL, about 0.04 mL, about 0.05 mL, about 0.06 mL, about 0.07 mL, about 0.08 mL, about 0.09 mL, about 1.00 mL, about 1.10 mL, about 1.20 mL, about 1.30 mL, about 1.40 mL, about 1.50 mL, about 1.60 mL, about 1.70 mL, about 1.80 mL, about 1.90 mL, or about 2.00 mL. In some embodiments, a composition disclosed herein is administered at a volume of about 0.01 mL. In some embodiments, a composition disclosed herein is administered at a volume of about 0.02 mL. In some embodiments, a composition disclosed herein is administered at a volume of about 0.03 mL. In some embodiments, a composition disclosed herein is administered at a volume of about 0.04 mL. In some embodiments, a composition disclosed herein is administered at a volume of about 0.05 mL. In some embodiments, a composition disclosed herein is administered at a volume of about 0.06 mL. In some embodiments, a composition disclosed herein is administered at a volume of about 0.07 mL. In some embodiments, a composition disclosed herein is administered at a volume of about 0.08 mL. In some embodiments, a composition disclosed herein is administered at a volume of about 0.09 mL. In some embodiments, a composition disclosed herein is administered at a volume of about 1.00 mL. In some embodiments, a composition disclosed herein is administered at a volume of about 1.10 mL. In some embodiments, a composition disclosed herein is administered at a volume of about 1.20 mL. In some embodiments, a composition disclosed herein is administered at a volume of about 1.30 mL. In some embodiments, a composition disclosed herein is administered at a volume of about 1.40 mL. In some embodiments, a composition disclosed herein is administered at a volume of about 1.50 mL. In some embodiments, a composition disclosed herein is administered at a volume of about 1.60 mL. In some embodiments, a composition disclosed herein is administered at a volume of about 1.70 mL. In some embodiments, a composition disclosed herein is administered at a volume of about 1.80 mL. In some embodiments, a composition disclosed herein is administered at a volume of about 1.90 mL. In some embodiments, a composition disclosed herein is administered at a volume of about 2.00 mL.

In some embodiments, a composition disclosed herein (e.g., a composition comprising one or a plurality of rAAV constructs disclosed herein) is administered at a volume of about 0.01 to 2.00 mL, about 0.02 to 1.90 mL, about 0.03 to 1.8 mL, about 0.04 to 1.70 mL, about 0.05 to 1.60 mL, about 0.06 to 1.50 mL, about 0.06 to 1.40 mL, about 0.07 to 1.30 mL, about 0.08 to 1.20 mL, or about 0.09 to 1.10 mL. In some embodiments a composition disclosed herein (e.g., a composition comprising one or a plurality of rAAV constructs disclosed herein) is administered at a volume of about 0.01 to 2.00 mL, about 0.02 to 2.00 mL, about 0.03 to 2.00 mL, about 0.04 to 2.00 mL, about 0.05 to 2.00 mL, about 0.06 to 2.00 mL, about 0.07 to 2.00 mL, about 0.08 to 2.00 mL, about 0.09 to 2.00 mL, about 0.01 to 1.90 mL, about 0.01 to 1.80 mL, about 0.01 to 1.70 mL, about 0.01 to 1.60 mL, about 0.01 to 1.50 mL, about 0.01 to 1.40 mL, about 0.01 to 1.30 mL, about 0.01 to 1.20 mL, about 0.01 to 1.10 mL, about 0.01 to 1.00 mL, about 0.01 to 0.09 mL.

In some embodiments, a dosing regimen comprises delivery in a volume of at least 0.01 mL, at least 0.02 mL, at least 0.03 mL, at least 0.04 mL, at least 0.05 mL, at least 0.06 mL, at least 0.07 mL, at least 0.08 mL, at least 0.09 mL, at least 0.10 mL, at least 0.11 mL, at least 0.12 mL, at least 0.13 mL, at least 0.14 mL, at least 0.15 mL, at least 0.16 mL, at least 0.17 mL, at least 0.18 mL, at least 0.19 mL, or at least 0.20 mL per cochlea. In some embodiments, a dosing regimen comprises delivery in a volume of at most 0.30 mL, at most 0.25 mL, at most 0.20 mL, at most 0.15 mL, at most 0.14 mL, at most 0.13 mL, at most 0.12 mL, at most 0.11 mL, at most 0.10 mL, at most 0.09 mL, at most 0.08 mL, at most 0.07 mL, at most 0.06 mL, or at most 0.05 mL per cochlea. In some embodiments, the dosing regimen comprises delivery in a volume of about 0.05 mL, about 0.06 mL, about 0.07 mL, about 0.08 mL, about 0.09 mL, about 0.10 mL, about 0.11 mL, about 0.12 mL, about 0.13 mL, about 0.14 mL, or about 0.15 mL per cochlea, depending on the population.

Single A AV Construct Compositions

In some embodiments, the present disclosure provides compositions or systems comprising AAV particles comprised of a single construct. In some such embodiments, a single construct may deliver a polynucleotide that encodes a functional (e.g., wild-type or otherwise functional, e.g., codon optimized) copy of a GJB2 gene. In some embodiments, a construct is or comprises an rAAV construct. In some embodiments described herein, a single rAAV construct is capable of expressing a full-length GJB2 messenger RNA or a characteristic protein thereof in a target cell (e.g., an inner ear cell). In some embodiments, a single construct (e.g., any of the constructs described herein) can include a sequence encoding a functional connexin 26 protein (e.g., any construct that generates functional connexin 26 protein). In some embodiments, a single construct (e.g., any of the constructs described herein) can include a sequence encoding a functional connexin 26 protein (e.g., any construct that generates functional connexin 26 protein) and optionally additional polypeptide sequences (e.g., regulatory sequences, and/or reporter sequences).

In some embodiments, a single construct composition or system may comprise any or all of the exemplary construct components described herein. In some aspects, the, the construct comprises a nucleic acid sequence with at least 85%, at least 90%, at least 95%, at least 96%, at least 97%, at least 98% at least 99%, or 100% identity to SEQ ID NO: 45. In some embodiments, an exemplary single construct is represented by SEQ ID NO: 45. In some aspects, the, the construct comprises a nucleic acid sequence with at least 85%, at least 90%, at least 95%, at least 96%, at least 97%, at least 98% at least 99%, or 100% identity to SEQ ID NO: 46. In some embodiments, an exemplary single construct is represented by SEQ ID NO: 46. In some aspects, the, the construct comprises a nucleic acid sequence with at least 85%, at least 90%, at least 95%, at least 96%, at least 97%, at least 98% at least 99%, or 100% identity to SEQ ID NO: 47. In some embodiments, an exemplary single construct is represented by SEQ ID NO: 47. In some aspects, the, the construct comprises a nucleic acid sequence with at least 85%, at least 90%, at least 95%, at least 96%, at least 97%, at least 98% at least 99%, or 100% identity to SEQ ID NO: 48. In some embodiments, an exemplary single construct is represented by SEQ ID NO: 48. In some aspects, the, the construct comprises a nucleic acid sequence with at least 85%, at least 90%, at least 95%, at least 96%, at least 97%, at least 98% at least 99%, or 100% identity to SEQ ID NO: 49. In some embodiments, an exemplary single construct is represented by SEQ ID NO: 49. In some aspects, the, the construct comprises a nucleic acid sequence with at least 85%, at least 90%, at least 95%, at least 96%, at least 97%, at least 98% at least 99%, or 100% identity to SEQ ID NO: 50. In some embodiments, an exemplary single construct is represented by SEQ ID NO: 50. In some aspects, the, the construct comprises a nucleic acid sequence with at least 85%, at least 90%, at least 95%, at least 96%, at least 97%, at least 98% at least 99%, or 100% identity to SEQ ID NO: 51. In some embodiments, an exemplary single construct is represented by SEQ ID NO: 51. In some aspects, the, the construct comprises a nucleic acid sequence with at least 85%, at least 90%, at least 95%, at least 96%, at least 97%, at least 98% at least 99%, or 100% identity to SEQ ID NO: 82. In some embodiments, an exemplary single construct is represented by SEQ ID NO: 82. In some aspects, the, the construct comprises a nucleic acid sequence with at least 85%, at least 90%, at least 95%, at least 96%, at least 97%, at least 98% at least 99%, or 100% identity to SEQ ID NO: 83. In some embodiments, an exemplary single construct is represented by SEQ ID NO: 83. In some aspects, the, the construct comprises a nucleic acid sequence with at least 85%, at least 90%, at least 95%, at least 96%, at least 97%, at least 98% at least 99%, or 100% identity to SEQ ID NO: 84. In some embodiments, an exemplary single construct is represented by SEQ ID NO: 84. In some aspects, the, the construct comprises a nucleic acid sequence with at least 85%, at least 90%, at least 95%, at least 96%, at least 97%, at least 98% at least 99%, or 100% identity to SEQ ID NO: 85. In some embodiments, an exemplary single construct is represented by SEQ ID NO: 85. In some aspects, the, the construct comprises a nucleic acid sequence with at least 85%, at least 90%, at least 95%, at least 96%, at least 97%, at least 98% at least 99%, or 100% identity to SEQ ID NO: 86. In some embodiments, an exemplary single construct is represented by SEQ ID NO: 86. In some aspects, the, the construct comprises a nucleic acid sequence with at least 85%, at least 90%, at least 95%, at least 96%, at least 97%, at least 98% at least 99%, or 100% identity to SEQ ID NO: 87. In some embodiments, an exemplary single construct is represented by SEQ ID NO: 87. In some aspects, the, the construct comprises a nucleic acid sequence with at least 85%, at least 90%, at least 95%, at least 96%, at least 97%, at least 98% at least 99%, or 100% identity to SEQ ID NO: 88. In some embodiments, an exemplary single construct is represented by SEQ ID NO: 88. In some aspects, the construct comprises the nucleic acid sequence of SEQ ID NO: 94.

In some aspects, the construct comprises the nucleic acid sequence of SEQ ID NO: 97. In some aspects, the, the construct comprises a nucleic acid sequence with at least 85%, at least 90%, at least 95%, at least 96%, at least 97%, at least 98% at least 99%, or 100% identity to SEQ ID NO: 97. In some aspects, the construct comprises the nucleic acid sequence of SEQ ID NO: 100. In some aspects, the, the construct comprises a nucleic acid sequence with at least 85%, at least 90%, at least 95%, at least 96%, at least 97%, at least 98% at least 99%, or 100% identity to SEQ ID NO: 100. In some aspects, the construct comprises the nucleic acid sequence of SEQ ID NO: 103. In some aspects, the, the construct comprises a nucleic acid sequence with at least 85%, at least 90%, at least 95%, at least 96%, at least 97%, at least 98% at least 99%, or 100% identity to SEQ ID NO: 103. In some aspects, the construct comprises the nucleic acid sequence of SEQ ID NO: 106. In some aspects, the, the construct comprises a nucleic acid sequence with at least 85%, at least 90%, at least 95%, at least 96%, at least 97%, at least 98% at least 99%, or 100% identity to SEQ ID NO: 106.One skilled in the art would recognize that constructs may undergo additional modifications including codon-optimization, introduction of novel but functionally equivalent (e.g., silent mutations), addition of reporter sequences, and/or other routine modification.

In some embodiments, an exemplary rAAVAnc80 particle comprises a construct represented by SEQ ID NO: 45.

In one embodiment, an exemplary construct comprises: a 5′ ITR exemplified by SEQ ID NO: 8, optionally a cloning site exemplified by SEQ ID NO: 29, a CMV enhancer exemplified by SEQ ID NO: 18, a CBA promoter exemplified by SEQ ID NO: 11, a chimeric intron exemplified by SEQ ID NO: 19, optionally a cloning site exemplified by SEQ ID NO: 30, a GJB2 coding region exemplified by SEQ ID NO: 1, optionally a cloning site exemplified by SEQ ID NO: 31, a poly(A) site exemplified by SEQ ID NO: 25, optionally a cloning site exemplified by SEQ ID NO: 32, and a 3′ ITR exemplified by SEQ ID NO: 9.

In some embodiments, an exemplary rAAVAnc80 particle comprises a construct represented by SEQ ID NO: 46.

In one embodiment, an exemplary construct comprises: a 5′ ITR exemplified by SEQ ID NO: 8, optionally a cloning site exemplified by SEQ ID NO: 29, a CMV enhancer exemplified by SEQ ID NO: 18, a CBA promoter exemplified by SEQ ID NO: 11, a chimeric intron exemplified by SEQ ID NO: 19, optionally a cloning site exemplified by SEQ ID NO: 30, a GJB2 coding region exemplified by SEQ ID NO: 1, optionally a cloning sequence exemplified by SEQ ID NO: 33, a 3′ UTR exemplified by SEQ ID NO: 22, optionally a cloning site exemplified by SEQ ID NO: 34, a poly(A) site exemplified by SEQ ID NO: 25, optionally a cloning site exemplified by SEQ ID NO: 32, and a 3′ ITR exemplified by SEQ ID NO: 9.

In some embodiments, an exemplary rAAVAnc80 particle comprises a construct represented by SEQ ID NO: 47.

In one embodiment, an exemplary construct comprises: a 5′ ITR exemplified by SEQ ID NO: 8, optionally a cloning site exemplified by SEQ ID NO: 29, a promoter/enhancer region as exemplified by SEQ ID NO: 17, optionally a cloning site exemplified by SEQ ID NO: 35, a GJB2 coding region exemplified by SEQ ID NO: 1, optionally a cloning site exemplified by SEQ ID NO: 31, a filler sequence exemplified by SEQ ID NO: 27, optionally a cloning site exemplified by SEQ ID NO: 36, a poly(A) site exemplified by SEQ ID NO: 25, optionally a cloning site exemplified by SEQ ID NO: 37, and a 3′ ITR exemplified by SEQ ID NO: 9.

In some embodiments, an exemplary rAAVAnc80 particle comprises a construct represented by SEQ ID NO: 48.

In one embodiment, an exemplary construct comprises: a 5′ ITR exemplified by SEQ ID NO: 8, optionally a cloning site exemplified by SEQ ID NO: 29, a promoter/enhancer region as exemplified by SEQ ID NO: 17, optionally a cloning site exemplified by SEQ ID NO: 35, a GJB2 coding region exemplified by SEQ ID NO: 1, optionally a cloning site exemplified by SEQ ID NO: 31, a filler sequence exemplified by SEQ ID NO: 28, optionally a cloning site exemplified by SEQ ID NO: 36, a poly(A) site exemplified by SEQ ID NO: 25, optionally a cloning site exemplified by SEQ ID NO: 37, and a 3′ ITR exemplified by SEQ ID NO: 9.

In some embodiments, an exemplary rAAVAnc80 particle comprises a construct represented by SEQ ID NO: 49.

In one embodiment, an exemplary construct comprises: a 5′ ITR exemplified by SEQ ID NO: 8, optionally a cloning site exemplified by SEQ ID NO: 29, a promoter/enhancer region as exemplified by SEQ ID NO: 17, optionally a cloning site exemplified by SEQ ID NO: 35, a GJB2 coding region exemplified by SEQ ID NO: 1, optionally a cloning site exemplified by SEQ ID NO: 31, a poly(A) site exemplified by SEQ ID NO: 25, optionally a cloning site exemplified by SEQ ID NO: 32, and a 3′ ITR exemplified by SEQ ID NO: 9.

In some embodiments, an exemplary rAAVAnc80 particle comprises a construct represented by SEQ ID NO: 50.

In one embodiment, an exemplary construct comprises: a 5′ ITR exemplified by SEQ ID NO: 8, optionally a cloning site exemplified by SEQ ID NO: 29, a CMV enhancer exemplified by SEQ ID NO: 18, a CBA promoter exemplified by SEQ ID NO: 10, a chimeric intron exemplified by SEQ ID NO: 19, optionally a cloning site exemplified by SEQ ID NO: 30, a GJB2 coding region exemplified by SEQ ID NO: 1, optionally a cloning site exemplified by SEQ ID NO: 31, a poly(A) site exemplified by SEQ ID NO: 25, optionally a cloning site exemplified by SEQ ID NO: 32, and a 3′ ITR exemplified by SEQ ID NO: 9.

In some embodiments, an exemplary rAAVAnc80 particle comprises a construct represented by SEQ ID NO: 51.

In one embodiment, an exemplary construct comprises: a 5′ ITR exemplified by SEQ ID NO: 8, optionally a cloning site exemplified by SEQ ID NO: 29, a CMV enhancer exemplified by SEQ ID NO: 18, a CBA promoter exemplified by SEQ ID NO: 10, a chimeric intron exemplified by SEQ ID NO: 19, optionally a cloning site exemplified by SEQ ID NO: 30, a GJB2 coding region exemplified by SEQ ID NO: 1, optionally a cloning site exemplified by SEQ ID NO: 33, a 3′ UTR exemplified by SEQ ID NO: 22, optionally a cloning site exemplified by SEQ ID NO: 34, a poly(A) site exemplified by SEQ ID NO: 25, optionally a cloning site exemplified by SEQ ID NO: 32, and a 3′ ITR exemplified by SEQ ID NO: 9.

In some embodiments, an exemplary rAAVAnc80 particle comprises a construct represented by SEQ ID NO: 82.

In one embodiment, an exemplary construct comprises: a 5′ ITR exemplified by SEQ ID NO: 52, optionally a cloning site exemplified by SEQ ID NO: 70, a CAG enhancer/promoter exemplified by SEQ ID NO: 14, optionally a cloning site exemplified by SEQ ID NO: 72, a GJB2 5′UTR sequence exemplified by SEQ ID NO: 66, optionally a cloning site exemplified by SEQ ID NO: 73, a GJB2 coding region exemplified by SEQ ID NO: 1, a linker sequence exemplified by SEQ ID NO: 77, a FLAG sequence with stop codon exemplified by SEQ ID NO: 81, a 3′ UTR exemplified by SEQ ID NO: 67, optionally a cloning site exemplified by SEQ ID NO: 75, a poly(A) site exemplified by SEQ ID NO: 25, optionally a cloning site exemplified by SEQ ID NO: 76, and a 3′ ITR exemplified by SEQ ID NO: 53.

In some embodiments, an exemplary rAAVAnc80 particle comprises a construct represented by SEQ ID NO: 83.

In one embodiment, an exemplary construct comprises: a 5′ ITR exemplified by SEQ ID NO: 52, optionally a cloning site exemplified by SEQ ID NO: 70, a CMV/CBA enhancer/promoter exemplified by SEQ ID NO: 12, a chimeric intron exemplified by SEQ ID NO: 64, optionally a cloning site exemplified by SEQ ID NO: 72, a GJB2 5′UTR sequence exemplified by SEQ ID NO: 66, optionally a cloning site exemplified by SEQ ID NO: 73, a GJB2 coding region exemplified by SEQ ID NO: 1, a linker sequence exemplified by SEQ ID NO: 77, a FLAG sequence with stop codon exemplified by SEQ ID NO: 81, a 3′ UTR exemplified by SEQ ID NO: 67, optionally a cloning site exemplified by SEQ ID NO: 75, a poly(A) site exemplified by SEQ ID NO: 25, optionally a cloning site exemplified by SEQ ID NO: 76, and a 3′ ITR exemplified by SEQ ID NO: 53.

In some embodiments, an exemplary rAAVAnc80 particle comprises a construct represented by SEQ ID NO: 84.

In one embodiment, an exemplary construct comprises: a 5′ ITR exemplified by SEQ ID NO: 52, optionally a cloning site exemplified by SEQ ID NO: 70, a CMV enhancer exemplified by SEQ ID NO: 63, a human GJB2 promoter exemplified by SEQ ID NO: 61, optionally a cloning site exemplified by SEQ ID NO: 72, a GJB2 5′UTR sequence exemplified by SEQ ID NO: 66, optionally a cloning site exemplified by SEQ ID NO: 73, a GJB2 coding region exemplified by SEQ ID NO: 1, a linker sequence exemplified by SEQ ID NO: 77, a FLAG sequence with stop codon exemplified by SEQ ID NO: 81, a 3′ UTR exemplified by SEQ ID NO: 67, optionally a cloning site exemplified by SEQ ID NO: 75, a poly(A) site exemplified by SEQ ID NO: 25, optionally a cloning site exemplified by SEQ ID NO: 76, and a 3′ ITR exemplified by SEQ ID NO: 53.

In some embodiments, an exemplary rAAVAnc80 particle comprises a construct represented by SEQ ID NO: 85.

In one embodiment, an exemplary construct comprises: a 5′ ITR exemplified by SEQ ID NO: 52, optionally a cloning site exemplified by SEQ ID NO: 70, a CMV enhancer exemplified by SEQ ID NO: 63, a GFAP enhancer-promoter exemplified by SEQ ID NO: 62, optionally a cloning site exemplified by SEQ ID NO: 72, a GJB2 5′UTR sequence exemplified by SEQ ID NO: 66, optionally a cloning site exemplified by SEQ ID NO: 73, a GJB2 coding region exemplified by SEQ ID NO: 1, a linker sequence exemplified by SEQ ID NO: 77, a FLAG sequence with stop codon exemplified by SEQ ID NO: 81, a 3′ UTR exemplified by SEQ ID NO: 67, optionally a cloning site exemplified by SEQ ID NO: 75, a poly(A) site exemplified by SEQ ID NO: 25, optionally a cloning site exemplified by SEQ ID NO: 76, and a 3′ ITR exemplified by SEQ ID NO: 53.

In some embodiments, an exemplary rAAVAnc80 particle comprises a construct represented by SEQ ID NO: 86.

In one embodiment, an exemplary construct comprises: a 5′ ITR exemplified by SEQ ID NO: 52, optionally a cloning site exemplified by SEQ ID NO: 71, a human GFAP enhancer-promoter exemplified by SEQ ID NO: 62, optionally a cloning site exemplified by SEQ ID NO: 72, a GJB2 5′UTR sequence exemplified by SEQ ID NO: 66, optionally a cloning site exemplified by SEQ ID NO: 73, a GJB2 coding region exemplified by SEQ ID NO: 1, a linker sequence exemplified by SEQ ID NO: 77, a FLAG sequence with stop codon exemplified by SEQ ID NO: 81, a 3′ UTR exemplified by SEQ ID NO: 67, optionally a cloning site exemplified by SEQ ID NO: 75, a poly(A) site exemplified by SEQ ID NO: 25, optionally a cloning site exemplified by SEQ ID NO: 76, and a 3′ ITR exemplified by SEQ ID NO: 53.

In some embodiments, an exemplary rAAVAnc80 particle comprises a construct represented by SEQ ID NO: 87.

In one embodiment, an exemplary construct comprises: a 5′ ITR exemplified by SEQ ID NO: 52, optionally a cloning site exemplified by SEQ ID NO: 70, a human GFAP enhancer-promoter exemplified by SEQ ID NO: 62, optionally a cloning site exemplified by SEQ ID NO: 72, a GJB2 5′UTR sequence exemplified by SEQ ID NO: 66, optionally a cloning site exemplified by SEQ ID NO: 73, a GJB2 coding region exemplified by SEQ ID NO: 1, a linker sequence exemplified by SEQ ID NO: 77, a FLAG sequence with stop codon exemplified by SEQ ID NO: 81, a destabilization domain exemplified by SEQ ID NO: 80, a 3′ UTR exemplified by SEQ ID NO: 68, optionally a cloning site exemplified by SEQ ID NO: 34, a poly(A) site exemplified by SEQ ID NO: 25, optionally a cloning site exemplified by SEQ ID NO: 76, and a 3′ ITR exemplified by SEQ ID NO: 53.

In some embodiments, an exemplary rAAVAnc80 particle comprises a construct represented by SEQ ID NO: 88.

In one embodiment, an exemplary construct comprises: a 5′ ITR exemplified by SEQ ID NO: 52, optionally a cloning site exemplified by SEQ ID NO: 70, a GJB2 enhancer region exemplified by SEQ ID NO: 65, a GJB2 promoter exemplified by SEQ ID NO: 61, optionally a cloning site exemplified by SEQ ID NO: 72, a GJB2 5′UTR sequence exemplified by SEQ ID NO: 20, optionally a cloning site exemplified by SEQ ID NO: 74, a GJB2 coding region exemplified by SEQ ID NO: 1, a linker sequence exemplified by SEQ ID NO: 77, a FLAG sequence with stop codon exemplified by SEQ ID NO: 81, a 3′ UTR exemplified by SEQ ID NO: 67, optionally a cloning site exemplified by SEQ ID NO: 76, and a 3′ ITR exemplified by SEQ ID NO: 53.

In some aspects, the rAAVAnc80 particle comprises a construct comprising the nucleic acid sequence of SEQ ID NO: 94.

In one aspect, the construct comprises a 5′ ITR comprising the nucleic acid sequence of SEQ ID NO: 52, optionally a cloning site comprising the nucleic acid sequence of SEQ ID NO: 71, a GDF6 promoter sequence comprising the nucleic acid sequence of SEQ ID NO: 90; a hGJB2 minimal promoter comprising the nucleic acid sequence of SEQ ID NO: 91, optionally a cloning site comprising the nucleic acid sequence of SEQ ID NO: 92; optionally a synthetic barcode comprising the nucleic acid sequence of SEQ ID NO: 93; a 5′UTR sequence comprising the nucleic acid sequence of SEQ ID NO: 66, a GJB2 coding region comprising the nucleic acid sequence of SEQ ID NO: 1, a linker sequence exemplified by SEQ ID NO: 77, a FLAG sequence with stop codon comprising the nucleic acid sequence of SEQ ID NO: 81, a 3′ UTR comprising the nucleic acid sequence of SEQ ID NO: 67, a poly(A) comprising the nucleic acid sequence of SEQ ID NO: 25, optionally a cloning site comprising the nucleic acid sequence of SEQ ID NO: 76, and a 3′ ITR comprising the nucleic acid sequence of SEQ ID NO: 53.

In some aspects, the rAAVAnc80 particle comprises a construct comprising the nucleic acid sequence of SEQ ID NO: 97.

In one aspect, the construct comprises a 5′ ITR comprising the nucleic acid sequence of SEQ ID NO: 52, optionally a cloning site comprising the nucleic acid sequence of SEQ ID NO: 71, a IGFBP2 promoter sequence comprising the nucleic acid sequence of SEQ ID NO: 95; a hGJB2 minimal promoter comprising the nucleic acid sequence of SEQ ID NO: 91, optionally a cloning site comprising the nucleic acid sequence of SEQ ID NO: 92; optionally a synthetic barcode comprising the nucleic acid sequence of SEQ ID NO: 96; a 5′UTR sequence comprising the nucleic acid sequence of SEQ ID NO: 66, a GJB2 coding region comprising the nucleic acid sequence of SEQ ID NO: 1, a linker sequence exemplified by SEQ ID NO: 77, a FLAG sequence with stop codon comprising the nucleic acid sequence of SEQ ID NO: 81, a 3′ UTR comprising the nucleic acid sequence of SEQ ID NO: 67, a poly(A) comprising the nucleic acid sequence of SEQ ID NO: 25, optionally a cloning site comprising the nucleic acid sequence of SEQ ID NO: 76, and a 3′ ITR comprising the nucleic acid sequence of SEQ ID NO: 53.

In some aspects, the rAAVAnc80 particle comprises a construct comprising the nucleic acid sequence of SEQ ID NO: 100.

In one aspect, the construct comprises a 5′ ITR comprising the nucleic acid sequence of SEQ ID NO: 52, optionally a cloning site comprising the nucleic acid sequence of SEQ ID NO: 71, a RBP7 promoter sequence comprising the nucleic acid sequence of SEQ ID NO: 98; a hGJB2 minimal promoter comprising the nucleic acid sequence of SEQ ID NO: 91, optionally a cloning site comprising the nucleic acid sequence of SEQ ID NO: 92; optionally a synthetic barcode comprising the nucleic acid sequence of SEQ ID NO: 99; a 5′UTR sequence comprising the nucleic acid sequence of SEQ ID NO: 66, a GJB2 coding region comprising the nucleic acid sequence of SEQ ID NO: 1, a linker sequence exemplified by SEQ ID NO: 77, a FLAG sequence with stop codon comprising the nucleic acid sequence of SEQ ID NO: 81, a 3′ UTR comprising the nucleic acid sequence of SEQ ID NO: 67, a poly(A) comprising the nucleic acid sequence of SEQ ID NO: 25, optionally a cloning site comprising the nucleic acid sequence of SEQ ID NO: 76, and a 3′ ITR comprising the nucleic acid sequence of SEQ ID NO: 53.

In some aspects, the rAAVAnc80 particle comprises a construct comprising the nucleic acid sequence of SEQ ID NO: 103.

In one aspect, the construct comprises a 5′ ITR comprising the nucleic acid sequence of SEQ ID NO: 52, optionally a cloning site comprising the nucleic acid sequence of SEQ ID NO: 71, a GJB6 promoter sequence comprising the nucleic acid sequence of SEQ ID NO: 101; a hGJB2 minimal promoter comprising the nucleic acid sequence of SEQ ID NO: 91, optionally a cloning site comprising the nucleic acid sequence of SEQ ID NO: 92; optionally a synthetic barcode comprising the nucleic acid sequence of SEQ ID NO: 102; a 5′UTR sequence comprising the nucleic acid sequence of SEQ ID NO: 66, a GJB2 coding region comprising the nucleic acid sequence of SEQ ID NO: 1, a linker sequence exemplified by SEQ ID NO: 77, a FLAG sequence with stop codon comprising the nucleic acid sequence of SEQ ID NO: 81, a 3′ UTR comprising the nucleic acid sequence of SEQ ID NO: 67, a poly(A) comprising the nucleic acid sequence of SEQ ID NO: 25, optionally a cloning site comprising the nucleic acid sequence of SEQ ID NO: 76, and a 3′ ITR comprising the nucleic acid sequence of SEQ ID NO: 53.

In some aspects, the rAAVAnc80 particle comprises a construct comprising the nucleic acid sequence of SEQ ID NO: 106.

In one aspect, the construct comprises a 5′ ITR comprising the nucleic acid sequence of SEQ ID NO: 52, optionally a cloning site comprising the nucleic acid sequence of SEQ ID NO: 71, a PARM1 promoter sequence comprising the nucleic acid sequence of SEQ ID NO: 104; a hGJB2 minimal promoter comprising the nucleic acid sequence of SEQ ID NO: 91, optionally a cloning site comprising the nucleic acid sequence of SEQ ID NO: 92; optionally a synthetic barcode comprising the nucleic acid sequence of SEQ ID NO: 105; a 5′UTR sequence comprising the nucleic acid sequence of SEQ ID NO: 66, a GJB2 coding region comprising the nucleic acid sequence of SEQ ID NO: 1, a linker sequence exemplified by SEQ ID NO: 77, a FLAG sequence with stop codon comprising the nucleic acid sequence of SEQ ID NO: 81, a 3′ UTR comprising the nucleic acid sequence of SEQ ID NO: 67, a poly(A) comprising the nucleic acid sequence of SEQ ID NO: 25, optionally a cloning site comprising the nucleic acid sequence of SEQ ID NO: 76, and a 3′ ITR comprising the nucleic acid sequence of SEQ ID NO: 53.

Exemplary Construct sequence
(SEQ ID NO: 45)
TTGGCCACTCCCTCTCTGCGCGCTCGCTCGCTCACTGAGGCCGCCCGGGCAAAGCCCGGGCGTCG
GGCGACCTTTGGTCGCCCGGCCTCAGTGAGCGAGCGAGCGCGCAGAGAGGGAGTGGCCAACTCCA
TCACTAGGGGTTCCTTTGTCGACGCGGCCGCACGCGTGACATTGATTATTGACTAGTTATTAATA
GTAATCAATTACGGGGTCATTAGTTCATAGCCCATATATGGAGTTCCGCGTTACATAACTTACGG
TAAATGGCCCGCCTGGCTGACCGCCCAACGACCCCCGCCCATTGACGTCAATAATGACGTATGTT
CCCATAGTAACGCCAATAGGGACTTTCCATTGACGTCAATGGGTGGACTATTTACGGTAAACTGC
CCACTTGGCAGTACATCAAGTGTATCATATGCCAAGTACGCCCCCTATTGACGTCAATGACGGTA
AATGGCCCGCCTGGCATTATGCCCAGTACATGACCTTATGGGACTTTCCTACTTGGCAGTACATC
TACGTATTAGTCATCGCTATTACCATGGGTCGAGGTGAGCCCCACGTTCTGCTTCACTCTCCCCA
TCTCCCCCCCCTCCCCACCCCCAATTTTGTATTTATTTATTTTTTAATTATTTTGTGCAGCGATG
GGGGCGGGGGGGGGGGGGGGGCGCGCGCCAGGCGGGGCGGGGCGGGGCGAGGGGCGGGGCGGGGC
GAGGCGGAGAGGTGCGGCGGCAGCCAATCAGAGCGGCGCGCTCCGAAAGTTTCCTTTTATGGCGA
GGCGGCGGCGGCGGCGGCCCTATAAAAAGCGAAGCGCGCGGCGGGCGGGAGTCGCTGCGTTGCCT
TCGCCCCGTGCCCCGCTCCGCGCCGCCTCGCGCCGCCCGCCCCGGCTCTGACTGACCGCGTTACT
CCCACAGGTGAGCGGGCGGGACGGCCCTTCTCCTCCGGGCTGTAATTAGCGCTTGGTTTAATGAC
GGCTCGTTTCTTTTCTGTGGCTGCGTGAAAGCCTTAAAGGGCTCCGGGAGGGCCCTTTGTGCGGG
GGGGAGCGGCTCGGGGGGTGCGTGCGTGTGTGTGTGCGTGGGGAGCGCCGCGTGCGGCCCGCGCT
GCCCGGCGGCTGTGAGCGCTGCGGGCGCGGCGCGGGGCTTTGTGCGCTCCGCGTGTGCGCGAGGG
GAGCGCGGCCGGGGGCGGTGCCCCGCGGTGCGGGGGGGCTGCGAGGGGAACAAAGGCTGCGTGCG
GGGTGTGTGCGTGGGGGGGTGAGCAGGGGGTGTGGGCGCGGCGGTCGGGCTGTAACCCCCCCCTG
CACCCCCCTCCCCGAGTTGCTGAGCACGGCCCGGCTTCGGGTGCGGGGCTCCGTGCGGGGCGTGG
CGCGGGGCTCGCCGTGCCGGGCGGGGGGTGGCGGCAGGTGGGGGTGCCGGGCGGGGCGGGGCCGC
CTCGGGCCGGGGAGGGCTCGGGGGAGGGGCGCGGCGGCCCCCGGAGCGCCGGCGGCTGTCGAGGC
GCGGCGAGCCGCAGCCATTGCCTTTTATGGTAATCGTGCGAGAGGGCGCAGGGACTTCCTTTGTC
CCAAATCTGTGCGGAGCCGAAATCTGGGAGGCGCCGCCGcACCCCCTCTAGCGGGCGCGGGGCGA
AGCGGTGCGGCGCCGGCAGGAAGGAAATGGGCGGGGAGGGCCTTCGTGCGTCGCCGCGCCGCCGT
CCCCTTCTCCCTCTCCAGCCTCGGGGCTGTCCGCGGGGGGACGGCTGCCTTCGGGGGGGACGGGG
CAGGGCGGGGTTCGGCTTCTGGCGTGTGACCGGCGGCTCTAGAGCCTCTGCTAACCATGTTCATG
CCTTCTTCTTTTTCCTACAGCTCCTGGGCAACGTGCTGGTTATTGTGACCGGTGCCACCATGGAT
TGGGGCACGCTGCAGACGATCCTGGGGGGTGTGAACAAACACTCCACCAGCATTGGAAAGATCTG
GCTCACCGTCCTCTTCATTTTTCGCATTATGATCCTCGTTGTGGCTGCAAAGGAGGTGTGGGGAG
ATGAGCAGGCCGACTTTGTCTGCAACACCCTGCAGCCAGGCTGCAAGAACGTGTGCTACGATCAC
TACTTCCCCATCTCCCACATCCGGCTATGGGCCCTGCAGCTGATCTTCGTGTCCACGCCAGCGCT
CCTAGTGGCCATGCACGTGGCCTACCGGAGACATGAGAAGAAGAGGAAGTTCATCAAGGGGGAGA
TAAAGAGTGAATTTAAGGACATCGAGGAGATCAAAACCCAGAAGGTCCGCATCGAAGGCTCCCTG
TGGTGGACCTACACAAGCAGCATCTTCTTCCGGGTCATCTTCGAAGCCGCCTTCATGTACGTCTT
CTATGTCATGTACGACGGCTTCTCCATGCAGCGGCTGGTGAAGTGCAACGCCTGGCCTTGTCCCA
ACACTGTGGACTGCTTTGTGTCCCGGCCCACGGAGAAGACTGTCTTCACAGTGTTCATGATTGCA
GTGTCTGGAATTTGCATCCTGCTGAATGTCACTGAATTGTGTTATTTGCTAATTAGATATTGTTC
TGGGAAGTCAAAAAAGCCAGTTTAAGAGCTCGCTGATCAGCCTCGACTGTGCCTTCTAGTTGCCA
GCCATCTGTTGTTTGCCCCTCCCCCGTGCCTTCCTTGACCCTGGAAGGTGCCACTCCCACTGTCC
TTTCCTAATAAAATGAGGAAATTGCATCGCATTGTCTGAGTAGGTGTCATTCTATTCTGGGGGGT
GGGGTGGGGCAGGACAGCAAGGGGGAGGATTGGGAAGACAATAGCAGGCATGCTGGGGATGCGGT
GGGCTCTATGGAAGCTTGAATTCAGCTGACGTGCCTCGGACCGCCTAGGAGGAACCCCTAGTGAT
GGAGTTGGCCACTCCCTCTCTGCGCGCTCGCTCGCTCACTGAGGCCGGGCGACCAAAGGTCGCCC
GACGCCCGGGCTTTGCCCGGGCGGCCTCAGTGAGCGAGCGAGCGCGCAGAGAGGGAGTGGCCAA
Exemplary Construct sequence
(SEQ ID NO: 46)
TTGGCCACTCCCTCTCTGCGCGCTCGCTCGCTCACTGAGGCCGCCCGGGCAAAGCCCGGGCGTCG
GGCGACCTTTGGTCGCCCGGCCTCAGTGAGCGAGCGAGCGCGCAGAGAGGGAGTGGCCAACTCCA
TCACTAGGGGTTCCTTTGTCGACGCGGCCGCACGCGTGACATTGATTATTGACTAGTTATTAATA
GTAATCAATTACGGGGTCATTAGTTCATAGCCCATATATGGAGTTCCGCGTTACATAACTTACGG
TAAATGGCCCGCCTGGCTGACCGCCCAACGACCCCCGCCCATTGACGTCAATAATGACGTATGTT
CCCATAGTAACGCCAATAGGGACTTTCCATTGACGTCAATGGGTGGACTATTTACGGTAAACTGC
CCACTTGGCAGTACATCAAGTGTATCATATGCCAAGTACGCCCCCTATTGACGTCAATGACGGTA
AATGGCCCGCCTGGCATTATGCCCAGTACATGACCTTATGGGACTTTCCTACTTGGCAGTACATC
TACGTATTAGTCATCGCTATTACCATGGGTCGAGGTGAGCCCCACGTTCTGCTTCACTCTCCCCA
TCTCCCCCCCCTCCCCACCCCCAATTTTGTATTTATTTATTTTTTAATTATTTTGTGCAGCGATG
GGGGCGGGGGGGGGGGGGGGGCGCGCGCCAGGCGGGGCGGGGCGGGGCGAGGGGCGGGGCGGGGC
GAGGCGGAGAGGTGCGGCGGCAGCCAATCAGAGCGGCGCGCTCCGAAAGTTTCCTTTTATGGCGA
GGCGGCGGCGGCGGCGGCCCTATAAAAAGCGAAGCGCGCGGCGGGCGGGAGTCGCTGCGTTGCCT
TCGCCCCGTGCCCCGCTCCGCGCCGCCTCGCGCCGCCCGCCCCGGCTCTGACTGACCGCGTTACT
CCCACAGGTGAGCGGGCGGGACGGCCCTTCTCCTCCGGGCTGTAATTAGCGCTTGGTTTAATGAC
GGCTCGTTTCTTTTCTGTGGCTGCGTGAAAGCCTTAAAGGGCTCCGGGAGGGCCCTTTGTGCGGG
GGGGAGCGGCTCGGGGGGTGCGTGCGTGTGTGTGTGCGTGGGGAGCGCCGCGTGCGGCCCGCGCT
GCCCGGCGGCTGTGAGCGCTGCGGGCGCGGCGCGGGGCTTTGTGCGCTCCGCGTGTGCGCGAGGG
GAGCGCGGCCGGGGGCGGTGCCCCGCGGTGCGGGGGGGCTGCGAGGGGAACAAAGGCTGCGTGCG
GGGTGTGTGCGTGGGGGGGTGAGCAGGGGGTGTGGGCGCGGCGGTCGGGCTGTAACCCCCCCCTG
CACCCCCCTCCCCGAGTTGCTGAGCACGGCCCGGCTTCGGGTGCGGGGCTCCGTGCGGGGCGTGG
CGCGGGGCTCGCCGTGCCGGGCGGGGGGTGGCGGCAGGTGGGGGTGCCGGGCGGGGCGGGGCCGC
CTCGGGCCGGGGAGGGCTCGGGGGAGGGGCGCGGCGGCCCCCGGAGCGCCGGCGGCTGTCGAGGC
GCGGCGAGCCGCAGCCATTGCCTTTTATGGTAATCGTGCGAGAGGGCGCAGGGACTTCCTTTGTC
CCAAATCTGTGCGGAGCCGAAATCTGGGAGGCGCCGCCGCACCCCCTCTAGCGGGCGCGGGGCGA
AGCGGTGCGGCGCCGGCAGGAAGGAAATGGGCGGGGAGGGCCTTCGTGCGTCGCCGCGCCGCCGT
CCCCTTCTCCCTCTCCAGCCTCGGGGCTGTCCGCGGGGGGACGGCTGCCTTCGGGGGGGACGGGG
CAGGGCGGGGTTCGGCTTCTGGCGTGTGACCGGCGGCTCTAGAGCCTCTGCTAACCATGTTCATG
CCTTCTTCTTTTTCCTACAGCTCCTGGGCAACGTGCTGGTTATTGTGACCGGTGCCACCATGGAT
TGGGGCACGCTGCAGACGATCCTGGGGGGTGTGAACAAACACTCCACCAGCATTGGAAAGATCTG
GCTCACCGTCCTCTTCATTTTTCGCATTATGATCCTCGTTGTGGCTGCAAAGGAGGTGTGGGGAG
ATGAGCAGGCCGACTTTGTCTGCAACACCCTGCAGCCAGGCTGCAAGAACGTGTGCTACGATCAC
TACTTCCCCATCTCCCACATCCGGCTATGGGCCCTGCAGCTGATCTTCGTGTCCACGCCAGCGCT
CCTAGTGGCCATGCACGTGGCCTACCGGAGACATGAGAAGAAGAGGAAGTTCATCAAGGGGGAGA
TAAAGAGTGAATTTAAGGACATCGAGGAGATCAAAACCCAGAAGGTCCGCATCGAAGGCTCCCTG
TGGTGGACCTACACAAGCAGCATCTTCTTCCGGGTCATCTTCGAAGCCGCCTTCATGTACGTCTT
CTATGTCATGTACGACGGCTTCTCCATGCAGCGGCTGGTGAAGTGCAACGCCTGGCCTTGTCCCA
ACACTGTGGACTGCTTTGTGTCCCGGCCCACGGAGAAGACTGTCTTCACAGTGTTCATGATTGCA
GTGTCTGGAATTTGCATCCTGCTGAATGTCACTGAATTGTGTTATTTGCTAATTAGATATTGTTC
TGGGAAGTCAAAAAAGCCAGTTTAAGAGCTCCGCATTGCCCAGTTGTTAGATTAAGAAATAGACA
GCATGAGAGGGATGAGGCAACCCGTGCTCAGCTGTCAAGGCTCAGTCGCTAGCATTTCCCAACAC
AAAGATTCTGACCTTAAATGCAACCATTTGAAACCCCTGTAGGCCTCAGGTGAAACTCCAGATGC
CACAATGGAGCCTCTGCTCCCCTAAAGCCTCAAAACAAAGGCCTAATTCTATGCCTGTCTTAATT
TTCTTTCACTTAAGTTAGTTCCACTGAGACCCCAGGCTGTTAGGGGTTATTGGTGTAAGGTACTT
TCATATTTTAAACAGAGGATATCGGCATTTGTTTCTTTCTCTGAGGACAAGAGAAAAAAGCCAGG
TTCCACAGAGGACACAGAGAAGGTTTGGGTGTCCTCCTGGGGTTCTTTTTGCCAACTTTCCCCAC
GTTAAAGGTGAACATTGGTTCTTTCATTTGCTTTGGAAGTTTTAATCTCTAACAGTGGACAAAGT
TACCAGTGCCTTAAACTCTGTTACACTTTTTGGAAGTGAAAACTTTGTAGTATGATAGGTTATTT
TGATGTAAAGATGTTCTGGATACCATTATATGTTCCCCCTGTTTCAGAGGCTCAGATTGTAATAT
GTAAATGGTATGTCATTCGCTACTATGATTTAATTTGAAATATGGTCTTTTGGTTATGAATACTT
TGCAGCACAGCTGAGAGGCTGTCTGTTGTATTCATTGTGGTCATAGCACCTAACAACATTGTAGC
CTCAATCGAGTGAGACAGACTAGAAGTTCCTAGTGATGGCTTATGATAGCAAATGGCCTCATGTC
AAATATTTAGATGTAATTTTGTGTAAGAAATACAGACTGGATGTACCACCAACTACTACCTGTAA
TGACAGGCCTGTCCAACACATCTCCCTTTTCCATGACTGTGGTAGCCAGCATCGGAAAGAACGCT
GATTTAAAGAGGTCGCTTGGGAATTTTATTGACACAGTACCATTTAATGGGGAGGACAAAATGGG
GCAGGGGAGGGAGAAGTTTCTGTCGTTAAAAACAGATTTGGAAAGACTGGACTCTAAAGTCTGTT
GATTAAAGATGAGCTTTGTCTACTTCAAAAGTTTGTTTGCTTACCCCTTCAGCCTCCAATTTTTT
AAGTGAAAATATAGCTAATAACATGTGAAAAGAATAGAAGCTAAGGTTTAGATAAATATTGAGCA
GATCTATAGGAAGATTGAACCTGAATATTGCCATTATGCTTGACATGGTTTCCAAAAAATGGTAC
TCCACATATTTCAGTGAGGGTAAGTATTTTCCTGTTGTCAAGAATAGCATTGTAAAAGCATTTTG
TAATAATAAAGAATAGCTTTAATGATATGCTTGTAACTAAAATAATTTTGTAATGTATCAAATAC
ATTTAAAACATTAAAATATAATCTCTATAATAAGCTGATCAGCCTCGACTGTGCCTTCTAGTTGC
CAGCCATCTGTTGTTTGCCCCTCCCCCGTGCCTTCCTTGACCCTGGAAGGTGCCACTCCCACTGT
CCTTTCCTAATAAAATGAGGAAATTGCATCGCATTGTCTGAGTAGGTGTCATTCTATTCTGGGGG
GTGGGGTGGGGCAGGACAGCAAGGGGGAGGATTGGGAAGACAATAGCAGGCATGCTGGGGATGCG
GTGGGCTCTATGGAAGCTTGAATTCAGCTGACGTGCCTCGGACCGCCTAGGAGGAACCCCTAGTG
ATGGAGTTGGCCACTCCCTCTCTGCGCGCTCGCTCGCTCACTGAGGCCGGGCGACCAAAGGTCGC
CCGACGCCCGGGCTTTGCCCGGGCGGCCTCAGTGAGCGAGCGAGCGCGCAGAGAGGGAGTGGCCA
A
Exemplary Construct sequence
(SEQ ID NO: 47)
TTGGCCACTCCCTCTCTGCGCGCTCGCTCGCTCACTGAGGCCGCCCGGGCAAAGCCCGGGCGTCG
GGCGACCTTTGGTCGCCCGGCCTCAGTGAGCGAGCGAGCGCGCAGAGAGGGAGTGGCCAACTCCA
TCACTAGGGGTTCCTTTGTCGACGCGGCCGCACGCGTAAGCTTCGGTGAATTTAAAACGTTTGGT
GGCAGTGGGTCAAGTAGCCAGGCGGCTGCGCTAGAGTACCCCGAAGGGACATCGGCGACACCACA
AACCTCGCGCTGGCGGCTCGCCCGCGCcTTTTTCCCCTCCCGCGCGCGCCCGGCCCCACTCGCAC
CCCGGGCGGTGCCATCGCGTCCACTTCCCCGGCCGCCCCATTCCAGCTCCGGAGCTCGGCCGCAG
AAACGCCCGCTCCAGAAGGCGGCCCCCGCCCCCCGGCCCAAGGACGTGTGTTGGTCCAGCCCCCC
GGTTCCCCGAGACCCACGCGGCCGGGCAACCGCTCTGGGTCTCGCGGTCCCTCCCCGCGCCAGGT
TCCTGGCCGGGCAGTCCGGGGCCGGCGGGCTCACCTGCGTCGGGAGGAAGCGCGGCGGGGCCGGG
GCGGGGGTCTCGGCGTTGGGGTCTCTGCGCTGGGGCTCCTGCGCTCCTAGGCGGGTCCTGGGCCG
GGCGCCGCCGAGGGGCTCCGAGTCGGGGAGAGGAGCGCGCGGGCGCTGCGGGGCCGCAACACCTG
TCTCCCGCCGTGGCGCCTTTTAACCGCACCCCACACCCCGCCTCTTCCCTCGGAGACTGGGAAAG
TTACGGAGGGGGCGGCGCCGCGGGCGGAGCGCGCCCGGCCTCTGGGTCCTCAGAGCTTCCCGGGT
CCGCGAACCCCCGACCGCCCCCGAAAGCCCCGAACCCCCCAAGTCCCCTTCGAGGTCCCGATCTC
CTAGTTCCTTTGAGCCCCCATGAGTTCCCCAAGTGCCCCCAGCGCCCTGAGTCTCCCCCGGTTAC
CCCGAGCGCCGCCTCCCCCAGCCCCTTGGCGGCCCGGGTGAAGCGGGGGCGGCTGAGAGTCGGGA
CCCCCCAGGAAGCGGCGCCCCAGACCCCGGCTCCGGCGCTGTGCCGTGGGCGGGGTTCAGGGATG
GCTGTGGTCGTTGTCCTCTGTACTCCGCATAGTGCGAGAGGACTTGGCATTTATGAGCGCTTCTT
TAATTTTTTATTGTTAGAGAAACAGGCATTCCTCCAAGGACTGAAGATCTGTTCGAGTCGCGGAG
GCTGCGCGGGCCCGCGAGGCTCTCGCAGGGGGACCTAGGCTGGGTGGCGGGGCAGTGCCCTCTGG
AATGGGGGTTAACGGTGGCCGAGGAGGGGGCGCCGCTGGTGCCGGCGAAGTCCCCGCTTCTTTCT
CCCCTCAAAATCTCACCAATCCGAACGAACGCCTTCTCGAATTTCCGATTTTATTCAATTACTTT
CAACAATGTGCCAAGGACTAAGGTTGGGGGCGGTGGGAGAGACAAGCCTCGTTTTTGCCATGGCC
GGCAGGGGGGTCCCGCCATCTGCGGAGGGTGCCCCCCGCGGCCCCCGGCCCAGCCAACTTCCTCC
TCTTTTCGCAACTGGGGAACTGCAAGGAGGTGACTCCTTTCGGGGTGAGGAGGCCCAGACTTTTC
AGAAAGGAAAGAGGGCAGGTAAAACCTGCCAAGCCCCTTCCTGCTCGATGCACACAGCACGAAAG
GGGGAAACTGATAGGATTCTGCGGAAGCTTGGCATTCCGGTACTGTTGGTAAAGCCACCAGCAAA
CCGCCCAGAGTAGAAGACCGGTGGCCACCATGGATTGGGGCACGCTGCAGACGATCCTGGGGGGT
GTGAACAAACACTCCACCAGCATTGGAAAGATCTGGCTCACCGTCCTCTTCATTTTTCGCATTAT
GATCCTCGTTGTGGCTGCAAAGGAGGTGTGGGGAGATGAGCAGGCCGACTTTGTCTGCAACACCC
TGCAGCCAGGCTGCAAGAACGTGTGCTACGATCACTACTTCCCCATCTCCCACATCCGGCTATGG
GCCCTGCAGCTGATCTTCGTGTCCACGCCAGCGCTCCTAGTGGCCATGCACGTGGCCTACCGGAG
ACATGAGAAGAAGAGGAAGTTCATCAAGGGGGAGATAAAGAGTGAATTTAAGGACATCGAGGAGA
TCAAAACCCAGAAGGTCCGCATCGAAGGCTCCCTGTGGTGGACCTACACAAGCAGCATCTTCTTC
CGGGTCATCTTCGAAGCCGCCTTCATGTACGTCTTCTATGTCATGTACGACGGCTTCTCCATGCA
GCGGCTGGTGAAGTGCAACGCCTGGCCTTGTCCCAACACTGTGGACTGCTTTGTGTCCCGGCCCA
CGGAGAAGACTGTCTTCACAGTGTTCATGATTGCAGTGTCTGGAATTTGCATCCTGCTGAATGTC
ACTGAATTGTGTTATTTGCTAATTAGATATTGTTCTGGGAAGTCAAAAAAGCCAGTTTAAGAGCT
CGCTGATCAGCCTCGACTGTGCCTTCTAGTTGCCAGCCATCTGTTGTTTGCCCCTCCCCCGTGCC
TTCCTTGACCCTGGAAGGTGCCACTCCCACTGTCCTTTCCTAATAAAATGAGGAAATTGCATCGC
ATTGTCTGAGTAGGTGTCATTCTATTCTGGGGGGTGGGGTGGGGCAGGACAGCAAGGGGGAGGAT
TGGGAAGACAATAGCAGGCATGCTGGGGATGCGGTGGGCTCTATGGAAGCTTGAATTCCTTCTTC
TGGAGTCTTTTCTGGAATAATTCTGGGAGTGGGCTCAGCCTGCGGGAGAGTAACATTTTTATAAC
TTGATAGATGTAGCTGAGATGCCTCCCAGAGGGGAGACCCGCCTCTCCTCCGGCAGCTGTGCACG
TAGGCTTGTTCCCAGCAGCCTGGCCAGGGTGGTCCACCTGGTGTTTCTCATCTTCTTTCCCCGGA
GCGCTGACTCCTGCGCGTCCTCTTGGAAGACTCTTGACAGGACGGGTGTTTTATGGGTGTGATTC
AGTGTCCTCTTGCATCAGTTCAATGTGGTGGTGTTCAATCAACCCTTGTAGCGTTAGCAAAATTT
GCTCAAGTCATTCCGCAGGAATGTCTGTGTCTTGCTTCCAAGAAAGCTTGTAAGTGCCGGCAACA
GGCCAAGCAGCTCACAAACCTGACCACAAGCCTGTGAGTAATTGTGGGGCAGCACTTAGCAGTCT
TTTATTTTCGACTTATTAAAGTCTCATCTTGGCCTCACCTTCTCCCTGGAAGGTGGCGTGGGTGG
GAACCACTGGGTCAGATCTTTTTCACCCTTGCCGTGGAGCCAGTTTCCTGTTGCATGTGGGGGAA
GCAACATGTGGTGAAGAGTATAGAAAACGAAAACATGTGGGTACAGTATGTATAAGTGGAGGGAA
CAAACTCATAATTCCAACTAGTTTCTCATGAGAGACTCATGAATCATTGTGGTAGTTCTCAATAT
AAACTTAATCTAGGCCGGATGTGGTGGCTCACACCTGTAATCTCAGCACTCTGGGTGGATCACTT
GAGGTCAGGAGTTTGAGACCAGTCTGACCAACATGGAGAAACCCCATCGCTACTAAAAATACAAA
ATTATCCAGATGTGGTGGCTCACACCTGTAATCCCAGCACTTTGGGAGGCTGAGGCGGGTGGATC
ACTTGAGGTCAGGAGTTTGAGACCAGCCTGACCAACATGGAGAAACTGTGTCTCTACTAAAAATA
CAAAATTAGCTGGGCGTGGTGACGCATGCCTGTAATCCCAGCTATTTGGAGGCCGAAGCAGGAGC
TGACGTGCCTCGGACCGCCTAGGAGGAACCCCTAGTGATGGAGTTGGCCACTCCCTCTCTGCGCG
CTCGCTCGCTCACTGAGGCCGGGCGACCAAAGGTCGCCCGACGCCCGGGCTTTGCCCGGGCGGCC
TCAGTGAGCGAGCGAGCGCGCAGAGAGGGAGTGGCCAA
Exemplary Construct sequence
(SEQ ID NO: 48)
TTGGCCACTCCCTCTCTGCGCGCTCGCTCGCTCACTGAGGCCGCCCGGGCAAAGCCCGGGCGTCG
GGCGACCTTTGGTCGCCCGGCCTCAGTGAGCGAGCGAGCGCGCAGAGAGGGAGTGGCCAACTCCA
TCACTAGGGGTTCCTTTGTCGACGCGGCCGCACGCGTAAGCTTCGGTGAATTTAAAACGTTTGGT
GGCAGTGGGTCAAGTAGCCAGGCGGCTGCGCTAGAGTACCCCGAAGGGACATCGGCGACACCACA
AACCTCGCGCTGGCGGCTCGCCCGCGCCTTTTTCCCCTCCCGCGCGCGCCCGGCCCCACTCGCAC
CCCGGGCGGTGCCATCGCGTCCACTTCCCCGGCCGCCCCATTCCAGCTCCGGAGCTCGGCCGCAG
AAACGCCCGCTCCAGAAGGCGGCCCCCGCCCCCCGGCCCAAGGACGTGTGTTGGTCCAGCCCCCC
GGTTCCCCGAGACCCACGCGGCCGGGcAACCGCTCTGGGTCTCGCGGTCCCTCCCCGCGCCAGGT
TCCTGGCCGGGCAGTCCGGGGCCGGCGGGCTCACCTGCGTCGGGAGGAAGCGCGGCGGGGCCGGG
GCGGGGGTCTCGGCGTTGGGGTCTCTGCGCTGGGGCTCCTGCGCTCCTAGGCGGGTCCTGGGCCG
GGCGCCGCCGAGGGGCTCCGAGTCGGGGAGAGGAGCGCGCGGGCGCTGCGGGGCCGCAACACCTG
TCTCCCGCCGTGGCGCCTTTTAACCGCACCCCACACCCCGCCTCTTCCCTCGGAGACTGGGAAAG
TTACGGAGGGGGCGGCGCCGCGGGCGGAGCGCGCCCGGCCTCTGGGTCCTCAGAGCTTCCCGGGT
CCGCGAACCCCCGACCGCCCCCGAAAGCCCCGAACCCCCCAAGTCCCCTTCGAGGTCCCGATCTC
CTAGTTCCTTTGAGCCCCCATGAGTTCCCCAAGTGCCCCCAGCGCCCTGAGTCTCCCCCGGTTAC
CCCGAGCGCCGCCTCCCCCAGCCCCTTGGCGGCCCGGGTGAAGCGGGGGCGGCTGAGAGTCGGGA
CCCCCCAGGAAGCGGCGCCCCAGACCCCGGCTCCGGCGCTGTGCCGTGGGCGGGGTTCAGGGATG
GCTGTGGTCGTTGTCCTCTGTACTCCGCATAGTGCGAGAGGACTTGGCATTTATGAGCGCTTCTT
TAATTTTTTATTGTTAGAGAAACAGGCATTCCTCCAAGGACTGAAGATCTGTTCGAGTCGCGGAG
GCTGCGCGGGCCCGCGAGGCTCTCGCAGGGGGACCTAGGCTGGGTGGCGGGGCAGTGCCCTCTGG
AATGGGGGTTAACGGTGGCCGAGGAGGGGGCGCCGCTGGTGCCGGCGAAGTCCCCGCTTCTTTCT
CCCCTCAAAATCTCACCAATCCGAACGAACGCCTTCTCGAATTTCCGATTTTATTCAATTACTTT
CAACAATGTGCCAAGGACTAAGGTTGGGGGCGGTGGGAGAGACAAGCCTCGTTTTTGCCATGGCC
GGCAGGGGGGTCCCGCCATCTGCGGAGGGTGCCCCCCGCGGCCCCCGGCCCAGCCAACTTCCTCC
TCTTTTCGCAACTGGGGAACTGCAAGGAGGTGACTCCTTTCGGGGTGAGGAGGCCCAGACTTTTC
AGAAAGGAAAGAGGGCAGGTAAAACCTGCCAAGCCCCTTCCTGCTCGATGCACACAGCACGAAAG
GGGGAAACTGATAGGATTCTGCGGAAGCTTGGCATTCCGGTACTGTTGGTAAAGCCACCAGCAAA
CCGCCCAGAGTAGAAGACCGGTGGCCACCATGGATTGGGGCACGCTGCAGACGATCCTGGGGGGT
GTGAACAAACACTCCACCAGCATTGGAAAGATCTGGCTCACCGTCCTCTTCATTTTTCGCATTAT
GATCCTCGTTGTGGCTGCAAAGGAGGTGTGGGGAGATGAGCAGGCCGACTTTGTCTGCAACACCC
TGCAGCCAGGCTGCAAGAACGTGTGCTACGATCACTACTTCCCCATCTCCCACATCCGGCTATGG
GCCCTGCAGCTGATCTTCGTGTCCACGCCAGCGCTCCTAGTGGCCATGCACGTGGCCTACCGGAG
ACATGAGAAGAAGAGGAAGTTCATCAAGGGGGAGATAAAGAGTGAATTTAAGGACATCGAGGAGA
TCAAAACCCAGAAGGTCCGCATCGAAGGCTCCCTGTGGTGGACCTACACAAGCAGCATCTTCTTC
CGGGTCATCTTCGAAGCCGCCTTCATGTACGTCTTCTATGTCATGTACGACGGCTTCTCCATGCA
GCGGCTGGTGAAGTGCAACGCCTGGCCTTGTCCCAACACTGTGGACTGCTTTGTGTCCCGGCCCA
CGGAGAAGACTGTCTTCACAGTGTTCATGATTGCAGTGTCTGGAATTTGCATCCTGCTGAATGTC
ACTGAATTGTGTTATTTGCTAATTAGATATTGTTCTGGGAAGTCAAAAAAGCCAGTTTAAGAGCT
CGCTGATCAGCCTCGACTGTGCCTTCTAGTTGCCAGCCATCTGTTGTTTGCCCCTCCCCCGTGCC
TTCCTTGACCCTGGAAGGTGCCACTCCCACTGTCCTTTCCTAATAAAATGAGGAAATTGCATCGC
ATTGTCTGAGTAGGTGTCATTCTATTCTGGGGGGTGGGGTGGGGCAGGACAGCAAGGGGGAGGAT
TGGGAAGACAATAGCAGGCATGCTGGGGATGCGGTGGGCTCTATGGAAGCTTGAATTCCCTGTAA
AGCCAATTCCAACCCACTTGTAATTAAGAGAAAATCCCACGGTTCCTAATTGAAAGTCCTTTGTT
CTATTTCTTGGGTATTTGTGTTTTAGGCCTTATTTTTAGATGCATCATTAAAGATTTTTAAAGTC
CTTTCAGGCATCAGGACTGATGATGCTGAATGATGGAGGGTTGTGGATAAGTTTTTTTGTTTTTT
TTTTAACCAGGTTAAAGGCTTTCCTGTTATCCTACTATGCTTAATTAAGAGCTGTATTTCTTAAT
ATCATTGGTGCCTGATTAGATTTAACTTTTAGATACAGTCTGTAAGATTTTTGAACCAGAAAAAC
CTAAATAACTTATGACTGTTAGCAGTCATATTCTAGAAGAAGCAAATGTACTGAATTCTTATGTA
CCTAGGATTTTAAGGGAGTACATACAAATCTTTCCTCAGTAGCAGGTACTTTATTTTTATAACAC
ACACATTTAAGCTGAGTTAAATATGCAGAACTGGTTGTACTTCTTTGGCAGGAAAAGGGAAGCTT
AGGATATCTTGTGACCAACTACCTCTTCCTTCTCAAATAACTGGCAAATAACTTCAGGAAAATCC
AGTTATGTTGTGTCATATTGCACCCCCTAGGAAGTACTGGATTCTTAGTCTTGAGTGACTTTTAA
ATAAAGCTACCTTTTTCTCTTTCTTACATCGCAAGATCTTCAAATGTACCATTCCCGCACAGAGA
GTCCAAGGTAAAAGGACTGAAACCAAACTTTGTTTTTGTAAGTATTTTGGTCAGTGCAATGAGTT
CAGAGACCAGGAGGTTAATGATTGTGAAGTCTTGTCAACAGCAACACCGTGTATGACCTGTGGTG
CTTAGATGTTCAGAAACCCCAAGGTTAAAATGTCCCTGACCACATATCAGGCAAAAGGAATGTAA
GGAAAACCAACTTAATCCTTTTGTCAAGAAGTATAAATGATGTATCTTTCCAATCGGGTTGCATT
GACTTTTGGGTCCAAATAGCTTGTGTCCACAGGCATCTTCAGCTGACGTGCCTCGGACCGCCTAG
GAGGAACCCCTAGTGATGGAGTTGGCCACTCCCTCTCTGCGCGCTCGCTCGCTCACTGAGGCCGG
GCGACCAAAGGTCGCCCGACGCCCGGGCTTTGCCCGGGCGGCCTCAGTGAGCGAGCGAGCGCGCA
GAGAGGGAGTGGCCAA
Exemplary Construct sequence
(SEQ ID NO: 49)
TTGGCCACTCCCTCTCTGCGCGCTCGCTCGCTCACTGAGGCCGCCCGGGCAAAGCCCGGGCGTCG
GGCGACCTTTGGTCGCCCGGCCTCAGTGAGCGAGCGAGCGCGCAGAGAGGGAGTGGCCAACTCCA
TCACTAGGGGTTCCTTTGTCGACGCGGCCGCACGCGTAAGCTTCGGTGAATTTAAAACGTTTGGT
GGCAGTGGGTCAAGTAGCCAGGCGGCTGCGCTAGAGTACCCCGAAGGGACATCGGCGACACCACA
AACCTCGCGCTGGCGGCTCGCCCGCGCCTTTTTCCCCTCCCGCGCGCGCCCGGCCCCACTCGCAC
CCCGGGCGGTGCCATCGCGTCCACTTCCCCGGCCGCCCCATTCCAGCTCCGGAGCTCGGCCGCAG
AAACGCCCGCTCCAGAAGGCGGCCCCCGCCCCCCGGCCCAAGGACGTGTGTTGGTCCAGCCCCCC
GGTTCCCCGAGACCCACGCGGCCGGGCAACCGCTCTGGGTCTCGCGGTCCCTCCCCGCGCCAGGT
TCCTGGCCGGGCAGTCCGGGGCCGGCGGGCTCACCTGCGTCGGGAGGAAGCGCGGCGGGGCCGGG
GCGGGGGTCTCGGCGTTGGGGTCTCTGCGCTGGGGCTCCTGCGCTCCTAGGCGGGTCCTGGGCCG
GGCGCCGCCGAGGGGCTCCGAGTCGGGGAGAGGAGCGCGCGGGCGCTGCGGGGCCGCAACACCTG
TCTCCCGCCGTGGCGCCTTTTAACCGCACCCCACACCCCGCCTCTTCCCTCGGAGACTGGGAAAG
TTACGGAGGGGGCGGCGCCGCGGGCGGAGCGCGCCCGGCCTCTGGGTCCTCAGAGCTTCCCGGGT
CCGCGAACCCCCGACCGCCCCCGAAAGCCCCGAACCCCCCAAGTCCCCTTCGAGGTCCCGATCTC
CTAGTTCCTTTGAGCCCCCATGAGTTCCCCAAGTGCCCCCAGCGCCCTGAGTCTCCCCCGGTTAC
CCCGAGCGCCGCCTCCCCCAGCCCCTTGGCGGCCCGGGTGAAGCGGGGGCGGCTGAGAGTCGGGA
CCCCCCAGGAAGCGGCGCCCCAGACCCCGGCTCCGGCGCTGTGCCGTGGGCGGGGTTCAGGGATG
GCTGTGGTCGTTGTCCTCTGTACTCCGCATAGTGCGAGAGGACTTGGCATTTATGAGCGCTTCTT
TAATTTTTTATTGTTAGAGAAACAGGCATTCCTCCAAGGACTGAAGATCTGTTCGAGTCGCGGAG
GCTGCGCGGGCCCGCGAGGCTCTCGCAGGGGGACCTAGGCTGGGTGGCGGGGCAGTGCCCTCTGG
AATGGGGGTTAACGGTGGCCGAGGAGGGGGCGCCGCTGGTGCCGGCGAAGTCCCCGCTTCTTTCT
CCCCTCAAAATCTCACCAATCCGAACGAACGCCTTCTCGAATTTCCGATTTTATTCAATTACTTT
CAACAATGTGCCAAGGACTAAGGTTGGGGGCGGTGGGAGAGACAAGCCTCGTTTTTGCCATGGCC
GGCAGGGGGGTCCCGCCATCTGCGGAGGGTGCCCCCCGCGGCCCCCGGCCCAGCCAACTTCCTCC
TCTTTTCGCAACTGGGGAACTGCAAGGAGGTGACTCCTTTCGGGGTGAGGAGGCCCAGACTTTTC
AGAAAGGAAAGAGGGCAGGTAAAACCTGCCAAGCCCCTTCCTGCTCGATGCACACAGCACGAAAG
GGGGAAACTGATAGGATTCTGCGGAAGCTTGGCATTCCGGTACTGTTGGTAAAGCCACCAGCAAA
CCGCCCAGAGTAGAAGACCGGTGGCCACCATGGATTGGGGCACGCTGCAGACGATCCTGGGGGGT
GTGAACAAACACTCCACCAGCATTGGAAAGATCTGGCTCACCGTCCTCTTCATTTTTCGCATTAT
GATCCTCGTTGTGGCTGCAAAGGAGGTGTGGGGAGATGAGCAGGCCGACTTTGTCTGCAACACCC
TGCAGCCAGGCTGCAAGAACGTGTGCTACGATCACTACTTCCCCATCTCCCACATCCGGCTATGG
GCCCTGCAGCTGATCTTCGTGTCCACGCCAGCGCTCCTAGTGGCCATGCACGTGGCCTACCGGAG
ACATGAGAAGAAGAGGAAGTTCATCAAGGGGGAGATAAAGAGTGAATTTAAGGACATCGAGGAGA
TCAAAACCCAGAAGGTCCGCATCGAAGGCTCCCTGTGGTGGACCTACACAAGCAGCATCTTCTTC
CGGGTCATCTTCGAAGCCGCCTTCATGTACGTCTTCTATGTCATGTACGACGGCTTCTCCATGCA
GCGGCTGGTGAAGTGCAACGCCTGGCCTTGTCCCAACACTGTGGACTGCTTTGTGTCCCGGCCCA
CGGAGAAGACTGTCTTCACAGTGTTCATGATTGCAGTGTCTGGAATTTGCATCCTGCTGAATGTC
ACTGAATTGTGTTATTTGCTAATTAGATATTGTTCTGGGAAGTCAAAAAAGCCAGTTTAAGAGCT
CGCTGATCAGCCTCGACTGTGCCTTCTAGTTGCCAGCCATCTGTTGTTTGCCCCTCCCCCGTGCC
TTCCTTGACCCTGGAAGGTGCCACTCCCACTGTCCTTTCCTAATAAAATGAGGAAATTGCATCGC
ATTGTCTGAGTAGGTGTCATTCTATTCTGGGGGGTGGGGTGGGGCAGGACAGCAAGGGGGAGGAT
TGGGAAGACAATAGCAGGCATGCTGGGGATGCGGTGGGCTCTATGGAAGCTTGAATTCAGCTGAC
GTGCCTCGGACCGCCTAGGAGGAACCCCTAGTGATGGAGTTGGCCACTCCCTCTCTGCGCGCTCG
CTCGCTCACTGAGGCCGGGCGACCAAAGGTCGCCCGACGCCCGGGCTTTGCCCGGGCGGCCTCAG
TGAGCGAGCGAGCGCGCAGAGAGGGAGTGGCCAA
Exemplary Construct sequence
(SEQ ID NO: 50)
TTGGCCACTCCCTCTCTGCGCGCTCGCTCGCTCACTGAGGCCGCCCGGGCAAAGCCCGGGCGTCG
GGCGACCTTTGGTCGCCCGGCCTCAGTGAGCGAGCGAGCGCGCAGAGAGGGAGTGGCCAACTCCA
TCACTAGGGGTTCCTTTGTCGACGCGGCCGCACGCGTGACATTGATTATTGACTAGTTATTAATA
GTAATCAATTACGGGGTCATTAGTTCATAGCCCATATATGGAGTTCCGCGTTACATAACTTACGG
TAAATGGCCCGCCTGGCTGACCGCCCAACGACCCCCGCCCATTGACGTCAATAATGACGTATGTT
CCCATAGTAACGCCAATAGGGACTTTCCATTGACGTCAATGGGTGGACTATTTACGGTAAACTGC
CCACTTGGCAGTACATCAAGTGTATCATATGCCAAGTACGCCCCCTATTGACGTCAATGACGGTA
AATGGCCCGCCTGGCATTATGCCCAGTACATGACCTTATGGGACTTTCCTACTTGGCAGTACATC
TACGTATTAGTCATCGCTATTACCATGGGTCGAGGTGAGCCCCACGTTCTGCTTCACTCTCCCCA
TCTCCCCCCCCTCCCCACCCCCAATTTTGTATTTATTTATTTTTTAATTATTTTGTGCAGCGATG
GGGGCGGGGGGGGGGGGGGCGCGCGCCAGGCGGGGCGGGGCGGGGCGAGGGGCGGGGCGGGGCGA
GGCGGAGAGGTGCGGCGGCAGCCAATCAGAGCGGCGCGCTCCGAAAGTTTCCTTTTATGGCGAGG
CGGCGGCGGCGGCGGCCCTATAAAAAGCGAAGCGCGCGGCGGGCGGGAGTCGCTGCGTTGCCTTC
GCCCCGTGCCCCGCTCCGCGCCGCCTCGCGCCGCCCGCCCCGGCTCTGACTGACCGCGTTACTCC
CACAGGTGAGCGGGCGGGACGGCCCTTCTCCTCCGGGCTGTAATTAGCGCTTGGTTTAATGACGG
CTCGTTTCTTTTCTGTGGCTGCGTGAAAGCCTTAAAGGGCTCCGGGAGGGCCCTTTGTGCGGGGG
GGAGCGGCTCGGGGGGTGCGTGCGTGTGTGTGTGCGTGGGGAGCGCCGCGTGCGGCCCGCGCTGC
CCGGCGGCTGTGAGCGCTGCGGGCGCGGCGCGGGGCTTTGTGCGCTCCGCGTGTGCGCGAGGGGA
GCGCGGCCGGGGGCGGTGCCCCGCGGTGCGGGGGGGCTGCGAGGGGAACAAAGGCTGCGTGCGGG
GTGTGTGCGTGGGGGGGTGAGCAGGGGGTGTGGGCGCGGCGGTCGGGCTGTAACCCCCCCCTGCA
CCCCCCTCCCCGAGTTGCTGAGCACGGCCCGGCTTCGGGTGCGGGGCTCCGTGCGGGGCGTGGCG
CGGGGCTCGCCGTGCCGGGCGGGGGGTGGCGGCAGGTGGGGGTGCCGGGCGGGGCGGGGCCGCCT
CGGGCCGGGGAGGGCTCGGGGGAGGGGCGCGGCGGCCCCCGGAGCGCCGGCGGCTGTCGAGGCGC
GGCGAGCCGCAGCCATTGCCTTTTATGGTAATCGTGCGAGAGGGCGCAGGGACTTCCTTTGTCCC
AAATCTGTGCGGAGCCGAAATCTGGGAGGCGCCGCCGCACCCCCTCTAGCGGGCGCGGGGCGAAG
CGGTGCGGCGCCGGCAGGAAGGAAATGGGCGGGGAGGGCCTTCGTGCGTCGCCGCGCCGCCGTCC
CCTTCTCCCTCTCCAGCCTCGGGGCTGTCCGCGGGGGGACGGCTGCCTTCGGGGGGGACGGGGCA
GGGCGGGGTTCGGCTTCTGGCGTGTGACCGGCGGCTCTAGAGCCTCTGCTAACCATGTTCATGCC
TTCTTCTTTTTCCTACAGCTCCTGGGCAACGTGCTGGTTATTGTGACCGGTGCCACCATGGATTG
GGGCACGCTGCAGACGATCCTGGGGGGTGTGAACAAACACTCCACCAGCATTGGAAAGATCTGGC
TCACCGTCCTCTTCATTTTTCGCATTATGATCCTCGTTGTGGCTGCAAAGGAGGTGTGGGGAGAT
GAGCAGGCCGACTTTGTCTGCAACACCCTGCAGCCAGGCTGCAAGAACGTGTGCTACGATCACTA
CTTCCCCATCTCCCACATCCGGCTATGGGCCCTGCAGCTGATCTTCGTGTCCACGCCAGCGCTCC
TAGTGGCCATGCACGTGGCCTACCGGAGACATGAGAAGAAGAGGAAGTTCATCAAGGGGGAGATA
AAGAGTGAATTTAAGGACATCGAGGAGATCAAAACCCAGAAGGTCCGCATCGAAGGCTCCCTGTG
GTGGACCTACACAAGCAGCATCTTCTTCCGGGTCATCTTCGAAGCCGCCTTCATGTACGTCTTCT
ATGTCATGTACGACGGCTTCTCCATGCAGCGGCTGGTGAAGTGCAACGCCTGGCCTTGTCCCAAC
ACTGTGGACTGCTTTGTGTCCCGGCCCACGGAGAAGACTGTCTTCACAGTGTTCATGATTGCAGT
GTCTGGAATTTGCATCCTGCTGAATGTCACTGAATTGTGTTATTTGCTAATTAGATATTGTTCTG
GGAAGTCAAAAAAGCCAGTTTAAGAGCTCGCTGATCAGCCTCGACTGTGCCTTCTAGTTGCCAGC
CATCTGTTGTTTGCCCCTCCCCCGTGCCTTCCTTGACCCTGGAAGGTGCCACTCCCACTGTCCTT
TCCTAATAAAATGAGGAAATTGCATCGCATTGTCTGAGTAGGTGTCATTCTATTCTGGGGGGTGG
GGTGGGGCAGGACAGCAAGGGGGAGGATTGGGAAGACAATAGCAGGCATGCTGGGGATGCGGTGG
GCTCTATGGAAGCTTGAATTCAGCTGACGTGCCTCGGACCGCCTAGGAGGAACCCCTAGTGATGG
AGTTGGCCACTCCCTCTCTGCGCGCTCGCTCGCTCACTGAGGCCGGGCGACCAAAGGTCGCCCGA
CGCCCGGGCTTTGCCCGGGCGGCCTCAGTGAGCGAGCGAGCGCGCAGAGAGGGAGTGGCCAA
Exemplary Construct sequence
(SEQ ID NO: 51)
TTGGCCACTCCCTCTCTGCGCGCTCGCTCGCTCACTGAGGCCGCCCGGGCAAAGCCCGGGCGTCG
GGCGACCTTTGGTCGCCCGGCCTCAGTGAGCGAGCGAGCGCGCAGAGAGGGAGTGGCCAACTCCA
TCACTAGGGGTTCCTTTGTCGACGCGGCCGCACGCGTGACATTGATTATTGACTAGTTATTAATA
GTAATCAATTACGGGGTCATTAGTTCATAGCCCATATATGGAGTTCCGCGTTACATAACTTACGG
TAAATGGCCCGCCTGGCTGACCGCCCAACGACCCCCGCCCATTGACGTCAATAATGACGTATGTT
CCCATAGTAACGCCAATAGGGACTTTCCATTGACGTCAATGGGTGGACTATTTACGGTAAACTGC
CCACTTGGCAGTACATCAAGTGTATCATATGCCAAGTACGCCCCCTATTGACGTCAATGACGGTA
AATGGCCCGCCTGGCATTATGCCCAGTACATGACCTTATGGGACTTTCCTACTTGGCAGTACATC
TACGTATTAGTCATCGCTATTACCATGGGTCGAGGTGAGCCCCACGTTCTGCTTCACTCTCCCCA
TCTCCCCCCCCTCCCCACCCCCAATTTTGTATTTATTTATTTTTTAATTATTTTGTGCAGCGATG
GGGGCGGGGGGGGGGGGGGCGCGCGCCAGGCGGGGCGGGGCGGGGCGAGGGGCGGGGCGGGGCGA
GGCGGAGAGGTGCGGCGGCAGCCAATCAGAGCGGCGCGCTCCGAAAGTTTCCTTTTATGGCGAGG
CGGCGGCGGCGGCGGCCCTATAAAAAGCGAAGCGCGCGGCGGGCGGGAGTCGCTGCGTTGCCTTC
GCCCCGTGCCCCGCTCCGCGCCGCCTCGCGCCGCCCGCCCCGGCTCTGACTGACCGCGTTACTCC
CACAGGTGAGCGGGCGGGACGGCCCTTCTCCTCCGGGCTGTAATTAGCGCTTGGTTTAATGACGG
CTCGTTTCTTTTCTGTGGCTGCGTGAAAGCCTTAAAGGGCTCCGGGAGGGCCCTTTGTGCGGGGG
GGAGCGGCTCGGGGGGTGCGTGCGTGTGTGTGTGCGTGGGGAGCGCCGCGTGCGGCCCGCGCTGC
CCGGCGGCTGTGAGCGCTGCGGGCGCGGCGCGGGGCTTTGTGCGCTCCGCGTGTGCGCGAGGGGA
GCGCGGCCGGGGGCGGTGCCCCGCGGTGCGGGGGGGCTGCGAGGGGAACAAAGGCTGCGTGCGGG
GTGTGTGCGTGGGGGGGTGAGCAGGGGGTGTGGGCGCGGCGGTCGGGCTGTAACCCCCCCCTGCA
CCCCCCTCCCCGAGTTGCTGAGCACGGCCCGGCTTCGGGTGCGGGGCTCCGTGCGGGGCGTGGCG
CGGGGCTCGCCGTGCCGGGCGGGGGGTGGCGGCAGGTGGGGGTGCCGGGCGGGGCGGGGCCGCCT
CGGGCCGGGGAGGGCTCGGGGGAGGGGCGCGGCGGCCCCCGGAGCGCCGGCGGCTGTCGAGGCGC
GGCGAGCCGCAGCCATTGCCTTTTATGGTAATCGTGCGAGAGGGCGCAGGGACTTCCTTTGTCCC
AAATCTGTGCGGAGCCGAAATCTGGGAGGCGCCGCCGCACCCCCTCTAGCGGGCGCGGGGCGAAG
CGGTGCGGCGCCGGCAGGAAGGAAATGGGCGGGGAGGGCCTTCGTGCGTCGCCGCGCCGCCGTCC
CCTTCTCCCTCTCCAGCCTCGGGGCTGTCCGCGGGGGGACGGCTGCCTTCGGGGGGGACGGGGCA
GGGCGGGGTTCGGCTTCTGGCGTGTGACCGGCGGCTCTAGAGCCTCTGCTAACCATGTTCATGCC
TTCTTCTTTTTCCTACAGCTCCTGGGCAACGTGCTGGTTATTGTGACCGGTGCCACCATGGATTG
GGGCACGCTGCAGACGATCCTGGGGGGTGTGAACAAACACTCCACCAGCATTGGAAAGATCTGGC
TCACCGTCCTCTTCATTTTTCGCATTATGATCCTCGTTGTGGCTGCAAAGGAGGTGTGGGGAGAT
GAGCAGGCCGACTTTGTCTGCAACACCCTGCAGCCAGGCTGCAAGAACGTGTGCTACGATCACTA
CTTCCCCATCTCCCACATCCGGCTATGGGCCCTGCAGCTGATCTTCGTGTCCACGCCAGCGCTCC
TAGTGGCCATGCACGTGGCCTACCGGAGACATGAGAAGAAGAGGAAGTTCATCAAGGGGGAGATA
AAGAGTGAATTTAAGGACATCGAGGAGATCAAAACCCAGAAGGTCCGCATCGAAGGCTCCCTGTG
GTGGACCTACACAAGCAGCATCTTCTTCCGGGTCATCTTCGAAGCCGCCTTCATGTACGTCTTCT
ATGTCATGTACGACGGCTTCTCCATGCAGCGGCTGGTGAAGTGCAACGCCTGGCCTTGTCCCAAC
ACTGTGGACTGCTTTGTGTCCCGGCCCACGGAGAAGACTGTCTTCACAGTGTTCATGATTGCAGT
GTCTGGAATTTGCATCCTGCTGAATGTCACTGAATTGTGTTATTTGCTAATTAGATATTGTTCTG
GGAAGTCAAAAAAGCCAGTTTAAGAGCTCCGCATTGCCCAGTTGTTAGATTAAGAAATAGACAGC
ATGAGAGGGATGAGGCAACCCGTGCTCAGCTGTCAAGGCTCAGTCGCTAGCATTTCCCAACACAA
AGATTCTGACCTTAAATGCAACCATTTGAAACCCCTGTAGGCCTCAGGTGAAACTCCAGATGCCA
CAATGGAGCCTCTGCTCCCCTAAAGCCTCAAAACAAAGGCCTAATTCTATGCCTGTCTTAATTTT
CTTTCACTTAAGTTAGTTCCACTGAGACCCCAGGCTGTTAGGGGTTATTGGTGTAAGGTACTTTC
ATATTTTAAACAGAGGATATCGGCATTTGTTTCTTTCTCTGAGGACAAGAGAAAAAAGCCAGGTT
CCACAGAGGACACAGAGAAGGTTTGGGTGTCCTCCTGGGGTTCTTTTTGCCAACTTTCCCCACGT
TAAAGGTGAACATTGGTTCTTTCATTTGCTTTGGAAGTTTTAATCTCTAACAGTGGACAAAGTTA
CCAGTGCCTTAAACTCTGTTACACTTTTTGGAAGTGAAAACTTTGTAGTATGATAGGTTATTTTG
ATGTAAAGATGTTCTGGATACCATTATATGTTCCCCCTGTTTCAGAGGCTCAGATTGTAATATGT
AAATGGTATGTCATTCGCTACTATGATTTAATTTGAAATATGGTCTTTTGGTTATGAATACTTTG
CAGCACAGCTGAGAGGCTGTCTGTTGTATTCATTGTGGTCATAGCACCTAACAACATTGTAGCCT
CAATCGAGTGAGACAGACTAGAAGTTCCTAGTGATGGCTTATGATAGCAAATGGCCTCATGTCAA
ATATTTAGATGTAATTTTGTGTAAGAAATACAGACTGGATGTACCACCAACTACTACCTGTAATG
ACAGGCCTGTCCAACACATCTCCCTTTTCCATGACTGTGGTAGCCAGCATCGGAAAGAACGCTGA
TTTAAAGAGGTCGCTTGGGAATTTTATTGACACAGTACCATTTAATGGGGAGGACAAAATGGGGC
AGGGGAGGGAGAAGTTTCTGTCGTTAAAAACAGATTTGGAAAGACTGGACTCTAAAGTCTGTTGA
TTAAAGATGAGCTTTGTCTACTTCAAAAGTTTGTTTGCTTACCCCTTCAGCCTCCAATTTTTTAA
GTGAAAATATAGCTAATAACATGTGAAAAGAATAGAAGCTAAGGTTTAGATAAATATTGAGCAGA
TCTATAGGAAGATTGAACCTGAATATTGCCATTATGCTTGACATGGTTTCCAAAAAATGGTACTC
CACATATTTCAGTGAGGGTAAGTATTTTCCTGTTGTCAAGAATAGCATTGTAAAAGCATTTTGTA
ATAATAAAGAATAGCTTTAATGATATGCTTGTAACTAAAATAATTTTGTAATGTATCAAATACAT
TTAAAACATTAAAATATAATCTCTATAATAAGCTGATCAGCCTCGACTGTGCCTTCTAGTTGCCA
GCCATCTGTTGTTTGCCCCTCCCCCGTGCCTTCCTTGACCCTGGAAGGTGCCACTCCCACTGTCC
TTTCCTAATAAAATGAGGAAATTGCATCGCATTGTCTGAGTAGGTGTCATTCTATTCTGGGGGGT
GGGGTGGGGCAGGACAGCAAGGGGGAGGATTGGGAAGACAATAGCAGGCATGCTGGGGATGCGGT
GGGCTCTATGGAAGCTTGAATTCAGCTGACGTGCCTCGGACCGCCTAGGAGGAACCCCTAGTGAT
GGAGTTGGCCACTCCCTCTCTGCGCGCTCGCTCGCTCACTGAGGCCGGGCGACCAAAGGTCGCCC
GACGCCCGGGCTTTGCCCGGGCGGCCTCAGTGAGCGAGCGAGCGCGCAGAGAGGGAGTGGCCAA
Exemplary Construct sequence
(SEQ ID NO: 82)
CTGCGCGCTCGCTCGCTCACTGAGGCCGCCCGGGCGTCGGGCGACCTTTGGTCGCCCGGCCTCAG
TGAGCGAGCGAGCGCGCAGAGAGGGAGTGGCCAACTCCATCACTAGGGGTTCCTGCGGCCGCACG
CGTGACATTGATTATTGACTAGTTATTAATAGTAATCAATTACGGGGTCATTAGTTCATAGCCCA
TATATGGAGTTCCGCGTTACATAACTTACGGTAAATGGCCCGCCTGGCTGACCGCCCAACGACCC
CCGCCCATTGACGTCAATAATGACGTATGTTCCCATAGTAACGCCAATAGGGACTTTCCATTGAC
GTCAATGGGTGGACTATTTACGGTAAACTGCCCACTTGGCAGTACATCAAGTGTATCATATGCCA
AGTACGCCCCCTATTGACGTCAATGACGGTAAATGGCCCGCCTGGCATTATGCCCAGTACATGAC
CTTATGGGACTTTCCTACTTGGCAGTACATCTACGTATTAGTCATCGCTATTACCATGGGTCGAG
GTGAGCCCCACGTTCTGCTTCACTCTCCCCATCTCCCCCCCCTCCCCACCCCCAATTTTGTATTT
ATTTATTTTTTAATTATTTTGTGCAGCGATGGGGGCGGGGGGGGGGGGGGCGCGCGCCAGGCGGG
GCGGGGCGGGGCGAGGGGCGGGGCGGGGCGAGGCGGAGAGGTGCGGCGGCAGCCAATCAGAGCGG
CGCGCTCCGAAAGTTTCCTTTTATGGCGAGGCGGCGGCGGCGGCGGCCCTATAAAAAGCGAAGCG
CGCGGCGGGCGGGAGTCGCTGCGTTGCCTTCGCCCCGTGCCCCGCTCCGCGCCGCCTCGCGCCGC
CCGCCCCGGCTCTGACTGACCGCGTTACTCCCACAGGTGAGCGGGCGGGACGGCCCTTCTCCTCC
GGGCTGTAATTAGCGCTTGGTTTAATGACGGCTCGTTTCTTTTCTGTGGCTGCGTGAAAGCCTTA
AAGGGCTCCGGGAGGGCCCTTTGTGCGGGGGGGAGCGGCTCGGGGGGTGCGTGCGTGTGTGTGTG
CGTGGGGAGCGCCGCGTGCGGCCCGCGCTGCCCGGCGGCTGTGAGCGCTGCGGGCGCGGCGCGGG
GCTTTGTGCGCTCCGCGTGTGCGCGAGGGGAGCGCGGCCGGGGGCGGTGCCCCGCGGTGCGGGGG
GGCTGCGAGGGGAACAAAGGCTGCGTGCGGGGTGTGTGCGTGGGGGGGTGAGCAGGGGGTGTGGG
CGCGGCGGTCGGGCTGTAACCCCCCCCTGCACCCCCCTCCCCGAGTTGCTGAGCACGGCCCGGCT
TCGGGTGCGGGGCTCCGTGCGGGGCGTGGCGCGGGGCTCGCCGTGCCGGGCGGGGGGTGGCGGCA
GGTGGGGGTGCCGGGCGGGGCGGGGCCGCCTCGGGCCGGGGAGGGCTCGGGGGAGGGGCGCGGCG
GCCCCCGGAGCGCCGGCGGCTGTCGAGGCGCGGCGAGCCGCAGCCATTGCCTTTTATGGTAATCG
TGCGAGAGGGCGCAGGGACTTCCTTTGTCCCAAATCTGTGCGGAGCCGAAATCTGGGAGGCGCCG
CCGCACCCCCTCTAGCGGGCGCGGGGCGAAGCGGTGCGGCGCCGGCAGGAAGGAAATGGGCGGGG
AGGGCCTTCGTGCGTCGCCGCGCCGCCGTCCCCTTCTCCCTCTCCAGCCTCGGGGCTGTCCGCGG
GGGGACGGCTGCCTTCGGGGGGGACGGGGCAGGGCGGGGTTCGGCTTCTGGCGTGTGACCGGCGG
CTCTAGAGCCTCTGCTAACCATGTTCATGCCTTCTTCTTTTTCCTACAGCTCCTGGGCAACGTGC
TGGTTATTGTGACCGGTGTTGCGGCCCCGCAGCGCCCGCGCGCTCCTCTCCCCGACTCGGAGCCC
CTCGGCGGCGCCCGGCCCAGGACCCGCCTAGGAGCGCAGGAGCCCCAGCGCAGAGACCCCAACGC
CGAGACCCCCGCCCCGGCCCCGCCGCGCTTCCTCCCGACGCAGTTTAGGACCCTTGTTCGCGAAG
AGGTGGTGTGCGGCTGAGACCCGCGTCCTCAGGACGGTTCCATCAGTGCCTCGATCCTGCCCCAC
TGGAGGAGGAAGGCAGCCCGAACAGCGCTCACCTAACTAACAGCTGCTGAGAGCTGGGTTCCGTG
GCCATGCACCTGGGACTGCCTTGAGAAGCGTGAGCAAACCGCCCAGAGTAGAAGCGCTAGCCACC
ATGGATTGGGGCACGCTGCAGACGATCCTGGGGGGTGTGAACAAACACTCCACCAGCATTGGAAA
GATCTGGCTCACCGTCCTCTTCATTTTTCGCATTATGATCCTCGTTGTGGCTGCAAAGGAGGTGT
GGGGAGATGAGCAGGCCGACTTTGTCTGCAACACCCTGCAGCCAGGCTGCAAGAACGTGTGCTAC
GATCACTACTTCCCCATCTCCCACATCCGGCTATGGGCCCTGCAGCTGATCTTCGTGTCCACGCC
AGCGCTCCTAGTGGCCATGCACGTGGCCTACCGGAGACATGAGAAGAAGAGGAAGTTCATCAAGG
GGGAGATAAAGAGTGAATTTAAGGACATCGAGGAGATCAAAACCCAGAAGGTCCGCATCGAAGGC
TCCCTGTGGTGGACCTACACAAGCAGCATCTTCTTCCGGGTCATCTTCGAAGCCGCCTTCATGTA
CGTCTTCTATGTCATGTACGACGGCTTCTCCATGCAGCGGCTGGTGAAGTGCAACGCCTGGCCTT
GTCCCAACACTGTGGACTGCTTTGTGTCCCGGCCCACGGAGAAGACTGTCTTCACAGTGTTCATG
ATTGCAGTGTCTGGAATTTGCATCCTGCTGAATGTCACTGAATTGTGTTATTTGCTAATTAGATA
TTGTTCTGGGAAGTCAAAAAAGCCAGTTGGATCCCGGGCTGACTACAAAGACCATGACGGTGATT
ATAAAGATCATGACATCGACTACAAGGATGACGATGACAAGTAAGAAATAGACAGCATGAGAGGG
ATGAGGCAACCCGTGCTCAGCTGTCAAGGCTCAGTCGCTAGCATTTCCCAACACAAAGATTCTGA
CCTTAAATGCAACCATTTGAAACCCCTGTAGGCCTCAGGTGAAACTCCAGATGCCACAATGGAGC
TCTGCTCCCCTAAAGCCTCAAAACAAAGGCCTAATTCTATGCCTGTCTTAATTTTCTTTCACTTA
AGTTAGTTCCACTGAGACCCCAGGCTGTTAGGGGTTATTGGTGTAAGGTACTTTCATATTTTAAA
CAGAGGATATCGGCATTTGTTTCTTTCTCTGAGGACAAGAGAAAAAAGCCAGGTTCCACAGAGGA
CACAGAGAAGGTTTGGGTGTCCTCCTGGGGTTCTTTTTGCCAACTTTCCCCACGTTAAAGGTGAA
CATTGGTTCTTTCATTTGCTTTGGAAGTTTTAATCTCTAACAGTGGACAAAGTTACCAGTGCCTT
AAACTCTGTTACACTTTTTGGAAGTGAAAACTTTGTAGTATGATAGGTTATTTTGATGTAAAGAT
GTTCTGGATACCATTATATGTTCCCCCTGTTTCAGAGGCTCAGATTGTAATATGTAAATGGTATG
TCATTCGCTACTATGATTTAATTTGAAATATGGTCTTTTGGTTATGAATACTTTGCAGCACAGCT
GAGAGGCTGTCTGTTGTATTCATTGTGGTCATAGCACCTAACAACATTGTAGCCTCAATCGAGTG
AGACAGACTAGAAGTTCCTAGTGATGGCTTATGATAGCAAATGGCCTCATGTCAAATATTTAGAT
GTAATTTTGTGTAAGAAATACAGACTGGATGTACCACCAACTACTACCTGTAATGACAGGCCTGT
CCAACACATCTCCCTTTTCCATGACTGTGGTAGCCAGCATCGGAAAGAACGCTGATTTAAAGAGG
TCGCTTGGGAATTTTATTGACACAGTACCATTTAATGGGGAGGACAAAATGGGGCAGGGGAGGGA
GAAGTTTCTGTCGTTAAAAACAGATTTGGAAAGACTGGACTCTAAAGTCTGTTGATTAAAGATGA
GCTTTGTCTACTTCAAAAGTTTGTTTGCTTACCCCTTCAGCCTCCAATTTTTTAAGTGAAAATAT
AGCTAATAACATGTGAAAAGAATAGAAGCTAAGGTTTAGATAAATATTGAGCAGATCTATAGGAA
GATTGAACCTGAATATTGCCATTATGCTTGACATGGTTTCCAAAAAATGGTACTCCACATATTTC
AGTGAGGGTAAGTATTTTCCTGTTGTCAAGAATAGCATTGTAAAAGCATTTTGTAATAATAAAGA
ATAGCTTTAATGATATGCTTGTAACTAAAATAATTTTGTAATGTATCAAATACATTTAAAACATT
AAAATATAATCTCTATAATAAGAGCTCGCTGATCAGCCTCGACTGTGCCTTCTAGTTGCCAGCCA
TCTGTTGTTTGCCCCTCCCCCGTGCCTTCCTTGACCCTGGAAGGTGCCACTCCCACTGTCCTTTC
CTAATAAAATGAGGAAATTGCATCGCATTGTCTGAGTAGGTGTCATTCTATTCTGGGGGGTGGGG
TGGGGCAGGACAGCAAGGGGGAGGATTGGGAAGACAATAGCAGGCATGCTGGGGATGCGGTGGGC
TCTATGGAAGCTTGAATTCAGCTGACGTGCCTCGGACCGCTAGGAACCCCTAGTGATGGAGTTGG
CCACTCCCTCTCTGCGCGCTCGCTCGCTCACTGAGGCCGGGCGACCAAAGGTCGCCCGACGCCCG
GGCTTTGCCCGGGCGGCCTCAGTGAGCGAGCGAGCGCGCAG
Exemplary Construct sequence
(SEQ ID NO: 83)
CTGCGCGCTCGCTCGCTCACTGAGGCCGCCCGGGCGTCGGGCGACCTTTGGTCGCCCGGCCTCAG
TGAGCGAGCGAGCGCGCAGAGAGGGAGTGGCCAACTCCATCACTAGGGGTTCCTGCGGCCGCACG
CGTGACATTGATTATTGACTAGTTATTAATAGTAATCAATTACGGGGTCATTAGTTCATAGCCCA
TATATGGAGTTCCGCGTTACATAACTTACGGTAAATGGCCCGCCTGGCTGACCGCCCAACGACCC
CCGCCCATTGACGTCAATAATGACGTATGTTCCCATAGTAACGCCAATAGGGACTTTCCATTGAC
GTCAATGGGTGGACTATTTACGGTAAACTGCCCACTTGGCAGTACATCAAGTGTATCATATGCCA
AGTACGCCCCCTATTGACGTCAATGACGGTAAATGGCCCGCCTGGCATTATGCCCAGTACATGAC
CTTATGGGACTTTCCTACTTGGCAGTACATCTACGTATTAGTCATCGCTATTACCATGGGTCGAG
GTGAGCCCCACGTTCTGCTTCACTCTCCCCATCTCCCCCCCCTCCCCACCCCCAATTTTGTATTT
ATTTATTTTTTAATTATTTTGTGCAGCGATGGGGGCGGGGGGGGGGGGGGCGCGCGCCAGGCGGG
GCGGGGCGGGGCGAGGGGCGGGGCGGGGCGAGGCGGAGAGGTGCGGCGGCAGCCAATCAGAGCGG
CGCGCTCCGAAAGTTTCCTTTTATGGCGAGGCGGCGGCGGCGGCGGCCCTATAAAAAGCGAAGCG
CGCGGCGGGCGGGAGTCGCTGCGTTGCCTTCGCCCCGTGCCCCGCTCCGCGCCGCCTCGCGCCGC
CCGCCCCGGCTCTGACTGACCGCGTTACTCCCACAGGTGAGCGGGCGGGACGGCCCTTCTCCTCC
GGGCTGTAATTAGCGCTTGGTTTAATGACGGCTTGTTTCTTTTCTGTGGCTGCGTGAAAGCCTTG
AGGGGCTCCGGGAGCTAGAGCCTCTGCTAACCATGTTCATGCCTTCTTCTTTTTCCTACAGCTCC
TGGGCAACGTGCTGGTTATTGTGACCGGTGTTGCGGCCCCGCAGCGCCCGCGCGCTCCTCTCCCC
GACTCGGAGCCCCTCGGCGGCGCCCGGCCCAGGACCCGCCTAGGAGCGCAGGAGCCCCAGCGCAG
AGACCCCAACGCCGAGACCCCCGCCCCGGCCCCGCCGCGCTTCCTCCCGACGCAGTTTAGGACCC
TTGTTCGCGAAGAGGTGGTGTGCGGCTGAGACCCGCGTCCTCAGGACGGTTCCATCAGTGCCTCG
ATCCTGCCCCACTGGAGGAGGAAGGCAGCCCGAACAGCGCTCACCTAACTAACAGCTGCTGAGAG
CTGGGTTCCGTGGCCATGCACCTGGGACTGCCTTGAGAAGCGTGAGCAAACCGCCCAGAGTAGAA
GCGCTAGCCACCATGGATTGGGGCACGCTGCAGACGATCCTGGGGGGTGTGAACAAACACTCCAC
CAGCATTGGAAAGATCTGGCTCACCGTCCTCTTCATTTTTCGCATTATGATCCTCGTTGTGGCTG
CAAAGGAGGTGTGGGGAGATGAGCAGGCCGACTTTGTCTGCAACACCCTGCAGCCAGGCTGCAAG
AACGTGTGCTACGATCACTACTTCCCCATCTCCCACATCCGGCTATGGGCCCTGCAGCTGATCTT
CGTGTCCACGCCAGCGCTCCTAGTGGCCATGCACGTGGCCTACCGGAGACATGAGAAGAAGAGGA
AGTTCATCAAGGGGGAGATAAAGAGTGAATTTAAGGACATCGAGGAGATCAAAACCCAGAAGGTC
CGCATCGAAGGCTCCCTGTGGTGGACCTACACAAGCAGCATCTTCTTCCGGGTCATCTTCGAAGC
CGCCTTCATGTACGTCTTCTATGTCATGTACGACGGCTTCTCCATGCAGCGGCTGGTGAAGTGCA
ACGCCTGGCCTTGTCCCAACACTGTGGACTGCTTTGTGTCCCGGCCCACGGAGAAGACTGTCTTC
ACAGTGTTCATGATTGCAGTGTCTGGAATTTGCATCCTGCTGAATGTCACTGAATTGTGTTATTT
GCTAATTAGATATTGTTCTGGGAAGTCAAAAAAGCCAGTTGGATCCCGGGCTGACTACAAAGACC
ATGACGGTGATTATAAAGATCATGACATCGACTACAAGGATGACGATGACAAGTAAGAAATAGAC
AGCATGAGAGGGATGAGGCAACCCGTGCTCAGCTGTCAAGGCTCAGTCGCTAGCATTTCCCAACA
CAAAGATTCTGACCTTAAATGCAACCATTTGAAACCCCTGTAGGCCTCAGGTGAAACTCCAGATG
CCACAATGGAGCTCTGCTCCCCTAAAGCCTCAAAACAAAGGCCTAATTCTATGCCTGTCTTAATT
TTCTTTCACTTAAGTTAGTTCCACTGAGACCCCAGGCTGTTAGGGGTTATTGGTGTAAGGTACTT
TCATATTTTAAACAGAGGATATCGGCATTTGTTTCTTTCTCTGAGGACAAGAGAAAAAAGCCAGG
TTCCACAGAGGACACAGAGAAGGTTTGGGTGTCCTCCTGGGGTTCTTTTTGCCAACTTTCCCCAC
GTTAAAGGTGAACATTGGTTCTTTCATTTGCTTTGGAAGTTTTAATCTCTAACAGTGGACAAAGT
TACCAGTGCCTTAAACTCTGTTACACTTTTTGGAAGTGAAAACTTTGTAGTATGATAGGTTATTT
TGATGTAAAGATGTTCTGGATACCATTATATGTTCCCCCTGTTTCAGAGGCTCAGATTGTAATAT
GTAAATGGTATGTCATTCGCTACTATGATTTAATTTGAAATATGGTCTTTTGGTTATGAATACTT
TGCAGCACAGCTGAGAGGCTGTCTGTTGTATTCATTGTGGTCATAGCACCTAACAACATTGTAGC
CTCAATCGAGTGAGACAGACTAGAAGTTCCTAGTGATGGCTTATGATAGCAAATGGCCTCATGTC
AAATATTTAGATGTAATTTTGTGTAAGAAATACAGACTGGATGTACCACCAACTACTACCTGTAA
TGACAGGCCTGTCCAACACATCTCCCTTTTCCATGACTGTGGTAGCCAGCATCGGAAAGAACGCT
GATTTAAAGAGGTCGCTTGGGAATTTTATTGACACAGTACCATTTAATGGGGAGGACAAAATGGG
GCAGGGGAGGGAGAAGTTTCTGTCGTTAAAAACAGATTTGGAAAGACTGGACTCTAAAGTCTGTT
GATTAAAGATGAGCTTTGTCTACTTCAAAAGTTTGTTTGCTTACCCCTTCAGCCTCCAATTTTTT
AAGTGAAAATATAGCTAATAACATGTGAAAAGAATAGAAGCTAAGGTTTAGATAAATATTGAGCA
GATCTATAGGAAGATTGAACCTGAATATTGCCATTATGCTTGACATGGTTTCCAAAAAATGGTAC
TCCACATATTTCAGTGAGGGTAAGTATTTTCCTGTTGTCAAGAATAGCATTGTAAAAGCATTTTG
TAATAATAAAGAATAGCTTTAATGATATGCTTGTAACTAAAATAATTTTGTAATGTATCAAATAC
ATTTAAAACATTAAAATATAATCTCTATAATAAGAGCTCGCTGATCAGCCTCGACTGTGCCTTCT
AGTTGCCAGCCATCTGTTGTTTGCCCCTCCCCCGTGCCTTCCTTGACCCTGGAAGGTGCCACTCC
CACTGTCCTTTCCTAATAAAATGAGGAAATTGCATCGCATTGTCTGAGTAGGTGTCATTCTATTC
TGGGGGGTGGGGTGGGGCAGGACAGCAAGGGGGAGGATTGGGAAGACAATAGCAGGCATGCTGGG
GATGCGGTGGGCTCTATGGAAGCTTGAATTCAGCTGACGTGCCTCGGACCGCTAGGAACCCCTAG
TGATGGAGTTGGCCACTCCCTCTCTGCGCGCTCGCTCGCTCACTGAGGCCGGGCGACCAAAGGTC
GCCCGACGCCCGGGCTTTGCCCGGGCGGCCTCAGTGAGCGAGCGAGCGCGCAG
Exemplary Construct sequence
(SEQ ID NO: 84)
CTGCGCGCTCGCTCGCTCACTGAGGCCGCCCGGGCGTCGGGCGACCTTTGGTCGCCCGGCCTCAG
TGAGCGAGCGAGCGCGCAGAGAGGGAGTGGCCAACTCCATCACTAGGGGTTCCTGCGGCCGCACG
CGTGACATTGATTATTGACTAGTTATTAATAGTAATCAATTACGGGGTCATTAGTTCATAGCCCA
TATATGGAGTTCCGCGTTACATAACTTACGGTAAATGGCCCGCCTGGCTGACCGCCCAACGACCC
CCGCCCATTGACGTCAATAATGACGTATGTTCCCATAGTAACGCCAATAGGGACTTTCCATTGAC
GTCAATGGGTGGACTATTTACGGTAAACTGCCCACTTGGCAGTACATCAAGTGTATCATATGCCA
AGTACGCCCCCTATTGACGTCAATGACGGTAAATGGCCCGCCTGGCATTATGCCCAGTACATGAC
CTTATGGGACTTTCCTACTTGGCAGTACATCTACGTATTAGTCATCGCTATTACCATGGGTAAGC
TTCCGCAGAATCCTATCAGTTTCCCCCTTTCGTGCTGTGTGCATCGAGCAGGAAGGGGCTTGGCA
GGTTTTACCTGCCCTCTTTCCTTTCTGAAAAGTCTGGGCCTCCTCACCCCGAAAGGAGTCACCTC
CTTGCAGTTCCCCAGTTGCGAAAAGAGGAGGAAGTTGGCTGGGCCGGGGGCCGCGGGGGGCACCC
TCCGCAGATGGCGGGACCCCCCTGCCGGCCATGGCAAAAACGAGGCTTGTCTCTCCCACCGCCCC
CAACCTTAGTCCTTGGCACATTGTTGAAAGTAATTGAATAAAATCGGAAATTCGAGAAGGCGTTC
GTTCGGATTGGTGAGATTTTGAGGGGAGAAAGAAGCGGGGACTTCGCCGGCACCAGCGGCGCCCC
CTCCTCGGCCACCGTTAACCCCCATTCCAGAGGGCACTGCCCCGCCACCCAGCCTAGGTCCCCCT
GCGAGAGCCTCGCGGGCCCGCGCAGCCTCCGCGACTCGAACAGATCTTCAGTCCTTGGAGGAATG
CCTGTTTCTCTAACAATAAAAAATTAAAGAAGCGCTCATAAATGCCAAGTCCTCTCGCACTATGC
GGAGTACAGAGGACAACGACCACAGCCATCCCTGAACCCCGCCCACGGCACAGCGCCGGAGCCGG
GGTCTGGGGCGCCGCTTCCTGGGGGGTCCCGACTCTCAGCCGCCCCCGCTTCACCCGGGCCGCCA
AGGGGCTGGGGGAGGCGGCGCTCGGGGTAACCGGGGGAGACTCAGGGCGCTGGGGGCACTTGGGG
AACTCATGGGGGCTCAAAGGAACTAGGAGATCGGGACCTCGAAGGGGACTTGGGGGGTTCGGGGC
TTTCGGGGGCGGTCGGGGGTTCGCGGACCCGGGAAGCTCTGAGGACCCAGAGGCCGGGCGCGCTC
CGCCCGCGGCGCCGCCCCCTCCGTAACTTTCCCAGTCTCCGAGGGAAGAGGCGGGGTGTGGGGTG
CGGTTAAAAGGCGCCACGGCGGGAGACAGGTCTCCTGGGCAACGTGCTGGTTATTGTGACCGGTG
TTGCGGCCCCGCAGCGCCCGCGCGCTCCTCTCCCCGACTCGGAGCCCCTCGGCGGCGCCCGGCCC
AGGACCCGCCTAGGAGCGCAGGAGCCCCAGCGCAGAGACCCCAACGCCGAGACCCCCGCCCCGGC
CCCGCCGCGCTTCCTCCCGACGCAGTTTAGGACCCTTGTTCGCGAAGAGGTGGTGTGCGGCTGAG
ACCCGCGTCCTCAGGACGGTTCCATCAGTGCCTCGATCCTGCCCCACTGGAGGAGGAAGGCAGCC
CGAACAGCGCTCACCTAACTAACAGCTGCTGAGAGCTGGGTTCCGTGGCCATGCACCTGGGACTG
CCTTGAGAAGCGTGAGCAAACCGCCCAGAGTAGAAGCGCTAGCCACCATGGATTGGGGCACGCTG
CAGACGATCCTGGGGGGTGTGAACAAACACTCCACCAGCATTGGAAAGATCTGGCTCACCGTCCT
CTTCATTTTTCGCATTATGATCCTCGTTGTGGCTGCAAAGGAGGTGTGGGGAGATGAGCAGGCCG
ACTTTGTCTGCAACACCCTGCAGCCAGGCTGCAAGAACGTGTGCTACGATCACTACTTCCCCATC
TCCCACATCCGGCTATGGGCCCTGCAGCTGATCTTCGTGTCCACGCCAGCGCTCCTAGTGGCCAT
GCACGTGGCCTACCGGAGACATGAGAAGAAGAGGAAGTTCATCAAGGGGGAGATAAAGAGTGAAT
TTAAGGACATCGAGGAGATCAAAACCCAGAAGGTCCGCATCGAAGGCTCCCTGTGGTGGACCTAC
ACAAGCAGCATCTTCTTCCGGGTCATCTTCGAAGCCGCCTTCATGTACGTCTTCTATGTCATGTA
CGACGGCTTCTCCATGCAGCGGCTGGTGAAGTGCAACGCCTGGCCTTGTCCCAACACTGTGGACT
GCTTTGTGTCCCGGCCCACGGAGAAGACTGTCTTCACAGTGTTCATGATTGCAGTGTCTGGAATT
TGCATCCTGCTGAATGTCACTGAATTGTGTTATTTGCTAATTAGATATTGTTCTGGGAAGTCAAA
AAAGCCAGTTGGATCCCGGGCTGACTACAAAGACCATGACGGTGATTATAAAGATCATGACATCG
ACTACAAGGATGACGATGACAAGTAAGAAATAGACAGCATGAGAGGGATGAGGCAACCCGTGCTC
AGCTGTCAAGGCTCAGTCGCTAGCATTTCCCAACACAAAGATTCTGACCTTAAATGCAACCATTT
GAAACCCCTGTAGGCCTCAGGTGAAACTCCAGATGCCACAATGGAGCTCTGCTCCCCTAAAGCCT
CAAAACAAAGGCCTAATTCTATGCCTGTCTTAATTTTCTTTCACTTAAGTTAGTTCCACTGAGAC
CCCAGGCTGTTAGGGGTTATTGGTGTAAGGTACTTTCATATTTTAAACAGAGGATATCGGCATTT
GTTTCTTTCTCTGAGGACAAGAGAAAAAAGCCAGGTTCCACAGAGGACACAGAGAAGGTTTGGGT
GTCCTCCTGGGGTTCTTTTTGCCAACTTTCCCCACGTTAAAGGTGAACATTGGTTCTTTCATTTG
CTTTGGAAGTTTTAATCTCTAACAGTGGACAAAGTTACCAGTGCCTTAAACTCTGTTACACTTTT
TGGAAGTGAAAACTTTGTAGTATGATAGGTTATTTTGATGTAAAGATGTTCTGGATACCATTATA
TGTTCCCCCTGTTTCAGAGGCTCAGATTGTAATATGTAAATGGTATGTCATTCGCTACTATGATT
TAATTTGAAATATGGTCTTTTGGTTATGAATACTTTGCAGCACAGCTGAGAGGCTGTCTGTTGTA
TTCATTGTGGTCATAGCACCTAACAACATTGTAGCCTCAATCGAGTGAGACAGACTAGAAGTTCC
TAGTGATGGCTTATGATAGCAAATGGCCTCATGTCAAATATTTAGATGTAATTTTGTGTAAGAAA
TACAGACTGGATGTACCACCAACTACTACCTGTAATGACAGGCCTGTCCAACACATCTCCCTTTT
CCATGACTGTGGTAGCCAGCATCGGAAAGAACGCTGATTTAAAGAGGTCGCTTGGGAATTTTATT
GACACAGTACCATTTAATGGGGAGGACAAAATGGGGCAGGGGAGGGAGAAGTTTCTGTCGTTAAA
AACAGATTTGGAAAGACTGGACTCTAAAGTCTGTTGATTAAAGATGAGCTTTGTCTACTTCAAAA
GTTTGTTTGCTTACCCCTTCAGCCTCCAATTTTTTAAGTGAAAATATAGCTAATAACATGTGAAA
AGAATAGAAGCTAAGGTTTAGATAAATATTGAGCAGATCTATAGGAAGATTGAACCTGAATATTG
CCATTATGCTTGACATGGTTTCCAAAAAATGGTACTCCACATATTTCAGTGAGGGTAAGTATTTT
CCTGTTGTCAAGAATAGCATTGTAAAAGCATTTTGTAATAATAAAGAATAGCTTTAATGATATGC
TTGTAACTAAAATAATTTTGTAATGTATCAAATACATTTAAAACATTAAAATATAATCTCTATAA
TAAGAGCTCGCTGATCAGCCTCGACTGTGCCTTCTAGTTGCCAGCCATCTGTTGTTTGCCCCTCC
CCCGTGCCTTCCTTGACCCTGGAAGGTGCCACTCCCACTGTCCTTTCCTAATAAAATGAGGAAAT
TGCATCGCATTGTCTGAGTAGGTGTCATTCTATTCTGGGGGGTGGGGTGGGGCAGGACAGCAAGG
GGGAGGATTGGGAAGACAATAGCAGGCATGCTGGGGATGCGGTGGGCTCTATGGAAGCTTGAATT
CAGCTGACGTGCCTCGGACCGCTAGGAACCCCTAGTGATGGAGTTGGCCACTCCCTCTCTGCGCG
CTCGCTCGCTCACTGAGGCCGGGCGACCAAAGGTCGCCCGACGCCCGGGCTTTGCCCGGGCGGCC
TCAGTGAGCGAGCGAGCGCGCAG
Exemplary Construct sequence
(SEQ ID NO: 85)
CTGCGCGCTCGCTCGCTCACTGAGGCCGCCCGGGCGTCGGGCGACCTTTGGTCGCCCGGCCTCAG
TGAGCGAGCGAGCGCGCAGAGAGGGAGTGGCCAACTCCATCACTAGGGGTTCCTGCGGCCGCACG
CGTGACATTGATTATTGACTAGTTATTAATAGTAATCAATTACGGGGTCATTAGTTCATAGCCCA
TATATGGAGTTCCGCGTTACATAACTTACGGTAAATGGCCCGCCTGGCTGACCGCCCAACGACCC
CCGCCCATTGACGTCAATAATGACGTATGTTCCCATAGTAACGCCAATAGGGACTTTCCATTGAC
GTCAATGGGTGGACTATTTACGGTAAACTGCCCACTTGGCAGTACATCAAGTGTATCATATGCCA
AGTACGCCCCCTATTGACGTCAATGACGGTAAATGGCCCGCCTGGCATTATGCCCAGTACATGAC
CTTATGGGACTTTCCTACTTGGCAGTACATCTACGTATTAGTCATCGCTATTACCATGGGTGAAC
ATATCCTGGTGTGGAGTAGGGGACGCTGCTCTGACAGAGGCTCGGGGGCCTGAGCTGGCTCTGTG
AGCTGGGGAGGAGGCAGACAGCCAGGCCTTGTCTGCAAGCAGACCTGGCAGCATTGGGCTGGCCG
CCCCCCAGGGCCTCCTCTTCATGCCCAGTGAATGACTCACCTTGGCACAGACACAATGTTCGGGG
TGGGCACAGTGCCTGCTTCCCGCCGCACCCCAGCCCCCCTCAAATGCCTTCCGAGAAGCCCATTG
AGCAGGGGGCTTGCATTGCACCCCAGCCTGACAGCCTGGCATCTTGGGATAAAAGCAGCACAGCC
CCCTAGGGGCTGCCCTTGCTGTGTGGCGCCACCGGCGGTGGAGAACAAGGCTCTATTCAGCCTGT
GCCCAGGAAAGGGGATCAGGGGATGCCCAGGCATGGACAGTGGGTGGCAGGGGGGGAGAGGAGGG
CTGTCTGCTTCCCAGAAGTCCAAGGACACAAATGGGTGAGGGGAGCTCTCCCCATAGCTGGGCTG
CGGCCCAACCCCACCCCCTCAGGCTATGCCAGGGGGTGTTGCCAGGGGCACCCGGGCATCGCCAG
TCTAGCCCACTCCTTCATAAAGCCCTCGCATCCCAGGAGCGAGCAGAGCCAGAGCAGGTTGGAGA
GGAGACGCATCACCTCCGCTGCTCGCCTCCTGGGCAACGTGCTGGTTATTGTGACCGGTGTTGCG
GCCCCGCAGCGCCCGCGCGCTCCTCTCCCCGACTCGGAGCCCCTCGGCGGCGCCCGGCCCAGGAC
CCGCCTAGGAGCGCAGGAGCCCCAGCGCAGAGACCCCAACGCCGAGACCCCCGCCCCGGCCCCGC
CGCGCTTCCTCCCGACGCAGTTTAGGACCCTTGTTCGCGAAGAGGTGGTGTGCGGCTGAGACCCG
CGTCCTCAGGACGGTTCCATCAGTGCCTCGATCCTGCCCCACTGGAGGAGGAAGGCAGCCCGAAC
AGCGCTCACCTAACTAACAGCTGCTGAGAGCTGGGTTCCGTGGCCATGCACCTGGGACTGCCTTG
AGAAGCGTGAGCAAACCGCCCAGAGTAGAAGCGCTAGCCACCATGGATTGGGGCACGCTGCAGAC
GATCCTGGGGGGTGTGAACAAACACTCCACCAGCATTGGAAAGATCTGGCTCACCGTCCTCTTCA
TTTTTCGCATTATGATCCTCGTTGTGGCTGCAAAGGAGGTGTGGGGAGATGAGCAGGCCGACTTT
GTCTGCAACACCCTGCAGCCAGGCTGCAAGAACGTGTGCTACGATCACTACTTCCCCATCTCCCA
CATCCGGCTATGGGCCCTGCAGCTGATCTTCGTGTCCACGCCAGCGCTCCTAGTGGCCATGCACG
TGGCCTACCGGAGACATGAGAAGAAGAGGAAGTTCATCAAGGGGGAGATAAAGAGTGAATTTAAG
GACATCGAGGAGATCAAAACCCAGAAGGTCCGCATCGAAGGCTCCCTGTGGTGGACCTACACAAG
CAGCATCTTCTTCCGGGTCATCTTCGAAGCCGCCTTCATGTACGTCTTCTATGTCATGTACGACG
GCTTCTCCATGCAGCGGCTGGTGAAGTGCAACGCCTGGCCTTGTCCCAACACTGTGGACTGCTTT
GTGTCCCGGCCCACGGAGAAGACTGTCTTCACAGTGTTCATGATTGCAGTGTCTGGAATTTGCAT
CCTGCTGAATGTCACTGAATTGTGTTATTTGCTAATTAGATATTGTTCTGGGAAGTCAAAAAAGC
CAGTTGGATCCCGGGCTGACTACAAAGACCATGACGGTGATTATAAAGATCATGACATCGACTAC
AAGGATGACGATGACAAGTAAGAAATAGACAGCATGAGAGGGATGAGGCAACCCGTGCTCAGCTG
TCAAGGCTCAGTCGCTAGCATTTCCCAACACAAAGATTCTGACCTTAAATGCAACCATTTGAAAC
CCCTGTAGGCCTCAGGTGAAACTCCAGATGCCACAATGGAGCTCTGCTCCCCTAAAGCCTCAAAA
CAAAGGCCTAATTCTATGCCTGTCTTAATTTTCTTTCACTTAAGTTAGTTCCACTGAGACCCCAG
GCTGTTAGGGGTTATTGGTGTAAGGTACTTTCATATTTTAAACAGAGGATATCGGCATTTGTTTC
TTTCTCTGAGGACAAGAGAAAAAAGCCAGGTTCCACAGAGGACACAGAGAAGGTTTGGGTGTCCT
CCTGGGGTTCTTTTTGCCAACTTTCCCCACGTTAAAGGTGAACATTGGTTCTTTCATTTGCTTTG
GAAGTTTTAATCTCTAACAGTGGACAAAGTTACCAGTGCCTTAAACTCTGTTACACTTTTTGGAA
GTGAAAACTTTGTAGTATGATAGGTTATTTTGATGTAAAGATGTTCTGGATACCATTATATGTTC
CCCCTGTTTCAGAGGCTCAGATTGTAATATGTAAATGGTATGTCATTCGCTACTATGATTTAATT
TGAAATATGGTCTTTTGGTTATGAATACTTTGCAGCACAGCTGAGAGGCTGTCTGTTGTATTCAT
TGTGGTCATAGCACCTAACAACATTGTAGCCTCAATCGAGTGAGACAGACTAGAAGTTCCTAGTG
ATGGCTTATGATAGCAAATGGCCTCATGTCAAATATTTAGATGTAATTTTGTGTAAGAAATACAG
ACTGGATGTACCACCAACTACTACCTGTAATGACAGGCCTGTCCAACACATCTCCCTTTTCCATG
ACTGTGGTAGCCAGCATCGGAAAGAACGCTGATTTAAAGAGGTCGCTTGGGAATTTTATTGACAC
AGTACCATTTAATGGGGAGGACAAAATGGGGCAGGGGAGGGAGAAGTTTCTGTCGTTAAAAACAG
ATTTGGAAAGACTGGACTCTAAAGTCTGTTGATTAAAGATGAGCTTTGTCTACTTCAAAAGTTTG
TTTGCTTACCCCTTCAGCCTCCAATTTTTTAAGTGAAAATATAGCTAATAACATGTGAAAAGAAT
AGAAGCTAAGGTTTAGATAAATATTGAGCAGATCTATAGGAAGATTGAACCTGAATATTGCCATT
ATGCTTGACATGGTTTCCAAAAAATGGTACTCCACATATTTCAGTGAGGGTAAGTATTTTCCTGT
TGTCAAGAATAGCATTGTAAAAGCATTTTGTAATAATAAAGAATAGCTTTAATGATATGCTTGTA
ACTAAAATAATTTTGTAATGTATCAAATACATTTAAAACATTAAAATATAATCTCTATAATAAGA
GCTCGCTGATCAGCCTCGACTGTGCCTTCTAGTTGCCAGCCATCTGTTGTTTGCCCCTCCCCCGT
GCCTTCCTTGACCCTGGAAGGTGCCACTCCCACTGTCCTTTCCTAATAAAATGAGGAAATTGCAT
CGCATTGTCTGAGTAGGTGTCATTCTATTCTGGGGGGTGGGGTGGGGCAGGACAGCAAGGGGGAG
GATTGGGAAGACAATAGCAGGCATGCTGGGGATGCGGTGGGCTCTATGGAAGCTTGAATTCAGCT
GACGTGCCTCGGACCGCTAGGAACCCCTAGTGATGGAGTTGGCCACTCCCTCTCTGCGCGCTCGC
TCGCTCACTGAGGCCGGGCGACCAAAGGTCGCCCGACGCCCGGGCTTTGCCCGGGCGGCCTCAGT
GAGCGAGCGAGCGCGCAG
Exemplary Construct sequence
(SEQ ID NO: 86)
CTGCGCGCTCGCTCGCTCACTGAGGCCGCCCGGGCGTCGGGCGACCTTTGGTCGCCCGGCCTCAG
TGAGCGAGCGAGCGCGCAGAGAGGGAGTGGCCAACTCCATCACTAGGGGTTCCTGCGGCCGCACG
CGTGGTGAACATATCCTGGTGTGGAGTAGGGGACGCTGCTCTGACAGAGGCTCGGGGGCCTGAGC
TGGCTCTGTGAGCTGGGGAGGAGGCAGACAGCCAGGCCTTGTCTGCAAGCAGACCTGGCAGCATT
GGGCTGGCCGCCCCCCAGGGCCTCCTCTTCATGCCCAGTGAATGACTCACCTTGGCACAGACACA
ATGTTCGGGGTGGGCACAGTGCCTGCTTCCCGCCGCACCCCAGCCCCCCTCAAATGCCTTCCGAG
AAGCCCATTGAGCAGGGGGCTTGCATTGCACCCCAGCCTGACAGCCTGGCATCTTGGGATAAAAG
CAGCACAGCCCCCTAGGGGCTGCCCTTGCTGTGTGGCGCCACCGGCGGTGGAGAACAAGGCTCTA
TTCAGCCTGTGCCCAGGAAAGGGGATCAGGGGATGCCCAGGCATGGACAGTGGGTGGCAGGGGGG
GAGAGGAGGGCTGTCTGCTTCCCAGAAGTCCAAGGACACAAATGGGTGAGGGGAGCTCTCCCCAT
AGCTGGGCTGCGGCCCAACCCCACCCCCTCAGGCTATGCCAGGGGGTGTTGCCAGGGGCACCCGG
GCATCGCCAGTCTAGCCCACTCCTTCATAAAGCCCTCGCATCCCAGGAGCGAGCAGAGCCAGAGC
AGGTTGGAGAGGAGACGCATCACCTCCGCTGCTCGCCTCCTGGGCAACGTGCTGGTTATTGTGAC
CGGTGTTGCGGCCCCGCAGCGCCCGCGCGCTCCTCTCCCCGACTCGGAGCCCCTCGGCGGCGCCC
GGCCCAGGACCCGCCTAGGAGCGCAGGAGCCCCAGCGCAGAGACCCCAACGCCGAGACCCCCGCC
CCGGCCCCGCCGCGCTTCCTCCCGACGCAGTTTAGGACCCTTGTTCGCGAAGAGGTGGTGTGCGG
CTGAGACCCGCGTCCTCAGGACGGTTCCATCAGTGCCTCGATCCTGCCCCACTGGAGGAGGAAGG
CAGCCCGAACAGCGCTCACCTAACTAACAGCTGCTGAGAGCTGGGTTCCGTGGCCATGCACCTGG
GACTGCCTTGAGAAGCGTGAGCAAACCGCCCAGAGTAGAAGCGCTAGCCACCATGGATTGGGGCA
CGCTGCAGACGATCCTGGGGGGTGTGAACAAACACTCCACCAGCATTGGAAAGATCTGGCTCACC
GTCCTCTTCATTTTTCGCATTATGATCCTCGTTGTGGCTGCAAAGGAGGTGTGGGGAGATGAGCA
GGCCGACTTTGTCTGCAACACCCTGCAGCCAGGCTGCAAGAACGTGTGCTACGATCACTACTTCC
CCATCTCCCACATCCGGCTATGGGCCCTGCAGCTGATCTTCGTGTCCACGCCAGCGCTCCTAGTG
GCCATGCACGTGGCCTACCGGAGACATGAGAAGAAGAGGAAGTTCATCAAGGGGGAGATAAAGAG
TGAATTTAAGGACATCGAGGAGATCAAAACCCAGAAGGTCCGCATCGAAGGCTCCCTGTGGTGGA
CCTACACAAGCAGCATCTTCTTCCGGGTCATCTTCGAAGCCGCCTTCATGTACGTCTTCTATGTC
ATGTACGACGGCTTCTCCATGCAGCGGCTGGTGAAGTGCAACGCCTGGCCTTGTCCCAACACTGT
GGACTGCTTTGTGTCCCGGCCCACGGAGAAGACTGTCTTCACAGTGTTCATGATTGCAGTGTCTG
GAATTTGCATCCTGCTGAATGTCACTGAATTGTGTTATTTGCTAATTAGATATTGTTCTGGGAAG
TCAAAAAAGCCAGTTGGATCCCGGGCTGACTACAAAGACCATGACGGTGATTATAAAGATCATGA
CATCGACTACAAGGATGACGATGACAAGTAAGAAATAGACAGCATGAGAGGGATGAGGCAACCCG
TGCTCAGCTGTCAAGGCTCAGTCGCTAGCATTTCCCAACACAAAGATTCTGACCTTAAATGCAAC
CATTTGAAACCCCTGTAGGCCTCAGGTGAAACTCCAGATGCCACAATGGAGCTCTGCTCCCCTAA
AGCCTCAAAACAAAGGCCTAATTCTATGCCTGTCTTAATTTTCTTTCACTTAAGTTAGTTCCACT
GAGACCCCAGGCTGTTAGGGGTTATTGGTGTAAGGTACTTTCATATTTTAAACAGAGGATATCGG
CATTTGTTTCTTTCTCTGAGGACAAGAGAAAAAAGCCAGGTTCCACAGAGGACACAGAGAAGGTT
TGGGTGTCCTCCTGGGGTTCTTTTTGCCAACTTTCCCCACGTTAAAGGTGAACATTGGTTCTTTC
ATTTGCTTTGGAAGTTTTAATCTCTAACAGTGGACAAAGTTACCAGTGCCTTAAACTCTGTTACA
CTTTTTGGAAGTGAAAACTTTGTAGTATGATAGGTTATTTTGATGTAAAGATGTTCTGGATACCA
TTATATGTTCCCCCTGTTTCAGAGGCTCAGATTGTAATATGTAAATGGTATGTCATTCGCTACTA
TGATTTAATTTGAAATATGGTCTTTTGGTTATGAATACTTTGCAGCACAGCTGAGAGGCTGTCTG
TTGTATTCATTGTGGTCATAGCACCTAACAACATTGTAGCCTCAATCGAGTGAGACAGACTAGAA
GTTCCTAGTGATGGCTTATGATAGCAAATGGCCTCATGTCAAATATTTAGATGTAATTTTGTGTA
AGAAATACAGACTGGATGTACCACCAACTACTACCTGTAATGACAGGCCTGTCCAACACATCTCC
CTTTTCCATGACTGTGGTAGCCAGCATCGGAAAGAACGCTGATTTAAAGAGGTCGCTTGGGAATT
TTATTGACACAGTACCATTTAATGGGGAGGACAAAATGGGGCAGGGGAGGGAGAAGTTTCTGTCG
TTAAAAACAGATTTGGAAAGACTGGACTCTAAAGTCTGTTGATTAAAGATGAGCTTTGTCTACTT
CAAAAGTTTGTTTGCTTACCCCTTCAGCCTCCAATTTTTTAAGTGAAAATATAGCTAATAACATG
TGAAAAGAATAGAAGCTAAGGTTTAGATAAATATTGAGCAGATCTATAGGAAGATTGAACCTGAA
TATTGCCATTATGCTTGACATGGTTTCCAAAAAATGGTACTCCACATATTTCAGTGAGGGTAAGT
ATTTTCCTGTTGTCAAGAATAGCATTGTAAAAGCATTTTGTAATAATAAAGAATAGCTTTAATGA
TATGCTTGTAACTAAAATAATTTTGTAATGTATCAAATACATTTAAAACATTAAAATATAATCTC
TATAATAAGAGCTCGCTGATCAGCCTCGACTGTGCCTTCTAGTTGCCAGCCATCTGTTGTTTGCC
CCTCCCCCGTGCCTTCCTTGACCCTGGAAGGTGCCACTCCCACTGTCCTTTCCTAATAAAATGAG
GAAATTGCATCGCATTGTCTGAGTAGGTGTCATTCTATTCTGGGGGGTGGGGTGGGGCAGGACAG
CAAGGGGGAGGATTGGGAAGACAATAGCAGGCATGCTGGGGATGCGGTGGGCTCTATGGAAGCTT
GAATTCAGCTGACGTGCCTCGGACCGCTAGGAACCCCTAGTGATGGAGTTGGCCACTCCCTCTCT
GCGCGCTCGCTCGCTCACTGAGGCCGGGCGACCAAAGGTCGCCCGACGCCCGGGCTTTGCCCGGG
CGGCCTCAGTGAGCGAGCGAGCGCGCAG
Exemplary Construct sequence
(SEQ ID NO: 87)
CTGCGCGCTCGCTCGCTCACTGAGGCCGCCCGGGCGTCGGGCGACCTTTGGTCGCCCGGCCTCAG
TGAGCGAGCGAGCGCGCAGAGAGGGAGTGGCCAACTCCATCACTAGGGGTTCCTGCGGCCGCACG
CGTGACATTGATTATTGACTAGTTATTAATAGTAATCAATTACGGGGTCATTAGTTCATAGCCCA
TATATGGAGTTCCGCGTTACATAACTTACGGTAAATGGCCCGCCTGGCTGACCGCCCAACGACCC
CCGCCCATTGACGTCAATAATGACGTATGTTCCCATAGTAACGCCAATAGGGACTTTCCATTGAC
GTCAATGGGTGGACTATTTACGGTAAACTGCCCACTTGGCAGTACATCAAGTGTATCATATGCCA
AGTACGCCCCCTATTGACGTCAATGACGGTAAATGGCCCGCCTGGCATTATGCCCAGTACATGAC
CTTATGGGACTTTCCTACTTGGCAGTACATCTACGTATTAGTCATCGCTATTACCATGGGTCGAG
GTGAGCCCCACGTTCTGCTTCACTCTCCCCATCTCCCCCCCCTCCCCACCCCCAATTTTGTATTT
ATTTATTTTTTAATTATTTTGTGCAGCGATGGGGGCGGGGGGGGGGGGGGCGCGCGCCAGGCGGG
GCGGGGCGGGGCGAGGGGCGGGGCGGGGCGAGGCGGAGAGGTGCGGCGGCAGCCAATCAGAGCGG
CGCGCTCCGAAAGTTTCCTTTTATGGCGAGGCGGCGGCGGCGGCGGCCCTATAAAAAGCGAAGCG
CGCGGCGGGCGGGAGTCGCTGCGTTGCCTTCGCCCCGTGCCCCGCTCCGCGCCGCCTCGCGCCGC
CCGCCCCGGCTCTGACTGACCGCGTTACTCCCACAGGTGAGCGGGCGGGACGGCCCTTCTCCTCC
GGGCTGTAATTAGCGCTTGGTTTAATGACGGCTCGTTTCTTTTCTGTGGCTGCGTGAAAGCCTTA
AAGGGCTCCGGGAGGGCCCTTTGTGCGGGGGGGAGCGGCTCGGGGGGTGCGTGCGTGTGTGTGTG
CGTGGGGAGCGCCGCGTGCGGCCCGCGCTGCCCGGCGGCTGTGAGCGCTGCGGGCGCGGCGCGGG
GCTTTGTGCGCTCCGCGTGTGCGCGAGGGGAGCGCGGCCGGGGGCGGTGCCCCGCGGTGCGGGGG
GGCTGCGAGGGGAACAAAGGCTGCGTGCGGGGTGTGTGCGTGGGGGGGTGAGCAGGGGGTGTGGG
CGCGGCGGTCGGGCTGTAACCCCCCCCTGCACCCCCCTCCCCGAGTTGCTGAGCACGGCCCGGCT
TCGGGTGCGGGGCTCCGTGCGGGGCGTGGCGCGGGGCTCGCCGTGCCGGGCGGGGGGTGGCGGCA
GGTGGGGGTGCCGGGCGGGGCGGGGCCGCCTCGGGCCGGGGAGGGCTCGGGGGAGGGGCGCGGCG
GCCCCCGGAGCGCCGGCGGCTGTCGAGGCGCGGCGAGCCGCAGCCATTGCCTTTTATGGTAATCG
TGCGAGAGGGCGCAGGGACTTCCTTTGTCCCAAATCTGTGCGGAGCCGAAATCTGGGAGGCGCCG
CCGCACCCCCTCTAGCGGGCGCGGGGCGAAGCGGTGCGGCGCCGGCAGGAAGGAAATGGGCGGGG
AGGGCCTTCGTGCGTCGCCGCGCCGCCGTCCCCTTCTCCCTCTCCAGCCTCGGGGCTGTCCGCGG
GGGGACGGCTGCCTTCGGGGGGGACGGGGCAGGGCGGGGTTCGGCTTCTGGCGTGTGACCGGCGG
CTCTAGAGCCTCTGCTAACCATGTTCATGCCTTCTTCTTTTTCCTACAGCTCCTGGGCAACGTGC
TGGTTATTGTGACCGGTGTTGCGGCCCCGCAGCGCCCGCGCGCTCCTCTCCCCGACTCGGAGCCC
CTCGGCGGCGCCCGGCCCAGGACCCGCCTAGGAGCGCAGGAGCCCCAGCGCAGAGACCCCAACGC
CGAGACCCCCGCCCCGGCCCCGCCGCGCTTCCTCCCGACGCAGTTTAGGACCCTTGTTCGCGAAG
AGGTGGTGTGCGGCTGAGACCCGCGTCCTCAGGACGGTTCCATCAGTGCCTCGATCCTGCCCCAC
TGGAGGAGGAAGGCAGCCCGAACAGCGCTCACCTAACTAACAGCTGCTGAGAGCTGGGTTCCGTG
GCCATGCACCTGGGACTGCCTTGAGAAGCGTGAGCAAACCGCCCAGAGTAGAAGCGCTAGCCACC
ATGGATTGGGGCACGCTGCAGACGATCCTGGGGGGTGTGAACAAACACTCCACCAGCATTGGAAA
GATCTGGCTCACCGTCCTCTTCATTTTTCGCATTATGATCCTCGTTGTGGCTGCAAAGGAGGTGT
GGGGAGATGAGCAGGCCGACTTTGTCTGCAACACCCTGCAGCCAGGCTGCAAGAACGTGTGCTAC
GATCACTACTTCCCCATCTCCCACATCCGGCTATGGGCCCTGCAGCTGATCTTCGTGTCCACGCC
AGCGCTCCTAGTGGCCATGCACGTGGCCTACCGGAGACATGAGAAGAAGAGGAAGTTCATCAAGG
GGGAGATAAAGAGTGAATTTAAGGACATCGAGGAGATCAAAACCCAGAAGGTCCGCATCGAAGGC
TCCCTGTGGTGGACCTACACAAGCAGCATCTTCTTCCGGGTCATCTTCGAAGCCGCCTTCATGTA
CGTCTTCTATGTCATGTACGACGGCTTCTCCATGCAGCGGCTGGTGAAGTGCAACGCCTGGCCTT
GTCCCAACACTGTGGACTGCTTTGTGTCCCGGCCCACGGAGAAGACTGTCTTCACAGTGTTCATG
ATTGCAGTGTCTGGAATTTGCATCCTGCTGAATGTCACTGAATTGTGTTATTTGCTAATTAGATA
TTGTTCTGGGAAGTCAAAAAAGCCAGTTGGATCCCGGGCTGACTACAAAGACCATGACGGTGATT
ATAAAGATCATGACATCGACTACAAGGATGACGATGACAAGTAAGAGCTCAGTGTGAGTTCTACC
ATTGCCAAACTCGAGCAGTGAATTCTACCAGTGCCATAGGATCCAGTGTGAGTTCTACCATTGCC
AAAGGTACCCAGTGAATTCTACCAGTGCCATAGTTAACCGCATTGCCCAGTTGTTAGATTAAGAA
ATAGACAGCATGAGAGGGATGAGGCAACCCGTGCTCAGCTGTCAAGGCTCAGTCGCTAGCATTTC
CCAACACAAAGATTCTGACCTTAAATGCAACCATTTGAAACCCCTGTAGGCCTCAGGTGAAACTC
CAGATGCCACAATGGAGCCTCTGCTCCCCTAAAGCCTCAAAACAAAGGCCTAATTCTATGCCTGT
CTTAATTTTCTTTCACTTAAGTTAGTTCCACTGAGACCCCAGGCTGTTAGGGGTTATTGGTGTAA
GGTACTTTCATATTTTAAACAGAGGATATCGGCATTTGTTTCTTTCTCTGAGGACAAGAGAAAAA
AGCCAGGTTCCACAGAGGACACAGAGAAGGTTTGGGTGTCCTCCTGGGGTTCTTTTTGCCAACTT
TCCCCACGTTAAAGGTGAACATTGGTTCTTTCATTTGCTTTGGAAGTTTTAATCTCTAACAGTGG
ACAAAGTTACCAGTGCCTTAAACTCTGTTACACTTTTTGGAAGTGAAAACTTTGTAGTATGATAG
GTTATTTTGATGTAAAGATGTTCTGGATACCATTATATGTTCCCCCTGTTTCAGAGGCTCAGATT
GTAATATGTAAATGGTATGTCATTCGCTACTATGATTTAATTTGAAATATGGTCTTTTGGTTATG
AATACTTTGCAGCACAGCTGAGAGGCTGTCTGTTGTATTCATTGTGGTCATAGCACCTAACAACA
TTGTAGCCTCAATCGAGTGAGACAGACTAGAAGTTCCTAGTGATGGCTTATGATAGCAAATGGCC
TCATGTCAAATATTTAGATGTAATTTTGTGTAAGAAATACAGACTGGATGTACCACCAACTACTA
CCTGTAATGACAGGCCTGTCCAACACATCTCCCTTTTCCATGACTGTGGTAGCCAGCATCGGAAA
GAACGCTGATTTAAAGAGGTCGCTTGGGAATTTTATTGACACAGTACCATTTAATGGGGAGGACA
AAATGGGGCAGGGGAGGGAGAAGTTTCTGTCGTTAAAAACAGATTTGGAAAGACTGGACTCTAAA
GTCTGTTGATTAAAGATGAGCTTTGTCTACTTCAAAAGTTTGTTTGCTTACCCCTTCAGCCTCCA
ATTTTTTAAGTGAAAATATAGCTAATAACATGTGAAAAGAATAGAAGCTAAGGTTTAGATAAATA
TTGAGCAGATCTATAGGAAGATTGAACCTGAATATTGCCATTATGCTTGACGCTGATCAGCCTCG
ACTGTGCCTTCTAGTTGCCAGCCATCTGTTGTTTGCCCCTCCCCCGTGCCTTCCTTGACCCTGGA
AGGTGCCACTCCCACTGTCCTTTCCTAATAAAATGAGGAAATTGCATCGCATTGTCTGAGTAGGT
GTCATTCTATTCTGGGGGGTGGGGTGGGGCAGGACAGCAAGGGGGAGGATTGGGAAGACAATAGC
AGGCATGCTGGGGATGCGGTGGGCTCTATGGAAGCTTGAATTCAGCTGACGTGCCTCGGACCGCT
AGGAACCCCTAGTGATGGAGTTGGCCACTCCCTCTCTGCGCGCTCGCTCGCTCACTGAGGCCGGG
CGACCAAAGGTCGCCCGACGCCCGGGCTTTGCCCGGGCGGCCTCAGTGAGCGAGCGAGCGCGCAG
Exemplary Construct sequence
(SEQ ID NO: 88)
CTGCGCGCTCGCTCGCTCACTGAGGCCGCCCGGGCGTCGGGCGACCTTTGGTCGCCCGGCCTCAG
TGAGCGAGCGAGCGCGCAGAGAGGGAGTGGCCAACTCCATCACTAGGGGTTCCTGCGGCCGCACG
CGTCTTCTTCTGGAGTCTTTTCTGGAATAATTCTGGGAGTGGGCTCAGCCTGCGGGAGAGTAACA
TTTTTATAACTTGATAGATGTAGCTGAGATGCCTCCCAGAGGGGAGACCCGCCTCTCCTCCGGCA
GCTGTGCACGTAGGCTTGTTCCCAGCAGCCTGGCCAGGGTGGTCCACCTGGTGTTTCTCATCTTC
TTTCCCCGGAGCGCTGACTCCTGCGCGTCCTCTTGGAAGACTCTTGACAGGACGGGTGTTTTATG
GGTGTGATTCAGTGTCCTCTTGCATCAGTTCAATGTGGTGGTGTTCAATCAACCCTTGTAGCGTT
AGCAAAATTTGCTCAAGTCATTCCGCAGGAATGTCTGTGTCTTGCTTCCAAGAAAGCTTGTAAGT
GCCGGCAACAGGCCAAGCAGCTCACAAACCTGACCACAAGCCTGTGAGTAATTGTGGGGCAGCAC
TTAGCAGTCTTTTATTTTCGACTTATTAAAGTCTCATCTTGGCCTCACCTTCTCCCTGGAAGGTG
GCGTGGGTGGGAACCACTGGGTCAGATCTTTTTCACCCTTGCCGTGGAGCCAGTTTCCTGTTGCA
TGTGGGGGAAGCAACATGTGGTGAAGAGTATAGAAAACGAAAACATGTGGGTACAGTATGTATAA
GTGGAGGGAACAAACTCATAATTCCAACTAGTTTCTCATGAGAGACTCATGAATCATTGTGGTAG
TTCTCAATATAAACTTAATCTAGGCCGGATGTGGTGGCTCACACCTGTAATCTCAGCACTCTGGG
TGGATCACTTGAGGTCAGGAGTTTGAGACCAGTCTGACCAACATGGAGAAACCCCATCGCTACTA
AAAATACAAAATTATCCAGATGTGGTGGCTCACACCTGTAATCCCAGCACTTTGGGAGGCTGAGG
CGGGTGGATCACTTGAGGTCAGGAGTTTGAGACCAGCCTGACCAACATGGAGAAACTGTGTCTCT
ACTAAAAATACAAAATTAGCTGGGCGTGGTGACGCATGCCTGTAATCCCAGCTATTTGGAGGCCG
AAGCAGGAAGCTTCCGCAGAATCCTATCAGTTTCCCCCTTTCGTGCTGTGTGCATCGAGCAGGAA
GGGGCTTGGCAGGTTTTACCTGCCCTCTTTCCTTTCTGAAAAGTCTGGGCCTCCTCACCCCGAAA
GGAGTCACCTCCTTGCAGTTCCCCAGTTGCGAAAAGAGGAGGAAGTTGGCTGGGCCGGGGGCCGC
GGGGGGCACCCTCCGCAGATGGCGGGACCCCCCTGCCGGCCATGGCAAAAACGAGGCTTGTCTCT
CCCACCGCCCCCAACCTTAGTCCTTGGCACATTGTTGAAAGTAATTGAATAAAATCGGAAATTCG
AGAAGGCGTTCGTTCGGATTGGTGAGATTTTGAGGGGAGAAAGAAGCGGGGACTTCGCCGGCACC
AGCGGCGCCCCCTCCTCGGCCACCGTTAACCCCCATTCCAGAGGGCACTGCCCCGCCACCCAGCC
TAGGTCCCCCTGCGAGAGCCTCGCGGGCCCGCGCAGCCTCCGCGACTCGAACAGATCTTCAGTCC
TTGGAGGAATGCCTGTTTCTCTAACAATAAAAAATTAAAGAAGCGCTCATAAATGCCAAGTCCTC
TCGCACTATGCGGAGTACAGAGGACAACGACCACAGCCATCCCTGAACCCCGCCCACGGCACAGC
GCCGGAGCCGGGGTCTGGGGCGCCGCTTCCTGGGGGGTCCCGACTCTCAGCCGCCCCCGCTTCAC
CCGGGCCGCCAAGGGGCTGGGGGAGGCGGCGCTCGGGGTAACCGGGGGAGACTCAGGGCGCTGGG
GGCACTTGGGGAACTCATGGGGGCTCAAAGGAACTAGGAGATCGGGACCTCGAAGGGGACTTGGG
GGGTTCGGGGCTTTCGGGGGCGGTCGGGGGTTCGCGGACCCGGGAAGCTCTGAGGACCCAGAGGC
CGGGCGCGCTCCGCCCGCGGCGCCGCCCCCTCCGTAACTTTCCCAGTCTCCGAGGGAAGAGGCGG
GGTGTGGGGTGCGGTTAAAAGGCGCCACGGCGGGAGACAGGTGTTGCGGCCCCGCAGCGCCCGCG
CGCTCCTCTCCCCGACTCGGAGCCCCTCGGCGGCGCCCGGCCCAGGACCCGCCTAGGAGCGCAGG
AGCCCCAGCGCAGAGACCCCAACGCCGAGACCCCCGCCCCGGCCCCGCCGCGCTTCCTCCCGACG
CAGAGCAAACCGCCCAGAGTAGAAGACCGGTCGCTAGCCACCATGGATTGGGGCACGCTGCAGAC
GATCCTGGGGGGTGTGAACAAACACTCCACCAGCATTGGAAAGATCTGGCTCACCGTCCTCTTCA
TTTTTCGCATTATGATCCTCGTTGTGGCTGCAAAGGAGGTGTGGGGAGATGAGCAGGCCGACTTT
GTCTGCAACACCCTGCAGCCAGGCTGCAAGAACGTGTGCTACGATCACTACTTCCCCATCTCCCA
CATCCGGCTATGGGCCCTGCAGCTGATCTTCGTGTCCACGCCAGCGCTCCTAGTGGCCATGCACG
TGGCCTACCGGAGACATGAGAAGAAGAGGAAGTTCATCAAGGGGGAGATAAAGAGTGAATTTAAG
GACATCGAGGAGATCAAAACCCAGAAGGTCCGCATCGAAGGCTCCCTGTGGTGGACCTACACAAG
CAGCATCTTCTTCCGGGTCATCTTCGAAGCCGCCTTCATGTACGTCTTCTATGTCATGTACGACG
GCTTCTCCATGCAGCGGCTGGTGAAGTGCAACGCCTGGCCTTGTCCCAACACTGTGGACTGCTTT
GTGTCCCGGCCCACGGAGAAGACTGTCTTCACAGTGTTCATGATTGCAGTGTCTGGAATTTGCAT
CCTGCTGAATGTCACTGAATTGTGTTATTTGCTAATTAGATATTGTTCTGGGAAGTCAAAAAAGC
CAGTTGGATCCCGGGCTGACTACAAAGACCATGACGGTGATTATAAAGATCATGACATCGACTAC
AAGGATGACGATGACAAGTAAGAAATAGACAGCATGAGAGGGATGAGGCAACCCGTGCTCAGCTG
TCAAGGCTCAGTCGCTAGCATTTCCCAACACAAAGATTCTGACCTTAAATGCAACCATTTGAAAC
CCCTGTAGGCCTCAGGTGAAACTCCAGATGCCACAATGGAGCTCTGCTCCCCTAAAGCCTCAAAA
CAAAGGCCTAATTCTATGCCTGTCTTAATTTTCTTTCACTTAAGTTAGTTCCACTGAGACCCCAG
GCTGTTAGGGGTTATTGGTGTAAGGTACTTTCATATTTTAAACAGAGGATATCGGCATTTGTTTC
TTTCTCTGAGGACAAGAGAAAAAAGCCAGGTTCCACAGAGGACACAGAGAAGGTTTGGGTGTCCT
CCTGGGGTTCTTTTTGCCAACTTTCCCCACGTTAAAGGTGAACATTGGTTCTTTCATTTGCTTTG
GAAGTTTTAATCTCTAACAGTGGACAAAGTTACCAGTGCCTTAAACTCTGTTACACTTTTTGGAA
GTGAAAACTTTGTAGTATGATAGGTTATTTTGATGTAAAGATGTTCTGGATACCATTATATGTTC
CCCCTGTTTCAGAGGCTCAGATTGTAATATGTAAATGGTATGTCATTCGCTACTATGATTTAATT
TGAAATATGGTCTTTTGGTTATGAATACTTTGCAGCACAGCTGAGAGGCTGTCTGTTGTATTCAT
TGTGGTCATAGCACCTAACAACATTGTAGCCTCAATCGAGTGAGACAGACTAGAAGTTCCTAGTG
ATGGCTTATGATAGCAAATGGCCTCATGTCAAATATTTAGATGTAATTTTGTGTAAGAAATACAG
ACTGGATGTACCACCAACTACTACCTGTAATGACAGGCCTGTCCAACACATCTCCCTTTTCCATG
ACTGTGGTAGCCAGCATCGGAAAGAACGCTGATTTAAAGAGGTCGCTTGGGAATTTTATTGACAC
AGTACCATTTAATGGGGAGGACAAAATGGGGCAGGGGAGGGAGAAGTTTCTGTCGTTAAAAACAG
ATTTGGAAAGACTGGACTCTAAAGTCTGTTGATTAAAGATGAGCTTTGTCTACTTCAAAAGTTTG
TTTGCTTACCCCTTCAGCCTCCAATTTTTTAAGTGAAAATATAGCTAATAACATGTGAAAAGAAT
AGAAGCTAAGGTTTAGATAAATATTGAGCAGATCTATAGGAAGATTGAACCTGAATATTGCCATT
ATGCTTGACATGGTTTCCAAAAAATGGTACTCCACATATTTCAGTGAGGGTAAGTATTTTCCTGT
TGTCAAGAATAGCATTGTAAAAGCATTTTGTAATAATAAAGAATAGCTTTAATGATATGCTTGTA
ACTAAAATAATTTTGTAATGTATCAAATACATTTAAAACATTAAAATATAATCTCTATAATAAAA
GCTTGAATTCAGCTGACGTGCCTCGGACCGCTAGGAACCCCTAGTGATGGAGTTGGCCACTCCCT
CTCTGCGCGCTCGCTCGCTCACTGAGGCCGGGCGACCAAAGGTCGCCCGACGCCCGGGCTTTGCC
CGGGCGGCCTCAGTGAGCGAGCGAGCGCGCAG
Exemplary Construct sequence
(SEQ ID NO: 94)
CCTGCAGGCAGCTGCGCGCTCGCTCGCTCACTGAGGCCGCCCGGGCGTCGGGCGACC
TTTGGTCGCCCGGCCTCAGTGAGCGAGCGAGCGCGCAGAGAGGGAGTGGCCAACTC
CATCACTAGGGGTTCCTGCGGCCGCACGCGTGGTCCACAGGTAACTCCGTCGGCGTC
CACAGGGGGGCAGGAGATACCATACTGCACAGTTGTACGTCTTCCATCTGTTTGGTG
TAGAAAAATCTAACCACTACAAGAATGCCACGGGCACTGTGGCAGACAGAAGCAGC
GCTACGCCGCATCGCCTTTCAGCGTGCAGGCCCAGGAATGAGCGAGGCAGTGGGCG
GGGAAGACAGGCACGGGGAATCTGGGGACAGATAAAGGAAACTCGTGATGGGGCG
AGGCTGGGCTGAAGAGAAACAGATTGGGGTAGAGCTGCAAAGGGAGGGGTCCACT
GGAAGGCGAGGGGGGAGGCCGGGAAGAGAGAGGGTGGGAAGGCAGTGTGAGATGG
GAGGGCAGTGTGAGAAGAAAAGCAGGCTGGGGAAGAGGGATTGGAATGCAGAAGG
AACTTGGGGAAGGAGGAAGTCCTGCAGGCGGGAGGGAAAGAAGAGAGGGGGAGCA
GCTAAAGTCTGCGTCAGAAGAGGTTGGGGACTGCGAGAGGAGAGGCTGGGGCCTGC
AGGGGAGCGCAGCAGCTTTTAGCATCGATCCAAACTCTAAAGACTCGTGGCCTTTGC
CTGACCTCGAGGGTCGGGAATAGACGCCTGTCTTTGTGGAGAGCGATACCCAACCG
AGAAAATGGGGCTGTTCCGAGCTGGGCCCTGCGCCTGGCCCAGGGCGAGGCTTCTCT
GGCTCCGGGCTGGCCCCTGAGGGGCAGCACGCAGCCTGCAGCAGAGGCGCCTGCTC
CAAGCTGTCTCTTGGGGGCGCCGCCGCCGCTTCCCTCCTCCGGGGCCGCTCGCTCCC
AGGAAAGTGGAGGCGGCTGGCGAGGACCGAGAGCCGGGGCCGCGCTGCGGAGGGA
CCACACCTCCGGGAGTTCGAGGGGGACCCTGGCGCGGCGGGCCAGCCTTTCGGGCC
GGCAGCGCCCGCCTTCCCCCGGTCAGCGCTTGCGGCCCGCGCCGCGCGCACCGCCCG
GCAACCCCGCGCGCGTCCCGCGGGGGCGCTGCGTCTTCCTGCCACACCGGCGCACCG
CGGCCCCTCTCCCCCACACCTCCGGCCCGCACCACCCGGCTCTCCTCCCACCCTCCCC
ACCCCTCCTCTGCCCTCCCTCCCCATTCCTCCCCTCCCGGCGAGGGGCGGGAGGGGG
CGTGGCGGGGCCGGGGTTTGTGTGGCTGGGACCCGGCTCCTCAAGCTCTGAGGACCC
AGAGGCCGGGCGCGCTCCGCCCGCGGCGCCGCCCCCTCCGTAACTTTCCCAGTCTCC
GAGGGAAGAGGCGGGGTGTGGGGTGCGGTTAAAAGGCGCCACGGCGGGAGACAGG
TCTCACCGGTGTGTCACCGTTGCGGCCCCGCAGCGCCCGCGCGCTCCTCTCCCCGAC
TCGGAGCCCCTCGGCGGCGCCCGGCCCAGGACCCGCCTAGGAGCGCAGGAGCCCCA
GCGCAGAGACCCCAACGCCGAGACCCCCGCCCCGGCCCCGCCGCGCTTCCTCCCGAC
GCAGTTTAGGACCCTTGTTCGCGAAGAGGTGGTGTGCGGCTGAGACCCGCGTCCTCA
GGACGGTTCCATCAGTGCCTCGATCCTGCCCCACTGGAGGAGGAAGGCAGCCCGAA
CAGCGCTCACCTAACTAACAGCTGCTGAGAGCTGGGTTCCGTGGCCATGCACCTGGG
ACTGCCTTGAGAAGCGTGAGCAAACCGCCCAGAGTAGAAGCGCTAGCCACCATGGA
TTGGGGCACGCTGCAGACGATCCTGGGGGGTGTGAACAAACACTCCACCAGCATTG
GAAAGATCTGGCTCACCGTCCTCTTCATTTTTCGCATTATGATCCTCGTTGTGGCTGC
AAAGGAGGTGTGGGGAGATGAGCAGGCCGACTTTGTCTGCAACACCCTGCAGCCAG
GCTGCAAGAACGTGTGCTACGATCACTACTTCCCCATCTCCCACATCCGGCTATGGG
CCCTGCAGCTGATCTTCGTGTCCACGCCAGCGCTCCTAGTGGCCATGCACGTGGCCT
ACCGGAGACATGAGAAGAAGAGGAAGTTCATCAAGGGGGAGATAAAGAGTGAATT
TAAGGACATCGAGGAGATCAAAACCCAGAAGGTCCGCATCGAAGGCTCCCTGTGGT
GGACCTACACAAGCAGCATCTTCTTCCGGGTCATCTTCGAAGCCGCCTTCATGTACG
TCTTCTATGTCATGTACGACGGCTTCTCCATGCAGCGGCTGGTGAAGTGCAACGCCT
GGCCTTGTCCCAACACTGTGGACTGCTTTGTGTCCCGGCCCACGGAGAAGACTGTCT
TCACAGTGTTCATGATTGCAGTGTCTGGAATTTGCATCCTGCTGAATGTCACTGAATT
GTGTTATTTGCTAATTAGATATTGTTCTGGGAAGTCAAAAAAGCCAGTTGGATCCCG
GGCTGACTACAAAGACCATGACGGTGATTATAAAGATCATGACATCGACTACAAGG
ATGACGATGACAAGTAAGAAATAGACAGCATGAGAGGGATGAGGCAACCCGTGCTC
AGCTGTCAAGGCTCAGTCGCTAGCATTTCCCAACACAAAGATTCTGACCTTAAATGC
AACCATTTGAAACCCCTGTAGGCCTCAGGTGAAACTCCAGATGCCACAATGGAGCTC
TGCTCCCCTAAAGCCTCAAAACAAAGGCCTAATTCTATGCCTGTCTTAATTTTCTTTC
ACTTAAGTTAGTTCCACTGAGACCCCAGGCTGTTAGGGGTTATTGGTGTAAGGTACT
TTCATATTTTAAACAGAGGATATCGGCATTTGTTTCTTTCTCTGAGGACAAGAGAAA
AAAGCCAGGTTCCACAGAGGACACAGAGAAGGTTTGGGTGTCCTCCTGGGGTTCTTT
TTGCCAACTTTCCCCACGTTAAAGGTGAACATTGGTTCTTTCATTTGCTTTGGAAGTT
TTAATCTCTAACAGTGGACAAAGTTACCAGTGCCTTAAACTCTGTTACACTTTTTGGA
AGTGAAAACTTTGTAGTATGATAGGTTATTTTGATGTAAAGATGTTCTGGATACCATT
ATATGTTCCCCCTGTTTCAGAGGCTCAGATTGTAATATGTAAATGGTATGTCATTCGC
TACTATGATTTAATTTGAAATATGGTCTTTTGGTTATGAATACTTTGCAGCACAGCTG
AGAGGCTGTCTGTTGTATTCATTGTGGTCATAGCACCTAACAACATTGTAGCCTCAA
TCGAGTGAGACAGACTAGAAGTTCCTAGTGATGGCTTATGATAGCAAATGGCCTCAT
GTCAAATATTTAGATGTAATTTTGTGTAAGAAATACAGACTGGATGTACCACCAACT
ACTACCTGTAATGACAGGCCTGTCCAACACATCTCCCTTTTCCATGACTGTGGTAGCC
AGCATCGGAAAGAACGCTGATTTAAAGAGGTCGCTTGGGAATTTTATTGACACAGTA
CCATTTAATGGGGAGGACAAAATGGGGCAGGGGAGGGAGAAGTTTCTGTCGTTAAA
AACAGATTTGGAAAGACTGGACTCTAAAGTCTGTTGATTAAAGATGAGCTTTGTCTA
CTTCAAAAGTTTGTTTGCTTACCCCTTCAGCCTCCAATTTTTTAAGTGAAAATATAGC
TAATAACATGTGAAAAGAATAGAAGCTAAGGTTTAGATAAATATTGAGCAGATCTA
TAGGAAGATTGAACCTGAATATTGCCATTATGCTTGACATGGTTTCCAAAAAATGGT
ACTCCACATATTTCAGTGAGGGTAAGTATTTTCCTGTTGTCAAGAATAGCATTGTAA
AAGCATTTTGTAATAATAAAGAATAGCTTTAATGATATGCTTGTAACTAAAATAATT
TTGTAATGTATCAAATACATTTAAAACATTAAAATATAATCTCTATAATAAGAGCTC
GCTGATCAGCCTCGACTGTGCCTTCTAGTTGCCAGCCATCTGTTGTTTGCCCCTCCCC
CGTGCCTTCCTTGACCCTGGAAGGTGCCACTCCCACTGTCCTTTCCTAATAAAATGAG
GAAATTGCATCGCATTGTCTGAGTAGGTGTCATTCTATTCTGGGGGGTGGGGTGGGG
CAGGACAGCAAGGGGGAGGATTGGGAAGACAATAGCAGGCATGCTGGGGATGCGG
TGGGCTCTATGGAAGCTTGAATTCAGCTGACGTGCCTCGGACCGCTAGGAACCCCTA
GTGATGGAGTTGGCCACTCCCTCTCTGCGCGCTCGCTCGCTCACTGAGGCCGGGCGA
CCAAAGGTCGCCCGACGCCCGGGCTTTGCCCGGGCGGCCTCAGTGAGCGAGCGAGC
GCGCAGCTGCCTGCAGGGGCGCCTGATGCGGTATTTTCTCCTTACGCATCTGTGCGG
TATTTCACACCGCATACGTCAAAGCAACCATAGTACGCGCCCTGTAGCGGCGCATTA
AGCGCGGCGGGTGTGGTGGTTACGCGCAGCGTGACCGCTACACTTGCCAGCGCCCTA
GCGCCCGCTCCTTTCGCTTTCTTCCCTTCCTTTCTCGCCACGTTCGCCGGCTTTCCCCG
TCAAGCTCTAAATCGGGGGCTCCCTTTAGGGTTCCGATTTAGTGCTTTACGGCACCTC
GACCCCAAAAAACTTGATTTGGGTGATGGTTCACGTAGTGGGCCATCGCCCTGATAG
ACGGTTTTTCGCCCTTTGACGTTGGAGTCCACGTTCTTTAATAGTGGACTCTTGTTCC
AAACTGGAACAACACTCAACCCTATCTCGGGCTATTCTTTTGATTTATAAGGGATTTT
GCCGATTTCGGCCTATTGGTTAAAAAATGAGCTGATTTAACAAAAATTTAACGCGAA
TTTTAACAAAATATTAACGTTTACAATTTTATGGTGCACTCTCAGTACAATCTGCTCT
GATGCCGCATAGTTAAGCCAGCCCCGACACCCGCCAACACCCGCTGACGCGCCCTG
ACGGGCTTGTCTGCTCCCGGCATCCGCTTACAGACAAGCTGTGACCGTCTCCGGGAG
CTGCATGTGTCAGAGGTTTTCACCGTCATCACCGAAACGCGCGAGACGAAAGGGCCT
CGTGATACGCCTATTTTTATAGGTTAATGTCATGAACAATAAAACTGTCTGCTTACAT
AAACAGTAATACAAGGGGTGTTATGAGCCATATTCAACGGGAAACGTCGAGGCCGC
GATTAAATTCCAACATGGATGCTGATTTATATGGGTATAAATGGGCTCGCGATAATG
TCGGGCAATCAGGTGCGACAATCTATCGCTTGTATGGGAAGCCCGATGCGCCAGAGT
TGTTTCTGAAACATGGCAAAGGTAGCGTTGCCAATGATGTTACAGATGAGATGGTCA
GACTAAACTGGCTGACGGAATTTATGCCTCTTCCGACCATCAAGCATTTTATCCGTA
CTCCTGATGATGCATGGTTACTCACCACTGCGATCCCCGGAAAAACAGCATTCCAGG
TATTAGAAGAATATCCTGATTCAGGTGAAAATATTGTTGATGCGCTGGCAGTGTTCC
TGCGCCGGTTGCATTCGATTCCTGTTTGTAATTGTCCTTTTAACAGCGATCGCGTATT
TCGTCTCGCTCAGGCGCAATCACGAATGAATAACGGTTTGGTTGATGCGAGTGATTT
TGATGACGAGCGTAATGGCTGGCCTGTTGAACAAGTCTGGAAAGAAATGCATAAAC
TTTTGCCATTCTCACCGGATTCAGTCGTCACTCATGGTGATTTCTCACTTGATAACCT
TATTTTTGACGAGGGGAAATTAATAGGTTGTATTGATGTTGGACGAGTCGGAATCGC
AGACCGATACCAGGATCTTGCCATCCTATGGAACTGCCTCGGTGAGTTTTCTCCTTCA
TTACAGAAACGGCTTTTTCAAAAATATGGTATTGATAATCCTGATATGAATAAATTG
CAGTTTCATTTGATGCTCGATGAGTTTTTCTAATCTCATGACCAAAATCCCTTAACGT
GAGTTTTCGTTCCACTGAGCGTCAGACCCCGTAGAAAAGATCAAAGGATCTTCTTGA
GATCCTTTTTTTCTGCGCGTAATCTGCTGCTTGCAAACAAAAAAACCACCGCTACCA
GCGGTGGTTTGTTTGCCGGATCAAGAGCTACCAACTCTTTTTCCGAAGGTAACTGGC
TTCAGCAGAGCGCAGATACCAAATACTGTCCTTCTAGTGTAGCCGTAGTTAGGCCAC
CACTTCAAGAACTCTGTAGCACCGCCTACATACCTCGCTCTGCTAATCCTGTTACCAG
TGGCTGCTGCCAGTGGCGATAAGTCGTGTCTTACCGGGTTGGACTCAAGACGATAGT
TACCGGATAAGGCGCAGCGGTCGGGCTGAACGGGGGGTTCGTGCACACAGCCCAGC
TTGGAGCGAACGACCTACACCGAACTGAGATACCTACAGCGTGAGCTATGAGAAAG
CGCCACGCTTCCCGAAGGGAGAAAGGCGGACAGGTATCCGGTAAGCGGCAGGGTCG
GAACAGGAGAGCGCACGAGGGAGCTTCCAGGGGGAAACGCCTGGTATCTTTATAGT
CCTGTCGGGTTTCGCCACCTCTGACTTGAGCGTCGATTTTTGTGATGCTCGTCAGGGG
GGCGGAGCCTATGGAAAAACGCCAGCAACGCGGCCTTTTTACGGTTCCTGGCCTTTT
GCTGGCCTTTTGCTCACATGT

TABLE 3
Components of Construct Sequence (SEQ ID NO: 94)

| Components | Position in construct |
|---|---|
| 5′ITR | 12-130 |
| Cloning site | 131-147 |
| GDF6 promoter | 148-1335 |
| hGJB2 minimal promoter | 1336-1463 |
| Cloning site | 1464-1472 |
| Synthetic barcode | 1473-1480 |
| 5′UTR | 1481-1842 |
| GJB2 (exon2) | 1854-2531 |
| 3xFLAG | 2544-2609 |
| 3′UTR (exon2) | 2613-4019 |
| bGHpA | 4041-4265 |
| Cloning site | 4266-4299 |
| 3′ITR | 4300-4429 |

Exemplary Construct sequence
(SEQ ID NO: 97)
CCTGCAGGCAGCTGCGCGCTCGCTCGCTCACTGAGGCCGCCCGGGCGTCGGGCGACCTTTGGTCGCCCGGCCTCAGTGAGC
GAGCGAGCGCGCAGAGAGGGAGTGGCCAACTCCATCACTAGGGGTTCCTGCGGCCGCACGCGTGGTAAGAAACTTGCCCGA
GTTTACACAGCTAGTAAATGGTTGCATTAGTCAGGACAGCTAGCCTATATTACAATAACAACCCTCTCAAATCCTAATGGC
TTAAAACAACAGAGGTTTAATTTATACTCATTAGCTGTTCAAGGCAGGAGGCTCTATTCTCTAATCCATACAGTCACTCAG
GATCCAGGCTGGTGGAGACCCTGCCATATTGTAGCCTCACCATTTAAAACATGAAGAAGATAGAAAGTGAGGAGTCATGTA
GGTTTTGTTCCGTTGCCTCAGGCTAGGAGTGACAGGTCACTTCATCTCACTCACAGCTCACTGCCCACAACTAGTCACTTG
TGACTGTGCGAGTTAAGCTTCTGTGTGTGAAGGAAGGAAAAGAGAATGGGATAAAGGTGAACATCAGCAGGCTCTACCACA
GTAGTTTGAACCAAGACTTGAGCCTAGGTCATGTGGCTTCAGAATCTTTGCTCTTAATCACACTAAACAGCCTCTGTAAGT
CATCTTTCCTTCATCCAGTGCCTAAGAACATGCAGTCCAATGCCCTCATCCTTCAGAAGAACTTGAGTGAACTCAGAGAAA
TTGAGTAGAGTGCCACAGCATGCCCAAGGCCACACACCCTGAGGTTGGCAGTAGGTCCTGAGTTAGAGTTGTCATTTCTTG
GCTCCCCTGGTAGTAGTGGAAAGGTAAGGTTTTGACATACTAGTTGGATGACCACGGGCAGGTCACTTAAATTGTCTAAGC
ATCGTTTGACCCTTGTAAGAATTAAATGAAATAGCACCTGTAAAAGTGTCTGCACGGACTTACTGCTGTTAGTTTTGTTCC
TTTCTTCCTGTTGTCACTGCACTTCCCTGCCTGTTACCCAGGCCATGCAGACCAGCCAGGCCTTCGACTTACAGTGCGGAT
AAGATTCCAAATCTCCACGGCTGGTTTCCATGCTTTCTTCCAGGCTTCTGAGGACCCTGTGCTCTGGTTTCTTCTATTTCT
TTTCTATTACTTTTCTGTTACTCTTGAGCACACTTGCTGGAAGCAATATGCATCCAGTTCTCCCTCTCTTGCCTCATTACA
CTTTGCAGAACAACTCCAATCCCTTCCAACCAAGTAGTCCCTTTGAATTTCTTGTCACCCAAGGAATCTCTCTGACAGGGG
TCTTTGTTAGGGTCACACCCCAGGAGATGGTTGATTATGGCTGAGTCCAGCCTGGAATGATGGGGGTTGGGGGCAGCTTGG
GTAGATGACTCAGTAAATCAAACAGAACAATGAAAGGAGGTCATGCTTGTCCATCTGCATTATTGAAGACAGCCATAAATG
GCCTTACCCCAGAGCGGGTCTGTCACACCTGGAGAGCTGATCTGACCTCTCCAAGACCCCTGCAACTGAGTGTTCTGGGAT
CTGTCCTGCAACAAGTGCCTCGAGATTTGTAGGTGGGGGCCCAGAGGGAGGGGGTCTGCAGACGAAGGGGGCAGGTTTTGC
GGGGCACTTAGGGTTCTCATAGGTTGTAGTCACGAGCTCCAAGCTCTGAGGACCCAGAGGCCGGGCGCGCTCCGCCCGCGG
CGCCGCCCCCTCCGTAACTTTCCCAGTCTCCGAGGGAAGAGGCGGGGTGTGGGGTGCGGTTAAAAGGCGCCACGGCGGGAG
ACAGGTCTCACCGGTCACAACCTGTTGCGGCCCCGCAGCGCCCGCGCGCTCCTCTCCCCGACTCGGAGCCCCTCGGCGGCG
CCCGGCCCAGGACCCGCCTAGGAGCGCAGGAGCCCCAGCGCAGAGACCCCAACGCCGAGACCCCCGCCCCGGCCCCGCCGC
GCTTCCTCCCGACGCAGTTTAGGACCCTTGTTCGCGAAGAGGTGGTGTGCGGCTGAGACCCGCGTCCTCAGGACGGTTCCA
TCAGTGCCTCGATCCTGCCCCACTGGAGGAGGAAGGCAGCCCGAACAGCGCTCACCTAACTAACAGCTGCTGAGAGCTGGG
TTCCGTGGCCATGCACCTGGGACTGCCTTGAGAAGCGTGAGCAAACCGCCCAGAGTAGAAGCGCTAGCCACCATGGATTGG
GGCACGCTGCAGACGATCCTGGGGGGTGTGAACAAACACTCCACCAGCATTGGAAAGATCTGGCTCACCGTCCTCTTCATT
TTTCGCATTATGATCCTCGTTGTGGCTGCAAAGGAGGTGTGGGGAGATGAGCAGGCCGACTTTGTCTGCAACACCCTGCAG
CCAGGCTGCAAGAACGTGTGCTACGATCACTACTTCCCCATCTCCCACATCCGGCTATGGGCCCTGCAGCTGATCTTCGTG
TCCACGCCAGCGCTCCTAGTGGCCATGCACGTGGCCTACCGGAGACATGAGAAGAAGAGGAAGTTCATCAAGGGGGAGATA
AAGAGTGAATTTAAGGACATCGAGGAGATCAAAACCCAGAAGGTCCGCATCGAAGGCTCCCTGTGGTGGACCTACACAAGC
AGCATCTTCTTCCGGGTCATCTTCGAAGCCGCCTTCATGTACGTCTTCTATGTCATGTACGACGGCTTCTCCATGCAGCGG
CTGGTGAAGTGCAACGCCTGGCCTTGTCCCAACACTGTGGACTGCTTTGTGTCCCGGCCCACGGAGAAGACTGTCTTCACA
GTGTTCATGATTGCAGTGTCTGGAATTTGCATCCTGCTGAATGTCACTGAATTGTGTTATTTGCTAATTAGATATTGTTCT
GGGAAGTCAAAAAAGCCAGTTGGATCCCGGGCTGACTACAAAGACCATGACGGTGATTATAAAGATCATGACATCGACTAC
AAGGATGACGATGACAAGTAAGAAATAGACAGCATGAGAGGGATGAGGCAACCCGTGCTCAGCTGTCAAGGCTCAGTCGCT
AGCATTTCCCAACACAAAGATTCTGACCTTAAATGCAACCATTTGAAACCCCTGTAGGCCTCAGGTGAAACTCCAGATGCC
ACAATGGAGCTCTGCTCCCCTAAAGCCTCAAAACAAAGGCCTAATTCTATGCCTGTCTTAATTTTCTTTCACTTAAGTTAG
TTCCACTGAGACCCCAGGCTGTTAGGGGTTATTGGTGTAAGGTACTTTCATATTTTAAACAGAGGATATCGGCATTTGTTT
CTTTCTCTGAGGACAAGAGAAAAAAGCCAGGTTCCACAGAGGACACAGAGAAGGTTTGGGTGTCCTCCTGGGGTTCTTTTT
GCCAACTTTCCCCACGTTAAAGGTGAACATTGGTTCTTTCATTTGCTTTGGAAGTTTTAATCTCTAACAGTGGACAAAGTT
ACCAGTGCCTTAAACTCTGTTACACTTTTTGGAAGTGAAAACTTTGTAGTATGATAGGTTATTTTGATGTAAAGATGTTCT
GGATACCATTATATGTTCCCCCTGTTTCAGAGGCTCAGATTGTAATATGTAAATGGTATGTCATTCGCTACTATGATTTAA
TTTGAAATATGGTCTTTTGGTTATGAATACTTTGCAGCACAGCTGAGAGGCTGTCTGTTGTATTCATTGTGGTCATAGCAC
CTAACAACATTGTAGCCTCAATCGAGTGAGACAGACTAGAAGTTCCTAGTGATGGCTTATGATAGCAAATGGCCTCATGTC
AAATATTTAGATGTAATTTTGTGTAAGAAATACAGACTGGATGTACCACCAACTACTACCTGTAATGACAGGCCTGTCCAA
CACATCTCCCTTTTCCATGACTGTGGTAGCCAGCATCGGAAAGAACGCTGATTTAAAGAGGTCGCTTGGGAATTTTATTGA
CACAGTACCATTTAATGGGGAGGACAAAATGGGGCAGGGGAGGGAGAAGTTTCTGTCGTTAAAAACAGATTTGGAAAGACT
GGACTCTAAAGTCTGTTGATTAAAGATGAGCTTTGTCTACTTCAAAAGTTTGTTTGCTTACCCCTTCAGCCTCCAATTTTT
TAAGTGAAAATATAGCTAATAACATGTGAAAAGAATAGAAGCTAAGGTTTAGATAAATATTGAGCAGATCTATAGGAAGAT
TGAACCTGAATATTGCCATTATGCTTGACATGGTTTCCAAAAAATGGTACTCCACATATTTCAGTGAGGGTAAGTATTTTC
CTGTTGTCAAGAATAGCATTGTAAAAGCATTTTGTAATAATAAAGAATAGCTTTAATGATATGCTTGTAACTAAAATAATT
TTGTAATGTATCAAATACATTTAAAACATTAAAATATAATCTCTATAATAAGAGCTCGCTGATCAGCCTCGACTGTGCCTT
CTAGTTGCCAGCCATCTGTTGTTTGCCCCTCCCCCGTGCCTTCCTTGACCCTGGAAGGTGCCACTCCCACTGTCCTTTCCT
AATAAAATGAGGAAATTGCATCGCATTGTCTGAGTAGGTGTCATTCTATTCTGGGGGGTGGGGTGGGGCAGGACAGCAAGG
GGGAGGATTGGGAAGACAATAGCAGGCATGCTGGGGATGCGGTGGGCTCTATGGAAGCTTGAATTCAGCTGACGTGCCTCG
GACCGCTAGGAACCCCTAGTGATGGAGTTGGCCACTCCCTCTCTGCGCGCTCGCTCGCTCACTGAGGCCGGGCGACCAAAG
GTCGCCCGACGCCCGGGCTTTGCCCGGGCGGCCTCAGTGAGCGAGCGAGCGCGCAGCTGCCTGCAGGGGCGCCTGATGCGG
TATTTTCTCCTTACGCATCTGTGCGGTATTTCACACCGCATACGTCAAAGCAACCATAGTACGCGCCCTGTAGCGGCGCAT
TAAGCGCGGCGGGTGTGGTGGTTACGCGCAGCGTGACCGCTACACTTGCCAGCGCCCTAGCGCCCGCTCCTTTCGCTTTCT
TCCCTTCCTTTCTCGCCACGTTCGCCGGCTTTCCCCGTCAAGCTCTAAATCGGGGGCTCCCTTTAGGGTTCCGATTTAGTG
CTTTACGGCACCTCGACCCCAAAAAACTTGATTTGGGTGATGGTTCACGTAGTGGGCCATCGCCCTGATAGACGGTTTTTC
GCCCTTTGACGTTGGAGTCCACGTTCTTTAATAGTGGACTCTTGTTCCAAACTGGAACAACACTCAACCCTATCTCGGGCT
ATTCTTTTGATTTATAAGGGATTTTGCCGATTTCGGCCTATTGGTTAAAAAATGAGCTGATTTAACAAAAATTTAACGCGA
ATTTTAACAAAATATTAACGTTTACAATTTTATGGTGCACTCTCAGTACAATCTGCTCTGATGCCGCATAGTTAAGCCAGC
CCCGACACCCGCCAACACCCGCTGACGCGCCCTGACGGGCTTGTCTGCTCCCGGCATCCGCTTACAGACAAGCTGTGACCG
TCTCCGGGAGCTGCATGTGTCAGAGGTTTTCACCGTCATCACCGAAACGCGCGAGACGAAAGGGCCTCGTGATACGCCTAT
TTTTATAGGTTAATGTCATGAACAATAAAACTGTCTGCTTACATAAACAGTAATACAAGGGGTGTTATGAGCCATATTCAA
CGGGAAACGTCGAGGCCGCGATTAAATTCCAACATGGATGCTGATTTATATGGGTATAAATGGGCTCGCGATAATGTCGGG
CAATCAGGTGCGACAATCTATCGCTTGTATGGGAAGCCCGATGCGCCAGAGTTGTTTCTGAAACATGGCAAAGGTAGCGTT
GCCAATGATGTTACAGATGAGATGGTCAGACTAAACTGGCTGACGGAATTTATGCCTCTTCCGACCATCAAGCATTTTATC
CGTACTCCTGATGATGCATGGTTACTCACCACTGCGATCCCCGGAAAAACAGCATTCCAGGTATTAGAAGAATATCCTGAT
TCAGGTGAAAATATTGTTGATGCGCTGGCAGTGTTCCTGCGCCGGTTGCATTCGATTCCTGTTTGTAATTGTCCTTTTAAC
AGCGATCGCGTATTTCGTCTCGCTCAGGCGCAATCACGAATGAATAACGGTTTGGTTGATGCGAGTGATTTTGATGACGAG
CGTAATGGCTGGCCTGTTGAACAAGTCTGGAAAGAAATGCATAAACTTTTGCCATTCTCACCGGATTCAGTCGTCACTCAT
GGTGATTTCTCACTTGATAACCTTATTTTTGACGAGGGGAAATTAATAGGTTGTATTGATGTTGGACGAGTCGGAATCGCA
GACCGATACCAGGATCTTGCCATCCTATGGAACTGCCTCGGTGAGTTTTCTCCTTCATTACAGAAACGGCTTTTTCAAAAA
TATGGTATTGATAATCCTGATATGAATAAATTGCAGTTTCATTTGATGCTCGATGAGTTTTTCTAATCTCATGACCAAAAT
CCCTTAACGTGAGTTTTCGTTCCACTGAGCGTCAGACCCCGTAGAAAAGATCAAAGGATCTTCTTGAGATCCTTTTTTTCT
GCGCGTAATCTGCTGCTTGCAAACAAAAAAACCACCGCTACCAGCGGTGGTTTGTTTGCCGGATCAAGAGCTACCAACTCT
TTTTCCGAAGGTAACTGGCTTCAGCAGAGCGCAGATACCAAATACTGTCCTTCTAGTGTAGCCGTAGTTAGGCCACCACTT
CAAGAACTCTGTAGCACCGCCTACATACCTCGCTCTGCTAATCCTGTTACCAGTGGCTGCTGCCAGTGGCGATAAGTCGTG
TCTTACCGGGTTGGACTCAAGACGATAGTTACCGGATAAGGCGCAGCGGTCGGGCTGAACGGGGGGTTCGTGCACACAGCC
CAGCTTGGAGCGAACGACCTACACCGAACTGAGATACCTACAGCGTGAGCTATGAGAAAGCGCCACGCTTCCCGAAGGGAG
AAAGGCGGACAGGTATCCGGTAAGCGGCAGGGTCGGAACAGGAGAGCGCACGAGGGAGCTTCCAGGGGGAAACGCCTGGTA
TCTTTATAGTCCTGTCGGGTTTCGCCACCTCTGACTTGAGCGTCGATTTTTGTGATGCTCGTCAGGGGGGCGGAGCCTATG
GAAAAACGCCAGCAACGCGGCCTTTTTACGGTTCCTGGCCTTTTGCTGGCCTTTTGCTCACATGT

TABLE 4
Components of Construct Sequence (SEQ ID NO: 97)

| Components | Position in construct |
|---|---|
| 5′ITR | 12-130 |
| Cloning site | 131-147 |
| IGFBP2 promoter | 148-1660 |
| hGJB2 minimal promoter | 1661-1788 |
| Cloning site | 1789-1797 |
| Synthetic barcode | 1798-1805 |
| 5′UTR | 1806-2167 |
| GJB2 (exon2) | 2179-2856 |
| 3xFLAG | 2869-2934 |
| 3′UTR (exon2) | 2938-4344 |
| bGHpA | 4366-4590 |
| Cloning site | 4591-4624 |
| 3′ITR | 4625-4754 |

Exemplary Construct sequence
(SEQ ID NO: 100)
CCTGCAGGCAGCTGCGCGCTCGCTCGCTCACTGAGGCCGCCCGGGCGTCGGGCGACCTTTGGTCGCCCGGCCTCAGTGAGCGAGC
GAGCGCGCAGAGAGGGAGTGGCCAACTCCATCACTAGGGGTTCCTGCGGCCGCACGCGTGGTCCCATGGCTCTGTTAAAATCAAA
GAAACATCTTTTCCAACAGCCCTTTCAAACTCCTCATCGCATCTCACTGGCTGATTCAGTCATTTAAACCTGCTTCTCCCTAAAG
CTGATCACTGGCTAAGCTAATAGGGTTTCCGGGATTGGTTTAGCCTGATACTAATCCAGGTCTACCTTCAGGAGCCAGACCAAAC
TGCCTATTGGCATTGCATTCTTGCAGTAGGGAGGGGAGGTATGGATGGTGTGGAGTCCACCACAAGGTCCATGCCAGTCTTTGCT
GAACCAGCATCAGACTCCATCAAGCAACAGATGAGAGGTTCCATGATAAAGTGGCCCTCAGCAATCCCCATCCATTGCTGTCTAG
GAAGAACAGTGCTTGTACACAGGTTTAGGACCTCAGTCTTGGCTGTAATCTTCTGGTTTACTTTGCCAGCACCAAACAGAAGGAA
AGAAAGGGCTCAAATTTGACCAAATAAATTATGCTTCTCCTTCCAGAGATAACCTTGAGTCCTGTCTAGGAAGATATTAGAATTG
TAAAGAAAAAAAAAATTACTCCTTATCCTATGGCAAGTGGAGTCTATGTCTACTTCAGCTGAAATTAAATCCTGTCCATAATAGA
TGACCCTTGCTCAAGCTGGCCAGAAGCCATACCAACCAGCACGAAGGTTAAAACTATTATTAGTTTTTTCTGTGATTTTCATTTT
CAGGCCAAGTTTTAGAACAATAAGATTTTAAGAATAGGAAGTAAGTAAGATTTCTGCATATCCTGTTCTCTTAGTCAGCTGAATT
TTTTTTTTTTTTTTTTTAGTCCTAACTCAGCCTCCCAAAGTGCTGGGATTACAGGCGTGAGCCACCGCACCAAGCCTGGAATCTA
TGTCTTACAGTTATGAGAATCAACAGCTAGCTCATTATGGGCAAGGTGATGTCACTCTGGCTTCTCAATGAAAATGGCATTTCTC
CCTTGGAAAAGGTCATAGCCAGTCAGTCAGTCAGTCACGGGAGCGCAGCGGCTTCTAGGGGTGAGTGGGACCCACGCGGCCCCAC
CTGCTCCTCCCGCGCGCGGCCCCACCCCCCTGCCCCGCCCCGCCTGGTTTATAGAAGCTCTGAGGACCCAGAGGCCGGGCGCGCT
CCGCCCGCGGCGCCGCCCCCTCCGTAACTTTCCCAGTCTCCGAGGGAAGAGGCGGGGTGTGGGGTGCGGTTAAAAGGCGCCACGG
CGGGAGACAGGTCTCACCGGTCGTGTGTTGTTGCGGCCCCGCAGCGCCCGCGCGCTCCTCTCCCCGACTCGGAGCCCCTCGGCGG
CGCCCGGCCCAGGACCCGCCTAGGAGCGCAGGAGCCCCAGCGCAGAGACCCCAACGCCGAGACCCCCGCCCCGGCCCCGCCGCGC
TTCCTCCCGACGCAGTTTAGGACCCTTGTTCGCGAAGAGGTGGTGTGCGGCTGAGACCCGCGTCCTCAGGACGGTTCCATCAGTG
CCTCGATCCTGCCCCACTGGAGGAGGAAGGCAGCCCGAACAGCGCTCACCTAACTAACAGCTGCTGAGAGCTGGGTTCCGTGGCC
ATGCACCTGGGACTGCCTTGAGAAGCGTGAGCAAACCGCCCAGAGTAGAAGCGCTAGCCACCATGGATTGGGGCACGCTGCAGAC
GATCCTGGGGGGTGTGAACAAACACTCCACCAGCATTGGAAAGATCTGGCTCACCGTCCTCTTCATTTTTCGCATTATGATCCTC
GTTGTGGCTGCAAAGGAGGTGTGGGGAGATGAGCAGGCCGACTTTGTCTGCAACACCCTGCAGCCAGGCTGCAAGAACGTGTGCT
ACGATCACTACTTCCCCATCTCCCACATCCGGCTATGGGCCCTGCAGCTGATCTTCGTGTCCACGCCAGCGCTCCTAGTGGCCAT
GCACGTGGCCTACCGGAGACATGAGAAGAAGAGGAAGTTCATCAAGGGGGAGATAAAGAGTGAATTTAAGGACATCGAGGAGATC
AAAACCCAGAAGGTCCGCATCGAAGGCTCCCTGTGGTGGACCTACACAAGCAGCATCTTCTTCCGGGTCATCTTCGAAGCCGCCT
TCATGTACGTCTTCTATGTCATGTACGACGGCTTCTCCATGCAGCGGCTGGTGAAGTGCAACGCCTGGCCTTGTCCCAACACTGT
GGACTGCTTTGTGTCCCGGCCCACGGAGAAGACTGTCTTCACAGTGTTCATGATTGCAGTGTCTGGAATTTGCATCCTGCTGAAT
GTCACTGAATTGTGTTATTTGCTAATTAGATATTGTTCTGGGAAGTCAAAAAAGCCAGTTGGATCCCGGGCTGACTACAAAGACC
ATGACGGTGATTATAAAGATCATGACATCGACTACAAGGATGACGATGACAAGTAAGAAATAGACAGCATGAGAGGGATGAGGCA
ACCCGTGCTCAGCTGTCAAGGCTCAGTCGCTAGCATTTCCCAACACAAAGATTCTGACCTTAAATGCAACCATTTGAAACCCCTG
TAGGCCTCAGGTGAAACTCCAGATGCCACAATGGAGCTCTGCTCCCCTAAAGCCTCAAAACAAAGGCCTAATTCTATGCCTGTCT
TAATTTTCTTTCACTTAAGTTAGTTCCACTGAGACCCCAGGCTGTTAGGGGTTATTGGTGTAAGGTACTTTCATATTTTAAACAG
AGGATATCGGCATTTGTTTCTTTCTCTGAGGACAAGAGAAAAAAGCCAGGTTCCACAGAGGACACAGAGAAGGTTTGGGTGTCCT
CCTGGGGTTCTTTTTGCCAACTTTCCCCACGTTAAAGGTGAACATTGGTTCTTTCATTTGCTTTGGAAGTTTTAATCTCTAACAG
TGGACAAAGTTACCAGTGCCTTAAACTCTGTTACACTTTTTGGAAGTGAAAACTTTGTAGTATGATAGGTTATTTTGATGTAAAG
ATGTTCTGGATACCATTATATGTTCCCCCTGTTTCAGAGGCTCAGATTGTAATATGTAAATGGTATGTCATTCGCTACTATGATT
TAATTTGAAATATGGTCTTTTGGTTATGAATACTTTGCAGCACAGCTGAGAGGCTGTCTGTTGTATTCATTGTGGTCATAGCACC
TAACAACATTGTAGCCTCAATCGAGTGAGACAGACTAGAAGTTCCTAGTGATGGCTTATGATAGCAAATGGCCTCATGTCAAATA
TTTAGATGTAATTTTGTGTAAGAAATACAGACTGGATGTACCACCAACTACTACCTGTAATGACAGGCCTGTCCAACACATCTCC
CTTTTCCATGACTGTGGTAGCCAGCATCGGAAAGAACGCTGATTTAAAGAGGTCGCTTGGGAATTTTATTGACACAGTACCATTT
AATGGGGAGGACAAAATGGGGCAGGGGAGGGAGAAGTTTCTGTCGTTAAAAACAGATTTGGAAAGACTGGACTCTAAAGTCTGTT
GATTAAAGATGAGCTTTGTCTACTTCAAAAGTTTGTTTGCTTACCCCTTCAGCCTCCAATTTTTTAAGTGAAAATATAGCTAATA
ACATGTGAAAAGAATAGAAGCTAAGGTTTAGATAAATATTGAGCAGATCTATAGGAAGATTGAACCTGAATATTGCCATTATGCT
TGACATGGTTTCCAAAAAATGGTACTCCACATATTTCAGTGAGGGTAAGTATTTTCCTGTTGTCAAGAATAGCATTGTAAAAGCA
TTTTGTAATAATAAAGAATAGCTTTAATGATATGCTTGTAACTAAAATAATTTTGTAATGTATCAAATACATTTAAAACATTAAA
ATATAATCTCTATAATAAGAGCTCGCTGATCAGCCTCGACTGTGCCTTCTAGTTGCCAGCCATCTGTTGTTTGCCCCTCCCCCGT
GCCTTCCTTGACCCTGGAAGGTGCCACTCCCACTGTCCTTTCCTAATAAAATGAGGAAATTGCATCGCATTGTCTGAGTAGGTGT
CATTCTATTCTGGGGGGTGGGGTGGGGCAGGACAGCAAGGGGGAGGATTGGGAAGACAATAGCAGGCATGCTGGGGATGCGGTGG
GCTCTATGGAAGCTTGAATTCAGCTGACGTGCCTCGGACCGCTAGGAACCCCTAGTGATGGAGTTGGCCACTCCCTCTCTGCGCG
CTCGCTCGCTCACTGAGGCCGGGCGACCAAAGGTCGCCCGACGCCCGGGCTTTGCCCGGGCGGCCTCAGTGAGCGAGCGAGCGCG
CAGCTGCCTGCAGGGGCGCCTGATGCGGTATTTTCTCCTTACGCATCTGTGCGGTATTTCACACCGCATACGTCAAAGCAACCAT
AGTACGCGCCCTGTAGCGGCGCATTAAGCGCGGCGGGTGTGGTGGTTACGCGCAGCGTGACCGCTACACTTGCCAGCGCCCTAGC
GCCCGCTCCTTTCGCTTTCTTCCCTTCCTTTCTCGCCACGTTCGCCGGCTTTCCCCGTCAAGCTCTAAATCGGGGGCTCCCTTTA
GGGTTCCGATTTAGTGCTTTACGGCACCTCGACCCCAAAAAACTTGATTTGGGTGATGGTTCACGTAGTGGGCCATCGCCCTGAT
AGACGGTTTTTCGCCCTTTGACGTTGGAGTCCACGTTCTTTAATAGTGGACTCTTGTTCCAAACTGGAACAACACTCAACCCTAT
CTCGGGCTATTCTTTTGATTTATAAGGGATTTTGCCGATTTCGGCCTATTGGTTAAAAAATGAGCTGATTTAACAAAAATTTAAC
GCGAATTTTAACAAAATATTAACGTTTACAATTTTATGGTGCACTCTCAGTACAATCTGCTCTGATGCCGCATAGTTAAGCCAGC
CCCGACACCCGCCAACACCCGCTGACGCGCCCTGACGGGCTTGTCTGCTCCCGGCATCCGCTTACAGACAAGCTGTGACCGTCTC
CGGGAGCTGCATGTGTCAGAGGTTTTCACCGTCATCACCGAAACGCGCGAGACGAAAGGGCCTCGTGATACGCCTATTTTTATAG
GTTAATGTCATGAACAATAAAACTGTCTGCTTACATAAACAGTAATACAAGGGGTGTTATGAGCCATATTCAACGGGAAACGTCG
AGGCCGCGATTAAATTCCAACATGGATGCTGATTTATATGGGTATAAATGGGCTCGCGATAATGTCGGGCAATCAGGTGCGACAA
TCTATCGCTTGTATGGGAAGCCCGATGCGCCAGAGTTGTTTCTGAAACATGGCAAAGGTAGCGTTGCCAATGATGTTACAGATGA
GATGGTCAGACTAAACTGGCTGACGGAATTTATGCCTCTTCCGACCATCAAGCATTTTATCCGTACTCCTGATGATGCATGGTTA
CTCACCACTGCGATCCCCGGAAAAACAGCATTCCAGGTATTAGAAGAATATCCTGATTCAGGTGAAAATATTGTTGATGCGCTGG
CAGTGTTCCTGCGCCGGTTGCATTCGATTCCTGTTTGTAATTGTCCTTTTAACAGCGATCGCGTATTTCGTCTCGCTCAGGCGCA
ATCACGAATGAATAACGGTTTGGTTGATGCGAGTGATTTTGATGACGAGCGTAATGGCTGGCCTGTTGAACAAGTCTGGAAAGAA
ATGCATAAACTTTTGCCATTCTCACCGGATTCAGTCGTCACTCATGGTGATTTCTCACTTGATAACCTTATTTTTGACGAGGGGA
AATTAATAGGTTGTATTGATGTTGGACGAGTCGGAATCGCAGACCGATACCAGGATCTTGCCATCCTATGGAACTGCCTCGGTGA
GTTTTCTCCTTCATTACAGAAACGGCTTTTTCAAAAATATGGTATTGATAATCCTGATATGAATAAATTGCAGTTTCATTTGATG
CTCGATGAGTTTTTCTAATCTCATGACCAAAATCCCTTAACGTGAGTTTTCGTTCCACTGAGCGTCAGACCCCGTAGAAAAGATC
AAAGGATCTTCTTGAGATCCTTTTTTTCTGCGCGTAATCTGCTGCTTGCAAACAAAAAAACCACCGCTACCAGCGGTGGTTTGTT
TGCCGGATCAAGAGCTACCAACTCTTTTTCCGAAGGTAACTGGCTTCAGCAGAGCGCAGATACCAAATACTGTCCTTCTAGTGTA
GCCGTAGTTAGGCCACCACTTCAAGAACTCTGTAGCACCGCCTACATACCTCGCTCTGCTAATCCTGTTACCAGTGGCTGCTGCC
AGTGGCGATAAGTCGTGTCTTACCGGGTTGGACTCAAGACGATAGTTACCGGATAAGGCGCAGCGGTCGGGCTGAACGGGGGGTT
CGTGCACACAGCCCAGCTTGGAGCGAACGACCTACACCGAACTGAGATACCTACAGCGTGAGCTATGAGAAAGCGCCACGCTTCC
CGAAGGGAGAAAGGCGGACAGGTATCCGGTAAGCGGCAGGGTCGGAACAGGAGAGCGCACGAGGGAGCTTCCAGGGGGAAACGCC
TGGTATCTTTATAGTCCTGTCGGGTTTCGCCACCTCTGACTTGAGCGTCGATTTTTGTGATGCTCGTCAGGGGGGCGGAGCCTAT
GGAAAAACGCCAGCAACGCGGCCTTTTTACGGTTCCTGGCCTTTTGCTGGCCTTTTGCTCACATGT

TABLE 5
Components of Construct Sequence (SEQ ID NO: 100)

| Components | Position in construct |
|---|---|
| 5′ITR | 12-130 |
| Cloning site | 131-147 |
| RBP7 promoter | 148-1244 |
| hGJB2 minimal promoter | 1245-1372 |
| Cloning site | 1373-1381 |
| Synthetic barcode | 1382-1389 |
| 5′UTR | 1390-1751 |
| GJB2 (exon2) | 1763-2440 |
| 3xFLAG | 2453-2518 |
| 3′UTR (exon2) | 2522-3928 |
| bGHpA | 3950-4174 |
| Cloning site | 4175-4208 |
| 3′ITR | 4209-4338 |

Exemplary Construct sequence
(SEQ ID NO: 103)
CCTGCAGGCAGCTGCGCGCTCGCTCGCTCACTGAGGCCGCCCGGGCGTCGGGCGACCTTTGGTCGCCCGGCCTCAGTGAGCGA
GCGAGCGCGCAGAGAGGGAGTGGCCAACTCCATCACTAGGGGTTCCTGCGGCCGCACGCGTGGTAAATAGCTTCCAACGTTTC
CACCCCACCAGCCCTTGCACCACTCCCTGTACTGGCCCTGAGCTTTCTAGTCTTGACTGAAAAGCGGGGAGGCAATGTGGTCT
CTCCTGGTGCACTGTCCCGAGGAAGGCCTGCTCCGCTTCCCCGGAGGAGTCTTCAAAGGATGGAGGTAATTAATAAAAACAAC
CCCTGTACCTCCTCTAAGTGGTCATTAATTAATAAAGAACCTCCAGGCTCCTATAGGAGAGGTCTGTGCACCCCGCGGGCTAT
GAGAAGGCTGGATCACCCAGAAAGACTGAGGATGTGTCCTGGCAAAAACACAGCCTGCCCCTCACACTGCTCCCCACGGGTGC
ACTAGGGAGGAAGAGTTCCCTCGAGGGCCTGAGCAGGCGCCCCACACCTGCACCCGTGCAGAGGGGGCTGGGCCCGCCCTCTG
CGCTCCCGAGGGAGAGCCCTACCCCCTGCATCCCCGGTACCCCGTTCCCTCCAAGGGCCGGAAAGAGGGCCCCGCGCACTGTG
CACTTCTTAGGGGTCCCCCACCCTGCGCCCCCGCCACGGGAAAAAGGTCCCCGCTCTGCGCATCCGGCCCCGGAGGGACAGCC
CCGGTCCTGCACTCCTTGCTCCTCAGGGGGACGGTCCGCGCCCAGCGGCTAGTGCGCCCCGGGTAGGTGGGGGCGGGGGGCTC
GTCGAGTGACAGCGCTCGCCTCCCGCAGCCCGCCCGAGCCGCGTCAGGGCAGAAGCTCTGAGGACCCAGAGGCCGGGCGCGCT
CCGCCCGCGGCGCCGCCCCCTCCGTAACTTTCCCAGTCTCCGAGGGAAGAGGCGGGGTGTGGGGTGCGGTTAAAAGGCGCCAC
GGCGGGAGACAGGTCTCACCGGTTCGTGGGTGTTGCGGCCCCGCAGCGCCCGCGCGCTCCTCTCCCCGACTCGGAGCCCCTCG
GCGGCGCCCGGCCCAGGACCCGCCTAGGAGCGCAGGAGCCCCAGCGCAGAGACCCCAACGCCGAGACCCCCGCCCCGGCCCCG
CCGCGCTTCCTCCCGACGCAGTTTAGGACCCTTGTTCGCGAAGAGGTGGTGTGCGGCTGAGACCCGCGTCCTCAGGACGGTTC
CATCAGTGCCTCGATCCTGCCCCACTGGAGGAGGAAGGCAGCCCGAACAGCGCTCACCTAACTAACAGCTGCTGAGAGCTGGG
TTCCGTGGCCATGCACCTGGGACTGCCTTGAGAAGCGTGAGCAAACCGCCCAGAGTAGAAGCGCTAGCCACCATGGATTGGGG
CACGCTGCAGACGATCCTGGGGGGTGTGAACAAACACTCCACCAGCATTGGAAAGATCTGGCTCACCGTCCTCTTCATTTTTC
GCATTATGATCCTCGTTGTGGCTGCAAAGGAGGTGTGGGGAGATGAGCAGGCCGACTTTGTCTGCAACACCCTGCAGCCAGGC
TGCAAGAACGTGTGCTACGATCACTACTTCCCCATCTCCCACATCCGGCTATGGGCCCTGCAGCTGATCTTCGTGTCCACGCC
AGCGCTCCTAGTGGCCATGCACGTGGCCTACCGGAGACATGAGAAGAAGAGGAAGTTCATCAAGGGGGAGATAAAGAGTGAAT
TTAAGGACATCGAGGAGATCAAAACCCAGAAGGTCCGCATCGAAGGCTCCCTGTGGTGGACCTACACAAGCAGCATCTTCTTC
CGGGTCATCTTCGAAGCCGCCTTCATGTACGTCTTCTATGTCATGTACGACGGCTTCTCCATGCAGCGGCTGGTGAAGTGCAA
CGCCTGGCCTTGTCCCAACACTGTGGACTGCTTTGTGTCCCGGCCCACGGAGAAGACTGTCTTCACAGTGTTCATGATTGCAG
TGTCTGGAATTTGCATCCTGCTGAATGTCACTGAATTGTGTTATTTGCTAATTAGATATTGTTCTGGGAAGTCAAAAAAGCCA
GTTGGATCCCGGGCTGACTACAAAGACCATGACGGTGATTATAAAGATCATGACATCGACTACAAGGATGACGATGACAAGTA
AGAAATAGACAGCATGAGAGGGATGAGGCAACCCGTGCTCAGCTGTCAAGGCTCAGTCGCTAGCATTTCCCAACACAAAGATT
CTGACCTTAAATGCAACCATTTGAAACCCCTGTAGGCCTCAGGTGAAACTCCAGATGCCACAATGGAGCTCTGCTCCCCTAAA
GCCTCAAAACAAAGGCCTAATTCTATGCCTGTCTTAATTTTCTTTCACTTAAGTTAGTTCCACTGAGACCCCAGGCTGTTAGG
GGTTATTGGTGTAAGGTACTTTCATATTTTAAACAGAGGATATCGGCATTTGTTTCTTTCTCTGAGGACAAGAGAAAAAAGCC
AGGTTCCACAGAGGACACAGAGAAGGTTTGGGTGTCCTCCTGGGGTTCTTTTTGCCAACTTTCCCCACGTTAAAGGTGAACAT
TGGTTCTTTCATTTGCTTTGGAAGTTTTAATCTCTAACAGTGGACAAAGTTACCAGTGCCTTAAACTCTGTTACACTTTTTGG
AAGTGAAAACTTTGTAGTATGATAGGTTATTTTGATGTAAAGATGTTCTGGATACCATTATATGTTCCCCCTGTTTCAGAGGC
TCAGATTGTAATATGTAAATGGTATGTCATTCGCTACTATGATTTAATTTGAAATATGGTCTTTTGGTTATGAATACTTTGCA
GCACAGCTGAGAGGCTGTCTGTTGTATTCATTGTGGTCATAGCACCTAACAACATTGTAGCCTCAATCGAGTGAGACAGACTA
GAAGTTCCTAGTGATGGCTTATGATAGCAAATGGCCTCATGTCAAATATTTAGATGTAATTTTGTGTAAGAAATACAGACTGG
ATGTACCACCAACTACTACCTGTAATGACAGGCCTGTCCAACACATCTCCCTTTTCCATGACTGTGGTAGCCAGCATCGGAAA
GAACGCTGATTTAAAGAGGTCGCTTGGGAATTTTATTGACACAGTACCATTTAATGGGGAGGACAAAATGGGGCAGGGGAGGG
AGAAGTTTCTGTCGTTAAAAACAGATTTGGAAAGACTGGACTCTAAAGTCTGTTGATTAAAGATGAGCTTTGTCTACTTCAAA
AGTTTGTTTGCTTACCCCTTCAGCCTCCAATTTTTTAAGTGAAAATATAGCTAATAACATGTGAAAAGAATAGAAGCTAAGGT
TTAGATAAATATTGAGCAGATCTATAGGAAGATTGAACCTGAATATTGCCATTATGCTTGACATGGTTTCCAAAAAATGGTAC
TCCACATATTTCAGTGAGGGTAAGTATTTTCCTGTTGTCAAGAATAGCATTGTAAAAGCATTTTGTAATAATAAAGAATAGCT
TTAATGATATGCTTGTAACTAAAATAATTTTGTAATGTATCAAATACATTTAAAACATTAAAATATAATCTCTATAATAAGAG
CTCGCTGATCAGCCTCGACTGTGCCTTCTAGTTGCCAGCCATCTGTTGTTTGCCCCTCCCCCGTGCCTTCCTTGACCCTGGAA
GGTGCCACTCCCACTGTCCTTTCCTAATAAAATGAGGAAATTGCATCGCATTGTCTGAGTAGGTGTCATTCTATTCTGGGGGG
TGGGGTGGGGCAGGACAGCAAGGGGGAGGATTGGGAAGACAATAGCAGGCATGCTGGGGATGCGGTGGGCTCTATGGAAGCTT
GAATTCAGCTGACGTGCCTCGGACCGCTAGGAACCCCTAGTGATGGAGTTGGCCACTCCCTCTCTGCGCGCTCGCTCGCTCAC
TGAGGCCGGGCGACCAAAGGTCGCCCGACGCCCGGGCTTTGCCCGGGCGGCCTCAGTGAGCGAGCGAGCGCGCAGCTGCCTGC
AGGGGCGCCTGATGCGGTATTTTCTCCTTACGCATCTGTGCGGTATTTCACACCGCATACGTCAAAGCAACCATAGTACGCGC
CCTGTAGCGGCGCATTAAGCGCGGCGGGTGTGGTGGTTACGCGCAGCGTGACCGCTACACTTGCCAGCGCCCTAGCGCCCGCT
CCTTTCGCTTTCTTCCCTTCCTTTCTCGCCACGTTCGCCGGCTTTCCCCGTCAAGCTCTAAATCGGGGGCTCCCTTTAGGGTT
CCGATTTAGTGCTTTACGGCACCTCGACCCCAAAAAACTTGATTTGGGTGATGGTTCACGTAGTGGGCCATCGCCCTGATAGA
CGGTTTTTCGCCCTTTGACGTTGGAGTCCACGTTCTTTAATAGTGGACTCTTGTTCCAAACTGGAACAACACTCAACCCTATC
TCGGGCTATTCTTTTGATTTATAAGGGATTTTGCCGATTTCGGCCTATTGGTTAAAAAATGAGCTGATTTAACAAAAATTTAA
CGCGAATTTTAACAAAATATTAACGTTTACAATTTTATGGTGCACTCTCAGTACAATCTGCTCTGATGCCGCATAGTTAAGCC
AGCCCCGACACCCGCCAACACCCGCTGACGCGCCCTGACGGGCTTGTCTGCTCCCGGCATCCGCTTACAGACAAGCTGTGACC
GTCTCCGGGAGCTGCATGTGTCAGAGGTTTTCACCGTCATCACCGAAACGCGCGAGACGAAAGGGCCTCGTGATACGCCTATT
TTTATAGGTTAATGTCATGAACAATAAAACTGTCTGCTTACATAAACAGTAATACAAGGGGTGTTATGAGCCATATTCAACGG
GAAACGTCGAGGCCGCGATTAAATTCCAACATGGATGCTGATTTATATGGGTATAAATGGGCTCGCGATAATGTCGGGCAATC
AGGTGCGACAATCTATCGCTTGTATGGGAAGCCCGATGCGCCAGAGTTGTTTCTGAAACATGGCAAAGGTAGCGTTGCCAATG
ATGTTACAGATGAGATGGTCAGACTAAACTGGCTGACGGAATTTATGCCTCTTCCGACCATCAAGCATTTTATCCGTACTCCT
GATGATGCATGGTTACTCACCACTGCGATCCCCGGAAAAACAGCATTCCAGGTATTAGAAGAATATCCTGATTCAGGTGAAAA
TATTGTTGATGCGCTGGCAGTGTTCCTGCGCCGGTTGCATTCGATTCCTGTTTGTAATTGTCCTTTTAACAGCGATCGCGTAT
TTCGTCTCGCTCAGGCGCAATCACGAATGAATAACGGTTTGGTTGATGCGAGTGATTTTGATGACGAGCGTAATGGCTGGCCT
GTTGAACAAGTCTGGAAAGAAATGCATAAACTTTTGCCATTCTCACCGGATTCAGTCGTCACTCATGGTGATTTCTCACTTGA
TAACCTTATTTTTGACGAGGGGAAATTAATAGGTTGTATTGATGTTGGACGAGTCGGAATCGCAGACCGATACCAGGATCTTG
CCATCCTATGGAACTGCCTCGGTGAGTTTTCTCCTTCATTACAGAAACGGCTTTTTCAAAAATATGGTATTGATAATCCTGAT
ATGAATAAATTGCAGTTTCATTTGATGCTCGATGAGTTTTTCTAATCTCATGACCAAAATCCCTTAACGTGAGTTTTCGTTCC
ACTGAGCGTCAGACCCCGTAGAAAAGATCAAAGGATCTTCTTGAGATCCTTTTTTTCTGCGCGTAATCTGCTGCTTGCAAACA
AAAAAACCACCGCTACCAGCGGTGGTTTGTTTGCCGGATCAAGAGCTACCAACTCTTTTTCCGAAGGTAACTGGCTTCAGCAG
AGCGCAGATACCAAATACTGTCCTTCTAGTGTAGCCGTAGTTAGGCCACCACTTCAAGAACTCTGTAGCACCGCCTACATACC
TCGCTCTGCTAATCCTGTTACCAGTGGCTGCTGCCAGTGGCGATAAGTCGTGTCTTACCGGGTTGGACTCAAGACGATAGTTA
CCGGATAAGGCGCAGCGGTCGGGCTGAACGGGGGGTTCGTGCACACAGCCCAGCTTGGAGCGAACGACCTACACCGAACTGAG
ATACCTACAGCGTGAGCTATGAGAAAGCGCCACGCTTCCCGAAGGGAGAAAGGCGGACAGGTATCCGGTAAGCGGCAGGGTCG
GAACAGGAGAGCGCACGAGGGAGCTTCCAGGGGGAAACGCCTGGTATCTTTATAGTCCTGTCGGGTTTCGCCACCTCTGACTT
GAGCGTCGATTTTTGTGATGCTCGTCAGGGGGGCGGAGCCTATGGAAAAACGCCAGCAACGCGGCCTTTTTACGGTTCCTGGC
CTTTTGCTGGCCTTTTGCTCACATGT

TABLE 6
Components of Construct Sequence (SEQ ID NO: 103)

| Components | Position in construct |
|---|---|
| 5′ITR | 12-130 |
| Cloning site | 131-147 |
| GJB66 promoter | 148-882 |
| hGJB2 minimal promoter | 883-1010 |
| Cloning site | 1011-1019 |
| Synthetic barcode | 1020-1027 |
| 5′UTR | 1028-1389 |
| GJB2 (exon2) | 1401-2078 |
| 3xFLAG | 2091-2156 |
| 3′UTR (exon2) | 2160-3566 |
| bGHpA | 3588-3812 |
| Cloning site | 3813-3846 |
| 3′ITR | 3847-3976 |

Exemplary Construct sequence
(SEQ ID NO: 106)
CCTGCAGGCAGCTGCGCGCTCGCTCGCTCACTGAGGCCGCCCGGGCGTCGGGCGACCTTTGGTCGCCCGGCCTCAGTG
AGCGAGCGAGCGCGCAGAGAGGGAGTGGCCAACTCCATCACTAGGGGTTCCTGCGGCCGCACGCGTGGTTGTACAGGA
GATAGTCAGGGAATTAGTAATTTTCAAAGAGGTGACTTTGAATTCAAACTTAAATATCATCTTCAGCTGAAACAAAGA
AGGGGTGCAGTTATGAGGAAGTGACCAGGTAAAGCATGGCAAACAAAGGTAAAGTTTGTTATGCGTATTTAAGTCAGA
GCCCTCTCCATTGATAAGAGTTTCCAGTAATTTAGTGCCATCCTTTTCTTGCTATAGAGTTCTCGTCTCTATCTGAGC
ACGCAAAAATAACATGCTTTCTTGCTTTCTTGAAGTTGGGCATGGCCATTGACTTGCCTTAGCCCATATTTTTCTGTG
AAGTGGTCTTCAAAAACCTATATTTCTGCCATAGAGTCACTTACTTAACCTGCCCTATTTAAAGGGGCTAATGCCTGA
TAGAATGTCGCTGCATAACTCCATCTGTGTGTGGTCCCTGCATCCATGACAACCAAAACCCAGATGCAGAAATTGTTC
CTAATCACATAGATTACCCTAGAAACCGGAAGGGCCTTGAAGTCAAAAGCATTCAGAGAACATGCTGAACAAATTGAA
TTTGCAGTTTATCTGGCCAGGGAGGATGGAGAGGGGATGGGCACTTGGTCTGAGTATTTTTTGTTTCTCATTCCAACA
GAAATTACTAGATTTACCAAAAAATCTACAAGTGGTAGTGTGATAGAGTCAGGCAGAGGAATTGACCATAGATAAGGT
GCTCAGGACTCCTAGAGTCAGCTTCTGGTATGTGAGAAAGAAGTGAGAACAGAGCCCATGGCATATGAAGAAGATATT
ACAGAAAAAAGAAAGCTGCCTTCCACGCAAATCATTTCTTTACAAAGGCTTGTTAACTCCTGCAGTGCCAAGAAGCTG
AATGCAGCGGCAGACATCCTGGTTCGGGCCCCAGGAAGCTCAGCCGGGTTTAATGTGGATGAGGGTTTAATGATGTAC
ACGCAGAAGTGTTTTGACAAATGAAGAAGGTCCTCATTCTTGGAACATGTGCCGGTTCTCCGAGGGAACTCCTAAAAG
GCTGTAAGCTCATGTAGGAAAAGCTGAGCTAGATTCCTAAGGGCAGAGATGTGCTCACATTTCTTTGCATCCCTAGTT
CCCAGCACAGTGCAAGGCGCTGCAAACATTTGCTGAACCCAGGGTCTCGTGTCTTGACTGTCCAGCAGAGGCCGCTCT
GGGCCGGGGCTCTCGGGACCTGAGGGCTGAGAGAAGGAAGGCCAGGGGGTGGCCCAGTCATCGCCGCGGGGCCCGGGT
GGGAGGGGTTTGGCAGCGGCAGGCGCGGCGGCGGCGGCGGAGGCGGAGGCGGCCCCGGGAAGCTCTGAGGACCCAGAG
GCCGGGCGCGCTCCGCCCGCGGCGCCGCCCCCTCCGTAACTTTCCCAGTCTCCGAGGGAAGAGGCGGGGTGTGGGGTG
CGGTTAAAAGGCGCCACGGCGGGAGACAGGTCTCACCGGTGCAAACTGGTTGCGGCCCCGCAGCGCCCGCGCGCTCCT
CTCCCCGACTCGGAGCCCCTCGGCGGCGCCCGGCCCAGGACCCGCCTAGGAGCGCAGGAGCCCCAGCGCAGAGACCCC
AACGCCGAGACCCCCGCCCCGGCCCCGCCGCGCTTCCTCCCGACGCAGTTTAGGACCCTTGTTCGCGAAGAGGTGGTG
TGCGGCTGAGACCCGCGTCCTCAGGACGGTTCCATCAGTGCCTCGATCCTGCCCCACTGGAGGAGGAAGGCAGCCCGA
ACAGCGCTCACCTAACTAACAGCTGCTGAGAGCTGGGTTCCGTGGCCATGCACCTGGGACTGCCTTGAGAAGCGTGAG
CAAACCGCCCAGAGTAGAAGCGCTAGCCACCATGGATTGGGGCACGCTGCAGACGATCCTGGGGGGTGTGAACAAACA
CTCCACCAGCATTGGAAAGATCTGGCTCACCGTCCTCTTCATTTTTCGCATTATGATCCTCGTTGTGGCTGCAAAGGA
GGTGTGGGGAGATGAGCAGGCCGACTTTGTCTGCAACACCCTGCAGCCAGGCTGCAAGAACGTGTGCTACGATCACTA
CTTCCCCATCTCCCACATCCGGCTATGGGCCCTGCAGCTGATCTTCGTGTCCACGCCAGCGCTCCTAGTGGCCATGCA
CGTGGCCTACCGGAGACATGAGAAGAAGAGGAAGTTCATCAAGGGGGAGATAAAGAGTGAATTTAAGGACATCGAGGA
GATCAAAACCCAGAAGGTCCGCATCGAAGGCTCCCTGTGGTGGACCTACACAAGCAGCATCTTCTTCCGGGTCATCTT
CGAAGCCGCCTTCATGTACGTCTTCTATGTCATGTACGACGGCTTCTCCATGCAGCGGCTGGTGAAGTGCAACGCCTG
GCCTTGTCCCAACACTGTGGACTGCTTTGTGTCCCGGCCCACGGAGAAGACTGTCTTCACAGTGTTCATGATTGCAGT
GTCTGGAATTTGCATCCTGCTGAATGTCACTGAATTGTGTTATTTGCTAATTAGATATTGTTCTGGGAAGTCAAAAAA
GCCAGTTGGATCCCGGGCTGACTACAAAGACCATGACGGTGATTATAAAGATCATGACATCGACTACAAGGATGACGA
TGACAAGTAAGAAATAGACAGCATGAGAGGGATGAGGCAACCCGTGCTCAGCTGTCAAGGCTCAGTCGCTAGCATTTC
CCAACACAAAGATTCTGACCTTAAATGCAACCATTTGAAACCCCTGTAGGCCTCAGGTGAAACTCCAGATGCCACAAT
GGAGCTCTGCTCCCCTAAAGCCTCAAAACAAAGGCCTAATTCTATGCCTGTCTTAATTTTCTTTCACTTAAGTTAGTT
CCACTGAGACCCCAGGCTGTTAGGGGTTATTGGTGTAAGGTACTTTCATATTTTAAACAGAGGATATCGGCATTTGTT
TCTTTCTCTGAGGACAAGAGAAAAAAGCCAGGTTCCACAGAGGACACAGAGAAGGTTTGGGTGTCCTCCTGGGGTTCT
TTTTGCCAACTTTCCCCACGTTAAAGGTGAACATTGGTTCTTTCATTTGCTTTGGAAGTTTTAATCTCTAACAGTGGA
CAAAGTTACCAGTGCCTTAAACTCTGTTACACTTTTTGGAAGTGAAAACTTTGTAGTATGATAGGTTATTTTGATGTA
AAGATGTTCTGGATACCATTATATGTTCCCCCTGTTTCAGAGGCTCAGATTGTAATATGTAAATGGTATGTCATTCGC
TACTATGATTTAATTTGAAATATGGTCTTTTGGTTATGAATACTTTGCAGCACAGCTGAGAGGCTGTCTGTTGTATTC
ATTGTGGTCATAGCACCTAACAACATTGTAGCCTCAATCGAGTGAGACAGACTAGAAGTTCCTAGTGATGGCTTATGA
TAGCAAATGGCCTCATGTCAAATATTTAGATGTAATTTTGTGTAAGAAATACAGACTGGATGTACCACCAACTACTAC
CTGTAATGACAGGCCTGTCCAACACATCTCCCTTTTCCATGACTGTGGTAGCCAGCATCGGAAAGAACGCTGATTTAA
AGAGGTCGCTTGGGAATTTTATTGACACAGTACCATTTAATGGGGAGGACAAAATGGGGCAGGGGAGGGAGAAGTTTC
TGTCGTTAAAAACAGATTTGGAAAGACTGGACTCTAAAGTCTGTTGATTAAAGATGAGCTTTGTCTACTTCAAAAGTT
TGTTTGCTTACCCCTTCAGCCTCCAATTTTTTAAGTGAAAATATAGCTAATAACATGTGAAAAGAATAGAAGCTAAGG
TTTAGATAAATATTGAGCAGATCTATAGGAAGATTGAACCTGAATATTGCCATTATGCTTGACATGGTTTCCAAAAAA
TGGTACTCCACATATTTCAGTGAGGGTAAGTATTTTCCTGTTGTCAAGAATAGCATTGTAAAAGCATTTTGTAATAAT
AAAGAATAGCTTTAATGATATGCTTGTAACTAAAATAATTTTGTAATGTATCAAATACATTTAAAACATTAAAATATA
ATCTCTATAATAAGAGCTCGCTGATCAGCCTCGACTGTGCCTTCTAGTTGCCAGCCATCTGTTGTTTGCCCCTCCCCC
GTGCCTTCCTTGACCCTGGAAGGTGCCACTCCCACTGTCCTTTCCTAATAAAATGAGGAAATTGCATCGCATTGTCTG
AGTAGGTGTCATTCTATTCTGGGGGGTGGGGTGGGGCAGGACAGCAAGGGGGAGGATTGGGAAGACAATAGCAGGCAT
GCTGGGGATGCGGTGGGCTCTATGGAAGCTTGAATTCAGCTGACGTGCCTCGGACCGCTAGGAACCCCTAGTGATGGA
GTTGGCCACTCCCTCTCTGCGCGCTCGCTCGCTCACTGAGGCCGGGCGACCAAAGGTCGCCCGACGCCCGGGCTTTGC
CCGGGCGGCCTCAGTGAGCGAGCGAGCGCGCAGCTGCCTGCAGGGGCGCCTGATGCGGTATTTTCTCCTTACGCATCT
GTGCGGTATTTCACACCGCATACGTCAAAGCAACCATAGTACGCGCCCTGTAGCGGCGCATTAAGCGCGGCGGGTGTG
GTGGTTACGCGCAGCGTGACCGCTACACTTGCCAGCGCCCTAGCGCCCGCTCCTTTCGCTTTCTTCCCTTCCTTTCTC
GCCACGTTCGCCGGCTTTCCCCGTCAAGCTCTAAATCGGGGGCTCCCTTTAGGGTTCCGATTTAGTGCTTTACGGCAC
CTCGACCCCAAAAAACTTGATTTGGGTGATGGTTCACGTAGTGGGCCATCGCCCTGATAGACGGTTTTTCGCCCTTTG
ACGTTGGAGTCCACGTTCTTTAATAGTGGACTCTTGTTCCAAACTGGAACAACACTCAACCCTATCTCGGGCTATTCT
TTTGATTTATAAGGGATTTTGCCGATTTCGGCCTATTGGTTAAAAAATGAGCTGATTTAACAAAAATTTAACGCGAAT
TTTAACAAAATATTAACGTTTACAATTTTATGGTGCACTCTCAGTACAATCTGCTCTGATGCCGCATAGTTAAGCCAG
CCCCGACACCCGCCAACACCCGCTGACGCGCCCTGACGGGCTTGTCTGCTCCCGGCATCCGCTTACAGACAAGCTGTG
ACCGTCTCCGGGAGCTGCATGTGTCAGAGGTTTTCACCGTCATCACCGAAACGCGCGAGACGAAAGGGCCTCGTGATA
CGCCTATTTTTATAGGTTAATGTCATGAACAATAAAACTGTCTGCTTACATAAACAGTAATACAAGGGGTGTTATGAG
CCATATTCAACGGGAAACGTCGAGGCCGCGATTAAATTCCAACATGGATGCTGATTTATATGGGTATAAATGGGCTCG
CGATAATGTCGGGCAATCAGGTGCGACAATCTATCGCTTGTATGGGAAGCCCGATGCGCCAGAGTTGTTTCTGAAACA
TGGCAAAGGTAGCGTTGCCAATGATGTTACAGATGAGATGGTCAGACTAAACTGGCTGACGGAATTTATGCCTCTTCC
GACCATCAAGCATTTTATCCGTACTCCTGATGATGCATGGTTACTCACCACTGCGATCCCCGGAAAAACAGCATTCCA
GGTATTAGAAGAATATCCTGATTCAGGTGAAAATATTGTTGATGCGCTGGCAGTGTTCCTGCGCCGGTTGCATTCGAT
TCCTGTTTGTAATTGTCCTTTTAACAGCGATCGCGTATTTCGTCTCGCTCAGGCGCAATCACGAATGAATAACGGTTT
GGTTGATGCGAGTGATTTTGATGACGAGCGTAATGGCTGGCCTGTTGAACAAGTCTGGAAAGAAATGCATAAACTTTT
GCCATTCTCACCGGATTCAGTCGTCACTCATGGTGATTTCTCACTTGATAACCTTATTTTTGACGAGGGGAAATTAAT
AGGTTGTATTGATGTTGGACGAGTCGGAATCGCAGACCGATACCAGGATCTTGCCATCCTATGGAACTGCCTCGGTGA
GTTTTCTCCTTCATTACAGAAACGGCTTTTTCAAAAATATGGTATTGATAATCCTGATATGAATAAATTGCAGTTTCA
TTTGATGCTCGATGAGTTTTTCTAATCTCATGACCAAAATCCCTTAACGTGAGTTTTCGTTCCACTGAGCGTCAGACC
CCGTAGAAAAGATCAAAGGATCTTCTTGAGATCCTTTTTTTCTGCGCGTAATCTGCTGCTTGCAAACAAAAAAACCAC
CGCTACCAGCGGTGGTTTGTTTGCCGGATCAAGAGCTACCAACTCTTTTTCCGAAGGTAACTGGCTTCAGCAGAGCGC
AGATACCAAATACTGTCCTTCTAGTGTAGCCGTAGTTAGGCCACCACTTCAAGAACTCTGTAGCACCGCCTACATACC
TCGCTCTGCTAATCCTGTTACCAGTGGCTGCTGCCAGTGGCGATAAGTCGTGTCTTACCGGGTTGGACTCAAGACGAT
AGTTACCGGATAAGGCGCAGCGGTCGGGCTGAACGGGGGGTTCGTGCACACAGCCCAGCTTGGAGCGAACGACCTACA
CCGAACTGAGATACCTACAGCGTGAGCTATGAGAAAGCGCCACGCTTCCCGAAGGGAGAAAGGCGGACAGGTATCCGG
TAAGCGGCAGGGTCGGAACAGGAGAGCGCACGAGGGAGCTTCCAGGGGGAAACGCCTGGTATCTTTATAGTCCTGTCG
GGTTTCGCCACCTCTGACTTGAGCGTCGATTTTTGTGATGCTCGTCAGGGGGGCGGAGCCTATGGAAAAACGCCAGCA
ACGCGGCCTTTTTACGGTTCCTGGCCTTTTGCTGGCCTTTTGCTCACATGT

TABLE 7
Components of Construct Sequence (SEQ ID NO: 106)

| Components | Position in construct |
|---|---|
| 5′ITR | 12-130 |
| Cloning site | 131-147 |
| PARM1 promoter | 148-1463 |
| hGJB2 minimal promoter | 1464-1591 |
| Cloning site | 1592-1600 |
| Synthetic barcode | 1601-1608 |
| 5′UTR | 1609-1970 |
| GJB2 (exon2) | 1982-2659 |
| 3xFLAG | 2672-2737 |
| 3′UTR (exon2) | 2741-4147 |
| bGHpA | 4169-4393 |
| Cloning site | 4394-4427 |
| 3′ITR | 4428-4557 |

Multiple AAV Construct Compositions

The present disclosure recognizes that some coding sequences encoding a protein (e.g., connexin 26 protein) may be delivered by dividing the coding sequence into multiple portions, which are each included in a different construct. In some embodiments, provided herein are compositions or systems comprising at least two different constructs, (e.g., two, three, four, five, or six). In some embodiments, each of the at least two different constructs includes a coding sequence that encodes a different portion of a coding region (e.g., encoding a target protein (e.g., an inner ear target protein, e.g., a connexin 26 protein)), each of the encoded portions being at least 10 amino acids (e.g., at least about 10 amino acids, at least about 20 amino acids, at least about 30 amino acids, at least about 60 amino acids, at least about 70 amino acids, at least about 80 amino acids, at least about 90 amino acids, at least about 100 amino acids, at least about 110 amino acids, at least about 120 amino acids, at least about 130 amino acids, at least about 140 amino acids, at least about 150 amino acids, at least about 160 amino acids, at least about 170 amino acids, at least about 180 amino acids, at least about 190 amino acids, at least about 200 amino acids, at least about 210 amino acids, at least about 220 amino acids, at least about 230 amino acids, at least about 240 amino acids, at least about 250 amino acids, or at least about 260 amino acids) where the amino acid sequence of each of the encoded portions may optionally partially overlap with the amino acid sequence of a different one of the encoded portions; no single construct of the at least two different constructs encodes the active target protein; and, when introduced into a subject cell (e.g., an animal cell, e.g., a primate cell, e.g., a human cell), the at least two different constructs undergo homologous recombination with each other, where the recombined nucleic acid encodes an active target protein (e.g., a gene product encoded by a GJB2 gene or a characteristic portion thereof). In some embodiments, one of the nucleic acid constructs can include a coding sequence that encodes a portion of a target protein (e.g., an inner ear target protein, e.g., a connexin 26 protein), where the encoded portion is at most about 260 amino acids (e.g., at most about 10 amino acids, at most about 20 amino acids, at most about 30 amino acids, at most about 60 amino acids, at most about 70 amino acids, at most about 80 amino acids, at most about 90 amino acids, at most about 100 amino acids, at most about 110 amino acids, at most about 120 amino acids, at most about 130 amino acids, at most about 140 amino acids, at most about 150 amino acids, at most about 160 amino acids, at most about 170 amino acids, at most about 180 amino acids, at most about 190 amino acids, at most about 200 amino acids, at most about 210 amino acids, at most about 220 amino acids, at most about 230 amino acids, at most about 240 amino acids, at most about 250 amino acids, or at most about 260 amino acids).

In some embodiments, at least one of the constructs includes a nucleotide sequence spanning two neighboring exons of target genomic DNA (e.g., an inner ear target genomic DNA, e.g., GJB2 genomic DNA), and lacks the intronic sequence that naturally occurs between the two neighboring exons.

In some embodiments, an amino acid sequence of an encoded portion of each of the constructs does not overlap, even in part, with an amino acid sequence of a different one of the encoded portions. In some embodiments, an amino acid sequence of an encoded portion of a construct partially overlaps with an amino acid sequence of an encoded portion of a different construct. In some embodiments, an amino acid sequence of an encoded portion of each construct partially overlaps with an amino acid sequence of an encoded portion of at least one different construct. In some embodiments, an overlapping amino acid sequence is between about 10 amino acid residues to about 260 amino acids, or any of the subranges of this range (e.g., about 10 amino acids, about 20 amino acids, about 30 amino acids, about 60 amino acids, about 70 amino acids, about 80 amino acids, about 90 amino acids, about 100 amino acids, about 110 amino acids, about 120 amino acids, about 130 amino acids, about 140 amino acids, about 150 amino acids, about 160 amino acids, about 170 amino acids, about 180 amino acids, about 190 amino acids, about 200 amino acids, about 210 amino acids, about 220 amino acids, about 230 amino acids, about 240 amino acids, about 250 amino acids, or about 260 amino acids) in length.

In some examples, a desired gene product (e.g., a therapeutic gene product) is encoded by at least two different constructs. In some embodiments, each of at least two different constructs includes a different segment of an intron, where the intron includes a nucleotide sequence of an intron that is present in a target genomic DNA (e.g., an inner ear cell target genomic DNA (e.g., GJB2 genomic DNA) (e.g., any of the exemplary introns in SEQ ID NO: 5 described herein). In some embodiments, different intron segments overlap. In some embodiments, different intron segments overlap in sequence by at most about 3,000 nucleotides (e.g., at most about 100 nucleotides, at most about 200 nucleotides, at most about 300 nucleotides, at most about 600 nucleotides, at most about 700 nucleotides, at most about 800 nucleotides, at most about 900 nucleotides, at most about 1,000 nucleotides, at most about 1,100 nucleotides, at most about 1,200 nucleotides, at most about 1,300 nucleotides, at most about 1,400 nucleotides, at most about 1,500 nucleotides, at most about 1,600 nucleotides, at most about 1,700 nucleotides, at most about 1,800 nucleotides, at most about 1,900 nucleotides, at most about 2,000 nucleotides, at most about 2,100 nucleotides, at most about 2,200 nucleotides, at most about 2,300 nucleotides, at most about 2,400 nucleotides, at most about 2,500 nucleotides, at most about 2,600 nucleotides, at most about 2,700 nucleotides, at most about 2,800 nucleotides, at most about 2,900 nucleotides, or at most about 3,000 nucleotides) in length. In some embodiments, the overlapping nucleotide sequence in any two of the different constructs can include part or all of one or more exons of a target gene (e.g., an inner ear cell target gene (e.g., a GJB2 gene) (e.g., any one or more of the exemplary exons in SEQ ID NO: 5 described herein).

In some embodiments, a composition or system is or comprises two, three, four, or five different constructs. In compositions where the number of different constructs in the composition is two, the first of the two different constructs can include a coding sequence that encodes an N-terminal portion of a protein (e.g., connexin 26 protein), which may be referred to as a lead portion, a first construct, or a 5′ portion (e.g., an N-terminal portion of an inner ear cell protein, e.g., an N-terminal portion of a connexin 26 protein). In some examples, an N-terminal portion of the target gene is at least about 10 amino acids (e.g., at least about 10 amino acids, at least about 20 amino acids, at least about 30 amino acids, at least about 60 amino acids, at least about 70 amino acids, at least about 80 amino acids, at least about 90 amino acids, at least about 100 amino acids, at least about 110 amino acids, at least about 120 amino acids, at least about 130 amino acids, at least about 140 amino acids, at least about 150 amino acids, at least about 160 amino acids, at least about 170 amino acids, at least about 180 amino acids, at least about 190 amino acids, at least about 200 amino acids, at least about 210 amino acids, at least about 220 amino acids, at least about 230 amino acids, at least about 240 amino acids, at least about 250 amino acids, or at least about 260 amino acids) in length. In some examples, a first construct includes one or both of a promoter (e.g., any of the promoters described herein or known in the art) and a Kozak sequence (e.g., any of the exemplary Kozak sequences described herein or known in the art). In some examples, a first construct includes a promoter that is an inducible promoter, a constitutive promoter, or a tissue-specific promoter. In some examples, a second of the two different constructs includes a coding sequence that encodes a C-terminal portion of the protein, which may be referred to as a terminal portion, a second construct, or a 3′ portion (e.g., a C-terminal portion of an inner ear cell target protein, e.g., a C-terminal portion of a connexin 26 protein). In some examples, a C-terminal portion of the target protein is at least about 10 amino acids (e.g., at least about 10 amino acids, at least about 20 amino acids, at least about 30 amino acids, at least about 60 amino acids, at least about 70 amino acids, at least about 80 amino acids, at least about 90 amino acids, at least about 100 amino acids, at least about 110 amino acids, at least about 120 amino acids, at least about 130 amino acids, at least about 140 amino acids, at least about 150 amino acids, at least about 160 amino acids, at least about 170 amino acids, at least about 180 amino acids, at least about 190 amino acids, at least about 200 amino acids, at least about 210 amino acids, at least about 220 amino acids, at least about 230 amino acids, at least about 240 amino acids, at least about 250 amino acids, or at least about 260 amino acids) in length. In some examples, a second construct further includes a poly(A) sequence.

In some examples where the number of different constructs in the composition is two, an N-terminal portion encoded by one of the two constructs can include a portion including amino acid position 1 to about amino acid position 260, or any subrange of this range, (e.g., amino acid 1 to at least about amino acid 10, amino acid 1 to at least about amino acid 20, amino acid 1 to at least about amino acid 30, amino acid 1 to at least about amino acid 60, amino acid 1 to at least about amino acid 70, amino acid 1 to at least about amino acid 80, amino acid 1 to at least about amino acid 90, amino acid 1 to at least about amino acid 100, amino acid 1 to at least about amino acid 110, amino acid 1 to at least about amino acid 120, amino acid 1 to at least about amino acid 130, amino acid 1 to at least about amino acid 140, amino acid 1 to at least about amino acid 150, amino acid 1 to at least about amino acid 160, amino acid 1 to at least about amino acid 170, amino acid 1 to at least about amino acid 180, amino acid 1 to at least about amino acid 190, amino acid 1 to at least about amino acid 200, amino acid 1 to at least about amino acid 210, amino acid 1 to at least about amino acid 220, amino acid 1 to at least about amino acid 230, amino acid 1 to at least about amino acid 240, amino acid 1 to at least about amino acid 250, or amino acid 1 to at least about amino acid 260) of an inner ear cell target protein (e.g., SEQ ID NO: 7). In some examples where the number of different constructs in the composition is two, an N-terminal portion of the precursor inner ear cell target protein can include a portion including at most amino acid position 1 to amino acid position 260 or any subrange of this range (e.g., amino acid 1 to at most about amino acid 10, amino acid 1 to at most about amino acid 20, amino acid 1 to at most about amino acid 30, amino acid 1 to at most about amino acid 60, amino acid 1 to at most about amino acid 70, amino acid 1 to at most about amino acid 80, amino acid 1 to at most about amino acid 90, amino acid 1 to at most about amino acid 100, amino acid 1 to at most about amino acid 110, amino acid 1 to at most about amino acid 120, amino acid 1 to at most about amino acid 130, amino acid 1 to at most about amino acid 140, amino acid 1 to at most about amino acid 150, amino acid 1 to at most about amino acid 160, amino acid 1 to at most about amino acid 170, amino acid 1 to at most about amino acid 180, amino acid 1 to at most about amino acid 190, amino acid 1 to at most about amino acid 200, amino acid 1 to at most about amino acid 210, amino acid 1 to at most about amino acid 220, amino acid 1 to at most about amino acid 230, amino acid 1 to at most about amino acid 240, amino acid 1 to at most about amino acid 250, or amino acid 1 to at most about amino acid 260) of an inner ear cell target protein (e.g., SEQ ID NO: 7)

In some examples where the number of different constructs in the composition is two, a C-terminal portion encoded by one of the two constructs can include a portion including the final amino acid (e.g., about amino acid position 260) to about amino acid position 1, or any subrange of this range, (e.g., amino acid 260 to at least about amino acid 10, amino acid 260 to at least about amino acid 20, amino acid 260 to at least about amino acid 30, amino acid 260 to at least about amino acid 60, amino acid 260 to at least about amino acid 70, amino acid 260 to at least about amino acid 80, amino acid 260 to at least about amino acid 90, amino acid 260 to at least about amino acid 100, amino acid 260 to at least about amino acid 110, amino acid 260 to at least about amino acid 120, amino acid 260 to at least about amino acid 130, amino acid 260 to at least about amino acid 140, amino acid 260 to at least about amino acid 150, amino acid 260 to at least about amino acid 160, amino acid 260 to at least about amino acid 170, amino acid 260 to at least about amino acid 180, amino acid 260 to at least about amino acid 190, amino acid 260 to at least about amino acid 200, amino acid 260 to at least about amino acid 210, amino acid 260 to at least about amino acid 220, amino acid 260 to at least about amino acid 230, amino acid 260 to at least about amino acid 240, amino acid 260 to at least about amino acid 250, amino acid 260 to at least about amino acid 260) of an inner ear cell target protein (e.g., SEQ ID NO: 7). In some examples where the number of different constructs in the composition is two, a C-terminal portion of the precursor inner ear cell target protein can include a portion including the final amino acid (e.g., about amino acid position 2600) to at most about amino acid position 1, or any subrange of this range (e.g., amino acid 260 to at most about amino acid 10, amino acid 260 to at most about amino acid 20, amino acid 260 to at most about amino acid 30, amino acid 260 to at most about amino acid 60, amino acid 260 to at most about amino acid 70, amino acid 260 to at most about amino acid 80, amino acid 260 to at most about amino acid 90, amino acid 260 to at most about amino acid 100, amino acid 260 to at most about amino acid 110, amino acid 260 to at most about amino acid 120, amino acid 260 to at most about amino acid 130, amino acid 260 to at most about amino acid 140, amino acid 260 to at most about amino acid 150, amino acid 260 to at most about amino acid 160, amino acid 260 to at most about amino acid 170, amino acid 260 to at most about amino acid 180, amino acid 260 to at most about amino acid 190, amino acid 260 to at most about amino acid 200, amino acid 260 to at most about amino acid 210, amino acid 260 to at most about amino acid 220, amino acid 260 to at most about amino acid 230, amino acid 260 to at most about amino acid 240, amino acid 260 to at most about amino acid 250, amino acid 260 to at most about amino acid 260), or any length sequence there between of an inner ear cell target protein (e.g., SEQ ID NO: 7).

In some embodiments, splice sites are involved in trans-splicing. In some embodiments, a splice donor site (Trapani et al., EMBO Mol. Med. 6(2):194-211, 2014, which is incorporated in its entirety herein by reference) follows the coding sequence in the N-terminal construct. In the C-terminal construct, a splice acceptor site may be subcloned just before the coding sequence for GJB2. In some embodiments, within the coding sequence, a silent mutation can be introduced, generating an additional site for restriction digestion.

In some embodiments, any of the constructs provided herein can be included in a composition suitable for administration to an animal for the amelioration of symptoms associated with syndromic and/or nonsyndromic hearing loss.

Pharmaceutical Compositions

Among other things, the present disclosure provides pharmaceutical compositions. In some embodiments, compositions provided herein are suitable for administration to an animal for the amelioration of symptoms associated with syndromic and/or nonsyndromic hearing loss.

In some embodiments, pharmaceutical compositions of the present disclosure may comprise, e.g., a polynucleotide, e.g., one or more constructs, as described herein. In some embodiments, a pharmaceutical composition may comprise one or more AAV particles, e.g., one or more rAAV construct encapsidated by one or more AAV serotype capsids, as described herein.

In some embodiments, a pharmaceutical composition comprises one or more pharmaceutically or physiologically acceptable carriers, diluents or excipients. As used herein, the term “pharmaceutically acceptable carrier” includes solvents, dispersion media, coatings, antibacterial agents, antifungal agents, and the like that are compatible with pharmaceutical administration. Supplementary active compounds can also be incorporated into any of the compositions described herein. Such compositions may include one or more buffers, such as neutral-buffered saline, phosphate-buffered saline, and the like; one or more carbohydrates, such as glucose, mannose, sucrose, and dextran; mannitol; one or more proteins, polypeptides, or amino acids, such as glycine; one or more antioxidants; one or more chelating agents, such as EDTA or glutathione; and/or one or more preservatives. In some embodiments, formulations are in a dosage forms, such as injectable solutions, injectable gels, drug-release capsules, and the like.

In some embodiments, compositions of the present disclosure are formulated for intravenous administration. In some embodiments compositions of the present disclosure are formulated for intra-cochlear administration. In some embodiments, a therapeutic composition is formulated to comprise a lipid nanoparticle, a polymeric nanoparticle, a mini-circle DNA and/or a CELiD DNA.

In some embodiments, a composition disclosed herein is formulated as a sterile suspension for intracochlear administration. In some embodiments, a composition comprises constructs in an amount of at least IE11, at least 5E11, at least 1E12, at least 5E12, at least 1E13, at least 2E13, at least 3E13, at least 4E13, at least 5E13, at least 6E13, at least 7E13, at least 8E13, at least 9E13, or at least 1E14 vector genomes (vg) per milliliter (mL). In some embodiments, a composition comprises constructs in an amount of at most 1E15, at most 5E14, at most 1E14, at most 5E13, at most 1E13, at most 9E12, at most 8E12, at most 7E12, at most 6E12, at most 5E12, at most 4E12, at most 3E12, at most 2E12, or at most 1E12 vector genomes (vg) per milliliter (mL). In some embodiments, a composition comprises constructs in an amount of 1E12 to 1E13, 5E12 to 5E13, or 1E13 to 2E13 vector genomes (vg) per milliliter (mL).

In some embodiments, a therapeutic composition is formulated to comprise a synthetic perilymph solution. For example, in some embodiments, a synthetic perilymph solution includes 20-200 mM NaCl; 1-5 mM KCl; 0.1-10 mM CaCl₂); 1-10 mM glucose; and 2-50 mM HEPES, with a pH between about 6 and about 9. In some embodiments, a therapeutic composition is formulated to comprise a physiologically suitable solution. For example, in some embodiments, a physiologically suitable solution comprises commercially available 1×PBS with pluronic acid F68, prepared to a final concentration of: 8.10 mM Sodium Phosphate Dibasic, 1.5 mM Monopotassium Phosphate, 2.7 mM Potassium Chloride, 172 mM Sodium Chloride, and 0.001% Pluronic Acid F68). In some embodiments, alternative pluronic acids are utilized. In some embodiments, alternative ion concentrations are utilized.

In some embodiments, any of the pharmaceutical compositions described herein may further comprise one or more agents that promote the entry of a nucleic acid or any of the constructs described herein into a mammalian cell (e.g., a liposome or cationic lipid). In some embodiments, any of the constructs described herein can be formulated using natural and/or synthetic polymers. Non-limiting examples of polymers that may be included in any of the compositions described herein can include, but are not limited to, DYNAMIC POLYCONJUGATE® (Arrowhead Research Corp., Pasadena, Calif.), formulations from Mirus Bio (Madison, Wis.) and Roche Madison (Madison, Wis.), PhaseRX polymer formulations such as, without limitation, SMARTT POLYMER TECHNOLOGY® (PhaseRX, Seattle, Wash.), DMRI/DOPE, poloxamer, VAXFECTIN® adjuvant from Vical (San Diego, Calif.), chitosan, cyclodextrin from Calando Pharmaceuticals (Pasadena, Calif.), dendrimers and poly (lactic-co-glycolic acid) (PLGA) polymers, RONDEL™ (RNAi/Oligonucleotide Nanoparticle Delivery) polymers (Arrowhead Research Corporation, Pasadena, Calif.), and pH responsive co-block polymers, such as, but not limited to, those produced by PhaseRX (Seattle, Wash.). Many of these polymers have demonstrated efficacy in delivering oligonucleotides in vivo into a mammalian cell (see, e.g., deFougerolles, Human Gene Ther. 19:125-132, 2008; Rozema et al., Proc. Natl. Acad. Sci. U.S.A. 104:12982-12887, 2007; Rozema et al., Proc. Natl. Acad. Sci. U.S.A. 104:12982-12887, 2007; Hu-Lieskovan et al., Cancer Res. 65:8984-8982, 2005; Heidel et al., Proc. Natl. Acad. Sci. U.S.A. 104:5715-5721, 2007, each of which is incorporated in its entirety herein by reference).

In some embodiments, a composition includes a pharmaceutically acceptable carrier (e.g., phosphate buffered saline, saline, or bacteriostatic water). Upon formulation, solutions will be administered in a manner compatible with a dosage formulation and in such amount as is therapeutically effective. Formulations are easily administered in a variety of dosage forms such as injectable solutions, injectable gels, drug-release capsules, and the like.

In some embodiments, a composition provided herein can be, e.g., formulated to be compatible with their intended route of administration. A non-limiting example of an intended route of administration is local administration (e.g., intra-cochlear administration). In some embodiments, a provided composition comprises one nucleic acid construct. In some embodiments, a provided composition comprises two or more different constructs. In some embodiments, a composition that include a single nucleic acid construct comprising a coding sequence that encodes a connexin 26 protein and/or a functional characteristic portion thereof. In some embodiments, compositions comprise a single nucleic acid construct comprising a coding sequence that encodes a connexin 26 protein and/or a functional characteristic portion thereof, which, when introduced into a mammalian cell, that coding sequence is integrated into the genome of the mammalian cell. In some embodiments, a composition comprising at least two different constructs, e.g., constructs comprise coding sequences that encode a different portion of a connexin 26 protein, the constructs can be combined to generate a sequence encoding an active connexin 26 protein (e.g., a full-length connexin 26 protein) in a mammalian cell, and thereby treat associated syndromic or nonsyndromic sensorineural hearing loss in a subject in need thereof.

Also provided are kits including any of the compositions described herein. In some embodiments, a kit can include a solid composition (e.g., a lyophilized composition including the at least two different constructs described herein) and a liquid for solubilizing the lyophilized composition. In some embodiments, a kit can include a pre-loaded syringe including any of the compositions described herein.

In some embodiments, the kit includes a vial comprising any of the compositions described herein (e.g., formulated as an aqueous composition, e.g., an aqueous pharmaceutical composition).

In some embodiments, the kits can include instructions for performing any of the methods described herein.

### Genetically Modified Cells

The present disclosure also provides a cell (e.g., an animal cell, e.g., a mammalian cell, e.g., a primate cell, e.g., a human cell) that includes any of the nucleic acids, constructs or compositions described herein. In some embodiments, an animal cell is a human cell (e.g., a human supporting cell or a human hair cell). In other embodiments, an animal cell is a non-human mammal (e.g., Simian cell, Felidae cell, Canidae cell etc.). A person skilled in the art will appreciate that the nucleic acids and constructs described herein can be introduced into any animal cell (e.g., the supporting or hair cells of any animal suitable for veterinary intervention). Non-limiting examples of constructs and methods for introducing constructs into animal cells are described herein.

In some embodiments, an animal cell can be any cell of the inner ear, including hair and/or supporting cells. Non-limiting examples such cells include: Hensen's cells, Deiters' cells, cells of the endolymphatic sac and duct, transitional cells in the saccule, utricle, and ampulla, inner and outer hair cells, spiral ligament cells, spiral ganglion cells, spiral prominence cells, external saccule cells, marginal cells, intermediate cells, basal cells, inner pillar cells, outer pillar cells, Claudius cells, inner border cells, inner phalangeal cells, or cells of the stria vascularis.

In some embodiments, an animal cell is a specialized cell of the cochlea. In some embodiments, an animal cell is a hair cell. In some embodiments, an animal cell is a cochlear inner hair cell or a cochlear outer hair cell. In some embodiments, an animal cell is a cochlear inner hair cell. In some embodiments, an animal cell is a cochlear outer hair cell.

In some embodiments, an animal cell is in vitro. In some embodiments, an animal cell is of a cell type which is endogenously present in an animal, e.g., in a primate and/or human. In some embodiments, an animal cell is an autologous cell obtained from an animal and cultured ex vivo.

### Methods

Among other things, the present disclosure provides methods. In some embodiments, a method comprises introducing a composition as described herein into the inner ear (e.g., a cochlea) of a subject. For example, provided herein are methods that in some embodiments include administering to an inner ear (e.g., cochlea) of a subject (e.g., an animal, e.g., a mammal, e.g., a primate, e.g., a human) a therapeutically effective amount of any composition described herein. In some embodiments of any of these methods, the subject has been previously identified as having a defective inner ear cell target gene (e.g., a supporting and/or hearing cell target gene having a mutation that results in a decrease in the expression and/or activity of a supporting and/or hearing cell target protein encoded by the gene). Some embodiments of any of these methods further include, prior to the introducing or administering step, determining that the subject has a defective inner ear cell target gene. Some embodiments of any of these methods can further include detecting a mutation in an inner ear cell target gene in a subject. Some embodiments of any of the methods can further include identifying or diagnosing a subject as having nonsyndromic or syndromic sensorineural hearing loss.

In some embodiments, provided herein are methods of correcting an inner ear cell target gene defect (e.g., a defect in GJB2) in an inner ear of a subject, e.g., an animal, e.g., a mammal, e.g., a primate, e.g., a human. In some embodiments, methods include administering to the inner ear of a subject a therapeutically effective amount of any of the compositions described herein, where the administering repairs and or ameliorates the inner ear cell target gene defect in any cell subset of the inner ear of a subject. In some embodiments, the inner ear target cell may be a sensory cell, e.g., a hair cell, and/or a non-sensory cell, e.g., a supporting cell, and/or all or any subset of inner ear cells.

Also provided herein are methods of increasing the expression level of an inner ear cell target protein in any subset of inner ear cells of a subject (e.g., an animal, e.g., a mammal, e.g., a primate, e.g., a human) that include: administering to the inner ear of the subject a therapeutically effective amount of any of the compositions described herein, where the administering results in an increase in the expression level of the inner ear cell target protein (e.g., connexin 26 protein) in any cell subset of the inner ear of a subject. In some embodiments, the inner ear target cell may be a sensory cell, e.g., a hair cell, and/or a non-sensory cell, e.g., a supporting cell, and/or all or any subset of inner ear cells.

Also provided herein are methods of treating hearing loss, e.g., nonsyndromic sensorineural hearing loss or syndromic sensorineural hearing loss, in a subject (e.g., an animal, e.g., a mammal, e.g., a primate, e.g., a human) identified as having a defective inner ear cell target gene that include: administering to the inner ear of the subject a therapeutically effective amount of any of the compositions described herein.

Also provided herein are methods of restoring synapses and/or preserving spiral ganglion nerves in a subject identified or diagnosed as having an inner ear disorder that include: administering to the inner ear of the subject a therapeutically effective amount of any of the compositions described herein.

Also provided herein are methods of reducing the size of, and/or restoring the vestibular aqueduct to an appropriate size. Also provided herein are methods of restoring endolymphatic pH to an appropriate and/or acceptable level in a subject identified or diagnosed as having an inner ear disorder that include: administering to the inner ear of the subject a therapeutically effective amount of any of the compositions described herein.

Also provided herein are methods that include administering to an inner ear of a subject a therapeutically effective amount of any of the compositions described herein.

Also provided herein are surgical methods for treatment of hearing loss (e.g., nonsyndromic sensorineural hearing loss or syndromic sensorineural hearing loss). In some embodiments, the methods include the steps of: introducing into a cochlea of a subject a first incision at a first incision point; and administering intra-cochlearly a therapeutically effective amount of any of the compositions provided herein. In some embodiments, the composition is administered to the subject at the first incision point. In some embodiments, the composition is administered to the subject into or through the first incision.

In some embodiments of any of the methods described herein, any composition described herein is administered to the subject into or through the cochlea oval window membrane. In some embodiments of any of the methods described herein, any of the compositions described herein is administered to the subject into or through the cochlea round window membrane. In some embodiments of any of the methods described herein, the composition is administered using a medical device capable of creating a plurality of incisions in the round window membrane. In some embodiments, the medical device includes a plurality of micro-needles. In some embodiments, the medical device includes a plurality of micro-needles including a generally circular first aspect, where each micro-needle has a diameter of at least about 10 microns. In some embodiments, the medical device includes a base and/or a reservoir capable of holding the composition. In some embodiments, the medical device includes a plurality of hollow micro-needles individually including a lumen capable of transferring the composition. In some embodiments, the medical device includes a means for generating at least a partial vacuum.

In some embodiments, technologies of the present disclosure are used to treat subjects with or at risk of hearing loss. For example, in some embodiments, a subject has an autosomal recessive hearing loss attributed to at least one pathogenic variant of GJB2. It will be understood by those in the art that many different mutations in GJB2 can result in a pathogenic variant. In some such embodiments, a pathogenic variant causes or is at risk of causing hearing loss.

In some embodiments, a subject experiencing hearing loss will be evaluated to determine if and where one or more mutations may exist that may cause hearing loss. In some such embodiments, the status of GJB2 gene products or function (e.g., via protein or sequencing analyses) will be evaluated. In some embodiments of any of the methods described herein, the subject or animal is a mammal, in some embodiments the mammal is a domestic animal, a farm animal, a zoo animal, a non-human primate, or a human. In some embodiments of any of the methods described herein, the animal, subject, or mammal is an adult, a teenager, a juvenile, a child, a toddler, an infant, or a newborn. In some embodiments of any of the methods described herein, the animal, subject, or mammal is 1-5, 1-10, 1-20, 1-30, 1-40, 1-50, 1-60, 1-70, 1-80, 1-90, 1-100, 1-110, 2-5, 2-10, 10-20, 20-30, 30-40, 40-50, 50-60, 60-70, 70-80, 80-90, 90-100, 100-110, 10-30, 10-40, 10-50, 10-60, 10-70, 10-80, 10-90, 10-100, 10-110, 20-40, 20-50, 20-60, 20-70, 20-80, 20-90, 20-100, 20-110, 30-50, 30-60, 30-70, 30-80, 30-90, 30-100, 40-60, 40-70, 40-80, 40-90, 40-100, 50-70, 50-80, 50-90, 50-100, 60-80, 60-90, 60-100, 70-90, 70-100, 70-110, 80-100, 80-110, or 90-110 years of age. In some embodiments of any of the methods described herein, the subject or mammal is 1, 2, 3, 4, 5, 6, 7, 8, 9, 10, or 11 months of age.

In some embodiments of any of the methods described herein, the methods result in improvement in hearing (e.g., any of the metrics for determining improvement in hearing described herein) in a subject in need thereof for at least 10 days, at least 15 days, at least 20 days, at least 25 days, at least 30 days, at least 35 days, at least 40 days, at least 45 days, at least 50 days, at least 55 days, at least 60 days, at least 65 days, at least 70 days, at least 75 days, at least 80 days, at least 85 days, at least 100 days, at least 105 days, at least 110 days, at least 115 days, at least 120 days, at least 5 months, at least 6 months, at least 7 months, at least 8 months, at least 9 months, at least 10 months, at least 11 months, or at least 12 months.

In some embodiments a subject (e.g., an animal, e.g., a mammal, e.g., a human) has or is at risk of developing syndromic or nonsyndromic sensorineural hearing loss. In some embodiments a subject (e.g., an animal, e.g., a mammal, e.g., a human) has been previously identified as having a mutation in a GJB2 gene. In some embodiments a subject (e.g., an animal, e.g., a mammal, e.g., a human) has any of the mutations in a GJB2 gene that are described herein or are known in the art to be associated with syndromic or nonsyndromic sensorineural hearing loss.

In some embodiments, a subject (e.g., an animal, e.g., a mammal, e.g., a human) has been identified as being a carrier of a mutation in a GJB2 gene (e.g., via genetic testing). In some embodiments, a subject (e.g., an animal, e.g., a mammal, e.g., a human) has been identified as having a mutation in a GJB2 gene and has been diagnosed with syndromic or nonsyndromic sensorineural hearing loss. In some embodiments, a subject (e.g., an animal, e.g., a mammal, e.g., a human) has been identified as having syndromic or nonsyndromic sensorineural hearing loss.

In some embodiments, a subject (e.g., an animal, e.g., a mammal, e.g., a human) has been identified as being at risk of hearing loss (e.g., at risk of being a carrier of a gene mutation, e.g., a GJB2 mutation). In some such embodiments, a subject (e.g., an animal, e.g., a mammal, e.g., a human) may have certain risk factors of hearing loss or risk of hearing loss (e.g., known parental carrier, afflicted sibling, or symptoms of hearing loss). In some such embodiments, a subject (e.g., an animal, e.g., a mammal, e.g., a human) has been identified as being a carrier of a mutation in a GJB2 gene (e.g., via genetic testing) that has not previously been identified (i.e., is not a published or otherwise known variant of GJB2). In some such embodiments, identified mutations may be novel (i.e., not previously described in the literature), and methods of treatment for a subject suffering from or susceptible to hearing loss will be personalized to the mutation(s) of the particular patient.

In some embodiments, successful treatment of syndromic or nonsyndromic sensorineural hearing loss can be determined in a subject using any of the conventional functional hearing tests known in the art. Non-limiting examples of functional hearing tests are various types of audiometric assays (e.g., pure-tone testing, speech testing, test of the middle ear, auditory brainstem response, and otoacoustic emissions).

In some embodiments of any method provided herein, two or more doses of any composition described herein are introduced or administered into a cochlea of a subject. Some embodiments of any of these methods can include introducing or administering a first dose of a composition into a cochlea of a subject, assessing hearing function of the subject following introduction or administration of a first dose, and administering an additional dose of a composition into the cochlea of the subject found not to have a hearing function within a normal range (e.g., as determined using any test for hearing known in the art).

In some embodiments of any method provided herein, the composition can be formulated for intra-cochlear administration. In some embodiments of any of the methods described herein, the compositions described herein can be administered via intra-cochlear administration or local administration. In some embodiments of any of the methods described herein, the compositions are administered through the use of a medical device (e.g., any of the exemplary medical devices described herein).

In some embodiments, intra-cochlear administration can be performed using any of the methods described herein or known in the art. For example, in some embodiments, a composition can be administered or introduced into the cochlea using the following surgical technique: first using visualization with a 0 degree, 2.5-mm rigid endoscope, the external auditory canal is cleared and a round knife is used to sharply delineate an approximately 5-mm tympanomeatal flap. The tympanomeatal flap is then elevated and the middle ear is entered posteriorly. The chorda tympani nerve is identified and divided, and a curette is used to remove the scutal bone, exposing the round window membrane. To enhance apical distribution of the administered or introduced composition, a surgical laser may be used to make a small 2-mm fenestration in the oval window to allow for perilymph displacement during trans-round window membrane infusion of the composition. The microinfusion device is then primed and brought into the surgical field. The device is maneuvered to the round window, and the tip is seated within the bony round window overhang to allow for penetration of the membrane by the microneedle(s). The footpedal is engaged to allow for a measured, steady infusion of the composition. The device is then withdrawn and the round window and stapes foot plate are sealed with a gelfoam patch.

In some embodiments of any method provided herein, a subject has or is at risk of developing syndromic or nonsyndromic sensorineural hearing loss. In some embodiments of any method provided herein, a subject has been previously identified as having a mutation in an inner ear cell target gene, a gene which may be expressed in supporting cells and/or hair cells.

In some embodiments of any method provided herein, a subject has been identified as being a carrier of a mutation in an inner ear cell target gene (e.g., via genetic testing). In some embodiments of any method provided herein, a subject has been identified as having a mutation in an inner ear cell target gene and has been diagnosed with hearing loss (e.g., nonsyndromic sensorineural hearing loss or syndromic sensorineural hearing loss, e.g., DFNB1, DFNA3). Bart-Pumphrey syndrome, hystrix-like ichthyosis with deafness (HID), palmoplantar keratoderma with deafness, keratitis-ichthyosis-deafness (KID) syndrome, or Vohwinkel syndrome, respectively). In some embodiments of any of the methods described herein, the subject has been identified as having hearing loss (e.g., nonsyndromic sensorineural hearing loss or syndromic sensorineural hearing loss). In some embodiments, successful treatment of hearing loss (e.g., nonsyndromic sensorineural hearing loss or syndromic sensorineural hearing loss) can be determined in a subject using any of the conventional functional hearing tests known in the art. Non-limiting examples of functional hearing tests include various types of audiometric assays (e.g., pure-tone testing, speech testing, test of the middle ear, auditory brainstem response, and otoacoustic emissions).

In some embodiments, a subject cell is in vitro. In some embodiments, a subject cell is originally obtained from a subject and is cultured ex vivo. In some embodiments, a subject cell has previously been determined to have a defective inner ear cell target gene. In some embodiments, a subject cell has previously been determined to have a defective hair cell target gene. In some embodiments, a subject cell has previously been determined to have a defective supporting cell target gene.

In some embodiments of these methods, following treatment e.g., one or two or more administrations of compositions described herein, there is an increase in expression of an active inner ear cell target protein (e.g., connexin 26 protein). In some embodiments, an increase in expression of an active inner ear target protein as described herein (e.g., connexin 26 protein) is relative to a control level, e.g., as compared to the level of expression of an inner ear cell target protein prior to introduction of the compositions comprising any construct(s) as described herein.

Methods of detecting expression and/or activity of a target protein (e.g., connexin 26 protein) are known in the art. In some embodiments, a level of expression of an inner ear cell target protein can be detected directly (e.g., detecting inner ear cell target protein or target mRNA. Non-limiting examples of techniques that can be used to detect expression and/or activity of a target RNA or protein (e.g., a GJB2 gene product and/or connexin 26 protein or functional characteristic portion thereof) directly include: real-time PCR, Western blotting, immunoprecipitation, immunohistochemistry, mass spectrometry, or immunofluorescence. In some embodiments, expression of an inner ear cell target protein can be detected indirectly (e.g., through functional hearing tests).

Devices, Adninistration, and Surgical Methods

Provided herein are therapeutic delivery systems for treating hearing loss (e.g., nonsyndromic sensorineural hearing loss or syndromic sensorineural hearing loss). In one aspect, a therapeutic delivery system includes: i) a medical device capable of creating one or a plurality of incisions in a round window membrane of an inner ear of a subject in need thereof, and ii) an effective dose of a composition (e.g., any of the compositions described herein). In some embodiments, a medical device includes a plurality of micro-needles.

Also provided herein are surgical methods for treatment of hearing loss (e.g., nonsyndromic sensorineural hearing loss or syndromic sensorineural hearing loss). In some embodiments, a method the steps of: introducing into a cochlea of a subject a first incision at a first incision point; and administering intra-cochlearly a therapeutically effective amount of any of the compositions provided herein. In some embodiments, a composition is administered to a subject at the first incision point. In some embodiments, a composition is administered to a subject into or through the first incision.

In some embodiments of any method provided herein, any of the compositions described herein is administered to the subject into or through the cochlea oval window membrane. In some embodiments of any method provided herein, any of the compositions described herein is administered to the subject into or through the cochlea round window membrane. In some embodiments of any method provided herein, the composition is administered using a medical device capable of creating a plurality of incisions in the round window membrane. In some embodiments, a medical device includes a plurality of micro-needles. In some embodiments, a medical device includes a plurality of micro-needles including a generally circular first aspect, where each micro-needle has a diameter of at least about 10 microns. In some embodiments, a medical device includes a base and/or a reservoir capable of holding a composition. In some embodiments, a medical device includes a plurality of hollow micro-needles individually including a lumen capable of transferring a composition. In some embodiments, a medical device includes a means for generating at least a partial vacuum.

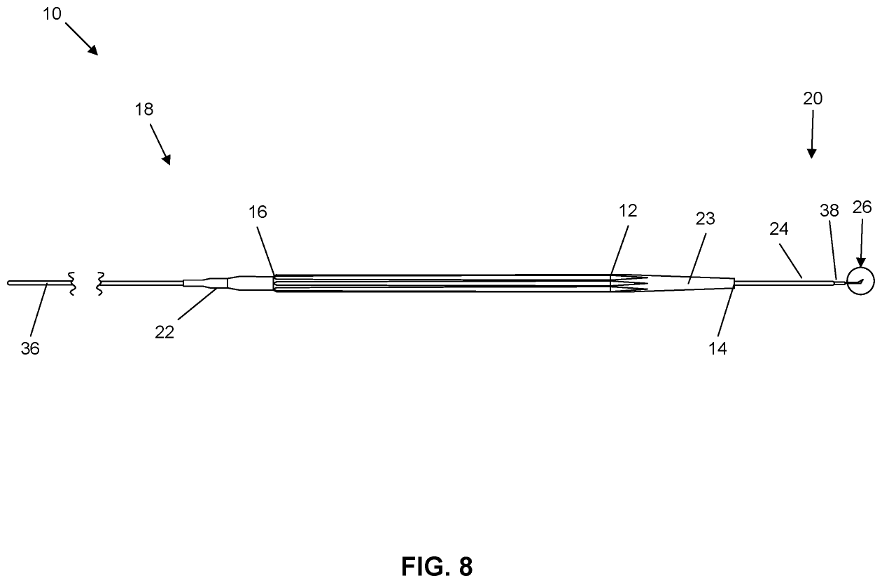

In some embodiments, the present disclosure describes a delivery approach that utilizes a minimally invasive, well-accepted surgical technique for accessing the middle ear and/or inner ear through the external auditory canal. The procedure includes opening one of the physical barriers between the middle and inner ear at the oval window, and subsequently using a device disclosed herein, e.g., as shown in FIGS. 8-11 (or microcatheter) to deliver a composition disclosed herein at a controlled flow rate and in a fixed volume, via the round window membrane.

In some embodiments, surgical procedures for mammals (e.g., rodents (e.g., mice, rats, hamsters, or rabbits), primates (e.g., NHP (e.g., macaque, chimpanzees, monkeys, or apes) or humans) may include venting to increase AAV vector transduction rates along the length of the cochlea. In some embodiments, absence of venting during surgery may result in lower AAV vector cochlear cell transduction rates when compared to AAV vector cochlear cell transduction rates following surgeries performed with venting. In some embodiments, venting facilitates transduction rates of about 75-100% of IHCs throughout the cochlea. In some embodiments, venting permits IHC transduction rates of about 50-70%, about 60-80%, about 70-90%, or about 80-100% at the base of the cochlea. In some embodiments, venting permits IHC transduction rates of about 50-70%, about 60-80%, about 70-90%, or about 80-100% at the apex of the cochlea.

A delivery device described herein may be placed in a sterile field of an operating room and the end of a tubing may be removed from the sterile field and connected to a syringe that has been loaded with a composition disclosed herein (e.g., one or more AAV vectors) and mounted in the pump. After appropriate priming of the system in order to remove any air, a needle may then be passed through the middle ear under visualization (surgical microscope, endoscope, and/or distal tip camera). A needle (or microneedle) may be used to puncture the RWM. The needle may be inserted until a stopper contacts the RWM. The device may then be held in that position while a composition disclosed herein is delivered at a controlled flow rate to the inner ear, for a selected duration of time. In some embodiments, the flow rate (or infusion rate) may include a rate of about 30 μL/min, or from about 25 μL/min to about 35 μL/min, or from about 20 μL/min to about 40 μL/min, or from about 20 μL/min to about 70 μL/min, or from about 20 μL/min to about 90 μL/min, or from about 20 μL/min to about 100 μL/min. In some embodiments, the flow rate is about 20 μL/min, about 30 μL/min, about 40 μL/min, about 50 μL/min, about 60 μL/min, about 70 μL/min, about 80 μL/min, about 90 μL/min or about 100 μL/min. In some embodiments, the selected duration of time (that is, the time during which a composition disclosed herein is flowing) may be about 3 minutes, or from about 2.5 minutes to about 3.5 minutes, or from about 2 minutes to about 4 minutes, or from about 1.5 minutes to about 4.5 minutes, or from about 1 minute to about 5 minutes. In some embodiments, the total volume of a composition disclosed herein that flows to the inner ear may be about 0.09 mL, or from about 0.08 mL to about 0.10 mL, or from about 0.07 mL to about 0.11 mL. In some embodiments, the total volume of a composition disclosed herein equates to from about 40% to about 50% of the volume of the inner ear.

Once the delivery has been completed, the device may be removed. In some embodiments, a device described herein, may be configured as a single-use disposable product. In other embodiments, a device described herein may be configured as a multi-use, sterilizable product, for example, with a replaceable and/or sterilizable needle sub-assembly. Single use devices may be appropriately discarded (for example, in a biohazard sharps container) after administration is complete.

In some embodiments, a composition disclosed herein comprises one or a plurality of rAAV constructs. In some embodiments, when more than one rAAV construct is included in the composition, the rAAV constructs are each different. In some embodiments, an rAAV construct comprises an anti-VEGF coding region, e.g., as described herein. In some embodiments, a composition comprises an rAAV particle comprising an AAV construct described herein. In some embodiments, the r AAV particle is encapsidated by an Anc80 capsid. In some embodiment, the Anc80 capsid comprises a polypeptide of SEQ ID NO: 44.

In some embodiments, a composition disclosed herein can be administered to a subject with a surgical procedure. In some embodiments, administration, e.g., via a surgical procedure, comprises injecting a composition disclosed herein via a delivery device as described herein into the inner ear. In some embodiments, a surgical procedure disclosed herein comprises performing a transcanal tympanotomy; performing a laser-assisted micro-stapedotomy; and injecting a composition disclosed herein via a delivery device as described herein into the inner ear.

In some embodiments, a surgical procedure comprises performing a transcanal tympanotomy; performing a laser-assisted micro-stapedotomy; injecting a composition disclosed herein via a delivery device as described herein into the inner ear; applying sealant around the round window and/or an oval window of the subject; and lowering a tympanomeatal flap of the subject to the anatomical position.

In some embodiments, a surgical procedure comprises performing a transcanal tympanotomy; preparing a round window of the subject; performing a laser-assisted micro-stapedotomy; preparing both a delivery device as described herein and a composition disclosed herein for delivery to the inner ear; injecting a composition disclosed herein via the delivery device into the inner ear; applying sealant around the round window and/or an oval window of the subject; and lowering a tympanomeatal flap of the subject to the anatomical position.

In some embodiments, performing a laser-assisted micro-stapedotomy includes using a KTP otologic laser and/or a CO2 otologic laser.

As another example, a composition disclosed herein is administered using a device and/or system specifically designed for intracochlear route of administration. In some embodiments, design elements of a device described herein may include: maintenance of sterility of injected fluid; minimization of air bubbles introduced to the inner ear; ability to precisely deliver small volumes at a controlled rate; delivery through the external auditory canal by the surgeon; minimization of damage to the round window membrane (RWM), or to inner ear, e.g., cochlear structures beyond the RWM; and/or minimization of injected fluid leaking back out through the RWM.

The devices, systems, and methods provided herein also describe the potential for delivering a composition safely and efficiently into the inner ear, in order to treat conditions and disorders that would benefit from delivery of a composition disclosed herein to the inner ear, including, but not limited to, hearing disorders, e.g., as described herein. As another example, by placing a vent in the stapes footplate and injecting through the RWM, a composition disclosed herein is dispersed throughout the cochlea with minimal dilution at the site of action. The development of the described devices allows the surgical administration procedure to be performed through the external auditory canal in humans. The described devices can be removed from the ear following infusion of an amount of fluid into the perilymph of the cochlea. In subjects, the device may be advanced through the external auditory canal, either under surgical microscopic control or along with an endoscope.

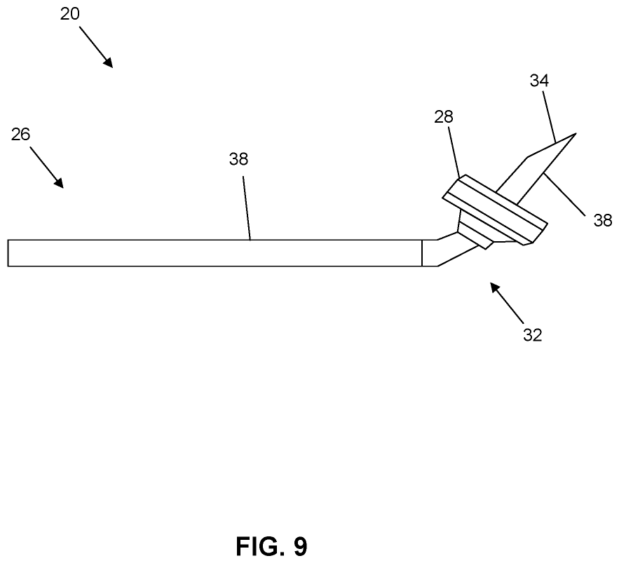

An exemplary device for use in any of the methods disclosed herein is described in FIGS. 8-11. FIG. 8 illustrates an exemplary device 10 for delivering fluid to an inner ear. Device 10 includes a knurled handle 12, and a distal handle adhesive 14 (for example, an epoxy such as Loctite 4014) that couples to a telescoping hypotube needle support 24. The knurled handle 12 (or handle portion) may include kurling features and/or grooves to enhance the grip. The knurled handle 12 (or handle portion) may be from about 5 mm to about 15 mm thick or from about 5 mm to about 12 mm thick, or from about 6 mm to about 10 mm thick, or from about 6 mm to about 9 mm thick, or from about 7 mm to about 8 mm thick. The knurled handle 12 (or handle portion) may be hollow such that fluid may pass through the device 10 during use. The device 10 may also include a proximal handle adhesive 16 at a proximal end 18 of the knurled handle 12, a needle sub-assembly 26 (shown in FIG. 9) with stopper 28 (shown in FIG. 34) at a distal end 20 of the device 10, and a strain relief feature 22. Strain relief feature 22 may be composed of a Santoprene material, a Pebax material, a polyurethane material, a silicone material, a nylon material, and/or a thermoplastic elastomer. The telescoping hypotube needle support 24 surrounds and supports a bent needle 38 (shown in FIG. 9) disposed therewithin.

Referring still to FIG. 8, the stopper 28 may be composed of a thermoplastic material or plastic polymer (such as a UV-cured polymer), as well as other suitable materials, and may be used to prevent the bent needle 38 from being inserted too far into the ear canal (for example, to prevent insertion of bent needle 38 into the lateral wall or other inner ear structure). Device 10 also may include a tapered portion 23 disposed between the knurled handle 12 and the distal handle adhesive 14 that is coupled to the telescoping hypotube needle support 24. The knurled handle 12 (or handle portion) may include the tapered portion 23 at the distal end of the handle portion 12. Device 10 may also include tubing 36 fluidly connected to the proximal end 16 the device 10 and acts as a fluid inlet line connecting the device to upstream components (for example, a pump, a syringe, and/or upstream components which, in some embodiments, may be coupled to a control system and/or power supply (not shown)). In some embodiments, the bent needle 38 (shown in FIG. 9) extends from the distal end 20, through the telescoping hypotube needle support 24, through the tapered portion 23, through the knurled handle 12, and through the strain relief feature 22 and fluidly connects directly to the tubing 36. In other embodiments, the bent needle 38 fluidly connects with the hollow interior of the knurled handle (for example, via the telescoping hypotube needle support 24) which in turn fluidly connects at a proximal end 16 with tubing 36. In embodiments where the bent needle 38 does not extend all the way through the interior of the device 10, the contact area (for example, between overlapping nested hypotubes 42), the tolerances, and/or sealants between interfacing components must be sufficient to prevent therapeutic fluid from leaking out of the device 10 (which operates at a relatively low pressure (for example, from about 1 Pascal to about 50 Pa, or from about 2 Pa to about 20 Pa, or from about 3 Pa to about 10 Pa)).

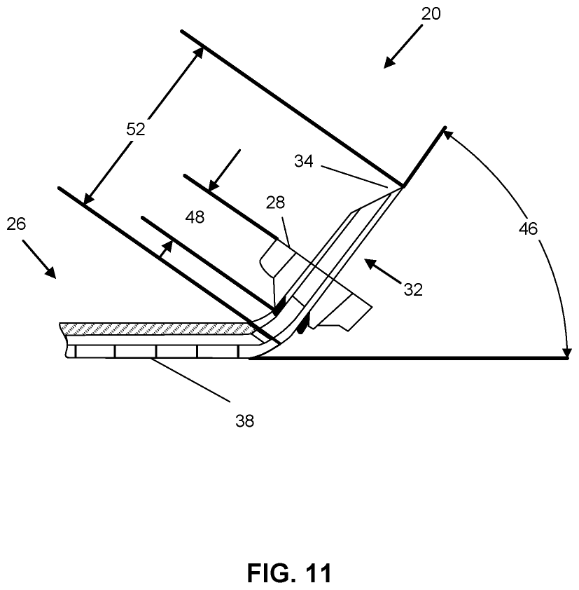

FIG. 9 illustrates a sideview of the bent needle sub-assembly 26, according to aspects of the present disclosed embodiments. Bent needle sub-assembly 26 includes a needle 38 that has a bent portion 32. Bent needle sub-assembly 26 may also include a stopper 28 coupled to the bent portion 32. The bent portion 32 includes an angled tip 34 at the distal end 20 of the device 10 for piercing a membrane of the ear (for example, the RWM). The needle 38, bent portion 32, and angled top 34 are hollow such that fluid may flow therethrough. The angle 46 (as shown in FIG. 11) of the bent portion 32 may vary. A stopper 28 geometry may be cylindrical, disk-shaped, annulus-shaped, dome-shaped, and/or other suitable shapes. Stopper 28 may be molded into place onto bent portion 32. For example, stopper 28 may be positioned concentrically around the bent portion 32 using adhesives or compression fitting. Examples of adhesives include an UV cure adhesive (such as Dymax 203A-CTH-F-T), elastomer adhesives, thermoset adhesives (such as epoxy or polyurethane), or emulsion adhesives (such as polyvinyl acetate). Stopper 28 fits concentrically around the bent portion 32 such that angled tip 34 is inserted into the ear at a desired insertion depth. The bent needle 38 may be formed from a straight needle using incremental forming, as well as other suitable techniques.

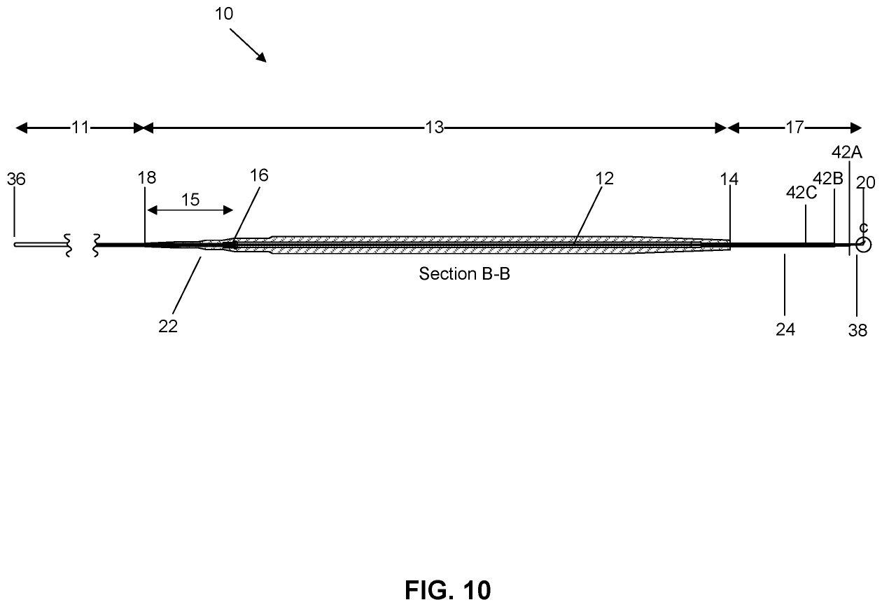

FIG. 10 illustrates a perspective view of exemplary device 10 for delivering fluid to an inner ear. Tubing 36 may be from about 1300 mm in length (dimension 11 in FIG. 10) to about 1600 mm, or from about 1400 mm to about 1500 mm, or from about 1430 mm to about 1450 mm. Strain release feature 22 may be from about 25 mm to about 30 mm in length (dimension 15 in FIG. 10), or from about 20 mm to about 35 mm in length. Handle 12 may be about 155.4 mm in length (dimension 13 in FIG. 10), or from about 150 mm to about 160 mm, or from about 140 mm to about 170 mm. The telescoping hypotube needle support 24 may have two or more nested hypotubes, for example three nested hypotubes 42A, 42B, and 42C, or four nested hypotubes 42A, 42B, 42C, and 42D. The total length of hypotubes 42A, 42B, 42C and tip assembly 26 (dimension 17 in FIG. 10) may be from about 25 mm to about 45 mm, or from about 30 mm to about 40 mm, or about 35 mm. In addition, telescoping hypotube needle support 24 may have a length of about 36 mm, or from about 25 mm to about 45 mm, or form about 30 mm to about 40 mm. The three nested hypotubes 42A, 42B, and 42C each may have a length of 3.5 mm, 8.0 mm, and 19.8 mm, respectively, plus or minus about 20%. The inner-most nested hypotube (or most narrow portion) of the telescoping hypotube needle support 24 may be concentrically disposed around needle 38.

FIG. 11 illustrates a perspective view of bent needle sub-assembly 26 coupled to the distal end 20 of device 10, according to aspects of the present disclosed embodiments. As shown in FIG. 11, bent needle sub-assembly 26 may include a needle 38 coupled to a bent portion 32. In other embodiments, the bent needle 38 may be a single needle (for example, a straight needle that is then bent such that it includes the desired angle 46). Needle 38 may be a 33-gauge needle, or may include a gauge from about 32 to about 34, or from about 31 to 35. At finer gauges, care must be taken to ensure tubing 36 is not kinked or damaged. Needle 38 may be attached to handle 12 for safe and accurate placement of needle 38 into the inner ear. As shown in FIG. 11, bent needle sub-assembly 26 may also include a stopper 28 disposed around bent portion 32. FIG. 11 also shows that bent portion 32 may include an angled tip 34 for piercing a membrane of the ear (for example, the RWM). Stopper 28 may have a height 48 of about 0.5 mm, or from about 0.4 mm to about 0.6 mm, or from about 0.3 mm to about 0.7 mm. Bent portion 32 may have a length 52 of about 1.45 mm, or from about 1.35 mm to about 1.55 mm, or from about 1.2 mm to about 1.7 mm. In other embodiments, the bent portion 32 may have a length greater than 2.0 mm such that the distance between the distal end of the stopper 28 and the distal end of the angled tip 34 is from about 0.5 mm to about 1.7 mm, or from about 0.6 mm to about 1.5 mm, or from about 0.7 mm to about 1.3 mm, or from about 0.8 mm to about 1.2 mm. FIG. 11 shows that stopper 28 may have a geometry that is cylindrical, disk-shaped, and/or dome-shaped. A person of ordinary skill will appreciate that other geometries could be used.

Evaluating Hearing Loss and Recovery

In some embodiments, hearing function is determined using auditory brainstem response measurements (ABR). In some embodiments, hearing is tested by measuring distortion product optoacoustic emissions (DPOAEs). In some such embodiments, measurements are taken from one or both ears of a subject. In some such embodiments, recordings are compared to prior recordings for the same subject and/or known thresholds on such response measurements used to define, e.g., hearing loss versus acceptable hearing ranges to be defined as normal hearing. In some embodiments, a subject has ABR and/or DPOAE measurements recorded prior to receiving any treatment. In some embodiments, a subject treated with one or more technologies described herein will have improvements on ABR and/or DPOAE measurements after treatment as compared to before treatment. In some embodiments, ABR and/or DPOAE measurements are taken after treatment is administered and at regular follow-up intervals post-treatment.

In some embodiments, hearing function is determined using speech pattern recognition or is determined by a speech therapist. In some embodiments, hearing function is determined by pure tone testing. In some embodiments, hearing function is determined by bone conduction testing. In some embodiments, hearing function is determined by acoustic reflex testing. In some embodiments hearing function is determined by tympanometry. In some embodiments, hearing function is determined by any combination of hearing analysis known in the art. In some such embodiments, measurements are taken holistically, and/or from one or both ears of a subject. In some such embodiments, recordings and/or professional analysis are compared to prior recordings and/or analysis for the same subject and/or known thresholds on such response measurements used to define, e.g., hearing loss versus acceptable hearing ranges to be defined as normal hearing. In some embodiments, a subject has speech pattern recognition, pure tone testing, bone conduction testing, acoustic reflex testing and/or tympanometry measurements and/or analysis conducted prior to receiving any treatment. In some embodiments a subject treated with one or more technologies described herein will have improvements on speech pattern recognition, pure tone testing, bone conduction testing, acoustic reflex testing and/or tympanometry measurements after treatment as compared to before treatment. In some embodiments, speech pattern recognition, pure tone testing, bone conduction testing, acoustic reflex testing and/or tympanometry measurements are taken after treatment is administered and at regular follow-up intervals post-treatment.

Methods of Characterizing

The term “mutation in a GJB2 gene” refers to a modification in a known consensus functional GJB2 gene that results in the production of a connexin 26 protein having one or more of: a deletion in one or more amino acids, one or more amino acid substitutions, and one or more amino acid insertions as compared to the consensus functional connexin 26 protein, and/or results in a decrease in the expressed level of the encoded connexin 26 protein in a mammalian cell as compared to the expressed level of the encoded connexin 26 protein in a mammalian cell not having a mutation. In some embodiments, a mutation can result in the production of a connexin 26 protein having a deletion in one or more amino acids (e.g., 2, 3, 4, 5, 6, 7, 8, 9, 10, 11, 12, 13, 14, 15 16, 17, 18, 19, 20, or more amino acids). In some embodiments, the mutation can result in a frameshift in the GJB2 gene. The term “frameshift” is known in the art to encompass any mutation in a coding sequence that results in a shift in the reading frame of the coding sequence. In some embodiments, a frameshift can result in a nonfunctional protein. In some embodiments, a point mutation can be a nonsense mutation (i.e., result in a premature stop codon in an exon of the gene). A nonsense mutation can result in the production of a truncated protein (as compared to a corresponding consensus functional protein) that may or may not be functional. In some embodiments, the mutation can result in the loss (or a decrease in the level) of expression of GJB2 mRNA or connexin 26 protein or both the mRNA and protein. In some embodiments, the mutation can result in the production of an altered connexin 26 protein having a loss or decrease in one or more biological activities (functions) as compared to a consensus functional connexin 26 protein.

In some embodiments, the mutation is an insertion of one or more nucleotides into a GJB2 gene. In some embodiments, the mutation is in a regulatory and/or control sequence of the connexin 26 gene, i.e., a portion of the gene that is not coding sequence. In some embodiments, a mutation in a regulatory and/or control sequence may be in a promoter or enhancer region and prevent or reduce the proper transcription of the GJB2 gene. In some embodiments, a mutation is in a known heterologous gene known to interact with a connexin 26 protein, or the GJB2 gene (e.g., GJB6, or other gap junction genes).

Methods of genotyping and/or detecting expression or activity of GJB2 mRNA and/or connexin 26 protein are known in the art (see e.g., Ito et al., World J Otorhinolaryngol. 2013 May 28; 3(2): 26-34, and Roesch et al., Int J Mol Sci. 2018 January; 19(1): 209, each of which is incorporated in its entirety herein by reference). In some embodiments, level of expression of GJB2 mRNA or connexin 26 protein may be detected directly (e.g., detecting connexin 26 protein, detecting GJB2 mRNA etc.). Non-limiting examples of techniques that can be used to detect expression and/or activity of GJB2 directly include, e.g., real-time PCR, quantitative real-time PCR, Western blotting, immunoprecipitation, immunohistochemistry, mass spectrometry, or immunofluorescence. In some embodiments, expression of GJB2 and/or connexin 26 protein can be detected indirectly (e.g., through functional hearing tests, ABRs, DPOAEs, etc.).

In some embodiments, tissue samples (e.g., comprising one or more inner ear cells, e.g., comprising one or more hair cells and/or one or more supporting cells) may be evaluated via morphological analysis to determine morphology of hair cells and/or support cells before and after administration of any agents (e.g., compositions, e.g., compositions comprising constructs, and/or particles, etc.) as described herein. In some such embodiments, standard immunohistochemical or histological analyses may be performed. In some embodiments, if cells are used in vitro or ex vivo, additional immunocytochemical or immunohistochemical analyses may be performed. In some embodiments, one or more assays of one or more proteins or transcripts (e.g., western blot, ELISA, polymerase chain reactions) may be performed on one or more samples from a subject or in vitro cell populations.

Production Methods

AAV systems are generally well known in the art (see, e.g., Kelleher and Vos, Biotechniques, 17(6):1110-17 (1994); Cotten et al., P.N.A.S. U.S.A., 89(13):6094-98 (1992); Curiel, Nat Immun, 13(2-3):141-64 (1994); Muzyczka, Curr Top Microbiol Immunol, 158:97-129 (1992); and Asokan A, et al., Mol. Ther., 20(4):699-708 (2012), each of which is incorporated in its entirety herein by reference). Methods for generating and using AAV constructs are described, for example, in U.S. Pat. Nos. 5,139,941, 4,797,368 and PCT filing application US2019/060328, each of which is incorporated in its entirety herein by reference.

Methods for obtaining viral constructs are known in the art. For example, to produce AAV constructs, the methods typically involve culturing a host cell which contains a nucleic acid sequence encoding an AAV capsid protein or fragment thereof, a functional rep gene; a recombinant AAV construct composed of AAV inverted terminal repeats (ITRs) and a coding sequence; and/or sufficient helper functions to permit packaging of the recombinant AAV construct into the AAV capsid proteins.

In some embodiments, components to be cultured in a host cell to package an AAV construct in an AAV capsid may be provided to the host cell in trans. Alternatively, any one or more components (e.g., recombinant AAV construct, rep sequences, cap sequences, and/or helper functions) may be provided by a stable host cell that has been engineered to contain one or more such components using methods known to those of skill in the art. In some embodiments, such a stable host cell contains such component(s) under the control of an inducible promoter. In some embodiments, such component(s) may be under the control of a constitutive promoter. In some embodiments, a selected stable host cell may contain selected component(s) under the control of a constitutive promoter and other selected component(s) under the control of one or more inducible promoters. For example, a stable host cell may be generated that is derived from HEK293 cells (which contain E1 helper functions under the control of a constitutive promoter), but that contain the rep and/or cap proteins under the control of inducible promoters. Other stable host cells may be generated by one of skill in the art using routine methods.

Recombinant AAV construct, rep sequences, cap sequences, and helper functions required for producing an AAV of the disclosure may be delivered to a packaging host cell using any appropriate genetic element (e.g., construct). A selected genetic element may be delivered by any suitable method known in the art, e.g., to those with skill in nucleic acid manipulation and include genetic engineering, recombinant engineering, and synthetic techniques (see, e.g., Sambrook et al., Molecular Cloning: A Laboratory Manual, Cold Spring Harbor Press, Cold Spring Harbor, N.Y., which is incorporated in its entirety herein by reference). Similarly, methods of generating AAV particles are well known and any suitable method can be used with the present disclosure (see, e.g., K. Fisher et al., J. Virol., 70:520-532 (1993) and U.S. Pat. No. 5,478,745, which are incorporated in their entirety herein by reference).

In some embodiments, recombinant AAVs may be produced using a triple transfection method (e.g., as described in U.S. Pat. No. 6,001,650, which is incorporated in its entirety herein by reference). In some embodiments, recombinant AAVs are produced by transfecting a host cell with a recombinant AAV construct (comprising a coding sequence) to be packaged into AAV particles, an AAV helper function construct, and an accessory function construct. An AAV helper function construct encodes “AAV helper function” sequences (i.e., rep and cap), which function in trans for productive AAV replication and encapsidation. In some embodiments, the AAV helper function construct supports efficient AAV construct production without generating any detectable wild-type AAV particles (i.e., AAV particles containing functional rep and cap genes). Non-limiting examples of constructs suitable for use with the present disclosure include pHLP19 (see, e.g., U.S. Pat. No. 6,001,650, which is incorporated in its entirety herein by reference) and pRep6cap6 construct (see, e.g., U.S. Pat. No. 6,156,303, which is incorporated in its entirety herein by reference). An accessory function construct encodes nucleotide sequences for non-AAV derived viral and/or cellular functions upon which AAV is dependent for replication (i.e., “accessory functions”). Accessory functions may include those functions required for AAV replication, including, without limitation, those moieties involved in activation of AAV gene transcription, stage specific AAV mRNA splicing, AAV DNA replication, synthesis of cap expression products, and AAV capsid assembly. Viral-based accessory functions can be derived from any known helper viruses such as adenovirus, herpesvirus (other than herpes simplex virus type-1), and vaccinia virus.

Additional methods for generating and isolating AAV viral constructs suitable for delivery to a subject are described in, e.g., U.S. Pat. Nos. 7,790,449; 7,282,199; WO 2003/042397; WO 2005/033321, WO 2006/110689; and U.S. Pat. No. 7,588,772, each of which is incorporated in its entirety herein by reference. In one system, a producer cell line is transiently transfected with a construct that encodes a coding sequence flanked by ITRs and a construct(s) that encodes rep and cap. In another system, a packaging cell line that stably supplies rep and cap is transiently transfected with a construct encoding a coding sequence flanked by ITRs. In each of these systems, AAV particles are produced in response to infection with helper adenovirus or herpesvirus, and AAVs are separated from contaminating virus. Other systems do not require infection with helper virus to recover the AAV—the helper functions (i.e., adenovirus E1, E2a, VA, and E4 or herpesvirus UL5, UL8, UL52, and UL29, and herpesvirus polymerase) are also supplied, in trans, by the system. In such systems, helper functions can be supplied by transient transfection of the cells with constructs that encode the helper functions, or the cells can be engineered to stably contain genes encoding the helper functions, the expression of which can be controlled at the transcriptional or posttranscriptional level.

In some embodiments, viral construct titers post-purification are determined. In some embodiments, titers are determined using quantitative PCR. In certain embodiments, a TaqMan probe specific to a construct is utilized to determine construct levels. In certain embodiments, the TaqMan probe is represented by SEQ ID NO: 58, while forward and reverse amplifying primers are exemplified by SEQ ID NO: 59 and 60 respectively.

### Exemplary Taqman Probe for Quantification of Constructs (SEQ ID NO: 58)

### /56-FAM/TCTGGCTCA/ZEN/CCGTCCTCTTCATTT/3IABkFQ/

Exemplary Forward qPCR Primer for Quantification of Constructs (SEQ ID NO: 59)

### CAAACACTCCACCAGCATTG

Exemplary Reverse qPCR Primer for Quantification of Constructs (SEQ ID NO: 60)

### CAGCCACAACGAGGATCATA

As described herein, in some embodiments, a viral construct of the present disclosure is an adeno-associated virus (AAV) construct. Several AAV serotypes have been characterized, including AAV1, AAV2, AAV3 (e.g., AAV3B), AAV4, AAV5, AAV6, AAV7, AAV8, AAV9, AAV10, AAV11, and AAV Anc80, as well as variants thereof. In some embodiments, an AAV particle is an AAV2/6, AAV2/8, AAV2/9, or AAV2/Anc80 particle (e.g., with AAV6, AAV8, AAV9, or Anc80 capsid and construct with AAV2 ITR). Other AAV particles and constructs are described in, e.g., Sharma et al., Brain Res Bull. 2010 Feb. 15; 81(2-3): 273, which is incorporated in its entirety herein by reference. Generally, any AAV serotype may be used to deliver a coding sequence described herein. However, the serotypes have different tropisms, e.g., they preferentially infect different tissues. In some embodiments, an AAV construct is a self-complementary AAV construct.

The present disclosure provides, among other things, methods of making AAV-based constructs. In some embodiments, such methods include use of host cells. In some embodiments, a host cell is a mammalian cell. A host cell may be used as a recipient of an AAV helper construct, an AAV minigene plasmid, an accessory function construct, and/or other transfer DNA associated with the production of recombinant AAVs. The term includes the progeny of an original cell that has been transfected. Thus, a “host cell” as used herein may refer to a cell that has been transfected with an exogenous DNA sequence. It is understood that the progeny of a single parental cell may not necessarily be completely identical in morphology or in genomic or total DNA complement as the original parent, due to natural, accidental, or deliberate mutation.

Additional methods for generating and isolating AAV particles suitable for delivery to a subject are described in, e.g., U.S. Pat. Nos. 7,790,449; 7,282,199; WO 2003/042397; WO 2005/033321, WO 2006/110689; and U.S. Pat. No. 7,588,772, each of which is incorporated in its entirety herein by reference. In one system, a producer cell line is transiently transfected with a construct that encodes a coding sequence flanked by ITRs and a construct(s) that encodes rep and cap. In another system, a packaging cell line that stably supplies rep and cap is transiently transfected with a construct encoding a coding sequence flanked by ITRs. In each of these systems, AAV particles are produced in response to infection with helper adenovirus or herpesvirus, and AAV particles are separated from contaminating virus. Other systems do not require infection with helper virus to recover the AAV particles—the helper functions (i.e., adenovirus E1, E2a, VA, and E4 or herpesvirus UL5, UL8, UL52, and UL29, and herpesvirus polymerase) are also supplied, in trans, by the system. In such systems, helper functions can be supplied by transient transfection of the cells with constructs that encode the helper functions, or the cells can be engineered to stably contain genes encoding the helper functions, the expression of which can be controlled at the transcriptional or posttranscriptional level.

In yet another system, a coding sequence flanked by ITRs and rep/cap genes are introduced into insect host cells by infection with baculovirus-based constructs. Such production systems are known in the art (see generally, e.g., Zhang et al., 2009, Human Gene Therapy 20:922-929, which is incorporated in its entirety herein by reference). Methods of making and using these and other AAV production systems are also described in U.S. Pat. Nos. 5,139,941; 5,741,683; 6,057,152; 6,204,059; 6,268,213; 6,491,907; 6,660,514; 6,951,753; 7,094,604; 7,172,893; 7,201,898; 7,229,823; and 7,439,065, each of which is incorporated in its entirety herein by reference.

### EXAMPLES

The disclosure is further described in detail by reference to the following experimental examples. These examples are provided for purposes of illustration only, and are not intended to be limiting unless otherwise specified. Thus, the disclosure should in no way be construed as being limited to the following examples, but rather should be construed to encompass any and all variations that become evident as a result of the teaching provided herein.

It is believed that one or ordinary skill in the art can, using the preceding description and following Examples, as well as what is known in the art, make and utilize technologies of the present disclosure.

### Example 1: Construction of Viral Constructs

This example provides a description of generating a viral construct as described herein. A recombinant AAV (rAAV) particle was generated by transfection with an adenovirus-free method as used by Xiao et al., J Virol. 73(5):3994-4003, 1999, which is incorporated in its entirety herein by reference. The cis plasmids with AAV ITRs, the trans plasmid with AAV Rep and Cap genes, and a helper plasmid with an essential region from an adenovirus genome were co-transfected in HEK293 cells. The rAAV construct expressed human connexin 26 under a single construct strategy using the constructs described. AAV Anc80 capsid was prepared to encapsulate a unique rAAV connexin 26 protein encoding construct.

Those of ordinary skill in the art will readily understand that similar constructs can be made in accordance with this example. For instance, rAAV constructs that express mammalian, primate, or human connexin 26 under single, dual, or multi construct strategies can be generated. AAV serotypes 1, 2, 3, 4, 5, 6, 7, 8, 9, rh8, rh10, rh39, rh43, and Anc80 can each be prepared to encapsulate four sets of connexin 26 constructs to test (i) a concatemerization-transplicing strategy, (ii) a hybrid intronic-homologous recombination-transplicing strategy, (iii) an exonic homologous recombination strategy, as summarized by Pryadkina et al., Meth. Clin. Devel. 2:15009, 2015, which is incorporated in its entirety herein by reference, and (iv) a single construct strategy. In some embodiments, a recombinant AAV (rAAV) particle is generated by transfection with an adenovirus-free method as used by Xiao et al., J Virol. 73(5):3994-4003, 1999, which is incorporated in its entirety herein by reference.

### Example 2: Generating and Purifying Viral Particles

This example provides a description of purification of a viral construct. A recombinant AAV (rAAV) is produced using a triple transfection protocol and purified. The fractions are analyzed by dot blot to determine those containing rAAV genomes. The viral genome number (vg) of each preparation is determined by a quantitative real-time PCR-based titration method using primers and probe corresponding to the ITR region of the AAV construct genome (Bartoli et al., Gene. Ther. 13:20-28, 2006, which is incorporated in its entirety herein by reference).

In some embodiments of this example, a recombinant AAV (rAAV) was produced using a standard triple transfection protocol and purified by two sequential cesium chloride (CsCl) density gradients, as described by Pryadkina et al., Mol. Ther. 2:15009, 2015, which is incorporated in its entirety herein by reference. At the end of second centrifugation, 11 fractions of 500 μl were recovered from the CsCl density gradient tube and purified through dialysis in 1×PBS. The fractions were analyzed by dot blot to determine those containing rAAV genomes. The viral genome number (vg) of each preparation was determined by a quantitative real-time PCR-based titration method using primers and probe corresponding to the ITR region of the AAV construct genome (Bartoli et al., Gene. Ther. 13:20-28, 2006, which is incorporated in its entirety herein by reference).

Those of ordinary skill in the art will readily understand that similar production and purifying processes can be conducted in accordance with this example. For instance, rAAV particles may be purified using various column chromatography methods known in the art, and/or viral genomes may be quantified using alternative primer sets.

### Example 3: Formulation of Viral Particles

This example relates to the preparation of compositions comprising rAAV particles, and a physiologically acceptable solution. An rAAV particle was produced and purified to a titer of 1.2×10¹³ vg/mL and was then prepared at dilutions of 6×10⁴, 1.3×10⁵, 1.8×10⁵, 4.5×10⁹, and 1.3×10¹⁰, vg/mL in a physiologically acceptable solution (e.g., commercially available 1×PBS with pluronic acid F68, prepared to a final concentration of: 8.10 mM Sodium Phosphate Dibasic, 1.5 mM Monopotassium Phosphate, 2.7 mM Potassium Chloride, 172 mM Sodium Chloride, and 0.001% Pluronic Acid F68).

In alternative embodiments, an rAAV is produced and purified to a known concentration (e.g., a titer of approximately 1×10³ vg/mL) and is then prepared at desired concentrations (e.g., dilutions of 6×10⁴, 1.3×10⁵, 1.8×10⁵, 4.5×10⁹, and 1.3×10¹⁰, vg/mL) in a physiologically acceptable buffer (e.g., commercially available 1×PBS with pluronic acid F68, prepared to a final concentration of: 8.10 mM Sodium Phosphate Dibasic, 1.5 mM Monopotassium Phosphate, 2.7 mM Potassium Chloride, 172 mM Sodium Chloride, and 0.001% Pluronic Acid F68; or e.g., artificial perilymph comprising NaCl, 120 mM; KCl, 3.5 mM; CaCl₂), 1.5 mM; glucose, 5.5 mM; HEPES, 20 mM. which is titrated with NaOH to adjust its pH to 7.5 (total Na⁺ concentration of 130 mM) as described in Chen et al., J Controlled Rel. 110:1-19, 2005, which is incorporated in its entirety herein by reference). Those of ordinary skill in the art will readily understand that alternative formulations can be prepared in accordance with this example. For instance, rAAV particles may be purified to an alternative titer, prepared at alternative dilutions, and suspended in alternative suitable solutions.

### Example 4: Device Description

This example relates to a device suitable for the delivery of rAAV particles to the inner ear. A composition comprising rAAV particles is delivered to the cochlea of a subject using a specialized microcatheter designed for consistent and safe penetration of the round window membrane (RWM). The microcatheter is shaped such that the surgeon performing the delivery procedure can enter the middle ear cavity via the external auditory canal and contact the end of the microcatheter with the RWM. The distal end of the microcatheter may include at least one microneedle with a diameter from about 10 microns to about 1,000 microns, which produces perforations in the RWM that are sufficient to allow a construct as described (e.g., an rAAV construct) to enter the cochlear perilymph of the scala tympani at a rate which does not damage the inner ear (e.g., a physiologically acceptable rate, e.g., a rate of approximately 30 μL/min to approximately 90 L/min), but small enough to heal without surgical repair. The remaining portion of the microcatheter, proximal to the microneedle(s), is loaded with the rAAV/artificial perilymph formulation at a defined titer (e.g., approximately 1×10¹² to 5×10³ vg/mL). The proximal end of the microcatheter is connected to a micromanipulator that allows for precise, low volume infusions of approximately 30 μL to approximately 100 μL.

### Example 5: In-Vitro Demonstration of GJB2 mRNA and Connexin 26 Protein Production (Anti-Connexin 26 Antibody)

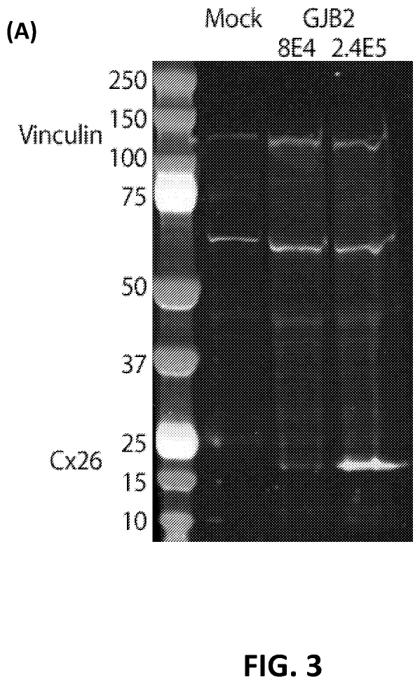

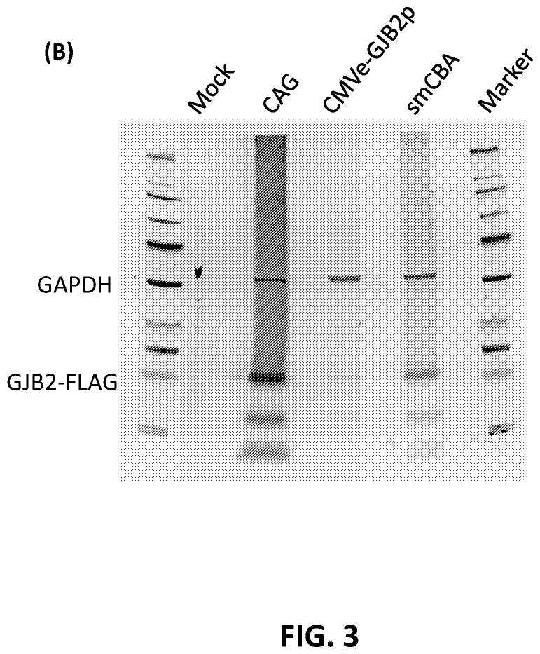

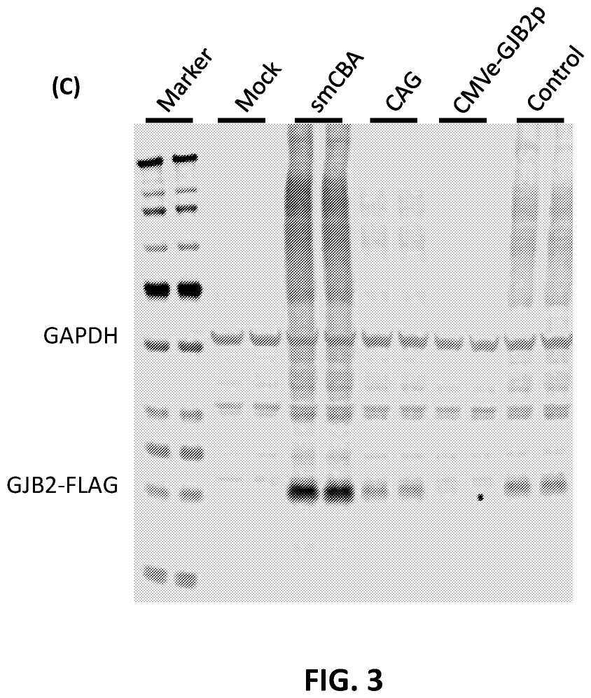

This example relates to the introduction, regulation, and expression analysis of rAAV constructs expressing a hGJB2 gene in mammalian cells grown in vitro or ex vivo. Mock rAAV particles, rAAV constructs, or rAAV particles comprising rAAV constructs (as represented by FIG. 2 panels (A)-(L)) encapsidated by Anc80 capsids were prepared and either transduced or transfected into cell culture using a known cellular concentration and either a known multiplicity of infection (MOI) (e.g., HEK293FT cells seeded at a density of 1.5×10⁵ cells per well at an MOI of 8.0×10⁴, 1.5×10⁵, 2.4×10⁵, or 3×10⁵ vg/cell per well in a 24 well format) or known DNA concentration (note, data shown for select constructs at select titers). Cells were harvested 48 hours post transfection or 72 hours post transduction using 100 μL RIPA buffer (Thermo Scientific) per well or 350 μL RLT Plus RNA lysis buffer (Qiagen). For protein expression analysis, thirty microliters of samples were loaded into individual wells in a 4-12% Bis-Tris protein gel and standard western blotting procedures as known in the art were conducted. Banding patterns were determined using a fluorescent reader, with test anti-connexin 26 (Thermo Scientific) and Vinculin or GAPDH as a control. The banding pattern of transgenic connexin 26 protein was determined (FIG. 3 panel (A), FIG. 3 panel (B), and FIG. 3 panel (C)). FIG. 3 panel (C) depicts banding patterns for protein isolated from HEK293FT cells that were transduced at an MOI of 3×10⁵ vg/cell per well with mock rAAV particles, AAVAnc80-CAG.5UTR.hGJB2.3F.3UTR (FIG. 2 panel (F), SEQ ID NO: 82), AAVAnc80-smCBA.5UTR.hGJB2.3F.3UTR (FIG. 2 panel (G), SEQ ID NO: 83), AAVAnc80-CMVeGJB2p.5UTR.hGJB2.3F.3UTR (FIG. 2 panel (H), SEQ ID NO: 84), or AAVAnc80-CAG.hGJB2.FLAG (FIG. 2 panel (A), SEQ ID NO: 45) as a positive control. As shown in FIG. 3 panel (C), a robust hGJB2 signal was detected using the CAG promoter, as well as the small-CBA promoter. A weaker but apparent band was also detected after expressing hGJB2 downstream of a custom CMV-enhancer/GJB2-promoter combination.

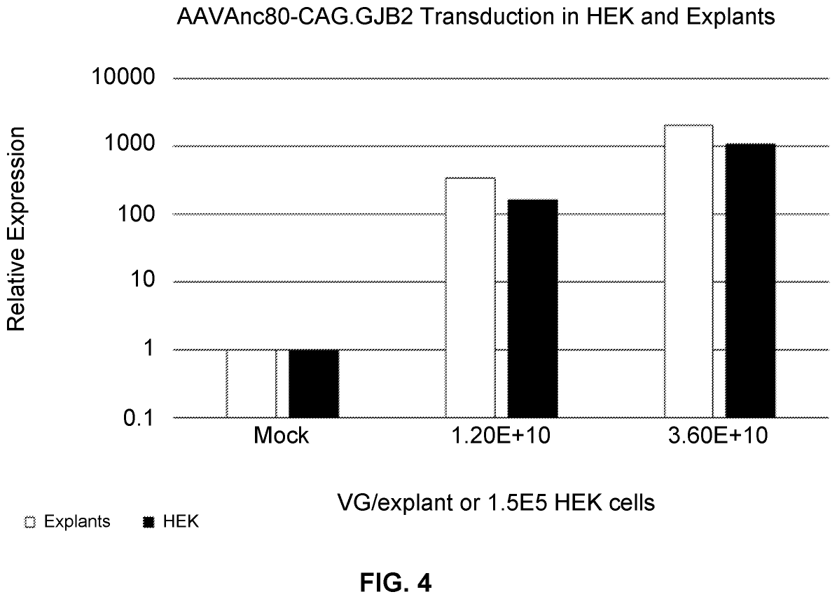

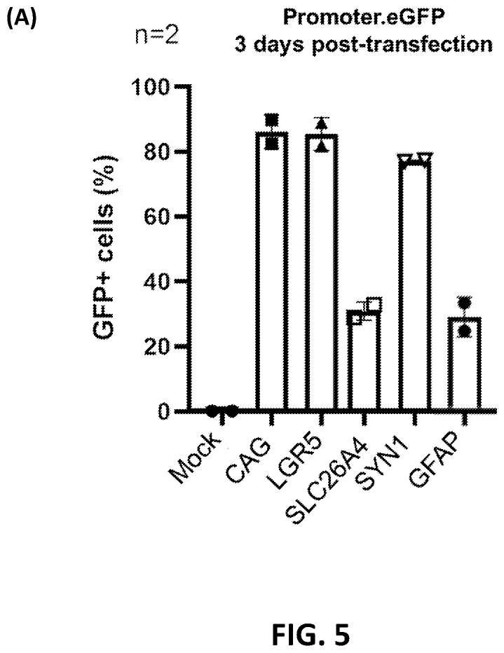

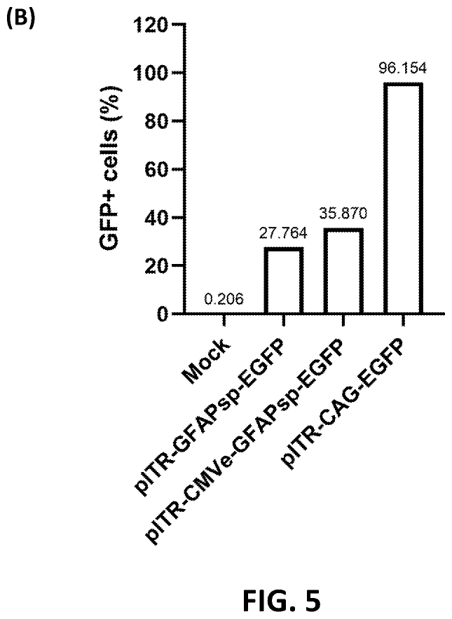

For RNA expression analysis. RNA was extracted using RNeasy Mini Kit (Qiagen). Relative mRNA expression levels were determined using quantitative real-time PCR with hGJB2 specific primers and TaqMan probe (SEQ ID NO: 58-60) and a human GAPDH TaqMan probe as control (Life Technologies). Robust and dose dependent GJB2 mRNA production was observed (FIG. 4).

Additionally, experiments were conducted to determine mRNA expression levels from rAAV constructs transduced into wild type explants (ex vivo). Mock rAAV particles or rAAV particles comprising rAAV constructs (as represented by FIG. 2 panels (A)-(E); data shown for select constructs at select titers) encapsidated by Anc80 capsids were prepared and transduced into explants at a MOI of 1.2×10¹⁰ or 3.6×10¹⁰ vg/cochlea. Cells were harvested 72 hours post transduction using 350 μL RLT Plus RNA lysis buffer (Qiagen), and RNA samples were prepared using the RNeasy Micro Kit (Qiagen). Relative mRNA expression levels were determined using quantitative real-time PCR with hGJB2 specific primers and TaqMan probe (SEQ ID NO: 58-60) and a human GAPDH TaqMan probe as control (Life Technologies). Robust and dose dependent GJB2 mRNA production was observed (FIG. 4).

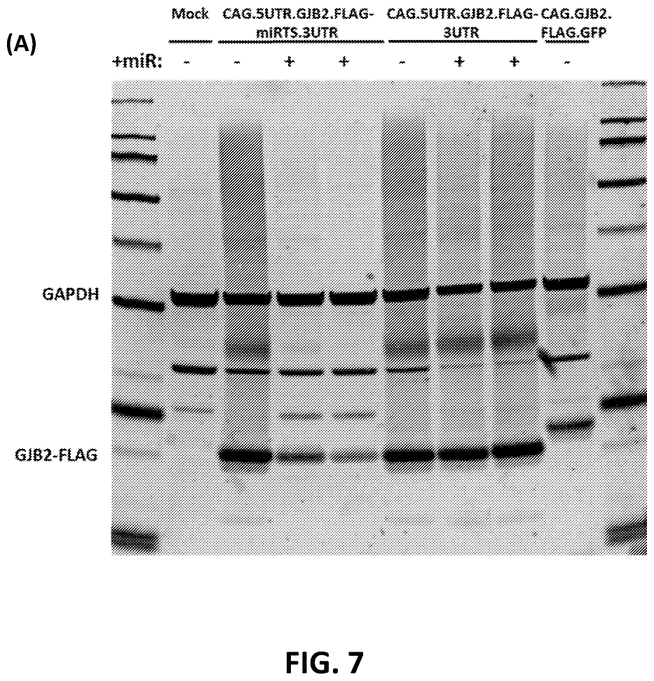

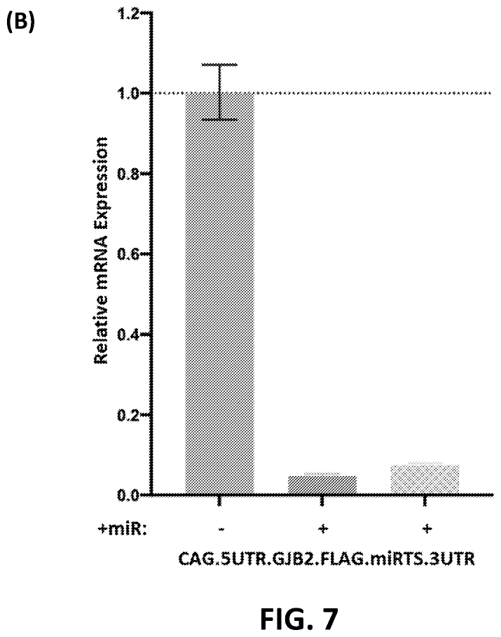

Further, experiments were conducted to demonstrate mRNA expression regulation from rAAV constructs transfected into HEK293FT cells. rAAV constructs comprising hGJB2.FLAG (CAG.5UTR.hGJB2.FLAG.3UTR; SEQ ID NO: 82) and optional miRNA regulatory target sites (miRTS) located in the 3′UTR (CAG.5UTR.hGJB2.FLAG.miRTS.3UTR; FIG. 2M; SEQ ID NO: 87) were transfected into HEK293FT cells at 300 ng with (+) or without (−) an additional plasmid comprising miRNA coding regions (e.g., miR-182, and miR-183) transfected at 400 ng. At 72 h post transfection the cells were harvested for GJB2 protein and RNA analysis using western blot analysis (see FIG. 7 panel (A)) and real-time qPCR (see FIG. 7 panel (B)). Reduction in GJB2 RNA and protein expression was detected in samples that were co-expressing the target plasmid and miR-182 and miR-183 compared to samples expressing the target plasmid alone. Similar hGJB2.FLAG comprising plasmids that did not include miR-182 and miR-183 target sites were used as control and presented similar hGJB2 protein levels with and without miR-182 and miR-183 co-expression (see FIG. 7 panel (A) and FIG. 7 panel (B)).

Those of ordinary skill in the art will readily understand that there are alternative methods of conducting the experiments associated with the current example, for instance, alternative viral titers, MOIs, cell concentrations, time to cellular harvest, reagents utilized for cellular harvesting or mRNA or protein analysis, AAV serotypes, and/or standard modifications to a construct comprising an SLC26A4 gene are practical and expected alterations of the current example.

### Example 6: Preliminary Hair Cell Tolerability Assessment of Transgenic GJB2 mRNA Expression and Connexin 26 Protein Production in Neonate Cochlear Explants

This example relates to the introduction, and expression analysis of rAAV constructs overexpressing a GJB2 gene in neonatal cochlear explants. Mock rAAV particles or rAAV particles comprising rAAV constructs (FIG. 2 panels (A)-(L)) encapsidated by Anc80 capsids are prepared and transduced into neonate cochlear explants at a known MOI (e.g., approximately 4.5×10⁹ or 1.3×10¹⁰ vg/per cochlea). Explants are grown to levels appropriate for harvest (e.g., for 72 hours post transduction), and are then prepared for immunofluorescence staining/imaging through fixation using 4% PFA or RNA extraction. RNA samples are prepared and GJB2 gene overexpression is confirmed using quantitative PCR with appropriate reagents in a manner described in a published method (e.g., appropriate according to the RNeasy Micro Kit and quantitative real-time PCR) using construct specific primers and relative to a control. Robust GJB2 mRNA production is observed in explants transduced with test rAAV when compared to mock transduction events. Tolerability and lack of hair cell toxicity is determined using immunofluorescence staining/imaging, antibodies targeting Myo7a (Proteus Biosciences) are utilized to depict inner ear hair cells, while DAPI staining is used to define nuclear positioning. No or low hair cell (Myo7) toxicity is observed after GJB2 overexpression.

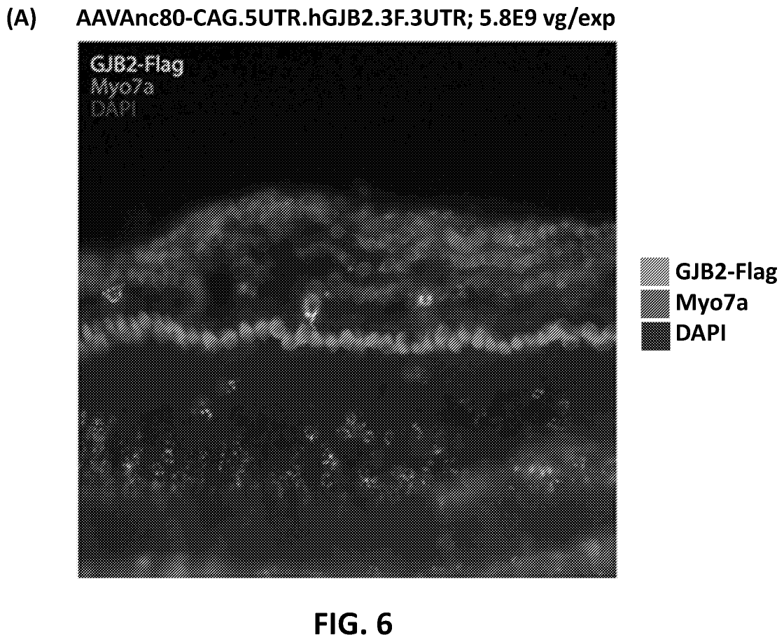

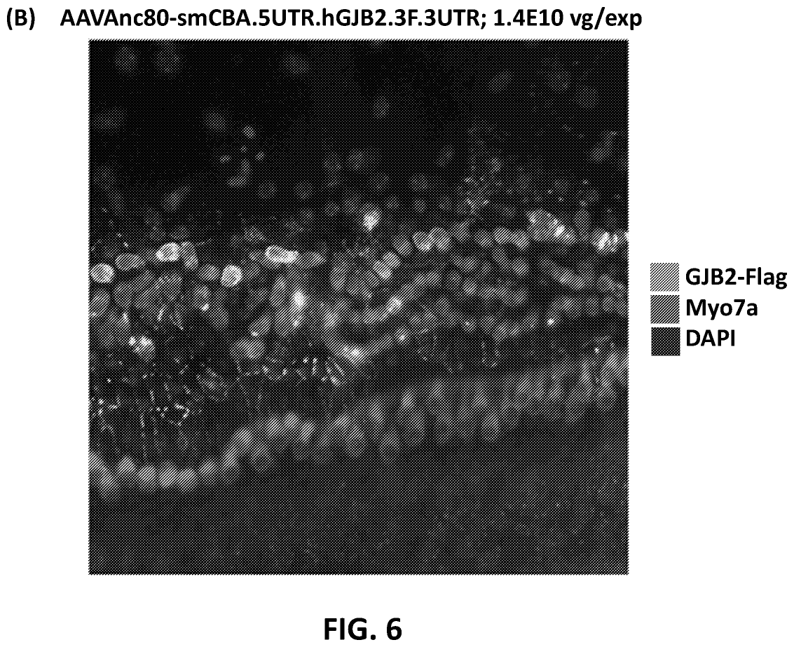

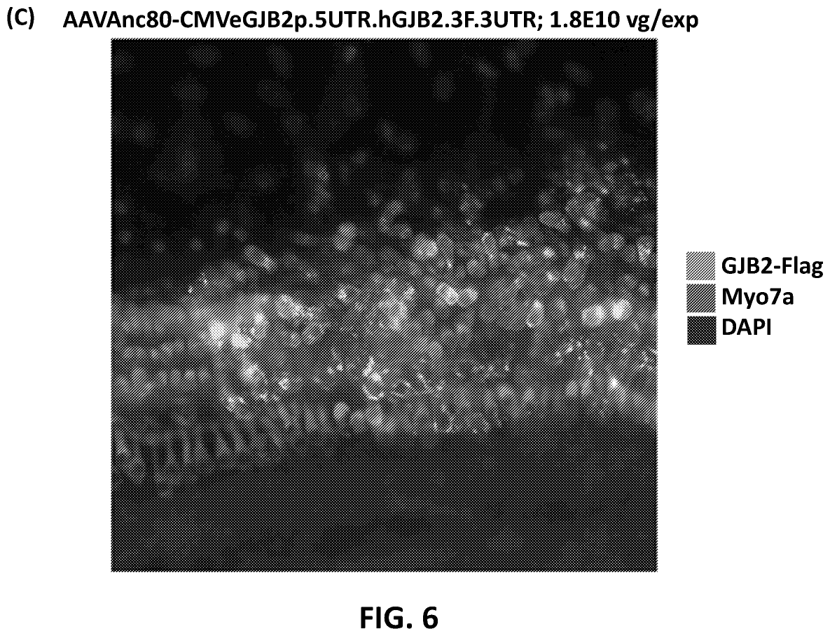

rAAV Anc80 particles comprising rAAV constructs driven by CAG, CMVe-GJB2p, or smCBA promoter/enhancer combinations were prepared and transduced into mouse neonate (P2) cochlear explants at a known MOI (approximately 5.8×10⁹, 1.4×10¹⁰, or 1.8×10¹⁰ vg/per cochlea respectively). Explants were grown to levels appropriate for harvest (e.g., for 72 hours post transduction), and were then prepared for immunofluorescence staining/imaging through fixation using 4% PFA. Explants were then DAPI stained (presented in blue) and immunostained using anti-FLAG antibodies (presented in green), and hair cell specific anti-Myo7a antibodies (presented in red), explants were subsequently imaged (exemplary data presented in FIG. 6). Robust supporting cell specific FLAG signal was observed in explants transduced with rAAV particles comprising AAVAnc80-CAG.5UTR.hGJB2.3F.3UTR (as depicted in FIG. 2 panel (F), SEQ ID NO: 82) at 5.8E9 vg/explant (see FIG. 6 panel (A)). Robust supporting cell specific FLAG signal was observed in explants transduced with rAAV particles comprising AAVAnc80-smCBA.5UTR.hGJB2.3F.3UTR (as depicted in FIG. 2 panel (G), SEQ ID NO: 83) at 1.4E10 vg/explant. Robust supporting cell specific FLAG signal was observed in explants transduced with rAAV particles comprising AAVAnc80-CMVeGJB2p.5UTR.hGJB2.3F.3UTR (as depicted in FIG. 2 panel (H), SEQ ID NO: 84) at 1.8E10 vg/explant. Variation in FLAG expression was detected across samples, likely the results of variability in vector titer.

### Example 7: Surgical Method in Aged Mice

The current example relates to the introduction of constructs described herein to the inner ear of aged mice. rAAV particles comprising an AAV capsid and a construct encoding a connexin 26 protein or characteristic functional portion thereof are prepared in formulation buffer (e.g., artificial perilymph or 1×PBS with pluronic acid F68) and then administered to the scala tympani in mice as described by Shu et al., Human Gene Therapy, 27(9):687-699, 2016, which is incorporated in its entirety herein by reference). Male and female mice older than P15 are anesthetized using an intraperitoneal injection of xylazine (e.g., approximately 5-10 mg/kg) and ketamine (e.g., approximately 90-120 mg/kg). Body temperature is maintained at 37° C. using an electric heating pad. An incision is made from the right post-auricular region and the tympanic bulla and posterior semicircular canal are exposed. The bulla is perforated with a surgical needle and the small hole is expanded to provide access to the cochlea. The bone of the cochlear lateral wall of the scala tympani is thinned with a dental drill so that the membranous lateral wall is left intact. A small hole is then drilled in the posterior semicircular canal (PSCC). Patency of the canalostomy is confirmed by visualization of a slow leak of perilymph. A Nanoliter Microinjection System in conjunction with glass micropipette is used to deliver a total of approximately 1 μL of construct containing buffer (e.g., rAAV constructs described herein at approximately 4.5×10⁹ to 5×10¹⁰ vg/per cochlea in artificial perilymph or 1×PBS with pluronic acid F68) to the scala tympani at a rate of approximately 2 nL/second. The glass micropipette is left in place for 5 minutes post-injection. Following cochleostomy and injection, the opening in the tympanic bulla and the PSCC are sealed with small pieces of fat, and the muscle and skin are sutured. The mice are allowed to awaken from anesthesia and their pain is controlled with 0.15 mg/kg buprenorphine hydrochloride for 3 days.

### Example 8: Transgenic Expression and Imaging of Connexin 26 Protein in Wild-Type Mice

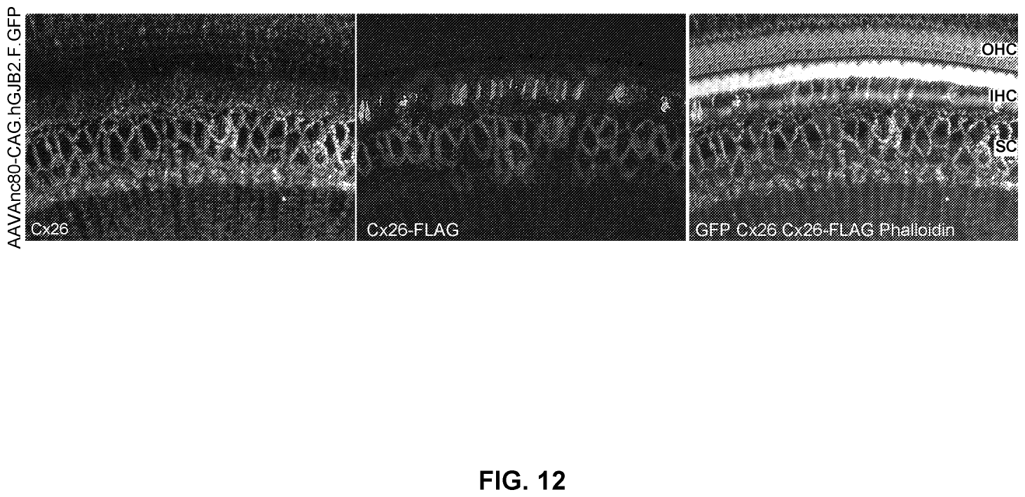

This example relates to the transgenic expression and analysis of transgenic connexin 26 protein in wild-type mice. Wild-type mice were administered AAVAnc80 particles (1.2×10¹⁰ vg/cochlea) comprising CAG.hGJB2.F.GFP (schematic provided in schematic provided in FIG. 2O) to the cochlea by the method described in Example 7. 10 days after administration clear and robust of exogenous Connexin 26 (FLAG; purple) was detected in the membrane of the supporting cells of the sensory epithelia (FIG. 12, middle and right panels). Expression of exogenous Connexin 26 was also detected in the inner hair cells. Endogenous Connexin 26 (red) was detected in all supporting cells (FIG. 12, left and right panels).

### Example 9: Transgenic Expression and Imaging of Connexin 26 Protein in Aged GJB2 Mutant Mice

This example relates to the transgenic expression and analysis of transgenic connexin 26 protein in adult mice. Suitable mutant GJB2 mice can be generated following temporally controlled tamoxifen induced knockout in Sox10-CreER×Cx26^{flox} lines, or CAG-CreER×Cx26^{flox} lines. Control and Mutant GJB2 mice aged are raised in accordance with animal welfare guidelines and approved by the Institutional Animal Care and Use Committee (IACUC), and surgical methods according to Example 7 are performed. Concurrent sham surgeries are performed as above with Anc80L65-GFP virus or vehicle as a negative control. At defined time points (e.g., 1 month, 2 month, 6 month, and 12 months post-surgery), mice are harvested for immunofluorescence staining/imaging. All harvested control and GJB2 mutant mice cochlear slices or whole-mount preparations are imaged using DAPI for nuclear expression, anti-Connexin 26 antibody, and anti-Myo7 or anti-phalloidin antibody.

### Example 10: Transgenic Expression and Imaging of Connexin 26 Protein in GJB2 Mutant Mice

This example relates to the transgenic expression and analysis of transgenic connexin 26 protein in neonatal mice. Suitable mutant GJB2 mice can be generated following temporally controlled tamoxifen induced knockout in Sox10-CreER×Cx26^{flox} lines, or CAG-CreER×Cx26^{flox} lines. Neonatal wild type or GJB2 mutant mice aged P0 to P4 are anesthetized (e.g., by hyperthermia on ice) to prepare for introduction of compositions described herein. Mock rAAV particles or rAAV constructs (as represented by FIG. 2 panels (A)-(L)) encapsidated by Anc80 capsids are prepared and introduced to the mouse inner ear through the round window membrane (RWM) or posterior semicircular canal (PSCC). Introduction of rAAV particles is performed through the following steps: A) preauricular incision to expose the cochlear bulla, B) glass micropipettes (cat #4878—WPI) pulled with a micropipette puller (cat #P87—Sutter instruments) to a final OD of about 10 μm are used to manually deliver (micropipettes held by a Nanoliter 2000 micromanipulator—WPI) compositions containing rAAV particles into the scala tympanic, which allows access to inner ear cells, C) approximately 1 μL of a composition described herein (e.g., rAAV constructs at approximately 4.5×10⁹ to 5×10¹⁰ vg/per cochlea) is injected into each tested cochlea at a release rate of approximately 0.3 μl/min (controlled by MICRO4 microinjection controller—WPI). Sham surgeries are performed as above with Anc80L65-GFP virus or vehicle as a negative control. Mice are allowed to recover from surgery without additional intervention. At day P21 mice physiology is evaluated. Subsequently or at a later time point after additional physiological assessments, the mice are harvested for immunofluorescence imaging. Control or GJB2 mutant mice cochlear sections or whole-mount preparations are imaged using DAPI for nuclear expression, anti-connexin 26 antibody, and anti-Myo7 or anti-phalloidin antibody.

### Example 11: Phenotypic Analysis of Transgenic Expression of GJB2 mRNA and Connexin 26 Protein in GJB2 Mutant Mice

The present example pertains to a phenotypic analysis of hearing in mice which are transgenically expressing GJB2 mRNA and connexin 26 protein in the inner ear. Suitable mutant GJB2 mice can be generated following temporally controlled tamoxifen induced knockout in Sox10-CreER×Cx26^{flox} lines, or CAG-CreER×Cx26^{flox} lines. Neonatal control and Mutant GJB2 mice aged P0 to P4 are anesthetized by hyperthermia on ice to prepare for introduction of compositions described herein. Vehicle controls, mock rAAV particles or rAAV constructs (as represented by FIG. 2 panels (A)-(L)) encapsidated by Anc80 capsids are prepared and introduced to the mouse inner ear through the round window membrane (RWM) or posterior semicircular canal (PSCC). Introduction of rAAV particles is performed through the following steps: A) preauricular incision to expose the cochlear bulla, B) glass micropipettes (cat #4878—WPI) pulled with a micropipette puller (cat #P87—Sutter instruments) to a final OD of about 10 μm were used to manually deliver (micropipettes held by a Nanoliter 2000 micromanipulator—WPI) compositions containing rAAV particles into the scala tympanic, which allows access to inner ear cells, C) 1 μL of a composition described herein (e.g., rAAV constructs at approximately 4.5×10⁹ to 5×10¹⁰ vg/per cochlea) is injected into each tested cochlea at a release rate of approximately 0.3 μl/min (controlled by MICRO4 microinjection controller—WPI). Sham surgeries are performed as above with Anc80L65-GFP virus or vehicle as a negative control. Mice are allowed recover from surgery without additional intervention

At defined test time points (e.g., 1 month, 2 month, 6 month, and 12 months post-surgery), control and mutant GJB2 mice which had undergone unilateral composition injection are anesthetized with sodium pentobarbital (e.g., approximately 35 mg/kg) delivered intraperitoneally. Mice are then placed and maintained in a head-holder within a grounded and acoustically and electrically insulated test room. An evoked potential detection system (e.g., Smart EP 3.90, Intelligent Hearing Systems, Miami, Fla., USA) is used to measure the thresholds of the auditory brainstem response (ABR) in mice. Click sounds as well as 8, 16, and 32 kHz tone bursts at varying intensity (from 10 to 130 dB SPL) are used to evoke ABRs in test mice. The response signals are recorded with a subcutaneous needle electrode inserted ventrolaterally into the ears of the mice. Sham injected mice act as a negative control while the mock-injected ear may act as an internal control for ABR tests, improvements in ABR performance is observed in test ears when compared to control ears and/or animals.

### Example 12: Phenotypic Analysis of Transgenic Expression of GJB2 mRNA and Connexin 26 Protein in Aged GJB2 Mutant Mice

This example relates to a phenotypic analysis of hearing in adult mice that are transgenically expressing connexin 26 protein. Suitable mutant GJB2 mice can be generated following temporally controlled tamoxifen induced knockout in Sox10-CreER×Cx26^{flox} lines, or CAG-CreER×Cx26^{flox} lines. Control and Mutant GJB2 mice are raised in accordance with animal welfare guidelines approved by the Institutional Animal Care and Use Committee (IACUC), and once of suitable age, surgical methods according to Example 7 are performed. Concurrent sham surgeries are performed as above with either vehicle formulation buffer or Anc80L65-GFP as a negative control. At defined time points (e.g., 1 month, 2 month, 6 month, and 12 months post-surgery), mice are anesthetized (e.g., with sodium pentobarbital at approximately 35 mg/kg or with xylazine at approximately 5-10 mg/kg and ketamine at approximately 90-120 mg/kg) delivered intraperitoneally. Mice are then placed and maintained in a head-holder within a grounded and acoustically and electrically insulated test room. An evoked potential detection system (Smart EP 3.90, Intelligent Hearing Systems, Miami, Fla., USA) is used to measure the thresholds of the auditory brainstem response (ABR) in mice. Click sounds as well as 8, 16, and 32 kHz tone bursts at varying intensity (from 10 to 130 dB SPL) are used to evoke ABRs in test mice. Response signals are recorded with a subcutaneous needle electrode inserted ventrolaterally into the ears of the mice. Improvements in hearing function are observed in exemplary results from aged GJB2 mutant mice which are unilaterally injected with compositions as described herein. Sham injected mice act as a negative control while the mock-injected ear may act as a control for ABR tests, improvements in ABR performance is observed in test ears when compared to control ears and/or animals.

### Example 13: Non-Invasive Prenatal Testing of Maternal Blood to Detect GJB2 Mutation

This example relates to the testing of maternal blood to determine an offspring's GJB2 genotype prior to birth to facilitate swift and efficacious therapeutic intervention. Maternal blood samples (20-40 mL) are collected into Cell-free DNA (cfDNA) tubes. At least 7 mL of plasma is isolated from each sample via a double centrifugation protocol of 2,000 g for 20 minutes, followed by 3,220 g for 30 minutes, with supernatant transfer following the first spin. cfDNA is isolated from 7-20 mL plasma using a QIAGEN QIAmp Circulating Nuclei Acid kit and eluted in 45 μL TE buffer. Pure maternal genomic DNA is isolated from the buffy coat obtained following the first centrifugation.

By combining thermodynamic modeling of the assays to select probes with minimized likelihood of probe-probe interaction with amplification approaches described previously (Stiller et al., 2009 Genome Res 19(10):1843-1848, which is incorporated in its entirety herein by reference), multiplexing of 11,000 assays can be achieved. Maternal cfDNA and maternal genomic DNA samples are pre-amplified for 15 cycles using 11,000 target-specific assays and an aliquot is transferred to a second PCR reaction of 15 cycles using nested primers. Samples are prepared for sequencing by adding barcoded tags in a third 12-cycle round of PCR. The targets include SNPs corresponding to the greater than 200 mutations in GJB2 known to lead DFNB1, DFNA3, Bart-Pumphrey syndrome, hystrix-like ichthyosis with deafness (HID), palmoplantar keratoderma with deafness, keratitis-ichthyosis-deafness (KID) syndrome, or Vohwinkel syndrome, and/or sequences that cover all exons of GJB2, in order to detect any presently unknown but potentially pathogenic variant. Optionally, sequences corresponding to other connexin genes which are amplified to identify possible heterologous digenic cases of DFNB1, DFNA3, Bart-Pumphrey syndrome, hystrix-like ichthyosis with deafness (HID), palmoplantar keratoderma with deafness, keratitis-ichthyosis-deafness (KID) syndrome, or Vohwinkel syndrome. The amplicons are then sequenced using an Illumina HiSeq sequencer. Genome sequence alignment is performed using commercially available software.

What is claimed is:

1. A construct comprising a coding sequence operably linked to a promoter, wherein the coding sequence encodes a connexin 26 protein.

2. The construct of claim 1, wherein the coding sequence is a GJB2 gene.

3. The construct of claim 2, wherein the GJB2 gene is a primate GJB2 gene.

4. The construct of claim 2 or 3, wherein the GJB2 gene is a human GJB2 gene.

5. The construct of claim 4, wherein the human GJB2 gene comprises a nucleic acid sequence according to SEQ ID NO: 1, SEQ ID NO: 2, SEQ ID NO: 3, or SEQ ID NO: 4.

6. The construct of claim 4 or 5, wherein the human GJB2 gene comprises a nucleic acid sequence according to SEQ ID NO: 1.

7. The construct of claim 1, wherein the connexin 26 protein is a primate connexin 26 protein.

8. The construct of claim 1 or 7, wherein the connexin 26 protein is a human connexin 26 protein.

9. The construct of claim 8, wherein the connexin 26 protein comprises an amino acid sequence according to SEQ ID NO: 7.

10. The construct of any one of claims 1-9, wherein the promoter is an inducible promoter, a constitutive promoter, a tissue-specific promoter, or a cell selective promoter for supporting.

11. The construct of any one of claims 1-10, wherein the promoter is an inner ear cell-specific promoter.

12. The construct of claim 11, wherein the promoter is an endogenous GJB2 gene promoter.

13. The construct of claim 12, wherein the promoter comprises a nucleic acid sequence according to SEQ ID NO: 17.

14. The construct of claim 11, wherein the inner ear cell-specific promoter is a GJB6 promoter, a SLC26A4 promoter, a TECTA promoter, a DFNA5 promoter, a COCH promoter, a NDP promoter, a SYN1 promoter, a GFAP promoter, a PLP promoter, a TAK1 promoter, a SOX21 promoter, a SOX2 promoter, a FGFR3 promoter, a PROX1 promoter, a GLAST1 promoter, a LGR5 promoter, a HES1 promoter, a HES5 promoter, a NOTCH1 promoter, a JAG1 promoter, a CDKN1A promoter, a CDKN1B promoter, a SOX10 promoter, a P75 promoter, a CD44 promoter, a HEY2 promoter, a LFNG promoter, a GDF6 promoter, a IGFBP2 promoter, a RBP7 promoter, a PARM1 promoter, a GJB2 minimal promoter, or a S100b promoter.

15. The construct of claim 11, wherein the promoter is capable of expressing the polynucleotide in an inner ear support cell selected from one or more of inner phalangeal cells/border cells (IPhC), inner pillar cells (IPC), outer pillar cells (OPC), Deiters' cells rows 1 and 2 (DC1/2), Deiters' cells row 3 (DC3), Hensen's cells (Hec), Claudius cells/outer sulcus cells (CC/OSC), interdental cells (Idc), inner sulcus cells (ISC), Kolliker's organ cells (KO), fibroblasts and other cells of the lateral wall, greater epithelial ridge cells (GER) (including lateral greater epithelial ridge cells (LGER)), and OC90+ cells (OC90).

16. The construct of claim 14, wherein the inner ear cell-specific promoter comprises a nucleic acid sequence with at least 85%, at least 90%, at least 95%, at least 96%, at least 97%, at least 98%, at least 99%, or 100% identity to any one of SEQ ID NOs: 16, 17, 61, 91, 54, 55, 56, 57, 62, 90, 95, 98, 101, and 104.

17. The construct of any preceding claim, wherein the constitutive promoter is a CAG promoter, a CBA promoter, a CMV promoter, or a CB7 promoter.

18. The construct of claim 17, wherein the promoter comprises a nucleic acid sequence with at least 85%, at least 90%, at least 95%, at least 96%, at least 97%, at least 98%, at least 99%, or 100% identity to SEQ ID NO: 10, 11, 12, 13, 14, and 15.

19. The construct of any of the preceding claims, further comprising a nucleic acid sequence comprising a microRNA regulatory target site (miRTS) for a microRNA expressed in an inner ear cell.

20. The construct of claim 19, wherein the microRNA is one or more of miR-194, miR-140, miR-18a, miR-99a, miR-30b, miR-15a, miR182, or miR-183.

21. The construct of claim 19, wherein the microRNA is expressed in one or more of inner ear hair cells, spiral ganglion cells, lateral supporting cells, basilar membrane cells, medial supporting cells, or spiral limbus cells.

22. The construct of claim 21, wherein the microRNA is expressed in an inner ear hair cell.

23. The construct of claim 22, wherein the microRNA is one or more of miR-194, miR-140, miR-18a, miR-99a, miR-30b, miR-15a, miR182, or miR-183.

24. The construct of claim 21, wherein the microRNA is expressed in spiral ganglion cells.

25. The construct of claim 24, wherein the microRNA is selected from one or more of miR-194, miR-18a, miR-99a, miR-30b, miR-15a, miR182, or miR-183.

26. The construct of claim 21, wherein the microRNA is expressed in lateral supporting cells.

27. The construct of claim 26, wherein the microRNA is selected from one or more of miR-99a, miR-30b, or miR-15a.

28. The construct of claim 21, wherein the microRNA is expressed in basilar membrane cells.

29. The construct of claim 28, the microRNA is selected from one or more of miR-99a, miR-30b, or miR-15a.

30. The construct of claim 21, wherein the microRNA is expressed in medial supporting cells.

31. The construct of claim 30, wherein the microRNA is selected from one or more of miR182 and miR-183.

32. The construct of claim 21, wherein the microRNA is expressed in spiral limbus cells.

33. The construct of claim 32, wherein the microRNA is selected from one or more of miR182 and miR-183.

34. The construct of any of claims 19-33, the microRNA regulatory target site comprises a nucleic acid sequence with least 85%, at least 90%, at least 95%, at least 96%, at least 97%, at least 98%, at least 99%, or 100% identity to any one of SEQ ID NOs: 78, 79, 107, 108, 109, 110, 111, or 112.

35. The construct of any of the preceding claims, wherein the construct further comprises a 5′ UTR.

36. The construct of claim 35, wherein the 5′ UTR comprises a nucleic acid sequence of any one of SEQ ID NOs: 20, 21, or 66.

37. The construct of any of the preceding claims, wherein the construct further comprises a 3′ UTR.

38. The construct of claim 37, wherein the 3′ UTR comprises a nucleic acid sequence of any one of SEQ ID NOs: 22, 67, 68, or 69.

39. The construct of any of claims 35-38, wherein the 3′ UTR and/or the 5′ UTR comprises the miRTS.

40. The construct of any of the preceding claims, further comprising a polyA tail.

41. The construct of claim 40, wherein the polyA tail is a bovine growth hormone, mouse-p-globin, mouse-α-globin, human collagen, polyoma virus, the Herpes simplex virus thymidine kinase gene (HSV TK), IgG heavy-chain gene, human growth hormone, or a SV40 late and early poly(A) site.

42. The construct of claim 41, wherein the polyA tail is a bovine growth hormone polyA.

43. The construct of any of the preceding claims, further comprising a 5′ and a 3′ inverted terminal repeat (ITR), wherein the 5′ ITR and the 3′ ITR flank the promoter and the polynucleotide.

44. The construct of claim 43, wherein the 5′ ITR and the 3′ ITR are AAV ITRs derived from a serotype selected from AAV1, AAV2, AAV3 (e.g., AAV3B), AAV4, AAV5, AAV6, AAV7, AAV8, AAV9, AAV10, AAV11, and AAV Anc80 ITRs.

45. The construct of claims 43-44, wherein the AAV ITRs are derived from serotype AAV2.

46. The construct of claims 43-45, wherein the 5′ AAV ITR comprises the nucleic acid sequence of SEQ ID NOs: 8 or 52.

47. The construct of claims 43-46, wherein the 3′ AAV ITR comprises the nucleic acid sequence of SEQ ID NOs: 9 or 53.

48. The construct of claims 46-47, wherein:
(i) a 5′ ITR comprising a nucleic acid sequence according to SEQ ID NO: 8 and a 3′ ITR comprising a nucleic acid sequence according to SEQ ID NO: 9; or
(ii) a 5′ ITR comprising a nucleic acid sequence according to SEQ ID NO: 52 and a 3′ ITR comprising a nucleic acid sequence according to SEQ ID NO: 52.

49. The construct of any one of claims 43-48, wherein (i) the 5′ ITR comprises the nucleic acid sequence of SEQ ID NOs: 8 or 52, (ii) the 5′ UTR comprises the nucleic acid of any one of SEQ ID NOs: 20, 21, or 66, (iii) the promoter comprises the nucleic acid sequence of any one of SEQ ID NOs: 10-17, 54, 55, 56, 57, 61, 62, 90, 91, 95, 98, 101, or 104, (iv) the 3′ UTR comprises the nucleic acid sequence of SEQ ID NOs: 22, 67, 68, or 69, and (v) the 3′ ITR comprises the nucleic acid sequence of SEQ ID NOs: 9 or 53.

50. The construct of claim 49, wherein the 3′ UTR and/or the 5′ UTR comprises the miRTS.

51. The construct of any of the preceding claims, wherein the construct comprises a nucleic acid sequence according to any one of SEQ ID NOs: 45-51, 50-51, 82-88, 94, 97, 100, 103, and 106.

52. The construct of any of the preceding claims, wherein the construct is an expression cassette.

53. A vector comprising the construct of any of the preceding claims.

54. The vector of claim 53, wherein the vector is a mammalian or viral vector.

55. The vector of claim 54, wherein the vector is a viral vector.

56. The vector of claim 55, wherein the viral vector is selected from the group consisting of an adeno-associated viral (AAV), adenovirus, or lentiviral vector.

57. The vector of claim 56, wherein the viral vector is an AAV vector.

58. An AAV particle comprising the construct of any of the preceding claims.

59. The AAV particle of claim 58, further comprising an AAV capsid, wherein the AAV capsid is or is derived from an AAV1, AAV2, AAV3 (e.g., AAV3B), AAV4, AAV5, AAV6, AAV7, AAV8, AAV9, AAV10, AAV11, and AAV Anc80 capsid.

60. An AAV particle of claim 59, wherein the AAV capsid is an AAV Anc80 capsid.

61. A composition comprising the construct of any one of claims 1-52, the vector of any one of claims 53-57, or the AAV particle of any of claims 58-60.

62. The composition of claim 61, wherein the composition is a pharmaceutical composition, optionally wherein the composition further comprises a pharmaceutically acceptable carrier.

63. The composition of claim 61 or 62, wherein the pharmaceutical composition is a synthetic perilymph solution.

64. An ex vivo cell comprising the construct of any one of claims 1-52, the vector of any one of claims 53-57, or the AAV particle of claims 58-60.

65. The ex vivo cell of claim 64, wherein the ex vivo cell is an inner ear cell.

66. The ex vivo cell of claim 65, wherein the ex vivo cell is an inner ear supporting cell.

67. The ex vivo cell of claim 66, wherein the inner ear supporting cells is selected from one or more of inner phalangeal cells/border cells (IPhC), inner pillar cells (IPC), outer pillar cells (OPC), Deiters' cells rows 1 and 2 (DC1/2), Deiters' cells row 3 (DC3), Hensen's cells (Hec), Claudius cells/outer sulcus cells (CC/OSC), interdental cells (Idc), inner sulcus cells (ISC), Kolliker's organ cells (KO), fibroblasts and other cells of the lateral wall, greater epithelial ridge cells (GER) (including lateral greater epithelial ridge cells (LGER)), and OC90+ cells (OC90).

68. A method comprising, transducing an ex vivo cell with:
(i) a construct of any one of claims 1-52 or the vector of any of claims X; and
(ii) one or more helper plasmids collectively comprising an AAV Rep gene, AAV Cap gene, AAV VA gene, AAV E2a gene, and AAV E4 gene.

69. The method of claim 68, wherein the ex vivo cell is an inner ear cell.

70. The method of claim 69, wherein the ex vivo cell is an inner ear supporting cell.

71. The method of claim 70, wherein the inner ear supporting cells is selected from one or more of inner phalangeal cells/border cells (IPhC), inner pillar cells (IPC), outer pillar cells (OPC), Deiters' cells rows 1 and 2 (DC1/2), Deiters' cells row 3 (DC3), Hensen's cells (Hec), Claudius cells/outer sulcus cells (CC/OSC), interdental cells (Idc), inner sulcus cells (ISC), Kolliker's organ cells (KO), fibroblasts and other cells of the lateral wall, greater epithelial ridge cells (GER) (including lateral greater epithelial ridge cells (LGER)), and OC90+ cells (OC90).

72. A method of expressing Connexin 26 in an inner ear supporting cell of a subject in need thereof, comprising administering the construct of any one of claims 1-52, the vector of any one of claims 53-57, the AAV particle of any one of claims 58-60, the composition of any one of claims 61-63, or the ex vivo cell of any one of claims 64-67 to the subject.

73. A method of increasing expression of Connexin 26 in an inner ear supporting cell of a subject in need thereof, comprising administering the construct of any one of claims 1-52, the vector of any one of claims 53-57, the AAV particle of any one of claims 58-60, the composition of any one of claims 61-63, or the ex vivo cell of any one of claims 64-67 to the subject.

74. The method of claims 72-73, wherein the expression of Connexin 26 is reduced, suppressed, or eliminated in non-inner ear supporting cells.

75. A method of decreasing expression of the Connexin 26 in non-inner ear supporting cells, comprising administering the construct of any one of claims 1-52, the vector of any one of claims 53-57, the AAV particle of any one of claims 58-60, the composition of any one of claims 61-63, or the ex vivo cell of any one of claims 64-67 to the subject.

76. The method of any one of claims 72-75, wherein the inner ear supporting cells is selected from one or more of inner phalangeal cells/border cells (IPhC), inner pillar cells (IPC), outer pillar cells (OPC), Deiters' cells rows 1 and 2 (DC1/2), Deiters' cells row 3 (DC3), Hensen's cells (Hec), Claudius cells/outer sulcus cells (CC/OSC), interdental cells (Idc), inner sulcus cells (ISC), Kolliker's organ cells (KO), fibroblasts and other cells of the lateral wall, greater epithelial ridge cells (GER) (including lateral greater epithelial ridge cells (LGER)), and OC90+ cells (OC90).

77. A method of reducing toxicity associated with expression of the Connexin 26 in an inner ear cell, comprising administering the construct of any one of claims 1-52, the vector of any one of claims 53-57, the AAV particle of any one of claims 58-60, the composition of any one of claims 61-63, or the ex vivo cell of any one of claims 64-67 to the subject.

78. The method of 77, wherein the inner ear cells are selected from inner ear hair cells, spiral ganglion cells, lateral supporting cells, basilar membrane cells, medial supporting cells, spiral limbus cells, inner sulcus cells, or any combination thereof.

79. A method of treating hearing loss in a subject suffering from or at risk of hearing loss, comprising administering the construct of any one of claims 1-52, the vector of any one of claims 53-57, the AAV particle of any one of claims 58-60, the composition of any one of claims 61-63, or the ex vivo cell of any one of claims 64-67 to the subject.

80. The method of claim 75-79, wherein expression of Connexin 26 is reduced, suppressed, or eliminated in inner ear hair cells, spiral ganglion cells, lateral supporting cells, basilar membrane cells, medial supporting cells, spiral limbus cells, inner sulcus cells, or any combination thereof.

81. The method of any one of claims 75-80, wherein toxicity due to expression of Connexin 26 is reduced in inner ear hair cells, spiral ganglion cells, lateral supporting cells, basilar membrane cells, medial supporting cells, spiral limbus cells, inner sulcus cells, or any combination thereof.

82. The method of claim 72-81, wherein Connexin 26 is predominately expressed in inner ear supporting cells.

83. The method of claim 82, wherein the inner ear supporting cells is selected from one or more of inner phalangeal cells/border cells (IPhC), inner pillar cells (IPC), outer pillar cells (OPC), Deiters' cells rows 1 and 2 (DC1/2), Deiters' cells row 3 (DC3), Hensen's cells (Hec), Claudius cells/outer sulcus cells (CC/OSC), interdental cells (Idc), inner sulcus cells (ISC), Kolliker's organ cells (KO), fibroblasts and other cells of the lateral wall, greater epithelial ridge cells (GER) (including lateral greater epithelial ridge cells (LGER)), and OC90+ cells (OC90).

84. The method of any one of claims 72-83, wherein administration is to the inner ear of the subject.

85. The method of claim 84, wherein the administration is to the cochlea of the subject.

86. The method of claim 85, wherein the administration is via a round window membrane injection.

87. The method of any one of claims 72-86 further comprising measuring a hearing level of the subject.

88. The method of claim 87, wherein a hearing level is measured by performing an auditory brainstem response (ABR) test.

89. The method of claim 87 or 88, further comprising comparing the hearing level of the subject to a reference hearing level.

90. The method of claim 89, wherein the reference hearing level is a published or historical reference hearing level.

91. The method of claim 89, wherein the hearing level of the subject is measured after the composition of claims 61-63 is introduced, and the reference hearing level is a hearing level of the subject that was measured before the composition of claims 61-63 was introduced.

92. The method of any one of claims 72-91, further comprising measuring a level of connexin 26 protein in the subject.

93. The method of claim 92, wherein the level of connexin 26 protein is measured in the inner ear of the subject.

94. The method of claim 92 or 93, wherein the level of connexin 26 protein is measured in the cochlea of the subject.

95. The method of any one of claims 92-94, further comprising comparing the level of connexin 26 protein in the subject to a reference connexin 26 protein level.

96. The method of claim 95, wherein the reference hearing level is a published or historical reference connexin 26 protein level.

97. The method of claim 95, wherein the level of connexin 26 protein in the subject is measured after the composition of claim 61-63 is introduced, and the reference connexin 26 protein level is a connexin 26 protein level of the subject that was measured before the composition of claim 61-63 was introduced.

98. Use of a construct of any one of claims 1-52, the vector of any of claims 53-57, an AAV particle of any one of claims 58-60, or a composition of any one of claims 61-63, or the ex vivo cell of any one of claims 64-67, for the treatment of hearing loss in a subject suffering from or at risk of hearing loss.

99. Use of a construct of any one of claims 1-52, the vector of any of claims 53-57, an AAV particle of any one of claims 58-60, or a composition of any one of claims 61-63, or the ex vivo cell of any one of claims 64-67n the manufacture of a medicament for the treatment of hearing loss.

100. A construct of any one of claims 1-52, the vector of any of claims 53-57, an AAV particle of any one of claims 58-60, or a composition of any one of claims 61-63, or the ex vivo cell of any one of claims 64-67, for use as a medicament.

101. A construct of any one of claims 1-52, the vector of any of claims 53-57, an AAV particle of any one of claims 58-60, or a composition of any one of claims 61-63, or the ex vivo cell of any one of claims 64-67, for use in the treatment of hearing loss.

102. A kit comprising 1-52, the vector of any of claims 53-57, an AAV particle of any one of claims 58-60, or a composition of any one of claims 61-63, or the ex vivo cell of any one of claims 64-67.

103. The kit of claim 102, wherein the wherein the construct, vector, AAV particle, composition or ex vivo cell is pre-loaded in a device.

104. The kit of claim 103, wherein the device is a microcatheter.

105. The kit of claim 104, wherein the microcatheter is shaped such that it can enter the middle ear cavity via the external auditory canal and contact the end of the microcatheter with the RWM.

106. The kit of claim 104 or 105, wherein a distal end of the microcatheter is comprised of at least one microneedle with diameter of between 10 and 1,000 microns.

107. The kit of claim 102, further comprising a device.

108. The kit of claim 107, wherein the device is a device described in any one of FIGS. 8-11.

109. The kit of claim 108, wherein the device comprises a needle comprising a bent portion and an angled tip.


SEQUENCE LISTING (112 sequences)

SEQ ID NO: 1 - DNA, 678 bp, Artificial Sequence
  Exemplary Human GJB2 cDNA coding Sequence
atggattggggcacgctgcagacgatcctggggggtgtgaacaaacactccaccagcattggaaagatctggctcaccgtcctcttcatttttcgcattatgatcctcgttgtggctgcaaaggaggtgtggggagatgagcaggccgactttgtctgcaacaccctgcagccaggctgcaagaacgtgtgctacgatcactacttccccatctcccacatccggctatgggccctgcagctgatcttcgtgtccacgccagcgctcctagtggccatgcacgtggcctaccggagacatgagaagaagaggaagttcatcaagggggagataaagagtgaatttaaggacatcgaggagatcaaaacccagaaggtccgcatcgaaggctccctgtggtggacctacacaagcagcatcttcttccgggtcatcttcgaagccgccttcatgtacgtcttctatgtcatgtacgacggcttctccatgcagcggctggtgaagtgcaacgcctggccttgtcccaacactgtggactgctttgtgtcccggcccacggagaagactgtcttcacagtgttcatgattgcagtgtctggaatttgcatcctgctgaatgtcactgaattgtgttatttgctaattagatattgttctgggaagtcaaaaaagccagtt

SEQ ID NO: 2 - DNA, 681 bp, Artificial Sequence
  Exemplary Human GJB2 cDNA coding Sequence
atggattggggcacgctgcagacgatcctggggggtgtgaacaaacactccaccagcattggaaagatctggctcaccgtcctcttcatttttcgcattatgatcctcgttgtggctgcaaaggaggtgtggggagatgagcaggccgactttgtctgcaacaccctgcagccaggctgcaagaacgtgtgctacgatcactacttccccatctcccacatccggctatgggccctgcagctgatcttcgtgtccacgccagcgctcctagtggccatgcacgtggcctaccggagacatgagaagaagaggaagttcatcaagggggagataaagagtgaatttaaggacatcgaggagatcaaaacccagaaggtccgcatcgaaggctccctgtggtggacctacacaagcagcatcttcttccgggtcatcttcgaagccgccttcatgtacgtcttctatgtcatgtacgacggcttctccatgcagcggctggtgaagtgcaacgcctggccttgtcccaacactgtggactgctttgtgtcccggcccacggagaagactgtcttcacagtgttcatgattgcagtgtctggaatttgcatcctgctgaatgtcactgaattgtgttatttgctaattagatattgttctgggaagtcaaaaaagccagtttaa

SEQ ID NO: 3 - DNA, 2290 bp, Artificial Sequence
  Exemplary spliced Human GJB2 isoform 1 cDNA including untranslated regions Sequence
gttgcggccccgcagcgcccgcgcgctcctctccccgactcggagcccctcggcggcgcccggcccaggacccgcctaggagcgcaggagccccagcgcagagaccccaacgccgagacccccgccccggccccgccgcgcttcctcccgacgcagagcaaaccgcccagagtagaagatggattggggcacgctgcagacgatcctggggggtgtgaacaaacactccaccagcattggaaagatctggctcaccgtcctcttcatttttcgcattatgatcctcgttgtggctgcaaaggaggtgtggggagatgagcaggccgactttgtctgcaacaccctgcagccaggctgcaagaacgtgtgctacgatcactacttccccatctcccacatccggctatgggccctgcagctgatcttcgtgtccacgccagcgctcctagtggccatgcacgtggcctaccggagacatgagaagaagaggaagttcatcaagggggagataaagagtgaatttaaggacatcgaggagatcaaaacccagaaggtccgcatcgaaggctccctgtggtggacctacacaagcagcatcttcttccgggtcatcttcgaagccgccttcatgtacgtcttctatgtcatgtacgacggcttctccatgcagcggctggtgaagtgcaacgcctggccttgtcccaacactgtggactgctttgtgtcccggcccacggagaagactgtcttcacagtgttcatgattgcagtgtctggaatttgcatcctgctgaatgtcactgaattgtgttatttgctaattagatattgttctgggaagtcaaaaaagccagtttaacgcattgcccagttgttagattaagaaatagacagcatgagagggatgaggcaacccgtgctcagctgtcaaggctcagtcgctagcatttcccaacacaaagattctgaccttaaatgcaaccatttgaaacccctgtaggcctcaggtgaaactccagatgccacaatggagctctgctcccctaaagcctcaaaacaaaggcctaattctatgcctgtcttaattttctttcacttaagttagttccactgagaccccaggctgttaggggttattggtgtaaggtactttcatattttaaacagaggatatcggcatttgtttctttctctgaggacaagagaaaaaagccaggttccacagaggacacagagaaggtttgggtgtcctcctggggttctttttgccaactttccccacgttaaaggtgaacattggttctttcatttgctttggaagttttaatctctaacagtggacaaagttaccagtgccttaaactctgttacactttttggaagtgaaaactttgtagtatgataggttattttgatgtaaagatgttctggataccattatatgttccccctgtttcagaggctcagattgtaatatgtaaatggtatgtcattcgctactatgatttaatttgaaatatggtcttttggttatgaatactttgcagcacagctgagaggctgtctgttgtattcattgtggtcatagcacctaacaacattgtagcctcaatcgagtgagacagactagaagttcctagtgatggcttatgatagcaaatggcctcatgtcaaatatttagatgtaattttgtgtaagaaatacagactggatgtaccaccaactactacctgtaatgacaggcctgtccaacacatctcccttttccatgactgtggtagccagcatcggaaagaacgctgatttaaagaggtcgcttgggaattttattgacacagtaccatttaatggggaggacaaaatggggcaggggagggagaagtttctgtcgttaaaaacagatttggaaagactggactctaaagtctgttgattaaagatgagctttgtctacttcaaaagtttgtttgcttaccccttcagcctccaattttttaagtgaaaatatagctaataacatgtgaaaagaatagaagctaaggtttagataaatattgagcagatctataggaagattgaacctgaatattgccattatgcttgacatggtttccaaaaaatggtactccacatatttcagtgagggtaagtattttcctgttgtcaagaatagcattgtaaaagcattttgtaataataaagaatagctttaatgatatgcttgtaactaaaataattttgtaatgtatcaaatacatttaaaacattaaaatataatctctataataa

SEQ ID NO: 4 - DNA, 2318 bp, Artificial Sequence
  Exemplary spliced Human GJB2 isoform X1 cDNA including untranslated regions Sequence
tttaggacccttgttcgcgaagaggtggtgtgcggctgagacccgcgtcctcaggacggttccatcagtgcctcgatcctgccccactggaggaggaaggcagcccgaacagcgctcacctaactaacagctgctgagagctgggttccgtggccatgcacctgggactgccttgagaagcgtgagcaaaccgcccagagtagaagatggattggggcacgctgcagacgatcctggggggtgtgaacaaacactccaccagcattggaaagatctggctcaccgtcctcttcatttttcgcattatgatcctcgttgtggctgcaaaggaggtgtggggagatgagcaggccgactttgtctgcaacaccctgcagccaggctgcaagaacgtgtgctacgatcactacttccccatctcccacatccggctatgggccctgcagctgatcttcgtgtccacgccagcgctcctagtggccatgcacgtggcctaccggagacatgagaagaagaggaagttcatcaagggggagataaagagtgaatttaaggacatcgaggagatcaaaacccagaaggtccgcatcgaaggctccctgtggtggacctacacaagcagcatcttcttccgggtcatcttcgaagccgccttcatgtacgtcttctatgtcatgtacgacggcttctccatgcagcggctggtgaagtgcaacgcctggccttgtcccaacactgtggactgctttgtgtcccggcccacggagaagactgtcttcacagtgttcatgattgcagtgtctggaatttgcatcctgctgaatgtcactgaattgtgttatttgctaattagatattgttctgggaagtcaaaaaagccagtttaacgcattgcccagttgttagattaagaaatagacagcatgagagggatgaggcaacccgtgctcagctgtcaaggctcagtcgctagcatttcccaacacaaagattctgaccttaaatgcaaccatttgaaacccctgtaggcctcaggtgaaactccagatgccacaatggagctctgctcccctaaagcctcaaaacaaaggcctaattctatgcctgtcttaattttctttcacttaagttagttccactgagaccccaggctgttaggggttattggtgtaaggtactttcatattttaaacagaggatatcggcatttgtttctttctctgaggacaagagaaaaaagccaggttccacagaggacacagagaaggtttgggtgtcctcctggggttctttttgccaactttccccacgttaaaggtgaacattggttctttcatttgctttggaagttttaatctctaacagtggacaaagttaccagtgccttaaactctgttacactttttggaagtgaaaactttgtagtatgataggttattttgatgtaaagatgttctggataccattatatgttccccctgtttcagaggctcagattgtaatatgtaaatggtatgtcattcgctactatgatttaatttgaaatatggtcttttggttatgaatactttgcagcacagctgagaggctgtctgttgtattcattgtggtcatagcacctaacaacattgtagcctcaatcgagtgagacagactagaagttcctagtgatggcttatgatagcaaatggcctcatgtcaaatatttagatgtaattttgtgtaagaaatacagactggatgtaccaccaactactacctgtaatgacaggcctgtccaacacatctcccttttccatgactgtggtagccagcatcggaaagaacgctgatttaaagaggtcgcttgggaattttattgacacagtaccatttaatggggaggacaaaatggggcaggggagggagaagtttctgtcgttaaaaacagatttggaaagactggactctaaagtctgttgattaaagatgagctttgtctacttcaaaagtttgtttgcttaccccttcagcctccaattttttaagtgaaaatatagctaataacatgtgaaaagaatagaagctaaggtttagataaatattgagcagatctataggaagattgaacctgaatattgccattatgcttgacatggtttccaaaaaatggtactccacatatttcagtgagggtaagtattttcctgttgtcaagaatagcattgtaaaagcattttgtaataataaagaatagctttaatgatatgcttgtaactaaaataattttgtaatgtatcaaatacatttaaaacattaaaatataatctctataataa

SEQ ID NO: 5 - DNA, 5469 bp, Artificial Sequence
  Exemplary Human GJB2 Genomic DNA Sequence
gttgcggccccgcagcgcccgcgcgctcctctccccgactcggagcccctcggcggcgcccggcccaggacccgcctaggagcgcaggagccccagcgcagagaccccaacgccgagacccccgccccggccccgccgcgcttcctcccgacgcaggtgagcccgccggccccggactgcccggccaggaacctggcgcggggagggaccgcgagacccagagcggttgcccggccgcgtgggtctcggggaaccggggggctggaccaacacacgtccttgggccggggggcgggggccgccttctggagcgggcgtttctgcggccgagctccggagctggaatggggcggccggggaagtggacgcgatggcaccgcccggggtgcgagtggggccgggcgcgcgcgggaggggaaaaaggcgcgggcgagccgccagcgcgaggtttgtggtgtcgccgatgtcccttcggggtactctagcgcagccgcctggctacttgacccactgccaccaaacgttttaaattcaccgaaagcttagcttcgaagcaaagctccgtttcgccggtgaagcaggaagccttcgctgcaggaactgacctttacctcttggagcggcttctgcagaaaaatccccgggcagagatttgggcggagtttgcctagaactaacgcggagccagccgatcccggcctaccccggggccaagatttcagtggcttccctttttcctaaacacttcacgagggtctgtttccgggctgtgctccccgcctagaaggaaaatttttaggacccttgttcgcgaagaggtggtgtgcggctgagacccgcgtcctcaggacggttccatcagtgcctcgatcctgccccactggaggaggaaggcagcccgaacagcgctcacctaactaacagctgctgagagctgggttccgtggccatgcacctgggactgccttgagaagcgtggtacggccgtgtccccatgtgaccttagagtccctttcgaaactgctgtgcacagtcggtcacaatttcagacactggtgagaagggtggaggaaccctctggggacagccaggcaaggtcgaccacccatcacctaagggtggagaaatttaaggggtgaagagtcccttttgccttttctggatcctggtgattcacctagtgtcttccctaaggaactgaaccaactcctccgctggcctctggcagccctccaggcggtgcaggatggcgtgggcccggtaggaagctgcatgtaaccgcccagggtcgggaggccaggagggcagctcctcctctgacttgaatattgaaaacaacttcgtcctgcttctgagcccctcttaacccatgaccccctagcccattggggagtaaatcttaatttactcctcttcctgaaaaaggatctttaaaacaggtagcttcaactcaagctttataaaataacaatatagggtttctcggaactgtatttttctcagctgatggtaactggacaggtctgtagaagggtgtatgacctgggtttggcaggtggaagagggcaaaggataaacccctcctcctgcagccccatattcttggccaggtgtattgttgtaaaccaggagagagtttacttcggggagtatcctgttttccactcagtgagggccaatgaagaatgtctaattccataagatgcttttgttaaaatcggaatgttgctgtcctcggtggttctgctgttgggacgggactggcctgagctgtgggtgctgtagcaggacaaccagctcacctaagggcctcccagtctggattatcaatgggtcagtgctgaacctgggctaaaatattgttttttccaatgatgttgtctttcccaagctcagtgaagctaaatgtttcacaggcctatgtcaatctgatgtaactttcgtggccacctctctcctgttagcctctgaccaaggtggcactggatggtttctgcctgaccttggtgccccgtggcagcgactgtgggtcatgaaagacattcactacgagcctgcttctggagtccatcagaaaacgggatgcaacttgcctaaaatgaggagaggaggatgcttttaagaaaaagaagaaggaggattcactaccagctctgaagggtggaaaagagatgattcatccggattgtggagagggtggaatcttgtttaggagagcgttggttgtggcaggcagggtgtaactatgaatcagtgaagacaattcacatcctgggatgaaaagaaggccatgggctcacaggagattatccactggcctctccacatccgcttgcagtaaggagtgtgggactctcccaagcttcagcgctgaactgcaatgcagtgacgtcgcttagctgggccagtaaccgagggagttgaattttctgtcattttaaaataatgtgtcttttaagaaacactttgaaattaaaaccacagcccacaattataatgcactgttgcagcacttatcaaaacagatatgctaactgagccatcagtgccagcctgacagtgaggccaccaagccatccacaaagcctacacgaaagtctgtgctcacagtggcttttctccatgaagagggcattcctaacctcttcctttcacgtaggaggaagcaaggtcctttgtaaaattttaactcggggtgcctcaaatgtaaacttaaccactggtaacaacagtttcactgctacatgccacgtctgtgaaaattcattcaagacattaaggaaagtggctcagcagagagactagacatcttatcctcacggttctcctgtacttggcctctcagcctttgagcaaggttggcccaagctagtatcggccccagtggtacagccaaaacttgagactgcaaatggatgcagctgttgaacgctgagtaacttctgcagagtcaggaagacccaaggaagctctgcagaggatgcaggggtacggtcagaacccctgagtgcctttcagctaacgaggactttatgacactccccagcacagcaaatttttatgatgtgtttaaagattgggtgaattactcaggtgaacaagctactttttatcagagaacacctaaaaacacgttcaagagggtttgggaactatacatttaatcctatgacaaactaagttggttctgtcttcacctgttttggtgaggttgtgtaagagttggtgtttgctcaggaagagatttaagcatgcttgcttacccagactcagagaagtctccctgttctgtcctagctagtgattcctgtgttgtgtgcattcgtcttttccagagcaaaccgcccagagtagaagatggattggggcacgctgcagacgatcctggggggtgtgaacaaacactccaccagcattggaaagatctggctcaccgtcctcttcatttttcgcattatgatcctcgttgtggctgcaaaggaggtgtggggagatgagcaggccgactttgtctgcaacaccctgcagccaggctgcaagaacgtgtgctacgatcactacttccccatctcccacatccggctatgggccctgcagctgatcttcgtgtccacgccagcgctcctagtggccatgcacgtggcctaccggagacatgagaagaagaggaagttcatcaagggggagataaagagtgaatttaaggacatcgaggagatcaaaacccagaaggtccgcatcgaaggctccctgtggtggacctacacaagcagcatcttcttccgggtcatcttcgaagccgccttcatgtacgtcttctatgtcatgtacgacggcttctccatgcagcggctggtgaagtgcaacgcctggccttgtcccaacactgtggactgctttgtgtcccggcccacggagaagactgtcttcacagtgttcatgattgcagtgtctggaatttgcatcctgctgaatgtcactgaattgtgttatttgctaattagatattgttctgggaagtcaaaaaagccagtttaacgcattgcccagttgttagattaagaaatagacagcatgagagggatgaggcaacccgtgctcagctgtcaaggctcagtcgctagcatttcccaacacaaagattctgaccttaaatgcaaccatttgaaacccctgtaggcctcaggtgaaactccagatgccacaatggagctctgctcccctaaagcctcaaaacaaaggcctaattctatgcctgtcttaattttctttcacttaagttagttccactgagaccccaggctgttaggggttattggtgtaaggtactttcatattttaaacagaggatatcggcatttgtttctttctctgaggacaagagaaaaaagccaggttccacagaggacacagagaaggtttgggtgtcctcctggggttctttttgccaactttccccacgttaaaggtgaacattggttctttcatttgctttggaagttttaatctctaacagtggacaaagttaccagtgccttaaactctgttacactttttggaagtgaaaactttgtagtatgataggttattttgatgtaaagatgttctggataccattatatgttccccctgtttcagaggctcagattgtaatatgtaaatggtatgtcattcgctactatgatttaatttgaaatatggtcttttggttatgaatactttgcagcacagctgagaggctgtctgttgtattcattgtggtcatagcacctaacaacattgtagcctcaatcgagtgagacagactagaagttcctagtgatggcttatgatagcaaatggcctcatgtcaaatatttagatgtaattttgtgtaagaaatacagactggatgtaccaccaactactacctgtaatgacaggcctgtccaacacatctcccttttccatgactgtggtagccagcatcggaaagaacgctgatttaaagaggtcgcttgggaattttattgacacagtaccatttaatggggaggacaaaatggggcaggggagggagaagtttctgtcgttaaaaacagatttggaaagactggactctaaagtctgttgattaaagatgagctttgtctacttcaaaagtttgtttgcttaccccttcagcctccaattttttaagtgaaaatatagctaataacatgtgaaaagaatagaagctaaggtttagataaatattgagcagatctataggaagattgaacctgaatattgccattatgcttgacatggtttccaaaaaatggtactccacatatttcagtgagggtaagtattttcctgttgtcaagaatagcattgtaaaagcattttgtaataataaagaatagctttaatgatatgcttgtaactaaaataattttgtaatgtatcaaatacatttaaaacattaaaatataatctctataataa

SEQ ID NO: 6 - DNA, 12513 bp, Artificial Sequence
  Exemplary expanded Human GJB2 Genomic DNA Sequence including certain regulatory regions
gactgtgaacttaaggcacagcagagctggggctgctcttaaggccctgctgtctctcctcttagtaacaacaccatttcacatgaagtgacagtggtatcttttgttgccctggaaatggaatacaacaatggctttccaacttttctgtggcagagacctacagacagaagtacattttacactggatccaggacacacatcagtctgaaaacacacacatgaaccaaacgtttcctaaagcattacttatccttgctaatagcaacacattctcatattcttttatacttcatttaatttcatataaaaaagaaaaggaaaggaaagaaatctatttctcagcccattaataaggtcaggagcagcaacaccagactagaagaaaagcttacctatagatttttctgccacctcttgagtgcgtccagctttccgacaagtctcagtgccatctactgtgcgctctgggtattgcaattgctttttttttttttttttttttttttttagaatgagactaagtcagagaacacaaagaacttctttccccacagtggagatggctctgaaagcgtttaaggaatagcttagatgagtggctaacacattctcccggttctgaattctaagaccacagactccatgtccagtccccaaagagaggctttgcaagctacagaatacccctctgactgggacctcaggagctaaactgaccacgtaattggttctagaaagtgaaacgttttaatttgaaacatccaaatgagcattttgtgaaaagctactgccgtccatcaaatacaacacagccagggagtcatcgctctattgcccttgtcaatcctacatctatagttttttttgctacagcagttcatgagtgttgactctattctaacttgttccagaagcccttcaagatgatagatagcacaatatttttgtagccagagctagaatgtagagctctttttggcttccttgtgaatgatccagaatttccatgttggcaagccaccataatttacagaatttactttttatattcaatagaagtaaaaaaaatttacctatttaaggagttatagctctggattcatttctgaccaaaatgtgctttttgacacaaatacaattggaaatgtctttgtaatttatccacagtctgcctagataatcataaaagaactgcatggatatatttgtgagtaagagcacgtgtccattcagcaaaaccaaggagatcaactaattctaccattgccttgaaacggagacacatctagcagtttgaatttcccccaaaagattgtatgtgtgaaaataagaatagaatgaggaaaatttaaaagcctatataataatttcagtcacaacttggcaattagaattttatgagatgtctttaatttggaagcaaagaacaattaaattattgaaggctggaatttttttttaactctttgaatggaacaacagattttccccaaaagatttgactttaacaattttcagaaaacataagtcagggtgtggttcaattacacagagagaaattgtagtgaaatagtgttccctgtaataattacccacaaaggagcacagtggagccactcctgcattaaaattacagtatcatatgtaagttattattaattaaccagagatgccaggagcttgtcagtttccaactgctattttgaggagagctaaagtttctctttttttgccagttattattattattaatatttcaacagcaaggcaagaaaagggaatgtggtccattaactaatggctcttgaaaagacactcaatgaatccaacttgccctaaaactgccaagtggtaggacagtctcttcgcgtcttgcatcattttctgccatcacctacgtgtgattcgtgagtcggaaattcaaccaagacatgtttaatgtatatttagagcattcttcccggcgggaattcacggtgccattccatcaggcagttggcaagcagtcacttgaaatattaagaaatatgatttgtgtcacactgatttattgcaaaacagcaacttctttctttttggttcatttataaaacaactgtcaaattaaaatgccaaatagctttaaacattagcattttcaccttataaccttacaagtgcatcactttaaacatctgagtaaaagttcagctcgatgacaatcacctgggatttacctgcatggtactaagcatatatgtaaaaatattactgatgggtatctctggcactctgaagtgacaaagtgtagccttcacagatctttgtcagttaatcatcaatagttacctgaaaagtgcccacttgccatcattcaagatcaaccaggcagacaccacagtgagttttccatcaaaaaaccttctctatctggtcagtctctgcacgtcaatgagacaaaggtgtatgctgcacgcagcagtactatcctaagctccctgtgtcctcaccatggggctgggtggctggggtggaggaacacaggattgggcttcagcttctctagggactggtacattaagagatgaagacataaaaggtgagaaaaacatggtttatttccaatgtttccatttctgttaaaagtaatgctttcaacagaaaaaaaatgcagcaatataagtgtgtaatttacaaaataatttcaggatttctttaatcattaatttgtggtgtcatctgttaactggatttacgtctaagctcatttgtaaataacttcaaatatccaagccttccctcacccttttcccacctcacctctcctccttctcctcccctacactggaggacactatgtacatgcatataatgtcctgccctagaggagtcctgagcctacttgggaagaaaacaccaactcacaggaaaacagcagaaatcacacaaaacagaataaaagcaagcgctgatctgtaagtgaagacttaagtgctataggacttccagctacaaatcctgaaaacacggagtggctgtgataatacgactagccaacatcacacagtaattttgcacataaggagaactaaatcaaagaaaacaaggaaaagaaagttgagcctataatcgtgatacaggcactaaaatctcaggtgacatttttcaatgggggaaagtcagtcaacttccgatctccaaaccatctttactagcgagcttcccacaatggttctagaaccttccttcattccaacccaaccaggattccaacagactcataaacaccacagcctttgagaaattaaagggagaacccaccaaccggcgccccactccccaccccaagtcacctctggctcaaccaagatgcgctcaggccaagaaagctgccccaccccacaggctttgcctgtcatttttaacaagccgactcagcacatctctcagatgggccatgcaaggcttttcgcagctcctggggctttgcctcttcatgagcagacactccctcttagactaagacctggagctggaaagtaggtggtaaccgcggtacaaaactcacgctcgtccctgcagaaactgcctaggtcggcccatggccacggggcgccaatttttcaaggaaaagtcaatgctaataatggtggcaatcacgggaaatccattctgaggccagatctgacttgtcaggattaatcatcatttccacttaacttcgaactgacctgggtaaaaacgtgagcgcgaggggaccaggctgcacctctgacctggctcccctctgcaaaaatcgcgaagtgggtgcccgaggtggggcgggggttgggggagacctccccgggagtccccacccagcctgctctgcacatcttagtccctcatccgcttgcgctgtgcaaatctgtcttctgtcatttgtatcgcaagacatcaaaatccccaaccaaatgcaaatactgagacctcataatctgagacaaagtttcacggtatccagaaagcccccagcaggtgtgcagtgcagagccagccccccagcggtcttccgcagaatcctatcagtttccccctttcgtgctgtgtgcatcgagcaggaaggggcttggcaggttttacctgccctctttcctttctgaaaagtctgggcctcctcaccccgaaaggagtcacctccttgcagttccccagttgcgaaaagaggaggaagttggctgggccgggggccgcggggggcaccctccgcagatggcgggacccccctgccggccatggcaaaaacgaggcttgtctctcccaccgcccccaaccttagtccttggcacattgttgaaagtaattgaataaaatcggaaattcgagaaggcgttcgttcggattggtgagattttgaggggagaaagaagcggggacttcgccggcaccagcggcgccccctcctcggccaccgttaacccccattccagagggcactgccccgccacccagcctaggtccccctgcgagagcctcgcgggcccgcgcagcctccgcgactcgaacagatcttcagtccttggaggaatgcctgtttctctaacaataaaaaattaaagaagcgctcataaatgccaagtcctctcgcactatgcggagtacagaggacaacgaccacagccatccctgaaccccgcccacggcacagcgccggagccggggtctggggcgccgcttcctggggggtcccgactctcagccgcccccgcttcacccgggccgccaaggggctgggggaggcggcgctcggggtaaccgggggagactcagggcgctgggggcacttggggaactcatgggggctcaaaggaactaggagatcgggacctcgaaggggacttggggggttcggggctttcgggggcggtcgggggttcgcggacccgggaagctctgaggacccagaggccgggcgcgctccgcccgcggcgccgccccctccgtaactttcccagtctccgagggaagaggcggggtgtggggtgcggttaaaaggcgccacggcgggagacaggtgttgcggccccgcagcgcccgcgcgctcctctccccgactcggagcccctcggcggcgcccggcccaggacccgcctaggagcgcaggagccccagcgcagagaccccaacgccgagacccccgccccggccccgccgcgcttcctcccgacgcaggtgagcccgccggccccggactgcccggccaggaacctggcgcggggagggaccgcgagacccagagcggttgcccggccgcgtgggtctcggggaaccggggggctggaccaacacacgtccttgggccggggggcgggggccgccttctggagcgggcgtttctgcggccgagctccggagctggaatggggcggccggggaagtggacgcgatggcaccgcccggggtgcgagtggggccgggcgcgcgcgggaggggaaaaaggcgcgggcgagccgccagcgcgaggtttgtggtgtcgccgatgtcccttcggggtactctagcgcagccgcctggctacttgacccactgccaccaaacgttttaaattcaccgaaagcttagcttcgaagcaaagctccgtttcgccggtgaagcaggaagccttcgctgcaggaactgacctttacctcttggagcggcttctgcagaaaaatccccgggcagagatttgggcggagtttgcctagaactaacgcggagccagccgatcccggcctaccccggggccaagatttcagtggcttccctttttcctaaacacttcacgagggtctgtttccgggctgtgctccccgcctagaaggaaaatttttaggacccttgttcgcgaagaggtggtgtgcggctgagacccgcgtcctcaggacggttccatcagtgcctcgatcctgccccactggaggaggaaggcagcccgaacagcgctcacctaactaacagctgctgagagctgggttccgtggccatgcacctgggactgccttgagaagcgtggtacggccgtgtccccatgtgaccttagagtccctttcgaaactgctgtgcacagtcggtcacaatttcagacactggtgagaagggtggaggaaccctctggggacagccaggcaaggtcgaccacccatcacctaagggtggagaaatttaaggggtgaagagtcccttttgccttttctggatcctggtgattcacctagtgtcttccctaaggaactgaaccaactcctccgctggcctctggcagccctccaggcggtgcaggatggcgtgggcccggtaggaagctgcatgtaaccgcccagggtcgggaggccaggagggcagctcctcctctgacttgaatattgaaaacaacttcgtcctgcttctgagcccctcttaacccatgaccccctagcccattggggagtaaatcttaatttactcctcttcctgaaaaaggatctttaaaacaggtagcttcaactcaagctttataaaataacaatatagggtttctcggaactgtatttttctcagctgatggtaactggacaggtctgtagaagggtgtatgacctgggtttggcaggtggaagagggcaaaggataaacccctcctcctgcagccccatattcttggccaggtgtattgttgtaaaccaggagagagtttacttcggggagtatcctgttttccactcagtgagggccaatgaagaatgtctaattccataagatgcttttgttaaaatcggaatgttgctgtcctcggtggttctgctgttgggacgggactggcctgagctgtgggtgctgtagcaggacaaccagctcacctaagggcctcccagtctggattatcaatgggtcagtgctgaacctgggctaaaatattgttttttccaatgatgttgtctttcccaagctcagtgaagctaaatgtttcacaggcctatgtcaatctgatgtaactttcgtggccacctctctcctgttagcctctgaccaaggtggcactggatggtttctgcctgaccttggtgccccgtggcagcgactgtgggtcatgaaagacattcactacgagcctgcttctggagtccatcagaaaacgggatgcaacttgcctaaaatgaggagaggaggatgcttttaagaaaaagaagaaggaggattcactaccagctctgaagggtggaaaagagatgattcatccggattgtggagagggtggaatcttgtttaggagagcgttggttgtggcaggcagggtgtaactatgaatcagtgaagacaattcacatcctgggatgaaaagaaggccatgggctcacaggagattatccactggcctctccacatccgcttgcagtaaggagtgtgggactctcccaagcttcagcgctgaactgcaatgcagtgacgtcgcttagctgggccagtaaccgagggagttgaattttctgtcattttaaaataatgtgtcttttaagaaacactttgaaattaaaaccacagcccacaattataatgcactgttgcagcacttatcaaaacagatatgctaactgagccatcagtgccagcctgacagtgaggccaccaagccatccacaaagcctacacgaaagtctgtgctcacagtggcttttctccatgaagagggcattcctaacctcttcctttcacgtaggaggaagcaaggtcctttgtaaaattttaactcggggtgcctcaaatgtaaacttaaccactggtaacaacagtttcactgctacatgccacgtctgtgaaaattcattcaagacattaaggaaagtggctcagcagagagactagacatcttatcctcacggttctcctgtacttggcctctcagcctttgagcaaggttggcccaagctagtatcggccccagtggtacagccaaaacttgagactgcaaatggatgcagctgttgaacgctgagtaacttctgcagagtcaggaagacccaaggaagctctgcagaggatgcaggggtacggtcagaacccctgagtgcctttcagctaacgaggactttatgacactccccagcacagcaaatttttatgatgtgtttaaagattgggtgaattactcaggtgaacaagctactttttatcagagaacacctaaaaacacgttcaagagggtttgggaactatacatttaatcctatgacaaactaagttggttctgtcttcacctgttttggtgaggttgtgtaagagttggtgtttgctcaggaagagatttaagcatgcttgcttacccagactcagagaagtctccctgttctgtcctagctagtgattcctgtgttgtgtgcattcgtcttttccagagcaaaccgcccagagtagaagatggattggggcacgctgcagacgatcctggggggtgtgaacaaacactccaccagcattggaaagatctggctcaccgtcctcttcatttttcgcattatgatcctcgttgtggctgcaaaggaggtgtggggagatgagcaggccgactttgtctgcaacaccctgcagccaggctgcaagaacgtgtgctacgatcactacttccccatctcccacatccggctatgggccctgcagctgatcttcgtgtccacgccagcgctcctagtggccatgcacgtggcctaccggagacatgagaagaagaggaagttcatcaagggggagataaagagtgaatttaaggacatcgaggagatcaaaacccagaaggtccgcatcgaaggctccctgtggtggacctacacaagcagcatcttcttccgggtcatcttcgaagccgccttcatgtacgtcttctatgtcatgtacgacggcttctccatgcagcggctggtgaagtgcaacgcctggccttgtcccaacactgtggactgctttgtgtcccggcccacggagaagactgtcttcacagtgttcatgattgcagtgtctggaatttgcatcctgctgaatgtcactgaattgtgttatttgctaattagatattgttctgggaagtcaaaaaagccagtttaacgcattgcccagttgttagattaagaaatagacagcatgagagggatgaggcaacccgtgctcagctgtcaaggctcagtcgctagcatttcccaacacaaagattctgaccttaaatgcaaccatttgaaacccctgtaggcctcaggtgaaactccagatgccacaatggagctctgctcccctaaagcctcaaaacaaaggcctaattctatgcctgtcttaattttctttcacttaagttagttccactgagaccccaggctgttaggggttattggtgtaaggtactttcatattttaaacagaggatatcggcatttgtttctttctctgaggacaagagaaaaaagccaggttccacagaggacacagagaaggtttgggtgtcctcctggggttctttttgccaactttccccacgttaaaggtgaacattggttctttcatttgctttggaagttttaatctctaacagtggacaaagttaccagtgccttaaactctgttacactttttggaagtgaaaactttgtagtatgataggttattttgatgtaaagatgttctggataccattatatgttccccctgtttcagaggctcagattgtaatatgtaaatggtatgtcattcgctactatgatttaatttgaaatatggtcttttggttatgaatactttgcagcacagctgagaggctgtctgttgtattcattgtggtcatagcacctaacaacattgtagcctcaatcgagtgagacagactagaagttcctagtgatggcttatgatagcaaatggcctcatgtcaaatatttagatgtaattttgtgtaagaaatacagactggatgtaccaccaactactacctgtaatgacaggcctgtccaacacatctcccttttccatgactgtggtagccagcatcggaaagaacgctgatttaaagaggtcgcttgggaattttattgacacagtaccatttaatggggaggacaaaatggggcaggggagggagaagtttctgtcgttaaaaacagatttggaaagactggactctaaagtctgttgattaaagatgagctttgtctacttcaaaagtttgtttgcttaccccttcagcctccaattttttaagtgaaaatatagctaataacatgtgaaaagaatagaagctaaggtttagataaatattgagcagatctataggaagattgaacctgaatattgccattatgcttgacatggtttccaaaaaatggtactccacatatttcagtgagggtaagtattttcctgttgtcaagaatagcattgtaaaagcattttgtaataataaagaatagctttaatgatatgcttgtaactaaaataattttgtaatgtatcaaatacatttaaaacattaaaatataatctctataataatttaaaatctaatatggttttaatagaacagcaaattttaatttcatctatcactttttatataaatacattaatgttttatatttcataacaccaatgggtaagttgccagagtgtctgaccccattctgccccagttacagaaaagcttctgtcaccagaaagtttggtggggaaggaagggaggaagatgatttctacctaaccccgtgcccacctctaccaggtttttgaggcatatcagtctatggacaatgtggtgtttggtctggaaacgtaccttggtgaatgctgagttggctggacatgacccgtttagctcctggatgaatcccagaagtggaccttcaaaatgttactcatagcatgaccttggctcactgcaacctctgcctcccaggctcaagcgatcctcccacctcagcgtcccaagtagctgggaccactggagtgtgccaccacactccactaattttttcattttttgtagaaacgaggtcccactatattgcccagtctggtctcgaactcctgggctgaagggatccccctgcctcagtctcctaaagtgcaaggattacaggcatgggccaccgcacctggcctgaaactgctttttattcctcagtgcccacttccatgggaaataagcctgccaggtcagcctgtccccatgggagtgactgcctgctacccccacaggcttgcccggccctcgtgagcctctcccagagacaccaccaacagttctgttctttcatggtacaagatttccatccaaggatttcaaagcatttcacacatcaataattagaagtattttcatagaggaccatacacttttaaaatggatttcaaagaacaaaaaccagtcaactatcacccaggtaatagaaaatgggaaatggtttctacctgacttccaaaatgctctgcacatagactgtgaaaataggattttttaagctgggtgcagaggcttatacctataatcccaacactttgggaggctgagacgagaggatcacttgagcccaggagttcaaaaccagcctgggcaatatagggagacattgtttctataaaaaataaaaatgttagccaggcaggcgtggtaacatgtgcctgtagtctcagctactcaggaggctgaggtgggaagattgcttgaacctgggaggtccatgctgcagtgagctgagattgtgccactgcactccagcctaggcgacagcaagatcctgtcccaaacaacaacaacatcaaaaaacacagaacttttaaaataagtacattcacttctacaagctatgtagattattactctcaagctattaaaagaccaagccaaaataattatgggctactctcgaccacttgtaggaatggatagagaggtctggtcacatgcctggaaattagagcttgagctctgaaaatgataatcctgactatatctcaaagcatcagtctgcactttgtatggagcaagaaaaagccttgtggaagcggcctcccacccagccgagccctcggcgtggacaagctctgctttttatgagcagtgggtgcagcctcgctgctccctcctcctgtcaaaagacagtcacagctggggtgagcagatcgggcccacttgggaggccccaaggaatatgctgcaggggtcgggcctgagccacccccacgggttggtctttgacaactagagagcagctgagaggtgggtaaaagctcactcacttaccctgacctcagtgtcctcatcttaaaatgggtttcctgaatctttccccggcttagtggcaatgaaataagataatttatgtaaacgttctccacatagtaaagcactaagtaacatatgactgtcatctgttttccactagacagatcccaacctggaagagtgacagatggtatttcagatacaagtgactcaagcaaagcttgataaactgggggctggaaaaaaatgcacatttacacaaagcctggagtaactgc

SEQ ID NO: 7 - protein, 226 aa, Artificial Sequence
  Exemplary Human Connexin 26 Protein Sequence
MDWGTLQTILGGVNKHSTSIGKIWLTVLFIFRIMILVVAAKEVWGDEQADFVCNTLQPGCKNVCYDHYFPISHIRLWALQLIFVSTPALLVAMHVAYRRHEKKRKFIKGEIKSEFKDIEEIKTQKVRIEGSLWWTYTSSIFFRVIFEAAFMYVFYVMYDGFSMQRLVKCNAWPCPNTVDCFVSRPTEKTVFTVFMIAVSGICILLNVTELCYLLIRYCSGKSKKPV

SEQ ID NO: 8 - DNA, 145 bp, Artificial Sequence
  Exemplary 5′ AAV ITR
ttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 9 - DNA, 145 bp, Artificial Sequence
  Exemplary 3′ AAV ITR
aggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 10 - DNA, 277 bp, Artificial Sequence
  Exemplary CBA promoter
gtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcg

SEQ ID NO: 11 - DNA, 279 bp, Artificial Sequence
  Exemplary CBA promoter
gtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcg

SEQ ID NO: 12 - DNA, 658 bp, Artificial Sequence
  Exemplary CMV/CBA enhancer/promoter
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcg

SEQ ID NO: 13 - DNA, 660 bp, Artificial Sequence
  Exemplary CMV/CBA enhancer/promoter
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcg

SEQ ID NO: 14 - DNA, 1671 bp, Artificial Sequence
  Exemplary CAG enhancer/promoter
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacag

SEQ ID NO: 15 - DNA, 1673 bp, Artificial Sequence
  Exemplary CAG enhancer/promoter
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacag

SEQ ID NO: 16 - DNA, 1725 bp, Artificial Sequence
  Exemplary Human ATOH1 enhancer-promoter
ctatggagtttgcataacaaacgtttggcagctcgctctcttacactccattaacaagctgtaacatatagctgcaggttgctataatctcattaatattttggaaacttgaatattgagtatttctgagtgctcattccccatatgccagccacttctgccatgctgactggttcctttctctccattattagcaattagcttcttaccttccaaagtcagatccaaggtatccaagatactagcaaaggaatcaactatgtgtgcaagttaagcatgcttaatatcacccaaacaaacaaagaggcagcatttcttaaagtaatgaagatagataaatcgggttagtcctttgcgacactgctggtgctttctagagttttatatattttaagcagcttgctttatattctgtctttgcctcccaccccaccagcacttttatttgtggagggttttggctcgccacactttgggaaacttatttgatttcacggagagctgaaggaagatcatttttggcaacagacaagtttaaacacgatttctatgggacattgctaactggggcccctaaggagaaaggggaaactgagcggagaatgggttaaatccttggaagcaggggagaggcaggggaggagagaagtcggaggagtataaagaaaaggacaggaaccaagaagcgtgggggtggtttgccgtaatgtgagtgtttcttaattagagaacggttgacaatagagggtctggcagaggctcctggccgcggtgcggagcgtctggagcggagcacgcgctgtcagctggtgagcgcactctcctttcaggcagctccccggggagctgtgcggccacatttaacaccatcatcacccctccccggcctcctcaacctcggcctcctcctcgtcgacagccttccttggcccccaccagcagagctcacagtagcgagcgtctctcgccgtctcccgcactcggccggggcctctctcctcccccagctgcgcagcgggagccgccactgcccactgcacctcccagcaaccagcccagcacgcaaagaagctgcgcaaagttaaagccaagcaatgccaaggggaggggaagctggaggcgggctttgagtggcttctgggcgcctggcgggtccagaatcgcccagagccgcccgcggtcgtgcacatctgacccgagtcagcttgggcaccagccgagagccggctccgcaccgctcccgcaccccagccgccggggtggtgacacacaccggagtcgaattacagccctgcaattaacatatgaatctgacgaatttaaaagaaggaaaaaaaaaaaaaaacctgagcaggcttgggagtcctctgcacacaagaacttttctcggggtgtaaaaactctttgattggctgctcgcacgcgcctgcccgcgccctccattggctgagaagacacgcgaccggcgcgaggagggggttgggagaggagcggggggagactgagtggcgcgtgccgctttttaaaggggcgcagcgccttcagcaaccggagaagcatagttgcacgcgacctggtgtgtgatctccgagtgggtgggggagggtcgaggagggaaaaaaaaataagacgttgcagaagagacccggaaagggccttttttttggttgagctggtgtcccagtgctgcctccgatcctgagcctccgagcctttgcagtgcaa

SEQ ID NO: 17 - DNA, 1553 bp, Artificial Sequence
  Exemplary Human GJB2 enhancer-promoter
aagcttcggtgaatttaaaacgtttggtggcagtgggtcaagtagccaggcggctgcgctagagtaccccgaagggacatcggcgacaccacaaacctcgcgctggcggctcgcccgcgcctttttcccctcccgcgcgcgcccggccccactcgcaccccgggcggtgccatcgcgtccacttccccggccgccccattccagctccggagctcggccgcagaaacgcccgctccagaaggcggcccccgccccccggcccaaggacgtgtgttggtccagccccccggttccccgagacccacgcggccgggcaaccgctctgggtctcgcggtccctccccgcgccaggttcctggccgggcagtccggggccggcgggctcacctgcgtcgggaggaagcgcggcggggccggggcgggggtctcggcgttggggtctctgcgctggggctcctgcgctcctaggcgggtcctgggccgggcgccgccgaggggctccgagtcggggagaggagcgcgcgggcgctgcggggccgcaacacctgtctcccgccgtggcgccttttaaccgcaccccacaccccgcctcttccctcggagactgggaaagttacggagggggcggcgccgcgggcggagcgcgcccggcctctgggtcctcagagcttcccgggtccgcgaacccccgaccgcccccgaaagccccgaaccccccaagtccccttcgaggtcccgatctcctagttcctttgagcccccatgagttccccaagtgcccccagcgccctgagtctcccccggttaccccgagcgccgcctcccccagccccttggcggcccgggtgaagcgggggcggctgagagtcgggaccccccaggaagcggcgccccagaccccggctccggcgctgtgccgtgggcggggttcagggatggctgtggtcgttgtcctctgtactccgcatagtgcgagaggacttggcatttatgagcgcttctttaattttttattgttagagaaacaggcattcctccaaggactgaagatctgttcgagtcgcggaggctgcgcgggcccgcgaggctctcgcagggggacctaggctgggtggcggggcagtgccctctggaatgggggttaacggtggccgaggagggggcgccgctggtgccggcgaagtccccgcttctttctcccctcaaaatctcaccaatccgaacgaacgccttctcgaatttccgattttattcaattactttcaacaatgtgccaaggactaaggttgggggcggtgggagagacaagcctcgtttttgccatggccggcaggggggtcccgccatctgcggagggtgccccccgcggcccccggcccagccaacttcctcctcttttcgcaactggggaactgcaaggaggtgactcctttcggggtgaggaggcccagacttttcagaaaggaaagagggcaggtaaaacctgccaagccccttcctgctcgatgcacacagcacgaaagggggaaactgataggattctgcggaagctt

SEQ ID NO: 18 - DNA, 381 bp, Artificial Sequence
  Exemplary CMV enhancer
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgg

SEQ ID NO: 19 - DNA, 1013 bp, Artificial Sequence
  Exemplary SV-40 synthetic intron
ggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacag

SEQ ID NO: 20 - DNA, 178 bp, Artificial Sequence
  Exemplary 5′ UTR Sequence
gttgcggccccgcagcgcccgcgcgctcctctccccgactcggagcccctcggcggcgcccggcccaggacccgcctaggagcgcaggagccccagcgcagagaccccaacgccgagacccccgccccggccccgccgcgcttcctcccgacgcagagcaaaccgcccagagtagaag

SEQ ID NO: 21 - DNA, 206 bp, Artificial Sequence
  Exemplary 5′ UTR Sequence
tttaggacccttgttcgcgaagaggtggtgtgcggctgagacccgcgtcctcaggacggttccatcagtgcctcgatcctgccccactggaggaggaaggcagcccgaacagcgctcacctaactaacagctgctgagagctgggttccgtggccatgcacctgggactgccttgagaagcgtgagcaaaccgcccagagtagaag

SEQ ID NO: 22 - DNA, 1432 bp, Artificial Sequence
  Exemplary 3′ UTR Sequence
cgcattgcccagttgttagattaagaaatagacagcatgagagggatgaggcaacccgtgctcagctgtcaaggctcagtcgctagcatttcccaacacaaagattctgaccttaaatgcaaccatttgaaacccctgtaggcctcaggtgaaactccagatgccacaatggagcctctgctcccctaaagcctcaaaacaaaggcctaattctatgcctgtcttaattttctttcacttaagttagttccactgagaccccaggctgttaggggttattggtgtaaggtactttcatattttaaacagaggatatcggcatttgtttctttctctgaggacaagagaaaaaagccaggttccacagaggacacagagaaggtttgggtgtcctcctggggttctttttgccaactttccccacgttaaaggtgaacattggttctttcatttgctttggaagttttaatctctaacagtggacaaagttaccagtgccttaaactctgttacactttttggaagtgaaaactttgtagtatgataggttattttgatgtaaagatgttctggataccattatatgttccccctgtttcagaggctcagattgtaatatgtaaatggtatgtcattcgctactatgatttaatttgaaatatggtcttttggttatgaatactttgcagcacagctgagaggctgtctgttgtattcattgtggtcatagcacctaacaacattgtagcctcaatcgagtgagacagactagaagttcctagtgatggcttatgatagcaaatggcctcatgtcaaatatttagatgtaattttgtgtaagaaatacagactggatgtaccaccaactactacctgtaatgacaggcctgtccaacacatctcccttttccatgactgtggtagccagcatcggaaagaacgctgatttaaagaggtcgcttgggaattttattgacacagtaccatttaatggggaggacaaaatggggcaggggagggagaagtttctgtcgttaaaaacagatttggaaagactggactctaaagtctgttgattaaagatgagctttgtctacttcaaaagtttgtttgcttaccccttcagcctccaattttttaagtgaaaatatagctaataacatgtgaaaagaatagaagctaaggtttagataaatattgagcagatctataggaagattgaacctgaatattgccattatgcttgacatggtttccaaaaaatggtactccacatatttcagtgagggtaagtattttcctgttgtcaagaatagcattgtaaaagcattttgtaataataaagaatagctttaatgatatgcttgtaactaaaataattttgtaatgtatcaaatacatttaaaacattaaaatataatctctataataa

SEQ ID NO: 23 - DNA, 82 bp, Artificial Sequence
  Exemplary splice donor intron
gtaagtatcaaggttacaagacaggtttaaggagaccaatagaaactgggcttgtcgagacagagaagactcttgcgtttct

SEQ ID NO: 24 - DNA, 51 bp, Artificial Sequence
  Exemplary splice acceptor intron
gataggcacctattggtcttactgacatccactttgcctttctctccacag

SEQ ID NO: 25 - DNA, 225 bp, Artificial Sequence
  Exemplary bGH poly(A) signal sequence
ctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgg

SEQ ID NO: 26 - DNA, 122 bp, Artificial Sequence
  Exemplary SV40 poly(A) signal sequence
aacttgtttattgcagcttataatggttacaaataaagcaatagcatcacaaatttcacaaataaagcatttttttcactgcattctagttgtggtttgtccaaactcatcaatgtatctta

SEQ ID NO: 27 - DNA, 1044 bp, Artificial Sequence
  Exemplary Regulatory sequence C3
cttcttctggagtcttttctggaataattctgggagtgggctcagcctgcgggagagtaacatttttataacttgatagatgtagctgagatgcctcccagaggggagacccgcctctcctccggcagctgtgcacgtaggcttgttcccagcagcctggccagggtggtccacctggtgtttctcatcttctttccccggagcgctgactcctgcgcgtcctcttggaagactcttgacaggacgggtgttttatgggtgtgattcagtgtcctcttgcatcagttcaatgtggtggtgttcaatcaacccttgtagcgttagcaaaatttgctcaagtcattccgcaggaatgtctgtgtcttgcttccaagaaagcttgtaagtgccggcaacaggccaagcagctcacaaacctgaccacaagcctgtgagtaattgtggggcagcacttagcagtcttttattttcgacttattaaagtctcatcttggcctcaccttctccctggaaggtggcgtgggtgggaaccactgggtcagatctttttcacccttgccgtggagccagtttcctgttgcatgtgggggaagcaacatgtggtgaagagtatagaaaacgaaaacatgtgggtacagtatgtataagtggagggaacaaactcataattccaactagtttctcatgagagactcatgaatcattgtggtagttctcaatataaacttaatctaggccggatgtggtggctcacacctgtaatctcagcactctgggtggatcacttgaggtcaggagtttgagaccagtctgaccaacatggagaaaccccatcgctactaaaaatacaaaattatccagatgtggtggctcacacctgtaatcccagcactttgggaggctgaggcgggtggatcacttgaggtcaggagtttgagaccagcctgaccaacatggagaaactgtgtctctactaaaaatacaaaattagctgggcgtggtgacgcatgcctgtaatcccagctatttggaggccgaagcagg

SEQ ID NO: 28 - DNA, 1044 bp, Artificial Sequence; identical to SEQ ID NO: 27
  Exemplary Regulatory sequence D7

SEQ ID NO: 29 - DNA, 22 bp, Artificial Sequence
  Exemplary cloning site A
ttgtcgacgcggccgcacgcgt

SEQ ID NO: 30 - DNA, 39 bp, Artificial Sequence
  Exemplary cloning site B
ctcctgggcaacgtgctggttattgtgaccggtgccacc

SEQ ID NO: 31 - DNA, 24 bp, Artificial Sequence
  Exemplary cloning site C
taagagctcgctgatcagcctcga

SEQ ID NO: 32 - DNA, 38 bp, Artificial Sequence
  Exemplary cloning site D
aagcttgaattcagctgacgtgcctcggaccgcctagg

SEQ ID NO: 33 - DNA, 9 bp, Artificial Sequence
  Exemplary cloning site E
taagagctc

SEQ ID NO: 34 - DNA, 15 bp, Artificial Sequence
  Exemplary cloning site F
gctgatcagcctcga

SEQ ID NO: 35 - DNA, 64 bp, Artificial Sequence
  Exemplary cloning site G
ggcattccggtactgttggtaaagccaccagcaaaccgcccagagtagaagaccggtggccacc

SEQ ID NO: 36 - DNA, 12 bp, Artificial Sequence
  Exemplary cloning site H
aagcttgaattc

SEQ ID NO: 37 - DNA, 26 bp, Artificial Sequence
  Exemplary cloning site I
agctgacgtgcctcggaccgcctagg

SEQ ID NO: 38 - protein, 159 aa, Artificial Sequence
  Exemplary DHFR destabilizing amino acid sequence
MISLIAALAVDYVIGMENAMPWNLPADLAWFKRNTLNKPVIMGRHTWESIGRPLPGRKNIILSSQPSTDDRVTWVKSVDEAIAACGDVPEIMVIGGGRVIEQFLPKAQKLYLTHIDAEVEGDTHFPDYEPDDWESVFSEFHDADAQNSHSYCFEILERR

SEQ ID NO: 39 - DNA, 483 bp, Artificial Sequence
  Exemplary DHFR destabilizing nucleotide sequence
ggtaccatcagtctgattgcggcgttagcggtagattacgttatcggcatggaaaacgccatgccgtggaacctgcctgccgatctcgcctggtttaaacgcaacaccttaaataaacccgtgattatgggccgccatacctgggaatcaatcggtcgtccgttgccaggacgcaaaaatattatcctcagcagtcaaccgagtacggacgatcgcgtaacgtgggtgaagtcggtggatgaagccatcgcggcgtgtggtgacgtaccagaaatcatggtgattggcggcggtcgcgttattgaacagttcttgccaaaagcgcaaaaactgtatctgacgcatatcgacgcagaagtggaaggcgacacccatttcccggattacgagccggatgactgggaatcggtattcagcgaattccacgatgctgatgcgcagaactctcacagctattgctttgagattctggagcggcgataa

SEQ ID NO: 40 - DNA, 474 bp, Artificial Sequence
  Exemplary destabilizing domain
atcagtctgattgcggcgttagcggtagattacgttatcggcatggaaaacgccatgccgtggaacctgcctgccgatctcgcctggtttaaacgcaacaccttaaataaacccgtgattatgggccgccatacctgggaatcaatcggtcgtccgttgccaggacgcaaaaatattatcctcagcagtcaaccgagtacggacgatcgcgtaacgtgggtgaagtcggtggatgaagccatcgcggcgtgtggtgacgtaccagaaatcatggtgattggcggcggtcgcgttattgaacagttcttgccaaaagcgcaaaaactgtatctgacgcatatcgacgcagaagtggaaggcgacacccatttcccggattacgagccggatgactgggaatcggtattcagcgaattccacgatgctgatgcgcagaactctcacagctattgctttgagattctggagcggcga

SEQ ID NO: 41 - protein, 112 aa, Artificial Sequence
  Exemplary FKBP12 destabilizing peptide amino acid sequence
MGVEKQVIRPGNGPKPAPGQTVTVHCTGFGKDGDLSQKFWSTKDEGQKPFSFQIGKGAVIKGWDEGVIGMQIGEVARLRCSSDYAYGAGGFPAWGIQPNSVLDFEIEVLSVQ

SEQ ID NO: 42 - DNA, 78 bp, Artificial Sequence
  Exemplary 3xFLAG tag sequence
ggatcccgggctgactacaaagaccatgacggtgattataaagatcatgacatcgactacaaggatgacgatgacaag

SEQ ID NO: 43 - DNA, 2208 bp, Artificial Sequence
  Exemplary AAV Anc80 Capsid DNA Sequence
atggctgccgatggttatcttccagattggctcgaggacaacctctctgagggcattcgcgagtggtgggacttgaaacctggagccccgaaacccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctcaaagcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaagagaccggtagagcaatcaccccaggaaccagactcctcttcgggcatcggcaagaaaggccagcagcccgcgaagaagagactcaactttgggcagacaggcgactcagagtcagtgcccgaccctcaaccactcggagaaccccccgcagccccctctggtgtgggatctaatacaatggcagcaggcggtggcgctccaatggcagacaataacgaaggcgccgacggagtgggtaacgcctcaggaaattggcattgcgattccacatggctgggcgacagagtcatcaccaccagcacccgaacctgggccctccccacctacaacaaccacctctacaagcaaatctccagccaatcgggagcaagcaccaacgacaacacctacttcggctacagcaccccctgggggtattttgactttaacagattccactgccacttctcaccacgtgactggcagcgactcatcaacaacaactggggattccggcccaagagactcaacttcaagctcttcaacatccaggtcaaggaggtcacgacgaatgatggcaccacgaccatcgccaataaccttaccagcacggttcaggtctttacggactcggaataccagctcccgtacgtcctcggctctgcgcaccagggctgcctgcctccgttcccggcggacgtcttcatgattcctcagtacgggtacctgactctgaacaatggcagtcaggccgtgggccgttcctccttctactgcctggagtactttccttctcaaatgctgagaacgggcaacaactttgagttcagctacacgtttgaggacgtgccttttcacagcagctacgcgcacagccaaagcctggaccggctgatgaaccccctcatcgaccagtacctgtactacctgtctcggactcagaccacgagtggtaccgcaggaaatcggacgttgcaattttctcaggccgggcctagtagcatggcgaatcaggccaaaaactggctacccgggccctgctaccggcagcaacgcgtctccaagacagcgaatcaaaataacaacagcaactttgcctggaccggtgccaccaagtatcatctgaatggcagagactctctggtaaatcccggtcccgctatggcaacccacaaggacgacgaagacaaattttttccgatgagcggagtcttaatatttgggaaacagggagctggaaatagcaacgtggaccttgacaacgttatgataaccagtgaggaagaaattaaaaccaccaacccagtggccacagaacagtacggcacggtggccactaacctgcaatcgtcaaacaccgctcctgctacagggaccgtcaacagtcaaggagccttacctggcatggtctggcagaaccgggacgtgtacctgcagggtcctatctgggccaagattcctcacacggacggacactttcatccctcgccgctgatgggaggctttggactgaaacacccgcctcctcagatcctgattaagaatacacctgttcccgcgaatcctccaactaccttcagtccagctaagtttgcgtcgttcatcacgcagtacagcaccggacaggtcagcgtggaaattgaatgggagctgcagaaagaaaacagcaaacgctggaacccagagattcaatacacttccaactacaacaaatctacaaatgtggactttgctgttgacacaaatggcgtttattctgagcctcgccccatcggcacccgttacctcacccgtaatctg

SEQ ID NO: 44 - protein, 736 aa, Artificial Sequence
  Exemplary AAV Anc80 Capsid Amino Acid Sequence
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEQSPQEPDSSSGIGKKGQQPAKKRLNFGQTGDSESVPDPQPLGEPPAAPSGVGSNTMAAGGGAPMADNNEGADGVGNASGNWHCDSTWLGDRVITTSTRTWALPTYNNHLYKQISSQSGASTNDNTYFGYSTPWGYFDFNRFHCHFSPRDWQRLINNNWGFRPKRLNFKLFNIQVKEVTTNDGTTTIANNLTSTVQVFTDSEYQLPYVLGSAHQGCLPPFPADVFMIPQYGYLTLNNGSQAVGRSSFYCLEYFPSQMLRTGNNFEFSYTFEDVPFHSSYAHSQSLDRLMNPLIDQYLYYLSRTQTTSGTAGNRTLQFSQAGPSSMANQAKNWLPGPCYRQQRVSKTANQNNNSNFAWTGATKYHLNGRDSLVNPGPAMATHKDDEDKFFPMSGVLIFGKQGAGNSNVDLDNVMITSEEEIKTTNPVATEQYGTVATNLQSSNTAPATGTVNSQGALPGMVWQNRDVYLQGPIWAKIPHTDGHFHPSPLMGGFGLKHPPPQILIKNTPVPANPPTTFSPAKFASFITQYSTGQVSVEIEWELQKENSKRWNPEIQYTSNYNKSTNVDFAVDTNGVYSEPRPIGTRYLTRNL

SEQ ID NO: 45 - DNA, 2989 bp, Artificial Sequence
  Exemplary Construct sequence
ttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcctttgtcgacgcggccgcacgcgtgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgaccggtgccaccatggattggggcacgctgcagacgatcctggggggtgtgaacaaacactccaccagcattggaaagatctggctcaccgtcctcttcatttttcgcattatgatcctcgttgtggctgcaaaggaggtgtggggagatgagcaggccgactttgtctgcaacaccctgcagccaggctgcaagaacgtgtgctacgatcactacttccccatctcccacatccggctatgggccctgcagctgatcttcgtgtccacgccagcgctcctagtggccatgcacgtggcctaccggagacatgagaagaagaggaagttcatcaagggggagataaagagtgaatttaaggacatcgaggagatcaaaacccagaaggtccgcatcgaaggctccctgtggtggacctacacaagcagcatcttcttccgggtcatcttcgaagccgccttcatgtacgtcttctatgtcatgtacgacggcttctccatgcagcggctggtgaagtgcaacgcctggccttgtcccaacactgtggactgctttgtgtcccggcccacggagaagactgtcttcacagtgttcatgattgcagtgtctggaatttgcatcctgctgaatgtcactgaattgtgttatttgctaattagatattgttctgggaagtcaaaaaagccagtttaagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggaagcttgaattcagctgacgtgcctcggaccgcctaggaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 46 - DNA, 4421 bp, Artificial Sequence
  Exemplary Construct sequence
ttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcctttgtcgacgcggccgcacgcgtgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgaccggtgccaccatggattggggcacgctgcagacgatcctggggggtgtgaacaaacactccaccagcattggaaagatctggctcaccgtcctcttcatttttcgcattatgatcctcgttgtggctgcaaaggaggtgtggggagatgagcaggccgactttgtctgcaacaccctgcagccaggctgcaagaacgtgtgctacgatcactacttccccatctcccacatccggctatgggccctgcagctgatcttcgtgtccacgccagcgctcctagtggccatgcacgtggcctaccggagacatgagaagaagaggaagttcatcaagggggagataaagagtgaatttaaggacatcgaggagatcaaaacccagaaggtccgcatcgaaggctccctgtggtggacctacacaagcagcatcttcttccgggtcatcttcgaagccgccttcatgtacgtcttctatgtcatgtacgacggcttctccatgcagcggctggtgaagtgcaacgcctggccttgtcccaacactgtggactgctttgtgtcccggcccacggagaagactgtcttcacagtgttcatgattgcagtgtctggaatttgcatcctgctgaatgtcactgaattgtgttatttgctaattagatattgttctgggaagtcaaaaaagccagtttaagagctccgcattgcccagttgttagattaagaaatagacagcatgagagggatgaggcaacccgtgctcagctgtcaaggctcagtcgctagcatttcccaacacaaagattctgaccttaaatgcaaccatttgaaacccctgtaggcctcaggtgaaactccagatgccacaatggagcctctgctcccctaaagcctcaaaacaaaggcctaattctatgcctgtcttaattttctttcacttaagttagttccactgagaccccaggctgttaggggttattggtgtaaggtactttcatattttaaacagaggatatcggcatttgtttctttctctgaggacaagagaaaaaagccaggttccacagaggacacagagaaggtttgggtgtcctcctggggttctttttgccaactttccccacgttaaaggtgaacattggttctttcatttgctttggaagttttaatctctaacagtggacaaagttaccagtgccttaaactctgttacactttttggaagtgaaaactttgtagtatgataggttattttgatgtaaagatgttctggataccattatatgttccccctgtttcagaggctcagattgtaatatgtaaatggtatgtcattcgctactatgatttaatttgaaatatggtcttttggttatgaatactttgcagcacagctgagaggctgtctgttgtattcattgtggtcatagcacctaacaacattgtagcctcaatcgagtgagacagactagaagttcctagtgatggcttatgatagcaaatggcctcatgtcaaatatttagatgtaattttgtgtaagaaatacagactggatgtaccaccaactactacctgtaatgacaggcctgtccaacacatctcccttttccatgactgtggtagccagcatcggaaagaacgctgatttaaagaggtcgcttgggaattttattgacacagtaccatttaatggggaggacaaaatggggcaggggagggagaagtttctgtcgttaaaaacagatttggaaagactggactctaaagtctgttgattaaagatgagctttgtctacttcaaaagtttgtttgcttaccccttcagcctccaattttttaagtgaaaatatagctaataacatgtgaaaagaatagaagctaaggtttagataaatattgagcagatctataggaagattgaacctgaatattgccattatgcttgacatggtttccaaaaaatggtactccacatatttcagtgagggtaagtattttcctgttgtcaagaatagcattgtaaaagcattttgtaataataaagaatagctttaatgatatgcttgtaactaaaataattttgtaatgtatcaaatacatttaaaacattaaaatataatctctataataagctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggaagcttgaattcagctgacgtgcctcggaccgcctaggaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 47 - DNA, 3938 bp, Artificial Sequence
  Exemplary Construct sequence
ttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcctttgtcgacgcggccgcacgcgtaagcttcggtgaatttaaaacgtttggtggcagtgggtcaagtagccaggcggctgcgctagagtaccccgaagggacatcggcgacaccacaaacctcgcgctggcggctcgcccgcgcctttttcccctcccgcgcgcgcccggccccactcgcaccccgggcggtgccatcgcgtccacttccccggccgccccattccagctccggagctcggccgcagaaacgcccgctccagaaggcggcccccgccccccggcccaaggacgtgtgttggtccagccccccggttccccgagacccacgcggccgggcaaccgctctgggtctcgcggtccctccccgcgccaggttcctggccgggcagtccggggccggcgggctcacctgcgtcgggaggaagcgcggcggggccggggcgggggtctcggcgttggggtctctgcgctggggctcctgcgctcctaggcgggtcctgggccgggcgccgccgaggggctccgagtcggggagaggagcgcgcgggcgctgcggggccgcaacacctgtctcccgccgtggcgccttttaaccgcaccccacaccccgcctcttccctcggagactgggaaagttacggagggggcggcgccgcgggcggagcgcgcccggcctctgggtcctcagagcttcccgggtccgcgaacccccgaccgcccccgaaagccccgaaccccccaagtccccttcgaggtcccgatctcctagttcctttgagcccccatgagttccccaagtgcccccagcgccctgagtctcccccggttaccccgagcgccgcctcccccagccccttggcggcccgggtgaagcgggggcggctgagagtcgggaccccccaggaagcggcgccccagaccccggctccggcgctgtgccgtgggcggggttcagggatggctgtggtcgttgtcctctgtactccgcatagtgcgagaggacttggcatttatgagcgcttctttaattttttattgttagagaaacaggcattcctccaaggactgaagatctgttcgagtcgcggaggctgcgcgggcccgcgaggctctcgcagggggacctaggctgggtggcggggcagtgccctctggaatgggggttaacggtggccgaggagggggcgccgctggtgccggcgaagtccccgcttctttctcccctcaaaatctcaccaatccgaacgaacgccttctcgaatttccgattttattcaattactttcaacaatgtgccaaggactaaggttgggggcggtgggagagacaagcctcgtttttgccatggccggcaggggggtcccgccatctgcggagggtgccccccgcggcccccggcccagccaacttcctcctcttttcgcaactggggaactgcaaggaggtgactcctttcggggtgaggaggcccagacttttcagaaaggaaagagggcaggtaaaacctgccaagccccttcctgctcgatgcacacagcacgaaagggggaaactgataggattctgcggaagcttggcattccggtactgttggtaaagccaccagcaaaccgcccagagtagaagaccggtggccaccatggattggggcacgctgcagacgatcctggggggtgtgaacaaacactccaccagcattggaaagatctggctcaccgtcctcttcatttttcgcattatgatcctcgttgtggctgcaaaggaggtgtggggagatgagcaggccgactttgtctgcaacaccctgcagccaggctgcaagaacgtgtgctacgatcactacttccccatctcccacatccggctatgggccctgcagctgatcttcgtgtccacgccagcgctcctagtggccatgcacgtggcctaccggagacatgagaagaagaggaagttcatcaagggggagataaagagtgaatttaaggacatcgaggagatcaaaacccagaaggtccgcatcgaaggctccctgtggtggacctacacaagcagcatcttcttccgggtcatcttcgaagccgccttcatgtacgtcttctatgtcatgtacgacggcttctccatgcagcggctggtgaagtgcaacgcctggccttgtcccaacactgtggactgctttgtgtcccggcccacggagaagactgtcttcacagtgttcatgattgcagtgtctggaatttgcatcctgctgaatgtcactgaattgtgttatttgctaattagatattgttctgggaagtcaaaaaagccagtttaagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggaagcttgaattccttcttctggagtcttttctggaataattctgggagtgggctcagcctgcgggagagtaacatttttataacttgatagatgtagctgagatgcctcccagaggggagacccgcctctcctccggcagctgtgcacgtaggcttgttcccagcagcctggccagggtggtccacctggtgtttctcatcttctttccccggagcgctgactcctgcgcgtcctcttggaagactcttgacaggacgggtgttttatgggtgtgattcagtgtcctcttgcatcagttcaatgtggtggtgttcaatcaacccttgtagcgttagcaaaatttgctcaagtcattccgcaggaatgtctgtgtcttgcttccaagaaagcttgtaagtgccggcaacaggccaagcagctcacaaacctgaccacaagcctgtgagtaattgtggggcagcacttagcagtcttttattttcgacttattaaagtctcatcttggcctcaccttctccctggaaggtggcgtgggtgggaaccactgggtcagatctttttcacccttgccgtggagccagtttcctgttgcatgtgggggaagcaacatgtggtgaagagtatagaaaacgaaaacatgtgggtacagtatgtataagtggagggaacaaactcataattccaactagtttctcatgagagactcatgaatcattgtggtagttctcaatataaacttaatctaggccggatgtggtggctcacacctgtaatctcagcactctgggtggatcacttgaggtcaggagtttgagaccagtctgaccaacatggagaaaccccatcgctactaaaaatacaaaattatccagatgtggtggctcacacctgtaatcccagcactttgggaggctgaggcgggtggatcacttgaggtcaggagtttgagaccagcctgaccaacatggagaaactgtgtctctactaaaaatacaaaattagctgggcgtggtgacgcatgcctgtaatcccagctatttggaggccgaagcaggagctgacgtgcctcggaccgcctaggaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 48 - DNA, 3916 bp, Artificial Sequence
  Exemplary Construct sequence
ttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcctttgtcgacgcggccgcacgcgtaagcttcggtgaatttaaaacgtttggtggcagtgggtcaagtagccaggcggctgcgctagagtaccccgaagggacatcggcgacaccacaaacctcgcgctggcggctcgcccgcgcctttttcccctcccgcgcgcgcccggccccactcgcaccccgggcggtgccatcgcgtccacttccccggccgccccattccagctccggagctcggccgcagaaacgcccgctccagaaggcggcccccgccccccggcccaaggacgtgtgttggtccagccccccggttccccgagacccacgcggccgggcaaccgctctgggtctcgcggtccctccccgcgccaggttcctggccgggcagtccggggccggcgggctcacctgcgtcgggaggaagcgcggcggggccggggcgggggtctcggcgttggggtctctgcgctggggctcctgcgctcctaggcgggtcctgggccgggcgccgccgaggggctccgagtcggggagaggagcgcgcgggcgctgcggggccgcaacacctgtctcccgccgtggcgccttttaaccgcaccccacaccccgcctcttccctcggagactgggaaagttacggagggggcggcgccgcgggcggagcgcgcccggcctctgggtcctcagagcttcccgggtccgcgaacccccgaccgcccccgaaagccccgaaccccccaagtccccttcgaggtcccgatctcctagttcctttgagcccccatgagttccccaagtgcccccagcgccctgagtctcccccggttaccccgagcgccgcctcccccagccccttggcggcccgggtgaagcgggggcggctgagagtcgggaccccccaggaagcggcgccccagaccccggctccggcgctgtgccgtgggcggggttcagggatggctgtggtcgttgtcctctgtactccgcatagtgcgagaggacttggcatttatgagcgcttctttaattttttattgttagagaaacaggcattcctccaaggactgaagatctgttcgagtcgcggaggctgcgcgggcccgcgaggctctcgcagggggacctaggctgggtggcggggcagtgccctctggaatgggggttaacggtggccgaggagggggcgccgctggtgccggcgaagtccccgcttctttctcccctcaaaatctcaccaatccgaacgaacgccttctcgaatttccgattttattcaattactttcaacaatgtgccaaggactaaggttgggggcggtgggagagacaagcctcgtttttgccatggccggcaggggggtcccgccatctgcggagggtgccccccgcggcccccggcccagccaacttcctcctcttttcgcaactggggaactgcaaggaggtgactcctttcggggtgaggaggcccagacttttcagaaaggaaagagggcaggtaaaacctgccaagccccttcctgctcgatgcacacagcacgaaagggggaaactgataggattctgcggaagcttggcattccggtactgttggtaaagccaccagcaaaccgcccagagtagaagaccggtggccaccatggattggggcacgctgcagacgatcctggggggtgtgaacaaacactccaccagcattggaaagatctggctcaccgtcctcttcatttttcgcattatgatcctcgttgtggctgcaaaggaggtgtggggagatgagcaggccgactttgtctgcaacaccctgcagccaggctgcaagaacgtgtgctacgatcactacttccccatctcccacatccggctatgggccctgcagctgatcttcgtgtccacgccagcgctcctagtggccatgcacgtggcctaccggagacatgagaagaagaggaagttcatcaagggggagataaagagtgaatttaaggacatcgaggagatcaaaacccagaaggtccgcatcgaaggctccctgtggtggacctacacaagcagcatcttcttccgggtcatcttcgaagccgccttcatgtacgtcttctatgtcatgtacgacggcttctccatgcagcggctggtgaagtgcaacgcctggccttgtcccaacactgtggactgctttgtgtcccggcccacggagaagactgtcttcacagtgttcatgattgcagtgtctggaatttgcatcctgctgaatgtcactgaattgtgttatttgctaattagatattgttctgggaagtcaaaaaagccagtttaagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggaagcttgaattccctgtaaagccaattccaacccacttgtaattaagagaaaatcccacggttcctaattgaaagtcctttgttctatttcttgggtatttgtgttttaggccttatttttagatgcatcattaaagatttttaaagtcctttcaggcatcaggactgatgatgctgaatgatggagggttgtggataagtttttttgttttttttttaaccaggttaaaggctttcctgttatcctactatgcttaattaagagctgtatttcttaatatcattggtgcctgattagatttaacttttagatacagtctgtaagatttttgaaccagaaaaacctaaataacttatgactgttagcagtcatattctagaagaagcaaatgtactgaattcttatgtacctaggattttaagggagtacatacaaatctttcctcagtagcaggtactttatttttataacacacacatttaagctgagttaaatatgcagaactggttgtacttctttggcaggaaaagggaagcttaggatatcttgtgaccaactacctcttccttctcaaataactggcaaataacttcaggaaaatccagttatgttgtgtcatattgcaccccctaggaagtactggattcttagtcttgagtgacttttaaataaagctacctttttctctttcttacatcgcaagatcttcaaatgtaccattcccgcacagagagtccaaggtaaaaggactgaaaccaaactttgtttttgtaagtattttggtcagtgcaatgagttcagagaccaggaggttaatgattgtgaagtcttgtcaacagcaacaccgtgtatgacctgtggtgcttagatgttcagaaaccccaaggttaaaatgtccctgaccacatatcaggcaaaaggaatgtaaggaaaaccaacttaatccttttgtcaagaagtataaatgatgtatctttccaatcgggttgcattgacttttgggtccaaatagcttgtgtccacaggcatcttcagctgacgtgcctcggaccgcctaggaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 49 - DNA, 2894 bp, Artificial Sequence
  Exemplary Construct sequence
ttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcctttgtcgacgcggccgcacgcgtaagcttcggtgaatttaaaacgtttggtggcagtgggtcaagtagccaggcggctgcgctagagtaccccgaagggacatcggcgacaccacaaacctcgcgctggcggctcgcccgcgcctttttcccctcccgcgcgcgcccggccccactcgcaccccgggcggtgccatcgcgtccacttccccggccgccccattccagctccggagctcggccgcagaaacgcccgctccagaaggcggcccccgccccccggcccaaggacgtgtgttggtccagccccccggttccccgagacccacgcggccgggcaaccgctctgggtctcgcggtccctccccgcgccaggttcctggccgggcagtccggggccggcgggctcacctgcgtcgggaggaagcgcggcggggccggggcgggggtctcggcgttggggtctctgcgctggggctcctgcgctcctaggcgggtcctgggccgggcgccgccgaggggctccgagtcggggagaggagcgcgcgggcgctgcggggccgcaacacctgtctcccgccgtggcgccttttaaccgcaccccacaccccgcctcttccctcggagactgggaaagttacggagggggcggcgccgcgggcggagcgcgcccggcctctgggtcctcagagcttcccgggtccgcgaacccccgaccgcccccgaaagccccgaaccccccaagtccccttcgaggtcccgatctcctagttcctttgagcccccatgagttccccaagtgcccccagcgccctgagtctcccccggttaccccgagcgccgcctcccccagccccttggcggcccgggtgaagcgggggcggctgagagtcgggaccccccaggaagcggcgccccagaccccggctccggcgctgtgccgtgggcggggttcagggatggctgtggtcgttgtcctctgtactccgcatagtgcgagaggacttggcatttatgagcgcttctttaattttttattgttagagaaacaggcattcctccaaggactgaagatctgttcgagtcgcggaggctgcgcgggcccgcgaggctctcgcagggggacctaggctgggtggcggggcagtgccctctggaatgggggttaacggtggccgaggagggggcgccgctggtgccggcgaagtccccgcttctttctcccctcaaaatctcaccaatccgaacgaacgccttctcgaatttccgattttattcaattactttcaacaatgtgccaaggactaaggttgggggcggtgggagagacaagcctcgtttttgccatggccggcaggggggtcccgccatctgcggagggtgccccccgcggcccccggcccagccaacttcctcctcttttcgcaactggggaactgcaaggaggtgactcctttcggggtgaggaggcccagacttttcagaaaggaaagagggcaggtaaaacctgccaagccccttcctgctcgatgcacacagcacgaaagggggaaactgataggattctgcggaagcttggcattccggtactgttggtaaagccaccagcaaaccgcccagagtagaagaccggtggccaccatggattggggcacgctgcagacgatcctggggggtgtgaacaaacactccaccagcattggaaagatctggctcaccgtcctcttcatttttcgcattatgatcctcgttgtggctgcaaaggaggtgtggggagatgagcaggccgactttgtctgcaacaccctgcagccaggctgcaagaacgtgtgctacgatcactacttccccatctcccacatccggctatgggccctgcagctgatcttcgtgtccacgccagcgctcctagtggccatgcacgtggcctaccggagacatgagaagaagaggaagttcatcaagggggagataaagagtgaatttaaggacatcgaggagatcaaaacccagaaggtccgcatcgaaggctccctgtggtggacctacacaagcagcatcttcttccgggtcatcttcgaagccgccttcatgtacgtcttctatgtcatgtacgacggcttctccatgcagcggctggtgaagtgcaacgcctggccttgtcccaacactgtggactgctttgtgtcccggcccacggagaagactgtcttcacagtgttcatgattgcagtgtctggaatttgcatcctgctgaatgtcactgaattgtgttatttgctaattagatattgttctgggaagtcaaaaaagccagtttaagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggaagcttgaattcagctgacgtgcctcggaccgcctaggaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 50 - DNA, 2987 bp, Artificial Sequence
  Exemplary Construct sequence
ttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcctttgtcgacgcggccgcacgcgtgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgaccggtgccaccatggattggggcacgctgcagacgatcctggggggtgtgaacaaacactccaccagcattggaaagatctggctcaccgtcctcttcatttttcgcattatgatcctcgttgtggctgcaaaggaggtgtggggagatgagcaggccgactttgtctgcaacaccctgcagccaggctgcaagaacgtgtgctacgatcactacttccccatctcccacatccggctatgggccctgcagctgatcttcgtgtccacgccagcgctcctagtggccatgcacgtggcctaccggagacatgagaagaagaggaagttcatcaagggggagataaagagtgaatttaaggacatcgaggagatcaaaacccagaaggtccgcatcgaaggctccctgtggtggacctacacaagcagcatcttcttccgggtcatcttcgaagccgccttcatgtacgtcttctatgtcatgtacgacggcttctccatgcagcggctggtgaagtgcaacgcctggccttgtcccaacactgtggactgctttgtgtcccggcccacggagaagactgtcttcacagtgttcatgattgcagtgtctggaatttgcatcctgctgaatgtcactgaattgtgttatttgctaattagatattgttctgggaagtcaaaaaagccagtttaagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggaagcttgaattcagctgacgtgcctcggaccgcctaggaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 51 - DNA, 4419 bp, Artificial Sequence
  Exemplary Construct sequence
ttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcctttgtcgacgcggccgcacgcgtgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgaccggtgccaccatggattggggcacgctgcagacgatcctggggggtgtgaacaaacactccaccagcattggaaagatctggctcaccgtcctcttcatttttcgcattatgatcctcgttgtggctgcaaaggaggtgtggggagatgagcaggccgactttgtctgcaacaccctgcagccaggctgcaagaacgtgtgctacgatcactacttccccatctcccacatccggctatgggccctgcagctgatcttcgtgtccacgccagcgctcctagtggccatgcacgtggcctaccggagacatgagaagaagaggaagttcatcaagggggagataaagagtgaatttaaggacatcgaggagatcaaaacccagaaggtccgcatcgaaggctccctgtggtggacctacacaagcagcatcttcttccgggtcatcttcgaagccgccttcatgtacgtcttctatgtcatgtacgacggcttctccatgcagcggctggtgaagtgcaacgcctggccttgtcccaacactgtggactgctttgtgtcccggcccacggagaagactgtcttcacagtgttcatgattgcagtgtctggaatttgcatcctgctgaatgtcactgaattgtgttatttgctaattagatattgttctgggaagtcaaaaaagccagtttaagagctccgcattgcccagttgttagattaagaaatagacagcatgagagggatgaggcaacccgtgctcagctgtcaaggctcagtcgctagcatttcccaacacaaagattctgaccttaaatgcaaccatttgaaacccctgtaggcctcaggtgaaactccagatgccacaatggagcctctgctcccctaaagcctcaaaacaaaggcctaattctatgcctgtcttaattttctttcacttaagttagttccactgagaccccaggctgttaggggttattggtgtaaggtactttcatattttaaacagaggatatcggcatttgtttctttctctgaggacaagagaaaaaagccaggttccacagaggacacagagaaggtttgggtgtcctcctggggttctttttgccaactttccccacgttaaaggtgaacattggttctttcatttgctttggaagttttaatctctaacagtggacaaagttaccagtgccttaaactctgttacactttttggaagtgaaaactttgtagtatgataggttattttgatgtaaagatgttctggataccattatatgttccccctgtttcagaggctcagattgtaatatgtaaatggtatgtcattcgctactatgatttaatttgaaatatggtcttttggttatgaatactttgcagcacagctgagaggctgtctgttgtattcattgtggtcatagcacctaacaacattgtagcctcaatcgagtgagacagactagaagttcctagtgatggcttatgatagcaaatggcctcatgtcaaatatttagatgtaattttgtgtaagaaatacagactggatgtaccaccaactactacctgtaatgacaggcctgtccaacacatctcccttttccatgactgtggtagccagcatcggaaagaacgctgatttaaagaggtcgcttgggaattttattgacacagtaccatttaatggggaggacaaaatggggcaggggagggagaagtttctgtcgttaaaaacagatttggaaagactggactctaaagtctgttgattaaagatgagctttgtctacttcaaaagtttgtttgcttaccccttcagcctccaattttttaagtgaaaatatagctaataacatgtgaaaagaatagaagctaaggtttagataaatattgagcagatctataggaagattgaacctgaatattgccattatgcttgacatggtttccaaaaaatggtactccacatatttcagtgagggtaagtattttcctgttgtcaagaatagcattgtaaaagcattttgtaataataaagaatagctttaatgatatgcttgtaactaaaataattttgtaatgtatcaaatacatttaaaacattaaaatataatctctataataagctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggaagcttgaattcagctgacgtgcctcggaccgcctaggaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 52 - DNA, 119 bp, Artificial Sequence
  Exemplary 5′ AAV ITR
ctgcgcgctcgctcgctcactgaggccgcccgggcgtcgggcgacctttggtcgcccggcctcagtgagcgagcgagcgcgcagagagggagtggccaactccatcactaggggttcct

SEQ ID NO: 53 - DNA, 130 bp, Artificial Sequence
  Exemplary 3′ AAV ITR
aggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 54 - DNA, 900 bp, Artificial Sequence
  Exemplary Human SLC26A4 enhancer-promoter
cggaaggttgatgtacagaggtctgtattttggagcctcttctgtatttacttcagaacactaacaatcaggcgagaatgttctggtttatcaaacccttccttctgcctttcatcttaaccatgcattagttttaacaaagttcatcccaacagaagacaaaacactgatgaggtaggatagctccagctcctcctccctctcttctagtcttgatttccatgtagtccagtttattccttccctgattgtccaggagaatgagaaaaagaaaaaacagagtctagtgggtaagaaagggccacctggacggcttgatttggattgtgaaataaaacacacacacatgcacacgtagaataagtggctaaaatctgagtaaatcgtgaactctctgtatcctccacccattgaatactcctaaaagactttctagaaattcaaggacttattaatatagaaacctggccattgttcctcttctcctccccatgtggtatgagagcacctgtggcaggctcccagagaccacggacctcttcctctaggcgggctctgctcttctttaaggagtcccacagggcctggcccgcccctgacctcgcaacccttgagattagtaacgggatgagtgaggatccgggtggcccctgcgtggcagccagtaagagtctcagccttcccggttcgggaaaggggaagaatgcaggaggggtaggatttctttcctgataggatcggttgggaaagaccgcagcctgtgtgtgtctttcccttcgaccaaggtgtctgttgctccgtaaataaaacgtcccactgccttctgagagcgctataaaggcagcggaagggtagtccgcggggcattccgggcggggcgcgagcagagacaggtgagtt

SEQ ID NO: 55 - DNA, 1269 bp, Artificial Sequence
  Exemplary Human LGR5 enhancer-promoter
agggctatttgtacctcaacgagggcttctctccaagaaagccctgaatccttttcctcctttttcctgcagattcactataggacactttttgaagcaagagcatgcattttccccctggcgctctgcagcggttctcagagcccagtgtcactcacataggtgggactgctctcagttcagagagcgctgggacacttaagatgaaaagtccctggaagttagcaaacagccatctgtcactctggcatcgatttactaaaagtgacttctagggtattctaaaccacttttaaaaaacaaatgagtcacttcgacttcctcaccccgcaagagataggaaggcagcagtggagtgctcgctcaggagctgtatttgtttagcgattagcctagagctttgattttagggcaaaagcgagccagacagtgcggcagacgtaaggatcaaaaaggccacctatcattcgccggggacgcctgcctccttaccctgataacgtaactatttctctgcataggattttagtttttgtgtttttgttttgttttattctgtttaatcacttcaagtatctcatccattatttgaagcgggctcggaggaaacgtgccgcatcctccagtccttgtgcgtctgtttaggtctctccgaagcaggtccctctcgactcttagatctgggtctccagcacgcatgaaggggtaagggtgggggggtcccctattccggcgcgcggcgttgagcactgaatcttccaggcggaggctcagtgggagcgccgagaactcgccagtaccgcgcgctgcctgctgcctgctgcctcccagcccaggacttgggaaaggagggaggggacaagtggagggaaagtggggccgggcggggggtgcctgggaagccaggctgcgctgacgtcactgggcgcgcaattcgggctggagcgctttaaaaaacgagcgtgcaagcagagatgctgctccacaccgctcaggccgcgagcagcagcaaggcgcaccgccactgtcgccgctgcagccagggctgctccgaaggccggcgtggcggcaaccggcacctctgtccccgccgcgcttctcctcgccgcccacgccgtggggtcaggaacgcggcgtctggcgctgcagacgcccgctgagttgcagaagcccacggagcggcgcccggcgcgccacggcccgtagcagtccggtgctgctctccgcccgcgtccggctcgtggccccctacttcgggcaccgaccggt

SEQ ID NO: 56 - DNA, 463 bp, Artificial Sequence
  Exemplary Human SYN1 enhancer-promoter
tgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcagtcgagaa

SEQ ID NO: 57 - DNA, 2204 bp, Artificial Sequence
  Exemplary Human GFAP enhancer-promoter
cccacctccctctctgtgctgggactcacagagggagacctcaggaggcagtctgtccatcacatgtccaaatgcagagcataccctgggctgggcgcagtggcgcacaactgtaattccagcactttgggaggctgatgtggaaggatcacttgagcccagaagttctagaccagcctgggcaacatggcaagaccctatctctacaaaaaaagttaaaaaatcagccacgtgtggtgacacacacctgtagtcccagctattcaggaggctgaggtgaggggatcacttaaggctgggaggttgaggctgcagtgagtcgtggttgcgccactgcactccagcctgggcaacagtgagaccctgtctcaaaagacaaaaaaaaaaaaaaaaaaaaaaagaacatatcctggtgtggagtaggggacgctgctctgacagaggctcgggggcctgagctggctctgtgagctggggaggaggcagacagccaggccttgtctgcaagcagacctggcagcattgggctggccgccccccagggcctcctcttcatgcccagtgaatgactcaccttggcacagacacaatgttcggggtgggcacagtgcctgcttcccgccgcaccccagcccccctcaaatgccttccgagaagcccattgagcagggggcttgcattgcaccccagcctgacagcctggcatcttgggataaaagcagcacagccccctaggggctgcccttgctgtgtggcgccaccggcggtggagaacaaggctctattcagcctgtgcccaggaaaggggatcaggggatgcccaggcatggacagtgggtggcagggggggagaggagggctgtctgcttcccagaagtccaaggacacaaatgggtgaggggactgggcagggttctgaccctgtgggaccagagtggagggcgtagatggacctgaagtctccagggacaacagggcccaggtctcaggctcctagttgggcccagtggctccagcgtttccaaacccatccatccccagaggttcttcccatctctccaggctgatgtgtgggaactcgaggaaataaatctccagtgggagacggaggggtggccagggaaacggggcgctgcaggaataaagacgagccagcacagccagctcatgtgtaacggctttgtggagctgtcaaggcctggtctctgggagagaggcacagggaggccagacaaggaaggggtgacctggagggacagatccaggggctaaagtcctgataaggcaagagagtgccggccccctcttgccctatcaggacctccactgccacatagaggccatgattgacccttagacaaagggctggtgtccaatcccagcccccagccccagaactccagggaatgaatgggcagagagcaggaatgtgggacatctgtgttcaagggaaggactccaggagtctgctgggaatgaggcctagtaggaaatgaggtggcccttgagggtacagaacaggttcattcttcgccaaattcccagcaccttgcaggcacttacagctgagtgagataatgcctgggttatgaaatcaaaaagttggaaagcaggtcagaggtcatctggtacagcccttccttcccttttttttttttttttttgtgagacaaggtctctctctgttgcccaggctggagtggcgcaaacacagctcactgcagcctcaacctactgggctcaagcaatcctccagcctcagcctcccaaagtgctgggattacaagcatgagccaccccactcagccctttccttcctttttaattgatgcataataattgtaagtattcatcatggtccaaccaaccctttcttgacccaccttcctagagagagggtcctcttgcttcagcggtcagggccccagacccatggtctggctccaggtaccacctgcctcatgcaggagttggcgtgcccaggaagctctgcctctgggcacagtgacctcagtggggtgaggggagctctccccatagctgggctgcggcccaaccccaccccctcaggctatgccagggggtgttgccaggggcacccgggcatcgccagtctagcccactccttcataaagccctcgcatcccaggagcgagcagagccagagcaggttggagaggagacgcatcacctccgctgctcgc

SEQ ID NO: 58 - DNA, 24 bp, Artificial Sequence
  Exemplary Taqman probe for quantification of constructs
tctggctcaccgtcctcttcattt

SEQ ID NO: 59 - DNA, 20 bp, Artificial Sequence
  Exemplary forward qPCR primer for quantification of constructs
caaacactccaccagcattg

SEQ ID NO: 60 - DNA, 20 bp, Artificial Sequence
  Exemplary reverse qPCR primer for quantification of constructs
cagccacaacgaggatcata

SEQ ID NO: 61 - DNA, 1010 bp, Artificial Sequence
  Exemplary Human GJB2 promoter
aagcttccgcagaatcctatcagtttccccctttcgtgctgtgtgcatcgagcaggaaggggcttggcaggttttacctgccctctttcctttctgaaaagtctgggcctcctcaccccgaaaggagtcacctccttgcagttccccagttgcgaaaagaggaggaagttggctgggccgggggccgcggggggcaccctccgcagatggcgggacccccctgccggccatggcaaaaacgaggcttgtctctcccaccgcccccaaccttagtccttggcacattgttgaaagtaattgaataaaatcggaaattcgagaaggcgttcgttcggattggtgagattttgaggggagaaagaagcggggacttcgccggcaccagcggcgccccctcctcggccaccgttaacccccattccagagggcactgccccgccacccagcctaggtccccctgcgagagcctcgcgggcccgcgcagcctccgcgactcgaacagatcttcagtccttggaggaatgcctgtttctctaacaataaaaaattaaagaagcgctcataaatgccaagtcctctcgcactatgcggagtacagaggacaacgaccacagccatccctgaaccccgcccacggcacagcgccggagccggggtctggggcgccgcttcctggggggtcccgactctcagccgcccccgcttcacccgggccgccaaggggctgggggaggcggcgctcggggtaaccgggggagactcagggcgctgggggcacttggggaactcatgggggctcaaaggaactaggagatcgggacctcgaaggggacttggggggttcggggctttcgggggcggtcgggggttcgcggacccgggaagctctgaggacccagaggccgggcgcgctccgcccgcggcgccgccccctccgtaactttcccagtctccgagggaagaggcggggtgtggggtgcggttaaaaggcgccacggcgggagacaggt

SEQ ID NO: 62 - DNA, 680 bp, Artificial Sequence
  Exemplary Human GFAP enhancer-promoter
gaacatatcctggtgtggagtaggggacgctgctctgacagaggctcgggggcctgagctggctctgtgagctggggaggaggcagacagccaggccttgtctgcaagcagacctggcagcattgggctggccgccccccagggcctcctcttcatgcccagtgaatgactcaccttggcacagacacaatgttcggggtgggcacagtgcctgcttcccgccgcaccccagcccccctcaaatgccttccgagaagcccattgagcagggggcttgcattgcaccccagcctgacagcctggcatcttgggataaaagcagcacagccccctaggggctgcccttgctgtgtggcgccaccggcggtggagaacaaggctctattcagcctgtgcccaggaaaggggatcaggggatgcccaggcatggacagtgggtggcagggggggagaggagggctgtctgcttcccagaagtccaaggacacaaatgggtgaggggagctctccccatagctgggctgcggcccaaccccaccccctcaggctatgccagggggtgttgccaggggcacccgggcatcgccagtctagcccactccttcataaagccctcgcatcccaggagcgagcagagccagagcaggttggagaggagacgcatcacctccgctgctcgc

SEQ ID NO: 63 - DNA, 382 bp, Artificial Sequence
  Exemplary CMV enhancer
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggt

SEQ ID NO: 64 - DNA, 245 bp, Artificial Sequence
  Exemplary chimeric intron
ggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagctagagcctctgctaaccatgttcatgccttcttctttttcctacag

SEQ ID NO: 65 - DNA, 1044 bp, Artificial Sequence; identical to SEQ ID NO: 27
  Exemplary GJB2 enhancer

SEQ ID NO: 66 - DNA, 362 bp, Artificial Sequence
  Exemplary 5′ UTR Sequence
gttgcggccccgcagcgcccgcgcgctcctctccccgactcggagcccctcggcggcgcccggcccaggacccgcctaggagcgcaggagccccagcgcagagaccccaacgccgagacccccgccccggccccgccgcgcttcctcccgacgcagtttaggacccttgttcgcgaagaggtggtgtgcggctgagacccgcgtcctcaggacggttccatcagtgcctcgatcctgccccactggaggaggaaggcagcccgaacagcgctcacctaactaacagctgctgagagctgggttccgtggccatgcacctgggactgccttgagaagcgtgagcaaaccgcccagagtagaag

SEQ ID NO: 67 - DNA, 1407 bp, Artificial Sequence
  Exemplary 3′ UTR Sequence
gaaatagacagcatgagagggatgaggcaacccgtgctcagctgtcaaggctcagtcgctagcatttcccaacacaaagattctgaccttaaatgcaaccatttgaaacccctgtaggcctcaggtgaaactccagatgccacaatggagctctgctcccctaaagcctcaaaacaaaggcctaattctatgcctgtcttaattttctttcacttaagttagttccactgagaccccaggctgttaggggttattggtgtaaggtactttcatattttaaacagaggatatcggcatttgtttctttctctgaggacaagagaaaaaagccaggttccacagaggacacagagaaggtttgggtgtcctcctggggttctttttgccaactttccccacgttaaaggtgaacattggttctttcatttgctttggaagttttaatctctaacagtggacaaagttaccagtgccttaaactctgttacactttttggaagtgaaaactttgtagtatgataggttattttgatgtaaagatgttctggataccattatatgttccccctgtttcagaggctcagattgtaatatgtaaatggtatgtcattcgctactatgatttaatttgaaatatggtcttttggttatgaatactttgcagcacagctgagaggctgtctgttgtattcattgtggtcatagcacctaacaacattgtagcctcaatcgagtgagacagactagaagttcctagtgatggcttatgatagcaaatggcctcatgtcaaatatttagatgtaattttgtgtaagaaatacagactggatgtaccaccaactactacctgtaatgacaggcctgtccaacacatctcccttttccatgactgtggtagccagcatcggaaagaacgctgatttaaagaggtcgcttgggaattttattgacacagtaccatttaatggggaggacaaaatggggcaggggagggagaagtttctgtcgttaaaaacagatttggaaagactggactctaaagtctgttgattaaagatgagctttgtctacttcaaaagtttgtttgcttaccccttcagcctccaattttttaagtgaaaatatagctaataacatgtgaaaagaatagaagctaaggtttagataaatattgagcagatctataggaagattgaacctgaatattgccattatgcttgacatggtttccaaaaaatggtactccacatatttcagtgagggtaagtattttcctgttgtcaagaatagcattgtaaaagcattttgtaataataaagaatagctttaatgatatgcttgtaactaaaataattttgtaatgtatcaaatacatttaaaacattaaaatataatctctataataa

SEQ ID NO: 68 - DNA, 1248 bp, Artificial Sequence
  Exemplary 3′ UTR Sequence
cgcattgcccagttgttagattaagaaatagacagcatgagagggatgaggcaacccgtgctcagctgtcaaggctcagtcgctagcatttcccaacacaaagattctgaccttaaatgcaaccatttgaaacccctgtaggcctcaggtgaaactccagatgccacaatggagcctctgctcccctaaagcctcaaaacaaaggcctaattctatgcctgtcttaattttctttcacttaagttagttccactgagaccccaggctgttaggggttattggtgtaaggtactttcatattttaaacagaggatatcggcatttgtttctttctctgaggacaagagaaaaaagccaggttccacagaggacacagagaaggtttgggtgtcctcctggggttctttttgccaactttccccacgttaaaggtgaacattggttctttcatttgctttggaagttttaatctctaacagtggacaaagttaccagtgccttaaactctgttacactttttggaagtgaaaactttgtagtatgataggttattttgatgtaaagatgttctggataccattatatgttccccctgtttcagaggctcagattgtaatatgtaaatggtatgtcattcgctactatgatttaatttgaaatatggtcttttggttatgaatactttgcagcacagctgagaggctgtctgttgtattcattgtggtcatagcacctaacaacattgtagcctcaatcgagtgagacagactagaagttcctagtgatggcttatgatagcaaatggcctcatgtcaaatatttagatgtaattttgtgtaagaaatacagactggatgtaccaccaactactacctgtaatgacaggcctgtccaacacatctcccttttccatgactgtggtagccagcatcggaaagaacgctgatttaaagaggtcgcttgggaattttattgacacagtaccatttaatggggaggacaaaatggggcaggggagggagaagtttctgtcgttaaaaacagatttggaaagactggactctaaagtctgttgattaaagatgagctttgtctacttcaaaagtttgtttgcttaccccttcagcctccaattttttaagtgaaaatatagctaataacatgtgaaaagaatagaagctaaggtttagataaatattgagcagatctataggaagattgaacctgaatattgccattatgcttgac

SEQ ID NO: 69 - DNA, 1372 bp, Artificial Sequence
  Exemplary 3′ UTR Sequence
gagctcagtgtgagttctaccattgccaaactcgagcagtgaattctaccagtgccataggatccagtgtgagttctaccattgccaaaggtacccagtgaattctaccagtgccatagttaaccgcattgcccagttgttagattaagaaatagacagcatgagagggatgaggcaacccgtgctcagctgtcaaggctcagtcgctagcatttcccaacacaaagattctgaccttaaatgcaaccatttgaaacccctgtaggcctcaggtgaaactccagatgccacaatggagcctctgctcccctaaagcctcaaaacaaaggcctaattctatgcctgtcttaattttctttcacttaagttagttccactgagaccccaggctgttaggggttattggtgtaaggtactttcatattttaaacagaggatatcggcatttgtttctttctctgaggacaagagaaaaaagccaggttccacagaggacacagagaaggtttgggtgtcctcctggggttctttttgccaactttccccacgttaaaggtgaacattggttctttcatttgctttggaagttttaatctctaacagtggacaaagttaccagtgccttaaactctgttacactttttggaagtgaaaactttgtagtatgataggttattttgatgtaaagatgttctggataccattatatgttccccctgtttcagaggctcagattgtaatatgtaaatggtatgtcattcgctactatgatttaatttgaaatatggtcttttggttatgaatactttgcagcacagctgagaggctgtctgttgtattcattgtggtcatagcacctaacaacattgtagcctcaatcgagtgagacagactagaagttcctagtgatggcttatgatagcaaatggcctcatgtcaaatatttagatgtaattttgtgtaagaaatacagactggatgtaccaccaactactacctgtaatgacaggcctgtccaacacatctcccttttccatgactgtggtagccagcatcggaaagaacgctgatttaaagaggtcgcttgggaattttattgacacagtaccatttaatggggaggacaaaatggggcaggggagggagaagtttctgtcgttaaaaacagatttggaaagactggactctaaagtctgttgattaaagatgagctttgtctacttcaaaagtttgtttgcttaccccttcagcctccaattttttaagtgaaaatatagctaataacatgtgaaaagaatagaagctaaggtttagataaatattgagcagatctataggaagattgaacctgaatattgccattatgcttgac

SEQ ID NO: 70 - DNA, 14 bp, Artificial Sequence
  Exemplary cloning site J
gcggccgcacgcgt

SEQ ID NO: 71 - DNA, 17 bp, Artificial Sequence
  Exemplary cloning site K
gcggccgcacgcgtggt

SEQ ID NO: 72 - DNA, 33 bp, Artificial Sequence
  Exemplary cloning site L
ctcctgggcaacgtgctggttattgtgaccggt

SEQ ID NO: 73 - DNA, 11 bp, Artificial Sequence
  Exemplary cloning site M
cgctagccacc

SEQ ID NO: 74 - DNA, 17 bp, Artificial Sequence
  Exemplary cloning site N
accggtcgctagccacc

SEQ ID NO: 75 - DNA, 21 bp, Artificial Sequence
  Exemplary cloning site O
gagctcgctgatcagcctcga

SEQ ID NO: 76 - DNA, 34 bp, Artificial Sequence
  Exemplary cloning site P
aagcttgaattcagctgacgtgcctcggaccgct

SEQ ID NO: 77 - DNA, 12 bp, Artificial Sequence
  Exemplary linker sequence
ggatcccgggct

SEQ ID NO: 78 - DNA, 24 bp, Artificial Sequence
  Exemplary mRNA destabilizing domain sequence
agtgtgagttctaccattgccaaa

SEQ ID NO: 79 - DNA, 22 bp, Artificial Sequence
  Exemplary mRNA destabilizing domain sequence
agtgaattctaccagtgccata

SEQ ID NO: 80 - DNA, 124 bp, Artificial Sequence
  Exemplary mRNA destabilizing domain Sequence
gagctcagtgtgagttctaccattgccaaactcgagcagtgaattctaccagtgccataggatccagtgtgagttctaccattgccaaaggtacccagtgaattctaccagtgccatagttaac

SEQ ID NO: 81 - DNA, 69 bp, Artificial Sequence
  Exemplary 3xFLAG tag sequence with stop codon
gactacaaagaccatgacggtgattataaagatcatgacatcgactacaaggatgacgatgacaagtaa

SEQ ID NO: 82 - DNA, 4786 bp, Artificial Sequence
  Exemplary Construct sequence
ctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgaccggtgttgcggccccgcagcgcccgcgcgctcctctccccgactcggagcccctcggcggcgcccggcccaggacccgcctaggagcgcaggagccccagcgcagagaccccaacgccgagacccccgccccggccccgccgcgcttcctcccgacgcagtttaggacccttgttcgcgaagaggtggtgtgcggctgagacccgcgtcctcaggacggttccatcagtgcctcgatcctgccccactggaggaggaaggcagcccgaacagcgctcacctaactaacagctgctgagagctgggttccgtggccatgcacctgggactgccttgagaagcgtgagcaaaccgcccagagtagaagcgctagccaccatggattggggcacgctgcagacgatcctggggggtgtgaacaaacactccaccagcattggaaagatctggctcaccgtcctcttcatttttcgcattatgatcctcgttgtggctgcaaaggaggtgtggggagatgagcaggccgactttgtctgcaacaccctgcagccaggctgcaagaacgtgtgctacgatcactacttccccatctcccacatccggctatgggccctgcagctgatcttcgtgtccacgccagcgctcctagtggccatgcacgtggcctaccggagacatgagaagaagaggaagttcatcaagggggagataaagagtgaatttaaggacatcgaggagatcaaaacccagaaggtccgcatcgaaggctccctgtggtggacctacacaagcagcatcttcttccgggtcatcttcgaagccgccttcatgtacgtcttctatgtcatgtacgacggcttctccatgcagcggctggtgaagtgcaacgcctggccttgtcccaacactgtggactgctttgtgtcccggcccacggagaagactgtcttcacagtgttcatgattgcagtgtctggaatttgcatcctgctgaatgtcactgaattgtgttatttgctaattagatattgttctgggaagtcaaaaaagccagttggatcccgggctgactacaaagaccatgacggtgattataaagatcatgacatcgactacaaggatgacgatgacaagtaagaaatagacagcatgagagggatgaggcaacccgtgctcagctgtcaaggctcagtcgctagcatttcccaacacaaagattctgaccttaaatgcaaccatttgaaacccctgtaggcctcaggtgaaactccagatgccacaatggagctctgctcccctaaagcctcaaaacaaaggcctaattctatgcctgtcttaattttctttcacttaagttagttccactgagaccccaggctgttaggggttattggtgtaaggtactttcatattttaaacagaggatatcggcatttgtttctttctctgaggacaagagaaaaaagccaggttccacagaggacacagagaaggtttgggtgtcctcctggggttctttttgccaactttccccacgttaaaggtgaacattggttctttcatttgctttggaagttttaatctctaacagtggacaaagttaccagtgccttaaactctgttacactttttggaagtgaaaactttgtagtatgataggttattttgatgtaaagatgttctggataccattatatgttccccctgtttcagaggctcagattgtaatatgtaaatggtatgtcattcgctactatgatttaatttgaaatatggtcttttggttatgaatactttgcagcacagctgagaggctgtctgttgtattcattgtggtcatagcacctaacaacattgtagcctcaatcgagtgagacagactagaagttcctagtgatggcttatgatagcaaatggcctcatgtcaaatatttagatgtaattttgtgtaagaaatacagactggatgtaccaccaactactacctgtaatgacaggcctgtccaacacatctcccttttccatgactgtggtagccagcatcggaaagaacgctgatttaaagaggtcgcttgggaattttattgacacagtaccatttaatggggaggacaaaatggggcaggggagggagaagtttctgtcgttaaaaacagatttggaaagactggactctaaagtctgttgattaaagatgagctttgtctacttcaaaagtttgtttgcttaccccttcagcctccaattttttaagtgaaaatatagctaataacatgtgaaaagaatagaagctaaggtttagataaatattgagcagatctataggaagattgaacctgaatattgccattatgcttgacatggtttccaaaaaatggtactccacatatttcagtgagggtaagtattttcctgttgtcaagaatagcattgtaaaagcattttgtaataataaagaatagctttaatgatatgcttgtaactaaaataattttgtaatgtatcaaatacatttaaaacattaaaatataatctctataataagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggaagcttgaattcagctgacgtgcctcggaccgctaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 83 - DNA, 4018 bp, Artificial Sequence
  Exemplary Construct sequence
ctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagctagagcctctgctaaccatgttcatgccttcttctttttcctacagctcctgggcaacgtgctggttattgtgaccggtgttgcggccccgcagcgcccgcgcgctcctctccccgactcggagcccctcggcggcgcccggcccaggacccgcctaggagcgcaggagccccagcgcagagaccccaacgccgagacccccgccccggccccgccgcgcttcctcccgacgcagtttaggacccttgttcgcgaagaggtggtgtgcggctgagacccgcgtcctcaggacggttccatcagtgcctcgatcctgccccactggaggaggaaggcagcccgaacagcgctcacctaactaacagctgctgagagctgggttccgtggccatgcacctgggactgccttgagaagcgtgagcaaaccgcccagagtagaagcgctagccaccatggattggggcacgctgcagacgatcctggggggtgtgaacaaacactccaccagcattggaaagatctggctcaccgtcctcttcatttttcgcattatgatcctcgttgtggctgcaaaggaggtgtggggagatgagcaggccgactttgtctgcaacaccctgcagccaggctgcaagaacgtgtgctacgatcactacttccccatctcccacatccggctatgggccctgcagctgatcttcgtgtccacgccagcgctcctagtggccatgcacgtggcctaccggagacatgagaagaagaggaagttcatcaagggggagataaagagtgaatttaaggacatcgaggagatcaaaacccagaaggtccgcatcgaaggctccctgtggtggacctacacaagcagcatcttcttccgggtcatcttcgaagccgccttcatgtacgtcttctatgtcatgtacgacggcttctccatgcagcggctggtgaagtgcaacgcctggccttgtcccaacactgtggactgctttgtgtcccggcccacggagaagactgtcttcacagtgttcatgattgcagtgtctggaatttgcatcctgctgaatgtcactgaattgtgttatttgctaattagatattgttctgggaagtcaaaaaagccagttggatcccgggctgactacaaagaccatgacggtgattataaagatcatgacatcgactacaaggatgacgatgacaagtaagaaatagacagcatgagagggatgaggcaacccgtgctcagctgtcaaggctcagtcgctagcatttcccaacacaaagattctgaccttaaatgcaaccatttgaaacccctgtaggcctcaggtgaaactccagatgccacaatggagctctgctcccctaaagcctcaaaacaaaggcctaattctatgcctgtcttaattttctttcacttaagttagttccactgagaccccaggctgttaggggttattggtgtaaggtactttcatattttaaacagaggatatcggcatttgtttctttctctgaggacaagagaaaaaagccaggttccacagaggacacagagaaggtttgggtgtcctcctggggttctttttgccaactttccccacgttaaaggtgaacattggttctttcatttgctttggaagttttaatctctaacagtggacaaagttaccagtgccttaaactctgttacactttttggaagtgaaaactttgtagtatgataggttattttgatgtaaagatgttctggataccattatatgttccccctgtttcagaggctcagattgtaatatgtaaatggtatgtcattcgctactatgatttaatttgaaatatggtcttttggttatgaatactttgcagcacagctgagaggctgtctgttgtattcattgtggtcatagcacctaacaacattgtagcctcaatcgagtgagacagactagaagttcctagtgatggcttatgatagcaaatggcctcatgtcaaatatttagatgtaattttgtgtaagaaatacagactggatgtaccaccaactactacctgtaatgacaggcctgtccaacacatctcccttttccatgactgtggtagccagcatcggaaagaacgctgatttaaagaggtcgcttgggaattttattgacacagtaccatttaatggggaggacaaaatggggcaggggagggagaagtttctgtcgttaaaaacagatttggaaagactggactctaaagtctgttgattaaagatgagctttgtctacttcaaaagtttgtttgcttaccccttcagcctccaattttttaagtgaaaatatagctaataacatgtgaaaagaatagaagctaaggtttagataaatattgagcagatctataggaagattgaacctgaatattgccattatgcttgacatggtttccaaaaaatggtactccacatatttcagtgagggtaagtattttcctgttgtcaagaatagcattgtaaaagcattttgtaataataaagaatagctttaatgatatgcttgtaactaaaataattttgtaatgtatcaaatacatttaaaacattaaaatataatctctataataagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggaagcttgaattcagctgacgtgcctcggaccgctaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 84 - DNA, 4508 bp, Artificial Sequence
  Exemplary Construct sequence
ctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtaagcttccgcagaatcctatcagtttccccctttcgtgctgtgtgcatcgagcaggaaggggcttggcaggttttacctgccctctttcctttctgaaaagtctgggcctcctcaccccgaaaggagtcacctccttgcagttccccagttgcgaaaagaggaggaagttggctgggccgggggccgcggggggcaccctccgcagatggcgggacccccctgccggccatggcaaaaacgaggcttgtctctcccaccgcccccaaccttagtccttggcacattgttgaaagtaattgaataaaatcggaaattcgagaaggcgttcgttcggattggtgagattttgaggggagaaagaagcggggacttcgccggcaccagcggcgccccctcctcggccaccgttaacccccattccagagggcactgccccgccacccagcctaggtccccctgcgagagcctcgcgggcccgcgcagcctccgcgactcgaacagatcttcagtccttggaggaatgcctgtttctctaacaataaaaaattaaagaagcgctcataaatgccaagtcctctcgcactatgcggagtacagaggacaacgaccacagccatccctgaaccccgcccacggcacagcgccggagccggggtctggggcgccgcttcctggggggtcccgactctcagccgcccccgcttcacccgggccgccaaggggctgggggaggcggcgctcggggtaaccgggggagactcagggcgctgggggcacttggggaactcatgggggctcaaaggaactaggagatcgggacctcgaaggggacttggggggttcggggctttcgggggcggtcgggggttcgcggacccgggaagctctgaggacccagaggccgggcgcgctccgcccgcggcgccgccccctccgtaactttcccagtctccgagggaagaggcggggtgtggggtgcggttaaaaggcgccacggcgggagacaggtctcctgggcaacgtgctggttattgtgaccggtgttgcggccccgcagcgcccgcgcgctcctctccccgactcggagcccctcggcggcgcccggcccaggacccgcctaggagcgcaggagccccagcgcagagaccccaacgccgagacccccgccccggccccgccgcgcttcctcccgacgcagtttaggacccttgttcgcgaagaggtggtgtgcggctgagacccgcgtcctcaggacggttccatcagtgcctcgatcctgccccactggaggaggaaggcagcccgaacagcgctcacctaactaacagctgctgagagctgggttccgtggccatgcacctgggactgccttgagaagcgtgagcaaaccgcccagagtagaagcgctagccaccatggattggggcacgctgcagacgatcctggggggtgtgaacaaacactccaccagcattggaaagatctggctcaccgtcctcttcatttttcgcattatgatcctcgttgtggctgcaaaggaggtgtggggagatgagcaggccgactttgtctgcaacaccctgcagccaggctgcaagaacgtgtgctacgatcactacttccccatctcccacatccggctatgggccctgcagctgatcttcgtgtccacgccagcgctcctagtggccatgcacgtggcctaccggagacatgagaagaagaggaagttcatcaagggggagataaagagtgaatttaaggacatcgaggagatcaaaacccagaaggtccgcatcgaaggctccctgtggtggacctacacaagcagcatcttcttccgggtcatcttcgaagccgccttcatgtacgtcttctatgtcatgtacgacggcttctccatgcagcggctggtgaagtgcaacgcctggccttgtcccaacactgtggactgctttgtgtcccggcccacggagaagactgtcttcacagtgttcatgattgcagtgtctggaatttgcatcctgctgaatgtcactgaattgtgttatttgctaattagatattgttctgggaagtcaaaaaagccagttggatcccgggctgactacaaagaccatgacggtgattataaagatcatgacatcgactacaaggatgacgatgacaagtaagaaatagacagcatgagagggatgaggcaacccgtgctcagctgtcaaggctcagtcgctagcatttcccaacacaaagattctgaccttaaatgcaaccatttgaaacccctgtaggcctcaggtgaaactccagatgccacaatggagctctgctcccctaaagcctcaaaacaaaggcctaattctatgcctgtcttaattttctttcacttaagttagttccactgagaccccaggctgttaggggttattggtgtaaggtactttcatattttaaacagaggatatcggcatttgtttctttctctgaggacaagagaaaaaagccaggttccacagaggacacagagaaggtttgggtgtcctcctggggttctttttgccaactttccccacgttaaaggtgaacattggttctttcatttgctttggaagttttaatctctaacagtggacaaagttaccagtgccttaaactctgttacactttttggaagtgaaaactttgtagtatgataggttattttgatgtaaagatgttctggataccattatatgttccccctgtttcagaggctcagattgtaatatgtaaatggtatgtcattcgctactatgatttaatttgaaatatggtcttttggttatgaatactttgcagcacagctgagaggctgtctgttgtattcattgtggtcatagcacctaacaacattgtagcctcaatcgagtgagacagactagaagttcctagtgatggcttatgatagcaaatggcctcatgtcaaatatttagatgtaattttgtgtaagaaatacagactggatgtaccaccaactactacctgtaatgacaggcctgtccaacacatctcccttttccatgactgtggtagccagcatcggaaagaacgctgatttaaagaggtcgcttgggaattttattgacacagtaccatttaatggggaggacaaaatggggcaggggagggagaagtttctgtcgttaaaaacagatttggaaagactggactctaaagtctgttgattaaagatgagctttgtctacttcaaaagtttgtttgcttaccccttcagcctccaattttttaagtgaaaatatagctaataacatgtgaaaagaatagaagctaaggtttagataaatattgagcagatctataggaagattgaacctgaatattgccattatgcttgacatggtttccaaaaaatggtactccacatatttcagtgagggtaagtattttcctgttgtcaagaatagcattgtaaaagcattttgtaataataaagaatagctttaatgatatgcttgtaactaaaataattttgtaatgtatcaaatacatttaaaacattaaaatataatctctataataagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggaagcttgaattcagctgacgtgcctcggaccgctaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 85 - DNA, 4178 bp, Artificial Sequence
  Exemplary Construct sequence
ctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtgaacatatcctggtgtggagtaggggacgctgctctgacagaggctcgggggcctgagctggctctgtgagctggggaggaggcagacagccaggccttgtctgcaagcagacctggcagcattgggctggccgccccccagggcctcctcttcatgcccagtgaatgactcaccttggcacagacacaatgttcggggtgggcacagtgcctgcttcccgccgcaccccagcccccctcaaatgccttccgagaagcccattgagcagggggcttgcattgcaccccagcctgacagcctggcatcttgggataaaagcagcacagccccctaggggctgcccttgctgtgtggcgccaccggcggtggagaacaaggctctattcagcctgtgcccaggaaaggggatcaggggatgcccaggcatggacagtgggtggcagggggggagaggagggctgtctgcttcccagaagtccaaggacacaaatgggtgaggggagctctccccatagctgggctgcggcccaaccccaccccctcaggctatgccagggggtgttgccaggggcacccgggcatcgccagtctagcccactccttcataaagccctcgcatcccaggagcgagcagagccagagcaggttggagaggagacgcatcacctccgctgctcgcctcctgggcaacgtgctggttattgtgaccggtgttgcggccccgcagcgcccgcgcgctcctctccccgactcggagcccctcggcggcgcccggcccaggacccgcctaggagcgcaggagccccagcgcagagaccccaacgccgagacccccgccccggccccgccgcgcttcctcccgacgcagtttaggacccttgttcgcgaagaggtggtgtgcggctgagacccgcgtcctcaggacggttccatcagtgcctcgatcctgccccactggaggaggaaggcagcccgaacagcgctcacctaactaacagctgctgagagctgggttccgtggccatgcacctgggactgccttgagaagcgtgagcaaaccgcccagagtagaagcgctagccaccatggattggggcacgctgcagacgatcctggggggtgtgaacaaacactccaccagcattggaaagatctggctcaccgtcctcttcatttttcgcattatgatcctcgttgtggctgcaaaggaggtgtggggagatgagcaggccgactttgtctgcaacaccctgcagccaggctgcaagaacgtgtgctacgatcactacttccccatctcccacatccggctatgggccctgcagctgatcttcgtgtccacgccagcgctcctagtggccatgcacgtggcctaccggagacatgagaagaagaggaagttcatcaagggggagataaagagtgaatttaaggacatcgaggagatcaaaacccagaaggtccgcatcgaaggctccctgtggtggacctacacaagcagcatcttcttccgggtcatcttcgaagccgccttcatgtacgtcttctatgtcatgtacgacggcttctccatgcagcggctggtgaagtgcaacgcctggccttgtcccaacactgtggactgctttgtgtcccggcccacggagaagactgtcttcacagtgttcatgattgcagtgtctggaatttgcatcctgctgaatgtcactgaattgtgttatttgctaattagatattgttctgggaagtcaaaaaagccagttggatcccgggctgactacaaagaccatgacggtgattataaagatcatgacatcgactacaaggatgacgatgacaagtaagaaatagacagcatgagagggatgaggcaacccgtgctcagctgtcaaggctcagtcgctagcatttcccaacacaaagattctgaccttaaatgcaaccatttgaaacccctgtaggcctcaggtgaaactccagatgccacaatggagctctgctcccctaaagcctcaaaacaaaggcctaattctatgcctgtcttaattttctttcacttaagttagttccactgagaccccaggctgttaggggttattggtgtaaggtactttcatattttaaacagaggatatcggcatttgtttctttctctgaggacaagagaaaaaagccaggttccacagaggacacagagaaggtttgggtgtcctcctggggttctttttgccaactttccccacgttaaaggtgaacattggttctttcatttgctttggaagttttaatctctaacagtggacaaagttaccagtgccttaaactctgttacactttttggaagtgaaaactttgtagtatgataggttattttgatgtaaagatgttctggataccattatatgttccccctgtttcagaggctcagattgtaatatgtaaatggtatgtcattcgctactatgatttaatttgaaatatggtcttttggttatgaatactttgcagcacagctgagaggctgtctgttgtattcattgtggtcatagcacctaacaacattgtagcctcaatcgagtgagacagactagaagttcctagtgatggcttatgatagcaaatggcctcatgtcaaatatttagatgtaattttgtgtaagaaatacagactggatgtaccaccaactactacctgtaatgacaggcctgtccaacacatctcccttttccatgactgtggtagccagcatcggaaagaacgctgatttaaagaggtcgcttgggaattttattgacacagtaccatttaatggggaggacaaaatggggcaggggagggagaagtttctgtcgttaaaaacagatttggaaagactggactctaaagtctgttgattaaagatgagctttgtctacttcaaaagtttgtttgcttaccccttcagcctccaattttttaagtgaaaatatagctaataacatgtgaaaagaatagaagctaaggtttagataaatattgagcagatctataggaagattgaacctgaatattgccattatgcttgacatggtttccaaaaaatggtactccacatatttcagtgagggtaagtattttcctgttgtcaagaatagcattgtaaaagcattttgtaataataaagaatagctttaatgatatgcttgtaactaaaataattttgtaatgtatcaaatacatttaaaacattaaaatataatctctataataagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggaagcttgaattcagctgacgtgcctcggaccgctaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 86 - DNA, 3798 bp, Artificial Sequence
  Exemplary Construct sequence
ctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtggtgaacatatcctggtgtggagtaggggacgctgctctgacagaggctcgggggcctgagctggctctgtgagctggggaggaggcagacagccaggccttgtctgcaagcagacctggcagcattgggctggccgccccccagggcctcctcttcatgcccagtgaatgactcaccttggcacagacacaatgttcggggtgggcacagtgcctgcttcccgccgcaccccagcccccctcaaatgccttccgagaagcccattgagcagggggcttgcattgcaccccagcctgacagcctggcatcttgggataaaagcagcacagccccctaggggctgcccttgctgtgtggcgccaccggcggtggagaacaaggctctattcagcctgtgcccaggaaaggggatcaggggatgcccaggcatggacagtgggtggcagggggggagaggagggctgtctgcttcccagaagtccaaggacacaaatgggtgaggggagctctccccatagctgggctgcggcccaaccccaccccctcaggctatgccagggggtgttgccaggggcacccgggcatcgccagtctagcccactccttcataaagccctcgcatcccaggagcgagcagagccagagcaggttggagaggagacgcatcacctccgctgctcgcctcctgggcaacgtgctggttattgtgaccggtgttgcggccccgcagcgcccgcgcgctcctctccccgactcggagcccctcggcggcgcccggcccaggacccgcctaggagcgcaggagccccagcgcagagaccccaacgccgagacccccgccccggccccgccgcgcttcctcccgacgcagtttaggacccttgttcgcgaagaggtggtgtgcggctgagacccgcgtcctcaggacggttccatcagtgcctcgatcctgccccactggaggaggaaggcagcccgaacagcgctcacctaactaacagctgctgagagctgggttccgtggccatgcacctgggactgccttgagaagcgtgagcaaaccgcccagagtagaagcgctagccaccatggattggggcacgctgcagacgatcctggggggtgtgaacaaacactccaccagcattggaaagatctggctcaccgtcctcttcatttttcgcattatgatcctcgttgtggctgcaaaggaggtgtggggagatgagcaggccgactttgtctgcaacaccctgcagccaggctgcaagaacgtgtgctacgatcactacttccccatctcccacatccggctatgggccctgcagctgatcttcgtgtccacgccagcgctcctagtggccatgcacgtggcctaccggagacatgagaagaagaggaagttcatcaagggggagataaagagtgaatttaaggacatcgaggagatcaaaacccagaaggtccgcatcgaaggctccctgtggtggacctacacaagcagcatcttcttccgggtcatcttcgaagccgccttcatgtacgtcttctatgtcatgtacgacggcttctccatgcagcggctggtgaagtgcaacgcctggccttgtcccaacactgtggactgctttgtgtcccggcccacggagaagactgtcttcacagtgttcatgattgcagtgtctggaatttgcatcctgctgaatgtcactgaattgtgttatttgctaattagatattgttctgggaagtcaaaaaagccagttggatcccgggctgactacaaagaccatgacggtgattataaagatcatgacatcgactacaaggatgacgatgacaagtaagaaatagacagcatgagagggatgaggcaacccgtgctcagctgtcaaggctcagtcgctagcatttcccaacacaaagattctgaccttaaatgcaaccatttgaaacccctgtaggcctcaggtgaaactccagatgccacaatggagctctgctcccctaaagcctcaaaacaaaggcctaattctatgcctgtcttaattttctttcacttaagttagttccactgagaccccaggctgttaggggttattggtgtaaggtactttcatattttaaacagaggatatcggcatttgtttctttctctgaggacaagagaaaaaagccaggttccacagaggacacagagaaggtttgggtgtcctcctggggttctttttgccaactttccccacgttaaaggtgaacattggttctttcatttgctttggaagttttaatctctaacagtggacaaagttaccagtgccttaaactctgttacactttttggaagtgaaaactttgtagtatgataggttattttgatgtaaagatgttctggataccattatatgttccccctgtttcagaggctcagattgtaatatgtaaatggtatgtcattcgctactatgatttaatttgaaatatggtcttttggttatgaatactttgcagcacagctgagaggctgtctgttgtattcattgtggtcatagcacctaacaacattgtagcctcaatcgagtgagacagactagaagttcctagtgatggcttatgatagcaaatggcctcatgtcaaatatttagatgtaattttgtgtaagaaatacagactggatgtaccaccaactactacctgtaatgacaggcctgtccaacacatctcccttttccatgactgtggtagccagcatcggaaagaacgctgatttaaagaggtcgcttgggaattttattgacacagtaccatttaatggggaggacaaaatggggcaggggagggagaagtttctgtcgttaaaaacagatttggaaagactggactctaaagtctgttgattaaagatgagctttgtctacttcaaaagtttgtttgcttaccccttcagcctccaattttttaagtgaaaatatagctaataacatgtgaaaagaatagaagctaaggtttagataaatattgagcagatctataggaagattgaacctgaatattgccattatgcttgacatggtttccaaaaaatggtactccacatatttcagtgagggtaagtattttcctgttgtcaagaatagcattgtaaaagcattttgtaataataaagaatagctttaatgatatgcttgtaactaaaataattttgtaatgtatcaaatacatttaaaacattaaaatataatctctataataagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggaagcttgaattcagctgacgtgcctcggaccgctaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 87 - DNA, 4745 bp, Artificial Sequence
  Exemplary Construct sequence
ctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgaccggtgttgcggccccgcagcgcccgcgcgctcctctccccgactcggagcccctcggcggcgcccggcccaggacccgcctaggagcgcaggagccccagcgcagagaccccaacgccgagacccccgccccggccccgccgcgcttcctcccgacgcagtttaggacccttgttcgcgaagaggtggtgtgcggctgagacccgcgtcctcaggacggttccatcagtgcctcgatcctgccccactggaggaggaaggcagcccgaacagcgctcacctaactaacagctgctgagagctgggttccgtggccatgcacctgggactgccttgagaagcgtgagcaaaccgcccagagtagaagcgctagccaccatggattggggcacgctgcagacgatcctggggggtgtgaacaaacactccaccagcattggaaagatctggctcaccgtcctcttcatttttcgcattatgatcctcgttgtggctgcaaaggaggtgtggggagatgagcaggccgactttgtctgcaacaccctgcagccaggctgcaagaacgtgtgctacgatcactacttccccatctcccacatccggctatgggccctgcagctgatcttcgtgtccacgccagcgctcctagtggccatgcacgtggcctaccggagacatgagaagaagaggaagttcatcaagggggagataaagagtgaatttaaggacatcgaggagatcaaaacccagaaggtccgcatcgaaggctccctgtggtggacctacacaagcagcatcttcttccgggtcatcttcgaagccgccttcatgtacgtcttctatgtcatgtacgacggcttctccatgcagcggctggtgaagtgcaacgcctggccttgtcccaacactgtggactgctttgtgtcccggcccacggagaagactgtcttcacagtgttcatgattgcagtgtctggaatttgcatcctgctgaatgtcactgaattgtgttatttgctaattagatattgttctgggaagtcaaaaaagccagttggatcccgggctgactacaaagaccatgacggtgattataaagatcatgacatcgactacaaggatgacgatgacaagtaagagctcagtgtgagttctaccattgccaaactcgagcagtgaattctaccagtgccataggatccagtgtgagttctaccattgccaaaggtacccagtgaattctaccagtgccatagttaaccgcattgcccagttgttagattaagaaatagacagcatgagagggatgaggcaacccgtgctcagctgtcaaggctcagtcgctagcatttcccaacacaaagattctgaccttaaatgcaaccatttgaaacccctgtaggcctcaggtgaaactccagatgccacaatggagcctctgctcccctaaagcctcaaaacaaaggcctaattctatgcctgtcttaattttctttcacttaagttagttccactgagaccccaggctgttaggggttattggtgtaaggtactttcatattttaaacagaggatatcggcatttgtttctttctctgaggacaagagaaaaaagccaggttccacagaggacacagagaaggtttgggtgtcctcctggggttctttttgccaactttccccacgttaaaggtgaacattggttctttcatttgctttggaagttttaatctctaacagtggacaaagttaccagtgccttaaactctgttacactttttggaagtgaaaactttgtagtatgataggttattttgatgtaaagatgttctggataccattatatgttccccctgtttcagaggctcagattgtaatatgtaaatggtatgtcattcgctactatgatttaatttgaaatatggtcttttggttatgaatactttgcagcacagctgagaggctgtctgttgtattcattgtggtcatagcacctaacaacattgtagcctcaatcgagtgagacagactagaagttcctagtgatggcttatgatagcaaatggcctcatgtcaaatatttagatgtaattttgtgtaagaaatacagactggatgtaccaccaactactacctgtaatgacaggcctgtccaacacatctcccttttccatgactgtggtagccagcatcggaaagaacgctgatttaaagaggtcgcttgggaattttattgacacagtaccatttaatggggaggacaaaatggggcaggggagggagaagtttctgtcgttaaaaacagatttggaaagactggactctaaagtctgttgattaaagatgagctttgtctacttcaaaagtttgtttgcttaccccttcagcctccaattttttaagtgaaaatatagctaataacatgtgaaaagaatagaagctaaggtttagataaatattgagcagatctataggaagattgaacctgaatattgccattatgcttgacgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggaagcttgaattcagctgacgtgcctcggaccgctaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 88 - DNA, 4712 bp, Artificial Sequence
  Exemplary Construct sequence
ctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtcttcttctggagtcttttctggaataattctgggagtgggctcagcctgcgggagagtaacatttttataacttgatagatgtagctgagatgcctcccagaggggagacccgcctctcctccggcagctgtgcacgtaggcttgttcccagcagcctggccagggtggtccacctggtgtttctcatcttctttccccggagcgctgactcctgcgcgtcctcttggaagactcttgacaggacgggtgttttatgggtgtgattcagtgtcctcttgcatcagttcaatgtggtggtgttcaatcaacccttgtagcgttagcaaaatttgctcaagtcattccgcaggaatgtctgtgtcttgcttccaagaaagcttgtaagtgccggcaacaggccaagcagctcacaaacctgaccacaagcctgtgagtaattgtggggcagcacttagcagtcttttattttcgacttattaaagtctcatcttggcctcaccttctccctggaaggtggcgtgggtgggaaccactgggtcagatctttttcacccttgccgtggagccagtttcctgttgcatgtgggggaagcaacatgtggtgaagagtatagaaaacgaaaacatgtgggtacagtatgtataagtggagggaacaaactcataattccaactagtttctcatgagagactcatgaatcattgtggtagttctcaatataaacttaatctaggccggatgtggtggctcacacctgtaatctcagcactctgggtggatcacttgaggtcaggagtttgagaccagtctgaccaacatggagaaaccccatcgctactaaaaatacaaaattatccagatgtggtggctcacacctgtaatcccagcactttgggaggctgaggcgggtggatcacttgaggtcaggagtttgagaccagcctgaccaacatggagaaactgtgtctctactaaaaatacaaaattagctgggcgtggtgacgcatgcctgtaatcccagctatttggaggccgaagcaggaagcttccgcagaatcctatcagtttccccctttcgtgctgtgtgcatcgagcaggaaggggcttggcaggttttacctgccctctttcctttctgaaaagtctgggcctcctcaccccgaaaggagtcacctccttgcagttccccagttgcgaaaagaggaggaagttggctgggccgggggccgcggggggcaccctccgcagatggcgggacccccctgccggccatggcaaaaacgaggcttgtctctcccaccgcccccaaccttagtccttggcacattgttgaaagtaattgaataaaatcggaaattcgagaaggcgttcgttcggattggtgagattttgaggggagaaagaagcggggacttcgccggcaccagcggcgccccctcctcggccaccgttaacccccattccagagggcactgccccgccacccagcctaggtccccctgcgagagcctcgcgggcccgcgcagcctccgcgactcgaacagatcttcagtccttggaggaatgcctgtttctctaacaataaaaaattaaagaagcgctcataaatgccaagtcctctcgcactatgcggagtacagaggacaacgaccacagccatccctgaaccccgcccacggcacagcgccggagccggggtctggggcgccgcttcctggggggtcccgactctcagccgcccccgcttcacccgggccgccaaggggctgggggaggcggcgctcggggtaaccgggggagactcagggcgctgggggcacttggggaactcatgggggctcaaaggaactaggagatcgggacctcgaaggggacttggggggttcggggctttcgggggcggtcgggggttcgcggacccgggaagctctgaggacccagaggccgggcgcgctccgcccgcggcgccgccccctccgtaactttcccagtctccgagggaagaggcggggtgtggggtgcggttaaaaggcgccacggcgggagacaggtgttgcggccccgcagcgcccgcgcgctcctctccccgactcggagcccctcggcggcgcccggcccaggacccgcctaggagcgcaggagccccagcgcagagaccccaacgccgagacccccgccccggccccgccgcgcttcctcccgacgcagagcaaaccgcccagagtagaagaccggtcgctagccaccatggattggggcacgctgcagacgatcctggggggtgtgaacaaacactccaccagcattggaaagatctggctcaccgtcctcttcatttttcgcattatgatcctcgttgtggctgcaaaggaggtgtggggagatgagcaggccgactttgtctgcaacaccctgcagccaggctgcaagaacgtgtgctacgatcactacttccccatctcccacatccggctatgggccctgcagctgatcttcgtgtccacgccagcgctcctagtggccatgcacgtggcctaccggagacatgagaagaagaggaagttcatcaagggggagataaagagtgaatttaaggacatcgaggagatcaaaacccagaaggtccgcatcgaaggctccctgtggtggacctacacaagcagcatcttcttccgggtcatcttcgaagccgccttcatgtacgtcttctatgtcatgtacgacggcttctccatgcagcggctggtgaagtgcaacgcctggccttgtcccaacactgtggactgctttgtgtcccggcccacggagaagactgtcttcacagtgttcatgattgcagtgtctggaatttgcatcctgctgaatgtcactgaattgtgttatttgctaattagatattgttctgggaagtcaaaaaagccagttggatcccgggctgactacaaagaccatgacggtgattataaagatcatgacatcgactacaaggatgacgatgacaagtaagaaatagacagcatgagagggatgaggcaacccgtgctcagctgtcaaggctcagtcgctagcatttcccaacacaaagattctgaccttaaatgcaaccatttgaaacccctgtaggcctcaggtgaaactccagatgccacaatggagctctgctcccctaaagcctcaaaacaaaggcctaattctatgcctgtcttaattttctttcacttaagttagttccactgagaccccaggctgttaggggttattggtgtaaggtactttcatattttaaacagaggatatcggcatttgtttctttctctgaggacaagagaaaaaagccaggttccacagaggacacagagaaggtttgggtgtcctcctggggttctttttgccaactttccccacgttaaaggtgaacattggttctttcatttgctttggaagttttaatctctaacagtggacaaagttaccagtgccttaaactctgttacactttttggaagtgaaaactttgtagtatgataggttattttgatgtaaagatgttctggataccattatatgttccccctgtttcagaggctcagattgtaatatgtaaatggtatgtcattcgctactatgatttaatttgaaatatggtcttttggttatgaatactttgcagcacagctgagaggctgtctgttgtattcattgtggtcatagcacctaacaacattgtagcctcaatcgagtgagacagactagaagttcctagtgatggcttatgatagcaaatggcctcatgtcaaatatttagatgtaattttgtgtaagaaatacagactggatgtaccaccaactactacctgtaatgacaggcctgtccaacacatctcccttttccatgactgtggtagccagcatcggaaagaacgctgatttaaagaggtcgcttgggaattttattgacacagtaccatttaatggggaggacaaaatggggcaggggagggagaagtttctgtcgttaaaaacagatttggaaagactggactctaaagtctgttgattaaagatgagctttgtctacttcaaaagtttgtttgcttaccccttcagcctccaattttttaagtgaaaatatagctaataacatgtgaaaagaatagaagctaaggtttagataaatattgagcagatctataggaagattgaacctgaatattgccattatgcttgacatggtttccaaaaaatggtactccacatatttcagtgagggtaagtattttcctgttgtcaagaatagcattgtaaaagcattttgtaataataaagaatagctttaatgatatgcttgtaactaaaataattttgtaatgtatcaaatacatttaaaacattaaaatataatctctataataaaagcttgaattcagctgacgtgcctcggaccgctaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 89 - DNA, 17 bp, Artificial Sequence
  Exemplary sNRP poly(A) signal sequence
aaataaaatacgaaatg

SEQ ID NO: 90 - DNA, 1188 bp, Artificial Sequence
  Exemplary GDF6 promoter
ccacaggtaactccgtcggcgtccacaggggggcaggagataccatactgcacagttgtacgtcttccatctgtttggtgtagaaaaatctaaccactacaagaatgccacgggcactgtggcagacagaagcagcgctacgccgcatcgcctttcagcgtgcaggcccaggaatgagcgaggcagtgggcggggaagacaggcacggggaatctggggacagataaaggaaactcgtgatggggcgaggctgggctgaagagaaacagattggggtagagctgcaaagggaggggtccactggaaggcgaggggggaggccgggaagagagagggtgggaaggcagtgtgagatgggagggcagtgtgagaagaaaagcaggctggggaagagggattggaatgcagaaggaacttggggaaggaggaagtcctgcaggcgggagggaaagaagagagggggagcagctaaagtctgcgtcagaagaggttggggactgcgagaggagaggctggggcctgcaggggagcgcagcagcttttagcatcgatccaaactctaaagactcgtggcctttgcctgacctcgagggtcgggaatagacgcctgtctttgtggagagcgatacccaaccgagaaaatggggctgttccgagctgggccctgcgcctggcccagggcgaggcttctctggctccgggctggcccctgaggggcagcacgcagcctgcagcagaggcgcctgctccaagctgtctcttgggggcgccgccgccgcttccctcctccggggccgctcgctcccaggaaagtggaggcggctggcgaggaccgagagccggggccgcgctgcggagggaccacacctccgggagttcgagggggaccctggcgcggcgggccagcctttcgggccggcagcgcccgccttcccccggtcagcgcttgcggcccgcgccgcgcgcaccgcccggcaaccccgcgcgcgtcccgcgggggcgctgcgtcttcctgccacaccggcgcaccgcggcccctctcccccacacctccggcccgcaccacccggctctcctcccaccctccccacccctcctctgccctccctccccattcctcccctcccggcgaggggcgggagggggcgtggcggggccggggtttgtgtggctgggacccggctcctc

SEQ ID NO: 91 - DNA, 128 bp, Artificial Sequence
  Exemplary hGJB2 basal promoter
aagctctgaggacccagaggccgggcgcgctccgcccgcggcgccgccccctccgtaactttcccagtctccgagggaagaggcggggtgtggggtgcggttaaaaggcgccacggcgggagacaggt

SEQ ID NO: 92 - DNA, 9 bp, Artificial Sequence
  Exemplary cloning site Q
ctcaccggt

SEQ ID NO: 93 - DNA, 8 bp, Artificial Sequence
  Synthetic barcode A
gtgtcacc

SEQ ID NO: 94 - DNA, 6787 bp, Artificial Sequence
  Exemplary Construct Sequence
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtggtccacaggtaactccgtcggcgtccacaggggggcaggagataccatactgcacagttgtacgtcttccatctgtttggtgtagaaaaatctaaccactacaagaatgccacgggcactgtggcagacagaagcagcgctacgccgcatcgcctttcagcgtgcaggcccaggaatgagcgaggcagtgggcggggaagacaggcacggggaatctggggacagataaaggaaactcgtgatggggcgaggctgggctgaagagaaacagattggggtagagctgcaaagggaggggtccactggaaggcgaggggggaggccgggaagagagagggtgggaaggcagtgtgagatgggagggcagtgtgagaagaaaagcaggctggggaagagggattggaatgcagaaggaacttggggaaggaggaagtcctgcaggcgggagggaaagaagagagggggagcagctaaagtctgcgtcagaagaggttggggactgcgagaggagaggctggggcctgcaggggagcgcagcagcttttagcatcgatccaaactctaaagactcgtggcctttgcctgacctcgagggtcgggaatagacgcctgtctttgtggagagcgatacccaaccgagaaaatggggctgttccgagctgggccctgcgcctggcccagggcgaggcttctctggctccgggctggcccctgaggggcagcacgcagcctgcagcagaggcgcctgctccaagctgtctcttgggggcgccgccgccgcttccctcctccggggccgctcgctcccaggaaagtggaggcggctggcgaggaccgagagccggggccgcgctgcggagggaccacacctccgggagttcgagggggaccctggcgcggcgggccagcctttcgggccggcagcgcccgccttcccccggtcagcgcttgcggcccgcgccgcgcgcaccgcccggcaaccccgcgcgcgtcccgcgggggcgctgcgtcttcctgccacaccggcgcaccgcggcccctctcccccacacctccggcccgcaccacccggctctcctcccaccctccccacccctcctctgccctccctccccattcctcccctcccggcgaggggcgggagggggcgtggcggggccggggtttgtgtggctgggacccggctcctcaagctctgaggacccagaggccgggcgcgctccgcccgcggcgccgccccctccgtaactttcccagtctccgagggaagaggcggggtgtggggtgcggttaaaaggcgccacggcgggagacaggtctcaccggtgtgtcaccgttgcggccccgcagcgcccgcgcgctcctctccccgactcggagcccctcggcggcgcccggcccaggacccgcctaggagcgcaggagccccagcgcagagaccccaacgccgagacccccgccccggccccgccgcgcttcctcccgacgcagtttaggacccttgttcgcgaagaggtggtgtgcggctgagacccgcgtcctcaggacggttccatcagtgcctcgatcctgccccactggaggaggaaggcagcccgaacagcgctcacctaactaacagctgctgagagctgggttccgtggccatgcacctgggactgccttgagaagcgtgagcaaaccgcccagagtagaagcgctagccaccatggattggggcacgctgcagacgatcctggggggtgtgaacaaacactccaccagcattggaaagatctggctcaccgtcctcttcatttttcgcattatgatcctcgttgtggctgcaaaggaggtgtggggagatgagcaggccgactttgtctgcaacaccctgcagccaggctgcaagaacgtgtgctacgatcactacttccccatctcccacatccggctatgggccctgcagctgatcttcgtgtccacgccagcgctcctagtggccatgcacgtggcctaccggagacatgagaagaagaggaagttcatcaagggggagataaagagtgaatttaaggacatcgaggagatcaaaacccagaaggtccgcatcgaaggctccctgtggtggacctacacaagcagcatcttcttccgggtcatcttcgaagccgccttcatgtacgtcttctatgtcatgtacgacggcttctccatgcagcggctggtgaagtgcaacgcctggccttgtcccaacactgtggactgctttgtgtcccggcccacggagaagactgtcttcacagtgttcatgattgcagtgtctggaatttgcatcctgctgaatgtcactgaattgtgttatttgctaattagatattgttctgggaagtcaaaaaagccagttggatcccgggctgactacaaagaccatgacggtgattataaagatcatgacatcgactacaaggatgacgatgacaagtaagaaatagacagcatgagagggatgaggcaacccgtgctcagctgtcaaggctcagtcgctagcatttcccaacacaaagattctgaccttaaatgcaaccatttgaaacccctgtaggcctcaggtgaaactccagatgccacaatggagctctgctcccctaaagcctcaaaacaaaggcctaattctatgcctgtcttaattttctttcacttaagttagttccactgagaccccaggctgttaggggttattggtgtaaggtactttcatattttaaacagaggatatcggcatttgtttctttctctgaggacaagagaaaaaagccaggttccacagaggacacagagaaggtttgggtgtcctcctggggttctttttgccaactttccccacgttaaaggtgaacattggttctttcatttgctttggaagttttaatctctaacagtggacaaagttaccagtgccttaaactctgttacactttttggaagtgaaaactttgtagtatgataggttattttgatgtaaagatgttctggataccattatatgttccccctgtttcagaggctcagattgtaatatgtaaatggtatgtcattcgctactatgatttaatttgaaatatggtcttttggttatgaatactttgcagcacagctgagaggctgtctgttgtattcattgtggtcatagcacctaacaacattgtagcctcaatcgagtgagacagactagaagttcctagtgatggcttatgatagcaaatggcctcatgtcaaatatttagatgtaattttgtgtaagaaatacagactggatgtaccaccaactactacctgtaatgacaggcctgtccaacacatctcccttttccatgactgtggtagccagcatcggaaagaacgctgatttaaagaggtcgcttgggaattttattgacacagtaccatttaatggggaggacaaaatggggcaggggagggagaagtttctgtcgttaaaaacagatttggaaagactggactctaaagtctgttgattaaagatgagctttgtctacttcaaaagtttgtttgcttaccccttcagcctccaattttttaagtgaaaatatagctaataacatgtgaaaagaatagaagctaaggtttagataaatattgagcagatctataggaagattgaacctgaatattgccattatgcttgacatggtttccaaaaaatggtactccacatatttcagtgagggtaagtattttcctgttgtcaagaatagcattgtaaaagcattttgtaataataaagaatagctttaatgatatgcttgtaactaaaataattttgtaatgtatcaaatacatttaaaacattaaaatataatctctataataagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggaagcttgaattcagctgacgtgcctcggaccgctaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgaacaataaaactgtctgcttacataaacagtaatacaaggggtgttatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 95 - DNA, 1513 bp, Artificial Sequence
  Exemplary IGFBP2 promoter
aagaaacttgcccgagtttacacagctagtaaatggttgcattagtcaggacagctagcctatattacaataacaaccctctcaaatcctaatggcttaaaacaacagaggtttaatttatactcattagctgttcaaggcaggaggctctattctctaatccatacagtcactcaggatccaggctggtggagaccctgccatattgtagcctcaccatttaaaacatgaagaagatagaaagtgaggagtcatgtaggttttgttccgttgcctcaggctaggagtgacaggtcacttcatctcactcacagctcactgcccacaactagtcacttgtgactgtgcgagttaagcttctgtgtgtgaaggaaggaaaagagaatgggataaaggtgaacatcagcaggctctaccacagtagtttgaaccaagacttgagcctaggtcatgtggcttcagaatctttgctcttaatcacactaaacagcctctgtaagtcatctttccttcatccagtgcctaagaacatgcagtccaatgccctcatccttcagaagaacttgagtgaactcagagaaattgagtagagtgccacagcatgcccaaggccacacaccctgaggttggcagtaggtcctgagttagagttgtcatttcttggctcccctggtagtagtggaaaggtaaggttttgacatactagttggatgaccacgggcaggtcacttaaattgtctaagcatcgtttgacccttgtaagaattaaatgaaatagcacctgtaaaagtgtctgcacggacttactgctgttagttttgttcctttcttcctgttgtcactgcacttccctgcctgttacccaggccatgcagaccagccaggccttcgacttacagtgcggataagattccaaatctccacggctggtttccatgctttcttccaggcttctgaggaccctgtgctctggtttcttctatttcttttctattacttttctgttactcttgagcacacttgctggaagcaatatgcatccagttctccctctcttgcctcattacactttgcagaacaactccaatcccttccaaccaagtagtccctttgaatttcttgtcacccaaggaatctctctgacaggggtctttgttagggtcacaccccaggagatggttgattatggctgagtccagcctggaatgatgggggttgggggcagcttgggtagatgactcagtaaatcaaacagaacaatgaaaggaggtcatgcttgtccatctgcattattgaagacagccataaatggccttaccccagagcgggtctgtcacacctggagagctgatctgacctctccaagacccctgcaactgagtgttctgggatctgtcctgcaacaagtgcctcgagatttgtaggtgggggcccagagggagggggtctgcagacgaagggggcaggttttgcggggcacttagggttctcataggttgtagtcacgagctcc

SEQ ID NO: 96 - DNA, 8 bp, Artificial Sequence
  Exemplary synthetic barcode B
cacaacct

SEQ ID NO: 97 - DNA, 7112 bp, Artificial Sequence
  Exemplary Construct Sequence
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtggtaagaaacttgcccgagtttacacagctagtaaatggttgcattagtcaggacagctagcctatattacaataacaaccctctcaaatcctaatggcttaaaacaacagaggtttaatttatactcattagctgttcaaggcaggaggctctattctctaatccatacagtcactcaggatccaggctggtggagaccctgccatattgtagcctcaccatttaaaacatgaagaagatagaaagtgaggagtcatgtaggttttgttccgttgcctcaggctaggagtgacaggtcacttcatctcactcacagctcactgcccacaactagtcacttgtgactgtgcgagttaagcttctgtgtgtgaaggaaggaaaagagaatgggataaaggtgaacatcagcaggctctaccacagtagtttgaaccaagacttgagcctaggtcatgtggcttcagaatctttgctcttaatcacactaaacagcctctgtaagtcatctttccttcatccagtgcctaagaacatgcagtccaatgccctcatccttcagaagaacttgagtgaactcagagaaattgagtagagtgccacagcatgcccaaggccacacaccctgaggttggcagtaggtcctgagttagagttgtcatttcttggctcccctggtagtagtggaaaggtaaggttttgacatactagttggatgaccacgggcaggtcacttaaattgtctaagcatcgtttgacccttgtaagaattaaatgaaatagcacctgtaaaagtgtctgcacggacttactgctgttagttttgttcctttcttcctgttgtcactgcacttccctgcctgttacccaggccatgcagaccagccaggccttcgacttacagtgcggataagattccaaatctccacggctggtttccatgctttcttccaggcttctgaggaccctgtgctctggtttcttctatttcttttctattacttttctgttactcttgagcacacttgctggaagcaatatgcatccagttctccctctcttgcctcattacactttgcagaacaactccaatcccttccaaccaagtagtccctttgaatttcttgtcacccaaggaatctctctgacaggggtctttgttagggtcacaccccaggagatggttgattatggctgagtccagcctggaatgatgggggttgggggcagcttgggtagatgactcagtaaatcaaacagaacaatgaaaggaggtcatgcttgtccatctgcattattgaagacagccataaatggccttaccccagagcgggtctgtcacacctggagagctgatctgacctctccaagacccctgcaactgagtgttctgggatctgtcctgcaacaagtgcctcgagatttgtaggtgggggcccagagggagggggtctgcagacgaagggggcaggttttgcggggcacttagggttctcataggttgtagtcacgagctccaagctctgaggacccagaggccgggcgcgctccgcccgcggcgccgccccctccgtaactttcccagtctccgagggaagaggcggggtgtggggtgcggttaaaaggcgccacggcgggagacaggtctcaccggtcacaacctgttgcggccccgcagcgcccgcgcgctcctctccccgactcggagcccctcggcggcgcccggcccaggacccgcctaggagcgcaggagccccagcgcagagaccccaacgccgagacccccgccccggccccgccgcgcttcctcccgacgcagtttaggacccttgttcgcgaagaggtggtgtgcggctgagacccgcgtcctcaggacggttccatcagtgcctcgatcctgccccactggaggaggaaggcagcccgaacagcgctcacctaactaacagctgctgagagctgggttccgtggccatgcacctgggactgccttgagaagcgtgagcaaaccgcccagagtagaagcgctagccaccatggattggggcacgctgcagacgatcctggggggtgtgaacaaacactccaccagcattggaaagatctggctcaccgtcctcttcatttttcgcattatgatcctcgttgtggctgcaaaggaggtgtggggagatgagcaggccgactttgtctgcaacaccctgcagccaggctgcaagaacgtgtgctacgatcactacttccccatctcccacatccggctatgggccctgcagctgatcttcgtgtccacgccagcgctcctagtggccatgcacgtggcctaccggagacatgagaagaagaggaagttcatcaagggggagataaagagtgaatttaaggacatcgaggagatcaaaacccagaaggtccgcatcgaaggctccctgtggtggacctacacaagcagcatcttcttccgggtcatcttcgaagccgccttcatgtacgtcttctatgtcatgtacgacggcttctccatgcagcggctggtgaagtgcaacgcctggccttgtcccaacactgtggactgctttgtgtcccggcccacggagaagactgtcttcacagtgttcatgattgcagtgtctggaatttgcatcctgctgaatgtcactgaattgtgttatttgctaattagatattgttctgggaagtcaaaaaagccagttggatcccgggctgactacaaagaccatgacggtgattataaagatcatgacatcgactacaaggatgacgatgacaagtaagaaatagacagcatgagagggatgaggcaacccgtgctcagctgtcaaggctcagtcgctagcatttcccaacacaaagattctgaccttaaatgcaaccatttgaaacccctgtaggcctcaggtgaaactccagatgccacaatggagctctgctcccctaaagcctcaaaacaaaggcctaattctatgcctgtcttaattttctttcacttaagttagttccactgagaccccaggctgttaggggttattggtgtaaggtactttcatattttaaacagaggatatcggcatttgtttctttctctgaggacaagagaaaaaagccaggttccacagaggacacagagaaggtttgggtgtcctcctggggttctttttgccaactttccccacgttaaaggtgaacattggttctttcatttgctttggaagttttaatctctaacagtggacaaagttaccagtgccttaaactctgttacactttttggaagtgaaaactttgtagtatgataggttattttgatgtaaagatgttctggataccattatatgttccccctgtttcagaggctcagattgtaatatgtaaatggtatgtcattcgctactatgatttaatttgaaatatggtcttttggttatgaatactttgcagcacagctgagaggctgtctgttgtattcattgtggtcatagcacctaacaacattgtagcctcaatcgagtgagacagactagaagttcctagtgatggcttatgatagcaaatggcctcatgtcaaatatttagatgtaattttgtgtaagaaatacagactggatgtaccaccaactactacctgtaatgacaggcctgtccaacacatctcccttttccatgactgtggtagccagcatcggaaagaacgctgatttaaagaggtcgcttgggaattttattgacacagtaccatttaatggggaggacaaaatggggcaggggagggagaagtttctgtcgttaaaaacagatttggaaagactggactctaaagtctgttgattaaagatgagctttgtctacttcaaaagtttgtttgcttaccccttcagcctccaattttttaagtgaaaatatagctaataacatgtgaaaagaatagaagctaaggtttagataaatattgagcagatctataggaagattgaacctgaatattgccattatgcttgacatggtttccaaaaaatggtactccacatatttcagtgagggtaagtattttcctgttgtcaagaatagcattgtaaaagcattttgtaataataaagaatagctttaatgatatgcttgtaactaaaataattttgtaatgtatcaaatacatttaaaacattaaaatataatctctataataagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggaagcttgaattcagctgacgtgcctcggaccgctaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgaacaataaaactgtctgcttacataaacagtaatacaaggggtgttatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 98 - DNA, 1097 bp, Artificial Sequence
  RBP7 promoter
cccatggctctgttaaaatcaaagaaacatcttttccaacagccctttcaaactcctcatcgcatctcactggctgattcagtcatttaaacctgcttctccctaaagctgatcactggctaagctaatagggtttccgggattggtttagcctgatactaatccaggtctaccttcaggagccagaccaaactgcctattggcattgcattcttgcagtagggaggggaggtatggatggtgtggagtccaccacaaggtccatgccagtctttgctgaaccagcatcagactccatcaagcaacagatgagaggttccatgataaagtggccctcagcaatccccatccattgctgtctaggaagaacagtgcttgtacacaggtttaggacctcagtcttggctgtaatcttctggtttactttgccagcaccaaacagaaggaaagaaagggctcaaatttgaccaaataaattatgcttctccttccagagataaccttgagtcctgtctaggaagatattagaattgtaaagaaaaaaaaaattactccttatcctatggcaagtggagtctatgtctacttcagctgaaattaaatcctgtccataatagatgacccttgctcaagctggccagaagccataccaaccagcacgaaggttaaaactattattagttttttctgtgattttcattttcaggccaagttttagaacaataagattttaagaataggaagtaagtaagatttctgcatatcctgttctcttagtcagctgaatttttttttttttttttttagtcctaactcagcctcccaaagtgctgggattacaggcgtgagccaccgcaccaagcctggaatctatgtcttacagttatgagaatcaacagctagctcattatgggcaaggtgatgtcactctggcttctcaatgaaaatggcatttctcccttggaaaaggtcatagccagtcagtcagtcagtcacgggagcgcagcggcttctaggggtgagtgggacccacgcggccccacctgctcctcccgcgcgcggccccacccccctgccccgccccgcctggtttatag

SEQ ID NO: 99 - DNA, 8 bp, Artificial Sequence
  Synthetic barcode C
cgtgtgtt

SEQ ID NO: 100 - DNA, 6696 bp, Artificial Sequence
  Exemplary Construct Sequence
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtggtcccatggctctgttaaaatcaaagaaacatcttttccaacagccctttcaaactcctcatcgcatctcactggctgattcagtcatttaaacctgcttctccctaaagctgatcactggctaagctaatagggtttccgggattggtttagcctgatactaatccaggtctaccttcaggagccagaccaaactgcctattggcattgcattcttgcagtagggaggggaggtatggatggtgtggagtccaccacaaggtccatgccagtctttgctgaaccagcatcagactccatcaagcaacagatgagaggttccatgataaagtggccctcagcaatccccatccattgctgtctaggaagaacagtgcttgtacacaggtttaggacctcagtcttggctgtaatcttctggtttactttgccagcaccaaacagaaggaaagaaagggctcaaatttgaccaaataaattatgcttctccttccagagataaccttgagtcctgtctaggaagatattagaattgtaaagaaaaaaaaaattactccttatcctatggcaagtggagtctatgtctacttcagctgaaattaaatcctgtccataatagatgacccttgctcaagctggccagaagccataccaaccagcacgaaggttaaaactattattagttttttctgtgattttcattttcaggccaagttttagaacaataagattttaagaataggaagtaagtaagatttctgcatatcctgttctcttagtcagctgaatttttttttttttttttttagtcctaactcagcctcccaaagtgctgggattacaggcgtgagccaccgcaccaagcctggaatctatgtcttacagttatgagaatcaacagctagctcattatgggcaaggtgatgtcactctggcttctcaatgaaaatggcatttctcccttggaaaaggtcatagccagtcagtcagtcagtcacgggagcgcagcggcttctaggggtgagtgggacccacgcggccccacctgctcctcccgcgcgcggccccacccccctgccccgccccgcctggtttatagaagctctgaggacccagaggccgggcgcgctccgcccgcggcgccgccccctccgtaactttcccagtctccgagggaagaggcggggtgtggggtgcggttaaaaggcgccacggcgggagacaggtctcaccggtcgtgtgttgttgcggccccgcagcgcccgcgcgctcctctccccgactcggagcccctcggcggcgcccggcccaggacccgcctaggagcgcaggagccccagcgcagagaccccaacgccgagacccccgccccggccccgccgcgcttcctcccgacgcagtttaggacccttgttcgcgaagaggtggtgtgcggctgagacccgcgtcctcaggacggttccatcagtgcctcgatcctgccccactggaggaggaaggcagcccgaacagcgctcacctaactaacagctgctgagagctgggttccgtggccatgcacctgggactgccttgagaagcgtgagcaaaccgcccagagtagaagcgctagccaccatggattggggcacgctgcagacgatcctggggggtgtgaacaaacactccaccagcattggaaagatctggctcaccgtcctcttcatttttcgcattatgatcctcgttgtggctgcaaaggaggtgtggggagatgagcaggccgactttgtctgcaacaccctgcagccaggctgcaagaacgtgtgctacgatcactacttccccatctcccacatccggctatgggccctgcagctgatcttcgtgtccacgccagcgctcctagtggccatgcacgtggcctaccggagacatgagaagaagaggaagttcatcaagggggagataaagagtgaatttaaggacatcgaggagatcaaaacccagaaggtccgcatcgaaggctccctgtggtggacctacacaagcagcatcttcttccgggtcatcttcgaagccgccttcatgtacgtcttctatgtcatgtacgacggcttctccatgcagcggctggtgaagtgcaacgcctggccttgtcccaacactgtggactgctttgtgtcccggcccacggagaagactgtcttcacagtgttcatgattgcagtgtctggaatttgcatcctgctgaatgtcactgaattgtgttatttgctaattagatattgttctgggaagtcaaaaaagccagttggatcccgggctgactacaaagaccatgacggtgattataaagatcatgacatcgactacaaggatgacgatgacaagtaagaaatagacagcatgagagggatgaggcaacccgtgctcagctgtcaaggctcagtcgctagcatttcccaacacaaagattctgaccttaaatgcaaccatttgaaacccctgtaggcctcaggtgaaactccagatgccacaatggagctctgctcccctaaagcctcaaaacaaaggcctaattctatgcctgtcttaattttctttcacttaagttagttccactgagaccccaggctgttaggggttattggtgtaaggtactttcatattttaaacagaggatatcggcatttgtttctttctctgaggacaagagaaaaaagccaggttccacagaggacacagagaaggtttgggtgtcctcctggggttctttttgccaactttccccacgttaaaggtgaacattggttctttcatttgctttggaagttttaatctctaacagtggacaaagttaccagtgccttaaactctgttacactttttggaagtgaaaactttgtagtatgataggttattttgatgtaaagatgttctggataccattatatgttccccctgtttcagaggctcagattgtaatatgtaaatggtatgtcattcgctactatgatttaatttgaaatatggtcttttggttatgaatactttgcagcacagctgagaggctgtctgttgtattcattgtggtcatagcacctaacaacattgtagcctcaatcgagtgagacagactagaagttcctagtgatggcttatgatagcaaatggcctcatgtcaaatatttagatgtaattttgtgtaagaaatacagactggatgtaccaccaactactacctgtaatgacaggcctgtccaacacatctcccttttccatgactgtggtagccagcatcggaaagaacgctgatttaaagaggtcgcttgggaattttattgacacagtaccatttaatggggaggacaaaatggggcaggggagggagaagtttctgtcgttaaaaacagatttggaaagactggactctaaagtctgttgattaaagatgagctttgtctacttcaaaagtttgtttgcttaccccttcagcctccaattttttaagtgaaaatatagctaataacatgtgaaaagaatagaagctaaggtttagataaatattgagcagatctataggaagattgaacctgaatattgccattatgcttgacatggtttccaaaaaatggtactccacatatttcagtgagggtaagtattttcctgttgtcaagaatagcattgtaaaagcattttgtaataataaagaatagctttaatgatatgcttgtaactaaaataattttgtaatgtatcaaatacatttaaaacattaaaatataatctctataataagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggaagcttgaattcagctgacgtgcctcggaccgctaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgaacaataaaactgtctgcttacataaacagtaatacaaggggtgttatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 101 - DNA, 735 bp, Artificial Sequence
  GJB6 promoter
aaatagcttccaacgtttccaccccaccagcccttgcaccactccctgtactggccctgagctttctagtcttgactgaaaagcggggaggcaatgtggtctctcctggtgcactgtcccgaggaaggcctgctccgcttccccggaggagtcttcaaaggatggaggtaattaataaaaacaacccctgtacctcctctaagtggtcattaattaataaagaacctccaggctcctataggagaggtctgtgcaccccgcgggctatgagaaggctggatcacccagaaagactgaggatgtgtcctggcaaaaacacagcctgcccctcacactgctccccacgggtgcactagggaggaagagttccctcgagggcctgagcaggcgccccacacctgcacccgtgcagagggggctgggcccgccctctgcgctcccgagggagagccctaccccctgcatccccggtaccccgttccctccaagggccggaaagagggccccgcgcactgtgcacttcttaggggtcccccaccctgcgcccccgccacgggaaaaaggtccccgctctgcgcatccggccccggagggacagccccggtcctgcactccttgctcctcagggggacggtccgcgcccagcggctagtgcgccccgggtaggtgggggcggggggctcgtcgagtgacagcgctcgcctcccgcagcccgcccgagccgcgtcagggcag

SEQ ID NO: 102 - DNA, 8 bp, Artificial Sequence
  Exemplary synthetic barcode D
tcgtgggt

SEQ ID NO: 103 - DNA, 6334 bp, Artificial Sequence
  Exemplary construct sequence
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtggtaaatagcttccaacgtttccaccccaccagcccttgcaccactccctgtactggccctgagctttctagtcttgactgaaaagcggggaggcaatgtggtctctcctggtgcactgtcccgaggaaggcctgctccgcttccccggaggagtcttcaaaggatggaggtaattaataaaaacaacccctgtacctcctctaagtggtcattaattaataaagaacctccaggctcctataggagaggtctgtgcaccccgcgggctatgagaaggctggatcacccagaaagactgaggatgtgtcctggcaaaaacacagcctgcccctcacactgctccccacgggtgcactagggaggaagagttccctcgagggcctgagcaggcgccccacacctgcacccgtgcagagggggctgggcccgccctctgcgctcccgagggagagccctaccccctgcatccccggtaccccgttccctccaagggccggaaagagggccccgcgcactgtgcacttcttaggggtcccccaccctgcgcccccgccacgggaaaaaggtccccgctctgcgcatccggccccggagggacagccccggtcctgcactccttgctcctcagggggacggtccgcgcccagcggctagtgcgccccgggtaggtgggggcggggggctcgtcgagtgacagcgctcgcctcccgcagcccgcccgagccgcgtcagggcagaagctctgaggacccagaggccgggcgcgctccgcccgcggcgccgccccctccgtaactttcccagtctccgagggaagaggcggggtgtggggtgcggttaaaaggcgccacggcgggagacaggtctcaccggttcgtgggtgttgcggccccgcagcgcccgcgcgctcctctccccgactcggagcccctcggcggcgcccggcccaggacccgcctaggagcgcaggagccccagcgcagagaccccaacgccgagacccccgccccggccccgccgcgcttcctcccgacgcagtttaggacccttgttcgcgaagaggtggtgtgcggctgagacccgcgtcctcaggacggttccatcagtgcctcgatcctgccccactggaggaggaaggcagcccgaacagcgctcacctaactaacagctgctgagagctgggttccgtggccatgcacctgggactgccttgagaagcgtgagcaaaccgcccagagtagaagcgctagccaccatggattggggcacgctgcagacgatcctggggggtgtgaacaaacactccaccagcattggaaagatctggctcaccgtcctcttcatttttcgcattatgatcctcgttgtggctgcaaaggaggtgtggggagatgagcaggccgactttgtctgcaacaccctgcagccaggctgcaagaacgtgtgctacgatcactacttccccatctcccacatccggctatgggccctgcagctgatcttcgtgtccacgccagcgctcctagtggccatgcacgtggcctaccggagacatgagaagaagaggaagttcatcaagggggagataaagagtgaatttaaggacatcgaggagatcaaaacccagaaggtccgcatcgaaggctccctgtggtggacctacacaagcagcatcttcttccgggtcatcttcgaagccgccttcatgtacgtcttctatgtcatgtacgacggcttctccatgcagcggctggtgaagtgcaacgcctggccttgtcccaacactgtggactgctttgtgtcccggcccacggagaagactgtcttcacagtgttcatgattgcagtgtctggaatttgcatcctgctgaatgtcactgaattgtgttatttgctaattagatattgttctgggaagtcaaaaaagccagttggatcccgggctgactacaaagaccatgacggtgattataaagatcatgacatcgactacaaggatgacgatgacaagtaagaaatagacagcatgagagggatgaggcaacccgtgctcagctgtcaaggctcagtcgctagcatttcccaacacaaagattctgaccttaaatgcaaccatttgaaacccctgtaggcctcaggtgaaactccagatgccacaatggagctctgctcccctaaagcctcaaaacaaaggcctaattctatgcctgtcttaattttctttcacttaagttagttccactgagaccccaggctgttaggggttattggtgtaaggtactttcatattttaaacagaggatatcggcatttgtttctttctctgaggacaagagaaaaaagccaggttccacagaggacacagagaaggtttgggtgtcctcctggggttctttttgccaactttccccacgttaaaggtgaacattggttctttcatttgctttggaagttttaatctctaacagtggacaaagttaccagtgccttaaactctgttacactttttggaagtgaaaactttgtagtatgataggttattttgatgtaaagatgttctggataccattatatgttccccctgtttcagaggctcagattgtaatatgtaaatggtatgtcattcgctactatgatttaatttgaaatatggtcttttggttatgaatactttgcagcacagctgagaggctgtctgttgtattcattgtggtcatagcacctaacaacattgtagcctcaatcgagtgagacagactagaagttcctagtgatggcttatgatagcaaatggcctcatgtcaaatatttagatgtaattttgtgtaagaaatacagactggatgtaccaccaactactacctgtaatgacaggcctgtccaacacatctcccttttccatgactgtggtagccagcatcggaaagaacgctgatttaaagaggtcgcttgggaattttattgacacagtaccatttaatggggaggacaaaatggggcaggggagggagaagtttctgtcgttaaaaacagatttggaaagactggactctaaagtctgttgattaaagatgagctttgtctacttcaaaagtttgtttgcttaccccttcagcctccaattttttaagtgaaaatatagctaataacatgtgaaaagaatagaagctaaggtttagataaatattgagcagatctataggaagattgaacctgaatattgccattatgcttgacatggtttccaaaaaatggtactccacatatttcagtgagggtaagtattttcctgttgtcaagaatagcattgtaaaagcattttgtaataataaagaatagctttaatgatatgcttgtaactaaaataattttgtaatgtatcaaatacatttaaaacattaaaatataatctctataataagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggaagcttgaattcagctgacgtgcctcggaccgctaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgaacaataaaactgtctgcttacataaacagtaatacaaggggtgttatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 104 - DNA, 1316 bp, Artificial Sequence
  PARM1 promoter
tgtacaggagatagtcagggaattagtaattttcaaagaggtgactttgaattcaaacttaaatatcatcttcagctgaaacaaagaaggggtgcagttatgaggaagtgaccaggtaaagcatggcaaacaaaggtaaagtttgttatgcgtatttaagtcagagccctctccattgataagagtttccagtaatttagtgccatccttttcttgctatagagttctcgtctctatctgagcacgcaaaaataacatgctttcttgctttcttgaagttgggcatggccattgacttgccttagcccatatttttctgtgaagtggtcttcaaaaacctatatttctgccatagagtcacttacttaacctgccctatttaaaggggctaatgcctgatagaatgtcgctgcataactccatctgtgtgtggtccctgcatccatgacaaccaaaacccagatgcagaaattgttcctaatcacatagattaccctagaaaccggaagggccttgaagtcaaaagcattcagagaacatgctgaacaaattgaatttgcagtttatctggccagggaggatggagaggggatgggcacttggtctgagtattttttgtttctcattccaacagaaattactagatttaccaaaaaatctacaagtggtagtgtgatagagtcaggcagaggaattgaccatagataaggtgctcaggactcctagagtcagcttctggtatgtgagaaagaagtgagaacagagcccatggcatatgaagaagatattacagaaaaaagaaagctgccttccacgcaaatcatttctttacaaaggcttgttaactcctgcagtgccaagaagctgaatgcagcggcagacatcctggttcgggccccaggaagctcagccgggtttaatgtggatgagggtttaatgatgtacacgcagaagtgttttgacaaatgaagaaggtcctcattcttggaacatgtgccggttctccgagggaactcctaaaaggctgtaagctcatgtaggaaaagctgagctagattcctaagggcagagatgtgctcacatttctttgcatccctagttcccagcacagtgcaaggcgctgcaaacatttgctgaacccagggtctcgtgtcttgactgtccagcagaggccgctctgggccggggctctcgggacctgagggctgagagaaggaaggccagggggtggcccagtcatcgccgcggggcccgggtgggaggggtttggcagcggcaggcgcggcggcggcggcggaggcggaggcggccccggg

SEQ ID NO: 105 - DNA, 8 bp, Artificial Sequence
  Exemplary synthetic barcode E
gcaaactg

SEQ ID NO: 106 - DNA, 6915 bp, Artificial Sequence
  Exemplary construct sequence
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtggttgtacaggagatagtcagggaattagtaattttcaaagaggtgactttgaattcaaacttaaatatcatcttcagctgaaacaaagaaggggtgcagttatgaggaagtgaccaggtaaagcatggcaaacaaaggtaaagtttgttatgcgtatttaagtcagagccctctccattgataagagtttccagtaatttagtgccatccttttcttgctatagagttctcgtctctatctgagcacgcaaaaataacatgctttcttgctttcttgaagttgggcatggccattgacttgccttagcccatatttttctgtgaagtggtcttcaaaaacctatatttctgccatagagtcacttacttaacctgccctatttaaaggggctaatgcctgatagaatgtcgctgcataactccatctgtgtgtggtccctgcatccatgacaaccaaaacccagatgcagaaattgttcctaatcacatagattaccctagaaaccggaagggccttgaagtcaaaagcattcagagaacatgctgaacaaattgaatttgcagtttatctggccagggaggatggagaggggatgggcacttggtctgagtattttttgtttctcattccaacagaaattactagatttaccaaaaaatctacaagtggtagtgtgatagagtcaggcagaggaattgaccatagataaggtgctcaggactcctagagtcagcttctggtatgtgagaaagaagtgagaacagagcccatggcatatgaagaagatattacagaaaaaagaaagctgccttccacgcaaatcatttctttacaaaggcttgttaactcctgcagtgccaagaagctgaatgcagcggcagacatcctggttcgggccccaggaagctcagccgggtttaatgtggatgagggtttaatgatgtacacgcagaagtgttttgacaaatgaagaaggtcctcattcttggaacatgtgccggttctccgagggaactcctaaaaggctgtaagctcatgtaggaaaagctgagctagattcctaagggcagagatgtgctcacatttctttgcatccctagttcccagcacagtgcaaggcgctgcaaacatttgctgaacccagggtctcgtgtcttgactgtccagcagaggccgctctgggccggggctctcgggacctgagggctgagagaaggaaggccagggggtggcccagtcatcgccgcggggcccgggtgggaggggtttggcagcggcaggcgcggcggcggcggcggaggcggaggcggccccgggaagctctgaggacccagaggccgggcgcgctccgcccgcggcgccgccccctccgtaactttcccagtctccgagggaagaggcggggtgtggggtgcggttaaaaggcgccacggcgggagacaggtctcaccggtgcaaactggttgcggccccgcagcgcccgcgcgctcctctccccgactcggagcccctcggcggcgcccggcccaggacccgcctaggagcgcaggagccccagcgcagagaccccaacgccgagacccccgccccggccccgccgcgcttcctcccgacgcagtttaggacccttgttcgcgaagaggtggtgtgcggctgagacccgcgtcctcaggacggttccatcagtgcctcgatcctgccccactggaggaggaaggcagcccgaacagcgctcacctaactaacagctgctgagagctgggttccgtggccatgcacctgggactgccttgagaagcgtgagcaaaccgcccagagtagaagcgctagccaccatggattggggcacgctgcagacgatcctggggggtgtgaacaaacactccaccagcattggaaagatctggctcaccgtcctcttcatttttcgcattatgatcctcgttgtggctgcaaaggaggtgtggggagatgagcaggccgactttgtctgcaacaccctgcagccaggctgcaagaacgtgtgctacgatcactacttccccatctcccacatccggctatgggccctgcagctgatcttcgtgtccacgccagcgctcctagtggccatgcacgtggcctaccggagacatgagaagaagaggaagttcatcaagggggagataaagagtgaatttaaggacatcgaggagatcaaaacccagaaggtccgcatcgaaggctccctgtggtggacctacacaagcagcatcttcttccgggtcatcttcgaagccgccttcatgtacgtcttctatgtcatgtacgacggcttctccatgcagcggctggtgaagtgcaacgcctggccttgtcccaacactgtggactgctttgtgtcccggcccacggagaagactgtcttcacagtgttcatgattgcagtgtctggaatttgcatcctgctgaatgtcactgaattgtgttatttgctaattagatattgttctgggaagtcaaaaaagccagttggatcccgggctgactacaaagaccatgacggtgattataaagatcatgacatcgactacaaggatgacgatgacaagtaagaaatagacagcatgagagggatgaggcaacccgtgctcagctgtcaaggctcagtcgctagcatttcccaacacaaagattctgaccttaaatgcaaccatttgaaacccctgtaggcctcaggtgaaactccagatgccacaatggagctctgctcccctaaagcctcaaaacaaaggcctaattctatgcctgtcttaattttctttcacttaagttagttccactgagaccccaggctgttaggggttattggtgtaaggtactttcatattttaaacagaggatatcggcatttgtttctttctctgaggacaagagaaaaaagccaggttccacagaggacacagagaaggtttgggtgtcctcctggggttctttttgccaactttccccacgttaaaggtgaacattggttctttcatttgctttggaagttttaatctctaacagtggacaaagttaccagtgccttaaactctgttacactttttggaagtgaaaactttgtagtatgataggttattttgatgtaaagatgttctggataccattatatgttccccctgtttcagaggctcagattgtaatatgtaaatggtatgtcattcgctactatgatttaatttgaaatatggtcttttggttatgaatactttgcagcacagctgagaggctgtctgttgtattcattgtggtcatagcacctaacaacattgtagcctcaatcgagtgagacagactagaagttcctagtgatggcttatgatagcaaatggcctcatgtcaaatatttagatgtaattttgtgtaagaaatacagactggatgtaccaccaactactacctgtaatgacaggcctgtccaacacatctcccttttccatgactgtggtagccagcatcggaaagaacgctgatttaaagaggtcgcttgggaattttattgacacagtaccatttaatggggaggacaaaatggggcaggggagggagaagtttctgtcgttaaaaacagatttggaaagactggactctaaagtctgttgattaaagatgagctttgtctacttcaaaagtttgtttgcttaccccttcagcctccaattttttaagtgaaaatatagctaataacatgtgaaaagaatagaagctaaggtttagataaatattgagcagatctataggaagattgaacctgaatattgccattatgcttgacatggtttccaaaaaatggtactccacatatttcagtgagggtaagtattttcctgttgtcaagaatagcattgtaaaagcattttgtaataataaagaatagctttaatgatatgcttgtaactaaaataattttgtaatgtatcaaatacatttaaaacattaaaatataatctctataataagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggaagcttgaattcagctgacgtgcctcggaccgctaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgaacaataaaactgtctgcttacataaacagtaatacaaggggtgttatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 107 - DNA, 22 bp, Artificial Sequence
  Exemplary mRNA destabilizing domain sequence
tccacatggagttgctgttaca

SEQ ID NO: 108 - DNA, 21 bp, Artificial Sequence
  Exemplary mRNA destabilizing domain sequence
ccgtggttctaccctgtggta

SEQ ID NO: 109 - DNA, 23 bp, Artificial Sequence
  Exemplary mRNA destabilizing domain sequence
ctatctgcactagatgcacctta

SEQ ID NO: 110 - DNA, 22 bp, Artificial Sequence
  Exemplary mRNA destabilizing domain sequence
cacaagatcggatctacgggtt

SEQ ID NO: 111 - DNA, 20 bp, Artificial Sequence
  Exemplary mRNA destabilizing domain sequence
ctgagtgtaggatgtttaca

SEQ ID NO: 112 - DNA, 22 bp, Artificial Sequence
  Exemplary mRNA destabilizing domain sequence
cacaaaccattatgtgctgcta